(19) United States
(12) Patent Application Publication
(10) Pub. No.: US 2020/0371091 A1
(43) Pub. Date: Nov. 26, 2020

(54) BCMA-TARGETING CHIMERIC ANTIGEN RECEPTOR, AND USES THEREOF

(71) Applicants: Novartis AG, Basel (CH); The Trustees of the University of Pennsylvania, Philadelphia, PA (US)

(72) Inventors: Iulian Pruteanu-Malinici, Medford, MA (US); Keith Mansfield, Shrewsbury, MA (US); Boris Engels, Arlington, MA (US); Jan J. Melenhorst, Cherry Hill, NJ (US); Adam David Cohen, Bala Cynwyd, PA (US); Edward A. Stadtmauer, Bryn Mawr, PA (US); Alfred Garfall, Wallingford, PA (US); Michael C. Milone, Cherry Hill, NJ (US); Joseph A. Fraietta, Williamstown, NJ (US)

(21) Appl. No.: 16/768,260

(22) PCT Filed: Nov. 30, 2018
(86) PCT No.: PCT/US2018/063255

§ 371 Date: May 29, 2020

Related U.S. Application Data
(60) Provisional application No. 62/752,010, filed on Oct. 29, 2018.
(60) Provisional application No. 62/593,043, filed on Nov. 30, 2017.

(51) Int. Cl. G01N 33/50; A61K 35/17; A61P 35/02; A61K 45/06; A61K 39/395; C07K 16/28; C07K 16/24

(52) U.S. Cl. CPC G01N 33/5088; A61K 2039/505; A61K 35/17; A61P 35/02; A61K 45/06; A61K 39/39541; C07K 16/2887; C07K 16/2809; C07K 16/283; C07K 16/2896; C07K 16/244; C07K 16/245; C07K 16/2818; C07K 16/2878; G01N 2800/52; C07K 2317/31; C07K 2317/76; G01N 33/505

(57) ABSTRACT

The invention provides compositions and methods for treating diseases associated with expression of BCMA. The invention also relates to a method of administering a BCMA-targeting chimeric antigen receptor (CAR) therapy and an additional therapeutic agent.

### RELATED APPLICATIONS

This application claims priority to U.S. Ser. No. 62/593,043 filed Nov. 30, 2017, and U.S. Ser. No. 62/752,010 filed Oct. 29, 2018, the contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 28, 2018, is named N2067-7137WO_SL.txt and is 1,411,518 bytes in size.

### FIELD OF THE INVENTION

The present invention relates generally to the use of cells engineered to express a chimeric antigen receptor targeting B-cell maturation antigen protein (BCMA), optionally in combination with an additional therapeutic agent, to treat a disease associated with the expression of BCMA. The invention further describes prognostic biomarkers for BCMA-targeted therapies.

### BACKGROUND OF THE INVENTION

BCMA is a tumor necrosis family receptor (TNFR) member expressed on cells of the B-cell lineage. BCMA expression is the highest on terminally differentiated B cells that assume the long lived plasma cell fate, including plasma cells, plasmablasts and a subpopulation of activated B cells and memory B cells. BCMA is involved in mediating the survival of plasma cells for maintaining long-term humoral immunity. The expression of BCMA has been recently linked to a number of cancers, autoimmune disorders, and infectious diseases. Cancers with increased expression of BCMA include some hematological cancers, such as multiple myeloma (MM), Hodgkin's and non-Hodgkin's lymphoma, diffuse large B-cell lymphoma (DLBCL), various leukemias (e.g., chronic lymphocytic leukaemia (CLL)), and glioblastoma.

Given the ongoing need for improved strategies for targeting diseases such as cancer, new compositions and methods for improving therapeutic agents that target BCMA, e.g., anti-BCMA chimeric antigen receptor (CAR) therapies, are highly desirable.

### SUMMARY OF THE INVENTION

The disclosure features, at least in part, a method of treating a disease or disorder associated with expression of B-cell maturation antigen (BCMA, also known as TNFRSF17, BCM, or CD269). In certain embodiments, the disorder is a cancer, e.g., a hematological cancer. In some embodiments, the disclosure features a BCMA CAR-expressing cell therapy, e.g., as a monotherapy or in a combination therapy with an additional therapeutic agent. In some embodiments, the BCMA CAR-expressing cell therapy is a cell (e.g., a population of cells) that expresses a CAR molecule that binds BCMA. In some embodiments, the combination therapy maintains or has better clinical effectiveness as compared to either therapy alone. In one embodiment, the BCMA CAR-expressing cell therapy and the additional therapeutic agent are present in a single dose form, or as two or more dose forms. In one embodiment, provided herein is a composition comprising a BCMA CAR-expressing cell therapy and an additional therapeutic agent for use as a medicament. In one embodiment, provided herein is a composition comprising a BCMA CAR-expressing cell therapy and an additional therapeutic agent for use in the treatment of a disease associated with expression of BCMA. In one aspect, provided herein is a kit comprising a BCMA CAR-expressing cell therapy and an additional therapeutic agent. In some embodiments, the disclosure additional features methods of evaluating or predicting a subject's responsiveness to a BCMA CAR-expressing cell therapy, or methods of evaluating or predicting the potency of a BCMA CAR-expressing cell therapy in a subject. In some embodiments, a BCMA-targeting CAR therapy is manufactured or administered based on the acquisition of a level of a biomarker from a patient sample.

In one aspect, this invention features methods of predicting in vivo expansion of BCMA CAR T cells in a subject. In another aspect, featured herein are methods of predicting a subject's responsiveness to BCMA CAR T cells. In some embodiments, a higher CD4+:CD8+ T cell ratio in a leukapheresis product isolated from the subject can be used to predict greater in vivo expansion of BCMA CAR T cells in the subject and/or greater clinical responses of the subject to the BCMA CAR T cells. In some embodiments, a lower CD4+:CD8+ T cell ratio in a leukapheresis product isolated from the subject can be used to predict weaker in vivo expansion of BCMA CAR T cells in the subject and/or weaker clinical responses of the subject to the BCMA CAR T cells. In some embodiments, a higher CD4+:CD8+ T cell ratio in a seed culture at the start of the manufacturing of the BCMA CAR T cells (e.g., in a leukapheresis product after monocytes are removed) can be used to predict greater in vivo expansion of BCMA CAR T cells in the subject and/or greater clinical responses of the subject to the BCMA CAR T cells. In some embodiments, a lower CD4+:CD8+ T cell ratio in a seed culture at the start of the manufacturing of the BCMA CAR T cells (e.g., in a leukapheresis product after monocytes are removed) can be used to predict weaker in vivo expansion of BCMA CAR T cells in the subject and/or weaker clinical responses of the subject to the BCMA CAR T cells. In some embodiments, a higher CD4+:CD8+ T cell ratio in the subject's peripheral blood and/or bone marrow prior to the administration of the BCMA CAR T cells can be used to predict greater in vivo expansion of BCMA CAR T cells in the subject. In some embodiments, a lower CD4+:CD8+ T cell ratio in the subject's peripheral blood and/or bone marrow prior to the administration of the BCMA CAR T cells can be used to predict weaker in vivo expansion of BCMA CAR T cells in the subject.

In some embodiments, a higher frequency of CD8+ T cells with an “early-memory” phenotype (e.g., a higher frequency of CD45RO−CD27+CD8+ T cells) in a leukapheresis product isolated from the subject can be used to predict greater in vivo expansion of BCMA CAR T cells in the subject and/or greater clinical responses of the subject to the BCMA CAR T cells. In some embodiments, a lower frequency of CD8+ T cells with an “early-memory” phenotype (e.g., a lower frequency of CD45RO-CD27+CD8+ T cells) in a leukapheresis product isolated from the subject can be used to predict weaker in vivo expansion of BCMA CAR T cells in the subject and/or weaker clinical responses of the subject to the BCMA CAR T cells.

In some embodiments, greater in vitro expansion of seeded cells from the subject during manufacturing of the BCMA CAR T cells can be used to predict greater in vivo expansion of BCMA CAR T cells in the subject. In some embodiments, weaker in vitro expansion of seeded cells from the subject during manufacturing of the BCMA CAR T cells can be used to predict weaker in vivo expansion of BCMA CAR T cells in the subject.

In one aspect, provided herein is a method of evaluating or predicting a subject's responsiveness to a BCMA CAR-expressing cell therapy, wherein the subject has a disease associated with the expression of BCMA, comprising:

acquiring a value for one, two, three, four, five, or all of:

(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,

(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(iv) the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)), or

(vi) the proliferation of seeded cells from the subject during manufacturing of the BCMA CAR-expressing cell therapy, e.g., as measured by population doublings by day 9 (PDL9), wherein:

(a) an increase in the value of one, two, three, four, five, or all of (i)-(vi), as compared to a reference value, e.g., a non-responder reference value, is indicative or predictive of increased responsiveness of the subject to the BCMA CAR-expressing cell therapy; or

(b) a decrease in the value of one, two, three, four, five, or all of (i)-(vi), as compared to a reference value, e.g., a responder reference value, is indicative or predictive of decreased responsiveness of the subject to the BCMA CAR-expressing cell therapy,

thereby evaluating or predicting the subject's responsiveness to the BCMA CAR-expressing cell therapy.

In some embodiments, the method comprises acquiring a value for the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy. In some embodiments, the method comprises acquiring a value for the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the proliferation of seeded cells from the subject during manufacturing of the BCMA CAR-expressing cell therapy, e.g., population doublings by day 9 (PDL9).

In some embodiments, the increase in the value of one, two, three, four, five, or all of (i)-(vi), as compared to a reference value, e.g., a non-responder reference value, is indicative or predictive of increased responsiveness of the subject to the BCMA CAR-expressing cell therapy. In some embodiments, the decrease in the value of one, two, three, four, five, or all of (i)-(vi), as compared to a reference value, e.g., a responder reference value, is indicative or predictive of decreased responsiveness of the subject to the BCMA CAR-expressing cell therapy.

In some embodiments, the increase in the value of one, two, three, four, five, or all of (i)-(vi), as compared to a reference value, e.g., a non-responder reference value, is indicative or predictive of one, two, three, or all of:

(a) increased responsiveness of the subject to the BCMA CAR-expressing cell therapy;

(b) the subject as a responder of the BCMA CAR-expressing cell therapy;

(c) the subject as suitable for the BCMA CAR-expressing cell therapy; or

(d) increased expansion of the BCMA CAR-expressing cell therapy in the subject, e.g., as measured by an assay disclosed herein, e.g., as measured by the copy number of CAR transgenes per μg DNA using qPCR.

In some embodiments, the increase in the value of one, two, three, four, five, or all of (i)-(vi), as compared to a reference value, e.g., a non-responder reference value, is indicative or predictive of increased responsiveness of the subject to the BCMA CAR-expressing cell therapy. In some embodiments, the increase in the value of one, two, three, four, five, or all of (i)-(vi), as compared to a reference value, e.g., a non-responder reference value, is indicative or predictive of the subject as a responder of the BCMA CAR-expressing cell therapy. In some embodiments, the increase in the value of one, two, three, four, five, or all of (i)-(vi), as compared to a reference value, e.g., a non-responder reference value, is indicative or predictive of the subject as suitable for the BCMA CAR-expressing cell therapy. In some embodiments, the increase in the value of one, two, three, four, five, or all of (i)-(vi), as compared to a reference value, e.g., a non-responder reference value, is indicative or predictive of increased expansion of the BCMA CAR-expressing cell therapy in the subject, e.g., as measured by an assay disclosed herein, e.g., as measured by the copy number of CAR transgenes per μg DNA using qPCR.

In some embodiments, the decrease in the value of one, two, three, four, five, or all of (i)-(vi), as compared to a reference value, e.g., a responder reference value, is indicative or predictive of one, two, or all of:

(a) decreased responsiveness of the subject to the BCMA CAR-expressing cell therapy;

(b) the subject as a non-responder of the BCMA CAR-expressing cell therapy; or

(c) decreased expansion of the BCMA CAR-expressing cell therapy in the subject, e.g., as measured by an assay disclosed herein, e.g., as measured by the copy number of CAR transgenes per μg DNA using qPCR.

In some embodiments, the decrease in the value of one, two, three, four, five, or all of (i)-(vi), as compared to a reference value, e.g., a responder reference value, is indicative or predictive of decreased responsiveness of the subject to the BCMA CAR-expressing cell therapy. In some embodiments, the decrease in the value of one, two, three, four, five, or all of (i)-(vi), as compared to a reference value, e.g., a responder reference value, is indicative or predictive of the subject as a non-responder of the BCMA CAR-expressing cell therapy. In some embodiments, the decrease in the value of one, two, three, four, five, or all of (i)-(vi), as compared to a reference value, e.g., a responder reference value, is indicative or predictive of decreased expansion of the BCMA CAR-expressing cell therapy in the subject, e.g., as measured by an assay disclosed herein, e.g., as measured by the copy number of CAR transgenes per μg DNA using qPCR.

In some embodiments, the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry. In some embodiments,

In some embodiments, the ratio being:

(1) greater than or equal to 1 (e.g., between 1 and 5, e.g., between 1 and 3.5); or

(2) greater than or equal to 1.6 (e.g., between 1.6 and 5, e.g., between 1.6 and 3.5), is indicative or predictive of one, two, three, or all of:

(a) increased responsiveness of the subject to the BCMA CAR-expressing cell therapy;

(b) the subject as a responder of the BCMA CAR-expressing cell therapy;

(c) the subject as suitable for the BCMA CAR-expressing cell therapy; or

(d) increased expansion of the BCMA CAR-expressing cell therapy in the subject, e.g., as measured by an assay disclosed herein, e.g., as measured by the copy number of CAR transgenes per μg DNA using qPCR.

In some embodiments, the ratio being less than 1 (e.g., between 0.001 and 1) is indicative or predictive of one, two, or all of:

(a) decreased responsiveness of the subject to the BCMA CAR-expressing cell therapy;

(b) the subject as a non-responder of the BCMA CAR-expressing cell therapy; or

(c) decreased expansion of the BCMA CAR-expressing cell therapy in the subject, e.g., as measured by an assay disclosed herein, e.g., as measured by the copy number of CAR transgenes per μg DNA using qPCR.

In some embodiments, the value for the level or activity of CD8+ Tscm (stem cell memory T cells) comprises the percentage of CD8+ Tscm (stem cell memory T cells) among CD8+ T cells, e.g., as measured by an assay disclosed herein, e.g., flow cytometry.

In some embodiments, the value for the level or activity of HLADR-CD95+CD27+CD8+ cells comprises the percentage of HLADR-CD95+CD27+CD8+ cells among CD8+ T cells, e.g., as measured by an assay disclosed herein, e.g., flow cytometry.

In some embodiments, the percentage of HLADR-CD95+CD27+CD8+ cells among CD8+ T cells being greater than or equal to 25% (e.g., between 30% and 90%, e.g., between 35% and 85%, e.g., between 40% and 80%, e.g., between 45% and 75%, e.g., between 50% and 75%) is indicative or predictive of one, two, three, or all of:

(a) increased responsiveness of the subject to the BCMA CAR-expressing cell therapy;

(b) the subject as a responder of the BCMA CAR-expressing cell therapy;

(c) the subject as suitable for the BCMA CAR-expressing cell therapy; or

(d) increased expansion of the BCMA CAR-expressing cell therapy in the subject, e.g., as measured by an assay disclosed herein, e.g., as measured by the copy number of CAR transgenes per μg DNA using qPCR.

In some embodiments, the percentage of HLADR-CD95+CD27+CD8+ cells among CD8+ T cells being less than 25% (e.g., between 0.1% and 25%, e.g., between 0.1% and 22%, e.g., between 0.1% and 20%, e.g., between 0.1% and 18%, e.g., between 0.1% and 15%)) is indicative or predictive of one, two, or all of:

(a) decreased responsiveness of the subject to the BCMA CAR-expressing cell therapy;

(b) the subject as a non-responder of the BCMA CAR-expressing cell therapy; or

(c) decreased expansion of the BCMA CAR-expressing cell therapy in the subject, e.g., as measured by an assay disclosed herein, e.g., as measured by the copy number of CAR transgenes per μg DNA using qPCR.

In some embodiments, the value for the level or activity of CD45RO−CD27+CD8+ cells comprises the percentage of CD45RO−CD27+CD8+ cells among CD8+ T cells, e.g., as measured by an assay disclosed herein, e.g., flow cytometry.

In some embodiments, the percentage of CD45RO−CD27+CD8+ cells among CD8+ T cells being greater than or equal to 20% (e.g., between 20% and 90%, e.g., between 20% and 80%, e.g., between 20% and 70%, e.g., between 20% and 60%) is indicative or predictive of one, two, three, or all of:

(a) increased responsiveness of the subject to the BCMA CAR-expressing cell therapy;

(b) the subject as a responder of the BCMA CAR-expressing cell therapy;

(c) the subject as suitable for the BCMA CAR-expressing cell therapy; or

(d) increased expansion of the BCMA CAR-expressing cell therapy in the subject, e.g., as measured by an assay disclosed herein, e.g., as measured by the copy number of CAR transgenes per μg DNA using qPCR.

In some embodiments, the percentage of CD45RO−CD27+CD8+ cells among CD8+ T cells being less than 20% (e.g., between 0.1% and 20%, e.g., between 0.1% and 18%, e.g., between 0.1% and 15%, e.g., between 0.1% and 12%, e.g., between 0.1% and 10%) is indicative or predictive of one, two, or all of:

(a) decreased responsiveness of the subject to the BCMA CAR-expressing cell therapy;

(b) the subject as a non-responder of the BCMA CAR-expressing cell therapy; or

(c) decreased expansion of the BCMA CAR-expressing cell therapy in the subject, e.g., as measured by an assay disclosed herein, e.g., as measured by the copy number of CAR transgenes per μg DNA using qPCR.

In some embodiments, the value for the level or activity of CCR7+CD45RO−CD27+CD8+ cells comprises the percentage of CCR7+CD45RO−CD27+CD8+ cells among CD8+ T cells, e.g., as measured by an assay disclosed herein, e.g., flow cytometry.

In some embodiments, the percentage of CCR7+CD45RO−CD27+CD8+ cells among CD8+ T cells being greater than or equal to 15% (e.g., between 15% and 90%, e.g., between 15% and 80%, e.g., between 15% and 70%, e.g., between 15% and 60%, e.g., between 15% and 50%) is indicative or predictive of one, two, three, or all of:

(a) increased responsiveness of the subject to the BCMA CAR-expressing cell therapy;

(b) the subject as a responder of the BCMA CAR-expressing cell therapy;

(c) the subject as suitable for the BCMA CAR-expressing cell therapy; or

(d) increased expansion of the BCMA CAR-expressing cell therapy in the subject, e.g., as measured by an assay disclosed herein, e.g., as measured by the copy number of CAR transgenes per μg DNA using qPCR.

In some embodiments, the percentage of CCR7+CD45RO−CD27+CD8+ cells among CD8+ T cells being less than 15% (e.g., between 0.1% and 15%, e.g., between 0.1% and 12%, e.g., between 0.1% and 10%, e.g., between 0.1% and 8%) is indicative or predictive of one, two, or all of:

(a) decreased responsiveness of the subject to the BCMA CAR-expressing cell therapy;

(b) the subject as a non-responder of the BCMA CAR-expressing cell therapy; or

(c) decreased expansion of the BCMA CAR-expressing cell therapy in the subject, e.g., as measured by an assay disclosed herein, e.g., as measured by the copy number of CAR transgenes per μg DNA using qPCR.

In some embodiments, the value for the proliferation of seeded cells from the subject during manufacturing of the BCMA CAR-expressing cell therapy comprises the fold expansion of seeded cells from the subject during manufacturing (e.g., total cell counts at the end of manufacturing relative to at the start of manufacturing) of the BCMA CAR-expressing cell therapy, e.g., as measured by an assay disclosed herein, e.g., as measured by cell counting.

In some embodiments, the method further comprises performing:

manufacturing the BCMA CAR-expressing cell therapy using cells (e.g., T cells) from the subject and administering the BCMA CAR-expressing cell therapy to the subject; or

administering, e.g., initiating administering or continuing administering, the BCMA CAR-expressing cell therapy to the subject, when:

(a) the subject was indicated or predicted to have increased responsiveness to the BCMA CAR-expressing cell therapy;

(b) the subject was indicated or predicted as a responder of the BCMA CAR-expressing cell therapy;

(c) the subject was indicated or predicted as suitable for the BCMA CAR-expressing cell therapy; or

(d) the BCMA CAR-expressing cell therapy was indicated or predicted to have increased expansion in the subject, e.g., as measured by an assay disclosed herein, e.g., as measured by the copy number of CAR transgenes per μg DNA using qPCR.

In some embodiments, the method further comprises performing one, two, three, four, five, six, seven, or all of:

administering an altered dosing regimen of the BCMA CAR-expressing cell therapy (e.g., a dosing regimen with a higher dose and/or more frequent administration than a reference dosing regimen) to the subject;

administering a second therapy (e.g., a second therapy that is not the BCMA CAR-expressing cell therapy) to the subject;

administering the BCMA CAR-expressing cell therapy and a second therapy to the subject;

discontinuing administration of the BCMA CAR-expressing cell therapy and optionally administering a second therapy to the subject;

modifying a manufacturing process of the BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;

modifying a manufacturing process of the BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;

modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or

administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject, when:

(a) the subject was indicated or predicted to have decreased responsiveness to the BCMA CAR-expressing cell therapy;

(b) the subject was indicated or predicted as a non-responder of the BCMA CAR-expressing cell therapy; or

(c) the BCMA CAR-expressing cell therapy was indicated or predicted to have decreased expansion in the subject, e.g., as measured by an assay disclosed herein, e.g., as measured by the copy number of CAR transgenes per μg DNA using qPCR.

In one aspect, disclosed herein is a method of treating a subject having a disease associated with the expression of BCMA, comprising:

responsive to an increased value for one, two, three, four, five, or all of:

(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,

(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),

(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),

(iv) the level or activity of CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),

(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)), or

(vi) the proliferation of seeded cells from the subject during manufacturing of a BCMA CAR-expressing cell therapy,

as compared to a reference value, e.g., a non-responder reference value, performing:

manufacturing a BCMA CAR-expressing cell therapy using cells (e.g., T cells) from the subject and administering the BCMA CAR-expressing cell therapy to the subject; or

administering, e.g., initiating administering or continuing administering, a BCMA CAR-expressing cell therapy to the subject,

thereby treating the subject having the disease associated with the expression of BCMA.

In some embodiments, the method comprises: responsive to an increased value for one, two, three, four, five, or all of (i)-(vi), identifying or predicting one, two, three, or all of:

(a) the subject as having increased responsiveness to the BCMA CAR-expressing cell therapy;

(b) the subject as a responder of the BCMA CAR-expressing cell therapy;

(c) the subject as suitable for the BCMA CAR-expressing cell therapy; or

(d) the BCMA CAR-expressing cell therapy as having increased expansion in the subject, e.g., as measured by an assay disclosed herein, e.g., as measured by the copy number of CAR transgenes per μg DNA using qPCR.

In one aspect, disclosed herein is a method of treating a subject having a disease associated with the expression of BCMA, comprising:

responsive to a decreased value for one, two, three, four, five, or all of:

(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,

(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),

(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),

(iv) the level or activity of CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),

(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)), or

(vi) the proliferation of seeded cells from the subject during manufacturing of a BCMA CAR-expressing cell therapy,

as compared to a reference value, e.g., a responder reference value, performing one, two, three, four, five, six, seven, or all of:

administering an altered dosing regimen of a BCMA CAR-expressing cell therapy (e.g., a dosing regimen with a higher dose and/or more frequent administration than a reference dosing regimen) to the subject;

administering a second therapy (e.g., a second therapy that is not a BCMA CAR-expressing cell therapy) to the subject;

administering a BCMA CAR-expressing cell therapy and a second therapy to the subject;

discontinuing administration of a BCMA CAR-expressing cell therapy and optionally administering a second therapy to the subject;

modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;

modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;

modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or

administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject,

thereby treating the subject having the disease associated with the expression of BCMA.

In some embodiments, the method comprises: response to a decreased value for one, two, three, four, five, or all of (i)-(vi), identifying or predicting one, two, or all of:

(a) the subject as having decreased responsiveness to the BCMA CAR-expressing cell therapy;

(b) the subject as a non-responder of the BCMA CAR-expressing cell therapy; or

(c) the BCMA CAR-expressing cell therapy as having decreased expansion in the subject, e.g., as measured by an assay disclosed herein, e.g., as measured by the copy number of CAR transgenes per μg DNA using qPCR.

In some embodiments, the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry.

In some embodiments, the method comprises:

responsive to the ratio being:

(1) greater than or equal to 1 (e.g., between 1 and 5, e.g., between 1 and 3.5); or

(2) greater than or equal to 1.6 (e.g., between 1.6 and 5, e.g., between 1.6 and 3.5), performing:

manufacturing a BCMA CAR-expressing cell therapy using cells (e.g., T cells) from the subject and administering the BCMA CAR-expressing cell therapy to the subject; or

administering, e.g., initiating administering or continuing administering, a BCMA CAR-expressing cell therapy to the subject.

In some embodiments, the method comprises:

responsive to the ratio being less than 1 (e.g., between 0.001 and 1), performing one, two, three, four, five, six, seven, or all of:

administering an altered dosing regimen of a BCMA CAR-expressing cell therapy (e.g., a dosing regimen with a higher dose and/or more frequent administration than a reference dosing regimen) to the subject;

administering a second therapy (e.g., a second therapy that is not a BCMA CAR-expressing cell therapy) to the subject;

administering a BCMA CAR-expressing cell therapy and a second therapy to the subject;

discontinuing administration of a BCMA CAR-expressing cell therapy and optionally administering a second therapy to the subject;

modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;

modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;

modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or

administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject.

In some embodiments, the value for the level or activity of CD8+ Tscm (stem cell memory T cells) comprises the percentage of CD8+ Tscm (stem cell memory T cells) among CD8+ T cells, e.g., as measured by an assay disclosed herein, e.g., flow cytometry.

In some embodiments, the value for the level or activity of HLADR-CD95+CD27+CD8+ cells comprises the percentage of HLADR-CD95+CD27+CD8+ cells among CD8+ T cells, e.g., as measured by an assay disclosed herein, e.g., flow cytometry.

In some embodiments, the method comprises:

responsive to the percentage of HLADR-CD95+CD27+CD8+ cells among CD8+ T cells being greater than or equal to 25% (e.g., between 30% and 90%, e.g., between 35% and 85%, e.g., between 40% and 80%, e.g., between 45% and 75%, e.g., between 50% and 75%), performing:

manufacturing a BCMA CAR-expressing cell therapy using cells (e.g., T cells) from the subject and administering the BCMA CAR-expressing cell therapy to the subject; or

administering, e.g., initiating administering or continuing administering, a BCMA CAR-expressing cell therapy to the subject.

In some embodiments, the method comprises:

responsive to the percentage of HLADR-CD95+CD27+CD8+ cells among CD8+ T cells being less than 25% (e.g., between 0.1% and 25%, e.g., between 0.1% and 22%, e.g., between 0.1% and 20%, e.g., between 0.1% and 18%, e.g., between 0.1% and 15%), performing one, two, three, four, five, six, seven, or all of:

administering an altered dosing regimen of a BCMA CAR-expressing cell therapy (e.g., a dosing regimen with a higher dose and/or more frequent administration than a reference dosing regimen) to the subject;

administering a second therapy (e.g., a second therapy that is not a BCMA CAR-expressing cell therapy) to the subject;

administering a BCMA CAR-expressing cell therapy and a second therapy to the subject;

discontinuing administration of a BCMA CAR-expressing cell therapy and optionally administering a second therapy to the subject;

modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;

modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;

modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or

administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject.

In some embodiments, the value for the level or activity of CD45RO−CD27+CD8+ cells comprises the percentage of CD45RO−CD27+CD8+ cells among CD8+ T cells, e.g., as measured by an assay disclosed herein, e.g., flow cytometry.

In some embodiments, the method comprises:

responsive to the percentage of CD45RO−CD27+CD8+ cells among CD8+ T cells being greater than or equal to 20% (e.g., between 20% and 90%, e.g., between 20% and 80%, e.g., between 20% and 70%, e.g., between 20% and 60%), performing:

manufacturing a BCMA CAR-expressing cell therapy using cells (e.g., T cells) from the subject and administering the BCMA CAR-expressing cell therapy to the subject; or

administering, e.g., initiating administering or continuing administering, a BCMA CAR-expressing cell therapy to the subject.

In some embodiments, the method comprises:

responsive to the percentage of CD45RO−CD27+CD8+ cells among CD8+ T cells being less than 20% (e.g., between 0.1% and 20%, e.g., between 0.1% and 18%, e.g., between 0.1% and 15%, e.g., between 0.1% and 12%, e.g., between 0.1% and 10%), performing one, two, three, four, five, six, seven, or all of:

administering an altered dosing regimen of a BCMA CAR-expressing cell therapy (e.g., a dosing regimen with a higher dose and/or more frequent administration than a reference dosing regimen) to the subject;

administering a second therapy (e.g., a second therapy that is not a BCMA CAR-expressing cell therapy) to the subject;

administering a BCMA CAR-expressing cell therapy and a second therapy to the subject;

discontinuing administration of a BCMA CAR-expressing cell therapy and optionally administering a second therapy to the subject;

modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;

modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;

modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or

administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject.

In some embodiments, the value for the level or activity of CCR7+CD45RO−CD27+CD8+ cells comprises the percentage of CCR7+CD45RO−CD27+CD8+ cells among CD8+ T cells, e.g., as measured by an assay disclosed herein, e.g., flow cytometry.

In some embodiments, the method comprises:

responsive to the percentage of CCR7+CD45RO−CD27+CD8+ cells among CD8+ T cells being greater than or equal to 15% (e.g., between 15% and 90%, e.g., between 15% and 80%, e.g., between 15% and 70%, e.g., between 15% and 60%, e.g., between 15% and 50%), performing:

manufacturing a BCMA CAR-expressing cell therapy using cells (e.g., T cells) from the subject and administering the BCMA CAR-expressing cell therapy to the subject; or

administering, e.g., initiating administering or continuing administering, a BCMA CAR-expressing cell therapy to the subject.

In some embodiments, the method comprises:

responsive to the percentage of CCR7+CD45RO−CD27+CD8+ cells among CD8+ T cells being less than 15% (e.g., between 0.1% and 15%, e.g., between 0.1% and 12%, e.g., between 0.1% and 10%, e.g., between 0.1% and 8%), performing one, two, three, four, five, six, seven, or all of:

administering an altered dosing regimen of a BCMA CAR-expressing cell therapy (e.g., a dosing regimen with a higher dose and/or more frequent administration than a reference dosing regimen) to the subject;

administering a second therapy (e.g., a second therapy that is not a BCMA CAR-expressing cell therapy) to the subject;

administering a BCMA CAR-expressing cell therapy and a second therapy to the subject;

discontinuing administration of a BCMA CAR-expressing cell therapy and optionally administering a second therapy to the subject;

modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;

modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;

modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or

administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject.

In some embodiments, the value for the proliferation of seeded cells from the subject during manufacturing of the BCMA CAR-expressing cell therapy comprises the fold expansion of seeded cells from the subject during manufacturing (e.g., total cell counts at the end of manufacturing relative to at the start of manufacturing) of the BCMA CAR-expressing cell therapy, e.g., as measured by an assay disclosed herein, e.g., as measured by cell counting.

In one aspect, provided herein is a method of evaluating or predicting the potency of a BCMA CAR-expressing cell therapy in a subject, wherein the subject has a disease associated with the expression of BCMA and wherein the BCMA CAR-expressing cell therapy is manufactured using cells (e.g., T cells) from the subject, comprising:

acquiring a value for one, two, three, four, five, or all of:

(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,

(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(iv) the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)), or

(vi) the proliferation of seeded cells from the subject during manufacturing of the BCMA CAR-expressing cell therapy, e.g., as measured by population doublings by day 9 (PDL9), wherein:

(a) an increase in the value of one, two, three, four, five, or all of (i)-(vi), as compared to a reference value, e.g., a non-responder reference value, is indicative or predictive of increased potency of the BCMA CAR-expressing cell therapy in the subject; or

(b) a decrease in the value of one, two, three, four, five, or all of (i)-(vi), as compared to a reference value, e.g., a responder reference value, is indicative or predictive of decreased potency of the BCMA CAR-expressing cell therapy in the subject,

thereby evaluating or predicting the potency of the BCMA CAR-expressing cell therapy.

In some embodiments, the method comprises acquiring a value for the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy. In some embodiments, the method comprises acquiring a value for the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the proliferation of seeded cells from the subject during manufacturing of the BCMA CAR-expressing cell therapy, e.g., population doublings by day 9 (PDL9).

In some embodiments, the increase in the value of one, two, three, four, five, or all of (i)-(vi), as compared to a reference value, e.g., a non-responder reference value, is indicative or predictive of increased expansion of the BCMA CAR-expressing cell therapy in the subject, e.g., as measured by an assay disclosed herein, e.g., as measured by the copy number of CAR transgenes per μg DNA using qPCR.

In some embodiments, the decrease in the value of one, two, three, four, five, or all of (i)-(vi), as compared to a reference value, e.g., a responder reference value, is indicative or predictive of decreased expansion of the BCMA CAR-expressing cell therapy in the subject, e.g., as measured by an assay disclosed herein, e.g., as measured by the copy number of CAR transgenes per μg DNA using qPCR.

In one aspect, disclosed herein is a method of manufacturing a BCMA CAR-expressing cell therapy, wherein the BCMA CAR-expressing cell therapy is manufactured using cells (e.g., T cells) from a subject, comprising:

acquiring a value for one, two, three, four, five, or all of:

(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,

(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(iv) the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)), or

(vi) the proliferation of seeded cells from the subject during manufacturing of the BCMA CAR-expressing cell therapy, e.g., as measured by population doublings by day 9 (PDL9), wherein: responsive to an increase in the value of one, two, three, four, five, or all of (i)-(vi), as compared to a reference value, e.g., a non-responder reference value, manufacturing the BCMA CAR-expressing cell therapy using cells from the subject.

In some embodiments, the method comprises acquiring a value for the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy. In some embodiments, the method comprises acquiring a value for the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the proliferation of seeded cells from the subject during manufacturing of the BCMA CAR-expressing cell therapy, e.g., population doublings by day 9 (PDL9).

In one aspect, provided herein is a method of manufacturing a BCMA CAR-expressing cell therapy, wherein the BCMA CAR-expressing cell therapy is manufactured using cells (e.g., T cells) from a subject, comprising:

acquiring a value for one, two, three, four, five, or all of:

(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,

(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(iv) the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),

(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)), or

(vi) the proliferation of seeded cells from the subject during manufacturing of the BCMA CAR-expressing cell therapy, e.g., as measured by population doublings by day 9 (PDL9), wherein:

responsive to a decrease in the value of one, two, three, four, five, or all of (i)-(vi), as compared to a reference value, e.g., a responder reference value, performing one, two, three, or all of:

modifying a manufacturing process of the BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR;

modifying a manufacturing process of the BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR;

modifying a manufacturing process of the BCMA CAR-expressing cell therapy, e.g., increasing the proliferation of seeded cells from the subject during the manufacturing of the BCMA CAR-expressing cell therapy; or

administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), and manufacturing the BCMA CAR-expressing cell therapy using cells (e.g., T cells) from the subject.

In some embodiments, the method comprises acquiring a value for the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy. In some embodiments, the method comprises acquiring a value for the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)). In some embodiments, the method comprises acquiring a value for the proliferation of seeded cells from the subject during manufacturing of the BCMA CAR-expressing cell therapy, e.g., population doublings by day 9 (PDL9).

In some embodiments, the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry.

In some embodiments, the value for the level or activity of CD8+ Tscm (stem cell memory T cells) comprises the percentage of CD8+ Tscm (stem cell memory T cells) among CD8+ T cells, e.g., as measured by an assay disclosed herein, e.g., flow cytometry.

In some embodiments, the value for the level or activity of HLADR-CD95+CD27+CD8+ cells comprises the percentage of HLADR-CD95+CD27+CD8+ cells among CD8+ T cells, e.g., as measured by an assay disclosed herein, e.g., flow cytometry.

In some embodiments, the value for the level or activity of CD45RO−CD27+CD8+ cells comprises the percentage of CD45RO−CD27+CD8+ cells among CD8+ T cells, e.g., as measured by an assay disclosed herein, e.g., flow cytometry.

In some embodiments, the value for the level or activity of CCR7+CD45RO−CD27+CD8+ cells comprises the percentage of CCR7+CD45RO−CD27+CD8+ cells among CD8+ T cells, e.g., as measured by an assay disclosed herein, e.g., flow cytometry.

In some embodiments, the value for the proliferation of seeded cells from the subject during manufacturing of the BCMA CAR-expressing cell therapy comprises the fold expansion of seeded cells from the subject during manufacturing (e.g., total cell counts at the end of manufacturing relative to at the start of manufacturing) of the BCMA CAR-expressing cell therapy, e.g., as measured by an assay disclosed herein, e.g., as measured by cell counting.

In certain embodiments of the foregoing aspects, the method comprises administering the BCMA CAR-expressing cell therapy to the subject in combination with one, two, or all of:

(1) an agent that increases the efficacy of the cell comprising the CAR nucleic acid or CAR polypeptide;

(2) an agent that ameliorates one or more side effects associated with administration of the cell comprising the CAR nucleic acid or CAR polypeptide;

(3) an agent that treats the disease associated with the expression of BCMA.

In certain embodiments of the foregoing aspects, the method comprises administering the BCMA CAR-expressing cell therapy to the subject in combination with a compound of Formula (I) (COF1), wherein the COF1 is:

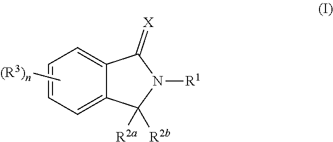

or a pharmaceutically acceptable salt, ester, hydrate, solvate, or tautomer thereof, wherein:

X is O or S;

R¹ is C₁-C₆ alkyl, C₂-C₆ alkenyl, C₂-C₆ alkynyl, C₁-C₆ heteroalkyl, carbocyclyl, heterocyclyl, aryl, or heteroaryl, each of which is optionally substituted by one or more R⁴;

each of R^{2a} and R^{2b} is independently hydrogen or C₁-C₆ alkyl; or R^{2a} and R^{2b} together with the carbon atom to which they are attached form a carbonyl group or a thiocarbonyl group;

each of R³ is independently C₁-C₆ alkyl, C₂-C₆ alkenyl, C₂-C₆ alkynyl, C₁-C₆ heteroalkyl, halo, cyano, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓ(R^{E}, —S(O)ₓN(R^{C})(R^{D}), or —N(R^{C})S(O)ₓR^{E}, wherein each alkyl, alkenyl, alkynyl, and heteroalkyl is independently and optionally substituted with one or more R⁶;

each R⁴ is independently C₁-C₆ alkyl, C₂-C₆ alkenyl, C₂-C₆ alkynyl, C₁-C₆ heteroalkyl, halo, cyano, oxo, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)ₓN(R^{C})(R^{D}), —N(R^{C})S(O)ₓR^{E}, carbocyclyl, heterocyclyl, aryl, or heteroaryl, wherein each alkyl, alkenyl, alkynyl, heteroalkyl, carbocyclyl, heterocyclyl, aryl, and heteroaryl is independently and optionally substituted with one or more R⁷;

each of R^{A}, R^{B}, R^{C}, R^{D}, and R^{E} is independently hydrogen or C₁-C₆ alkyl;

each R⁶ is independently C₁-C₆ alkyl, oxo, cyano, —OR^{B}, —N(R^{C})(R^{D}), —C(O)N(R^{C})(R^{D}), —N(R^{C})C(O)R^{A}, aryl, or heteroaryl, wherein each aryl and heteroaryl is independently and optionally substituted with one or more R⁸;

each R⁷ is independently halo, oxo, cyano, —OR^{B}, —N(R^{C})(R^{D}), —C(O)N(R^{C})(R^{D}), or —N(R^{C})C(O)R^{A};

each R⁸ is independently C₁-C₆ alkyl, cyano, —OR^{B}, —N(R^{C})(R^{D}), —C(O)N(R^{C})(R^{D}), or —N(R^{C})C(O)R^{A};

n is 0, 1, 2, 3 or 4; and

x is 0, 1, or 2, optionally wherein:

(1) the COF1 is an immunomodulatory imide drug (IMiD), or a pharmaceutically acceptable salt thereof;

(2) the COF1 is selected from the group consisting of lenalidomide, pomalidomide, thalidomide, and 2-(4-(tert-butyl)phenyl)-N-((2-(2,6-dioxopiperidin-3-yl)-1-oxoisoindolin-5-yl)methyl)acetamide, or a pharmaceutically acceptable salt thereof;

(3) the COF1 is selected from the group consisting of:

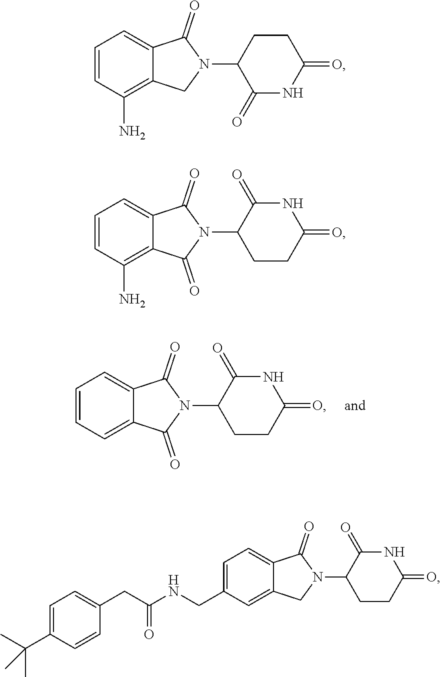 [SMILES: Nc1cccc2c1CN(C1CCC(=O)NC1=O)C2=O ; Nc1cccc2c1C(=O)N(C1CCC(=O)NC1=O)C2=O ; O=C1CCC(N2C(=O)c3ccccc3C2=O)C(=O)N1 ; CC(C)(C)c1ccc(CC(=O)NCc2ccc3c(c2)CN(C2CCC(=O)NC2=O)C3=O)cc1]

or a pharmaceutically acceptable salt thereof; or

(4) the COF1 is lenalidomide, or a pharmaceutically acceptable salt thereof.

In certain embodiments of the foregoing aspects, the method comprises administering the BCMA CAR-expressing cell therapy to the subject in combination with a kinase inhibitor, e.g., a BTK inhibitor, e.g., ibrutinib.

In certain embodiments of the foregoing aspects, the method comprises administering the BCMA CAR-expressing cell therapy to the subject in combination with a second CAR-expressing cell therapy.

In some embodiments, the second CAR-expressing cell therapy is a CD19 CAR-expressing cell therapy, e.g., a CD19 CAR-expressing cell therapy disclosed herein, e.g., CTL119 or CTL019. In some embodiments, the CD19 CAR-expressing cell therapy is administered after the administration of the BCMA CAR-expressing cell therapy, e.g., after CD19 expression is increased in the subject following the administration of the BCMA CAR-expressing cell therapy. In some embodiments, the CD19 CAR-expressing cell therapy comprises a cell (e.g., a population of cells) expressing a CD19 CAR. In some embodiments, the CD19 CAR comprises an amino acid sequence disclosed in Table 8, 9, or 10 (e.g., a CDR, scFv, or full-length amino acid sequence disclosed in Table 8, 9, or 10), or a sequence at least about 85%, 90%, 95%, 99% or more identical thereto, and/or having one, two, three or more substitutions, insertions or deletions, e.g., conserved substitutions.

In some embodiments, the second CAR-expressing cell therapy is a CD20 CAR-expressing cell therapy, e.g., a CD20 CAR-expressing cell therapy disclosed herein. In some embodiments, the CD20 CAR-expressing cell therapy is administered after the administration of the BCMA CAR-expressing cell therapy, e.g., after CD20 expression is increased in the subject following the administration of the BCMA CAR-expressing cell therapy. In some embodiments, the CD20 CAR-expressing cell therapy comprises a cell (e.g., a population of cells) expressing a CD20 CAR. In some embodiments, the CD20 CAR comprises an amino acid sequence disclosed in Table 11, 12, or 13 (e.g., a CDR, scFv, or full-length amino acid sequence disclosed in Table 11, 12, or 13), or a sequence at least about 85%, 90%, 95%, 99% or more identical thereto, and/or having one, two, three or more substitutions, insertions or deletions, e.g., conserved substitutions.

In some embodiments, the second CAR-expressing cell therapy is a CD22 CAR-expressing cell therapy, e.g., a CD22 CAR-expressing cell therapy disclosed herein. In some embodiments, the CD22 CAR-expressing cell therapy is administered after the administration of the BCMA CAR-expressing cell therapy, e.g., after CD22 expression is increased in the subject following the administration of the BCMA CAR-expressing cell therapy. In some embodiments, the CD22 CAR-expressing cell therapy comprises a cell (e.g., a population of cells) expressing a CD22 CAR. In some embodiments, the CD22 CAR comprises an amino acid sequence disclosed in Table 14 or 15 (e.g., a CDR, scFv, or full-length amino acid sequence disclosed in Table 14 or 15), or a sequence at least about 85%, 90%, 95%, 99% or more identical thereto, and/or having one, two, three or more substitutions, insertions or deletions, e.g., conserved substitutions.

In some embodiments, the second CAR-expressing cell therapy is a CAR-expressing cell therapy comprising a cell expressing a first CAR and a second CAR, wherein the first CAR is a BCMA CAR (e.g., a BCMA CAR disclosed herein). In some embodiments, the second CAR is selected from the group consisting of a CD19 CAR (e.g., a CD19 CAR disclosed herein), a CD20 CAR (e.g., a CD20 CAR disclosed herein), and a CD22 CAR (e.g., a CD22 CAR disclosed herein). In some embodiments, the second CAR is a CD19 CAR (e.g., a CD19 CAR disclosed herein). In some embodiments, the CD19 CAR-expressing cell therapy comprises a cell (e.g., a population of cells) expressing a CD19 CAR. In some embodiments, the CD19 CAR comprises an amino acid sequence disclosed in Table 8, 9, or 10 (e.g., a CDR, scFv, or full-length amino acid sequence disclosed in Table 8, 9, or 10), or a sequence at least about 85%, 90%, 95%, 99% or more identical thereto, and/or having one, two, three or more substitutions, insertions or deletions, e.g., conserved substitutions. In some embodiments, the second CAR is a CD20 CAR (e.g., a CD20 CAR disclosed herein). In some embodiments, the CD20 CAR comprises an amino acid sequence disclosed in Table 11, 12, or 13 (e.g., a CDR, scFv, or full-length amino acid sequence disclosed in Table 11, 12, or 13), or a sequence at least about 85%, 90%, 95%, 99% or more identical thereto, and/or having one, two, three or more substitutions, insertions or deletions, e.g., conserved substitutions. In some embodiments, the second CAR is a CD22 CAR (e.g., a CD22 CAR disclosed herein). In some embodiments, the CD22 CAR comprises an amino acid sequence disclosed in Table 14 or 15 (e.g., a CDR, scFv, or full-length amino acid sequence disclosed in Table 14 or 15), or a sequence at least about 85%, 90%, 95%, 99% or more identical thereto, and/or having one, two, three or more substitutions, insertions or deletions, e.g., conserved substitutions.

In some embodiments, the second CAR-expressing cell therapy is a CAR-expressing cell therapy comprising a cell expressing a multispecific CAR, e.g., a bispecific CAR, that binds to a first antigen and a second antigen, wherein the first antigen is BCMA. In some embodiments, the second antigen is selected from the group consisting of CD19, CD20, and CD22. In some embodiments, the second antigen is CD19. In some embodiments, the second antigen is CD20. In some embodiments, the second antigen is CD22.

In certain embodiments of the foregoing aspects, the method comprises administering the BCMA CAR-expressing cell therapy to the subject in combination with a CD19 inhibitor, e.g., a CD19 inhibitor disclosed herein. In some embodiments, the CD19 inhibitor is administered after the administration of the BCMA CAR-expressing cell therapy, e.g., after CD19 expression is increased in the subject following the administration of the BCMA CAR-expressing cell therapy.

In certain embodiments of the foregoing aspects, the method comprises administering the BCMA CAR-expressing cell therapy to the subject in combination with a CD20 inhibitor, e.g., a CD20 inhibitor disclosed herein. In some embodiments, the CD20 inhibitor is a multispecific antibody molecule, e.g., a bispecific antibody molecule, that binds to CD20 and CD3, e.g., THG338. In some embodiments, the CD20 inhibitor is administered after the administration of the BCMA CAR-expressing cell therapy, e.g., after CD20 expression is increased in the subject following the administration of the BCMA CAR-expressing cell therapy.

In certain embodiments of the foregoing aspects, the method comprises administering the BCMA CAR-expressing cell therapy to the subject in combination with a CD22 inhibitor, e.g., a CD22 inhibitor disclosed herein. In some embodiments, the CD22 inhibitor is administered after the administration of the BCMA CAR-expressing cell therapy, e.g., after CD22 expression is increased in the subject following the administration of the BCMA CAR-expressing cell therapy.

In certain embodiments of the foregoing aspects, the method comprises administering the BCMA CAR-expressing cell therapy to the subject in combination with a molecule that binds to Fc receptor like 2 (FCRL2) or Fc receptor like 5 (FCRL5). In some embodiments, the molecule is a CAR-expressing cell therapy comprising a cell expressing a CAR that binds to FCRL2 or FCRL5. In some embodiments, the molecule is a multispecific antibody molecule, e.g., a bispecific antibody molecule, that binds to a first antigen and a second antigen, wherein the first antigen is FCRL2 or FCRL5, optionally wherein the second antigen is CD3.

In certain embodiments of the foregoing aspects, the method comprises administering the BCMA CAR-expressing cell therapy to the subject in combination with an interleukin-15 (IL-15) polypeptide, an interleukin-15 receptor alpha (IL-15Ra) polypeptide, or a combination of both an IL-15 polypeptide and an IL-15Ra polypeptide, e.g., hetIL-15.

In certain embodiments of the foregoing aspects, the method comprises administering the BCMA CAR-expressing cell therapy to the subject in combination with an inhibitor of TGF beta.

In certain embodiments of the foregoing aspects, the method comprises administering the BCMA CAR-expressing cell therapy to the subject in combination with an EGFR inhibitor, e.g., an EGFR^{mut}-tyrosine kinase inhibitor (TKI). In some embodiments, the EGFR inhibitor is EGF816. In some embodiments, the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide. In some embodiments, the EGFR inhibitor is compound A40 disclosed in Table 27.

In certain embodiments of the foregoing aspects, the method comprises administering the BCMA CAR-expressing cell therapy to the subject in combination with an adenosine A2AR antagonist. In some embodiments, the adenosine A2AR antagonist is selected from the group consisting of PBF509, CPI444, AZD4635, Vipadenant, GBV-2034, and AB928. In some embodiments, the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine

In certain embodiments of the foregoing aspects, the method comprises administering the BCMA CAR-expressing cell therapy to the subject in combination with an anti-CD73 antibody molecule, e.g., an anti-CD73 antibody molecule disclosed herein.

In certain embodiments of the foregoing aspects, the method comprises administering the BCMA CAR-expressing cell therapy to the subject in combination with a check point inhibitor. In some embodiments, the check point inhibitor is a PD-1 inhibitor. In some embodiments, the PD-1 inhibitor is selected from the group consisting of PDR001, Nivolumab, Pembrolizumab, Pidilizumab, MEDI0680, REGN2810, TSR-042, PF-06801591, and AMP-224. In some embodiments, the PD-1 inhibitor increases expansion of BCMA CAR-expressing cells in the subject, e.g. for at least 1, 2, 3, 4, or 5 weeks, e.g., for at least, 1, 2, 3, 4, 5, 10, 15, 20, 25, or 30-fold. In some embodiments, the BCMA CAR-expressing cells are administered to the subject prior to the administration of the PD-1 inhibitor. In some embodiments, the BCMA CAR-expressing cells do not expand or have minimal expansion (e.g., no more than 1, 2, 3, 4, 5, or 10-fold expansion) in the subject at the time the PD-1 inhibitor is administered. In some embodiments, the check point inhibitor is a PD-L1 inhibitor. In some embodiments, the PD-L1 inhibitor is selected from the group consisting of FAZ053, Atezolizumab, Avelumab, Durvalumab, and BMS-936559. In some embodiments, the PD-L1 inhibitor increases expansion of BCMA CAR-expressing cells in the subject, e.g. for at least 1, 2, 3, 4, or 5 weeks, e.g., for at least, 1, 2, 3, 4, 5, 10, 15, 20, 25, or 30-fold. In some embodiments, the BCMA CAR-expressing cells are administered to the subject prior to the administration of the PD-L1 inhibitor. In some embodiments, the BCMA CAR-expressing cells do not expand or have minimal expansion (e.g., no more than 1, 2, 3, 4, 5, or 10-fold expansion) in the subject at the time the PD-L1 inhibitor is administered. In some embodiments, the check point inhibitor is a LAG-3 inhibitor. In some embodiments, the LAG-3 inhibitor is selected from the group consisting of LAG525, BMS-986016, TSR-033, MK-4280 and REGN3767. In some embodiments, the check point inhibitor is a TIM-3 inhibitor. In some embodiments, the TIM-3 inhibitor is selected from the group consisting of MGB453, TSR-022, and LY3321367.

In certain embodiments of the foregoing aspects, the method comprises administering the BCMA CAR-expressing cell therapy to the subject in combination with an antibody molecule that binds to CD32B.

In certain embodiments of the foregoing aspects, the method comprises administering the BCMA CAR-expressing cell therapy to the subject in combination with an antibody molecule that binds to IL-17, e.g., an antagonistic antibody molecule that binds to IL-17, e.g., CJM112.

In certain embodiments of the foregoing aspects, the method comprises administering the BCMA CAR-expressing cell therapy to the subject in combination with an antibody molecule that binds to IL-1 beta.

In certain embodiments of the foregoing aspects, the method comprises administering the BCMA CAR-expressing cell therapy to the subject in combination with an inhibitor of indoleamine 2,3-dioxygenase (IDO) and/or tryptophan 2,3-dioxygenase (TDO), e.g., an IDO1 inhibitor. In some embodiments, the inhibitor of IDO and/or TDO is INCB24360, indoximod, NLG919, epacadostat, NLG919, or F001287. In some embodiments, the inhibitor of IDO and/or TDO is (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan.

In one aspect, disclosed herein is a method of treating a subject having a disease associated with the expression of BCMA, comprising administering a BCMA CAR-expressing cell therapy and a second therapy to the subject. In some embodiments, the second therapy is a CD19 CAR-expressing cell therapy, e.g., a CD19 CAR-expressing cell therapy disclosed herein, e.g., CTL119 or CTL019. In some embodiments, the CD19 CAR-expressing cell therapy is administered after the administration of the BCMA CAR-expressing cell therapy, e.g., after CD19 expression is increased in the subject following the administration of the BCMA CAR-expressing cell therapy. In some embodiments, the CD19 CAR-expressing cell therapy comprises a cell (e.g., a population of cells) expressing a CD19 CAR. In some embodiments, the CD19 CAR comprises an amino acid sequence disclosed in Table 8, 9, or 10 (e.g., a CDR, scFv, or full-length amino acid sequence disclosed in Table 8, 9, or 10), or a sequence at least about 85%, 90%, 95%, 99% or more identical thereto, and/or having one, two, three or more substitutions, insertions or deletions, e.g., conserved substitutions.

In some embodiments, the second therapy is a CD20 CAR-expressing cell therapy, e.g., a CD20 CAR-expressing cell therapy disclosed herein. In some embodiments, the CD20 CAR-expressing cell therapy is administered after the administration of the BCMA CAR-expressing cell therapy, e.g., after CD20 expression is increased in the subject following the administration of the BCMA CAR-expressing cell therapy. In some embodiments, the CD20 CAR-expressing cell therapy comprises a cell (e.g., a population of cells) expressing a CD20 CAR. In some embodiments, the CD20 CAR comprises an amino acid sequence disclosed in Table 11, 12, or 13 (e.g., a CDR, scFv, or full-length amino acid sequence disclosed in Table 11, 12, or 13), or a sequence at least about 85%, 90%, 95%, 99% or more identical thereto, and/or having one, two, three or more substitutions, insertions or deletions, e.g., conserved substitutions.

In some embodiments, the second therapy is a CD22 CAR-expressing cell therapy, e.g., a CD22 CAR-expressing cell therapy disclosed herein. In some embodiments, the CD22 CAR-expressing cell therapy is administered after the administration of the BCMA CAR-expressing cell therapy, e.g., after CD22 expression is increased in the subject following the administration of the BCMA CAR-expressing cell therapy. In some embodiments, the CD22 CAR-expressing cell therapy comprises a cell (e.g., a population of cells) expressing a CD22 CAR. In some embodiments, the CD22 CAR comprises an amino acid sequence disclosed in Table 14 or 15 (e.g., a CDR, scFv, or full-length amino acid sequence disclosed in Table 14 or 15), or a sequence at least about 85%, 90%, 95%, 99% or more identical thereto, and/or having one, two, three or more substitutions, insertions or deletions, e.g., conserved substitutions.

In some embodiments, the second therapy is a CAR-expressing cell therapy comprising a cell expressing a first CAR and a second CAR, wherein the first CAR is a BCMA CAR (e.g., a BCMA CAR disclosed herein). In some embodiments, the second CAR is selected from the group consisting of a CD19 CAR (e.g., a CD19 CAR disclosed herein), a CD20 CAR (e.g., a CD20 CAR disclosed herein), and a CD22 CAR (e.g., a CD22 CAR disclosed herein). In some embodiments, the second CAR is a CD19 CAR (e.g., a CD19 CAR disclosed herein). In some embodiments, the CD19 CAR-expressing cell therapy comprises a cell (e.g., a population of cells) expressing a CD19 CAR. In some embodiments, the CD19 CAR comprises an amino acid sequence disclosed in Table 8, 9, or 10 (e.g., a CDR, scFv, or full-length amino acid sequence disclosed in Table 8, 9, or 10), or a sequence at least about 85%, 90%, 95%, 99% or more identical thereto, and/or having one, two, three or more substitutions, insertions or deletions, e.g., conserved substitutions. In some embodiments, the second CAR is a CD20 CAR (e.g., a CD20 CAR disclosed herein). In some embodiments, the CD20 CAR comprises an amino acid sequence disclosed in Table 11, 12, or 13 (e.g., a CDR, scFv, or full-length amino acid sequence disclosed in Table 11, 12, or 13), or a sequence at least about 85%, 90%, 95%, 99% or more identical thereto, and/or having one, two, three or more substitutions, insertions or deletions, e.g., conserved substitutions. In some embodiments, the second CAR is a CD22 CAR (e.g., a CD22 CAR disclosed herein). In some embodiments, the CD22 CAR comprises an amino acid sequence disclosed in Table 14 or 15 (e.g., a CDR, scFv, or full-length amino acid sequence disclosed in Table 14 or 15), or a sequence at least about 85%, 90%, 95%, 99% or more identical thereto, and/or having one, two, three or more substitutions, insertions or deletions, e.g., conserved substitutions.

In some embodiments, the second therapy is a CAR-expressing cell therapy comprising a cell expressing a multispecific CAR, e.g., a bispecific CAR that binds to a first antigen and a second antigen, wherein the first antigen is BCMA. In some embodiments, the second antigen is selected from the group consisting of CD19, CD20, and CD22. In some embodiments, the second antigen is CD19. In some embodiments, the second antigen is CD20. In some embodiments, the second antigen is CD22.

In one aspect, disclosed herein is a method of treating a subject having a disease associated with the expression of BCMA, comprising administering a BCMA CAR-expressing cell therapy and a second therapy to the subject. In some embodiments, the second therapy is a CD19 inhibitor, e.g., a CD19 inhibitor disclosed herein. In some embodiments, the CD19 inhibitor is administered after the administration of the BCMA CAR-expressing cell therapy, e.g., after CD19 expression is increased in the subject following the administration of the BCMA CAR-expressing cell therapy.

In some embodiments, the second therapy is a CD20 inhibitor, e.g., a CD20 inhibitor disclosed herein. In some embodiments, the CD20 inhibitor is a multispecific antibody molecule, e.g., a bispecific antibody molecule, that binds to CD20 and CD3, e.g., THG338. In some embodiments, the CD20 inhibitor is administered after the administration of the BCMA CAR-expressing cell therapy, e.g., after CD20 expression is increased in the subject following the administration of the BCMA CAR-expressing cell therapy.

In some embodiments, the second therapy is a CD22 inhibitor, e.g., a CD22 inhibitor disclosed herein. In some embodiments, the CD22 inhibitor is administered after the administration of the BCMA CAR-expressing cell therapy, e.g., after CD22 expression is increased in the subject following the administration of the BCMA CAR-expressing cell therapy.

In one aspect, disclosed herein is a method of treating a subject having a disease associated with the expression of BCMA, comprising administering a BCMA CAR-expressing cell therapy and a second therapy to the subject, wherein the second therapy is a molecule that binds to Fc receptor like 2 (FCRL2) or Fc receptor like 5 (FCRL5). In some embodiments, the molecule is a CAR-expressing cell therapy comprising a cell expressing a CAR that binds to FCRL2 or FCRL5. In some embodiments, the molecule is a multispecific antibody molecule, e.g., a bispecific antibody molecule, that binds to a first antigen and a second antigen, wherein the first antigen is FCRL2 or FCRL5, optionally wherein the second antigen is CD3.

In one aspect, disclosed herein is a method of treating a subject having a disease associated with the expression of BCMA, comprising administering a BCMA CAR-expressing cell therapy and a second therapy to the subject, wherein the second therapy is an inhibitor of TGF beta.

In one aspect, disclosed herein is a method of treating a subject having a disease associated with the expression of BCMA, comprising administering a BCMA CAR-expressing cell therapy and a second therapy to the subject, wherein the second therapy is an EGFR inhibitor, e.g., an EGFR′-tyrosine kinase inhibitor (TKI). In some embodiments, the EGFR inhibitor is EGF816. In some embodiments, the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide. In some embodiments, the EGFR inhibitor is compound A40 disclosed in Table 27.

In one aspect, disclosed herein is a method of treating a subject having a disease associated with the expression of BCMA, comprising administering a BCMA CAR-expressing cell therapy and a second therapy to the subject, wherein the second therapy is an adenosine A2AR antagonist. In some embodiments, the adenosine A2AR antagonist is selected from the group consisting of PBF509, CPI444, AZD4635, Vipadenant, GBV-2034, and AB928. In some embodiments, the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine

In one aspect, disclosed herein is a method of treating a subject having a disease associated with the expression of BCMA, comprising administering a BCMA CAR-expressing cell therapy and a second therapy to the subject, wherein the second therapy is an anti-CD73 antibody molecule, e.g., an anti-CD73 antibody molecule disclosed herein.

In one aspect, disclosed herein is a method of treating a subject having a disease associated with the expression of BCMA, comprising administering a BCMA CAR-expressing cell therapy and a second therapy to the subject, wherein the second therapy is a check point inhibitor. In some embodiments, the check point inhibitor is a PD-1 inhibitor. In some embodiments, the PD-1 inhibitor is selected from the group consisting of PDR001, Nivolumab, Pembrolizumab, Pidilizumab, MEDI0680, REGN2810, TSR-042, PF-06801591, and AMP-224. In some embodiments, the PD-1 inhibitor is administered after the administration of the BCMA CAR-expressing cell therapy, e.g., after the expression of PD-1 or PD-L1 is increased in the subject following the administration of the BCMA CAR-expressing cell therapy. In some embodiments, the PD-1 inhibitor increases expansion of BCMA CAR-expressing cells in the subject, e.g. for at least 1, 2, 3, 4, or 5 weeks, e.g., for at least, 1, 2, 3, 4, 5, 10, 15, 20, 25, or 30-fold. In some embodiments, the BCMA CAR-expressing cells are administered to the subject prior to the administration of the PD-1 inhibitor. In some embodiments, the BCMA CAR-expressing cells do not expand or have minimal expansion (e.g., no more than 1, 2, 3, 4, 5, or 10-fold expansion) in the subject at the time the PD-1 inhibitor is administered. In some embodiments, the check point inhibitor is a PD-L1 inhibitor. In some embodiments, the PD-L1 inhibitor is selected from the group consisting of FAZ053, Atezolizumab, Avelumab, Durvalumab, and BMS-936559. In some embodiments, the PD-L1 inhibitor is administered after the administration of the BCMA CAR-expressing cell therapy, e.g., after the expression of PD-1 or PD-L1 is increased in the subject following the administration of the BCMA CAR-expressing cell therapy. In some embodiments, the PD-L1 inhibitor increases expansion of BCMA CAR-expressing cells in the subject, e.g. for at least 1, 2, 3, 4, or 5 weeks, e.g., for at least, 1, 2, 3, 4, 5, 10, 15, 20, 25, or 30-fold. In some embodiments, the BCMA CAR-expressing cells are administered to the subject prior to the administration of the PD-L1 inhibitor. In some embodiments, the BCMA CAR-expressing cells do not expand or have minimal expansion (e.g., no more than 1, 2, 3, 4, 5, or 10-fold expansion) in the subject at the time the PD-L1 inhibitor is administered. In some embodiments, the check point inhibitor is a LAG-3 inhibitor. In some embodiments, the LAG-3 inhibitor is selected from the group consisting of LAG525, BMS-986016, TSR-033, MK-4280 and REGN3767. In some embodiments, the LAG-3 inhibitor is administered after the administration of the BCMA CAR-expressing cell therapy, e.g., after the expression of LAG-3 is increased in the subject following the administration of the BCMA CAR-expressing cell therapy. In some embodiments, the check point inhibitor is a TIM-3 inhibitor. In some embodiments, the TIM-3 inhibitor is selected from the group consisting of MGB453, TSR-022, and LY3321367. In some embodiments, the TIM-3 inhibitor is administered after the administration of the BCMA CAR-expressing cell therapy, e.g., after the expression of TIM-3 is increased in the subject following the administration of the BCMA CAR-expressing cell therapy.

In one aspect, disclosed herein is a method of treating a subject having a disease associated with the expression of BCMA, comprising administering a BCMA CAR-expressing cell therapy and a second therapy to the subject, wherein the second therapy is an antibody molecule that binds to CD32B.

In one aspect, disclosed herein is a method of treating a subject having a disease associated with the expression of BCMA, comprising administering a BCMA CAR-expressing cell therapy and a second therapy to the subject, wherein the second therapy is an antibody molecule that binds to IL-17, e.g., an antagonistic antibody molecule that binds to IL-17, e.g., CJM112.

In one aspect, disclosed herein is a method of treating a subject having a disease associated with the expression of BCMA, comprising administering a BCMA CAR-expressing cell therapy and a second therapy to the subject, wherein the second therapy is an antibody molecule that binds to IL-1 beta.

In one aspect, disclosed herein is a method of treating a subject having a disease associated with the expression of BCMA, comprising administering a BCMA CAR-expressing cell therapy and a second therapy to the subject, wherein the second therapy is an inhibitor of indoleamine 2,3-dioxygenase (IDO) and/or tryptophan 2,3-dioxygenase (TDO), e.g., an IDO1 inhibitor. In some embodiments, the inhibitor of IDO and/or TDO is INCB24360, indoximod, NLG919, epacadostat, NLG919, or F001287. In some embodiments, the inhibitor of IDO and/or TDO is (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan. In some embodiments, the inhibitor of IDO and/or TDO is administered after the administration of the BCMA CAR-expressing cell therapy, e.g., after IDO and/or TDO expression is increased in the subject following the administration of the BCMA CAR-expressing cell therapy.

In certain embodiments for the foregoing aspects, the second therapy is administered prior to, concurrently with, or subsequent to the administration of the BCMA CAR-expressing cell therapy.

In one aspect, disclosed herein is a method of treating a subject having a disease associated with the expression of BCMA, wherein the subject has received or is receiving a BCMA CAR-expressing cell therapy, comprising:

responsive to an increase in a value of the level or activity of an antigen in the subject, e.g., in a sample from the subject (e.g., a biopsy sample, e.g., a bone marrow biopsy sample), at at least one time point after the subject began receiving the BCMA CAR-expressing cell therapy, relative to a reference value, wherein the reference value is:

(i) the level or activity of the antigen in the subject, e.g., in a sample from the subject (e.g., a biopsy sample, e.g., a bone marrow biopsy sample), prior to the at least one time point (e.g., the level or activity of the antigen in the subject before the subject began receiving the BCMA CAR-expressing cell therapy, or the level or activity of the antigen in the subject after the subject began receiving the BCMA CAR-expressing cell therapy but prior to the at least one time point);

(ii) the level or activity of the antigen in a different subject having the disease associated with the expression of BCMA; or

(iii) an average level or activity of the antigen in a population of subjects having the disease associated with the expression of BCMA, administering an inhibitor of the antigen to the subject, wherein:

(1) the antigen is CD19 and the inhibitor of the antigen is a CD19 inhibitor, optionally wherein the CD19 inhibitor is:
(a) a CD19 CAR-expressing cell therapy, e.g., a CD19 CAR-expressing cell therapy disclosed herein, e.g., CTL119 or CTL019;
(b) a CAR-expressing cell therapy comprising a cell expressing a first CAR and a second CAR, wherein the first CAR is a BCMA CAR (e.g., a BCMA CAR disclosed herein) and the second CAR is a CD19 CAR (e.g., a CD19 CAR disclosed herein); or
(c) a CAR-expressing cell therapy comprising a cell expressing a multispecific CAR, e.g., a bispecific CAR, that binds to BCMA and CD19,

(2) the antigen is CD20 and the inhibitor of the antigen is a CD20 inhibitor, optionally wherein the CD20 inhibitor is:
(d) a CD20 CAR-expressing cell therapy, e.g., a CD20 CAR-expressing cell therapy disclosed herein;
(e) a CAR-expressing cell therapy comprising a cell expressing a first CAR and a second CAR, wherein the first CAR is a BCMA CAR (e.g., a BCMA CAR disclosed herein) and the second CAR is a CD20 CAR (e.g., a CD20 CAR disclosed herein);
(f) a CAR-expressing cell therapy comprising a cell expressing a multispecific CAR, e.g., a bispecific CAR, that binds to BCMA and CD20; or
(g) a multispecific antibody molecule, e.g., a bispecific antibody molecule, that binds to CD20 and CD3, e.g., THG338,

(3) the antigen is CD22 and the inhibitor of the antigen is a CD22 inhibitor, optionally wherein the CD22 inhibitor is:
(h) a CD22 CAR-expressing cell therapy, e.g., a CD22 CAR-expressing cell therapy disclosed herein;
(i) a CAR-expressing cell therapy comprising a cell expressing a first CAR and a second CAR, wherein the first CAR is a BCMA CAR (e.g., a BCMA CAR disclosed herein) and the second CAR is a CD22 CAR (e.g., a CD22 CAR disclosed herein); or
(j) a CAR-expressing cell therapy comprising a cell expressing a multispecific CAR, e.g., a bispecific CAR, that binds to BCMA and CD22,

(4) the antigen is PD1 or PD-L1 and the inhibitor of the antigen is an anti-PD1 antibody molecule or an anti-PD-L1 antibody molecule, optionally wherein the inhibitor of the antigen is:
(k) PDR001, Nivolumab, Pembrolizumab, Pidilizumab, MEDI0680, REGN2810, TSR-042, PF-06801591, or AMP-224; or
(l) FAZ053, Atezolizumab, Avelumab, Durvalumab, or BMS-936559,

(5) the antigen is IDO or TDO and the inhibitor of the antigen is an inhibitor of IDO and/or TDO, optionally wherein the inhibitor of IDO and/or TDO is:
(m) INCB24360, indoximod, NLG919, epacadostat, NLG919, or F001287; or
(n) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan, or

(6) the antigen is TGF-beta and the inhibitor of the antigen is a TGF beta inhibitor.

In one aspect, disclosed herein is a method of treating a subject having a disease associated with the expression of BCMA, wherein the subject has received or is receiving a BCMA CAR-expressing cell therapy, comprising:

acquiring a value of the level or activity of an antigen in the subject, e.g., in a sample from the subject (e.g., a biopsy sample, e.g., a bone marrow biopsy sample), at at least one time point after the subject began receiving the BCMA CAR-expressing cell therapy, responsive to an increase in the value relative to a reference value, wherein the reference value is:

(i) the level or activity of the antigen in the subject, e.g., in a sample from the subject (e.g., a biopsy sample, e.g., a bone marrow biopsy sample), prior to the at least one time point (e.g., the level or activity of the antigen in the subject before the subject began receiving the BCMA CAR-expressing cell therapy, or the level or activity of the antigen in the subject after the subject began receiving the BCMA CAR-expressing cell therapy but prior to the at least one time point);

(ii) the level or activity of the antigen in a different subject having the disease associated with the expression of BCMA; or

(iii) an average level or activity of the antigen in a population of subjects having the disease associated with the expression of BCMA,

administering an inhibitor of the antigen to the subject, wherein:

(1) the antigen is CD19 and the inhibitor of the antigen is a CD19 inhibitor, optionally wherein the CD19 inhibitor is:
(a) a CD19 CAR-expressing cell therapy, e.g., a CD19 CAR-expressing cell therapy disclosed herein, e.g., CTL119 or CTL019;
(b) a CAR-expressing cell therapy comprising a cell expressing a first CAR and a second CAR, wherein the first CAR is a BCMA CAR (e.g., a BCMA CAR disclosed herein) and the second CAR is a CD19 CAR (e.g., a CD19 CAR disclosed herein); or
(c) a CAR-expressing cell therapy comprising a cell expressing a multispecific CAR, e.g., a bispecific CAR, that binds to BCMA and CD19,

(2) the antigen is CD20 and the inhibitor of the antigen is a CD20 inhibitor, optionally wherein the CD20 inhibitor is:
(d) a CD20 CAR-expressing cell therapy, e.g., a CD20 CAR-expressing cell therapy disclosed herein;
(e) a CAR-expressing cell therapy comprising a cell expressing a first CAR and a second CAR, wherein the first CAR is a BCMA CAR (e.g., a BCMA CAR disclosed herein) and the second CAR is a CD20 CAR (e.g., a CD20 CAR disclosed herein);
(f) a CAR-expressing cell therapy comprising a cell expressing a multispecific CAR, e.g., a bispecific CAR, that binds to BCMA and CD20; or
(g) a multispecific antibody molecule, e.g., a bispecific antibody molecule, that binds to CD20 and CD3, e.g., THG338,

(3) the antigen is CD22 and the inhibitor of the antigen is a CD22 inhibitor, optionally wherein the CD22 inhibitor is:
(h) a CD22 CAR-expressing cell therapy, e.g., a CD22 CAR-expressing cell therapy disclosed herein;
(i) a CAR-expressing cell therapy comprising a cell expressing a first CAR and a second CAR, wherein the first CAR is a BCMA CAR (e.g., a BCMA CAR disclosed herein) and the second CAR is a CD22 CAR (e.g., a CD22 CAR disclosed herein); or
(j) a CAR-expressing cell therapy comprising a cell expressing a multispecific CAR, e.g., a bispecific CAR, that binds to BCMA and CD22,

(4) the antigen is PD1 or PD-L1 and the inhibitor of the antigen is an anti-PD1 antibody molecule or an anti-PD-L1 antibody molecule, optionally wherein the inhibitor of the antigen is:
(k) PDR001, Nivolumab, Pembrolizumab, Pidilizumab, MEDI0680, REGN2810, TSR-042, PF-06801591, or AMP-224; or
(l) FAZ053, Atezolizumab, Avelumab, Durvalumab, or BMS-936559,

(5) the antigen is IDO or TDO and the inhibitor of the antigen is an inhibitor of IDO and/or TDO, optionally wherein the inhibitor of IDO and/or TDO is:
(m) INCB24360, indoximod, NLG919, epacadostat, NLG919, or F001287; or
(n) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan, or

(6) the antigen is TGF-beta and the inhibitor of the antigen is a TGF beta inhibitor.

In one aspect, disclosed herein is a method of treating a subject having a disease associated with the expression of BCMA, comprising:

administering a BCMA CAR-expressing cell therapy to the subject,

responsive to an increase in a value of the level or activity of an antigen in the subject, e.g., in a sample from the subject (e.g., a biopsy sample, e.g., a bone marrow biopsy sample), at at least one time point after the subject began receiving the BCMA CAR-expressing cell therapy, relative to a reference value, wherein the reference value is:

(i) the level or activity of the antigen in the subject, e.g., in a sample from the subject (e.g., a biopsy sample, e.g., a bone marrow biopsy sample), prior to the at least one time point (e.g., the level or activity of the antigen in the subject before the subject began receiving the BCMA CAR-expressing cell therapy, or the level or activity of the antigen in the subject after the subject began receiving the BCMA CAR-expressing cell therapy but prior to the at least one time point);

(ii) the level or activity of the antigen in a different subject having the disease associated with the expression of BCMA; or

(iii) an average level or activity of the antigen in a population of subjects having the disease associated with the expression of BCMA,

administering an inhibitor of the antigen to the subject, wherein:

(1) the antigen is CD19 and the inhibitor of the antigen is a CD19 inhibitor, optionally wherein the CD19 inhibitor is:
(a) a CD19 CAR-expressing cell therapy, e.g., a CD19 CAR-expressing cell therapy disclosed herein, e.g., CTL119 or CTL019;
(b) a CAR-expressing cell therapy comprising a cell expressing a first CAR and a second CAR, wherein the first CAR is a BCMA CAR (e.g., a BCMA CAR disclosed herein) and the second CAR is a CD19 CAR (e.g., a CD19 CAR disclosed herein); or
(c) a CAR-expressing cell therapy comprising a cell expressing a multispecific CAR, e.g., a bispecific CAR, that binds to BCMA and CD19,

(2) the antigen is CD20 and the inhibitor of the antigen is a CD20 inhibitor, optionally wherein the CD20 inhibitor is:
(d) a CD20 CAR-expressing cell therapy, e.g., a CD20 CAR-expressing cell therapy disclosed herein;
(e) a CAR-expressing cell therapy comprising a cell expressing a first CAR and a second CAR, wherein the first CAR is a BCMA CAR (e.g., a BCMA CAR disclosed herein) and the second CAR is a CD20 CAR (e.g., a CD20 CAR disclosed herein);
(f) a CAR-expressing cell therapy comprising a cell expressing a multispecific CAR, e.g., a bispecific CAR, that binds to BCMA and CD20; or
(g) a multispecific antibody molecule, e.g., a bispecific antibody molecule, that binds to CD20 and CD3, e.g., THG338,

(3) the antigen is CD22 and the inhibitor of the antigen is a CD22 inhibitor, optionally wherein the CD22 inhibitor is:
(h) a CD22 CAR-expressing cell therapy, e.g., a CD22 CAR-expressing cell therapy disclosed herein;
(i) a CAR-expressing cell therapy comprising a cell expressing a first CAR and a second CAR, wherein the first CAR is a BCMA CAR (e.g., a BCMA CAR disclosed herein) and the second CAR is a CD22 CAR (e.g., a CD22 CAR disclosed herein); or
(j) a CAR-expressing cell therapy comprising a cell expressing a multispecific CAR, e.g., a bispecific CAR, that binds to BCMA and CD22,

(4) the antigen is PD1 or PD-L1 and the inhibitor of the antigen is an anti-PD1 antibody molecule or an anti-PD-L1 antibody molecule, optionally wherein the inhibitor of the antigen is:
(k) PDR001, Nivolumab, Pembrolizumab, Pidilizumab, MEDI0680, REGN2810, TSR-042, PF-06801591, or AMP-224; or
(l) FAZ053, Atezolizumab, Avelumab, Durvalumab, or BMS-936559,

(5) the antigen is IDO or TDO and the inhibitor of the antigen is an inhibitor of IDO and/or TDO, optionally wherein the inhibitor of IDO and/or TDO is:
(m) INCB24360, indoximod, NLG919, epacadostat, NLG919, or F001287; or
(n) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan, or

(6) the antigen is TGF-beta and the inhibitor of the antigen is a TGF beta inhibitor.

In one aspect, disclosed herein is method of treating a subject having a disease associated with the expression of BCMA, comprising:

administering a BCMA CAR-expressing cell therapy to the subject,

acquiring a value of the level or activity of an antigen in the subject, e.g., in a sample from the subject (e.g., a biopsy sample, e.g., a bone marrow biopsy sample), at at least one time point after the subject began receiving the BCMA CAR-expressing cell therapy,

responsive to an increase in the value relative to a reference value, wherein the reference value is:

(i) the level or activity of the antigen in the subject, e.g., in a sample from the subject (e.g., a biopsy sample, e.g., a bone marrow biopsy sample), prior to the at least one time point (e.g., the level or activity of the antigen in the subject before the subject began receiving the BCMA CAR-expressing cell therapy, or the level or activity of the antigen in the subject after the subject began receiving the BCMA CAR-expressing cell therapy but prior to the at least one time point);

(ii) the level or activity of the antigen in a different subject having the disease associated with the expression of BCMA; or

(iii) an average level or activity of the antigen in a population of subjects having the disease associated with the expression of BCMA,

administering an inhibitor of the antigen to the subject, wherein:

(1) the antigen is CD19 and the inhibitor of the antigen is a CD19 inhibitor, optionally wherein the CD19 inhibitor is:
(a) a CD19 CAR-expressing cell therapy, e.g., a CD19 CAR-expressing cell therapy disclosed herein, e.g., CTL119 or CTL019;
(b) a CAR-expressing cell therapy comprising a cell expressing a first CAR and a second CAR, wherein the first CAR is a BCMA CAR (e.g., a BCMA CAR disclosed herein) and the second CAR is a CD19 CAR (e.g., a CD19 CAR disclosed herein); or
(c) a CAR-expressing cell therapy comprising a cell expressing a multispecific CAR, e.g., a bispecific CAR, that binds to BCMA and CD19,

(2) the antigen is CD20 and the inhibitor of the antigen is a CD20 inhibitor, optionally wherein the CD20 inhibitor is:
(d) a CD20 CAR-expressing cell therapy, e.g., a CD20 CAR-expressing cell therapy disclosed herein;
(e) a CAR-expressing cell therapy comprising a cell expressing a first CAR and a second CAR, wherein the first CAR is a BCMA CAR (e.g., a BCMA CAR disclosed herein) and the second CAR is a CD20 CAR (e.g., a CD20 CAR disclosed herein);
(f) a CAR-expressing cell therapy comprising a cell expressing a multispecific CAR, e.g., a bispecific CAR, that binds to BCMA and CD20; or
(g) a multispecific antibody molecule, e.g., a bispecific antibody molecule, that binds to CD20 and CD3, e.g., THG338,

(3) the antigen is CD22 and the inhibitor of the antigen is a CD22 inhibitor, optionally wherein the CD22 inhibitor is:
(h) a CD22 CAR-expressing cell therapy, e.g., a CD22 CAR-expressing cell therapy disclosed herein;
(i) a CAR-expressing cell therapy comprising a cell expressing a first CAR and a second CAR, wherein the first CAR is a BCMA CAR (e.g., a BCMA CAR disclosed herein) and the second CAR is a CD22 CAR (e.g., a CD22 CAR disclosed herein); or
(j) a CAR-expressing cell therapy comprising a cell expressing a multispecific CAR, e.g., a bispecific CAR, that binds to BCMA and CD22,

(4) the antigen is PD1 or PD-L1 and the inhibitor of the antigen is an anti-PD1 antibody molecule or an anti-PD-L1 antibody molecule, optionally wherein the inhibitor of the antigen is:
(k) PDR001, Nivolumab, Pembrolizumab, Pidilizumab, MEDI0680, REGN2810, TSR-042, PF-06801591, or AMP-224; or
(l) FAZ053, Atezolizumab, Avelumab, Durvalumab, or BMS-936559,

(5) the antigen is IDO or TDO and the inhibitor of the antigen is an inhibitor of IDO and/or TDO, optionally wherein the inhibitor of IDO and/or TDO is:
(m) INCB24360, indoximod, NLG919, epacadostat, NLG919, or F001287; or
(n) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan, or

(6) the antigen is TGF-beta and the inhibitor of the antigen is a TGF beta inhibitor.

In certain embodiments of the foregoing aspects, the value of the level or activity of the antigen comprises the expression level of the antigen in the subject, e.g., in a sample from the subject (e.g., a biopsy sample, e.g., a bone marrow biopsy sample), as measured by an assay described herein, e.g., immunohistochemistry.

In some embodiments, the at least one time point is 5, 10, 15, 20, 25, 28, 30, 35, 40, 45, 50, 55, 60, 65, 70, 75, 80, or 90 days after the subject began receiving the BCMA CAR-expressing cell therapy.

In some embodiments, the subject experiences a decrease in BCMA expression after the subject began receiving the BCMA CAR-expressing cell therapy.

In certain embodiments of the foregoing aspects, the BCMA CAR-expressing cell therapy comprises a cell expressing a BCAM CAR. In some embodiments, the BCMA CAR comprises one or more of (e.g., all three of) heavy chain complementary determining region 1 (HCDR1), HCDR2, and HCDR3 listed in Table 3 or 5 and/or one or more of (e.g., all three of) light chain complementary determining region 1 (LCDR1), LCDR2, and LCDR3 listed in Table 4 or 5, or a sequence with 95-99% identify thereof. In some embodiments, the BCMA CAR comprises a heavy chain variable region (VH) listed in Table 2 or 5 and/or a light chain variable region (VL) listed in Table 2 or 5, or a sequence with 95-99% identify thereof. In some embodiments, the BCMA CAR comprises a BCMA scFv domain amino acid sequence listed in Table 2 or 5 (e.g., SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, and SEQ ID NO: 149), or a sequence with 95-99% identify thereof. In some embodiments, the BCMA CAR comprises a full-length BCMA CAR amino acid sequence listed in Table 2 or 5 (e.g., residues 22-483 of SEQ ID NO: 109, residues 22-490 of SEQ ID NO: 99, residues 22-488 of SEQ ID NO: 100, residues 22-487 of SEQ ID NO: 101, residues 22-493 of SEQ ID NO: 102, residues 22-490 of SEQ ID NO: 103, residues 22-491 of SEQ ID NO: 104, residues 22-482 of SEQ ID NO: 105, residues 22-483 of SEQ ID NO: 106, residues 22-485 of SEQ ID NO: 107, residues 22-483 of SEQ ID NO: 108, residues 22-490 of SEQ ID NO: 110, residues 22-483 of SEQ ID NO: 111, residues 22-484 of SEQ ID NO: 112, residues 22-485 of SEQ ID NO: 113, residues 22-487 of SEQ ID NO: 213, residues 23-489 of SEQ ID NO: 214, residues 22-490 of SEQ ID NO: 215, residues 22-484 of SEQ ID NO: 216, residues 22-485 of SEQ ID NO: 217, residues 22-489 of SEQ ID NO: 218, residues 22-497 of SEQ ID NO: 219, residues 22-492 of SEQ ID NO: 220, residues 22-490 of SEQ ID NO: 221, residues 22-485 of SEQ ID NO: 222, residues 22-492 of SEQ ID NO: 223, residues 22-492 of SEQ ID NO: 224, residues 22-483 of SEQ ID NO: 225, residues 22-490 of SEQ ID NO: 226, residues 22-485 of SEQ ID NO: 227, residues 22-486 of SEQ ID NO: 228, residues 22-492 of SEQ ID NO: 229, residues 22-488 of SEQ ID NO: 230, residues 22-488 of SEQ ID NO: 231, residues 22-495 of SEQ ID NO: 232, residues 22-490 of SEQ ID NO: 233), or a sequence with 95-99% identify thereof. In some embodiments, the BCMA CAR is encoded by a nucleic acid sequence listed in Table 2 or 5 (e.g., SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170), or a sequence with 95-99% identify thereof.

In certain embodiments of the foregoing aspects, the disease associated with the expression of BCMA is cancer, optionally wherein the cancer is a hematological cancer. In some embodiments, the disease associated with the expression of BCMA is an acute leukemia chosen from one or more of B-cell acute lymphoid leukemia (“BALL”), T-cell acute lymphoid leukemia (“TALL”), acute lymphoid leukemia (ALL); chronic myelogenous leukemia (CML), chronic lymphocytic leukemia (CLL); B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia; a prostate cancer (e.g., castrate-resistant or therapy-resistant prostate cancer, or metastatic prostate cancer), pancreatic cancer, lung cancer; or a plasma cell proliferative disorder (e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome)), or a combination thereof. In some embodiments, the disease associated with the expression of BCMA is ALL, CLL, DLBCL, or multiple myeloma. In some embodiments, the subject is a human patient.

The materials, methods, and examples are illustrative only and not intended to be limiting.

Headings, sub-headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are a pair of graphs showing the percentage of CD4+ or CD8+ T cells among CD3+ T cells (FIG. 1A) and the CD4:CD8 T cell ratio (FIG. 1B) in apheresis samples acquired from multiple myeloma patients who were later determined to be Responders (R, N_{R}=3) or Non-Responders (NR, N_{NR}=5) to treatment with an infusion of CART-BCMA. These data demonstrate that Responders had a higher percentage of CD4+ T cells and a lower percentage of CD8+ T cells (and thus a higher CD4:CD8 ratio) in their apheresis sample than Non-Responders did. A CD4:CD8 ratio greater than about 1.6 was found to be predictive of response to CART-BCMA.

FIGS. 2A, 2B, and 2C are graphs showing that the percentage of HLADR-CD95+CD27+CD8+ T cells (FIG. 2A), CD45RO−CD27+CD8+ T cells (FIG. 2B), or CCR7+CD45RO−CD27+CD8+ T cells (FIG. 2C) among CD8+ T cells is higher in apheresis samples acquired from multiple myeloma patients who were later determined to be Responders (R, N_{R}=3) to CART-BCMA as compared to Non-Responders (NR, N_{NR}=5). P-values are shown for each graph.

FIG. 3 is a series of images showing CD138+ cell localization as determined by immunohistochemistry (IHC) in bone marrow core biopsies acquired prior to administration (“Pre”), and on Day 28 and Day 90 (“3 month”) post-infusion of CART-BCMA from Patient 13, Patient 14, Patient 15, Patient 16, and Patient 17. Patient outcomes to treatment with CART-BCMA are provided in the Examples and are referred to herein as follows: Progressive disease (PD); Stable disease (SD); Minor response (MR); Partial regression (PR); and Very good partial regression (VGPR). Pretreatment, Day 28, and Day 90 samples acquired from Patient 13 had 1%, 0%, and 0% CD138+ MM cell infiltration, respectively. Pretreatment and Day 28 samples acquired from Patient 14 had 80% and 90% CD138+ MM cell infiltration, respectively. Pretreatment, Day 28, and Day 90 samples acquired from Patient 15 had 95%, 5%, and 10% CD138+ MM cell infiltration, respectively. Pretreatment, Day 28, and Day 90 samples acquired from Patient 16 had 50%, 5%, and 75% CD138+ MM cell infiltration, respectively. Pretreatment, Day 28, and Day 90 samples acquired from Patient 17 had 50%, 5%, and 75% CD138+ MM cell infiltration, respectively.

FIG. 4 is a series of images showing BCMA protein expression as determined by IHC in bone marrow core biopsies acquired prior to administration (“Pre”), and on Day 28 and Day 90 (“3 month”) post-infusion of CART-BCMA from Patient 13, Patient 14, Patient 15, Patient 16, and Patient 17.

FIG. 5 is a series of images showing a comparison between BCMA protein expression as determined by IHC to BCMA mRNA levels as determined by in situ hybridization (ISH) in bone marrow core biopsies acquired prior to administration of CART-BCMA from Patient 13, Patient 14, Patient 15, Patient 16, and Patient 17.

FIGS. 6A, 6B, and 6C are a series of images showing BCMA protein expression as determined by IHC, BCMA mRNA levels as determined by ISH, and CART-BCMA mRNA levels as determined by ISH in bone marrow core biopsies acquired from Patient 15 (FIG. 6A), Patient 16 (FIG. 6B), and Patient 17 (FIG. 6C), prior to administration (“Pre”), and on Day 28 and Day 90 (“3 month”) post-infusion of CART-BCMA.

FIGS. 7A, 7B, and 7C are a series of images showing IDO1, IFN-γ, and TGFβ mRNA levels as determined by ISH in bone marrow core biopsies acquired from Patient 15 (FIG. 7A), Patient 16 (FIG. 7B), and Patient 17 (FIG. 7C), prior to administration (“Pre”), and on Day 28 and Day 90 (“3 month”) post-infusion of CART-BCMA. FIGS. 7D and 7E are a series of images showing CAR, IFN-γ, and IDO1 mRNA levels as determined by ISH in biopsies acquired from Patient 19 (FIG. 7D) and Patient 20 (FIG. 7E), prior to administration (“Pre”), and on Day 10 and Day 28 post-infusion of CART-BCMA.

FIGS. 8A, 8B, and 8C are a series of images showing PD-L1, PD1, CD3, and FoxP3 protein expression as determined by IHC in bone marrow core biopsies acquired from Patient 15 (FIG. 8A), Patient 16 (FIG. 8B), and Patient 17 (FIG. 8C), prior to administration (“Pre”), and on Day 28 and Day 90 (“3 month”) post-infusion of CART-BCMA. FIGS. 8D and 8E are a series of images showing PD1, PD-L1, and FoxP3 protein expression as determined by IHC in biopsies acquired from Patient 19 (FIG. 8D) and Patient 20 (FIG. 8E), prior to administration (“Pre”), and on Day 10 and Day 28 post-infusion of CART-BCMA.

FIG. 9 is a series of images showing CD19 protein expression as determined by IHC in bone marrow core biopsies acquired prior to administration (“Pre”), and on Day 28 and Day 90 (“3 month”) post-infusion of CART-BCMA from Patient 13, Patient 14, Patient 15, Patient 16, and Patient 17.

FIG. 10 is a series of images showing CD20 protein expression as determined by IHC in bone marrow core biopsies acquired prior to administration (“Pre”), and on Day 28 and Day 90 (“3 month”) post-infusion of CART-BCMA from Patient 13, Patient 14, Patient 15, Patient 16, and Patient 17.

FIGS. 11A and 11B are a series of spectrally unmixed pseudo fluorescent microscopy images showing that BCMA positive cells and CD19 positive cells are separate populations in bone marrow core biopsies acquired from Patient 15 prior to administration (“pre”) and on Day 90 (“3M”) post-infusion of CAR-BCMA.

FIGS. 12A and 12B are a series of spectrally unmixed pseudo fluorescent microscopy images showing that CD19+CD34^{dim} cell population was present in the pretreatment bone marrow core biopsies acquired from Patient 15 and Patient 17, respectively.

FIG. 13 is a series of spectrally unmixed pseudo fluorescent microscopy images showing that the CD19 population was variably CD138+ and CD138− in the pretreatment bone marrow core biopsies acquired from Patient 15.

FIG. 14 is a graph comparing the level of tumor burden in a KMS11 tumor model following implant and administration of PBS, untransduced T cells (“UTD”), or T cells transduced with either a tool CAR (“J6MO”), BCMA-4, BCMA-9, BCMA-10 (“MCM998”), BCMA-13, or BCMA-15. BCMA-10 demonstrated the most potent anti-tumor activity.

FIG. 15 is a diagram showing the design of a clinical trial (NCT Number: NCT02546167; UPCC 14415) to assess the safety and feasibility of infusion of autologous T cells expressing CART-BCMA in adult patients with multiple myeloma.

FIG. 16A is a table showing MM patient disease characteristics. FIG. 16B is a table showing the presence of baseline lymphopenia due to disease and prior therapies in MM patients.

FIGS. 17A, 17B, and 17C are graphs showing patient response for Cohort 1, Cohort 2, and Cohort 3, respectively.

FIGS. 18A and 18B are a series of graphs showing expansion of CART-BCMA evaluated by flow cytometry in Cohort 1 patients and Cohort 2/3 patients, respectively.

FIGS. 19A and 19B are a series of graphs showing expansion of CART-BCMA evaluated by PCR in Cohort 1 patients and Cohort 2/3 patients, respectively. The plots show the number of detected CART genes per μg of DNA isolated from patient's blood (y-axis) at the respective day post CART infusion (x-axis).

FIGS. 20A and 20B are graphs showing that BCMA expansion may correlate with clinical outcomes.

FIGS. 21A, 21B, 21C, and 21D are graphs showing the fraction of CAR-positive (CAR+) CD4/CD8 cells at various time points post-infusion in Responders compared to Non-Responders.

FIG. 22 is a series of graphs showing the changes in level of cytokine expression at various time points post infusion of CART-BCMA. The y-axis in each graph shows fold change from Day 0. The x-axis in each graph shows days post-infusion of CART-BCMA.

FIGS. 23A and 23B are graphs showing the change in IL-6 expression at various time points post infusion of CART-BCMA. The y-axis in each graph shows fold change from Day 0. The x-axis in each graph shows days post-infusion of CART-BCMA.

FIGS. 24A and 24B are graphs showing the change in IFN-γ expression at various time points post infusion of CART-BCMA. The y-axis in each graph shows fold change from Day 0. The x-axis in each graph shows days post-infusion of CART-BCMA.

FIGS. 25A and 25B are graphs showing the serum level of BCMA in 14 normal donors (FIG. 25A) and 12 myeloma patients (FIG. 25B).

FIGS. 26A, 26B, 26C, and 26D are graphs showing serum BCMA level at various time points post infusion of CART-BCMA. The y-axis in FIGS. 26A and 26B shows peripheral blood (PB) serum BCMA levels. The y-axis in FIGS. 26C and 26D shows PB serum BCMA level fold change from baseline. The x-axis in each graph shows days post-infusion of CART-BCMA.

FIGS. 27A, 27B, and 27C are graphs showing data collected from three multiple myeloma patients who received CART-BCMA treatment. The y-axis on the left shows the percentage of CD4+ or CD8+ CART cells. The y-axis on the right shows the level of serum BCMA (ng/mL) or the number of CART copies (BBz) per μg DNA, as evaluated by qPCR.

FIGS. 28A and 28B are graphs showing CD4+ T cell subsets of normal donors (FIG. 28A) and multiple myeloma (MM) patients (FIG. 28B). FIGS. 28C and 28D are graphs showing CD8+ T cell subsets of normal donors (FIG. 28C) and MM patients (FIG. 28D). FIGS. 28E and 28F are graphs showing CD4+ and CD8+ T cell subsets, respectively, in apheresis samples acquired from MM patients (dots with slashes represent non-responders and white dots represent responders).

FIG. 29 is a series of graphs showing T cell differentiation in apheresis samples acquired from MM patients. The x-axis shows CD45RO expression and the y-axis shows CCR7 expression. Signal in the top left quadrant indicates naïve cell phenotype; signal in top right quadrant indicates central memory (T_{CM}) phenotype; signal in bottom right quadrant indicates effector memory (T_{EM}) phenotype; and signal in bottom left quadrant indicates T_{EMRA}. CR stands for complete response. PD stands for progressive disease. VGPR stands for very good partial response.

FIGS. 30A and 30B are a pair of graphs showing CD4+ and CD8+ T cell subsets in apheresis samples acquired from MM patients (dots with slashes represent non-responders and white dots represent responders).

FIG. 31 is a graph showing treatment schema.

FIGS. 32A, 32B, and 32C are a set of graphs showing clinical outcomes. FIG. 32A is a Swimmer's plot showing best response and progression-free survival (PFS) for each subject. Arrow indicates ongoing response. FIG. 32B is a pair of PET/CT scan images for subject 03 showing resolution of extramedullary disease and malignant pleural effusion post-treatment. FIG. 32C is a Kaplan-Meier plot showing overall survival for Cohort 1. MR=minimal response; MRD=minimal residual disease; PR=partial response; PD=progressive disease; sCR=stringent complete response; SD=stable disease.

FIGS. 33A, 33B, and 33C are a set of graphs showing CART-BCMA expansion and persistence. FIG. 33A is a set of graphs depicting CART-BCMA cell levels over time in peripheral blood for each subject, as measured by flow cytometry (% CAR+ within CD3+ T cells, ▴, left axis) and quantitative PCR for CAR sequence (▪, right axis). See FIG. 38 for representative flow cytometry plots. FIG. 33B is a graph showing that peak CART-BCMA levels by qPCR correlate with response: median 102507 vs. 4187 copies/μg DNA for ≥PR vs. <PR, respectively (p=0.016, Mann-Whitney). FIG. 33C is a graph showing that AUC-28 (area under the curve for CART-BCMA levels by qPCR during first 28 days after infusion) correlates with response: median 885181 vs. 26183 (copies)×(days)/μg DNA for ≥PR vs. <PR, respectively (p=0.016, Mann-Whitney).

FIG. 34 is a set of graphs showing soluble BCMA (sBCMA), BAFF, APRIL levels and B cell frequency after CART-BCMA infusions. Peripheral blood serum levels of sBCMA, BAFF, and APRIL (ng/ml, left axis) were measured by ELISA pre- and post-CART-BCMA infusions for each subject as indicated above. Subjects with deepest clinical responses (01 (sCR), 03 (VGPR), 15 (VGPR)) had greatest declines in sBCMA and reciprocal increases in BAFF and APRIL. Peripheral blood B cell frequency (% CD19+ of CD45+CD14− gate, right axis) was assessed by flow cytometry at indicated time points.

FIG. 35 is a set of histograms showing BCMA expression by flow cytometry on gated MM cells in marrow aspirates for each subject, before and after CART-BCMA infusions. Hatched histograms show BCMA; filled histograms show FMO (fluorescence minus one) control. Post-infusion time point is Day 28, unless specified. Percentage of cells expressing BCMA as well as mean BCMA fluorescence intensity (MFI) for each subject are listed in Table 37. Note decreased BCMA expression for subject 03 at relapse (D164). See FIG. 42 for representative gating.

FIGS. 36A, 36B, 36C, and 36D are a set of graphs showing predictors of in vivo CART-BCMA expansion. The ratio of CD4+ to CD8+ T cells (CD4/CD8 ratio) within the apheresis product immediately after collection (FIG. 36A) and within the seed culture at start of manufacturing (i.e. following elutriation step to reduce monocyte contamination) (FIG. 36B) was determined by flow cytometry. In vitro fold expansion (FIG. 36C) was calculated from total cell counts at start and end of manufacturing. The proportion of CD8+ T cells within the apheresis product with a CD45RO−CD27+ phenotype was assessed by flow cytometry (FIG. 36D). The CD4/CD8 ratio and frequency of CD45RO−CD27+CD8+ T cells pre-manufacturing, and degree of in vitro expansion were associated with peak in vivo CART-BCMA expansion post-infusion (Spearman correlation r and p-value shown).

FIG. 37 is a CONSORT diagram showing subject enrollment.

FIG. 38 is a set of graphs showing representative gating and staining for CART-BCMA cells. Staining is shown for peripheral blood from subject 01, day +7 after first CART-BCMA infusion. Cells are gated by forward and side scatter, then singlets, then CD45+CD14− leukocytes, then T cells (CD3+CD19−). CART-BCMA+ cells were identified using biotinylated recombinant human BCMA-Fc and streptavidin-PE. Negative control was an FMO (fluorescence minus one) tube (lacking biotinylated BCMA-Fc) with streptavidin-PE. The % of CD3+ T cells expressing CART-BCMA was calculated by subtracting CAR+ cells in FMO tube from CAR+ cells in tube with biotinylated BCMA-FC (i.e. in this example 34.7−0.9=33.8). Activation status of CART-BCMA+ cells was identified by staining for HLA-DR (bottom right panel). The % of CAR+ cells that were activated at each time point was calculated by dividing % HLA-DR+ by (% HLA-DR+ plus % HLA-DR-) (i.e. in this example 32.9/(32.9+1.5)=95.6%).

FIG. 39 is a set of graphs showing absolute number of CART-BCMA+ T cells for each subject. Absolute # of CD3+CAR+ cells per μl of blood was estimated from the absolute lymphocyte count (ALC, reported from the clinical complete blood count (CBC) differential) and the CART-BCMA flow cytometry results (FIG. 38), using the following formula: (ALC) (% CD45+CD14−)(% CD3+CD19−) (% CAR+)/10000. For example, for subject 01 at day +7, ALC was 0.08×10³ cells/μl. Estimated absolute # of circulating CAR+ T cells at this time point is (0.08)(48.3)(72.1)(33.8)/10000=0.019×10³ cells/μ1.

FIG. 40 is a set of graphs showing serum cytokine changes after CART-BCMA treatment. Levels of 30 peripheral blood cytokines were assessed at multiple time points by Luminex assay. Changes in selected cytokines over first 28 days are depicted. Subjects with deepest responses (01, 03, 15) had greatest fold-increase in cytokines, typically at or just before peak CART-BCMA expansion.

FIGS. 41A and 41B are a pair of graphs showing baseline soluble BCMA (sBCMA) levels, peak expansion, and response. Peripheral blood serum levels of sBCMA were measured by ELISA pre-treatment. FIG. 41A is a graph showing that baseline sBCMA levels did not correlate significantly with peak expansion of CART-BCMA by qPCR (Spearman correlation r=0.43, p=0.25). FIG. 41B is a graph showing that baseline levels of sBCMA were not significantly associated with response (p=0.56, Mann-Whitney test).

FIG. 42 is a set of graphs showing representative gating for myeloma cells and BCMA staining. Bone marrow aspirate cells were gated by forward and side scatter, then by singlets, then on CD3−CD14− cells. Myeloma cells were identified by gating first on CD38^{hi} cells, then by gating on clonal plasma cells using CD19, CD56, and kappa/lambda staining. In this example, myeloma cells are CD19-CD56+kappa+. The % BCMA+ was determined using an FMO tube lacking anti-BCMA antibody.

FIGS. 43A and 43B are a pair of graphs showing baseline BCMA expression on MM cells, peak expansion, and response. BCMA mean fluorescence intensity (MFI) on myeloma cells pre-treatment did not correlate with peak CART-BCMA expansion by qPCR (Spearman correlation r=0.45, p=0.27) (FIG. 43A), nor was it significantly associated with response (p=0.25, Mann-Whitney test) (FIG. 43B). One subject (07) did not have a pre-treatment sample available.

FIGS. 44A and 44B are a set of graphs showing BCMA expression on B cell malignancy cell lines. FIG. 44A is a set of histograms showing the surface expression of BCMA on each cell line. Hatched histograms indicate staining with PE-labeled anti-BCMA antibody and filled histograms show the respective isotype control staining. In FIG. 44B, expression was quantified and the antibody binding capacity (ABC) plotted for each cell line tested.

FIG. 45A is a graph showing % CD27+CD45RO−CD8+ cells in the post-induction cohort and the relapsed/refractory cohort. FIG. 45B is a graph showing CD4/CD8 ratio in the post-induction cohort and the relapsed/refractory cohort. FIG. 45C is a graph showing in vitro population doublings by Day 9 in the post-induction cohort and the relapsed/refractory cohort.

FIG. 46 is a graph showing treatment schema. BM asp/Bx=bone marrow aspirate and biopsy; Cytoxan=cyclophosphamide; D=day; Lenti=lentivirus; Wk=week.

FIGS. 47A-47C are a panel of swimmer's plots showing best response and progression-free survival (PFS) for each subject in Cohort 1 (1-5×10⁸ CART-BCMA cells alone) (FIG. 47A), Cohort 2 (Cyclophosphamide (Cy)+1-5×10⁷ CART-BCMA cells) (FIG. 47B), and Cohort 3 (Cy+1-5×10⁸ CART-BCMA cells) (FIG. 47C). Arrow indicates ongoing response. FIG. 47D is a graph showing overall survival (OS) based on cohort, Kaplan-Meier plot. MR=minimal response; MRD=minimal residual disease; PR=partial response; PD=progressive disease; sCR=stringent complete response; SD=stable disease.

FIGS. 48A-48D are graphs showing CART-BCMA expansion and persistence. FIGS. 48A-48C are graphs showing CART-BCMA cell levels over time in peripheral blood for each cohort, as measured by quantitative PCR for CAR sequence. FIG. 48D is a graph showing peak CART-BCMA levels by qPCR for each subject (except subj. 34, for whom peak data not available). Median peak CART-BCMA levels (grey bars) were not significantly different between cohorts (Mann-Whitney).

FIGS. 49A-49I are graphs showing serum cytokines associated with CRS severity and neurotoxicity. Serum cytokine concentrations in pg/ml through day 28 were measured by Luminex assay. FIGS. 49A-49E: The median peak fold increase over baseline for each cytokine was compared between subjects with no cytokine release syndrome (CRS), grade 1 CRS, or grade 2 CRS not receiving tocilizumab (CRS gr 0-2) and those with grade 3-4 CRS or grade 2 CRS receiving tocilizumab (CRS Gr 3-4 or Gr 2+toci). The cytokines most significantly associated with CRS severity were IL-6 (FIG. 49A), IFN-γ (FIG. 49B), IL-2Rα (FIG. 49C), MIP-1α (FIG. 49D), and IL-15 (FIG. 49E). FIGS. 49F-49i: Median peak fold increase over baseline for each cytokine was compared between subjects with no neurotoxicity (No Ntx) and those with any grade of neurotoxicity (Any Ntx). The cytokines most significantly associated with neurotoxicity were IL-6 (FIG. 49F), IFN-γ (FIG. 49G), IL-1RA (FIG. 49H), and MIP-la (FIG. 49I). Stars depict subjects with grade 3-4 neurotoxicity. Exact p-value by Mann-Whitney test is shown. Horizontal lines depict medians. IFN-γ=interferon gamma; IL-1RA=interleukin 1 receptor antagonist; IL-2Rα=interleukin 2 receptor alpha; IL-6=interleukin 6; IL-15=interleukin 15. MIP-1α=macrophage inflammatory protein 1 alpha.

FIGS. 50A-50D are graphs showing soluble BCMA (sBCMA), BAFF, and APRIL concentration, and BCMA expression on MM cells pre- and post-CART-BCMA infusions. FIG. 50A: Baseline peripheral blood serum concentration of sBCMA and APRIL for subjects (sub) were significantly increased and decreased, respectively, compared to a panel of healthy donors (HD, n=6) (p=0.017, and <0.001, respectively, Mann-Whitney). Baseline BAFF concentrations were not significantly different. Median concentrations are depicted. FIG. 50B: Serial sBCMA concentrations decline after CART-BCMA infusions more significantly in hematologic responders (PR/VGPR/CR/sCR) than non-responders (MR/SD/PD). Mean concentration (ng/ml)+SEM are depicted. *p<0.05 by unpaired t-test. FIG. 50C: Representative examples of BCMA expression on MM cells by flow cytometry. See FIG. 42 for gating strategy. FMO=fluorescence minus one. FIG. 50D: BCMA mean fluorescence intensity (MFI) on MM cells over time in 18 subjects with evaluable serial bone marrow aspirates. Median MFI was significantly different between pre-treatment (pre-tx) and day 28 (D28) for responders (4000 vs. 944, p=0.02, paired t-test) but not for non-responders (2704 vs. 2140, p=0.19). Median MFI was not significantly different between pre-tx and day 90 (D90) for responders (4000 vs. 2022, p=0.26). *Subj. 15 had no detectable MM cells at D28. #Subj. 03 had no detectable MM cells at D45 (D28 not done) and too few MM cells to characterize at D90. D164 marrow is depicted at D90 time-point.

FIGS. 51A-51I are graphs showing predictors of in vivo CART-BCMA expansion and response. Peak blood CART-BCMA expansion, as measured by qPCR (FIG. 51A), as well as total CART-BCMA expansion over first 28 days (calculated as area under the curve (AUC)) (FIG. 51B), were both associated with clinical response. Greater peak CART-BCMA expansion (FIG. 51C) and response (FIG. 51D) were also associated with more severe CRS, defined as grade ¾ or grade 2 requiring tocilizumab. A higher ratio of CD4+ to CD8+ T cells (CD4/CD8 ratio) within the leukopheresis product, as determined by flow cytometry, also correlated with both peak expansion (FIG. 51E) and response (FIG. 51F), while in vitro proliferation, measured as fold increase of seeded cells during manufacturing, correlated only with peak expansion (FIG. 51G), but not response (p=0.54, Mann-Whitney test, data not shown). FIGS. 51H-I: A higher proportion of CD8+ T cells within the leukopheresis product with a CD45RO−CD27+ phenotype was significantly associated with peak CART-BCMA expansion (FIG. 51H), and to a lesser degree, response (FIG. 51I). For FIGS. 51A, 51B, 51C, 51F, and 51I, analysis was by Mann-Whitney test; lines represent median values. For FIG. 51D, analysis was by Fisher's exact test. For FIGS. 51E, 51G, and 51H, analysis was by Spearman correlation.

FIG. 52 is a CONSORT diagram showing subject enrollment. ALC=absolute lymphocyte count.

FIGS. 53A-53D are graphs showing additional clinical outcomes for treated subjects. FIG. 53A: Duration of response (DOR) for all subjects with partial response (PR) or better. FIG. 53B: Overall survival (OS) for all subjects. FIG. 53C: Progression-free survival (PFS) by cohort. FIG. 53D: PFS for all subjects. Curves derived by Kaplan-Meier method.

FIGS. 54A-54C are graphs showing expansion of CART-BCMA cells for Cohort 1 (FIG. 54A), Cohort 2 (FIG. 54B) or Cohort 3 (FIG. 54C). The frequency of CAR+ T cells within all peripheral blood CD3+ T cells, as measured by flow cytometry, is depicted for each subject.

FIG. 55 is a panel of graphs showing serum cytokine changes after CART-BCMA treatment. Concentrations (pg/ml) of peripheral blood cytokines were assessed at multiple time-points by Luminex assay. The peak fold increase over baseline for the most frequently elevated cytokines over first 28 days post-infusion are shown, based on cohort.

FIGS. 56A-56L are graphs showing that peak CART-BCMA expansion is not associated with baseline clinical characteristics, baseline BCMA expression or sBCMA concentration. Peak CART-BCMA level (copies/μg genomic DNA) by qPCR was not significantly associated with age at enrollment (above or below median) (FIG. 56A); years from diagnosis (above or below median) (FIG. 56B); presence of dell7p by FISH or TP53 mutation by sequencing (FIG. 56C); number (#) of therapeutic lines (above or below median) (FIG. 56D); being penta-refractory to 2 proteasome inhibitors (PIs), 2 immunomodulatory drugs (IMiDs) and daratumumab (dara) (FIG. 56E); receiving therapy just prior to leukapheresis that contained an IMiD (FIG. 56F), a PI (FIG. 56G), dara (FIG. 56H), or cyclophosphamide (Cytoxan) (FIG. 56I); percentage of pre-treatment bone marrow plasma cells (% BM PC) (FIG. 56J); baseline BCMA mean fluorescence intensity (MFI) on BM PC (FIG. 56K); or baseline serum soluble BCMA (sBCMA) concentration (FIG. 56L). For FIGS. 56A-56I, analysis by Mann-Whitney test; line represents median value. For FIGS. 56J-56L, analysis by Spearman correlation.

FIGS. 57A-57L are graphs showing that response is not associated with baseline clinical characteristics, baseline BCMA expression or sBCMA concentration. Clinical response (≥partial response (PR)) was not significantly associated with age at enrollment (FIG. 57A); years from diagnosis (FIG. 57B); presence of dell7p by FISH or TP53 mutation by sequencing (FIG. 57C); number (#) of therapeutic lines (FIG. 57D); being penta-refractory to 2 proteasome inhibitors (PIs), 2 immunomodulatory drugs (IMiDs) and daratumumab (dara) (FIG. 57E); receiving a regimen just prior to leukapheresis that contained an IMiD, a PI, dara, or cyclophosphamide (Cytoxan) (FIGS. 57F-57I); percentage of pre-treatment bone marrow plasma cells (% BM PC) (FIG. 57J); baseline BCMA mean fluorescence intensity (MFI) on BM PC (FIG. 57K); or baseline serum soluble BCMA (sBCMA) concentration (FIG. 57L). For FIGS. 57C, 57E-57I, analysis by Fisher Exact test. For FIGS. 57A, 57B, 57D, 57J-57L, analysis by Mann-Whitney test; line represents median value.

### DESCRIPTION

### Definitions

Unless defined otherwise, all technical and scientific terms used herein have the same meaning as commonly understood by one of ordinary skill in the art to which the invention pertains.

As used herein, the term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells, and plasma cells. Its ligand is called B-cell activator of the TNF family (BAFF) and a proliferation inducing ligand (APRIL). BCMA is involved in mediating the survival of plasma cells for mataining long-term humoral immunity. The gene for BCMA is encoded on chromosome 16 producing a primary mRNA transcript of 994 nucleotides in length (NCBI accession NM_001192.2) that encodes a protein of 184 amino acids (NP_001183.2). A second antisense transcript derived from the BCMA locus has been described, which may play a role in regulating BCMA expression. (Laabi Y. et al., Nucleic Acids Res., 1994, 22:1147-1154). Additional transcript variants have been described with unknown significance (Smirnova A S et al. Mol Immunol., 2008, 45(4):1179-1183. A second isoform, also known as TV4, has been identified (Uniprot identifier Q02223-2). As used herein, “BCMA” includes proteins comprising mutations, e.g., point mutations, fragments, insertions, deletions and splice variants of full length wild-type BCMA.

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukemia, chronic lymphocyte leukemia and non-Hodgkin lymphoma. Other cells with express CD19 are provided below in the definition of “disease associated with expression of CD19.” It is also an early marker of B cell progenitors. See, e.g., Nicholson et al. Mol. Immun 34 (16-17): 1157-1165 (1997). In one aspect the antigen-binding portion of the CART recognizes and binds an antigen within the extracellular domain of the CD19 protein. In one aspect, the CD19 protein is expressed on a cancer cell.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In one aspect, the stimulatory molecule of the CAR is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4 1BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMACAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide brudge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The portion of the CAR composition of the invention comprising an antibody or antibody fragment thereof may exist in a variety of forms, for example, where the antigen binding domain is expressed as part of a polypeptide chain including, for example, a single domain antibody fragment (sdAb), a single chain antibody (scFv), or e.g., a humanized antibody (Harlow et at, 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises an scFv.

As used herein, the term “binding domain” or “antibody molecule” (also referred to herein as “anti-target (e.g., BCMA) binding domain”) refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival. The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present invention and a combination partner, are both administered to a patient simultaneously in the form of a single entity or dosage. The term “non-fixed combination” means that the active ingredients, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Preferred cancers treated by the methods described herein include multiple myeloma, Hodgkin's lymphoma or non-Hodgkin's lymphoma.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of BCMA” includes, but is not limited to, a disease associated with a cell which expresses BCMA (e.g., wild-type or mutant BCMA) or condition associated with a cell which expresses BCMA (e.g., wild-type or mutant BCMA) including, e.g., proliferative diseases such as a cancer or malignancy or a precancerous condition such as a myelodysplasia, a myelodysplastic syndrome or a preleukemia; or a noncancer related indication associated with a cell which expresses BCMA (e.g., wild-type or mutant BCMA). For the avoidance of doubt, a disease associated with expression of BCMA may include a condition associated with a cell which does not presently express BCMA, e.g., because BCMA expression has been downregulated, e.g., due to treatment with a molecule targeting BCMA, e.g., a BCMA inhibitor described herein, but which at one time expressed BCMA. In one aspect, a cancer associated with expression of BCMA (e.g., wild-type or mutant BCMA) is a hematological cancer. In one aspect, the hematological cancer is a leukemia or a lymphoma. In one aspect, a cancer associated with expression of BCMA (e.g., wild-type or mutant BCMA) is a malignancy of differentiated plasma B cells. In one aspect, a cancer associated with expression of BCMA (e.g., wild-type or mutant BCMA) includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of BMCA (e.g., wild-type or mutant BCMA) comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. In some embodiments, the cancer is multiple myeloma, Hodgkin's lymphoma, non-Hodgkin's lymphoma, or glioblastoma. In embodiments, a disease associated with expression of BCMA includes a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). Further diseases associated with expression of BCMA (e.g., wild-type or mutant BCMA) expression include, but not limited to, e.g., atypical and/or non-classical cancers, malignancies, precancerous conditions or proliferative diseases associated with expression of BCMA (e.g., wild-type or mutant BCMA), e.g., a cancer described herein, e.g., a prostate cancer (e.g., castrate-resistant or therapy-resistant prostate cancer, or metastatic prostate cancer), pancreatic cancer, or lung cancer.

Non-cancer related conditions that are associated with BCMA (e.g., wild-type or mutant BCMA) include viral infections; e.g., HIV, fungal infections, e.g., C. neoformans; autoimmune disease; e.g. rheumatoid arthritis, system lupus erythematosus (SLE or lupus), pemphigus vulgaris, and Sjogren's syndrome; inflammatory bowel disease, ulcerative colitis; transplant-related allospecific immunity disorders related to mucosal immunity; and unwanted immune responses towards biologics (e.g., Factor VIII) where humoral immunity is important. In embodiments, a non-cancer related indication associated with expression of BCMA includes but is not limited to, e.g., autoimmune disease, (e.g., lupus), inflammatory disorders (allergy and asthma) and transplantation. In some embodiments, the tumor antigen-expressing cell expresses, or at any time expressed, mRNA encoding the tumor antigen. In an embodiment, the tumor antigen-expressing cell produces the tumor antigen protein (e.g., wild-type or mutant), and the tumor antigen protein may be present at normal levels or reduced levels. In an embodiment, the tumor antigen-expressing cell produced detectable levels of a tumor antigen protein at one point, and subsequently produced substantially no detectable tumor antigen protein.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation,” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FccRI and CD66d, DAP10 and DAP12. In a specific CAR of the invention, the intracellular signaling domain in any one or more CARS of the invention comprises an intracellular signaling sequence, e.g., a primary signaling sequence of CD3-zeta. The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FccRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In one embodiment, the cytoplasmic domain of zeta comprises residues 52 through 164 of GenBank Acc. No. BAG36664.1 or a variant thereof (e.g., a molecule having mutations, e.g., point mutations, fragments, insertions, or deletions). In one embodiment, the “zeta stimulatory domain” or a “CD3-zeta stimulatory domain” is the sequence provided as SEQ ID NO: 1027 or 1030 or a variant thereof (e.g., a molecule having mutations, e.g., point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (e.g., a molecule having mutations, e.g., point mutations, fragments, insertions, or deletions). In one embodiment, the “4-1BB costimulatory domain” is the sequence provided as SEQ ID NO: 1022 or a variant thereof (e.g., a molecule having mutations, e.g., point mutations, fragments, insertions, or deletions).

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions, e.g., conservative substitutions), alleles, orthologs, SNPs, and complementary sequences as well as the sequence explicitly indicated. Specifically, degenerate codon substitutions, e.g., conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n (SEQ ID NO: 1038), where n is a positive integer equal to or greater than 1. For example, n=1, n=2, n=3. n=4, n=5 and n=6, n=7, n=8, n=9 and n=10 In one embodiment, the flexible polypeptide linkers include, but are not limited to, (Gly4 Ser)4 (SEQ ID NO: 1039) or (Gly4 Ser)3 (SEQ ID NO: 1040). In another embodiment, the linkers include multiple repeats of (Gly2Ser), (GlySer) or (Gly3Ser) (SEQ ID NO: 1041). Also included within the scope of the invention are linkers described in WO2012/138475, incorporated herein by reference.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000 (SEQ ID NO: 1043), preferably greater than 64, more preferably greater than 100, most preferably greater than 300 or 400 (SEQ ID NO: 2024). poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (e.g., castrate-resistant or therapy-resistant prostate cancer, or metastatic prostate cancer), ovarian cancer, pancreatic cancer, and the like, or a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In an embodiment the RCARX cell is a T cell, and is referred to as a RCART cell. In an embodiment the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g, RAD001.

The term “bioequivalent” refers to an amount of an agent other than the reference compound (e.g., RAD001), required to produce an effect equivalent to the effect produced by the reference dose or reference amount of the reference compound (e.g., RAD001). In an embodiment the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In an embodiment, the effect is alteration of the ratio of PD-1 positive/PD-1 negative T cells, as measured by cell sorting. In an embodiment a bioequivalent amount or dose of an mTOR inhibitor is the amount or dose that achieves the same level of P70 S6 kinase inhibition as does the reference dose or reference amount of a reference compound. In an embodiment, a bioequivalent amount or dose of an mTOR inhibitor is the amount or dose that achieves the same level of alteration in the ratio of PD-1 positive/PD-1 negative T cells as does the reference dose or reference amount of a reference compound.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive immune effector cells, e.g., T cells or NK cells, and/or an increase in the number of PD-1 negative immune effector cells, e.g., T cells or NK cells, or an increase in the ratio of PD-1 negative immune effector cells (e.g., T cells or NK cells)/PD-1 positive immune effector cells (e.g., T cells or NK cells).

In an embodiment, the low, immune enhancing, dose of mTOR inhibitor results in an increase in the number of naive T cells. In an embodiment, the low, immune enhancing, dose of mTOR inhibitor results in one or more of the following:

an increase in the expression of one or more of the following markers: CD62Lhigh, CD127high, CD27+, and BCL2, e.g., on memory T cells, e.g., memory T cell precursors;

a decrease in the expression of KLRG1, e.g., on memory T cells, e.g., memory T cell precursors; and

an increase in the number of memory T cell precursors, e.g., cells with any one or combination of the following characteristics: increased CD62Lhigh, increased CD127high, increased CD27+, decreased KLRG1, and increased BCL2;

wherein any of the changes described above occurs, e.g., at least transiently, e.g., as compared to a non-treated subject.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or a “relapse” as used herein refers to the reappearance of a disease (e.g., cancer) or the signs and symptoms of a disease such as cancer after a period of improvement or responsiveness, e.g., after prior treatment of a therapy, e.g., cancer therapy. For example, the period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%.

In one aspect, a “responder” of a therapy can be a subject having complete response, very good partial response, or partial response after receiving the therapy. In one aspect, a “non-responder” of a therapy can be a subject having minor response, stable disease, or progressive disease after receiving the therapy. In some embodiments, the subject has multiple myeloma and the response of the subject to a multiple myeloma therapy is determined based on IMWG 2016 criteria, e.g., as disclosed in Kumar, et al., Lancet Oncol. 17, e328-346 (2016), hereby incorporated herein by reference in its entirety, e.g., as described in Table 7.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The term “alkyl,” as used herein, refers to a monovalent saturated, straight- or branched-chain hydrocarbon such as a straight or branched group of 1-12, 1-10, or 1-6 carbon atoms, referred to herein as C₁-C₁₂alkyl, C₁-C₁₀ alkyl, and C₁-C₆ alkyl, respectively. Examples of alkyl groups include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The terms “alkenyl” and “alkynyl” as used herein refer to unsaturated aliphatic groups analogous in length and possible substitution to the alkyls described above, but that contain at least one double or triple bond, respectively. Exemplary alkenyl groups include, but are not limited to, —CH═CH₂ and —CH₂CH═CH₂.

The term “aryl” as used herein refers to a monocyclic, bicyclic or polycyclic hydrocarbon ring system, wherein at least one ring is aromatic. Representative aryl groups include fully aromatic ring systems, such as phenyl (e.g., (C₆) aryl), naphthyl (e.g., (C₁₀) aryl), and anthracenyl (e.g., (C₁₄) aryl), and ring systems where an aromatic carbon ring is fused to one or more non-aromatic carbon rings, such as indanyl, phthalimidyl, naphthimidyl, or tetrahydronaphthyl, and the like.

The term “carbocyclyl” as used herein refers to monocyclic, or fused, spiro-fused, and/or bridged bicyclic or polycyclic hydrocarbon ring system containing 3-18 carbon atoms, wherein each ring is either completely saturated or contains one or more units of unsaturation, but where no ring is aromatic. Representative carbocyclyl groups include cycloalkyl groups (e.g., cyclopentyl, cyclobutyl, cyclopentyl, cyclohexyl and the like), and cycloalkenyl groups (e.g., cyclopentenyl, cyclohexenyl, cyclopentadienyl, and the like).

The term “carbonyl” as used herein refers to —C═O.

The term “cyano” as used herein refers to —CN.

The terms “halo” or “halogen” as used herein refer to fluorine (fluoro, —F), chlorine (chloro, —Cl), bromine (bromo, —Br), or iodine (iodo, —I).

The term “heteroalkyl” as used herein refers to a monovalent saturated straight or branched alkyl chain wherein at least one carbon atom in the chain is replaced with a heteroatom, such as O, S, or N, provided that upon substitution, the chain comprises at least one carbon atom. In some embodiments, a heteroalkyl group may comprise, e.g., 1-12, 1-10, or 1-6 carbon atoms, referred to herein as C₁-C₁₂ heteroalkyl, heteroalkyl, and C₁-C₆heteroalkyl. In certain instances, a heteroalkyl group comprises 1, 2, 3, or 4 independently selected heteroatoms in place of 1, 2, 3, or 4 individual carbon atoms in the alkyl chain. Representative heteroalkyl groups include —CH₂NHC(O)CH₃, —CH₂CH₂OCH₃, —CH₂CH₂NHCH₃, —CH₂CH₂N(CH₃)CH₃, and the like.

The term “heteroaryl” as used herein refers to a monocyclic, bicyclic or polycyclic ring system wherein at least one ring is both aromatic and comprises a heteroatom; and wherein no other rings are heterocyclyl (as defined below). Representative heteroaryl groups include ring systems where (i) each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3(4H)-one, thiazolo-[4,5-c]-pyridinyl, 4,5,6,7-tetrahydrothieno[2,3-c]pyridinyl, 5,6-dihydro-4H-thieno[2,3-c]pyrrolyl, 4,5,6,7,8-tetrahydroquinolinyl and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl. In certain embodiments, the heteroaryl is a monocyclic or bicyclic ring, wherein each of said rings contains 5 or 6 ring atoms where 1, 2, 3, or 4 of said ring atoms are a heteroatom independently selected from N, O, and S.

The term “heterocyclyl” as used herein refers to a monocyclic, or fused, spiro-fused, and/or bridged bicyclic and polycyclic ring systems where at least one ring is saturated or partially unsaturated (but not aromatic) and comprises a heteroatom. A heterocyclyl can be attached to its pendant group at any heteroatom or carbon atom that results in a stable structure and any of the ring atoms can be optionally substituted. Representative heterocyclyls include ring systems in which (i) every ring is non-aromatic and at least one ring comprises a heteroatom, e.g., tetrahydrofuranyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl; and (iii) at least one ring is non-aromatic and comprises a heteroatom and at least one other ring is aromatic and comprises a heteroatom, e.g., 3,4-dihydro-1H-pyrano[4,3-c]pyridinyl, and 1,2,3,4-tetrahydro-2,6-naphthyridinyl. In certain embodiments, the heterocyclyl is a monocyclic or bicyclic ring, wherein each of said rings contains 3-7 ring atoms where 1, 2, 3, or 4 of said ring atoms are a heteroatom independently selected from N, O, and S.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The term “oxo” as used herein refers to ═O.

The term “thiocarbonyl” as used herein refers to C═S.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺ (C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I), Formula (I-a), and/or Formula (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H2O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

It is to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “tautomers” refer to compounds that are interchangeable forms of a particular compound structure, and that vary in the displacement of hydrogen atoms and electrons. Thus, two structures may be in equilibrium through the movement of 7t electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base.

Tautomeric forms may be relevant to the attainment of the optimal chemical reactivity and biological activity of a compound of interest.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972).

Various aspects of the compositions and methods herein are described in further detail below. Additional definitions are set out throughout the specification.

### DETAILED DESCRIPTION

The present invention provides, at least in part, a method of treating a subject having a disease associated with BCMA expression, comprising administering to the subject an effective amount of a cell (e.g., a population of cells) that expresses a CAR molecule that binds BCMA (a “BCMA CAR-expressing cell”). In some embodiments, the disease associated with expression of BCMA is a hematologic cancer, e.g., ALL, CLL, DLBCL, or multiple myeloma. In some embodiments, the BCMA CAR-expressing cell therapy is administered based on the acquisition of a level of a biomarker from a patient sample. In some embodiments, the BCMA CAR-expressing cell therapy is administered to the subject in combination with a second therapy. In some embodiments, the BCMA CAR-expressing cell therapy and the second therapy are administered simultaneously or sequentially.

### Chimeric Antigen Receptor (CAR)

In one aspect, disclosed herein are methods using a cell (e.g., a population of cells) that expresses a CAR molecule. In one aspect, an exemplary CAR construct comprises an optional leader sequence (e.g., a leader sequence described herein), an antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

Sequences of non-limiting examples of various components that can be part of a CAR molecule described herein, are listed in Table 1, where “aa” stands for amino acids, and “na” stands for nucleic acids that encode the corresponding peptide.

TABLE 2
Sequences of various components of CAR (aa-amino acid sequence, na-nucleic acid
sequence).

| SEQ ID | | |
|---|---|---|
| NO: | description | Sequence |
| 1007 | EF-1 | CGTGAGGCTCCGGTGCCCGTCAGTGGGCAGAGCGCACATCGC |
| | promoter | CCACAGTCCCCGAGAAGTTGGGGGGAGGGGTCGGCAATTGAA |
| | | CCGGTGCCTAGAGAAGGTGGCGCGGGGTAAACTGGGAAAGT |
| | | GATGTCGTGTACTGGCTCCGCCTTTTTCCCGAGGGTGGGGGAG |
| | | AACCGTATATAAGTGCAGTAGTCGCCGTGAACGTTCTTTTTCG |
| | | CAACGGGTTTGCCGCCAGAACACAGGTAAGTGCCGTGTGTGG |
| | | TTCCCGCGGGCCTGGCCTCTTTACGGGTTATGGCCCTTGCGTG |
| | | CCTTGAATTACTTCCACCTGGCTGCAGTACGTGATTCTTGATC |
| | | CCGAGCTTCGGGTTGGAAGTGGGTGGGAGAGTTCGAGGCCTT |
| | | GCGCTTAAGGAGCCCCTTCGCCTCGTGCTTGAGTTGAGGCCTG |
| | | GCCTGGGCGCTGGGGCCGCCGCGTGCGAATCTGGTGGCACCT |
| | | TCGCGCCTGTCTCGCTGCTTTCGATAAGTCTCTAGCCATTTAA |
| | | AATTTTTGATGACCTGCTGCGACGCTTTTTTTCTGGCAAGATA |
| | | GTCTTGTAAATGCGGGCCAAGATCTGCACACTGGTATTTCGGT |
| | | TTTTGGGGCCGCGGGCGGCGACGGGGCCCGTGCGTCCCAGCG |
| | | CACATGTTCGGCGAGGCGGGGCCTGCGAGCGCGGCCACCGAG |
| | | AATCGGACGGGGGTAGTCTCAAGCTGGCCGGCCTGCTCTGGT |
| | | GCCTGGCCTCGCGCCGCCGTGTATCGCCCCGCCCTGGGCGGC |
| | | AAGGCTGGCCCGGTCGGCACCAGTTGCGTGAGCGGAAAGATG |
| | | GCCGCTTCCCGGCCCTGCTGCAGGGAGCTCAAAATGGAGGAC |
| | | GCGGCGCTCGGGAGAGCGGGCGGGTGAGTCACCCACACAAA |
| | | GGAAAAGGGCCTTTCCGTCCTCAGCCGTCGCTTCATGTGACTC |
| | | CACGGAGTACCGGGCGCCGTCCAGGCACCTCGATTAGTTCTC |
| | | GAGCTTTTGGAGTACGTCGTCTTTAGGTTGGGGGGAGGGGTTT |
| | | TATGCGATGGAGTTTCCCCACACTGAGTGGGTGGAGACTGAA |
| | | GTTAGGCCAGCTTGGCACTTGATGTAATTCTCCTTGGAATTTG |
| | | CCCTTTTTGAGTTTGGATCTTGGTTCATTCTCAAGCCTCAGAC |
| | | AGTGGTTCAAAGTTTTTTTCTTCCATTTCAGGTGTCGTGA |
| 1008 | Leader (aa) | MALPVTALLLPLALLLHAARP |
| 1009 | Leader (na) | ATGGCCCTGCCTGTGACAGCCCTGCTGCTGCCTCTGGCTCTGC |
| | | TGCTGCATGCCGCTAGACCC |
| 1010 | Leader (na) | ATGGCCCTCCCTGTCACCGCCCTGCTGCTTCCGCTGGCTCTTC |
| | | TGCTCCACGCCGCTCGGCCC |
| 1011 | CD8 hinge | TTTPAPRPPTPAPTIASQPLSLRPEACRPAAGGAVHTRGLDFACD |
| | (aa) | |
| 1012 | CD8 hinge | ACCACGACGCCAGCGCCGCGACCACCAACACCGGCGCCCACC |
| | (na) | ATCGCGTCGCAGCCCCTGTCCCTGCGCCCAGAGGCGTGCCGG |
| | | CCAGCGGCGGGGGGCGCAGTGCACACGAGGGGGCTGGACTT |
| | | CGCCTGTGAT |
| 1013 | Ig4 hinge | ESKYGPPCPPCPAPEFLGGPSVFLFPPKPKDTLMISRTPEVTCVVV |
| | (aa) | DVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLGKM |
| 1014 | Ig4 hinge | GAGAGCAAGTACGGCCCTCCCTGCCCCCCTTGCCCTGCCCCCG |
| | (na) | AGTTCCTGGGCGGACCCAGCGTGTTCCTGTTCCCCCCCAAGCC |
| | | CAAGGACACCCTGATGATCAGCCGGACCCCCGAGGTGACCTG |
| | | TGTGGTGGTGGACGTGTCCCAGGAGGACCCCGAGGTCCAGTT |
| | | CAACTGGTACGTGGACGGCGTGGAGGTGCACAACGCCAAGAC |
| | | CAAGCCCCGGGAGGAGCAGTTCAATAGCACCTACCGGGTGGT |
| | | GTCCGTGCTGACCGTGCTGCACCAGGACTGGCTGAACGGCAA |
| | | GGAATACAAGTGTAAGGTGTCCAACAAGGGCCTGCCCAGCAG |
| | | CATCGAGAAAACCATCAGCAAGGCCAAGGGCCAGCCTCGGG |
| | | AGCCCCAGGTGTACACCCTGCCCCCTAGCCAAGAGGAGATGA |
| | | CCAAGAACCAGGTGTCCCTGACCTGCCTGGTGAAGGGCTTCT |
| | | ACCCCAGCGACATCGCCGTGGAGTGGGAGAGCAACGGCCAG |
| | | CCCGAGAACAACTACAAGACCACCCCCCCTGTGCTGGACAGC |
| | | GACGGCAGCTTCTTCCTGTACAGCCGGCTGACCGTGGACAAG |
| | | AGCCGGTGGCAGGAGGGCAACGTCTTTAGCTGCTCCGTGATG |
| | | CACGAGGCCCTGCACAACCACTACACCCAGAAGAGCCTGAGC |
| | | CTGTCCCTGGGCAAGATG |
| 1015 | IgD hinge | RWPESPKAQASSVPTAQPQAEGSLAKATTAPATTRNTGRGGEEK |
| | (aa) | KKEKEKEEQEERETKTPECPSHTQPLGVYLLTPAVQDLWLRDKA |
| | | TFTCFVVGSDLKDAHLTWEVAGKVPTGGVEEGLLERHSNGSQS |
| | | QHSRLTLPRSLWNAGTSVTCTLNHPSLPPQRLMALREPAAQAPV |
| | | KLSLNLLASSDPPEAASWLLCEVSGFSPPNILLMWLEDQREVNTS |
| | | GFAPARPPPQPGSTTFWAWSVLRVPAPPSPQPATYTCVVSHEDS |
| | | RTLLNASRSLEVSYVTDH |
| 1016 | IgD hinge | AGGTGGCCCGAAAGTCCCAAGGCCCAGGCATCTAGTGTTCCT |
| | (na) | ACTGCACAGCCCCAGGCAGAAGGCAGCCTAGCCAAAGCTACT |
| | | ACTGCACCTGCCACTACGCGCAATACTGGCCGTGGCGGGGAG |
| | | GAGAAGAAAAAGGAGAAAGAGAAAGAAGAACAGGAAGAGA |
| | | GGGAGACCAAGACCCCTGAATGTCCATCCCATACCCAGCCGC |
| | | TGGGCGTCTATCTCTTGACTCCCGCAGTACAGGACTTGTGGCT |
| | | TAGAGATAAGGCCACCTTTACATGTTTCGTCGTGGGCTCTGAC |
| | | CTGAAGGATGCCCATTTGACTTGGGAGGTTGCCGGAAAGGTA |
| | | CCCACAGGGGGGGTTGAGGAAGGGTTGCTGGAGCGCCATTCC |
| | | AATGGCTCTCAGAGCCAGCACTCAAGACTCACCCTTCCGAGA |
| | | TCCCTGTGGAACGCCGGGACCTCTGTCACATGTACTCTAAATC |
| | | ATCCTAGCCTGCCCCCACAGCGTCTGATGGCCCTTAGAGAGC |
| | | CAGCCGCCCAGGCACCAGTTAAGCTTAGCCTGAATCTGCTCG |
| | | CCAGTAGTGATCCCCCAGAGGCCGCCAGCTGGCTCTTATGCG |
| | | AAGTGTCCGGCTTTAGCCCGCCCAACATCTTGCTCATGTGGCT |
| | | GGAGGACCAGCGAGAAGTGAACACCAGCGGCTTCGCTCCAGC |
| | | CCGGCCCCCACCCCAGCCGGGTTCTACCACATTCTGGGCCTGG |
| | | AGTGTCTTAAGGGTCCCAGCACCACCTAGCCCCCAGCCAGCC |
| | | ACATACACCTGTGTTGTGTCCCATGAAGATAGCAGGACCCTG |
| | | CTAAATGCTTCTAGGAGTCTGGAGGTTTCCTACGTGACTGACC |
| | | ATT |
| 1017 | GS | GGGGSGGGGS |
| | hinge/linker | |
| | (aa) | |
| 1018 | GS | GGTGGCGGAGGTTCTGGAGGTGGAGGTTCC |
| | hinge/linker | |
| | (na) | |
| 1019 | CD8 | IYIWAPLAGTCGVLLLSLVITLYC |
| | transmembrane | |
| | (TM) (aa) | |
| 1020 | CD8 | ATCTACATCTGGGCGCCCTTGGCCGGGACTTGTGGGGTCCTTC |
| | transmembrane | TCCTGTCACTGGTTATCACCCTTTACTGC |
| | (TM) (na) | |
| 1021 | CD8 TM (na) | ATCTACATTTGGGCCCCTCTGGCTGGTACTTGCGGGGTCCTGC |
| | | TGCTTTCACTCGTGATCACTCTTTACTGT |
| 1022 | 4-1BB | KRGRKKLLYIFKQPFMRPVQTTQEEDGCSCRFPEEEEGGCEL |
| | intracellular | |
| | domain (aa) | |
| 1023 | 4-1BB | AAACGGGGCAGAAAGAAACTCCTGTATATATTCAAACAACCA |
| | intracellular | TTTATGAGACCAGTACAAACTACTCAAGAGGAAGATGGCTGT |
| | domain (na) | AGCTGCCGATTTCCAGAAGAAGAAGAAGGAGGATGTGAACT |
| | | G |
| 1024 | 4-1BB | AAGCGCGGTCGGAAGAAGCTGCTGTACATCTTTAAGCAACCC |
| | intracellular | TTCATGAGGCCTGTGCAGACTACTCAAGAGGAGGACGGCTGT |
| | domain (na) | TCATGCCGGTTCCCAGAGGAGGAGGAAGGCGGCTGCGAACTG |
| 1025 | CD27 (aa) | QRRKYRSNKGESPVEPAEPCRYSCPREEEGSTIPIQEDYRKPEPAC |
| | | SP |
| 1026 | CD27 (na) | AGGAGTAAGAGGAGCAGGCTCCTGCACAGTGACTACATGAAC |
| | | ATGACTCCCCGCCGCCCCGGGCCCACCCGCAAGCATTACCAG |
| | | CCCTATGCCCCACCACGCGACTTCGCAGCCTATCGCTCC |
| 1027 | CD3-zeta | RVKFSRSADAPAYKQGQNQLYNELNLGRREEYDVLDKRRGRDP |
| | (aa) | EMGGKPRRKNPQEGLYNELQKDKMAEAYSEIGMKGERRRGKG |
| | | HDGLYQGLSTATKDTYDALHMQALPPR |
| 1028 | CD3-zeta | AGAGTGAAGTTCAGCAGGAGCGCAGACGCCCCCGCGTACAA |
| | (na) | GCAGGGCCAGAACCAGCTCTATAACGAGCTCAATCTAGGACG |
| | | AAGAGAGGAGTACGATGTTTTGGACAAGAGACGTGGCCGGG |
| | | ACCCTGAGATGGGGGGAAAGCCGAGAAGGAAGAACCCTCAG |
| | | GAAGGCCTGTACAATGAACTGCAGAAAGATAAGATGGCGGA |
| | | GGCCTACAGTGAGATTGGGATGAAAGGCGAGCGCCGGAGGG |
| | | GCAAGGGGCACGATGGCCTTTACCAGGGTCTCAGTACAGCCA |
| | | CCAAGGACACCTACGACGCCCTTCACATGCAGGCCCTGCCCC |
| | | CTCGC |
| 1029 | CD3-zeta | CGCGTGAAATTCAGCCGCAGCGCAGATGCTCCAGCCTACAAG |
| | (na) | CAGGGGCAGAACCAGCTCTACAACGAACTCAATCTTGGTCGG |
| | | AGAGAGGAGTACGACGTGCTGGACAAGCGGAGAGGACGGGA |
| | | CCCAGAAATGGGCGGGAAGCCGCGCAGAAAGAATCCCCAAG |
| | | AGGGCCTGTACAACGAGCTCCAAAAGGATAAGATGGCAGAA |
| | | GCCTATAGCGAGATTGGTATGAAAGGGGAACGCAGAAGAGG |
| | | CAAAGGCCACGACGGACTGTACCAGGGACTCAGCACCGCCAC |
| | | CAAGGACACCTATGACGCTCTTCACATGCAGGCCCTGCCGCC |
| | | TCGG |
| 1030 | CD3-zeta | RVKFSRSADAPAYQQGQNQLYNELNLGRREEYDVLDKRRGRDP |
| | (aa) | EMGGKPRRKNPQEGLYNELQKDKMAEAYSEIGMKGERRRGKG |
| | | HDGLYQGLSTATKDTYDALHMQALPPR |
| 1031 | CD3-zeta | AGAGTGAAGTTCAGCAGGAGCGCAGACGCCCCCGCGTACCAG |
| | (na) | CAGGGCCAGAACCAGCTCTATAACGAGCTCAATCTAGGACGA |
| | | AGAGAGGAGTACGATGTTTTGGACAAGAGACGTGGCCGGGA |
| | | CCCTGAGATGGGGGGAAAGCCGAGAAGGAAGAACCCTCAGG |
| | | AAGGCCTGTACAATGAACTGCAGAAAGATAAGATGGCGGAG |
| | | GCCTACAGTGAGATTGGGATGAAAGGCGAGCGCCGGAGGGG |
| | | CAAGGGGCACGATGGCCTTTACCAGGGTCTCAGTACAGCCAC |
| | | CAAGGACACCTACGACGCCCTTCACATGCAGGCCCTGCCCCC |
| | | TCGC |
| 1032 | linker | GGGGS |
| 1033 | linker | GGTGGCGGAGGTTCTGGAGGTGGAGGTTCC |
| 1034 | PD-1 | Pgwfldspdrpwnpptfspallvvtegdnatftcsfsntsesfvlnwyrmspsnqtdklaafpedrs |
| | extracellular | qpgqdcrfrvtqlpngrdfhmsvvrarrndsgtylcgaislapkaqikeslraelrvterraevptahp |
| | domain (aa) | spsprpagqfqtlv |
| 1035 | PD-1 | Cccggatggtttctggactctccggatcgcccgtggaatcccccaaccttctcaccggcactcttggtt |
| | extracellular | gtgactgagggcgataatgcgaccttcacgtgctcgttctccaacacctccgaatcattcgtgctgaact |
| | domain (na) | ggtaccgcatgagcccgtcaaaccagaccgacaagctcgccgcgtttccggaagatcggtcgcaac |
| | | cgggacaggattgtcggttccgcgtgactcaactgccgaatggcagagacttccacatgagcgtggtc |
| | | cgcgctaggcgaaacgactccgggacctacctgtgcggagccatctcgctggcgcctaaggcccaa |
| | | atcaaagagagcttgagggccgaactgagagtgaccgagcgcagagctgaggtgccaactgcacat |
| | | ccatccccatcgcctcggcctgcggggcagtttcagaccctggtc |
| 1036 | PD-1 CAR | Malpvtalllplalllhaarppgwfldspdrpwnpptfspallvvtegdnatftcsfsntsesfvlnwy |
| | (aa) with | rmspsnqtdklaafpedrsqpgqdcrfrvtqlpngrdfhmsvvrarrndsgtylcgaislapkaqik |
| | signal | eslraelrvterraevptahpspsprpagqfqtlvtapaprpptpaptiasqplslrpeacrpaaggav |
| | | htrgldfacdiyiwaplagtcgvlllslvitlyckrgrkkllyifkqpfmrpvqttqeedgcscrfpeee |
| | | eggcelrvkfsrsadapaykqgqnqlynelnlgrreeydvldkrrgrdpemggkprrknpqegly |
| | | nelqkdkmaeayseigmkgerrrgkghdglyqglstatkdtydalhmqalppr |
| 1037 | PD-1 CAR | Atggccctccctgtcactgccctgcttctccccctcgcactcctgctccacgccgctagaccacccgg |
| | (na) | atggtttctggactctccggatcgcccgtggaatcccccaaccttctcaccggcactcttggttgtgactg |
| | | agggcgataatgcgaccttcacgtgctcgttctccaacacctccgaatcattcgtgctgaactggtaccg |
| | | catgagcccgtcaaaccagaccgacaagctcgccgcgtttccggaagatcggtcgcaaccgggaca |
| | | ggattgtcggttccgcgtgactcaactgccgaatggcagagacttccacatgagcgtggtccgcgcta |
| | | ggcgaaacgactccgggacctacctgtgcggagccatctcgctggcgcctaaggcccaaatcaaag |
| | | agagcttgagggccgaactgagagtgaccgagcgcagagctgaggtgccaactgcacatccatccc |
| | | catcgcctcggcctgcggggcagtttcagaccctggtcacgaccactccggcgccgcgcccaccga |
| | | ctccggccccaactatcgcgagccagcccctgtcgctgaggccggaagcatgccgccctgccgccg |
| | | gaggtgctgtgcatacccggggattggacttcgcatgcgacatctacatttgggctcctctcgccggaa |
| | | cttgtggcgtgctccttctgtccctggtcatcaccctgtactgcaagcggggtcggaaaaagcttctgta |
| | | cattttcaagcagcccttcatgaggcccgtgcaaaccacccaggaggaggacggttgctcctgccggt |
| | | tccccgaagaggaagaaggaggttgcgagctgcgcgtgaagttctcccggagcgccgacgccccc |
| | | gcctataagcagggccagaaccagctgtacaacgaactgaacctgggacggcgggaagagtacgat |
| | | gtgctggacaagcggcgcggccgggaccccgaaatgggcgggaagcctagaagaaagaaccctc |
| | | aggaaggcctgtataacgagctgcagaaggacaagatggccgaggcctactccgaaattgggatga |
| | | agggagagcggcggaggggaaaggggcacgacggcctgtaccaaggactgtccaccgccaccaa |
| | | ggacacatacgatgccctgcacatgcaggcccttccccctcgc |
| 1038 | linker | (Gly-Gly-Gly-Ser)n, where n = 1-10 |
| 1039 | linker | (Gly4 Ser)4 |
| 1040 | linker | (Gly4 Ser)3 |
| 1041 | linker | (Gly3Ser) |
| 1042 | linker | ASGGGGSGGRASGGGGS |
| 1043 | polyA | [a]₅₀₋₅₀₀₀ |
| 1044 | PD1 CAR | Pgwfldspdrpwnpptfspallvvtegdnatftcsfsntsesfvlnwyrmspsnqtdklaafpedrs |
| | (aa) | gpgqdcrfrvtqlpngrdfhmsvvrarrndsgtylcgaislapkaqikeslraelrvterraevptahp |
| | | spsprpagqfqtlvtttpaprpptpaptiasqplslrpeacrpaaggavhtrgldfacdiyiwaplagt |
| | | cgvlllslvitlyckrgrkkllyifkqpfmrpvqttqeedgcscrfpeeeeggcelrvkfsrsadapay |
| | | kqgqnqlynelnlgrreeydvldlargrdpemggkprrknpqeglynelqkdkmaeayseigm |
| | | kgerrrgkghdglyqglstatkdtydalhmqalppr |
| 1045 | ICOS | TKKKYSSSVHDPNGEYMFMRAVNTAKKSRLTDVTL |
| | intracellular | |
| | domain (aa) | |
| 1046 | ICOS | ACAAAAAAGAAGTATTCATCCAGTGTGCACGACCCTAACGGT |
| | intracellular | GAATACATGTTCATGAGAGCAGTGAACACAGCCAAAAAATCC |
| | domain (na) | AGACTCACAGATGTGACCCTA |
| 1047 | ICOS TM | TTTPAPRPPTPAPTIASQPLSLRPEACRPAAGGAVHTRGLDFACD |
| | domain (aa) | FWLPIGCAAFVVVCILGCILICWL |
| 1048 | ICOS TM | ACCACGACGCCAGCGCCGCGACCACCAACACCGGCGCCCACC |
| | domain (na) | ATCGCGTCGCAGCCCCTGTCCCTGCGCCCAGAGGCGTGCCGG |
| | | CCAGCGGCGGGGGGCGCAGTGCACACGAGGGGGCTGGACTT |
| | | CGCCTGTGATTTCTGGTTACCCATAGGATGTGCAGCCTTTGTT |
| | | GTAGTCTGCATTTTGGGATGCATACTTATTTGTTGGCTT |
| 1049 | CD28 | RSKRSRLLHSDYMNMTPRRPGPTRKHYQPYAPPRDFAAYRS |
| | intracellular | |
| | domain (aa) | |
| 1050 | CD28 | AGGAGTAAGAGGAGCAGGCTCCTGCACAGTGACTACATGAAC |
| | intracellular | ATGACTCCCCGCCGCCCCGGGCCCACCCGCAAGCATTACCAG |
| | domain (na) | CCCTATGCCCCACCACGCGACTTCGCAGCCTATCGCTCC |

### CAR Antigen Binding Domain

In one aspect, the portion of the CAR comprising the antigen binding domain comprises an antigen binding domain that targets a tumor antigen, e.g., a tumor antigen described herein. In some embodiments, the antigen binding domain binds to: CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2; Mesothelin; Interleukin 11 receptor alpha (IL-11Ra); prostate stem cell antigen (PSCA); Protease Serine 21; vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGSS); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like, Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

The antigen binding domain can be any domain that binds to an antigen, including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CAR Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CART.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD27, CD3 epsilon, CD45, CD4, CDS, CD8, CD9, CD16, CD22, CD33, CD37, CD64, CD80, CD86, CD134, CD137, CD154. In some embodiments, a transmembrane domain may include at least the transmembrane region(s) of, e.g., KIR2DS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of SEQ ID NO: 1011. In one aspect, the transmembrane domain comprises (e.g., consists of) a transmembrane domain of SEQ ID NO: 1019.

In one aspect, the hinge or spacer comprises an IgG4 hinge. For example, in one embodiment, the hinge or spacer comprises a hinge of the amino acid sequence of SEQ ID NO: 1013. In some embodiments, the hinge or spacer comprises a hinge encoded by a nucleotide sequence of SEQ ID NO: 1014.

In one aspect, the hinge or spacer comprises an IgD hinge. For example, in one embodiment, the hinge or spacer comprises a hinge of the amino acid sequence of SEQ ID NO: 1015. In some embodiments, the hinge or spacer comprises a hinge encoded by a nucleotide sequence of SEQ ID NO: 1016.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of SEQ ID NO: 1017. In some embodiments, the linker is encoded by a nucleotide sequence of SEQ ID NO: 1018.

In one aspect, the hinge or spacer comprises a KIR2DS2 hinge.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in a CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FccRI, DAP10, DAP12, and CD66d. In one embodiment, a CAR of the invention comprises an intracellular signaling domain, e.g., a primary signaling domain of CD3-zeta, e.g., a CD3-zeta sequence described herein.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

### Costimulatory Signaling Domain

The intracellular signalling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp30, NKp44, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, NKG2D, NKG2C and PAG/Cbp.

The intracellular signaling sequences within the cytoplasmic portion of the CAR may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 1022. In one aspect, the signaling domain of CD3-zeta is a signaling domain of SEQ ID NO: 1027.

In one aspect, the intracellular signaling domain is designed to comprise the signaling domain of CD3-zeta and the signaling domain of CD27. In one aspect, the signaling domain of CD27 comprises an amino acid sequence of SEQ ID NO: 1025. In one aspect, the signalling domain of CD27 is encoded by a nucleic acid sequence of SEQ ID NO: 1026.

In one aspect, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein, e.g., CD19, CD33, CLL-1, CD34, FLT3, or folate receptor beta). In one embodiment, the second CAR includes an antigen binding domain to a target expressed the same cancer cell type as the cancer associated antigen. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, ICOS, CD27 or OX-40, onto the first CAR, and the primary signaling domain, e.g., CD3 zeta, on the second CAR can limit the CAR activity to cells where both targets are expressed. In one embodiment, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In another aspect, the disclosure features a population of CAR-expressing cells, e.g., CART cells. In some embodiments, the population of CAR-expressing cells comprises a mixture of cells expressing different CARs. For example, in one embodiment, the population of CART cells can include a first cell expressing a CAR having an antigen binding domain to a cancer associated antigen described herein, and a second cell expressing a CAR having a different antigen binding domain, e.g., an antigen binding domain to a different a cancer associated antigen described herein, e.g., an antigen binding domain to a cancer associated antigen described herein that differs from the cancer associate antigen bound by the antigen binding domain of the CAR expressed by the first cell. As another example, the population of CAR-expressing cells can include a first cell expressing a CAR that includes an antigen binding domain to a cancer associated antigen described herein, and a second cell expressing a CAR that includes an antigen binding domain to a target other than a cancer associate antigen as described herein. In one embodiment, the population of CAR-expressing cells includes, e.g., a first cell expressing a CAR that includes a primary intracellular signaling domain, and a second cell expressing a CAR that includes a secondary signaling domain.

In another aspect, the disclosure features a population of cells wherein at least one cell in the population expresses a CAR having an antigen binding domain to a cancer associated antigen described herein, and a second cell expressing another agent, e.g., an agent which enhances the activity of a CAR-expressing cell. For example, in one embodiment, the agent can be an agent which inhibits an inhibitory molecule. Inhibitory molecules, e.g., PD-1, can, in some embodiments, decrease the ability of a CAR-expressing cell to mount an immune effector response. Examples of inhibitory molecules include PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGFbeta). In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4 and TGF beta, or a fragment of any of these, and a second polypeptide which is an intracellular signaling domain described herein (e.g., comprising a costimulatory domain (e.g., 41BB, CD27, OX40 or CD28, e.g., as described herein) and/or a primary signaling domain (e.g., a CD3 zeta signaling domain described herein). In one embodiment, the agent comprises a first polypeptide of PD-1 or a fragment thereof, and a second polypeptide of an intracellular signaling domain described herein (e.g., a CD28 signaling domain described herein and/or a CD3 zeta signaling domain described herein).

### BCMA CAR

In one aspect, the CAR disclosed herein binds to BCMA. Exemplary BCMA CARs can include sequences disclosed in Table 1 or 16 of WO2016/014565, incorporated herein by reference. The BCMA CAR construct can include an optional leader sequence; an optional hinge domain, e.g., a CD8 hinge domain; a transmembrane domain, e.g., a CD8 transmembrane domain; an intracellular domain, e.g., a 4-1BB intracellular domain; and a functional signaling domain, e.g., a CD3 zeta domain. In certain embodiments, the domains are contiguous and in the same reading frame to form a single fusion protein. In other embodiments, the domain are in separate polypeptides, e.g., as in an RCAR molecule as described herein.

The sequences of exemplary BCMA CAR molecules or fragments thereof are disclosed in Tables 2-5. In certain embodiments, the full length BCMA CAR molecule includes one or more CDRs, VH, VL, scFv, or full-length sequences of, BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1, as disclosed in Tables 2-5, or a sequence substantially (e.g., 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, U.S. Pat. Nos. 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA CAR constructs are generated using the VH and VL sequences from PCT Publication WO2012/0163805 (the contents of which are hereby incorporated by reference in its entirety).

TABLE 2
Amino Acid and Nucleic Acid Sequences of exemplary anti-BCMA scFv domains and
BCMA CAR molecules. The amino acid sequences variable heavy chain and
variable light chain sequences for each scFv is also provided.

| | | |
|---|---|---|
| | SEQ | |
| Name/ | ID | |
| Description | NO: | Sequence |

139109

| | | |
|---|---|---|
| 139109-aa | 49 | EVQLVESGGGLVQPGGSLRLSCAVSGFALSNHGMSWVRRAPGKGLEWVS |
| ScFv domain | | GIVYSGSTYYAASVKGRFTISRDNSRNTLYLQMNSLRPEDTAIYYCSAH |
| | | GGESDVWGQGTTVTVSSASGGGGSGGRASGGGGSDIQLTQSPSSLSASV |
| | | GDRVTITCRASQSISSYLNWYQQKPGKAPKLLIYAASSLQSGVPSRFSG |
| | | SGSGTDFTLTISSLQPEDFATYYCQQSYSTPYTFGQGTKVEIK |
| 139109-nt | 64 | GAAGTGCAATTGGTGGAATCAGGGGGAGGACTTGTGCAGCCTGGAGGAT |
| ScFv domain | | CGCTGAGACTGTCATGTGCCGTGTCCGGCTTTGCCCTGTCCAACCACGG |
| | | GATGTCCTGGGTCCGCCGCGCGCCTGGAAAGGGCCTCGAATGGGTGTCG |
| | | GGTATTGTGTACAGCGGTAGCACCTACTATGCCGCATCCGTGAAGGGGA |
| | | GATTCACCATCAGCCGGGACAACTCCAGGAACACTCTGTACCTCCAAAT |
| | | GAATTCGCTGAGGCCAGAGGACACTGCCATCTACTACTGCTCCGCGCAT |
| | | GGCGGAGAGTCCGACGTCTGGGGACAGGGGACCACCGTGACCGTGTCTA |
| | | GCGCGTCCGGCGGAGGCGGCAGCGGGGGTCGGGCATCAGGGGGCGGCGG |
| | | ATCGGACATCCAGCTCACCCAGTCCCCGAGCTCGCTGTCCGCCTCCGTG |
| | | GGAGATCGGGTCACCATCACGTGCCGCGCCAGCCAGTCGATTTCCTCCT |
| | | ACCTGAACTGGTACCAACAGAAGCCCGGAAAAGCCCCGAAGCTTCTCAT |
| | | CTACGCCGCCTCGAGCCTGCAGTCAGGAGTGCCCTCACGGTTCTCCGGC |
| | | TCCGGTTCCGGTACTGATTTCACCCTGACCATTTCCTCCCTGCAACCGG |
| | | AGGACTTCGCTACTTACTACTGCCAGCAGTCGTACTCCACCCCCTACAC |
| | | TTTCGGACAAGGCACCAAGGTCGAAATCAAG |
| 139109-aa | 79 | EVQLVESGGGLVQPGGSLRLSCAVSGFALSNHGMSWVRRAPGKGLEWVS |
| VH | | GIVYSGSTYYAASVKGRFTISRDNSRNTLYLQMNSLRPEDTAIYYCSAH |
| | | GGESDVWGQGTTVTVSS |
| 139109-aa | 94 | DIQLTQSPSSLSASVGDRVTITCRASQSISSYLNWYQQKPGKAPKLLIY |
| VL | | AASSLQSGVPSRFSGSGSGTDFTLTISSLQPEDFATYYCQQSYSTPYTF |
| | | GQGTKVEIK |
| 139109-aa | 109 | MALPVTALLLPLALLLHAARPEVQLVESGGGLVQPGGSLRLSCAVSGFA |
| Full CAR | | LSNHGMSWVRRAPGKGLEWVSGIVYSGSTYYAASVKGRFTISRDNSRNT |
| | | LYLQMNSLRPEDTAIYYCSAHGGESDVWGQGTTVTVSSASGGGGSGGRA |
| | | SGGGGSDIQLTQSPSSLSASVGDRVTITCRASQSISSYLNWYQQKPGKA |
| | | PKLLIYAASSLQSGVPSRFSGSGSGTDFTLTISSLQPEDFATYYCQQSY |
| | | STPYTFGQGTKVEIKTTTPAPRPPTPAPTIASQPLSLRPEACRPAAGGA |
| | | VHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 139109-nt | 124 | ATGGCCCTCCCTGTCACCGCCCTGCTGCTTCCGCTGGCTCTTCTGCTCC |
| Full CAR | | ACGCCGCTCGGCCCGAAGTGCAATTGGTGGAATCAGGGGGAGGACTTGT |
| | | GCAGCCTGGAGGATCGCTGAGACTGTCATGTGCCGTGTCCGGCTTTGCC |
| | | CTGTCCAACCACGGGATGTCCTGGGTCCGCCGCGCGCCTGGAAAGGGCC |
| | | TCGAATGGGTGTCGGGTATTGTGTACAGCGGTAGCACCTACTATGCCGC |
| | | ATCCGTGAAGGGGAGATTCACCATCAGCCGGGACAACTCCAGGAACACT |
| | | CTGTACCTCCAAATGAATTCGCTGAGGCCAGAGGACACTGCCATCTACT |
| | | ACTGCTCCGCGCATGGCGGAGAGTCCGACGTCTGGGGACAGGGGACCAC |
| | | CGTGACCGTGTCTAGCGCGTCCGGCGGAGGCGGCAGCGGGGGTCGGGCA |
| | | TCAGGGGGCGGCGGATCGGACATCCAGCTCACCCAGTCCCCGAGCTCGC |
| | | TGTCCGCCTCCGTGGGAGATCGGGTCACCATCACGTGCCGCGCCAGCCA |
| | | GTCGATTTCCTCCTACCTGAACTGGTACCAACAGAAGCCCGGAAAAGCC |
| | | CCGAAGCTTCTCATCTACGCCGCCTCGAGCCTGCAGTCAGGAGTGCCCT |
| | | CACGGTTCTCCGGCTCCGGTTCCGGTACTGATTTCACCCTGACCATTTC |
| | | CTCCCTGCAACCGGAGGACTTCGCTACTTACTACTGCCAGCAGTCGTAC |
| | | TCCACCCCCTACACTTTCGGACAAGGCACCAAGGTCGAAATCAAGACCA |
| | | CTACCCCAGCACCGAGGCCACCCACCCCGGCTCCTACCATCGCCTCCCA |
| | | GCCTCTGTCCCTGCGTCCGGAGGCATGTAGACCCGCAGCTGGTGGGGCC |
| | | GTGCATACCCGGGGTCTTGACTTCGCCTGCGATATCTACATTTGGGCCC |
| | | CTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCC |
| | | GGTTCCCAGAGGAGGAGGAAGGCGGCTGCGAACTGCGCGTGAAATTCAG |
| | | CCGCAGCGCAGATGCTCCAGCCTACAAGCAGGGGCAGAACCAGCTCTAC |
| | | AACGAACTCAATCTTGGTCGGAGAGAGGAGTACGACGTGCTGGACAAGC |
| | | GGAGAGGACGGGACCCAGAAATGGGCGGGAAGCCGCGCAGAAAGAATCC |
| | | CCAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCACG |
| | | ACGGACTGTACCAGGGACTCAGCACCGCCACCAAGGACACCTATGACGC |
| | | TCTTCACATGCAGGCCCTGCCGCCTCGG |
| Full CAR | 392 | EVQLVESGGGLVQPGGSLRLSCAVSGFALSNHGMSWVRRAPGKGLEWVS |
| without | | GIVYSGSTYYAASVKGRFTISRDNSRNTLYLQMNSLRPEDTAIYYCSAH |
| leader | | GGESDVWGQGTTVTVSSASGGGGSGGRASGGGGSDIQLTQSPSSLSASV |
| sequence | | GDRVTITCRASQSISSYLNWYQQKPGKAPKLLIYAASSLQSGVPSRFSG |
| | | SGSGTDFTLTISSLQPEDFATYYCQQSYSTPYTFGQGTKVEIKTTTPAP |
| | | RPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEE |
| | | EEGGCELRVKFSRSADAPAYKQGQNQLYNELNLGRREEYDVLDKRRGRD |
| | | PEMGGKPRRKNPQEGLYNELQKDKMAEAYSEIGMKGERRRGKGHDGLYQ |
| | | GLSTATKDTYDALHMQALPPR |
| Full CAR | 393 | EVQLVESGGGLVQPGGSLRLSCAVSGFALSNHGMSWVRRAPGKGLEWVS |
| without | | GIVYSGSTYYAASVKGRFTISRDNSRNTLYLQMNSLRPEDTAIYYCSAH |
| linker, | | GGESDVWGQGTTVTVSSDIQLTQSPSSLSASVGDRVTITCRASQSISSY |
| without | | LNWYQQKPGKAPKLLIYAASSLQSGVPSRFSGSGSGTDFTLTISSLQPE |
| leader | | DFATYYCQQSYSTPYTFGQGTKVEIKTTTPAPRPPTPAPTIASQPLSLR |
| sequence | | PEACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQA |
| | | LPPR |

139103

| | | |
|---|---|---|
| 139103-aa | 39 | QVQLVESGGGLVQPGRSLRLSCAASGFTFSNYAMSWVRQAPGKGLGWVS |
| ScFv domain | | GISRSGENTYYADSVKGRFTISRDNSKNTLYLQMNSLRDEDTAVYYCAR |
| | | SPAHYYGGMDVWGQGTTVTVSSASGGGGSGGRASGGGGSDIVLTQSPGT |
| | | LSLSPGERATLSCRASQSISSSFLAWYQQKPGQAPRLLIYGASRRATGI |
| | | PDRFSGSGSGTDFTLTISRLEPEDSAVYYCQQYHSSPSWTFGQGTKLEI |
| | | K |
| 139103-nt | 54 | CAAGTGCAACTCGTGGAATCTGGTGGAGGACTCGTGCAACCCGGAAGAT |
| ScFv domain | | CGCTTAGACTGTCGTGTGCCGCCAGCGGGTTCACTTTCTCGAACTACGC |
| | | GATGTCCTGGGTCCGCCAGGCACCCGGAAAGGGACTCGGTTGGGTGTCC |
| | | GGCATTTCCCGGTCCGGCGAAAATACCTACTACGCCGACTCCGTGAAGG |
| | | GCCGCTTCACCATCTCAAGGGACAACAGCAAAAACACCCTGTACTTGCA |
| | | AATGAACTCCCTGCGGGATGAAGATACAGCCGTGTACTATTGCGCCCGG |
| | | TCGCCTGCCCATTACTACGGCGGAATGGACGTCTGGGGACAGGGAACCA |
| | | CTGTGACTGTCAGCAGCGCGTCGGGTGGCGGCGGCTCAGGGGGTCGGGC |
| | | CTCCGGGGGGGGAGGGTCCGACATCGTGCTGACCCAGTCCCCGGGAACC |
| | | CTGAGCCTGAGCCCGGGAGAGCGCGCGACCCTGTCATGCCGGGCATCCC |
| | | AGAGCATTAGCTCCTCCTTTCTCGCCTGGTATCAGCAGAAGCCCGGACA |
| | | GGCCCCGAGGCTGCTGATCTACGGCGCTAGCAGAAGGGCTACCGGAATC |
| | | CCAGACCGGTTCTCCGGCTCCGGTTCCGGGACCGATTTCACCCTTACTA |
| | | TCTCGCGCCTGGAACCTGAGGACTCCGCCGTCTACTACTGCCAGCAGTA |
| | | CCACTCATCCCCGTCGTGGACGTTCGGACAGGGCACCAAGCTGGAGATT |
| | | AAG |
| 139103-aa | 69 | QVQLVESGGGLVQPGRSLRLSCAASGFTFSNYAMSWVRQAPGKGLGWVS |
| VH | | GISRSGENTYYADSVKGRFTISRDNSKNTLYLQMNSLRDEDTAVYYCAR |
| | | SPAHYYGGMDVWGQGTTVTVSS |
| 139103-aa | 84 | DIVLTQSPGTLSLSPGERATLSCRASQSISSSFLAWYQQKPGQAPRLLI |
| VL | | YGASRRATGIPDRFSGSGSGTDFTLTISRLEPEDSAVYYCQQYHSSPSW |
| | | TFGQGTKLEIK |
| 139103-aa | 99 | MALPVTALLLPLALLLHAARPQVQLVESGGGLVQPGRSLRLSCAASGFT |
| Full CAR | | FSNYAMSWVRQAPGKGLGWVSGISRSGENTYYADSVKGRFTISRDNSKN |
| | | TLYLQMNSLRDEDTAVYYCARSPAHYYGGMDVWGQGTTVTVSSASGGGG |
| | | SGGRASGGGGSDIVLTQSPGTLSLSPGERATLSCRASQSISSSFLAWYQ |
| | | QKPGQAPRLLIYGASRRATGIPDRFSGSGSGTDFTLTISRLEPEDSAVY |
| | | YCQQYHSSPSWTFGQGTKLEIKTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 139103-nt | 114 | ATGGCCCTCCCTGTCACCGCCCTGCTGCTTCCGCTGGCTCTTCTGCTCC |
| Full CAR | | ACGCCGCTCGGCCCCAAGTGCAACTCGTGGAATCTGGTGGAGGACTCGT |
| | | GCAACCCGGAAGATCGCTTAGACTGTCGTGTGCCGCCAGCGGGTTCACT |
| | | TTCTCGAACTACGCGATGTCCTGGGTCCGCCAGGCACCCGGAAAGGGAC |
| | | TCGGTTGGGTGTCCGGCATTTCCCGGTCCGGCGAAAATACCTACTACGC |
| | | CGACTCCGTGAAGGGCCGCTTCACCATCTCAAGGGACAACAGCAAAAAC |
| | | ACCCTGTACTTGCAAATGAACTCCCTGCGGGATGAAGATACAGCCGTGT |
| | | ACTATTGCGCCCGGTCGCCTGCCCATTACTACGGCGGAATGGACGTCTG |
| | | GGGACAGGGAACCACTGTGACTGTCAGCAGCGCGTCGGGTGGCGGCGGC |
| | | TCAGGGGGTCGGGCCTCCGGGGGGGGAGGGTCCGACATCGTGCTGACCC |
| | | AGTCCCCGGGAACCCTGAGCCTGAGCCCGGGAGAGCGCGCGACCCTGTC |
| | | ATGCCGGGCATCCCAGAGCATTAGCTCCTCCTTTCTCGCCTGGTATCAG |
| | | CAGAAGCCCGGACAGGCCCCGAGGCTGCTGATCTACGGCGCTAGCAGAA |
| | | GGGCTACCGGAATCCCAGACCGGTTCTCCGGCTCCGGTTCCGGGACCGA |
| | | TTTCACCCTTACTATCTCGCGCCTGGAACCTGAGGACTCCGCCGTCTAC |
| | | TACTGCCAGCAGTACCACTCATCCCCGTCGTGGACGTTCGGACAGGGCA |
| | | CCAAGCTGGAGATTAAGACCACTACCCCAGCACCGAGGCCACCCACCCC |
| | | GGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAG |
| | | AGGAGGACGGCTGTTCATGCCGGTTCCCAGAGGAGGAGGAAGGCGGCTG |
| | | CGAACTGCGCGTGAAATTCAGCCGCAGCGCAGATGCTCCAGCCTACAAG |
| | | CAGGGGCAGAACCAGCTCTACAACGAACTCAATCTTGGTCGGAGAGAGG |
| | | AGTACGACGTGCTGGACAAGCGGAGAGGACGGGACCCAGAAATGGGCGG |
| | | GAAGCCGCGCAGAAAGAATCCCCAAGAGGGCCTGTACAACGAGCTCCAA |
| | | AAGGATAAGATGGCAGAAGCCTATAGCGAGATTGGTATGAAAGGGGAAC |
| | | GCAGAAGAGGCAAAGGCCACGACGGACTGTACCAGGGACTCAGCACCGC |
| | | CACCAAGGACACCTATGACGCTCTTCACATGCAGGCCCTGCCGCCTCGG |

139105

| | | |
|---|---|---|
| 139105-aa | 40 | QVQLVESGGGLVQPGRSLRLSCAASGFTFDDYAMHWVRQAPGKGLEWVS |
| ScFv domain | | GISWNSGSIGYADSVKGRFTISRDNAKNSLYLQMNSLRAEDTALYYCSV |
| | | HSFLAYWGQGTLVTVSSASGGGGSGGRASGGGGSDIVMTQTPLSLPVTP |
| | | GEPASISCRSSQSLLHSNGYNYLDWYLQKPGQSPQLLIYLGSNRASGVP |
| | | DRFSGSGSGTDFTLKISRVEAEDVGVYYCMQALQTPYTFGQGTKVEIK |
| 139105-nt | 55 | CAAGTGCAACTCGTCGAATCCGGTGGAGGTCTGGTCCAACCTGGTAGAA |
| ScFv domain | | GCCTGAGACTGTCGTGTGCGGCCAGCGGATTCACCTTTGATGACTATGC |
| | | TATGCACTGGGTGCGGCAGGCCCCAGGAAAGGGCCTGGAATGGGTGTCG |
| | | GGAATTAGCTGGAACTCCGGGTCCATTGGCTACGCCGACTCCGTGAAGG |
| | | GCCGCTTCACCATCTCCCGCGACAACGCAAAGAACTCCCTGTACTTGCA |
| | | AATGAACTCGCTCAGGGCTGAGGATACCGCGCTGTACTACTGCTCCGTG |
| | | CATTCCTTCCTGGCCTACTGGGGACAGGGAACTCTGGTCACCGTGTCGA |
| | | GCGCCTCCGGCGGCGGGGGCTCGGGTGGACGGGCCTCGGGCGGAGGGGG |
| | | GTCCGACATCGTGATGACCCAGACCCCGCTGAGCTTGCCCGTGACTCCC |
| | | GGAGAGCCTGCATCCATCTCCTGCCGGTCATCCCAGTCCCTTCTCCACT |
| | | CCAACGGATACAACTACCTCGACTGGTACCTCCAGAAGCCGGGACAGAG |
| | | CCCTCAGCTTCTGATCTACCTGGGGTCAAATAGAGCCTCAGGAGTGCCG |
| | | GATCGGTTCAGCGGATCTGGTTCGGGAACTGATTTCACTCTGAAGATTT |
| | | CCCGCGTGGAAGCCGAGGACGTGGGCGTCTACTACTGTATGCAGGCGCT |
| | | GCAGACCCCCTATACCTTCGGCCAAGGGACGAAAGTGGAGATCAAG |
| 139105-aa | 70 | QVQLVESGGGLVQPGRSLRLSCAASGFTFDDYAMHWVRQAPGKGLEWVS |
| VH | | GISWNSGSIGYADSVKGRFTISRDNAKNSLYLQMNSLRAEDTALYYCSV |
| | | HSFLAYWGQGTLVTVSS |
| 139105-aa | 85 | DIVMTQTPLSLPVTPGEPASISCRSSQSLLHSNGYNYLDWYLQKPGQSP |
| VL | | QLLIYLGSNRASGVPDRFSGSGSGTDFTLKISRVEAEDVGVYYCMQALQ |
| | | TPYTFGQGTKVEIK |
| 139105-aa | 100 | MALPVTALLLPLALLLHAARPQVQLVESGGGLVQPGRSLRLSCAASGFT |
| Full CAR | | FDDYAMHWVRQAPGKGLEWVSGISWNSGSIGYADSVKGRFTISRDNAKN |
| | | SLYLQMNSLRAEDTALYYCSVHSFLAYWGQGTLVTVSSASGGGGSGGRA |
| | | SGGGGSDIVMTQTPLSLPVTPGEPASISCRSSQSLLHSNGYNYLDWYLQ |
| | | KPGQSPQLLIYLGSNRASGVPDRFSGSGSGTDFTLKISRVEAEDVGVYY |
| | | CMQALQTPYTFGQGTKVEIKTTTPAPRPPTPAPTIASQPLSLRPEACRP |
| | | AAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| 139105-nt | 115 | ATGGCCCTCCCTGTCACCGCCCTGCTGCTTCCGCTGGCTCTTCTGCTCC |
| Full CAR | | ACGCCGCTCGGCCCCAAGTGCAACTCGTCGAATCCGGTGGAGGTCTGGT |
| | | CCAACCTGGTAGAAGCCTGAGACTGTCGTGTGCGGCCAGCGGATTCACC |
| | | TTTGATGACTATGCTATGCACTGGGTGCGGCAGGCCCCAGGAAAGGGCC |
| | | TGGAATGGGTGTCGGGAATTAGCTGGAACTCCGGGTCCATTGGCTACGC |
| | | CGACTCCGTGAAGGGCCGCTTCACCATCTCCCGCGACAACGCAAAGAAC |
| | | TCCCTGTACTTGCAAATGAACTCGCTCAGGGCTGAGGATACCGCGCTGT |
| | | ACTACTGCTCCGTGCATTCCTTCCTGGCCTACTGGGGACAGGGAACTCT |
| | | GGTCACCGTGTCGAGCGCCTCCGGCGGCGGGGGCTCGGGTGGACGGGCC |
| | | TCGGGCGGAGGGGGGTCCGACATCGTGATGACCCAGACCCCGCTGAGCT |
| | | TGCCCGTGACTCCCGGAGAGCCTGCATCCATCTCCTGCCGGTCATCCCA |
| | | GTCCCTTCTCCACTCCAACGGATACAACTACCTCGACTGGTACCTCCAG |
| | | AAGCCGGGACAGAGCCCTCAGCTTCTGATCTACCTGGGGTCAAATAGAG |
| | | CCTCAGGAGTGCCGGATCGGTTCAGCGGATCTGGTTCGGGAACTGATTT |
| | | CACTCTGAAGATTTCCCGCGTGGAAGCCGAGGACGTGGGCGTCTACTAC |
| | | TGTATGCAGGCGCTGCAGACCCCCTATACCTTCGGCCAAGGGACGAAAG |
| | | TGGAGATCAAGACCACTACCCCAGCACCGAGGCCACCCACCCCGGCTCC |
| | | TACCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCGATA |
| | | TCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTAC |
| | | ATCTTTAAGCAACCCTTCATGAGGCCTGTGCAGACTACTCAAGAGGAGG |
| | | ACGGCTGTTCATGCCGGTTCCCAGAGGAGGAGGAAGGCGGCTGCGAACT |
| | | GCGCGTGAAATTCAGCCGCAGCGCAGATGCTCCAGCCTACAAGCAGGGG |
| | | CAGAACCAGCTCTACAACGAACTCAATCTTGGTCGGAGAGAGGAGTACG |
| | | ACGTGCTGGACAAGCGGAGAGGACGGGACCCAGAAATGGGCGGGAAGCC |
| | | GCGCAGAAAGAATCCCCAAGAGGGCCTGTACAACGAGCTCCAAAAGGAT |
| | | AAGATGGCAGAAGCCTATAGCGAGATTGGTATGAAAGGGGAACGCAGAA |
| | | GAGGCAAAGGCCACGACGGACTGTACCAGGGACTCAGCACCGCCACCAA |
| | | GGACACCTATGACGCTCTTCACATGCAGGCCCTGCCGCCTCGG |

139111

| | | |
|---|---|---|
| 139111-aa | 41 | EVQLLESGGGLVQPGGSLRLSCAVSGFALSNHGMSWVRRAPGKGLEWVS |
| ScFv domain | | GIVYSGSTYYAASVKGRFTISRDNSRNTLYLQMNSLRPEDTAIYYCSAH |
| | | GGESDVWGQGTTVTVSSASGGGGSGGRASGGGGSDIVMTQTPLSLSVTP |
| | | GQPASISCKSSQSLLRNDGKTPLYWYLQKAGQPPQLLIYEVSNRFSGVP |
| | | DRFSGSGSGTDFTLKISRVEAEDVGAYYCMQNIQFPSFGGGTKLEIK |
| 139111-nt | 56 | GAAGTGCAATTGTTGGAATCTGGAGGAGGACTTGTGCAGCCTGGAGGAT |
| ScFv domain | | CACTGAGACTTTCGTGTGCGGTGTCAGGCTTCGCCCTGAGCAACCACGG |
| | | CATGAGCTGGGTGCGGAGAGCCCCGGGGAAGGGTCTGGAATGGGTGTCC |
| | | GGGATCGTCTACTCCGGTTCAACTTACTACGCCGCAAGCGTGAAGGGTC |
| | | GCTTCACCATTTCCCGCGATAACTCCCGGAACACCCTGTACCTCCAAAT |
| | | GAACTCCCTGCGGCCCGAGGACACCGCCATCTACTACTGTTCCGCGCAT |
| | | GGAGGAGAGTCCGATGTCTGGGGACAGGGCACTACCGTGACCGTGTCGA |
| | | GCGCCTCGGGGGGAGGAGGCTCCGGCGGTCGCGCCTCCGGGGGGGGTGG |
| | | CAGCGACATTGTGATGACGCAGACTCCACTCTCGCTGTCCGTGACCCCG |
| | | GGACAGCCCGCGTCCATCTCGTGCAAGAGCTCCCAGAGCCTGCTGAGGA |
| | | ACGACGGAAAGACTCCTCTGTATTGGTACCTCCAGAAGGCTGGACAGCC |
| | | CCCGCAACTGCTCATCTACGAAGTGTCAAATCGCTTCTCCGGGGTGCCG |
| | | GATCGGTTTTCCGGCTCGGGATCGGGCACCGACTTCACCCTGAAAATCT |
| | | CCAGGGTCGAGGCCGAGGACGTGGGAGCCTACTACTGCATGCAAAACAT |
| | | CCAGTTCCCTTCCTTCGGCGGCGGCACAAAGCTGGAGATTAAG |
| 139111-aa | 71 | EVQLLESGGGLVQPGGSLRLSCAVSGFALSNHGMSWVRRAPGKGLEWVS |
| VH | | GIVYSGSTYYAASVKGRFTISRDNSRNTLYLQMNSLRPEDTAIYYCSAH |
| | | GGESDVWGQGTTVTVSS |
| 139111-aa | 86 | DIVMTQTPLSLSVTPGQPASISCKSSQSLLRNDGKTPLYWYLQKAGQPP |
| VL | | QLLIYEVSNRFSGVPDRFSGSGSGTDFTLKISRVEAEDVGAYYCMQNIQ |
| | | FPSFGGGTKLEIK |
| 139111-aa | 101 | MALPVTALLLPLALLLHAARPEVQLLESGGGLVQPGGSLRLSCAVSGFA |
| Full CAR | | LSNHGMSWVRRAPGKGLEWVSGIVYSGSTYYAASVKGRFTISRDNSRNT |
| | | LYLQMNSLRPEDTAIYYCSAHGGESDVWGQGTTVTVSSASGGGGSGGRA |
| | | SGGGGSDIVMTQTPLSLSVTPGQPASISCKSSQSLLRNDGKTPLYWYLQ |
| | | KAGQPPQLLIYEVSNRFSGVPDRFSGSGSGTDFTLKISRVEAEDVGAYY |
| | | CMQNIQFPSFGGGTKLEIKTTTPAPRPPTPAPTIASQPLSLRPEACRPA |
| | | AGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 139111-nt | 116 | ATGGCCCTCCCTGTCACCGCCCTGCTGCTTCCGCTGGCTCTTCTGCTCC |
| Full CAR | | ACGCCGCTCGGCCCGAAGTGCAATTGTTGGAATCTGGAGGAGGACTTGT |
| | | GCAGCCTGGAGGATCACTGAGACTTTCGTGTGCGGTGTCAGGCTTCGCC |
| | | CTGAGCAACCACGGCATGAGCTGGGTGCGGAGAGCCCCGGGGAAGGGTC |
| | | TGGAATGGGTGTCCGGGATCGTCTACTCCGGTTCAACTTACTACGCCGC |
| | | AAGCGTGAAGGGTCGCTTCACCATTTCCCGCGATAACTCCCGGAACACC |
| | | CTGTACCTCCAAATGAACTCCCTGCGGCCCGAGGACACCGCCATCTACT |
| | | ACTGTTCCGCGCATGGAGGAGAGTCCGATGTCTGGGGACAGGGCACTAC |
| | | CGTGACCGTGTCGAGCGCCTCGGGGGGAGGAGGCTCCGGCGGTCGCGCC |
| | | TCCGGGGGGGGTGGCAGCGACATTGTGATGACGCAGACTCCACTCTCGC |
| | | TGTCCGTGACCCCGGGACAGCCCGCGTCCATCTCGTGCAAGAGCTCCCA |
| | | GAGCCTGCTGAGGAACGACGGAAAGACTCCTCTGTATTGGTACCTCCAG |
| | | AAGGCTGGACAGCCCCCGCAACTGCTCATCTACGAAGTGTCAAATCGCT |
| | | TCTCCGGGGTGCCGGATCGGTTTTCCGGCTCGGGATCGGGCACCGACTT |
| | | CACCCTGAAAATCTCCAGGGTCGAGGCCGAGGACGTGGGAGCCTACTAC |
| | | TGCATGCAAAACATCCAGTTCCCTTCCTTCGGCGGCGGCACAAAGCTGG |
| | | AGATTAAGACCACTACCCCAGCACCGAGGCCACCCACCCCGGCTCCTAC |
| | | CATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCT |
| | | ACATTTGGGCCCCTCTGGCTGGTACTTGCGGGGTCCTGCTGCTTTCACT |
| | | CGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACG |
| | | GCTGTTCATGCCGGTTCCCAGAGGAGGAGGAAGGCGGCTGCGAACTGCG |
| | | CGTGAAATTCAGCCGCAGCGCAGATGCTCCAGCCTACAAGCAGGGGCAG |
| | | AACCAGCTCTACAACGAACTCAATCTTGGTCGGAGAGAGGAGTACGACG |
| | | TGCTGGACAAGCGGAGAGGACGGGACCCAGAAATGGGCGGGAAGCCGCG |
| | | CAGAAAGAATCCCCAAGAGGGCCTGTACAACGAGCTCCAAAAGGATAAG |
| | | ATGGCAGAAGCCTATAGCGAGATTGGTATGAAAGGGGAACGCAGAAGAG |
| | | GCAAAGGCCACGACGGACTGTACCAGGGACTCAGCACCGCCACCAAGGA |
| | | CACCTATGACGCTCTTCACATGCAGGCCCTGCCGCCTCGG |

139100

| | | |
|---|---|---|
| 139100-aa | 42 | QVQLVQSGAEVRKTGASVKVSCKASGYIFDNFGINWVRQAPGQGLEWMG |
| ScFv domain | | WINPKNNNTNYAQKFQGRVTITADESTNTAYMEVSSLRSEDTAVYYCAR |
| | | GPYYYQSYMDVWGQGTMVTVSSASGGGGSGGRASGGGGSDIVMTQTPLS |
| | | LPVTPGEPASISCRSSQSLLHSNGYNYLNWYLQKPGQSPQLLIYLGSKR |
| | | ASGVPDRFSGSGSGTDFTLHITRVGAEDVGVYYCMQALQTPYTFGQGTK |
| | | LEIK |
| 139100-nt | 57 | CAAGTCCAACTCGTCCAGTCCGGCGCAGAAGTCAGAAAAACCGGTGCTA |
| ScFv domain | | GCGTGAAAGTGTCCTGCAAGGCCTCCGGCTACATTTTCGATAACTTCGG |
| | | AATCAACTGGGTCAGACAGGCCCCGGGCCAGGGGCTGGAATGGATGGGA |
| | | TGGATCAACCCCAAGAACAACAACACCAACTACGCACAGAAGTTCCAGG |
| | | GCCGCGTGACTATCACCGCCGATGAATCGACCAATACCGCCTACATGGA |
| | | GGTGTCCTCCCTGCGGTCGGAGGACACTGCCGTGTATTACTGCGCGAGG |
| | | GGCCCATACTACTACCAAAGCTACATGGACGTCTGGGGACAGGGAACCA |
| | | TGGTGACCGTGTCATCCGCCTCCGGTGGTGGAGGCTCCGGGGGGCGGGC |
| | | TTCAGGAGGCGGAGGAAGCGATATTGTGATGACCCAGACTCCGCTTAGC |
| | | CTGCCCGTGACTCCTGGAGAACCGGCCTCCATTTCCTGCCGGTCCTCGC |
| | | AATCACTCCTGCATTCCAACGGTTACAACTACCTGAATTGGTACCTCCA |
| | | GAAGCCTGGCCAGTCGCCCCAGTTGCTGATCTATCTGGGCTCGAAGCGC |
| | | GCCTCCGGGGTGCCTGACCGGTTTAGCGGATCTGGGAGCGGCACGGACT |
| | | TCACTCTCCACATCACCCGCGTGGGAGCGGAGGACGTGGGAGTGTACTA |
| | | CTGTATGCAGGCGCTGCAGACTCCGTACACATTCGGACAGGGCACCAAG |
| | | CTGGAGATCAAG |
| 139100-aa | 72 | QVQLVQSGAEVRKTGASVKVSCKASGYIFDNFGINWVRQAPGQGLEWMG |
| VH | | WINPKNNNTNYAQKFQGRVTITADESTNTAYMEVSSLRSEDTAVYYCAR |
| | | GPYYYQSYMDVWGQGTMVTVSS |
| 139100-aa | 87 | DIVMTQTPLSLPVTPGEPASISCRSSQSLLHSNGYNYLNWYLQKPGQSP |
| VL | | QLLIYLGSKRASGVPDRFSGSGSGTDFTLHITRVGAEDVGVYYCMQALQ |
| | | TPYTFGQGTKLEIK |
| 139100-aa | 102 | MALPVTALLLPLALLLHAARPQVQLVQSGAEVRKTGASVKVSCKASGYI |
| Full CAR | | FDNFGINWVRQAPGQGLEWMGWINPKNNNTNYAQKFQGRVTITADESTN |
| | | TAYMEVSSLRSEDTAVYYCARGPYYYQSYMDVWGQGTMVTVSSASGGGG |
| | | SGGRASGGGGSDIVMTQTPLSLPVTPGEPASISCRSSQSLLHSNGYNYL |
| | | NWYLQKPGQSPQLLIYLGSKRASGVPDRFSGSGSGTDFTLHITRVGAED |
| | | VGVYYCMQALQTPYTFGQGTKLEIKTTTPAPRPPTPAPTIASQPLSLRP |
| | | EACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAP |
| | | AYKQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQAL |
| | | PPR |
| 139100-nt | 117 | ATGGCCCTCCCTGTCACCGCCCTGCTGCTTCCGCTGGCTCTTCTGCTCC |
| Full CAR | | ACGCCGCTCGGCCCCAAGTCCAACTCGTCCAGTCCGGCGCAGAAGTCAG |
| | | AAAAACCGGTGCTAGCGTGAAAGTGTCCTGCAAGGCCTCCGGCTACATT |
| | | TTCGATAACTTCGGAATCAACTGGGTCAGACAGGCCCCGGGCCAGGGGC |
| | | TGGAATGGATGGGATGGATCAACCCCAAGAACAACAACACCAACTACGC |
| | | ACAGAAGTTCCAGGGCCGCGTGACTATCACCGCCGATGAATCGACCAAT |
| | | ACCGCCTACATGGAGGTGTCCTCCCTGCGGTCGGAGGACACTGCCGTGT |
| | | ATTACTGCGCGAGGGGCCCATACTACTACCAAAGCTACATGGACGTCTG |
| | | GGGACAGGGAACCATGGTGACCGTGTCATCCGCCTCCGGTGGTGGAGGC |
| | | TCCGGGGGGCGGGCTTCAGGAGGCGGAGGAAGCGATATTGTGATGACCC |
| | | AGACTCCGCTTAGCCTGCCCGTGACTCCTGGAGAACCGGCCTCCATTTC |
| | | CTGCCGGTCCTCGCAATCACTCCTGCATTCCAACGGTTACAACTACCTG |
| | | AATTGGTACCTCCAGAAGCCTGGCCAGTCGCCCCAGTTGCTGATCTATC |
| | | TGGGCTCGAAGCGCGCCTCCGGGGTGCCTGACCGGTTTAGCGGATCTGG |
| | | GAGCGGCACGGACTTCACTCTCCACATCACCCGCGTGGGAGCGGAGGAC |
| | | GTGGGAGTGTACTACTGTATGCAGGCGCTGCAGACTCCGTACACATTCG |
| | | GACAGGGCACCAAGCTGGAGATCAAGACCACTACCCCAGCACCGAGGCC |
| | | ACCCACCCCGGCTCCTACCATCGCCTCCCAGCCTCTGTCCCTGCGTCCG |
| | | GAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGG |
| | | GGTCCTGCTGCTTTCACTCGTGATCACTCTTTACTGTAAGCGCGGTCGG |
| | | AAGAAGCTGCTGTACATCTTTAAGCAACCCTTCATGAGGCCTGTGCAGA |
| | | CTACTCAAGAGGAGGACGGCTGTTCATGCCGGTTCCCAGAGGAGGAGGA |
| | | AGGCGGCTGCGAACTGCGCGTGAAATTCAGCCGCAGCGCAGATGCTCCA |
| | | GCCTACAAGCAGGGGCAGAACCAGCTCTACAACGAACTCAATCTTGGTC |
| | | GGAGAGAGGAGTACGACGTGCTGGACAAGCGGAGAGGACGGGACCCAGA |
| | | AATGGGCGGGAAGCCGCGCAGAAAGAATCCCCAAGAGGGCCTGTACAAC |
| | | GAGCTCCAAAAGGATAAGATGGCAGAAGCCTATAGCGAGATTGGTATGA |
| | | AAGGGGAACGCAGAAGAGGCAAAGGCCACGACGGACTGTACCAGGGACT |
| | | CAGCACCGCCACCAAGGACACCTATGACGCTCTTCACATGCAGGCCCTG |
| | | CCGCCTCGG |

139101

| | | |
|---|---|---|
| 139101-aa | 43 | QVQLQESGGGLVQPGGSLRLSCAASGFTFSSDAMTWVRQAPGKGLEWVS |
| ScFv domain | | VISGSGGTTYYADSVKGRFTISRDNSKNTLYLQMNSLRAEDTAVYYCAK |
| | | LDSSGYYYARGPRYWGQGTLVTVSSASGGGGSGGRASGGGGSDIQLTQS |
| | | PSSLSASVGDRVTITCRASQSISSYLNWYQQKPGKAPKLLIYGASTLAS |
| | | GVPARFSGSGSGTHFTLTINSLQSEDSATYYCQQSYKRASFGQGTKVEI |
| | | K |
| 139101-nt | 58 | CAAGTGCAACTTCAAGAATCAGGCGGAGGACTCGTGCAGCCCGGAGGAT |
| ScFv domain | | CATTGCGGCTCTCGTGCGCCGCCTCGGGCTTCACCTTCTCGAGCGACGC |
| | | CATGACCTGGGTCCGCCAGGCCCCGGGGAAGGGGCTGGAATGGGTGTCT |
| | | GTGATTTCCGGCTCCGGGGGAACTACGTACTACGCCGATTCCGTGAAAG |
| | | GTCGCTTCACTATCTCCCGGGACAACAGCAAGAACACCCTTTATCTGCA |
| | | AATGAATTCCCTCCGCGCCGAGGACACCGCCGTGTACTACTGCGCCAAG |
| | | CTGGACTCCTCGGGCTACTACTATGCCCGGGGTCCGAGATACTGGGGAC |
| | | AGGGAACCCTCGTGACCGTGTCCTCCGCGTCCGGCGGAGGAGGGTCGGG |
| | | AGGGCGGGCCTCCGGCGGCGGCGGTTCGGACATCCAGCTGACCCAGTCC |
| | | CCATCCTCACTGAGCGCAAGCGTGGGCGACAGAGTCACCATTACATGCA |
| | | GGGCGTCCCAGAGCATCAGCTCCTACCTGAACTGGTACCAACAGAAGCC |
| | | TGGAAAGGCTCCTAAGCTGTTGATCTACGGGGCTTCGACCCTGGCATCC |
| | | GGGGTGCCCGCGAGGTTTAGCGGAAGCGGTAGCGGCACTCACTTCACTC |
| | | TGACCATTAACAGCCTCCAGTCCGAGGATTCAGCCACTTACTACTGTCA |
| | | GCAGTCCTACAAGCGGGCCAGCTTCGGACAGGGCACTAAGGTCGAGATC |
| | | AAG |
| 139101-aa | 73 | QVQLQESGGGLVQPGGSLRLSCAASGFTFSSDAMTWVRQAPGKGLEWVS |
| VH | | VISGSGGTTYYADSVKGRFTISRDNSKNTLYLQMNSLRAEDTAVYYCAK |
| | | LDSSGYYYARGPRYWGQGTLVTVSS |
| 139101-aa | 88 | DIQLTQSPSSLSASVGDRVTITCRASQSISSYLNWYQQKPGKAPKLLIY |
| VL | | GASTLASGVPARFSGSGSGTHFTLTINSLQSEDSATYYCQQSYKRASFG |
| | | QGTKVEIK |
| 139101-aa | 103 | MALPVTALLLPLALLLHAARPQVQLQESGGGLVQPGGSLRLSCAASGFT |
| Full CAR | | FSSDAMTWVRQAPGKGLEWVSVISGSGGTTYYADSVKGRFTISRDNSKN |
| | | TLYLQMNSLRAEDTAVYYCAKLDSSGYYYARGPRYWGQGTLVTVSSASG |
| | | GGGSGGRASGGGGSDIQLTQSPSSLSASVGDRVTITCRASQSISSYLNW |
| | | YQQKPGKAPKLLIYGASTLASGVPARFSGSGSGTHFTLTINSLQSEDSA |
| | | TYYCQQSYKRASFGQGTKVEIKTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 139101-nt | 118 | ATGGCCCTCCCTGTCACCGCCCTGCTGCTTCCGCTGGCTCTTCTGCTCC |
| Full CAR | | ACGCCGCTCGGCCCCAAGTGCAACTTCAAGAATCAGGCGGAGGACTCGT |
| | | GCAGCCCGGAGGATCATTGCGGCTCTCGTGCGCCGCCTCGGGCTTCACC |
| | | TTCTCGAGCGACGCCATGACCTGGGTCCGCCAGGCCCCGGGGAAGGGGC |
| | | TGGAATGGGTGTCTGTGATTTCCGGCTCCGGGGGAACTACGTACTACGC |
| | | CGATTCCGTGAAAGGTCGCTTCACTATCTCCCGGGACAACAGCAAGAAC |
| | | ACCCTTTATCTGCAAATGAATTCCCTCCGCGCCGAGGACACCGCCGTGT |
| | | ACTACTGCGCCAAGCTGGACTCCTCGGGCTACTACTATGCCCGGGGTCC |
| | | GAGATACTGGGGACAGGGAACCCTCGTGACCGTGTCCTCCGCGTCCGGC |
| | | GGAGGAGGGTCGGGAGGGCGGGCCTCCGGCGGCGGCGGTTCGGACATCC |
| | | AGCTGACCCAGTCCCCATCCTCACTGAGCGCAAGCGTGGGCGACAGAGT |
| | | CACCATTACATGCAGGGCGTCCCAGAGCATCAGCTCCTACCTGAACTGG |
| | | TACCAACAGAAGCCTGGAAAGGCTCCTAAGCTGTTGATCTACGGGGCTT |
| | | CGACCCTGGCATCCGGGGTGCCCGCGAGGTTTAGCGGAAGCGGTAGCGG |
| | | CACTCACTTCACTCTGACCATTAACAGCCTCCAGTCCGAGGATTCAGCC |
| | | ACTTACTACTGTCAGCAGTCCTACAAGCGGGCCAGCTTCGGACAGGGCA |
| | | CTAAGGTCGAGATCAAGACCACTACCCCAGCACCGAGGCCACCCACCCC |
| | | GGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAG |
| | | AGGAGGACGGCTGTTCATGCCGGTTCCCAGAGGAGGAGGAAGGCGGCTG |
| | | CGAACTGCGCGTGAAATTCAGCCGCAGCGCAGATGCTCCAGCCTACAAG |
| | | CAGGGGCAGAACCAGCTCTACAACGAACTCAATCTTGGTCGGAGAGAGG |
| | | AGTACGACGTGCTGGACAAGCGGAGAGGACGGGACCCAGAAATGGGCGG |
| | | GAAGCCGCGCAGAAAGAATCCCCAAGAGGGCCTGTACAACGAGCTCCAA |
| | | AAGGATAAGATGGCAGAAGCCTATAGCGAGATTGGTATGAAAGGGGAAC |
| | | GCAGAAGAGGCAAAGGCCACGACGGACTGTACCAGGGACTCAGCACCGC |
| | | CACCAAGGACACCTATGACGCTCTTCACATGCAGGCCCTGCCGCCTCGG |

139102

| | | |
|---|---|---|
| 139102-aa | 44 | QVQLVQSGAEVKKPGASVKVSCKASGYTFSNYGITWVRQAPGQGLEWMG |
| ScFv domain | | WISAYNGNTNYAQKFQGRVTMTRNTSISTAYMELSSLRSEDTAVYYCAR |
| | | GPYYYYMDVWGKGTMVTVSSASGGGGSGGRASGGGGSEIVMTQSPLSLP |
| | | VTPGEPASISCRSSQSLLYSNGYNYVDWYLQKPGQSPQLLIYLGSNRAS |
| | | GVPDRFSGSGSGTDFKLQISRVEAEDVGIYYCMQGRQFPYSFGQGTKVE |
| | | IK |
| 139102-nt | 59 | CAAGTCCAACTGGTCCAGAGCGGTGCAGAAGTGAAGAAGCCCGGAGCGA |
| ScFv domain | | GCGTGAAAGTGTCCTGCAAGGCTTCCGGGTACACCTTCTCCAACTACGG |
| | | CATCACTTGGGTGCGCCAGGCCCCGGGACAGGGCCTGGAATGGATGGGG |
| | | TGGATTTCCGCGTACAACGGCAATACGAACTACGCTCAGAAGTTCCAGG |
| | | GTAGAGTGACCATGACTAGGAACACCTCCATTTCCACCGCCTACATGGA |
| | | ACTGTCCTCCCTGCGGAGCGAGGACACCGCCGTGTACTATTGCGCCCGG |
| | | GGACCATACTACTACTACATGGATGTCTGGGGGAAGGGGACTATGGTCA |
| | | CCGTGTCATCCGCCTCGGGAGGCGGCGGATCAGGAGGACGCGCCTCTGG |
| | | TGGTGGAGGATCGGAGATCGTGATGACCCAGAGCCCTCTCTCCTTGCCC |
| | | GTGACTCCTGGGGAGCCCGCATCCATTTCATGCCGGAGCTCCCAGTCAC |
| | | TTCTCTACTCCAACGGCTATAACTACGTGGATTGGTACCTCCAAAAGCC |
| | | GGGCCAGAGCCCGCAGCTGCTGATCTACCTGGGCTCGAACAGGGCCAGC |
| | | GGAGTGCCTGACCGGTTCTCCGGGTCGGGAAGCGGGACCGACTTCAAGC |
| | | TGCAAATCTCGAGAGTGGAGGCCGAGGACGTGGGAATCTACTACTGTAT |
| | | GCAGGGCCGCCAGTTTCCGTACTCGTTCGGACAGGGCACCAAAGTGGAA |
| | | ATCAAG |
| 139102-aa | 74 | QVQLVQSGAEVKKPGASVKVSCKASGYTFSNYGITWVRQAPGQGLEWMG |
| VH | | WISAYNGNTNYAQKFQGRVTMTRNTSISTAYMELSSLRSEDTAVYYCAR |
| | | GPYYYYMDVWGKGTMVTVSS |
| 139102-aa | 89 | EIVMTQSPLSLPVTPGEPASISCRSSQSLLYSNGYNYVDWYLQKPGQSP |
| VL | | QLLIYLGSNRASGVPDRFSGSGSGTDFKLQISRVEAEDVGIYYCMQGRQ |
| | | FPYSFGQGTKVEIK |
| 139102-aa | 104 | MALPVTALLLPLALLLHAARPQVQLVQSGAEVKKPGASVKVSCKASGYT |
| Full CAR | | FSNYGITWVRQAPGQGLEWMGWISAYNGNTNYAQKFQGRVTMTRNTSIS |
| | | TAYMELSSLRSEDTAVYYCARGPYYYYMDVWGKGTMVTVSSASGGGGSG |
| | | GRASGGGGSEIVMTQSPLSLPVTPGEPASISCRSSQSLLYSNGYNYVDW |
| | | YLQKPGQSPQLLIYLGSNRASGVPDRFSGSGSGTDFKLQISRVEAEDVG |
| | | IYYCMQGRQFPYSFGQGTKVEIKTTTPAPRPPTPAPTIASQPLSLRPEA |
| | | CRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPP |
| | | R |
| 139102-nt | 119 | ATGGCCCTCCCTGTCACCGCCCTGCTGCTTCCGCTGGCTCTTCTGCTCC |
| Full CAR | | ACGCCGCTCGGCCCCAAGTCCAACTGGTCCAGAGCGGTGCAGAAGTGAA |
| | | GAAGCCCGGAGCGAGCGTGAAAGTGTCCTGCAAGGCTTCCGGGTACACC |
| | | TTCTCCAACTACGGCATCACTTGGGTGCGCCAGGCCCCGGGACAGGGCC |
| | | TGGAATGGATGGGGTGGATTTCCGCGTACAACGGCAATACGAACTACGC |
| | | TCAGAAGTTCCAGGGTAGAGTGACCATGACTAGGAACACCTCCATTTCC |
| | | ACCGCCTACATGGAACTGTCCTCCCTGCGGAGCGAGGACACCGCCGTGT |
| | | ACTATTGCGCCCGGGGACCATACTACTACTACATGGATGTCTGGGGGAA |
| | | GGGGACTATGGTCACCGTGTCATCCGCCTCGGGAGGCGGCGGATCAGGA |
| | | GGACGCGCCTCTGGTGGTGGAGGATCGGAGATCGTGATGACCCAGAGCC |
| | | CTCTCTCCTTGCCCGTGACTCCTGGGGAGCCCGCATCCATTTCATGCCG |
| | | GAGCTCCCAGTCACTTCTCTACTCCAACGGCTATAACTACGTGGATTGG |
| | | TACCTCCAAAAGCCGGGCCAGAGCCCGCAGCTGCTGATCTACCTGGGCT |
| | | CGAACAGGGCCAGCGGAGTGCCTGACCGGTTCTCCGGGTCGGGAAGCGG |
| | | GACCGACTTCAAGCTGCAAATCTCGAGAGTGGAGGCCGAGGACGTGGGA |
| | | ATCTACTACTGTATGCAGGGCCGCCAGTTTCCGTACTCGTTCGGACAGG |
| | | GCACCAAAGTGGAAATCAAGACCACTACCCCAGCACCGAGGCCACCCAC |
| | | CCCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCT |
| | | GCTGCTTTCACTCGTGATCACTCTTTACTGTAAGCGCGGTCGGAAGAAG |
| | | CTGCTGTACATCTTTAAGCAACCCTTCATGAGGCCTGTGCAGACTACTC |
| | | AAGAGGAGGACGGCTGTTCATGCCGGTTCCCAGAGGAGGAGGAAGGCGG |
| | | CTGCGAACTGCGCGTGAAATTCAGCCGCAGCGCAGATGCTCCAGCCTAC |
| | | AAGCAGGGGCAGAACCAGCTCTACAACGAACTCAATCTTGGTCGGAGAG |
| | | AGGAGTACGACGTGCTGGACAAGCGGAGAGGACGGGACCCAGAAATGGG |
| | | CGGGAAGCCGCGCAGAAAGAATCCCCAAGAGGGCCTGTACAACGAGCTC |
| | | CAAAAGGATAAGATGGCAGAAGCCTATAGCGAGATTGGTATGAAAGGGG |
| | | AACGCAGAAGAGGCAAAGGCCACGACGGACTGTACCAGGGACTCAGCAC |
| | | CGCCACCAAGGACACCTATGACGCTCTTCACATGCAGGCCCTGCCGCCT |
| | | CGG |

139104

| | | |
|---|---|---|
| 139104-aa | 45 | EVQLLETGGGLVQPGGSLRLSCAVSGFALSNHGMSWVRRAPGKGLEWVS |
| ScFv domain | | GIVYSGSTYYAASVKGRFTISRDNSRNTLYLQMNSLRPEDTAIYYCSAH |
| | | GGESDVWGQGTTVTVSSASGGGGSGGRASGGGGSEIVLTQSPATLSVSP |
| | | GESATLSCRASQSVSSNLAWYQQKPGQAPRLLIYGASTRASGIPDRFSG |
| | | SGSGTDFTLTISSLQAEDVAVYYCQQYGSSLTFGGGTKVEIK |
| 139104-nt | 60 | GAAGTGCAATTGCTCGAAACTGGAGGAGGTCTGGTGCAACCTGGAGGAT |
| ScFv domain | | CACTTCGCCTGTCCTGCGCCGTGTCGGGCTTTGCCCTGTCCAACCATGG |
| | | AATGAGCTGGGTCCGCCGCGCGCCGGGGAAGGGCCTCGAATGGGTGTCC |
| | | GGCATCGTCTACTCCGGCTCCACCTACTACGCCGCGTCCGTGAAGGGCC |
| | | GGTTCACGATTTCACGGGACAACTCGCGGAACACCCTGTACCTCCAAAT |
| | | GAATTCCCTTCGGCCGGAGGATACTGCCATCTACTACTGCTCCGCCCAC |
| | | GGTGGCGAATCCGACGTCTGGGGCCAGGGAACCACCGTGACCGTGTCCA |
| | | GCGCGTCCGGGGGAGGAGGAAGCGGGGGTAGAGCATCGGGTGGAGGCGG |
| | | ATCAGAGATCGTGCTGACCCAGTCCCCCGCCACCTTGAGCGTGTCACCA |
| | | GGAGAGTCCGCCACCCTGTCATGCCGCGCCAGCCAGTCCGTGTCCTCCA |
| | | ACCTGGCTTGGTACCAGCAGAAGCCGGGGCAGGCCCCTAGACTCCTGAT |
| | | CTATGGGGCGTCGACCCGGGCATCTGGAATTCCCGATAGGTTCAGCGGA |
| | | TCGGGCTCGGGCACTGACTTCACTCTGACCATCTCCTCGCTGCAAGCCG |
| | | AGGACGTGGCTGTGTACTACTGTCAGCAGTACGGAAGCTCCCTGACTTT |
| | | CGGTGGCGGGACCAAAGTCGAGATTAAG |
| 139104-aa | 75 | EVQLLETGGGLVQPGGSLRLSCAVSGFALSNHGMSWVRRAPGKGLEWVS |
| VH | | GIVYSGSTYYAASVKGRFTISRDNSRNTLYLQMNSLRPEDTAIYYCSAH |
| | | GGESDVWGQGTTVTVSS |
| 139104-aa | 90 | EIVLTQSPATLSVSPGESATLSCRASQSVSSNLAWYQQKPGQAPRLLIY |
| VL | | GASTRASGIPDRFSGSGSGTDFTLTISSLQAEDVAVYYCQQYGSSLTFG |
| | | GGTKVEIK |
| 139104-aa | 105 | MALPVTALLLPLALLLHAARPEVQLLETGGGLVQPGGSLRLSCAVSGFA |
| Full CAR | | LSNHGMSWVRRAPGKGLEWVSGIVYSGSTYYAASVKGRFTISRDNSRNT |
| | | LYLQMNSLRPEDTAIYYCSAHGGESDVWGQGTTVTVSSASGGGGSGGRA |
| | | SGGGGSEIVLTQSPATLSVSPGESATLSCRASQSVSSNLAWYQQKPGQA |
| | | PRLLIYGASTRASGIPDRFSGSGSGTDFTLTISSLQAEDVAVYYCQQYG |
| | | SSLTFGGGTKVEIKTTTPAPRPPTPAPTIASQPLSLRPEACRPAAGGAV |
| | | HTRGLDFACDIYIWAPLAGTCGVLLLSLVITLYCKRGRKKLLYIFKQPF |
| | | MRPVQTTQEEDGCSCRFPEEEEGGCELRVKFSRSADAPAYKQGQNQLYN |
| | | ELNLGRREEYDVLDKRRGRDPEMGGKPRRKNPQEGLYNELQKDKMAEAY |
| | | SEIGMKGERRRGKGHDGLYQGLSTATKDTYDALHMQALPPR |
| 139104-nt | 120 | ATGGCCCTCCCTGTCACCGCCCTGCTGCTTCCGCTGGCTCTTCTGCTCC |
| Full CAR | | ACGCCGCTCGGCCCGAAGTGCAATTGCTCGAAACTGGAGGAGGTCTGGT |
| | | GCAACCTGGAGGATCACTTCGCCTGTCCTGCGCCGTGTCGGGCTTTGCC |
| | | CTGTCCAACCATGGAATGAGCTGGGTCCGCCGCGCGCCGGGGAAGGGCC |
| | | TCGAATGGGTGTCCGGCATCGTCTACTCCGGCTCCACCTACTACGCCGC |
| | | GTCCGTGAAGGGCCGGTTCACGATTTCACGGGACAACTCGCGGAACACC |
| | | CTGTACCTCCAAATGAATTCCCTTCGGCCGGAGGATACTGCCATCTACT |
| | | ACTGCTCCGCCCACGGTGGCGAATCCGACGTCTGGGGCCAGGGAACCAC |
| | | CGTGACCGTGTCCAGCGCGTCCGGGGGAGGAGGAAGCGGGGGTAGAGCA |
| | | TCGGGTGGAGGCGGATCAGAGATCGTGCTGACCCAGTCCCCCGCCACCT |
| | | TGAGCGTGTCACCAGGAGAGTCCGCCACCCTGTCATGCCGCGCCAGCCA |
| | | GTCCGTGTCCTCCAACCTGGCTTGGTACCAGCAGAAGCCGGGGCAGGCC |
| | | CCTAGACTCCTGATCTATGGGGCGTCGACCCGGGCATCTGGAATTCCCG |
| | | ATAGGTTCAGCGGATCGGGCTCGGGCACTGACTTCACTCTGACCATCTC |
| | | CTCGCTGCAAGCCGAGGACGTGGCTGTGTACTACTGTCAGCAGTACGGA |
| | | AGCTCCCTGACTTTCGGTGGCGGGACCAAAGTCGAGATTAAGACCACTA |
| | | CCCCAGCACCGAGGCCACCCACCCCGGCTCCTACCATCGCCTCCCAGCC |
| | | TCTGTCCCTGCGTCCGGAGGCATGTAGACCCGCAGCTGGTGGGGCCGTG |
| | | CATACCCGGGGTCTTGACTTCGCCTGCGATATCTACATTTGGGCCCCTC |
| | | TGGCTGGTACTTGCGGGGTCCTGCTGCTTTCACTCGTGATCACTCTTTA |
| | | CTGTAAGCGCGGTCGGAAGAAGCTGCTGTACATCTTTAAGCAACCCTTC |
| | | ATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCG |
| | | CAGCGCAGATGCTCCAGCCTACAAGCAGGGGCAGAACCAGCTCTACAAC |
| | | GAACTCAATCTTGGTCGGAGAGAGGAGTACGACGTGCTGGACAAGCGGA |
| | | GAGGACGGGACCCAGAAATGGGCGGGAAGCCGCGCAGAAAGAATCCCCA |
| | | AGAGGGCCTGTACAACGAGCTCCAAAAGGATAAGATGGCAGAAGCCTAT |
| | | AGCGAGATTGGTATGAAAGGGGAACGCAGAAGAGGCAAAGGCCACGACG |
| | | GACTGTACCAGGGACTCAGCACCGCCACCAAGGACACCTATGACGCTCT |
| | | TCACATGCAGGCCCTGCCGCCTCGG |

139106

| | | |
|---|---|---|
| 139106-aa | 46 | EVQLVETGGGLVQPGGSLRLSCAVSGFALSNHGMSWVRRAPGKGLEWVS |
| ScFv domain | | GIVYSGSTYYAASVKGRFTISRDNSRNTLYLQMNSLRPEDTAIYYCSAH |
| | | GGESDVWGQGTTVTVSSASGGGGSGGRASGGGGSEIVMTQSPATLSVSP |
| | | GERATLSCRASQSVSSKLAWYQQKPGQAPRLLMYGASIRATGIPDRFSG |
| | | SGSGTEFTLTISSLEPEDFAVYYCQQYGSSSWTFGQGTKVEIK |
| 139106-nt | 61 | GAAGTGCAATTGGTGGAAACTGGAGGAGGACTTGTGCAACCTGGAGGAT |
| ScFv domain | | CATTGAGACTGAGCTGCGCAGTGTCGGGATTCGCCCTGAGCAACCATGG |
| | | AATGTCCTGGGTCAGAAGGGCCCCTGGAAAAGGCCTCGAATGGGTGTCA |
| | | GGGATCGTGTACTCCGGTTCCACTTACTACGCCGCCTCCGTGAAGGGGC |
| | | GCTTCACTATCTCACGGGATAACTCCCGCAATACCCTGTACCTCCAAAT |
| | | GAACAGCCTGCGGCCGGAGGATACCGCCATCTACTACTGTTCCGCCCAC |
| | | GGTGGAGAGTCTGACGTCTGGGGCCAGGGAACTACCGTGACCGTGTCCT |
| | | CCGCGTCCGGCGGTGGAGGGAGCGGCGGCCGCGCCAGCGGCGGCGGAGG |
| | | CTCCGAGATCGTGATGACCCAGAGCCCCGCTACTCTGTCGGTGTCGCCC |
| | | GGAGAAAGGGCGACCCTGTCCTGCCGGGCGTCGCAGTCCGTGAGCAGCA |
| | | AGCTGGCTTGGTACCAGCAGAAGCCGGGCCAGGCACCACGCCTGCTTAT |
| | | GTACGGTGCCTCCATTCGGGCCACCGGAATCCCGGACCGGTTCTCGGGG |
| | | TCGGGGTCCGGTACCGAGTTCACACTGACCATTTCCTCGCTCGAGCCCG |
| | | AGGACTTTGCCGTCTATTACTGCCAGCAGTACGGCTCCTCCTCATGGAC |
| | | GTTCGGCCAGGGGACCAAGGTCGAAATCAAG |
| 139106-aa | 76 | EVQLVETGGGLVQPGGSLRLSCAVSGFALSNHGMSWVRRAPGKGLEWVS |
| VH | | GIVYSGSTYYAASVKGRFTISRDNSRNTLYLQMNSLRPEDTAIYYCSAH |
| | | GGESDVWGQGTTVTVSS |
| 139106-aa | 91 | EIVMTQSPATLSVSPGERATLSCRASQSVSSKLAWYQQKPGQAPRLLMY |
| VL | | GASIRATGIPDRFSGSGSGTEFTLTISSLEPEDFAVYYCQQYGSSSWTF |
| | | GQGTKVEIK |
| 139106-aa | 106 | MALPVTALLLPLALLLHAARPEVQLVETGGGLVQPGGSLRLSCAVSGFA |
| Full CAR | | LSNHGMSWVRRAPGKGLEWVSGIVYSGSTYYAASVKGRFTISRDNSRNT |
| | | LYLQMNSLRPEDTAIYYCSAHGGESDVWGQGTTVTVSSASGGGGSGGRA |
| | | SGGGGSEIVMTQSPATLSVSPGERATLSCRASQSVSSKLAWYQQKPGQA |
| | | PRLLMYGASIRATGIPDRFSGSGSGTEFTLTISSLEPEDFAVYYCQQYG |
| | | SSSWTFGQGTKVEIKTTTPAPRPPTPAPTIASQPLSLRPEACRPAAGGA |
| | | VHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 139106-nt | 121 | ATGGCCCTCCCTGTCACCGCCCTGCTGCTTCCGCTGGCTCTTCTGCTCC |
| Full CAR | | ACGCCGCTCGGCCCGAAGTGCAATTGGTGGAAACTGGAGGAGGACTTGT |
| | | GCAACCTGGAGGATCATTGAGACTGAGCTGCGCAGTGTCGGGATTCGCC |
| | | CTGAGCAACCATGGAATGTCCTGGGTCAGAAGGGCCCCTGGAAAAGGCC |
| | | TCGAATGGGTGTCAGGGATCGTGTACTCCGGTTCCACTTACTACGCCGC |
| | | CTCCGTGAAGGGGCGCTTCACTATCTCACGGGATAACTCCCGCAATACC |
| | | CTGTACCTCCAAATGAACAGCCTGCGGCCGGAGGATACCGCCATCTACT |
| | | ACTGTTCCGCCCACGGTGGAGAGTCTGACGTCTGGGGCCAGGGAACTAC |
| | | CGTGACCGTGTCCTCCGCGTCCGGCGGTGGAGGGAGCGGCGGCCGCGCC |
| | | AGCGGCGGCGGAGGCTCCGAGATCGTGATGACCCAGAGCCCCGCTACTC |
| | | TGTCGGTGTCGCCCGGAGAAAGGGCGACCCTGTCCTGCCGGGCGTCGCA |
| | | GTCCGTGAGCAGCAAGCTGGCTTGGTACCAGCAGAAGCCGGGCCAGGCA |
| | | CCACGCCTGCTTATGTACGGTGCCTCCATTCGGGCCACCGGAATCCCGG |
| | | ACCGGTTCTCGGGGTCGGGGTCCGGTACCGAGTTCACACTGACCATTTC |
| | | CTCGCTCGAGCCCGAGGACTTTGCCGTCTATTACTGCCAGCAGTACGGC |
| | | TCCTCCTCATGGACGTTCGGCCAGGGGACCAAGGTCGAAATCAAGACCA |
| | | CTACCCCAGCACCGAGGCCACCCACCCCGGCTCCTACCATCGCCTCCCA |
| | | GCCTCTGTCCCTGCGTCCGGAGGCATGTAGACCCGCAGCTGGTGGGGCC |
| | | GTGCATACCCGGGGTCTTGACTTCGCCTGCGATATCTACATTTGGGCCC |
| | | CTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCC |
| | | GGTTCCCAGAGGAGGAGGAAGGCGGCTGCGAACTGCGCGTGAAATTCAG |
| | | CCGCAGCGCAGATGCTCCAGCCTACAAGCAGGGGCAGAACCAGCTCTAC |
| | | AACGAACTCAATCTTGGTCGGAGAGAGGAGTACGACGTGCTGGACAAGC |
| | | GGAGAGGACGGGACCCAGAAATGGGCGGGAAGCCGCGCAGAAAGAATCC |
| | | CCAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCACG |
| | | ACGGACTGTACCAGGGACTCAGCACCGCCACCAAGGACACCTATGACGC |
| | | TCTTCACATGCAGGCCCTGCCGCCTCGG |

139107

| | | |
|---|---|---|
| 139107-aa | 47 | EVQLVETGGGVVQPGGSLRLSCAVSGFALSNHGMSWVRRAPGKGLEWVS |
| ScFv domain | | GIVYSGSTYYAASVKGRFTISRDNSRNTLYLQMNSLRPEDTAIYYCSAH |
| | | GGESDVWGQGTTVTVSSASGGGGSGGRASGGGGSEIVLTQSPGTLSLSP |
| | | GERATLSCRASQSVGSTNLAWYQQKPGQAPRLLIYDASNRATGIPDRFS |
| | | GGGSGTDFTLTISRLEPEDFAVYYCQQYGSSPPWTFGQGTKVEIK |
| 139107-nt | 62 | GAAGTGCAATTGGTGGAGACTGGAGGAGGAGTGGTGCAACCTGGAGGAA |
| ScFv domain | | GCCTGAGACTGTCATGCGCGGTGTCGGGCTTCGCCCTCTCCAACCACGG |
| | | AATGTCCTGGGTCCGCCGGGCCCCTGGGAAAGGACTTGAATGGGTGTCC |
| | | GGCATCGTGTACTCGGGTTCCACCTACTACGCGGCCTCAGTGAAGGGCC |
| | | GGTTTACTATTAGCCGCGACAACTCCAGAAACACACTGTACCTCCAAAT |
| | | GAACTCGCTGCGGCCGGAAGATACCGCTATCTACTACTGCTCCGCCCAT |
| | | GGGGGAGAGTCGGACGTCTGGGGACAGGGCACCACTGTCACTGTGTCCA |
| | | GCGCTTCCGGCGGTGGTGGAAGCGGGGGACGGGCCTCAGGAGGCGGTGG |
| | | CAGCGAGATTGTGCTGACCCAGTCCCCCGGGACCCTGAGCCTGTCCCCG |
| | | GGAGAAAGGGCCACCCTCTCCTGTCGGGCATCCCAGTCCGTGGGGTCTA |
| | | CTAACCTTGCATGGTACCAGCAGAAGCCCGGCCAGGCCCCTCGCCTGCT |
| | | GATCTACGACGCGTCCAATAGAGCCACCGGCATCCCGGATCGCTTCAGC |
| | | GGAGGCGGATCGGGCACCGACTTCACCCTCACCATTTCAAGGCTGGAAC |
| | | CGGAGGACTTCGCCGTGTACTACTGCCAGCAGTATGGTTCGTCCCCACC |
| | | CTGGACGTTCGGCCAGGGGACTAAGGTCGAGATCAAG |
| 139107-aa | 77 | EVQLVETGGGVVQPGGSLRLSCAVSGFALSNHGMSWVRRAPGKGLEWVS |
| VH | | GIVYSGSTYYAASVKGRFTISRDNSRNTLYLQMNSLRPEDTAIYYCSAH |
| | | GGESDVWGQGTTVTVSS |
| 139107-aa | 92 | EIVLTQSPGTLSLSPGERATLSCRASQSVGSTNLAWYQQKPGQAPRLLI |
| VL | | YDASNRATGIPDRFSGGGSGTDFTLTISRLEPEDFAVYYCQQYGSSPPW |
| | | TFGQGTKVEIK |
| 139107-aa | 107 | MALPVTALLLPLALLLHAARPEVQLVETGGGVVQPGGSLRLSCAVSGFA |
| Full CAR | | LSNHGMSWVRRAPGKGLEWVSGIVYSGSTYYAASVKGRFTISRDNSRNT |
| | | LYLQMNSLRPEDTAIYYCSAHGGESDVWGQGTTVTVSSASGGGGSGGRA |
| | | SGGGGSEIVLTQSPGTLSLSPGERATLSCRASQSVGSTNLAWYQQKPGQ |
| | | APRLLIYDASNRATGIPDRFSGGGSGTDFTLTISRLEPEDFAVYYCQQY |
| | | GSSPPWTFGQGTKVEIKTTTPAPRPPTPAPTIASQPLSLRPEACRPAAG |
| | | GAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQ |
| | | LYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 139107-nt | 122 | ATGGCCCTCCCTGTCACCGCCCTGCTGCTTCCGCTGGCTCTTCTGCTCC |
| Full CAR | | ACGCCGCTCGGCCCGAAGTGCAATTGGTGGAGACTGGAGGAGGAGTGGT |
| | | GCAACCTGGAGGAAGCCTGAGACTGTCATGCGCGGTGTCGGGCTTCGCC |
| | | CTCTCCAACCACGGAATGTCCTGGGTCCGCCGGGCCCCTGGGAAAGGAC |
| | | TTGAATGGGTGTCCGGCATCGTGTACTCGGGTTCCACCTACTACGCGGC |
| | | CTCAGTGAAGGGCCGGTTTACTATTAGCCGCGACAACTCCAGAAACACA |
| | | CTGTACCTCCAAATGAACTCGCTGCGGCCGGAAGATACCGCTATCTACT |
| | | ACTGCTCCGCCCATGGGGGAGAGTCGGACGTCTGGGGACAGGGCACCAC |
| | | TGTCACTGTGTCCAGCGCTTCCGGCGGTGGTGGAAGCGGGGGACGGGCC |
| | | TCAGGAGGCGGTGGCAGCGAGATTGTGCTGACCCAGTCCCCCGGGACCC |
| | | TGAGCCTGTCCCCGGGAGAAAGGGCCACCCTCTCCTGTCGGGCATCCCA |
| | | GTCCGTGGGGTCTACTAACCTTGCATGGTACCAGCAGAAGCCCGGCCAG |
| | | GCCCCTCGCCTGCTGATCTACGACGCGTCCAATAGAGCCACCGGCATCC |
| | | CGGATCGCTTCAGCGGAGGCGGATCGGGCACCGACTTCACCCTCACCAT |
| | | TTCAAGGCTGGAACCGGAGGACTTCGCCGTGTACTACTGCCAGCAGTAT |
| | | GGTTCGTCCCCACCCTGGACGTTCGGCCAGGGGACTAAGGTCGAGATCA |
| | | AGACCACTACCCCAGCACCGAGGCCACCCACCCCGGCTCCTACCATCGC |
| | | CTCCCAGCCTCTGTCCCTGCGTCCGGAGGCATGTAGACCCGCAGCTGGT |
| | | GGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTT |
| | | CATGCCGGTTCCCAGAGGAGGAGGAAGGCGGCTGCGAACTGCGCGTGAA |
| | | ATTCAGCCGCAGCGCAGATGCTCCAGCCTACAAGCAGGGGCAGAACCAG |
| | | CTCTACAACGAACTCAATCTTGGTCGGAGAGAGGAGTACGACGTGCTGG |
| | | ACAAGCGGAGAGGACGGGACCCAGAAATGGGCGGGAAGCCGCGCAGAAA |
| | | GAATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAAG |
| | | GCCACGACGGACTGTACCAGGGACTCAGCACCGCCACCAAGGACACCTA |
| | | TGACGCTCTTCACATGCAGGCCCTGCCGCCTCGG |

139108

| | | |
|---|---|---|
| 139108-aa | 48 | QVQLVESGGGLVKPGGSLRLSCAASGFTFSDYYMSWIRQAPGKGLEWVS |
| ScFv domain | | YISSSGSTIYYADSVKGRFTISRDNAKNSLYLQMNSLRAEDTAVYYCAR |
| | | ESGDGMDVWGQGTTVTVSSASGGGGSGGRASGGGGSDIQMTQSPSSLSA |
| | | SVGDRVTITCRASQSISSYLNWYQQKPGKAPKLLIYAASSLQSGVPSRF |
| | | SGSGSGTDFTLTISSLQPEDFATYYCQQSYTLAFGQGTKVDIK |
| 139108-nt | 63 | CAAGTGCAACTCGTGGAATCTGGTGGAGGACTCGTGAAACCTGGAGGAT |
| ScFv domain | | CATTGAGACTGTCATGCGCGGCCTCGGGATTCACGTTCTCCGATTACTA |
| | | CATGAGCTGGATTCGCCAGGCTCCGGGGAAGGGACTGGAATGGGTGTCC |
| | | TACATTTCCTCATCCGGCTCCACCATCTACTACGCGGACTCCGTGAAGG |
| | | GGAGATTCACCATTAGCCGCGATAACGCCAAGAACAGCCTGTACCTTCA |
| | | GATGAACTCCCTGCGGGCTGAAGATACTGCCGTCTACTACTGCGCAAGG |
| | | GAGAGCGGAGATGGGATGGACGTCTGGGGACAGGGTACCACTGTGACCG |
| | | TGTCGTCGGCCTCCGGCGGAGGGGGTTCGGGTGGAAGGGCCAGCGGCGG |
| | | CGGAGGCAGCGACATCCAGATGACCCAGTCCCCCTCATCGCTGTCCGCC |
| | | TCCGTGGGCGACCGCGTCACCATCACATGCCGGGCCTCACAGTCGATCT |
| | | CCTCCTACCTCAATTGGTATCAGCAGAAGCCCGGAAAGGCCCCTAAGCT |
| | | TCTGATCTACGCAGCGTCCTCCCTGCAATCCGGGGTCCCATCTCGGTTC |
| | | TCCGGCTCGGGCAGCGGTACCGACTTCACTCTGACCATCTCGAGCCTGC |
| | | AGCCGGAGGACTTCGCCACTTACTACTGTCAGCAAAGCTACACCCTCGC |
| | | GTTTGGCCAGGGCACCAAAGTGGACATCAAG |
| 139108-aa | 78 | QVQLVESGGGLVKPGGSLRLSCAASGFTFSDYYMSWIRQAPGKGLEWVS |
| VH | | YISSSGSTIYYADSVKGRFTISRDNAKNSLYLQMNSLRAEDTAVYYCAR |
| | | ESGDGMDVWGQGTTVTVSS |
| 139108-aa | 93 | DIQMTQSPSSLSASVGDRVTITCRASQSISSYLNWYQQKPGKAPKLLIY |
| VL | | AASSLQSGVPSRFSGSGSGTDFTLTISSLQPEDFATYYCQQSYTLAFGQ |
| | | GTKVDIK |
| 139108-aa | 108 | MALPVTALLLPLALLLHAARPQVQLVESGGGLVKPGGSLRLSCAASGFT |
| Full CAR | | FSDYYMSWIRQAPGKGLEWVSYISSSGSTIYYADSVKGRFTISRDNAKN |
| | | SLYLQMNSLRAEDTAVYYCARESGDGMDVWGQGTTVTVSSASGGGGSGG |
| | | RASGGGGSDIQMTQSPSSLSASVGDRVTITCRASQSISSYLNWYQQKPG |
| | | KAPKLLIYAASSLQSGVPSRFSGSGSGTDFTLTISSLQPEDFATYYCQQ |
| | | SYTLAFGQGTKVDIKTTTPAPRPPTPAPTIASQPLSLRPEACRPAAGGA |
| | | VHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 139108-nt | 123 | ATGGCCCTCCCTGTCACCGCCCTGCTGCTTCCGCTGGCTCTTCTGCTCC |
| Full CAR | | ACGCCGCTCGGCCCCAAGTGCAACTCGTGGAATCTGGTGGAGGACTCGT |
| | | GAAACCTGGAGGATCATTGAGACTGTCATGCGCGGCCTCGGGATTCACG |
| | | TTCTCCGATTACTACATGAGCTGGATTCGCCAGGCTCCGGGGAAGGGAC |
| | | TGGAATGGGTGTCCTACATTTCCTCATCCGGCTCCACCATCTACTACGC |
| | | GGACTCCGTGAAGGGGAGATTCACCATTAGCCGCGATAACGCCAAGAAC |
| | | AGCCTGTACCTTCAGATGAACTCCCTGCGGGCTGAAGATACTGCCGTCT |
| | | ACTACTGCGCAAGGGAGAGCGGAGATGGGATGGACGTCTGGGGACAGGG |
| | | TACCACTGTGACCGTGTCGTCGGCCTCCGGCGGAGGGGGTTCGGGTGGA |
| | | AGGGCCAGCGGCGGCGGAGGCAGCGACATCCAGATGACCCAGTCCCCCT |
| | | CATCGCTGTCCGCCTCCGTGGGCGACCGCGTCACCATCACATGCCGGGC |
| | | CTCACAGTCGATCTCCTCCTACCTCAATTGGTATCAGCAGAAGCCCGGA |
| | | AAGGCCCCTAAGCTTCTGATCTACGCAGCGTCCTCCCTGCAATCCGGGG |
| | | TCCCATCTCGGTTCTCCGGCTCGGGCAGCGGTACCGACTTCACTCTGAC |
| | | CATCTCGAGCCTGCAGCCGGAGGACTTCGCCACTTACTACTGTCAGCAA |
| | | AGCTACACCCTCGCGTTTGGCCAGGGCACCAAAGTGGACATCAAGACCA |
| | | CTACCCCAGCACCGAGGCCACCCACCCCGGCTCCTACCATCGCCTCCCA |
| | | GCCTCTGTCCCTGCGTCCGGAGGCATGTAGACCCGCAGCTGGTGGGGCC |
| | | GTGCATACCCGGGGTCTTGACTTCGCCTGCGATATCTACATTTGGGCCC |
| | | CTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCC |
| | | GGTTCCCAGAGGAGGAGGAAGGCGGCTGCGAACTGCGCGTGAAATTCAG |
| | | CCGCAGCGCAGATGCTCCAGCCTACAAGCAGGGGCAGAACCAGCTCTAC |
| | | AACGAACTCAATCTTGGTCGGAGAGAGGAGTACGACGTGCTGGACAAGC |
| | | GGAGAGGACGGGACCCAGAAATGGGCGGGAAGCCGCGCAGAAAGAATCC |
| | | CCAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCACG |
| | | ACGGACTGTACCAGGGACTCAGCACCGCCACCAAGGACACCTATGACGC |
| | | TCTTCACATGCAGGCCCTGCCGCCTCGG |

139110

| | | |
|---|---|---|
| 139110-aa | 50 | QVQLVQSGGGLVKPGGSLRLSCAASGFTFSDYYMSWIRQAPGKGLEWVS |
| ScFv domain | | YISSSGNTIYYADSVKGRFTISRDNAKNSLYLQMNSLRAEDTAVYYCAR |
| | | STMVREDYWGQGTLVTVSSASGGGGSGGRASGGGGSDIVLTQSPLSLPV |
| | | TLGQPASISCKSSESLVHNSGKTYLNWFHQRPGQSPRRLIYEVSNRDSG |
| | | VPDRFTGSGSGTDFTLKISRVEAEDVGVYYCMQGTHWPGTFGQGTKLEI |
| | | K |
| 139110-nt | 65 | CAAGTGCAACTGGTGCAAAGCGGAGGAGGATTGGTCAAACCCGGAGGAA |
| ScFv domain | | GCCTGAGACTGTCATGCGCGGCCTCTGGATTCACCTTCTCCGATTACTA |
| | | CATGTCATGGATCAGACAGGCCCCGGGGAAGGGCCTCGAATGGGTGTCC |
| | | TACATCTCGTCCTCCGGGAACACCATCTACTACGCCGACAGCGTGAAGG |
| | | GCCGCTTTACCATTTCCCGCGACAACGCAAAGAACTCGCTGTACCTTCA |
| | | GATGAATTCCCTGCGGGCTGAAGATACCGCGGTGTACTATTGCGCCCGG |
| | | TCCACTATGGTCCGGGAGGACTACTGGGGACAGGGCACACTCGTGACCG |
| | | TGTCCAGCGCGAGCGGGGGTGGAGGCAGCGGTGGACGCGCCTCCGGCGG |
| | | CGGCGGTTCAGACATCGTGCTGACTCAGTCGCCCCTGTCGCTGCCGGTC |
| | | ACCCTGGGCCAACCGGCCTCAATTAGCTGCAAGTCCTCGGAGAGCCTGG |
| | | TGCACAACTCAGGAAAGACTTACCTGAACTGGTTCCATCAGCGGCCTGG |
| | | ACAGTCCCCACGGAGGCTCATCTATGAAGTGTCCAACAGGGATTCGGGG |
| | | GTGCCCGACCGCTTCACTGGCTCCGGGTCCGGCACCGACTTCACCTTGA |
| | | AAATCTCCAGAGTGGAAGCCGAGGACGTGGGCGTGTACTACTGTATGCA |
| | | GGGTACCCACTGGCCTGGAACCTTTGGACAAGGAACTAAGCTCGAGATT |
| | | AAG |
| 139110-aa | 80 | QVQLVQSGGGLVKPGGSLRLSCAASGFTFSDYYMSWIRQAPGKGLEWVS |
| VH | | YISSSGNTIYYADSVKGRFTISRDNAKNSLYLQMNSLRAEDTAVYYCAR |
| | | STMVREDYWGQGTLVTVSS |
| 139110-aa | 95 | DIVLTQSPLSLPVTLGQPASISCKSSESLVHNSGKTYLNWFHQRPGQSP |
| VL | | RRLIYEVSNRDSGVPDRFTGSGSGTDFTLKISRVEAEDVGVYYCMQGTH |
| | | WPGTFGQGTKLEIK |
| 139110-aa | 110 | MALPVTALLLPLALLLHAARPQVQLVQSGGGLVKPGGSLRLSCAASGFT |
| Full CAR | | FSDYYMSWIRQAPGKGLEWVSYISSSGNTIYYADSVKGRFTISRDNAKN |
| | | SLYLQMNSLRAEDTAVYYCARSTMVREDYWGQGTLVTVSSASGGGGSGG |
| | | RASGGGGSDIVLTQSPLSLPVTLGQPASISCKSSESLVHNSGKTYLNWF |
| | | HQRPGQSPRRLIYEVSNRDSGVPDRFTGSGSGTDFTLKISRVEAEDVGV |
| | | YYCMQGTHWPGTFGQGTKLEIKTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 139110-nt | 125 | ATGGCCCTCCCTGTCACCGCCCTGCTGCTTCCGCTGGCTCTTCTGCTCC |
| Full CAR | | ACGCCGCTCGGCCCCAAGTGCAACTGGTGCAAAGCGGAGGAGGATTGGT |
| | | CAAACCCGGAGGAAGCCTGAGACTGTCATGCGCGGCCTCTGGATTCACC |
| | | TTCTCCGATTACTACATGTCATGGATCAGACAGGCCCCGGGGAAGGGCC |
| | | TCGAATGGGTGTCCTACATCTCGTCCTCCGGGAACACCATCTACTACGC |
| | | CGACAGCGTGAAGGGCCGCTTTACCATTTCCCGCGACAACGCAAAGAAC |
| | | TCGCTGTACCTTCAGATGAATTCCCTGCGGGCTGAAGATACCGCGGTGT |
| | | ACTATTGCGCCCGGTCCACTATGGTCCGGGAGGACTACTGGGGACAGGG |
| | | CACACTCGTGACCGTGTCCAGCGCGAGCGGGGGTGGAGGCAGCGGTGGA |
| | | CGCGCCTCCGGCGGCGGCGGTTCAGACATCGTGCTGACTCAGTCGCCCC |
| | | TGTCGCTGCCGGTCACCCTGGGCCAACCGGCCTCAATTAGCTGCAAGTC |
| | | CTCGGAGAGCCTGGTGCACAACTCAGGAAAGACTTACCTGAACTGGTTC |
| | | CATCAGCGGCCTGGACAGTCCCCACGGAGGCTCATCTATGAAGTGTCCA |
| | | ACAGGGATTCGGGGGTGCCCGACCGCTTCACTGGCTCCGGGTCCGGCAC |
| | | CGACTTCACCTTGAAAATCTCCAGAGTGGAAGCCGAGGACGTGGGCGTG |
| | | TACTACTGTATGCAGGGTACCCACTGGCCTGGAACCTTTGGACAAGGAA |
| | | CTAAGCTCGAGATTAAGACCACTACCCCAGCACCGAGGCCACCCACCCC |
| | | GGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAG |
| | | AGGAGGACGGCTGTTCATGCCGGTTCCCAGAGGAGGAGGAAGGCGGCTG |
| | | CGAACTGCGCGTGAAATTCAGCCGCAGCGCAGATGCTCCAGCCTACAAG |
| | | CAGGGGCAGAACCAGCTCTACAACGAACTCAATCTTGGTCGGAGAGAGG |
| | | AGTACGACGTGCTGGACAAGCGGAGAGGACGGGACCCAGAAATGGGCGG |
| | | GAAGCCGCGCAGAAAGAATCCCCAAGAGGGCCTGTACAACGAGCTCCAA |
| | | AAGGATAAGATGGCAGAAGCCTATAGCGAGATTGGTATGAAAGGGGAAC |
| | | GCAGAAGAGGCAAAGGCCACGACGGACTGTACCAGGGACTCAGCACCGC |
| | | CACCAAGGACACCTATGACGCTCTTCACATGCAGGCCCTGCCGCCTCGG |

139112

| | | |
|---|---|---|
| 139112-aa | 51 | QVQLVESGGGLVQPGGSLRLSCAVSGFALSNHGMSWVRRAPGKGLEWVS |
| ScFv domain | | GIVYSGSTYYAASVKGRFTISRDNSRNTLYLQMNSLRPEDTAIYYCSAH |
| | | GGESDVWGQGTTVTVSSASGGGGSGGRASGGGGSDIRLTQSPSPLSASV |
| | | GDRVTITCQASEDINKFLNWYHQTPGKAPKLLIYDASTLQTGVPSRFSG |
| | | SGSGTDFTLTINSLQPEDIGTYYCQQYESLPLTFGGGTKVEIK |
| 139112-nt | 66 | CAAGTGCAACTCGTGGAATCTGGTGGAGGACTCGTGCAACCCGGTGGAA |
| ScFv domain | | GCCTTAGGCTGTCGTGCGCCGTCAGCGGGTTTGCTCTGAGCAACCATGG |
| | | AATGTCCTGGGTCCGCCGGGCACCGGGAAAAGGGCTGGAATGGGTGTCC |
| | | GGCATCGTGTACAGCGGGTCAACCTATTACGCCGCGTCCGTGAAGGGCA |
| | | GATTCACTATCTCAAGAGACAACAGCCGGAACACCCTGTACTTGCAAAT |
| | | GAATTCCCTGCGCCCCGAGGACACCGCCATCTACTACTGCTCCGCCCAC |
| | | GGAGGAGAGTCGGACGTGTGGGGCCAGGGAACGACTGTGACTGTGTCCA |
| | | GCGCATCAGGAGGGGGTGGTTCGGGCGGCCGGGCCTCGGGGGGAGGAGG |
| | | TTCCGACATTCGGCTGACCCAGTCCCCGTCCCCACTGTCGGCCTCCGTC |
| | | GGCGACCGCGTGACCATCACTTGTCAGGCGTCCGAGGACATTAACAAGT |
| | | TCCTGAACTGGTACCACCAGACCCCTGGAAAGGCCCCCAAGCTGCTGAT |
| | | CTACGATGCCTCGACCCTTCAAACTGGAGTGCCTAGCCGGTTCTCCGGG |
| | | TCCGGCTCCGGCACTGATTTCACTCTGACCATCAACTCATTGCAGCCGG |
| | | AAGATATCGGGACCTACTATTGCCAGCAGTACGAATCCCTCCCGCTCAC |
| | | ATTCGGCGGGGGAACCAAGGTCGAGATTAAG |
| 139112-aa | 81 | QVQLVESGGGLVQPGGSLRLSCAVSGFALSNHGMSWVRRAPGKGLEWVS |
| VH | | GIVYSGSTYYAASVKGRFTISRDNSRNTLYLQMNSLRPEDTAIYYCSAH |
| | | GGESDVWGQGTTVTVSS |
| 139112-aa | 96 | DIRLTQSPSPLSASVGDRVTITCQASEDINKFLNWYHQTPGKAPKLLIY |
| VL | | DASTLQTGVPSRFSGSGSGTDFTLTINSLQPEDIGTYYCQQYESLPLTF |
| | | GGGTKVEIK |
| 139112-aa | 111 | MALPVTALLLPLALLLHAARPQVQLVESGGGLVQPGGSLRLSCAVSGFA |
| Full CAR | | LSNHGMSWVRRAPGKGLEWVSGIVYSGSTYYAASVKGRFTISRDNSRNT |
| | | LYLQMNSLRPEDTAIYYCSAHGGESDVWGQGTTVTVSSASGGGGSGGRA |
| | | SGGGGSDIRLTQSPSPLSASVGDRVTITCQASEDINKFLNWYHQTPGKA |
| | | PKLLIYDASTLQTGVPSRFSGSGSGTDFTLTINSLQPEDIGTYYCQQYE |
| | | SLPLTFGGGTKVEIKTTTPAPRPPTPAPTIASQPLSLRPEACRPAAGGA |
| | | VHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 139112-nt | 126 | ATGGCCCTCCCTGTCACCGCCCTGCTGCTTCCGCTGGCTCTTCTGCTCC |
| Full CAR | | ACGCCGCTCGGCCCCAAGTGCAACTCGTGGAATCTGGTGGAGGACTCGT |
| | | GCAACCCGGTGGAAGCCTTAGGCTGTCGTGCGCCGTCAGCGGGTTTGCT |
| | | CTGAGCAACCATGGAATGTCCTGGGTCCGCCGGGCACCGGGAAAAGGGC |
| | | TGGAATGGGTGTCCGGCATCGTGTACAGCGGGTCAACCTATTACGCCGC |
| | | GTCCGTGAAGGGCAGATTCACTATCTCAAGAGACAACAGCCGGAACACC |
| | | CTGTACTTGCAAATGAATTCCCTGCGCCCCGAGGACACCGCCATCTACT |
| | | ACTGCTCCGCCCACGGAGGAGAGTCGGACGTGTGGGGCCAGGGAACGAC |
| | | TGTGACTGTGTCCAGCGCATCAGGAGGGGGTGGTTCGGGCGGCCGGGCC |
| | | TCGGGGGGAGGAGGTTCCGACATTCGGCTGACCCAGTCCCCGTCCCCAC |
| | | TGTCGGCCTCCGTCGGCGACCGCGTGACCATCACTTGTCAGGCGTCCGA |
| | | GGACATTAACAAGTTCCTGAACTGGTACCACCAGACCCCTGGAAAGGCC |
| | | CCCAAGCTGCTGATCTACGATGCCTCGACCCTTCAAACTGGAGTGCCTA |
| | | GCCGGTTCTCCGGGTCCGGCTCCGGCACTGATTTCACTCTGACCATCAA |
| | | CTCATTGCAGCCGGAAGATATCGGGACCTACTATTGCCAGCAGTACGAA |
| | | TCCCTCCCGCTCACATTCGGCGGGGGAACCAAGGTCGAGATTAAGACCA |
| | | CTACCCCAGCACCGAGGCCACCCACCCCGGCTCCTACCATCGCCTCCCA |
| | | GCCTCTGTCCCTGCGTCCGGAGGCATGTAGACCCGCAGCTGGTGGGGCC |
| | | GTGCATACCCGGGGTCTTGACTTCGCCTGCGATATCTACATTTGGGCCC |
| | | CTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCC |
| | | GGTTCCCAGAGGAGGAGGAAGGCGGCTGCGAACTGCGCGTGAAATTCAG |
| | | CCGCAGCGCAGATGCTCCAGCCTACAAGCAGGGGCAGAACCAGCTCTAC |
| | | AACGAACTCAATCTTGGTCGGAGAGAGGAGTACGACGTGCTGGACAAGC |
| | | GGAGAGGACGGGACCCAGAAATGGGCGGGAAGCCGCGCAGAAAGAATCC |
| | | CCAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCACG |
| | | ACGGACTGTACCAGGGACTCAGCACCGCCACCAAGGACACCTATGACGC |
| | | TCTTCACATGCAGGCCCTGCCGCCTCGG |

139113

| | | |
|---|---|---|
| 139113-aa | 52 | EVQLVETGGGLVQPGGSLRLSCAVSGFALSNHGMSWVRRAPGKGLEWVS |
| ScFv domain | | GIVYSGSTYYAASVKGRFTISRDNSRNTLYLQMNSLRPEDTAIYYCSAH |
| | | GGESDVWGQGTTVTVSSASGGGGSGGRASGGGGSETTLTQSPATLSVSP |
| | | GERATLSCRASQSVGSNLAWYQQKPGQGPRLLIYGASTRATGIPARFSG |
| | | SGSGTEFTLTISSLQPEDFAVYYCQQYNDWLPVTFGQGTKVEIK |
| 139113-nt | 67 | GAAGTGCAATTGGTGGAAACTGGAGGAGGACTTGTGCAACCTGGAGGAT |
| ScFv domain | | CATTGCGGCTCTCATGCGCTGTCTCCGGCTTCGCCCTGTCAAATCACGG |
| | | GATGTCGTGGGTCAGACGGGCCCCGGGAAAGGGTCTGGAATGGGTGTCG |
| | | GGGATTGTGTACAGCGGCTCCACCTACTACGCCGCTTCGGTCAAGGGCC |
| | | GCTTCACTATTTCACGGGACAACAGCCGCAACACCCTCTATCTGCAAAT |
| | | GAACTCTCTCCGCCCGGAGGATACCGCCATCTACTACTGCTCCGCACAC |
| | | GGCGGCGAATCCGACGTGTGGGGACAGGGAACCACTGTCACCGTGTCGT |
| | | CCGCATCCGGTGGCGGAGGATCGGGTGGCCGGGCCTCCGGGGGCGGCGG |
| | | CAGCGAGACTACCCTGACCCAGTCCCCTGCCACTCTGTCCGTGAGCCCG |
| | | GGAGAGAGAGCCACCCTTAGCTGCCGGGCCAGCCAGAGCGTGGGCTCCA |
| | | ACCTGGCCTGGTACCAGCAGAAGCCAGGACAGGGTCCCAGGCTGCTGAT |
| | | CTACGGAGCCTCCACTCGCGCGACCGGCATCCCCGCGAGGTTCTCCGGG |
| | | TCGGGTTCCGGGACCGAGTTCACCCTGACCATCTCCTCCCTCCAACCGG |
| | | AGGACTTCGCGGTGTACTACTGTCAGCAGTACAACGATTGGCTGCCCGT |
| | | GACATTTGGACAGGGGACGAAGGTGGAAATCAAA |
| 139113-aa | 82 | EVQLVETGGGLVQPGGSLRLSCAVSGFALSNHGMSWVRRAPGKGLEWVS |
| VH | | GIVYSGSTYYAASVKGRFTISRDNSRNTLYLQMNSLRPEDTAIYYCSAH |
| | | GGESDVWGQGTTVTVSS |
| 139113-aa | 97 | ETTLTQSPATLSVSPGERATLSCRASQSVGSNLAWYQQKPGQGPRLLIY |
| VL | | GASTRATGIPARFSGSGSGTEFTLTISSLQPEDFAVYYCQQYNDWLPVT |
| | | FGQGTKVEIK |
| 139113-aa | 112 | MALPVTALLLPLALLLHAARPEVQLVETGGGLVQPGGSLRLSCAVSGFA |
| Full CAR | | LSNHGMSWVRRAPGKGLEWVSGIVYSGSTYYAASVKGRFTISRDNSRNT |
| | | LYLQMNSLRPEDTAIYYCSAHGGESDVWGQGTTVTVSSASGGGGSGGRA |
| | | SGGGGSETTLTQSPATLSVSPGERATLSCRASQSVGSNLAWYQQKPGQG |
| | | PRLLIYGASTRATGIPARFSGSGSGTEFTLTISSLQPEDFAVYYCQQYN |
| | | DWLPVTFGQGTKVEIKTTTPAPRPPTPAPTIASQPLSLRPEACRPAAGG |
| | | AVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQL |
| | | YNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 139113-nt | 127 | ATGGCCCTCCCTGTCACCGCCCTGCTGCTTCCGCTGGCTCTTCTGCTCC |
| Full CAR | | ACGCCGCTCGGCCCGAAGTGCAATTGGTGGAAACTGGAGGAGGACTTGT |
| | | GCAACCTGGAGGATCATTGCGGCTCTCATGCGCTGTCTCCGGCTTCGCC |
| | | CTGTCAAATCACGGGATGTCGTGGGTCAGACGGGCCCCGGGAAAGGGTC |
| | | TGGAATGGGTGTCGGGGATTGTGTACAGCGGCTCCACCTACTACGCCGC |
| | | TTCGGTCAAGGGCCGCTTCACTATTTCACGGGACAACAGCCGCAACACC |
| | | CTCTATCTGCAAATGAACTCTCTCCGCCCGGAGGATACCGCCATCTACT |
| | | ACTGCTCCGCACACGGCGGCGAATCCGACGTGTGGGGACAGGGAACCAC |
| | | TGTCACCGTGTCGTCCGCATCCGGTGGCGGAGGATCGGGTGGCCGGGCC |
| | | TCCGGGGGCGGCGGCAGCGAGACTACCCTGACCCAGTCCCCTGCCACTC |
| | | TGTCCGTGAGCCCGGGAGAGAGAGCCACCCTTAGCTGCCGGGCCAGCCA |
| | | GAGCGTGGGCTCCAACCTGGCCTGGTACCAGCAGAAGCCAGGACAGGGT |
| | | CCCAGGCTGCTGATCTACGGAGCCTCCACTCGCGCGACCGGCATCCCCG |
| | | CGAGGTTCTCCGGGTCGGGTTCCGGGACCGAGTTCACCCTGACCATCTC |
| | | CTCCCTCCAACCGGAGGACTTCGCGGTGTACTACTGTCAGCAGTACAAC |
| | | GATTGGCTGCCCGTGACATTTGGACAGGGGACGAAGGTGGAAATCAAAA |
| | | CCACTACCCCAGCACCGAGGCCACCCACCCCGGCTCCTACCATCGCCTC |
| | | CCAGCCTCTGTCCCTGCGTCCGGAGGCATGTAGACCCGCAGCTGGTGGG |
| | | GCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCAC |
| | | TCTTTACTGTAAGCGCGGTCGGAAGAAGCTGCTGTACATCTTTAAGCAA |
| | | CCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATT |
| | | CAGCCGCAGCGCAGATGCTCCAGCCTACAAGCAGGGGCAGAACCAGCTC |
| | | TACAACGAACTCAATCTTGGTCGGAGAGAGGAGTACGACGTGCTGGACA |
| | | AGCGGAGAGGACGGGACCCAGAAATGGGCGGGAAGCCGCGCAGAAAGAA |
| | | TCCCCAAGAGGGCCTGTACAACGAGCTCCAAAAGGATAAGATGGCAGAA |
| | | GCCTATAGCGAGATTGGTATGAAAGGGGAACGCAGAAGAGGCAAAGGCC |
| | | ACGACGGACTGTACCAGGGACTCAGCACCGCCACCAAGGACACCTATGA |
| | | CGCTCTTCACATGCAGGCCCTGCCGCCTCGG |

139114

| | | |
|---|---|---|
| 139114-aa | 53 | EVQLVESGGGLVQPGGSLRLSCAVSGFALSNHGMSWVRRAPGKGLEWVS |
| ScFv domain | | GIVYSGSTYYAASVKGRFTISRDNSRNTLYLQMNSLRPEDTAIYYCSAH |
| | | GGESDVWGQGTTVTVSSASGGGGSGGRASGGGGSEIVLTQSPGTLSLSP |
| | | GERATLSCRASQSIGSSSLAWYQQKPGQAPRLLMYGASSRASGIPDRFS |
| | | GSGSGTDFTLTISRLEPEDFAVYYCQQYAGSPPFTFGQGTKVEIK |
| 139114-nt | 68 | GAAGTGCAATTGGTGGAATCTGGTGGAGGACTTGTGCAACCTGGAGGAT |
| ScFv domain | | CACTGAGACTGTCATGCGCGGTGTCCGGTTTTGCCCTGAGCAATCATGG |
| | | GATGTCGTGGGTCCGGCGCGCCCCCGGAAAGGGTCTGGAATGGGTGTCG |
| | | GGTATCGTCTACTCCGGGAGCACTTACTACGCCGCGAGCGTGAAGGGCC |
| | | GCTTCACCATTTCCCGCGATAACTCCCGCAACACCCTGTACTTGCAAAT |
| | | GAACTCGCTCCGGCCTGAGGACACTGCCATCTACTACTGCTCCGCACAC |
| | | GGAGGAGAATCCGACGTGTGGGGCCAGGGAACTACCGTGACCGTCAGCA |
| | | GCGCCTCCGGCGGCGGGGGCTCAGGCGGACGGGCTAGCGGCGGCGGTGG |
| | | CTCCGAGATCGTGCTGACCCAGTCGCCTGGCACTCTCTCGCTGAGCCCC |
| | | GGGGAAAGGGCAACCCTGTCCTGTCGGGCCAGCCAGTCCATTGGATCAT |
| | | CCTCCCTCGCCTGGTATCAGCAGAAACCGGGACAGGCTCCGCGGCTGCT |
| | | TATGTATGGGGCCAGCTCAAGAGCCTCCGGCATTCCCGACCGGTTCTCC |
| | | GGGTCCGGTTCCGGCACCGATTTCACCCTGACTATCTCGAGGCTGGAGC |
| | | CAGAGGACTTCGCCGTGTACTACTGCCAGCAGTACGCGGGGTCCCCGCC |
| | | GTTCACGTTCGGACAGGGAACCAAGGTCGAGATCAAG |
| 139114-aa | 83 | EVQLVESGGGLVQPGGSLRLSCAVSGFALSNHGMSWVRRAPGKGLEWVS |
| VH | | GIVYSGSTYYAASVKGRFTISRDNSRNTLYLQMNSLRPEDTAIYYCSAH |
| | | GGESDVWGQGTTVTVSS |
| 139114-aa | 98 | EIVLTQSPGTLSLSPGERATLSCRASQSIGSSSLAWYQQKPGQAPRLLM |
| VL | | YGASSRASGIPDRFSGSGSGTDFTLTISRLEPEDFAVYYCQQYAGSPPF |
| | | TFGQGTKVEIK |
| 139114-aa | 113 | MALPVTALLLPLALLLHAARPEVQLVESGGGLVQPGGSLRLSCAVSGFA |
| Full CAR | | LSNHGMSWVRRAPGKGLEWVSGIVYSGSTYYAASVKGRFTISRDNSRNT |
| | | LYLQMNSLRPEDTAIYYCSAHGGESDVWGQGTTVTVSSASGGGGSGGRA |
| | | SGGGGSEIVLTQSPGTLSLSPGERATLSCRASQSIGSSSLAWYQQKPGQ |
| | | APRLLMYGASSRASGIPDRFSGSGSGTDFTLTISRLEPEDFAVYYCQQY |
| | | AGSPPFTFGQGTKVEIKTTTPAPRPPTPAPTIASQPLSLRPEACRPAAG |
| | | GAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQ |
| | | LYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 139114-nt | 128 | ATGGCCCTCCCTGTCACCGCCCTGCTGCTTCCGCTGGCTCTTCTGCTCC |
| Full CAR | | ACGCCGCTCGGCCCGAAGTGCAATTGGTGGAATCTGGTGGAGGACTTGT |
| | | GCAACCTGGAGGATCACTGAGACTGTCATGCGCGGTGTCCGGTTTTGCC |
| | | CTGAGCAATCATGGGATGTCGTGGGTCCGGCGCGCCCCCGGAAAGGGTC |
| | | TGGAATGGGTGTCGGGTATCGTCTACTCCGGGAGCACTTACTACGCCGC |
| | | GAGCGTGAAGGGCCGCTTCACCATTTCCCGCGATAACTCCCGCAACACC |
| | | CTGTACTTGCAAATGAACTCGCTCCGGCCTGAGGACACTGCCATCTACT |
| | | ACTGCTCCGCACACGGAGGAGAATCCGACGTGTGGGGCCAGGGAACTAC |
| | | CGTGACCGTCAGCAGCGCCTCCGGCGGCGGGGGCTCAGGCGGACGGGCT |
| | | AGCGGCGGCGGTGGCTCCGAGATCGTGCTGACCCAGTCGCCTGGCACTC |
| | | TCTCGCTGAGCCCCGGGGAAAGGGCAACCCTGTCCTGTCGGGCCAGCCA |
| | | GTCCATTGGATCATCCTCCCTCGCCTGGTATCAGCAGAAACCGGGACAG |
| | | GCTCCGCGGCTGCTTATGTATGGGGCCAGCTCAAGAGCCTCCGGCATTC |
| | | CCGACCGGTTCTCCGGGTCCGGTTCCGGCACCGATTTCACCCTGACTAT |
| | | CTCGAGGCTGGAGCCAGAGGACTTCGCCGTGTACTACTGCCAGCAGTAC |
| | | GCGGGGTCCCCGCCGTTCACGTTCGGACAGGGAACCAAGGTCGAGATCA |
| | | AGACCACTACCCCAGCACCGAGGCCACCCACCCCGGCTCCTACCATCGC |
| | | CTCCCAGCCTCTGTCCCTGCGTCCGGAGGCATGTAGACCCGCAGCTGGT |
| | | GGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTT |
| | | CATGCCGGTTCCCAGAGGAGGAGGAAGGCGGCTGCGAACTGCGCGTGAA |
| | | ATTCAGCCGCAGCGCAGATGCTCCAGCCTACAAGCAGGGGCAGAACCAG |
| | | CTCTACAACGAACTCAATCTTGGTCGGAGAGAGGAGTACGACGTGCTGG |
| | | ACAAGCGGAGAGGACGGGACCCAGAAATGGGCGGGAAGCCGCGCAGAAA |
| | | GAATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAAG |
| | | GCCACGACGGACTGTACCAGGGACTCAGCACCGCCACCAAGGACACCTA |
| | | TGACGCTCTTCACATGCAGGCCCTGCCGCCTCGG |

149362

| | | |
|---|---|---|
| 149362-aa | 129 | QVQLQESGPGLVKPSETLSLTCTVSGGSISSSYYYWGWIRQPPGKGLEW |
| ScFv domain | | IGSIYYSGSAYYNPSLKSRVTISVDTSKNQFSLRLSSVTAADTAVYYCA |
| | | RHWQEWPDAFDIWGQGTMVTVSSGGGGSGGGGSGGGGSETTLTQSPAFM |
| | | SATPGDKVIISCKASQDIDDAMNWYQQKPGEAPLFIIQSATSPVPGIPP |
| | | RFSGSGFGTDFSLTINNIESEDAAYYFCLQHDNFPLTFGQGTKLEIK |
| 149362-nt | 150 | CAAGTGCAGCTTCAGGAAAGCGGACCGGGCCTGGTCAAGCCATCCGAAA |
| ScFv domain | | CTCTCTCCCTGACTTGCACTGTGTCTGGCGGTTCCATCTCATCGTCGTA |
| | | CTACTACTGGGGCTGGATTAGGCAGCCGCCCGGAAAGGGACTGGAGTGG |
| | | ATCGGAAGCATCTACTATTCCGGCTCGGCGTACTACAACCCTAGCCTCA |
| | | AGTCGAGAGTGACCATCTCCGTGGATACCTCCAAGAACCAGTTTTCCCT |
| | | GCGCCTGAGCTCCGTGACCGCCGCTGACACCGCCGTGTACTACTGTGCT |
| | | CGGCATTGGCAGGAATGGCCCGATGCCTTCGACATTTGGGGCCAGGGCA |
| | | CTATGGTCACTGTGTCATCCGGGGGTGGAGGCAGCGGGGGAGGAGGGTC |
| | | CGGGGGGGGAGGTTCAGAGACAACCTTGACCCAGTCACCCGCATTCATG |
| | | TCCGCCACTCCGGGAGACAAGGTCATCATCTCGTGCAAAGCGTCCCAGG |
| | | ATATCGACGATGCCATGAATTGGTACCAGCAGAAGCCTGGCGAAGCGCC |
| | | GCTGTTCATTATCCAATCCGCAACCTCGCCCGTGCCTGGAATCCCACCG |
| | | CGGTTCAGCGGCAGCGGTTTCGGAACCGACTTTTCCCTGACCATTAACA |
| | | ACATTGAGTCCGAGGACGCCGCCTACTACTTCTGCCTGCAACACGACAA |
| | | CTTCCCTCTCACGTTCGGCCAGGGAACCAAGCTGGAAATCAAG |
| 149362-aa | 171 | QVQLQESGPGLVKPSETLSLTCTVSGGSISSSYYYWGWIRQPPGKGLEW |
| VH | | IGSIYYSGSAYYNPSLKSRVTISVDTSKNQFSLRLSSVTAADTAVYYCA |
| | | RHWQEWPDAFDIWGQGTMVTVSS |
| 149362-aa VL | 192 | ETTLTQSPAFMSATPGDKVIISCKASQDIDDAMNWYQQKPGEAPLFIIQ |
| | | SATSPVPGIPPRFSGSGFGTDFSLTINNIESEDAAYYFCLQHDNFPLTF |
| | | GQGTKLEIK |
| 149362-aa | 213 | MALPVTALLLPLALLLHAARPQVQLQESGPGLVKPSETLSLTCTVSGGS |
| Full CAR | | ISSSYYYWGWIRQPPGKGLEWIGSIYYSGSAYYNPSLKSRVTISVDTSK |
| | | NQFSLRLSSVTAADTAVYYCARHWQEWPDAFDIWGQGTMVTVSSGGGGS |
| | | GGGGSGGGGSETTLTQSPAFMSATPGDKVIISCKASQDIDDAMNWYQQK |
| | | PGEAPLFIIQSATSPVPGIPPRFSGSGFGTDFSLTINNIESEDAAYYFC |
| | | LQHDNFPLTFGQGTKLEIKTTTPAPRPPTPAPTIASQPLSLRPEACRPA |
| | | AGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 149362-nt | 234 | ATGGCCCTCCCTGTCACCGCCCTGCTGCTTCCGCTGGCTCTTCTGCTCC |
| Full CAR | | ACGCCGCTCGGCCCCAAGTGCAGCTTCAGGAAAGCGGACCGGGCCTGGT |
| | | CAAGCCATCCGAAACTCTCTCCCTGACTTGCACTGTGTCTGGCGGTTCC |
| | | ATCTCATCGTCGTACTACTACTGGGGCTGGATTAGGCAGCCGCCCGGAA |
| | | AGGGACTGGAGTGGATCGGAAGCATCTACTATTCCGGCTCGGCGTACTA |
| | | CAACCCTAGCCTCAAGTCGAGAGTGACCATCTCCGTGGATACCTCCAAG |
| | | AACCAGTTTTCCCTGCGCCTGAGCTCCGTGACCGCCGCTGACACCGCCG |
| | | TGTACTACTGTGCTCGGCATTGGCAGGAATGGCCCGATGCCTTCGACAT |
| | | TTGGGGCCAGGGCACTATGGTCACTGTGTCATCCGGGGGTGGAGGCAGC |
| | | GGGGGAGGAGGGTCCGGGGGGGGAGGTTCAGAGACAACCTTGACCCAGT |
| | | CACCCGCATTCATGTCCGCCACTCCGGGAGACAAGGTCATCATCTCGTG |
| | | CAAAGCGTCCCAGGATATCGACGATGCCATGAATTGGTACCAGCAGAAG |
| | | CCTGGCGAAGCGCCGCTGTTCATTATCCAATCCGCAACCTCGCCCGTGC |
| | | CTGGAATCCCACCGCGGTTCAGCGGCAGCGGTTTCGGAACCGACTTTTC |
| | | CCTGACCATTAACAACATTGAGTCCGAGGACGCCGCCTACTACTTCTGC |
| | | CTGCAACACGACAACTTCCCTCTCACGTTCGGCCAGGGAACCAAGCTGG |
| | | AAATCAAGACCACTACCCCAGCACCGAGGCCACCCACCCCGGCTCCTAC |
| | | CATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCT |
| | | ACATTTGGGCCCCTCTGGCTGGTACTTGCGGGGTCCTGCTGCTTTCACT |
| | | CGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACG |
| | | GCTGTTCATGCCGGTTCCCAGAGGAGGAGGAAGGCGGCTGCGAACTGCG |
| | | CGTGAAATTCAGCCGCAGCGCAGATGCTCCAGCCTACAAGCAGGGGCAG |
| | | AACCAGCTCTACAACGAACTCAATCTTGGTCGGAGAGAGGAGTACGACG |
| | | TGCTGGACAAGCGGAGAGGACGGGACCCAGAAATGGGCGGGAAGCCGCG |
| | | CAGAAAGAATCCCCAAGAGGGCCTGTACAACGAGCTCCAAAAGGATAAG |
| | | ATGGCAGAAGCCTATAGCGAGATTGGTATGAAAGGGGAACGCAGAAGAG |
| | | GCAAAGGCCACGACGGACTGTACCAGGGACTCAGCACCGCCACCAAGGA |
| | | CACCTATGACGCTCTTCACATGCAGGCCCTGCCGCCTCGG |

149363

| | | |
|---|---|---|
| 149363-aa | 130 | VNLRESGPALVKPTQTLTLTCTFSGFSLRTSGMCVSWIRQPPGKALEWL |
| ScFv domain | | ARIDWDEDKFYSTSLKTRLTISKDTSDNQVVLRMTNMDPADTATYYCAR |
| | | SGAGGTSATAFDIWGPGTMVTVSSGGGGSGGGGSGGGGSDIQMTQSPSS |
| | | LSASVGDRVTITCRASQDIYNNLAWFQLKPGSAPRSLMYAANKSQSGVP |
| | | SRFSGSASGTDFTLTISSLQPEDFATYYCQHYYRFPYSFGQGTKLEIK |
| 149363-nt | 151 | CAAGTCAATCTGCGCGAATCCGGCCCCGCCTTGGTCAAGCCTACCCAGA |
| ScFv domain | | CCCTCACTCTGACCTGTACTTTCTCCGGCTTCTCCCTGCGGACTTCCGG |
| | | GATGTGCGTGTCCTGGATCAGACAGCCTCCGGGAAAGGCCCTGGAGTGG |
| | | CTCGCTCGCATTGACTGGGATGAGGACAAGTTCTACTCCACCTCACTCA |
| | | AGACCAGGCTGACCATCAGCAAAGATACCTCTGACAACCAAGTGGTGCT |
| | | CCGCATGACCAACATGGACCCAGCCGACACTGCCACTTACTACTGCGCG |
| | | AGGAGCGGAGCGGGCGGAACCTCCGCCACCGCCTTCGATATTTGGGGCC |
| | | CGGGTACCATGGTCACCGTGTCAAGCGGAGGAGGGGGGTCCGGGGGCGG |
| | | CGGTTCCGGGGGAGGCGGATCGGACATTCAGATGACTCAGTCACCATCG |
| | | TCCCTGAGCGCTAGCGTGGGCGACAGAGTGACAATCACTTGCCGGGCAT |
| | | CCCAGGACATCTATAACAACCTTGCGTGGTTCCAGCTGAAGCCTGGTTC |
| | | CGCACCGCGGTCACTTATGTACGCCGCCAACAAGAGCCAGTCGGGAGTG |
| | | CCGTCCCGGTTTTCCGGTTCGGCCTCGGGAACTGACTTCACCCTGACGA |
| | | TCTCCAGCCTGCAACCCGAGGATTTCGCCACCTACTACTGCCAGCACTA |
| | | CTACCGCTTTCCCTACTCGTTCGGACAGGGAACCAAGCTGGAAATCAAG |
| 149363-aa | 172 | QVNLRESGPALVKPTQTLTLTCTFSGFSLRTSGMCVSWIRQPPGKALEW |
| VH | | LARIDWDEDKFYSTSLKTRLTISKDTSDNQVVLRMTNMDPADTATYYCA |
| | | RSGAGGTSATAFDIWGPGTMVTVSS |
| 149363-aa VL | 193 | DIQMTQSPSSLSASVGDRVTITCRASQDIYNNLAWFQLKPGSAPRSLMY |
| | | AANKSQSGVPSRFSGSASGTDFTLTISSLQPEDFATYYCQHYYRFPYSF |
| | | GQGTKLEIK |
| 149363-aa | 214 | MALPVTALLLPLALLLHAARPQVNLRESGPALVKPTQTLTLTCTFSGFS |
| Full CAR | | LRTSGMCVSWIRQPPGKALEWLARIDWDEDKFYSTSLKTRLTISKDTSD |
| | | NQVVLRMTNMDPADTATYYCARSGAGGTSATAFDIWGPGTMVTVSSGGG |
| | | GSGGGGSGGGGSDIQMTQSPSSLSASVGDRVTITCRASQDIYNNLAWFQ |
| | | LKPGSAPRSLMYAANKSQSGVPSRFSGSASGTDFTLTISSLQPEDFATY |
| | | YCQHYYRFPYSFGQGTKLEIKTTTPAPRPPTPAPTIASQPLSLRPEACR |
| | | PAAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNELQK |
| | | DKMAEAYSEIGMKGERRRGKGHDGLYQGLSTATKDTYDALHMQALPPR |
| 149363-nt | 235 | ATGGCCCTCCCTGTCACCGCCCTGCTGCTTCCGCTGGCTCTTCTGCTCC |
| Full CAR | | ACGCCGCTCGGCCCCAAGTCAATCTGCGCGAATCCGGCCCCGCCTTGGT |
| | | CAAGCCTACCCAGACCCTCACTCTGACCTGTACTTTCTCCGGCTTCTCC |
| | | CTGCGGACTTCCGGGATGTGCGTGTCCTGGATCAGACAGCCTCCGGGAA |
| | | AGGCCCTGGAGTGGCTCGCTCGCATTGACTGGGATGAGGACAAGTTCTA |
| | | CTCCACCTCACTCAAGACCAGGCTGACCATCAGCAAAGATACCTCTGAC |
| | | AACCAAGTGGTGCTCCGCATGACCAACATGGACCCAGCCGACACTGCCA |
| | | CTTACTACTGCGCGAGGAGCGGAGCGGGCGGAACCTCCGCCACCGCCTT |
| | | CGATATTTGGGGCCCGGGTACCATGGTCACCGTGTCAAGCGGAGGAGGG |
| | | GGGTCCGGGGGCGGCGGTTCCGGGGGAGGCGGATCGGACATTCAGATGA |
| | | CTCAGTCACCATCGTCCCTGAGCGCTAGCGTGGGCGACAGAGTGACAAT |
| | | CACTTGCCGGGCATCCCAGGACATCTATAACAACCTTGCGTGGTTCCAG |
| | | CTGAAGCCTGGTTCCGCACCGCGGTCACTTATGTACGCCGCCAACAAGA |
| | | GCCAGTCGGGAGTGCCGTCCCGGTTTTCCGGTTCGGCCTCGGGAACTGA |
| | | CTTCACCCTGACGATCTCCAGCCTGCAACCCGAGGATTTCGCCACCTAC |
| | | TACTGCCAGCACTACTACCGCTTTCCCTACTCGTTCGGACAGGGAACCA |
| | | AGCTGGAAATCAAGACCACTACCCCAGCACCGAGGCCACCCACCCCGGC |
| | | TCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCG |
| | | ATATCTACATTTGGGCCCCTCTGGCTGGTACTTGCGGGGTCCTGCTGCT |
| | | TTCACTCGTGATCACTCTTTACTGTAAGCGCGGTCGGAAGAAGCTGCTG |
| | | TACATCTTTAAGCAACCCTTCATGAGGCCTGTGCAGACTACTCAAGAGG |
| | | AGGACGGCTGTTCATGCCGGTTCCCAGAGGAGGAGGAAGGCGGCTGCGA |
| | | ACTGCGCGTGAAATTCAGCCGCAGCGCAGATGCTCCAGCCTACAAGCAG |
| | | GGGCAGAACCAGCTCTACAACGAACTCAATCTTGGTCGGAGAGAGGAGT |
| | | ACGACGTGCTGGACAAGCGGAGAGGACGGGACCCAGAAATGGGCGGGAA |
| | | GCCGCGCAGAAAGAATCCCCAAGAGGGCCTGTACAACGAGCTCCAAAAG |
| | | GATAAGATGGCAGAAGCCTATAGCGAGATTGGTATGAAAGGGGAACGCA |
| | | GAAGAGGCAAAGGCCACGACGGACTGTACCAGGGACTCAGCACCGCCAC |
| | | CAAGGACACCTATGACGCTCTTCACATGCAGGCCCTGCCGCCTCGG |

149364

| | | |
|---|---|---|
| 149364-aa | 131 | EVQLVESGGGLVKPGGSLRLSCAASGFTFSSYSMNWVRQAPGKGLEWVS |
| ScFv domain | | sissSSSYIYYADSVKGRFTISRDNAKNSLYLQMNSLRAEDTAVYYCAK |
| | | TIAAVYAFDIWGQGTTVTVSSGGGGSGGGGSGGGGSEIVLTQSPLSLPV |
| | | TPEEPASISCRSSQSLLHSNGYNYLDWYLQKPGQSPQLLIYLGSNRASG |
| | | VPDRFSGSGSGTDFTLKISRVEAEDVGVYYCMQALQTPYTFGQGTKLEI |
| | | K |
| 149364-nt | 152 | GAAGTGCAGCTTGTCGAATCCGGGGGGGGACTGGTCAAGCCGGGCGGAT |
| ScFv domain | | CACTGAGACTGTCCTGCGCCGCGAGCGGCTTCACGTTCTCCTCCTACTC |
| | | CATGAACTGGGTCCGCCAAGCCCCCGGGAAGGGACTGGAATGGGTGTCC |
| | | TCTATCTCCTCGTCGTCGTCCTACATCTACTACGCCGACTCCGTGAAGG |
| | | GAAGATTCACCATTTCCCGCGACAACGCAAAGAACTCACTGTACTTGCA |
| | | AATGAACTCACTCCGGGCCGAAGATACTGCTGTGTACTATTGCGCCAAG |
| | | ACTATTGCCGCCGTCTACGCTTTCGACATCTGGGGCCAGGGAACCACCG |
| | | TGACTGTGTCGTCCGGTGGTGGTGGCTCGGGCGGAGGAGGAAGCGGCGG |
| | | CGGGGGGTCCGAGATTGTGCTGACCCAGTCGCCACTGAGCCTCCCTGTG |
| | | ACCCCCGAGGAACCCGCCAGCATCAGCTGCCGGTCCAGCCAGTCCCTGC |
| | | TCCACTCCAACGGATACAATTACCTCGATTGGTACCTTCAGAAGCCTGG |
| | | ACAAAGCCCGCAGCTGCTCATCTACTTGGGATCAAACCGCGCGTCAGGA |
| | | GTGCCTGACCGGTTCTCCGGCTCGGGCAGCGGTACCGATTTCACCCTGA |
| | | AAATCTCCAGGGTGGAGGCAGAGGACGTGGGAGTGTATTACTGTATGCA |
| | | GGCGCTGCAGACTCCGTACACATTTGGGCAGGGCACCAAGCTGGAGATC |
| | | AAG |
| 149364-aa | 173 | EVQLVESGGGLVKPGGSLRLSCAASGFTFSSYSMNWVRQAPGKGLEWVS |
| VH | | SISSSSSYIYYADSVKGRFTISRDNAKNSLYLQMNSLRAEDTAVYYCAK |
| | | TIAAVYAFDIWGQGTTVTVSS |
| 149364-aa VL | 194 | EIVLTQSPLSLPVTPEEPASISCRSSQSLLHSNGYNYLDWYLQKPGQSP |
| | | QLLIYLGSNRASGVPDRFSGSGSGTDFTLKISRVEAEDVGVYYCMQALQ |
| | | TPYTFGQGTKLEIK |
| 149364-aa | 215 | MALPVTALLLPLALLLHAARPEVQLVESGGGLVKPGGSLRLSCAASGFT |
| Full CAR | | FSSYSMNWVRQAPGKGLEWVSSISSSSSYIYYADSVKGRFTISRDNAKN |
| | | SLYLQMNSLRAEDTAVYYCAKTIAAVYAFDIWGQGTTVTVSSGGGGSGG |
| | | GGSGGGGSEIVLTQSPLSLPVTPEEPASISCRSSQSLLHSNGYNYLDWY |
| | | LQKPGQSPQLLIYLGSNRASGVPDRFSGSGSGTDFTLKISRVEAEDVGV |
| | | YYCMQALQTPYTFGQGTKLEIKTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 149364-nt | 236 | ATGGCCCTCCCTGTCACCGCCCTGCTGCTTCCGCTGGCTCTTCTGCTCC |
| Full CAR | | ACGCCGCTCGGCCCGAAGTGCAGCTTGTCGAATCCGGGGGGGGACTGGT |
| | | CAAGCCGGGCGGATCACTGAGACTGTCCTGCGCCGCGAGCGGCTTCACG |
| | | TTCTCCTCCTACTCCATGAACTGGGTCCGCCAAGCCCCCGGGAAGGGAC |
| | | TGGAATGGGTGTCCTCTATCTCCTCGTCGTCGTCCTACATCTACTACGC |
| | | CGACTCCGTGAAGGGAAGATTCACCATTTCCCGCGACAACGCAAAGAAC |
| | | TCACTGTACTTGCAAATGAACTCACTCCGGGCCGAAGATACTGCTGTGT |
| | | ACTATTGCGCCAAGACTATTGCCGCCGTCTACGCTTTCGACATCTGGGG |
| | | CCAGGGAACCACCGTGACTGTGTCGTCCGGTGGTGGTGGCTCGGGCGGA |
| | | GGAGGAAGCGGCGGCGGGGGGTCCGAGATTGTGCTGACCCAGTCGCCAC |
| | | TGAGCCTCCCTGTGACCCCCGAGGAACCCGCCAGCATCAGCTGCCGGTC |
| | | CAGCCAGTCCCTGCTCCACTCCAACGGATACAATTACCTCGATTGGTAC |
| | | CTTCAGAAGCCTGGACAAAGCCCGCAGCTGCTCATCTACTTGGGATCAA |
| | | ACCGCGCGTCAGGAGTGCCTGACCGGTTCTCCGGCTCGGGCAGCGGTAC |
| | | CGATTTCACCCTGAAAATCTCCAGGGTGGAGGCAGAGGACGTGGGAGTG |
| | | TATTACTGTATGCAGGCGCTGCAGACTCCGTACACATTTGGGCAGGGCA |
| | | CCAAGCTGGAGATCAAGACCACTACCCCAGCACCGAGGCCACCCACCCC |
| | | GGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAG |
| | | AGGAGGACGGCTGTTCATGCCGGTTCCCAGAGGAGGAGGAAGGCGGCTG |
| | | CGAACTGCGCGTGAAATTCAGCCGCAGCGCAGATGCTCCAGCCTACAAG |
| | | CAGGGGCAGAACCAGCTCTACAACGAACTCAATCTTGGTCGGAGAGAGG |
| | | AGTACGACGTGCTGGACAAGCGGAGAGGACGGGACCCAGAAATGGGCGG |
| | | GAAGCCGCGCAGAAAGAATCCCCAAGAGGGCCTGTACAACGAGCTCCAA |
| | | AAGGATAAGATGGCAGAAGCCTATAGCGAGATTGGTATGAAAGGGGAAC |
| | | GCAGAAGAGGCAAAGGCCACGACGGACTGTACCAGGGACTCAGCACCGC |
| | | CACCAAGGACACCTATGACGCTCTTCACATGCAGGCCCTGCCGCCTCGG |

149365

| | | |
|---|---|---|
| 149365-aa | 132 | EVQLVESGGGLVKPGGSLRLSCAASGFTFSDYYMSWIRQAPGKGLEWVS |
| ScFv domain | | YISSSGSTIYYADSVKGRFTISRDNAKNSLYLQMNSLRAEDTAVYYCAR |
| | | DLRGAFDIWGQGTMVTVSSGGGGSGGGGSGGGGSSYVLTQSPSVSAAPG |
| | | YTATISCGGNNIGTKSVHWYQQKPGQAPLLVIRDDSVRPSKIPGRFSGS |
| | | NSGNMATLTISGVQAGDEADFYCQVWDSDSEHVVFGGGTKLTVL |
| 149365-nt | 153 | GAAGTCCAGCTCGTGGAGTCCGGCGGAGGCCTTGTGAAGCCTGGAGGTT |
| ScFv domain | | CGCTGAGACTGTCCTGCGCCGCCTCCGGCTTCACCTTCTCCGACTACTA |
| | | CATGTCCTGGATCAGACAGGCCCCGGGAAAGGGCCTGGAATGGGTGTCC |
| | | TACATCTCGTCATCGGGCAGCACTATCTACTACGCGGACTCAGTGAAGG |
| | | GGCGGTTCACCATTTCCCGGGATAACGCGAAGAACTCGCTGTATCTGCA |
| | | AATGAACTCACTGAGGGCCGAGGACACCGCCGTGTACTACTGCGCCCGC |
| | | GATCTCCGCGGGGCATTTGACATCTGGGGACAGGGAACCATGGTCACAG |
| | | TGTCCAGCGGAGGGGGAGGATCGGGTGGCGGAGGTTCCGGGGGTGGAGG |
| | | CTCCTCCTACGTGCTGACTCAGAGCCCAAGCGTCAGCGCTGCGCCCGGT |
| | | TACACGGCAACCATCTCCTGTGGCGGAAACAACATTGGGACCAAGTCTG |
| | | TGCACTGGTATCAGCAGAAGCCGGGCCAAGCTCCCCTGTTGGTGATCCG |
| | | CGATGACTCCGTGCGGCCTAGCAAAATTCCGGGACGGTTCTCCGGCTCC |
| | | AACAGCGGCAATATGGCCACTCTCACCATCTCGGGAGTGCAGGCCGGAG |
| | | ATGAAGCCGACTTCTACTGCCAAGTCTGGGACTCAGACTCCGAGCATGT |
| | | GGTGTTCGGGGGCGGAACCAAGCTGACTGTGCTC |
| 149365-aa | 174 | EVQLVESGGGLVKPGGSLRLSCAASGFTFSDYYMSWIRQAPGKGLEWVS |
| VH | | YISSSGSTIYYADSVKGRFTISRDNAKNSLYLQMNSLRAEDTAVYYCAR |
| | | DLRGAFDIWGQGTMVTVSS |
| 149365-aa VL | 195 | SYVLTQSPSVSAAPGYTATISCGGNNIGTKSVHWYQQKPGQAPLLVIRD |
| | | DSVRPSKIPGRFSGSNSGNMATLTISGVQAGDEADFYCQVWDSDSEHVV |
| | | FGGGTKLTVL |
| 149365-aa | 216 | MALPVTALLLPLALLLHAARPEVQLVESGGGLVKPGGSLRLSCAASGFT |
| Full CAR | | FSDYYMSWIRQAPGKGLEWVSYISSSGSTIYYADSVKGRFTISRDNAKN |
| | | SLYLQMNSLRAEDTAVYYCARDLRGAFDIWGQGTMVTVSSGGGGSGGGG |
| | | SGGGGSSYVLTQSPSVSAAPGYTATISCGGNNIGTKSVHWYQQKPGQAP |
| | | LLVIRDDSVRPSKIPGRFSGSNSGNMATLTISGVQAGDEADFYCQVWDS |
| | | DSEHVVFGGGTKLTVLTTTPAPRPPTPAPTIASQPLSLRPEACRPAAGG |
| | | AVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQL |
| | | YNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 149365-nt | 237 | ATGGCCCTCCCTGTCACCGCCCTGCTGCTTCCGCTGGCTCTTCTGCTCC |
| Full CAR | | ACGCCGCTCGGCCCGAAGTCCAGCTCGTGGAGTCCGGCGGAGGCCTTGT |
| | | GAAGCCTGGAGGTTCGCTGAGACTGTCCTGCGCCGCCTCCGGCTTCACC |
| | | TTCTCCGACTACTACATGTCCTGGATCAGACAGGCCCCGGGAAAGGGCC |
| | | TGGAATGGGTGTCCTACATCTCGTCATCGGGCAGCACTATCTACTACGC |
| | | GGACTCAGTGAAGGGGCGGTTCACCATTTCCCGGGATAACGCGAAGAAC |
| | | TCGCTGTATCTGCAAATGAACTCACTGAGGGCCGAGGACACCGCCGTGT |
| | | ACTACTGCGCCCGCGATCTCCGCGGGGCATTTGACATCTGGGGACAGGG |
| | | AACCATGGTCACAGTGTCCAGCGGAGGGGGAGGATCGGGTGGCGGAGGT |
| | | TCCGGGGGTGGAGGCTCCTCCTACGTGCTGACTCAGAGCCCAAGCGTCA |
| | | GCGCTGCGCCCGGTTACACGGCAACCATCTCCTGTGGCGGAAACAACAT |
| | | TGGGACCAAGTCTGTGCACTGGTATCAGCAGAAGCCGGGCCAAGCTCCC |
| | | CTGTTGGTGATCCGCGATGACTCCGTGCGGCCTAGCAAAATTCCGGGAC |
| | | GGTTCTCCGGCTCCAACAGCGGCAATATGGCCACTCTCACCATCTCGGG |
| | | AGTGCAGGCCGGAGATGAAGCCGACTTCTACTGCCAAGTCTGGGACTCA |
| | | GACTCCGAGCATGTGGTGTTCGGGGGCGGAACCAAGCTGACTGTGCTCA |
| | | CCACTACCCCAGCACCGAGGCCACCCACCCCGGCTCCTACCATCGCCTC |
| | | CCAGCCTCTGTCCCTGCGTCCGGAGGCATGTAGACCCGCAGCTGGTGGG |
| | | GCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCAC |
| | | TCTTTACTGTAAGCGCGGTCGGAAGAAGCTGCTGTACATCTTTAAGCAA |
| | | CCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATT |
| | | CAGCCGCAGCGCAGATGCTCCAGCCTACAAGCAGGGGCAGAACCAGCTC |
| | | TACAACGAACTCAATCTTGGTCGGAGAGAGGAGTACGACGTGCTGGACA |
| | | AGCGGAGAGGACGGGACCCAGAAATGGGCGGGAAGCCGCGCAGAAAGAA |
| | | TCCCCAAGAGGGCCTGTACAACGAGCTCCAAAAGGATAAGATGGCAGAA |
| | | GCCTATAGCGAGATTGGTATGAAAGGGGAACGCAGAAGAGGCAAAGGCC |
| | | ACGACGGACTGTACCAGGGACTCAGCACCGCCACCAAGGACACCTATGA |
| | | CGCTCTTCACATGCAGGCCCTGCCGCCTCGG |

149366

| | | |
|---|---|---|
| 149366-aa | 133 | QVQLVQSGAEVKKPGASVKVSCKPSGYTVTSHYIHWVRRAPGQGLEWMG |
| ScFv domain | | MINPSGGVTAYSQTLQGRVTMTSDTSSSTVYMELSSLRSEDTAMYYCAR |
| | | EGSGSGWYFDFWGRGTLVTVSSGGGGSGGGGSGGGGSSYVLTQPPSVSV |
| | | SPGQTASITCSGDGLSKKYVSWYQQKAGQSPVVLISRDKERPSGIPDRF |
| | | SGSNSADTATLTISGTQAMDEADYYCQAWDDTTVVFGGGTKLTVL |
| 149366-nt | 154 | CAAGTGCAGCTGGTGCAGAGCGGGGCCGAAGTCAAGAAGCCGGGAGCCT |
| ScFv domain | | CCGTGAAAGTGTCCTGCAAGCCTTCGGGATACACCGTGACCTCCCACTA |
| | | CATTCATTGGGTCCGCCGCGCCCCCGGCCAAGGACTCGAGTGGATGGGC |
| | | ATGATCAACCCTAGCGGCGGAGTGACCGCGTACAGCCAGACGCTGCAGG |
| | | GACGCGTGACTATGACCTCGGATACCTCCTCCTCCACCGTCTATATGGA |
| | | ACTGTCCAGCCTGCGGTCCGAGGATACCGCCATGTACTACTGCGCCCGG |
| | | GAAGGATCAGGCTCCGGGTGGTATTTCGACTTCTGGGGAAGAGGCACCC |
| | | TCGTGACTGTGTCATCTGGGGGAGGGGGTTCCGGTGGTGGCGGATCGGG |
| | | AGGAGGCGGTTCATCCTACGTGCTGACCCAGCCACCCTCCGTGTCCGTG |
| | | AGCCCCGGCCAGACTGCATCGATTACATGTAGCGGCGACGGCCTCTCCA |
| | | AGAAATACGTGTCGTGGTACCAGCAGAAGGCCGGACAGAGCCCGGTGGT |
| | | GCTGATCTCAAGAGATAAGGAGCGGCCTAGCGGAATCCCGGACAGGTTC |
| | | TCGGGTTCCAACTCCGCGGACACTGCTACTCTGACCATCTCGGGGACCC |
| | | AGGCTATGGACGAAGCCGATTACTACTGCCAAGCCTGGGACGACACTAC |
| | | TGTCGTGTTTGGAGGGGGCACCAAGTTGACCGTCCTT |
| 149366-aa | 175 | QVQLVQSGAEVKKPGASVKVSCKPSGYTVTSHYIHWVRRAPGQGLEWMG |
| VH | | MINPSGGVTAYSQTLQGRVTMTSDTSSSTVYMELSSLRSEDTAMYYCAR |
| | | EGSGSGWYFDFWGRGTLVTVSS |
| 149366-aa VL | 196 | SYVLTQPPSVSVSPGQTASITCSGDGLSKKYVSWYQQKAGQSPVVLISR |
| | | DKERPSGIPDRFSGSNSADTATLTISGTQAMDEADYYCQAWDDTTVVFG |
| | | GGTKLTVL |
| 149366-aa | 217 | MALPVTALLLPLALLLHAARPQVQLVQSGAEVKKPGASVKVSCKPSGYT |
| Full CAR | | VTSHYIHWVRRAPGQGLEWMGMINPSGGVTAYSQTLQGRVTMTSDTSSS |
| | | TVYMELSSLRSEDTAMYYCAREGSGSGWYFDFWGRGTLVTVSSGGGGSG |
| | | GGGSGGGGSSYVLTQPPSVSVSPGQTASITCSGDGLSKKYVSWYQQKAG |
| | | QSPVVLISRDKERPSGIPDRFSGSNSADTATLTISGTQAMDEADYYCQA |
| | | WDDTTVVFGGGTKLTVLTTTPAPRPPTPAPTIASQPLSLRPEACRPAAG |
| | | GAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQ |
| | | LYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 149366-nt | 238 | ATGGCCCTCCCTGTCACCGCCCTGCTGCTTCCGCTGGCTCTTCTGCTCC |
| Full CAR | | ACGCCGCTCGGCCCCAAGTGCAGCTGGTGCAGAGCGGGGCCGAAGTCAA |
| | | GAAGCCGGGAGCCTCCGTGAAAGTGTCCTGCAAGCCTTCGGGATACACC |
| | | GTGACCTCCCACTACATTCATTGGGTCCGCCGCGCCCCCGGCCAAGGAC |
| | | TCGAGTGGATGGGCATGATCAACCCTAGCGGCGGAGTGACCGCGTACAG |
| | | CCAGACGCTGCAGGGACGCGTGACTATGACCTCGGATACCTCCTCCTCC |
| | | ACCGTCTATATGGAACTGTCCAGCCTGCGGTCCGAGGATACCGCCATGT |
| | | ACTACTGCGCCCGGGAAGGATCAGGCTCCGGGTGGTATTTCGACTTCTG |
| | | GGGAAGAGGCACCCTCGTGACTGTGTCATCTGGGGGAGGGGGTTCCGGT |
| | | GGTGGCGGATCGGGAGGAGGCGGTTCATCCTACGTGCTGACCCAGCCAC |
| | | CCTCCGTGTCCGTGAGCCCCGGCCAGACTGCATCGATTACATGTAGCGG |
| | | CGACGGCCTCTCCAAGAAATACGTGTCGTGGTACCAGCAGAAGGCCGGA |
| | | CAGAGCCCGGTGGTGCTGATCTCAAGAGATAAGGAGCGGCCTAGCGGAA |
| | | TCCCGGACAGGTTCTCGGGTTCCAACTCCGCGGACACTGCTACTCTGAC |
| | | CATCTCGGGGACCCAGGCTATGGACGAAGCCGATTACTACTGCCAAGCC |
| | | TGGGACGACACTACTGTCGTGTTTGGAGGGGGCACCAAGTTGACCGTCC |
| | | TTACCACTACCCCAGCACCGAGGCCACCCACCCCGGCTCCTACCATCGC |
| | | CTCCCAGCCTCTGTCCCTGCGTCCGGAGGCATGTAGACCCGCAGCTGGT |
| | | GGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTT |
| | | CATGCCGGTTCCCAGAGGAGGAGGAAGGCGGCTGCGAACTGCGCGTGAA |
| | | ATTCAGCCGCAGCGCAGATGCTCCAGCCTACAAGCAGGGGCAGAACCAG |
| | | CTCTACAACGAACTCAATCTTGGTCGGAGAGAGGAGTACGACGTGCTGG |
| | | ACAAGCGGAGAGGACGGGACCCAGAAATGGGCGGGAAGCCGCGCAGAAA |
| | | GAATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAAG |
| | | GCCACGACGGACTGTACCAGGGACTCAGCACCGCCACCAAGGACACCTA |
| | | TGACGCTCTTCACATGCAGGCCCTGCCGCCTCGG |

149367

| | | |
|---|---|---|
| 149367-aa | 134 | QVQLQESGPGLVKPSQTLSLTCTVSGGSISSGGYYWSWIRQHPGKGLEW |
| ScFv domain | | IGYIYYSGSTYYNPSLKSRVTISVDTSKNQFSLKLSSVTAADTAVYYCA |
| | | RAGIAARLRGAFDIWGQGTMVTVSSGGGGSGGGGSGGGGSDIVMTQSPS |
| | | SVSASVGDRVIITCRASQGIRNWLAWYQQKPGKAPNLLIYAASNLQSGV |
| | | PSRFSGSGSGADFTLTISSLQPEDVATYYCQKYNSAPFTFGPGTKVDIK |
| 149367-nt | 155 | CAAGTGCAGCTTCAGGAGAGCGGCCCGGGACTCGTGAAGCCGTCCCAGA |
| ScFv domain | | CCCTGTCCCTGACTTGCACCGTGTCGGGAGGAAGCATCTCGAGCGGAGG |
| | | CTACTATTGGTCGTGGATTCGGCAGCACCCTGGAAAGGGCCTGGAATGG |
| | | ATCGGCTACATCTACTACTCCGGCTCGACCTACTACAACCCATCGCTGA |
| | | AGTCCAGAGTGACAATCTCAGTGGACACGTCCAAGAATCAGTTCAGCCT |
| | | GAAGCTCTCTTCCGTGACTGCGGCCGACACCGCCGTGTACTACTGCGCA |
| | | CGCGCTGGAATTGCCGCCCGGCTGAGGGGTGCCTTCGACATTTGGGGAC |
| | | AGGGCACCATGGTCACCGTGTCCTCCGGCGGCGGAGGTTCCGGGGGTGG |
| | | AGGCTCAGGAGGAGGGGGGTCCGACATCGTCATGACTCAGTCGCCCTCA |
| | | AGCGTCAGCGCGTCCGTCGGGGACAGAGTGATCATCACCTGTCGGGCGT |
| | | CCCAGGGAATTCGCAACTGGCTGGCCTGGTATCAGCAGAAGCCCGGAAA |
| | | GGCCCCCAACCTGTTGATCTACGCCGCCTCAAACCTCCAATCCGGGGTG |
| | | CCGAGCCGCTTCAGCGGCTCCGGTTCGGGTGCCGATTTCACTCTGACCA |
| | | TCTCCTCCCTGCAACCTGAAGATGTGGCTACCTACTACTGCCAAAAGTA |
| | | CAACTCCGCACCTTTTACTTTCGGACCGGGGACCAAAGTGGACATTAAG |
| 149367-aa | 176 | QVQLQESGPGLVKPSQTLSLTCTVSGGSISSGGYYWSWIRQHPGKGLEW |
| VH | | IGYIYYSGSTYYNPSLKSRVTISVDTSKNQFSLKLSSVTAADTAVYYCA |
| | | RAGIAARLRGAFDIWGQGTMVTVSS |
| 149367-aa VL | 197 | DIVMTQSPSSVSASVGDRVIITCRASQGIRNWLAWYQQKPGKAPNLLIY |
| | | AASNLQSGVPSRFSGSGSGADFTLTISSLQPEDVATYYCQKYNSAPFTF |
| | | GPGTKVDIK |
| 149367-aa | 218 | MALPVTALLLPLALLLHAARPQVQLQESGPGLVKPSQTLSLTCTVSGGS |
| Full CAR | | ISSGGYYWSWIRQHPGKGLEWIGYIYYSGSTYYNPSLKSRVTISVDTSK |
| | | NQFSLKLSSVTAADTAVYYCARAGIAARLRGAFDIWGQGTMVTVSSGGG |
| | | GSGGGGSGGGGSDIVMTQSPSSVSASVGDRVIITCRASQGIRNWLAWYQ |
| | | QKPGKAPNLLIYAASNLQSGVPSRFSGSGSGADFTLTISSLQPEDVATY |
| | | YCQKYNSAPFTFGPGTKVDIKTTTPAPRPPTPAPTIASQPLSLRPEACR |
| | | PAAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNELQK |
| | | DKMAEAYSEIGMKGERRRGKGHDGLYQGLSTATKDTYDALHMQALPPR |
| 149367-nt | 239 | ATGGCCCTCCCTGTCACCGCCCTGCTGCTTCCGCTGGCTCTTCTGCTCC |
| Full CAR | | ACGCCGCTCGGCCCCAAGTGCAGCTTCAGGAGAGCGGCCCGGGACTCGT |
| | | GAAGCCGTCCCAGACCCTGTCCCTGACTTGCACCGTGTCGGGAGGAAGC |
| | | ATCTCGAGCGGAGGCTACTATTGGTCGTGGATTCGGCAGCACCCTGGAA |
| | | AGGGCCTGGAATGGATCGGCTACATCTACTACTCCGGCTCGACCTACTA |
| | | CAACCCATCGCTGAAGTCCAGAGTGACAATCTCAGTGGACACGTCCAAG |
| | | AATCAGTTCAGCCTGAAGCTCTCTTCCGTGACTGCGGCCGACACCGCCG |
| | | TGTACTACTGCGCACGCGCTGGAATTGCCGCCCGGCTGAGGGGTGCCTT |
| | | CGACATTTGGGGACAGGGCACCATGGTCACCGTGTCCTCCGGCGGCGGA |
| | | GGTTCCGGGGGTGGAGGCTCAGGAGGAGGGGGGTCCGACATCGTCATGA |
| | | CTCAGTCGCCCTCAAGCGTCAGCGCGTCCGTCGGGGACAGAGTGATCAT |
| | | CACCTGTCGGGCGTCCCAGGGAATTCGCAACTGGCTGGCCTGGTATCAG |
| | | CAGAAGCCCGGAAAGGCCCCCAACCTGTTGATCTACGCCGCCTCAAACC |
| | | TCCAATCCGGGGTGCCGAGCCGCTTCAGCGGCTCCGGTTCGGGTGCCGA |
| | | TTTCACTCTGACCATCTCCTCCCTGCAACCTGAAGATGTGGCTACCTAC |
| | | TACTGCCAAAAGTACAACTCCGCACCTTTTACTTTCGGACCGGGGACCA |
| | | AAGTGGACATTAAGACCACTACCCCAGCACCGAGGCCACCCACCCCGGC |
| | | TCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCG |
| | | ATATCTACATTTGGGCCCCTCTGGCTGGTACTTGCGGGGTCCTGCTGCT |
| | | TTCACTCGTGATCACTCTTTACTGTAAGCGCGGTCGGAAGAAGCTGCTG |
| | | TACATCTTTAAGCAACCCTTCATGAGGCCTGTGCAGACTACTCAAGAGG |
| | | AGGACGGCTGTTCATGCCGGTTCCCAGAGGAGGAGGAAGGCGGCTGCGA |
| | | ACTGCGCGTGAAATTCAGCCGCAGCGCAGATGCTCCAGCCTACAAGCAG |
| | | GGGCAGAACCAGCTCTACAACGAACTCAATCTTGGTCGGAGAGAGGAGT |
| | | ACGACGTGCTGGACAAGCGGAGAGGACGGGACCCAGAAATGGGCGGGAA |
| | | GCCGCGCAGAAAGAATCCCCAAGAGGGCCTGTACAACGAGCTCCAAAAG |
| | | GATAAGATGGCAGAAGCCTATAGCGAGATTGGTATGAAAGGGGAACGCA |
| | | GAAGAGGCAAAGGCCACGACGGACTGTACCAGGGACTCAGCACCGCCAC |
| | | CAAGGACACCTATGACGCTCTTCACATGCAGGCCCTGCCGCCTCGG |

149368

| | | |
|---|---|---|
| 149368-aa | 135 | QVQLVQSGAEVKKPGSSVKVSCKASGGTFSSYAISWVRQAPGQGLEWMG |
| ScFv domain | | GIIPIFGTANYAQKFQGRVTITADESTSTAYMELSSLRSEDTAVYYCAR |
| | | RGGYQLLRWDVGLLRSAFDIWGQGTMVTVSSGGGGSGGGGSGGGGSSYV |
| | | LTQPPSVSVAPGQTARITCGGNNIGSKSVHWYQQKPGQAPVLVLYGKNN |
| | | RPSGVPDRFSGSRSGTTASLTITGAQAEDEADYYCSSRDSSGDHLRVFG |
| | | TGTKVTVL |
| 149368-nt | 156 | CAAGTGCAGCTGGTCCAGTCGGGCGCCGAGGTCAAGAAGCCCGGGAGCT |
| ScFv domain | | CTGTGAAAGTGTCCTGCAAGGCCTCCGGGGGCACCTTTAGCTCCTACGC |
| | | CATCTCCTGGGTCCGCCAAGCACCGGGTCAAGGCCTGGAGTGGATGGGG |
| | | GGAATTATCCCTATCTTCGGCACTGCCAACTACGCCCAGAAGTTCCAGG |
| | | GACGCGTGACCATTACCGCGGACGAATCCACCTCCACCGCTTATATGGA |
| | | GCTGTCCAGCTTGCGCTCGGAAGATACCGCCGTGTACTACTGCGCCCGG |
| | | AGGGGTGGATACCAGCTGCTGAGATGGGACGTGGGCCTCCTGCGGTCGG |
| | | CGTTCGACATCTGGGGCCAGGGCACTATGGTCACTGTGTCCAGCGGAGG |
| | | AGGCGGATCGGGAGGCGGCGGATCAGGGGGAGGCGGTTCCAGCTACGTG |
| | | CTTACTCAACCCCCTTCGGTGTCCGTGGCCCCGGGACAGACCGCCAGAA |
| | | TCACTTGCGGAGGAAACAACATTGGGTCCAAGAGCGTGCATTGGTACCA |
| | | GCAGAAGCCAGGACAGGCCCCTGTGCTGGTGCTCTACGGGAAGAACAAT |
| | | CGGCCCAGCGGAGTGCCGGACAGGTTCTCGGGTTCACGCTCCGGTACAA |
| | | CCGCTTCACTGACTATCACCGGGGCCCAGGCAGAGGATGAAGCGGACTA |
| | | CTACTGTTCCTCCCGGGATTCATCCGGCGACCACCTCCGGGTGTTCGGA |
| | | ACCGGAACGAAGGTCACCGTGCTG |
| 149368-aa | 177 | QVQLVQSGAEVKKPGSSVKVSCKASGGTFSSYAISWVRQAPGQGLEWMG |
| VH | | GIIPIFGTANYAQKFQGRVTITADESTSTAYMELSSLRSEDTAVYYCAR |
| | | RGGYQLLRWDVGLLRSAFDIWGQGTMVTVSS |
| 149368-aa VL | 198 | SYVLTQPPSVSVAPGQTARITCGGNNIGSKSVHWYQQKPGQAPVLVLYG |
| | | KNNRPSGVPDRFSGSRSGTTASLTITGAQAEDEADYYCSSRDSSGDHLR |
| | | VFGTGTKVTVL |
| 149368-aa | 219 | MALPVTALLLPLALLLHAARPQVQLVQSGAEVKKPGSSVKVSCKASGGT |
| Full CAR | | FSSYAISWVRQAPGQGLEWMGGIIPIFGTANYAQKFQGRVTITADESTS |
| | | TAYMELSSLRSEDTAVYYCARRGGYQLLRWDVGLLRSAFDIWGQGTMVT |
| | | VSSGGGGSGGGGSGGGGSSYVLTQPPSVSVAPGQTARITCGGNNIGSKS |
| | | VHWYQQKPGQAPVLVLYGKNNRPSGVPDRFSGSRSGTTASLTITGAQAE |
| | | DEADYYCSSRDSSGDHLRVFGTGTKVTVLTTTPAPRPPTPAPTIASQPL |
| | | SLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDALH |
| | | MQALPPR |
| 149368-nt | 240 | ATGGCCCTCCCTGTCACCGCCCTGCTGCTTCCGCTGGCTCTTCTGCTCC |
| Full CAR | | ACGCCGCTCGGCCCCAAGTGCAGCTGGTCCAGTCGGGCGCCGAGGTCAA |
| | | GAAGCCCGGGAGCTCTGTGAAAGTGTCCTGCAAGGCCTCCGGGGGCACC |
| | | TTTAGCTCCTACGCCATCTCCTGGGTCCGCCAAGCACCGGGTCAAGGCC |
| | | TGGAGTGGATGGGGGGAATTATCCCTATCTTCGGCACTGCCAACTACGC |
| | | CCAGAAGTTCCAGGGACGCGTGACCATTACCGCGGACGAATCCACCTCC |
| | | ACCGCTTATATGGAGCTGTCCAGCTTGCGCTCGGAAGATACCGCCGTGT |
| | | ACTACTGCGCCCGGAGGGGTGGATACCAGCTGCTGAGATGGGACGTGGG |
| | | CCTCCTGCGGTCGGCGTTCGACATCTGGGGCCAGGGCACTATGGTCACT |
| | | GTGTCCAGCGGAGGAGGCGGATCGGGAGGCGGCGGATCAGGGGGAGGCG |
| | | GTTCCAGCTACGTGCTTACTCAACCCCCTTCGGTGTCCGTGGCCCCGGG |
| | | ACAGACCGCCAGAATCACTTGCGGAGGAAACAACATTGGGTCCAAGAGC |
| | | GTGCATTGGTACCAGCAGAAGCCAGGACAGGCCCCTGTGCTGGTGCTCT |
| | | ACGGGAAGAACAATCGGCCCAGCGGAGTGCCGGACAGGTTCTCGGGTTC |
| | | ACGCTCCGGTACAACCGCTTCACTGACTATCACCGGGGCCCAGGCAGAG |
| | | GATGAAGCGGACTACTACTGTTCCTCCCGGGATTCATCCGGCGACCACC |
| | | TCCGGGTGTTCGGAACCGGAACGAAGGTCACCGTGCTGACCACTACCCC |
| | | AGCACCGAGGCCACCCACCCCGGCTCCTACCATCGCCTCCCAGCCTCTG |
| | | TCCCTGCGTCCGGAGGCATGTAGACCCGCAGCTGGTGGGGCCGTGCATA |
| | | CCCGGGGTCTTGACTTCGCCTGCGATATCTACATTTGGGCCCCTCTGGC |
| | | TGGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATGA |
| | | GGCCTGTGCAGACTACTCAAGAGGAGGACGGCTGTTCATGCCGGTTCCC |
| | | AGAGGAGGAGGAAGGCGGCTGCGAACTGCGCGTGAAATTCAGCCGCAGC |
| | | GCAGATGCTCCAGCCTACAAGCAGGGGCAGAACCAGCTCTACAACGAAC |
| | | TCAATCTTGGTCGGAGAGAGGAGTACGACGTGCTGGACAAGCGGAGAGG |
| | | ACGGGACCCAGAAATGGGCGGGAAGCCGCGCAGAAAGAATCCCCAAGAG |
| | | GGCCTGTACAACGAGCTCCAAAAGGATAAGATGGCAGAAGCCTATAGCG |
| | | AGATTGGTATGAAAGGGGAACGCAGAAGAGGCAAAGGCCACGACGGACT |
| | | GTACCAGGGACTCAGCACCGCCACCAAGGACACCTATGACGCTCTTCAC |
| | | ATGCAGGCCCTGCCGCCTCGG |

149369

| | | |
|---|---|---|
| 149369-aa | 136 | EVQLQQSGPGLVKPSQTLSLTCAISGDSVSSNSAAWNWIRQSPSRGLEW |
| ScFv domain | | LGRTYYRSKWYSFYAISLKSRIIINPDTSKNQFSLQLKSVTPEDTAVYY |
| | | CARSSPEGLFLYWFDPWGQGTLVTVSSGGDGSGGGGSGGGGSSSELTQD |
| | | PAVSVALGQTIRITCQGDSLGNYYATWYQQKPGQAPVLVIYGTNNRPSG |
| | | IPDRFSASSSGNTASLTITGAQAEDEADYYCNSRDSSGHHLLFGTGTKV |
| | | TVL |
| 149369-nt | 157 | GAAGTGCAGCTCCAACAGTCAGGACCGGGGCTCGTGAAGCCATCCCAGA |
| ScFv domain | | CCCTGTCCCTGACTTGTGCCATCTCGGGAGATAGCGTGTCATCGAACTC |
| | | CGCCGCCTGGAACTGGATTCGGCAGAGCCCGTCCCGCGGACTGGAGTGG |
| | | CTTGGAAGGACCTACTACCGGTCCAAGTGGTACTCTTTCTACGCGATCT |
| | | CGCTGAAGTCCCGCATTATCATTAACCCTGATACCTCCAAGAATCAGTT |
| | | CTCCCTCCAACTGAAATCCGTCACCCCCGAGGACACAGCAGTGTATTAC |
| | | TGCGCACGGAGCAGCCCCGAAGGACTGTTCCTGTATTGGTTTGACCCCT |
| | | GGGGCCAGGGGACTCTTGTGACCGTGTCGAGCGGCGGAGATGGGTCCGG |
| | | TGGCGGTGGTTCGGGGGGCGGCGGATCATCATCCGAACTGACCCAGGAC |
| | | CCGGCTGTGTCCGTGGCGCTGGGACAAACCATCCGCATTACGTGCCAGG |
| | | GAGACTCCCTGGGCAACTACTACGCCACTTGGTACCAGCAGAAGCCGGG |
| | | CCAAGCCCCTGTGTTGGTCATCTACGGGACCAACAACAGACCTTCCGGC |
| | | ATCCCCGACCGGTTCAGCGCTTCGTCCTCCGGCAACACTGCCAGCCTGA |
| | | CCATCACTGGAGCGCAGGCCGAAGATGAGGCCGACTACTACTGCAACAG |
| | | CAGAGACTCCTCGGGTCATCACCTCTTGTTCGGAACTGGAACCAAGGTC |
| | | ACCGTGCTG |
| 149369-aa | 178 | EVQLQQSGPGLVKPSQTLSLTCAISGDSVSSNSAAWNWIRQSPSRGLEW |
| VH | | LGRTYYRSKWYSFYAISLKSRIIINPDTSKNQFSLQLKSVTPEDTAVYY |
| | | CARSSPEGLFLYWFDPWGQGTLVTVSS |
| 149369-aa VL | 199 | SSELTQDPAVSVALGQTIRITCQGDSLGNYYATWYQQKPGQAPVLVIYG |
| | | TNNRPSGIPDRFSASSSGNTASLTITGAQAEDEADYYCNSRDSSGHHLL |
| | | FGTGTKVTVL |
| 149369-aa | 220 | MALPVTALLLPLALLLHAARPEVQLQQSGPGLVKPSQTLSLTCAISGDS |
| Full CAR | | VSSNSAAWNWIRQSPSRGLEWLGRTYYRSKWYSFYAISLKSRIIINPDT |
| | | SKNQFSLQLKSVTPEDTAVYYCARSSPEGLFLYWFDPWGQGTLVTVSSG |
| | | GDGSGGGGSGGGGSSSELTQDPAVSVALGQTIRITCQGDSLGNYYATWY |
| | | QQKPGQAPVLVIYGTNNRPSGIPDRFSASSSGNTASLTITGAQAEDEAD |
| | | YYCNSRDSSGHHLLFGTGTKVTVLTTTPAPRPPTPAPTIASQPLSLRPE |
| | | ACRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPA |
| | | YKQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALP |
| | | PR |
| 149369-nt | 241 | ATGGCCCTCCCTGTCACCGCCCTGCTGCTTCCGCTGGCTCTTCTGCTCC |
| Full CAR | | ACGCCGCTCGGCCCGAAGTGCAGCTCCAACAGTCAGGACCGGGGCTCGT |
| | | GAAGCCATCCCAGACCCTGTCCCTGACTTGTGCCATCTCGGGAGATAGC |
| | | GTGTCATCGAACTCCGCCGCCTGGAACTGGATTCGGCAGAGCCCGTCCC |
| | | GCGGACTGGAGTGGCTTGGAAGGACCTACTACCGGTCCAAGTGGTACTC |
| | | TTTCTACGCGATCTCGCTGAAGTCCCGCATTATCATTAACCCTGATACC |
| | | TCCAAGAATCAGTTCTCCCTCCAACTGAAATCCGTCACCCCCGAGGACA |
| | | CAGCAGTGTATTACTGCGCACGGAGCAGCCCCGAAGGACTGTTCCTGTA |
| | | TTGGTTTGACCCCTGGGGCCAGGGGACTCTTGTGACCGTGTCGAGCGGC |
| | | GGAGATGGGTCCGGTGGCGGTGGTTCGGGGGGCGGCGGATCATCATCCG |
| | | AACTGACCCAGGACCCGGCTGTGTCCGTGGCGCTGGGACAAACCATCCG |
| | | CATTACGTGCCAGGGAGACTCCCTGGGCAACTACTACGCCACTTGGTAC |
| | | CAGCAGAAGCCGGGCCAAGCCCCTGTGTTGGTCATCTACGGGACCAACA |
| | | ACAGACCTTCCGGCATCCCCGACCGGTTCAGCGCTTCGTCCTCCGGCAA |
| | | CACTGCCAGCCTGACCATCACTGGAGCGCAGGCCGAAGATGAGGCCGAC |
| | | TACTACTGCAACAGCAGAGACTCCTCGGGTCATCACCTCTTGTTCGGAA |
| | | CTGGAACCAAGGTCACCGTGCTGACCACTACCCCAGCACCGAGGCCACC |
| | | CACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACT |
| | | TCGCCTGCGATATCTACATTTGGGCCCCTCTGGCTGGTACTTGCGGGGT |
| | | CCTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACTA |
| | | CTCAAGAGGAGGACGGCTGTTCATGCCGGTTCCCAGAGGAGGAGGAAGG |
| | | CGGCTGCGAACTGCGCGTGAAATTCAGCCGCAGCGCAGATGCTCCAGCC |
| | | TACAAGCAGGGGCAGAACCAGCTCTACAACGAACTCAATCTTGGTCGGA |
| | | GAGAGGAGTACGACGTGCTGGACAAGCGGAGAGGACGGGACCCAGAAAT |
| | | GGGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAAG |
| | | GGGAACGCAGAAGAGGCAAAGGCCACGACGGACTGTACCAGGGACTCAG |
| | | CACCGCCACCAAGGACACCTATGACGCTCTTCACATGCAGGCCCTGCCG |
| | | CCTCGG |

BCMA_EBB-C1978-A4

| | | |
|---|---|---|
| BCMA_EBB- | 137 | EVQLVESGGGLVQPGGSLRLSCAASGFTFSSYAMSWVRQAPGKGLEWVS |
| C1978-A4- | | AISGSGGSTYYADSVKGRFTISRDNSKNTLYLQMNSLRAEDTAVYYCAK |
| aa | | VEGSGSLDYWGQGTLVTVSSGGGGSGGGGSGGGGSEIVMTQSPGTLSLS |
| ScFv domain | | PGERATLSCRASQSVSSAYLAWYQQKPGQPPRLLISGASTRATGIPDRF |
| | | GGSGSGTDFTLTISRLEPEDFAVYYCQHYGSSFNGSSLFTFGQGTRLEI |
| | | K |
| BCMA_EBB- | 158 | GAAGTGCAGCTCGTGGAGTCAGGAGGCGGCCTGGTCCAGCCGGGAGGGT |
| C1978-A4-nt | | CCCTTAGACTGTCATGCGCCGCAAGCGGATTCACTTTCTCCTCCTATGC |
| ScFv domain | | CATGAGCTGGGTCCGCCAAGCCCCCGGAAAGGGACTGGAATGGGTGTCC |
| | | GCCATCTCGGGGTCTGGAGGCTCAACTTACTACGCTGACTCCGTGAAGG |
| | | GACGGTTCACCATTAGCCGCGACAACTCCAAGAACACCCTCTACCTCCA |
| | | AATGAACTCCCTGCGGGCCGAGGATACCGCCGTCTACTACTGCGCCAAA |
| | | GTGGAAGGTTCAGGATCGCTGGACTACTGGGGACAGGGTACTCTCGTGA |
| | | CCGTGTCATCGGGCGGAGGAGGTTCCGGCGGTGGCGGCTCCGGCGGCGG |
| | | AGGGTCGGAGATCGTGATGACCCAGAGCCCTGGTACTCTGAGCCTTTCG |
| | | CCGGGAGAAAGGGCCACCCTGTCCTGCCGCGCTTCCCAATCCGTGTCCT |
| | | CCGCGTACTTGGCGTGGTACCAGCAGAAGCCGGGACAGCCCCCTCGGCT |
| | | GCTGATCAGCGGGGCCAGCACCCGGGCAACCGGAATCCCAGACAGATTC |
| | | GGGGGTTCCGGCAGCGGCACAGATTTCACCCTGACTATTTCGAGGTTGG |
| | | AGCCCGAGGACTTTGCGGTGTATTACTGTCAGCACTACGGGTCGTCCTT |
| | | TAATGGCTCCAGCCTGTTCACGTTCGGACAGGGGACCCGCCTGGAAATC |
| | | AAG |
| BCMA_EBB- | 179 | EVQLVESGGGLVQPGGSLRLSCAASGFTFSSYAMSWVRQAPGKGLEWVS |
| C1978-A4- | | AISGSGGSTYYADSVKGRFTISRDNSKNTLYLQMNSLRAEDTAVYYCAK |
| aa | | VEGSGSLDYWGQGTLVTVSS |
| VH | | |
| BCMA_EBB- | 200 | EIVMTQSPGTLSLSPGERATLSCRASQSVSSAYLAWYQQKPGQPPRLLI |
| C1978-A4- | | SGASTRATGIPDRFGGSGSGTDFTLTISRLEPEDFAVYYCQHYGSSFNG |
| aa | | SSLFTFGQGTRLEIK |
| VL | | |
| BCMA_EBB- | 221 | MALPVTALLLPLALLLHAARPEVQLVESGGGLVQPGGSLRLSCAASGFT |
| C1978-A4- | | FSSYAMSWVRQAPGKGLEWVSAISGSGGSTYYADSVKGRFTISRDNSKN |
| aa | | TLYLQMNSLRAEDTAVYYCAKVEGSGSLDYWGQGTLVTVSSGGGGSGGG |
| Full CART | | GSGGGGSEIVMTQSPGTLSLSPGERATLSCRASQSVSSAYLAWYQQKPG |
| | | QPPRLLISGASTRATGIPDRFGGSGSGTDFTLTISRLEPEDFAVYYCQH |
| | | YGSSFNGSSLFTFGQGTRLEIKTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| BCMA_EBB- | 242 | ATGGCCCTCCCTGTCACCGCCCTGCTGCTTCCGCTGGCTCTTCTGCTCC |
| C1978-A4-nt | | ACGCCGCTCGGCCCGAAGTGCAGCTCGTGGAGTCAGGAGGCGGCCTGGT |
| Full CART | | CCAGCCGGGAGGGTCCCTTAGACTGTCATGCGCCGCAAGCGGATTCACT |
| | | TTCTCCTCCTATGCCATGAGCTGGGTCCGCCAAGCCCCCGGAAAGGGAC |
| | | TGGAATGGGTGTCCGCCATCTCGGGGTCTGGAGGCTCAACTTACTACGC |
| | | TGACTCCGTGAAGGGACGGTTCACCATTAGCCGCGACAACTCCAAGAAC |
| | | ACCCTCTACCTCCAAATGAACTCCCTGCGGGCCGAGGATACCGCCGTCT |
| | | ACTACTGCGCCAAAGTGGAAGGTTCAGGATCGCTGGACTACTGGGGACA |
| | | GGGTACTCTCGTGACCGTGTCATCGGGCGGAGGAGGTTCCGGCGGTGGC |
| | | GGCTCCGGCGGCGGAGGGTCGGAGATCGTGATGACCCAGAGCCCTGGTA |
| | | CTCTGAGCCTTTCGCCGGGAGAAAGGGCCACCCTGTCCTGCCGCGCTTC |
| | | CCAATCCGTGTCCTCCGCGTACTTGGCGTGGTACCAGCAGAAGCCGGGA |
| | | CAGCCCCCTCGGCTGCTGATCAGCGGGGCCAGCACCCGGGCAACCGGAA |
| | | TCCCAGACAGATTCGGGGGTTCCGGCAGCGGCACAGATTTCACCCTGAC |
| | | TATTTCGAGGTTGGAGCCCGAGGACTTTGCGGTGTATTACTGTCAGCAC |
| | | TACGGGTCGTCCTTTAATGGCTCCAGCCTGTTCACGTTCGGACAGGGGA |
| | | CCCGCCTGGAAATCAAGACCACTACCCCAGCACCGAGGCCACCCACCCC |
| | | GGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAG |
| | | AGGAGGACGGCTGTTCATGCCGGTTCCCAGAGGAGGAGGAAGGCGGCTG |
| | | CGAACTGCGCGTGAAATTCAGCCGCAGCGCAGATGCTCCAGCCTACAAG |
| | | CAGGGGCAGAACCAGCTCTACAACGAACTCAATCTTGGTCGGAGAGAGG |
| | | AGTACGACGTGCTGGACAAGCGGAGAGGACGGGACCCAGAAATGGGCGG |
| | | GAAGCCGCGCAGAAAGAATCCCCAAGAGGGCCTGTACAACGAGCTCCAA |
| | | AAGGATAAGATGGCAGAAGCCTATAGCGAGATTGGTATGAAAGGGGAAC |
| | | GCAGAAGAGGCAAAGGCCACGACGGACTGTACCAGGGACTCAGCACCGC |
| | | CACCAAGGACACCTATGACGCTCTTCACATGCAGGCCCTGCCGCCTCGG |

BCMA_EBB-C1978-G1

| | | |
|---|---|---|
| BCMA_EBB- | 138 | EVQLVETGGGLVQPGGSLRLSCAASGITFSRYPMSWVRQAPGKGL |
| C1978-G1- | | EWVSGISDSGVSTYYADSAKGRFTISRDNSKNTLFLQMSSLRDEDT |
| aa | | AVYYCVTRAGSEASDIWGQGTMVTVSSGGGGSGGGGSGGGGSEI |
| ScFv domain | | VLTQSPATLSLSPGERATLSCRASQSVSNSLAWYQQKPGQAPRLLI |
| | | YDASSRATGIPDRFSGSGSGTDFTLTISRLEPEDFAIYYCQQFGTSSG |
| | | LTFGGGTKLEIK |
| BCMA_EBB- | 159 | GAAGTGCAACTGGTGGAAACCGGTGGCGGCCTGGTGCAGCCTGGAGGAT |
| C1978-G1- | | CATTGAGGCTGTCATGCGCGGCCAGCGGTATTACCTTCTCCCGGTACCC |
| nt | | CATGTCCTGGGTCAGACAGGCCCCGGGGAAAGGGCTTGAATGGGTGTCC |
| ScFv domain | | GGGATCTCGGACTCCGGTGTCAGCACTTACTACGCCGACTCCGCCAAGG |
| | | GACGCTTCACCATTTCCCGGGACAACTCGAAGAACACCCTGTTCCTCCA |
| | | AATGAGCTCCCTCCGGGACGAGGATACTGCAGTGTACTACTGCGTGACC |
| | | CGCGCCGGGTCCGAGGCGTCTGACATTTGGGGACAGGGCACTATGGTCA |
| | | CCGTGTCGTCCGGCGGAGGGGGCTCGGGAGGCGGTGGCAGCGGAGGAGG |
| | | AGGGTCCGAGATCGTGCTGACCCAATCCCCGGCCACCCTCTCGCTGAGC |
| | | CCTGGAGAAAGGGCAACCTTGTCCTGTCGCGCGAGCCAGTCCGTGAGCA |
| | | ACTCCCTGGCCTGGTACCAGCAGAAGCCCGGACAGGCTCCGAGACTTCT |
| | | GATCTACGACGCTTCGAGCCGGGCCACTGGAATCCCCGACCGCTTTTCG |
| | | GGGTCCGGCTCAGGAACCGATTTCACCCTGACAATCTCACGGCTGGAGC |
| | | CAGAGGATTTCGCCATCTATTACTGCCAGCAGTTCGGTACTTCCTCCGG |
| | | CCTGACTTTCGGAGGCGGCACGAAGCTCGAAATCAAG |
| BCMA_EBB- | 180 | EVQLVETGGGLVQPGGSLRLSCAASGITFSRYPMSWVRQAPGKGLEWVS |
| C1978-G1- | | GISDSGVSTYYADSAKGRFTISRDNSKNTLFLQMSSLRDEDTAVYYCVT |
| aa | | RAGSEASDIWGQGTMVTVSS |
| VH | | |
| BCMA_EBB- | 201 | EIVLTQSPATLSLSPGERATLSCRASQSVSNSLAWYQQKPGQAPRLLIY |
| C1978-G1- | | DASSRATGIPDRFSGSGSGTDFTLTISRLEPEDFAIYYCQQFGTSSGLT |
| aa | | FGGGTKLEIK |
| VL | | |
| BCMA_EBB- | 222 | MALPVTALLLPLALLLHAARPEVQLVETGGGLVQPGGSLRLSCAA |
| C1978-G1- | | SGITFSRYPMSWVRQAPGKGLEWVSGISDSGVSTYYADSAKGRFTI |
| aa | | SRDNSKNTLFLQMSSLRDEDTAVYYCVTRAGSEASDIWGQGTMV |
| Full CART | | TVSSGGGGSGGGGSGGGGSEIVLTQSPATLSLSPGERATLSCRASQ |
| | | SVSNSLAWYQQKPGQAPRLLIYDASSRATGIPDRFSGSGSGTDFTL |
| | | TISRLEPEDFAIYYCQQFGTSSGLTFGGGTKLEIKTTTPAPRPPTPAP |
| | | TIASQPLSLRPEACRPAAGGAVHTRGLDFACDIYIWAPLAGTCGVL |
| | | LLSLVITLYCKRGRKKLLYIFKQPFMRPVQTTQEEDGCSCRFPEEEE |
| | | GGCELRVKFSRSADAPAYKQGQNQLYNELNLGRREEYDVLDKRR |
| | | GRDPEMGGKPRRKNPQEGLYNELQKDKMAEAYSEIGMKGERRRG |
| | | KGHDGLYQGLSTATKDTYDALHMQALPPR |
| BCMA_EBB- | 243 | ATGGCCCTCCCTGTCACCGCCCTGCTGCTTCCGCTGGCTCTTCTGCTCC |
| C1978-G1- | | ACGCCGCTCGGCCCGAAGTGCAACTGGTGGAAACCGGTGGCGGCCTGGT |
| nt | | GCAGCCTGGAGGATCATTGAGGCTGTCATGCGCGGCCAGCGGTATTACC |
| Full CART | | TTCTCCCGGTACCCCATGTCCTGGGTCAGACAGGCCCCGGGGAAAGGGC |
| | | TTGAATGGGTGTCCGGGATCTCGGACTCCGGTGTCAGCACTTACTACGC |
| | | CGACTCCGCCAAGGGACGCTTCACCATTTCCCGGGACAACTCGAAGAAC |
| | | ACCCTGTTCCTCCAAATGAGCTCCCTCCGGGACGAGGATACTGCAGTGT |
| | | ACTACTGCGTGACCCGCGCCGGGTCCGAGGCGTCTGACATTTGGGGACA |
| | | GGGCACTATGGTCACCGTGTCGTCCGGCGGAGGGGGCTCGGGAGGCGGT |
| | | GGCAGCGGAGGAGGAGGGTCCGAGATCGTGCTGACCCAATCCCCGGCCA |
| | | CCCTCTCGCTGAGCCCTGGAGAAAGGGCAACCTTGTCCTGTCGCGCGAG |
| | | CCAGTCCGTGAGCAACTCCCTGGCCTGGTACCAGCAGAAGCCCGGACAG |
| | | GCTCCGAGACTTCTGATCTACGACGCTTCGAGCCGGGCCACTGGAATCC |
| | | CCGACCGCTTTTCGGGGTCCGGCTCAGGAACCGATTTCACCCTGACAAT |
| | | CTCACGGCTGGAGCCAGAGGATTTCGCCATCTATTACTGCCAGCAGTTC |
| | | GGTACTTCCTCCGGCCTGACTTTCGGAGGCGGCACGAAGCTCGAAATCA |
| | | AGACCACTACCCCAGCACCGAGGCCACCCACCCCGGCTCCTACCATCGC |
| | | CTCCCAGCCTCTGTCCCTGCGTCCGGAGGCATGTAGACCCGCAGCTGGT |
| | | GGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTT |
| | | CATGCCGGTTCCCAGAGGAGGAGGAAGGCGGCTGCGAACTGCGCGTGAA |
| | | ATTCAGCCGCAGCGCAGATGCTCCAGCCTACAAGCAGGGGCAGAACCAG |
| | | CTCTACAACGAACTCAATCTTGGTCGGAGAGAGGAGTACGACGTGCTGG |
| | | ACAAGCGGAGAGGACGGGACCCAGAAATGGGCGGGAAGCCGCGCAGAAA |
| | | GAATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAAG |
| | | GCCACGACGGACTGTACCAGGGACTCAGCACCGCCACCAAGGACACCTA |
| | | TGACGCTCTTCACATGCAGGCCCTGCCGCCTCGG |

BCMA_EBB-C1979-C1

| | | |
|---|---|---|
| BCMA_EBB- | 139 | QVQLVESGGGLVQPGGSLRLSCAASGFTFSSYAMSWVRQAPGKGLEWVS |
| C1979-C1- | | AISGSGGSTYYADSVKGRFTISRDNAKNSLYLQMNSLRAEDTAIYYCAR |
| aa | | ATYKRELRYYYGMDVWGQGTMVTVSSGGGGSGGGGSGGGGSEIVMTQSP |
| ScFv domain | | GTVSLSPGERATLSCRASQSVSSSFLAWYQQKPGQAPRLLIYGASSRAT |
| | | GIPDRFSGSGSGTDFTLTISRLEPEDSAVYYCQQYHSSPSWTFGQGTRL |
| | | EIK |
| BCMA_EBB- | 160 | CAAGTGCAGCTCGTGGAATCGGGTGGCGGACTGGTGCAGCCGGGGGGCT |
| C1979-C1-nt | | CACTTAGACTGTCCTGCGCGGCCAGCGGATTCACTTTCTCCTCCTACGC |
| ScFv domain | | CATGTCCTGGGTCAGACAGGCCCCTGGAAAGGGCCTGGAATGGGTGTCC |
| | | GCAATCAGCGGCAGCGGCGGCTCGACCTATTACGCGGATTCAGTGAAGG |
| | | GCAGATTCACCATTTCCCGGGACAACGCCAAGAACTCCTTGTACCTTCA |
| | | AATGAACTCCCTCCGCGCGGAAGATACCGCAATCTACTACTGCGCTCGG |
| | | GCCACTTACAAGAGGGAACTGCGCTACTACTACGGGATGGACGTCTGGG |
| | | GCCAGGGAACCATGGTCACCGTGTCCAGCGGAGGAGGAGGATCGGGAGG |
| | | AGGCGGTAGCGGGGGTGGAGGGTCGGAGATCGTGATGACCCAGTCCCCC |
| | | GGCACTGTGTCGCTGTCCCCCGGCGAACGGGCCACCCTGTCATGTCGGG |
| | | CCAGCCAGTCAGTGTCGTCAAGCTTCCTCGCCTGGTACCAGCAGAAACC |
| | | GGGACAAGCTCCCCGCCTGCTGATCTACGGAGCCAGCAGCCGGGCCACC |
| | | GGTATTCCTGACCGGTTCTCCGGTTCGGGGTCCGGGACCGACTTTACTC |
| | | TGACTATCTCTCGCCTCGAGCCAGAGGACTCCGCCGTGTATTACTGCCA |
| | | GCAGTACCACTCCTCCCCGTCCTGGACGTTCGGACAGGGCACAAGGCTG |
| | | GAGATTAAG |
| BCMA_EBB- | 181 | QVQLVESGGGLVQPGGSLRLSCAASGFTFSSYAMSWVRQAPGKGLEWVS |
| C1979-C1- | | AISGSGGSTYYADSVKGRFTISRDNAKNSLYLQMNSLRAEDTAIYYCAR |
| aa | | ATYKRELRYYYGMDVWGQGTMVTVSS |
| VH | | |
| BCMA_EBB- | 202 | EIVMTQSPGTVSLSPGERATLSCRASQSVSSSFLAWYQQKPGQAPRLLI |
| C1979-C1- | | YGASSRATGIPDRFSGSGSGTDFTLTISRLEPEDSAVYYCQQYHSSPSW |
| aa | | TFGQGTRLEIK |
| VL | | |
| BCMA_EBB- | 223 | MALPVTALLLPLALLLHAARPQVQLVESGGGLVQPGGSLRLSCAASGFT |
| C1979-C1- | | FSSYAMSWVRQAPGKGLEWVSAISGSGGSTYYADSVKGRFTISRDNAKN |
| aa | | SLYLQMNSLRAEDTAIYYCARATYKRELRYYYGMDVWGQGTMVTVSSGG |
| Full CART | | GGSGGGGSGGGGSEIVMTQSPGTVSLSPGERATLSCRASQSVSSSFLAW |
| | | YQQKPGQAPRLLIYGASSRATGIPDRFSGSGSGTDFTLTISRLEPEDSA |
| | | VYYCQQYHSSPSWTFGQGTRLEIKTTTPAPRPPTPAPTIASQPLSLRPE |
| | | ACRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPA |
| | | YKQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALP |
| | | PR |
| BCMA_EBB- | 244 | ATGGCCCTCCCTGTCACCGCCCTGCTGCTTCCGCTGGCTCTTCTGCTCC |
| C1979-C1-nt | | ACGCCGCTCGGCCCCAAGTGCAGCTCGTGGAATCGGGTGGCGGACTGGT |
| Full CART | | GCAGCCGGGGGGCTCACTTAGACTGTCCTGCGCGGCCAGCGGATTCACT |
| | | TTCTCCTCCTACGCCATGTCCTGGGTCAGACAGGCCCCTGGAAAGGGCC |
| | | TGGAATGGGTGTCCGCAATCAGCGGCAGCGGCGGCTCGACCTATTACGC |
| | | GGATTCAGTGAAGGGCAGATTCACCATTTCCCGGGACAACGCCAAGAAC |
| | | TCCTTGTACCTTCAAATGAACTCCCTCCGCGCGGAAGATACCGCAATCT |
| | | ACTACTGCGCTCGGGCCACTTACAAGAGGGAACTGCGCTACTACTACGG |
| | | GATGGACGTCTGGGGCCAGGGAACCATGGTCACCGTGTCCAGCGGAGGA |
| | | GGAGGATCGGGAGGAGGCGGTAGCGGGGGTGGAGGGTCGGAGATCGTGA |
| | | TGACCCAGTCCCCCGGCACTGTGTCGCTGTCCCCCGGCGAACGGGCCAC |
| | | CCTGTCATGTCGGGCCAGCCAGTCAGTGTCGTCAAGCTTCCTCGCCTGG |
| | | TACCAGCAGAAACCGGGACAAGCTCCCCGCCTGCTGATCTACGGAGCCA |
| | | GCAGCCGGGCCACCGGTATTCCTGACCGGTTCTCCGGTTCGGGGTCCGG |
| | | GACCGACTTTACTCTGACTATCTCTCGCCTCGAGCCAGAGGACTCCGCC |
| | | GTGTATTACTGCCAGCAGTACCACTCCTCCCCGTCCTGGACGTTCGGAC |
| | | AGGGCACAAGGCTGGAGATTAAGACCACTACCCCAGCACCGAGGCCACC |
| | | CACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACT |
| | | TCGCCTGCGATATCTACATTTGGGCCCCTCTGGCTGGTACTTGCGGGGT |
| | | CCTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACTA |
| | | CTCAAGAGGAGGACGGCTGTTCATGCCGGTTCCCAGAGGAGGAGGAAGG |
| | | CGGCTGCGAACTGCGCGTGAAATTCAGCCGCAGCGCAGATGCTCCAGCC |
| | | TACAAGCAGGGGCAGAACCAGCTCTACAACGAACTCAATCTTGGTCGGA |
| | | GAGAGGAGTACGACGTGCTGGACAAGCGGAGAGGACGGGACCCAGAAAT |
| | | GGGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAAG |
| | | GGGAACGCAGAAGAGGCAAAGGCCACGACGGACTGTACCAGGGACTCAG |
| | | CACCGCCACCAAGGACACCTATGACGCTCTTCACATGCAGGCCCTGCCG |
| | | CCTCGG |

BCMA_EBB-C1978-C7

| | | |
|---|---|---|
| BCMA_EBB- | 140 | EVQLVETGGGLVQPGGSLRLSCAASGFTFSSYAMSWVRQAPGKGLEWVS |
| C1978-C7- | | AISGSGGSTYYADSVKGRFTISRDNSKNTLYLQMNTLKAEDTAVYYCAR |
| aa | | ATYKRELRYYYGMDVWGQGTTVTVSSGGGGSGGGGSGGGGSEIVLTQSP |
| ScFv domain | | STLSLSPGESATLSCRASQSVSTTFLAWYQQKPGQAPRLLIYGSSNRAT |
| | | GIPDRFSGSGSGTDFTLTIRRLEPEDFAVYYCQQYHSSPSWTFGQGTKV |
| | | EIK |
| BCMA_EBB- | 161 | GAGGTGCAGCTTGTGGAAACCGGTGGCGGACTGGTGCAGCCCGGAGGAA |
| C1978-C7-nt | | GCCTCAGGCTGTCCTGCGCCGCGTCCGGCTTCACCTTCTCCTCGTACGC |
| ScFv domain | | CATGTCCTGGGTCCGCCAGGCCCCCGGAAAGGGCCTGGAATGGGTGTCC |
| | | GCCATCTCTGGAAGCGGAGGTTCCACGTACTACGCGGACAGCGTCAAGG |
| | | GAAGGTTCACAATCTCCCGCGATAATTCGAAGAACACTCTGTACCTTCA |
| | | AATGAACACCCTGAAGGCCGAGGACACTGCTGTGTACTACTGCGCACGG |
| | | GCCACCTACAAGAGAGAGCTCCGGTACTACTACGGAATGGACGTCTGGG |
| | | GCCAGGGAACTACTGTGACCGTGTCCTCGGGAGGGGGTGGCTCCGGGGG |
| | | GGGCGGCTCCGGCGGAGGCGGTTCCGAGATTGTGCTGACCCAGTCACCT |
| | | TCAACTCTGTCGCTGTCCCCGGGAGAGAGCGCTACTCTGAGCTGCCGGG |
| | | CCAGCCAGTCCGTGTCCACCACCTTCCTCGCCTGGTATCAGCAGAAGCC |
| | | GGGGCAGGCACCACGGCTCTTGATCTACGGGTCAAGCAACAGAGCGACC |
| | | GGAATTCCTGACCGCTTCTCGGGGAGCGGTTCAGGCACCGACTTCACCC |
| | | TGACTATCCGGCGCCTGGAACCCGAAGATTTCGCCGTGTATTACTGTCA |
| | | ACAGTACCACTCCTCGCCGTCCTGGACCTTTGGCCAAGGAACCAAAGTG |
| | | GAAATCAAG |
| BCMA_EBB- | 182 | EVQLVETGGGLVQPGGSLRLSCAASGFTFSSYAMSWVRQAPGKGLEWVS |
| C1978-C7- | | AISGSGGSTYYADSVKGRFTISRDNSKNTLYLQMNTLKAEDTAVYYCAR |
| aa | | ATYKRELRYYYGMDVWGQGTTVTVSS |
| VH | | |
| BCMA_EBB- | 203 | EIVLTQSPSTLSLSPGESATLSCRASQSVSTTFLAWYQQKPGQAPRLLI |
| C1978-C7- | | YGSSNRATGIPDRFSGSGSGTDFTLTIRRLEPEDFAVYYCQQYHSSPSW |
| aa | | TFGQGTKVEIK |
| VL | | |
| BCMA_EBB- | 224 | MALPVTALLLPLALLLHAARPEVQLVETGGGLVQPGGSLRLSCAASGFT |
| C1978-C7- | | FSSYAMSWVRQAPGKGLEWVSAISGSGGSTYYADSVKGRFTISRDNSKN |
| aa | | TLYLQMNTLKAEDTAVYYCARATYKRELRYYYGMDVWGQGTTVTVSSGG |
| Full CART | | GGSGGGGSGGGGSEIVLTQSPSTLSLSPGESATLSCRASQSVSTTFLAW |
| | | YQQKPGQAPRLLIYGSSNRATGIPDRFSGSGSGTDFTLTIRRLEPEDFA |
| | | VYYCQQYHSSPSWTFGQGTKVEIKTTTPAPRPPTPAPTIASQPLSLRPE |
| | | ACRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPA |
| | | YKQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALP |
| | | PR |
| BCMA_EBB- | 245 | ATGGCCCTCCCTGTCACCGCCCTGCTGCTTCCGCTGGCTCTTCTGCTCC |
| C1978-C7-nt | | ACGCCGCTCGGCCCGAGGTGCAGCTTGTGGAAACCGGTGGCGGACTGGT |
| Full CART | | GCAGCCCGGAGGAAGCCTCAGGCTGTCCTGCGCCGCGTCCGGCTTCACC |
| | | TTCTCCTCGTACGCCATGTCCTGGGTCCGCCAGGCCCCCGGAAAGGGCC |
| | | TGGAATGGGTGTCCGCCATCTCTGGAAGCGGAGGTTCCACGTACTACGC |
| | | GGACAGCGTCAAGGGAAGGTTCACAATCTCCCGCGATAATTCGAAGAAC |
| | | ACTCTGTACCTTCAAATGAACACCCTGAAGGCCGAGGACACTGCTGTGT |
| | | ACTACTGCGCACGGGCCACCTACAAGAGAGAGCTCCGGTACTACTACGG |
| | | AATGGACGTCTGGGGCCAGGGAACTACTGTGACCGTGTCCTCGGGAGGG |
| | | GGTGGCTCCGGGGGGGGCGGCTCCGGCGGAGGCGGTTCCGAGATTGTGC |
| | | TGACCCAGTCACCTTCAACTCTGTCGCTGTCCCCGGGAGAGAGCGCTAC |
| | | TCTGAGCTGCCGGGCCAGCCAGTCCGTGTCCACCACCTTCCTCGCCTGG |
| | | TATCAGCAGAAGCCGGGGCAGGCACCACGGCTCTTGATCTACGGGTCAA |
| | | GCAACAGAGCGACCGGAATTCCTGACCGCTTCTCGGGGAGCGGTTCAGG |
| | | CACCGACTTCACCCTGACTATCCGGCGCCTGGAACCCGAAGATTTCGCC |
| | | GTGTATTACTGTCAACAGTACCACTCCTCGCCGTCCTGGACCTTTGGCC |
| | | AAGGAACCAAAGTGGAAATCAAGACCACTACCCCAGCACCGAGGCCACC |
| | | CACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACT |
| | | TCGCCTGCGATATCTACATTTGGGCCCCTCTGGCTGGTACTTGCGGGGT |
| | | CCTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACTA |
| | | CTCAAGAGGAGGACGGCTGTTCATGCCGGTTCCCAGAGGAGGAGGAAGG |
| | | CGGCTGCGAACTGCGCGTGAAATTCAGCCGCAGCGCAGATGCTCCAGCC |
| | | TACAAGCAGGGGCAGAACCAGCTCTACAACGAACTCAATCTTGGTCGGA |
| | | GAGAGGAGTACGACGTGCTGGACAAGCGGAGAGGACGGGACCCAGAAAT |
| | | GGGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAAG |
| | | GGGAACGCAGAAGAGGCAAAGGCCACGACGGACTGTACCAGGGACTCAG |
| | | CACCGCCACCAAGGACACCTATGACGCTCTTCACATGCAGGCCCTGCCG |
| | | CCTCGG |

BCMA_EBB-C1978-D10

| | | |
|---|---|---|
| BCMA_EBB- | 141 | EVQLVETGGGLVQPGRSLRLSCAASGFTFDDYAMHWVRQAPGKGLEWVS |
| C1978-D10- | | GISWNSGSIGYADSVKGRFTISRDNAKNSLYLQMNSLRDEDTAVYYCAR |
| aa | | VGKAVPDVWGQGTTVTVSSGGGGSGGGGSGGGGSDIVMTQTPSSLSASV |
| ScFv domain | | GDRVTITCRASQSISSYLNWYQQKPGKAPKLLIYAASSLQSGVPSRFSG |
| | | SGSGTDFTLTISSLQPEDFATYYCQQSYSTPYSFGQGTRLEIK |
| BCMA_EBB- | 162 | GAAGTGCAGCTCGTGGAAACTGGAGGTGGACTCGTGCAGCCTGGACGGT |
| C1978-D10- | | CGCTGCGGCTGAGCTGCGCTGCATCCGGCTTCACCTTCGACGATTATGC |
| nt | | CATGCACTGGGTCAGACAGGCGCCAGGGAAGGGACTTGAGTGGGTGTCC |
| ScFv domain | | GGTATCAGCTGGAATAGCGGCTCAATCGGATACGCGGACTCCGTGAAGG |
| | | GAAGGTTCACCATTTCCCGCGACAACGCCAAGAACTCCCTGTACTTGCA |
| | | AATGAACAGCCTCCGGGATGAGGACACTGCCGTGTACTACTGCGCCCGC |
| | | GTCGGAAAAGCTGTGCCCGACGTCTGGGGCCAGGGAACCACTGTGACCG |
| | | TGTCCAGCGGCGGGGGTGGATCGGGCGGTGGAGGGTCCGGTGGAGGGGG |
| | | CTCAGATATTGTGATGACCCAGACCCCCTCGTCCCTGTCCGCCTCGGTC |
| | | GGCGACCGCGTGACTATCACATGTAGAGCCTCGCAGAGCATCTCCAGCT |
| | | ACCTGAACTGGTATCAGCAGAAGCCGGGGAAGGCCCCGAAGCTCCTGAT |
| | | CTACGCGGCATCATCACTGCAATCGGGAGTGCCGAGCCGGTTTTCCGGG |
| | | TCCGGCTCCGGCACCGACTTCACGCTGACCATTTCTTCCCTGCAACCCG |
| | | AGGACTTCGCCACTTACTACTGCCAGCAGTCCTACTCCACCCCTTACTC |
| | | CTTCGGCCAAGGAACCAGGCTGGAAATCAAG |
| BCMA_EBB- | 183 | EVQLVETGGGLVQPGRSLRLSCAASGFTFDDYAMHWVRQAPGKGLEWVS |
| C1978-D10- | | GISWNSGSIGYADSVKGRFTISRDNAKNSLYLQMNSLRDEDTAVYYCAR |
| aa | | VGKAVPDVWGQGTTVTVSS |
| VH | | |
| BCMA_EBB- | 204 | DIVMTQTPSSLSASVGDRVTITCRASQSISSYLNWYQQKPGKAPKLLIY |
| C1978-D10- | | AASSLQSGVPSRFSGSGSGTDFTLTISSLQPEDFATYYCQQSYSTPYSF |
| aa | | GQGTRLEIK |
| VL | | |
| BCMA_EBB- | 225 | MALPVTALLLPLALLLHAARPEVQLVETGGGLVQPGRSLRLSCAASGFT |
| C1978-D10- | | FDDYAMHWVRQAPGKGLEWVSGISWNSGSIGYADSVKGRFTISRDNAKN |
| aa | | SLYLQMNSLRDEDTAVYYCARVGKAVPDVWGQGTTVTVSSGGGGSGGGG |
| Full CART | | SGGGGSDIVMTQTPSSLSASVGDRVTITCRASQSISSYLNWYQQKPGKA |
| | | PKLLIYAASSLQSGVPSRFSGSGSGTDFTLTISSLQPEDFATYYCQQSY |
| | | STPYSFGQGTRLEIKTTTPAPRPPTPAPTIASQPLSLRPEACRPAAGGA |
| | | VHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| BCMA_EBB- | 246 | ATGGCCCTCCCTGTCACCGCCCTGCTGCTTCCGCTGGCTCTTCTGCTCC |
| C1978-D10- | | ACGCCGCTCGGCCCGAAGTGCAGCTCGTGGAAACTGGAGGTGGACTCGT |
| nt | | GCAGCCTGGACGGTCGCTGCGGCTGAGCTGCGCTGCATCCGGCTTCACC |
| Full CART | | TTCGACGATTATGCCATGCACTGGGTCAGACAGGCGCCAGGGAAGGGAC |
| | | TTGAGTGGGTGTCCGGTATCAGCTGGAATAGCGGCTCAATCGGATACGC |
| | | GGACTCCGTGAAGGGAAGGTTCACCATTTCCCGCGACAACGCCAAGAAC |
| | | TCCCTGTACTTGCAAATGAACAGCCTCCGGGATGAGGACACTGCCGTGT |
| | | ACTACTGCGCCCGCGTCGGAAAAGCTGTGCCCGACGTCTGGGGCCAGGG |
| | | AACCACTGTGACCGTGTCCAGCGGCGGGGGTGGATCGGGCGGTGGAGGG |
| | | TCCGGTGGAGGGGGCTCAGATATTGTGATGACCCAGACCCCCTCGTCCC |
| | | TGTCCGCCTCGGTCGGCGACCGCGTGACTATCACATGTAGAGCCTCGCA |
| | | GAGCATCTCCAGCTACCTGAACTGGTATCAGCAGAAGCCGGGGAAGGCC |
| | | CCGAAGCTCCTGATCTACGCGGCATCATCACTGCAATCGGGAGTGCCGA |
| | | GCCGGTTTTCCGGGTCCGGCTCCGGCACCGACTTCACGCTGACCATTTC |
| | | TTCCCTGCAACCCGAGGACTTCGCCACTTACTACTGCCAGCAGTCCTAC |
| | | TCCACCCCTTACTCCTTCGGCCAAGGAACCAGGCTGGAAATCAAGACCA |
| | | CTACCCCAGCACCGAGGCCACCCACCCCGGCTCCTACCATCGCCTCCCA |
| | | GCCTCTGTCCCTGCGTCCGGAGGCATGTAGACCCGCAGCTGGTGGGGCC |
| | | GTGCATACCCGGGGTCTTGACTTCGCCTGCGATATCTACATTTGGGCCC |
| | | CTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCC |
| | | GGTTCCCAGAGGAGGAGGAAGGCGGCTGCGAACTGCGCGTGAAATTCAG |
| | | CCGCAGCGCAGATGCTCCAGCCTACAAGCAGGGGCAGAACCAGCTCTAC |
| | | AACGAACTCAATCTTGGTCGGAGAGAGGAGTACGACGTGCTGGACAAGC |
| | | GGAGAGGACGGGACCCAGAAATGGGCGGGAAGCCGCGCAGAAAGAATCC |
| | | CCAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCACG |
| | | ACGGACTGTACCAGGGACTCAGCACCGCCACCAAGGACACCTATGACGC |
| | | TCTTCACATGCAGGCCCTGCCGCCTCGG |

BCMA_EBB-C1979-C12

| | | |
|---|---|---|
| BCMA_EBB- | 142 | EVQLVESGGGLVQPGRSLRLSCTASGFTFDDYAMHWVRQRPGKGLEWVA |
| C1979-C12- | | SINWKGNSLAYGDSVKGRFAISRDNAKNTVFLQMNSLRTEDTAVYYCAS |
| aa | | HQGVAYYNYAMDVWGRGTLVTVSSGGGGSGGGGSGGGGSEIVLTQSPGT |
| ScFv domain | | LSLSPGERATLSCRATQSIGSSFLAWYQQRPGQAPRLLIYGASQRATGI |
| | | PDRFSGRGSGTDFTLTISRVEPEDSAVYYCQHYESSPSWTFGQGTKVEI |
| | | K |
| BCMA_EBB- | 163 | GAAGTGCAGCTCGTGGAGAGCGGGGGAGGATTGGTGCAGCCCGGAAGGT |
| C1979-C12- | | CCCTGCGGCTCTCCTGCACTGCGTCTGGCTTCACCTTCGACGACTACGC |
| nt | | GATGCACTGGGTCAGACAGCGCCCGGGAAAGGGCCTGGAATGGGTCGCC |
| ScFv domain | | TCAATCAACTGGAAGGGAAACTCCCTGGCCTATGGCGACAGCGTGAAGG |
| | | GCCGCTTCGCCATTTCGCGCGACAACGCCAAGAACACCGTGTTTCTGCA |
| | | AATGAATTCCCTGCGGACCGAGGATACCGCTGTGTACTACTGCGCCAGC |
| | | CACCAGGGCGTGGCATACTATAACTACGCCATGGACGTGTGGGGAAGAG |
| | | GGACGCTCGTCACCGTGTCCTCCGGGGGCGGTGGATCGGGTGGAGGAGG |
| | | AAGCGGTGGCGGGGGCAGCGAAATCGTGCTGACTCAGAGCCCGGGAACT |
| | | CTTTCACTGTCCCCGGGAGAACGGGCCACTCTCTCGTGCCGGGCCACCC |
| | | AGTCCATCGGCTCCTCCTTCCTTGCCTGGTACCAGCAGAGGCCAGGACA |
| | | GGCGCCCCGCCTGCTGATCTACGGTGCTTCCCAACGCGCCACTGGCATT |
| | | CCTGACCGGTTCAGCGGCAGAGGGTCGGGAACCGATTTCACACTGACCA |
| | | TTTCCCGGGTGGAGCCCGAAGATTCGGCAGTCTACTACTGTCAGCATTA |
| | | CGAGTCCTCCCCTTCATGGACCTTCGGTCAAGGGACCAAAGTGGAGATC |
| | | AAG |
| BCMA_EBB- | 184 | EVQLVESGGGLVQPGRSLRLSCTASGFTFDDYAMHWVRQRPGKGLEWVA |
| C1979-C12- | | SINWKGNSLAYGDSVKGRFAISRDNAKNTVFLQMNSLRTEDTAVYYCAS |
| aa | | HQGVAYYNYAMDVWGRGTLVTVSS |
| VH | | |
| BCMA_EBB- | 205 | EIVLTQSPGTLSLSPGERATLSCRATQSIGSSFLAWYQQRPGQAPRLLI |
| C1979-C12- | | YGASQRATGIPDRFSGRGSGTDFTLTISRVEPEDSAVYYCQHYESSPSW |
| aa | | TFGQGTKVEIK |
| VL | | |
| BCMA_EBB- | 226 | MALPVTALLLPLALLLHAARPEVQLVESGGGLVQPGRSLRLSCTASGFT |
| C1979-C12- | | FDDYAMHWVRQRPGKGLEWVASINWKGNSLAYGDSVKGRFAISRDNAKN |
| aa | | TVFLQMNSLRTEDTAVYYCASHQGVAYYNYAMDVWGRGTLVTVSSGGGG |
| Full CART | | SGGGGSGGGGSEIVLTQSPGTLSLSPGERATLSCRATQSIGSSFLAWYQ |
| | | QRPGQAPRLLIYGASQRATGIPDRFSGRGSGTDFTLTISRVEPEDSAVY |
| | | YCQHYESSPSWTFGQGTKVEIKTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| BCMA_EBB- | 247 | ATGGCCCTCCCTGTCACCGCCCTGCTGCTTCCGCTGGCTCTTCTGCTCC |
| C1979-C12- | | ACGCCGCTCGGCCCGAAGTGCAGCTCGTGGAGAGCGGGGGAGGATTGGT |
| nt | | GCAGCCCGGAAGGTCCCTGCGGCTCTCCTGCACTGCGTCTGGCTTCACC |
| Full CART | | TTCGACGACTACGCGATGCACTGGGTCAGACAGCGCCCGGGAAAGGGCC |
| | | TGGAATGGGTCGCCTCAATCAACTGGAAGGGAAACTCCCTGGCCTATGG |
| | | CGACAGCGTGAAGGGCCGCTTCGCCATTTCGCGCGACAACGCCAAGAAC |
| | | ACCGTGTTTCTGCAAATGAATTCCCTGCGGACCGAGGATACCGCTGTGT |
| | | ACTACTGCGCCAGCCACCAGGGCGTGGCATACTATAACTACGCCATGGA |
| | | CGTGTGGGGAAGAGGGACGCTCGTCACCGTGTCCTCCGGGGGCGGTGGA |
| | | TCGGGTGGAGGAGGAAGCGGTGGCGGGGGCAGCGAAATCGTGCTGACTC |
| | | AGAGCCCGGGAACTCTTTCACTGTCCCCGGGAGAACGGGCCACTCTCTC |
| | | GTGCCGGGCCACCCAGTCCATCGGCTCCTCCTTCCTTGCCTGGTACCAG |
| | | CAGAGGCCAGGACAGGCGCCCCGCCTGCTGATCTACGGTGCTTCCCAAC |
| | | GCGCCACTGGCATTCCTGACCGGTTCAGCGGCAGAGGGTCGGGAACCGA |
| | | TTTCACACTGACCATTTCCCGGGTGGAGCCCGAAGATTCGGCAGTCTAC |
| | | TACTGTCAGCATTACGAGTCCTCCCCTTCATGGACCTTCGGTCAAGGGA |
| | | CCAAAGTGGAGATCAAGACCACTACCCCAGCACCGAGGCCACCCACCCC |
| | | GGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAG |
| | | AGGAGGACGGCTGTTCATGCCGGTTCCCAGAGGAGGAGGAAGGCGGCTG |
| | | CGAACTGCGCGTGAAATTCAGCCGCAGCGCAGATGCTCCAGCCTACAAG |
| | | CAGGGGCAGAACCAGCTCTACAACGAACTCAATCTTGGTCGGAGAGAGG |
| | | AGTACGACGTGCTGGACAAGCGGAGAGGACGGGACCCAGAAATGGGCGG |
| | | GAAGCCGCGCAGAAAGAATCCCCAAGAGGGCCTGTACAACGAGCTCCAA |
| | | AAGGATAAGATGGCAGAAGCCTATAGCGAGATTGGTATGAAAGGGGAAC |
| | | GCAGAAGAGGCAAAGGCCACGACGGACTGTACCAGGGACTCAGCACCGC |
| | | CACCAAGGACACCTATGACGCTCTTCACATGCAGGCCCTGCCGCCTCGG |

BCMA_EBB-C1980-G4

| | | |
|---|---|---|
| BCMA_EBB- | 143 | EVQLVESGGGLVQPGGSLRLSCAASGFTFSSYAMSWVRQAPGKGL |
| C1980-G4-aa | | EWVSAISGSGGSTYYADSVKGRFTISRDNSKNTLYLQMNSLRAED |
| ScFv domain | | TAVYYCAKVVRDGMDVWGQGTTVTVSSGGGGSGGGGSGGGGSE |
| | | IVLTQSPATLSLSPGERATLSCRASQSVSSSYLAWYQQKPGQAPRL |
| | | LIYGASSRATGIPDRFSGNGSGTDFTLTISRLEPEDFAVYYCQQYGS |
| | | PPRFTFGPGTKVDIK |
| BCMA_EBB- | 164 | GAGGTGCAGTTGGTCGAAAGCGGGGGCGGGCTTGTGCAGCCTGGCGGAT |
| C1980-G4-nt | | CACTGCGGCTGTCCTGCGCGGCATCAGGCTTCACGTTTTCTTCCTACGC |
| ScFv domain | | CATGTCCTGGGTGCGCCAGGCCCCTGGAAAGGGACTGGAATGGGTGTCC |
| | | GCGATTTCGGGGTCCGGCGGGAGCACCTACTACGCCGATTCCGTGAAGG |
| | | GCCGCTTCACTATCTCGCGGGACAACTCCAAGAACACCCTCTACCTCCA |
| | | AATGAATAGCCTGCGGGCCGAGGATACCGCCGTCTACTATTGCGCTAAG |
| | | GTCGTGCGCGACGGAATGGACGTGTGGGGACAGGGTACCACCGTGACAG |
| | | TGTCCTCGGGGGGAGGCGGTAGCGGCGGAGGAGGAAGCGGTGGTGGAGG |
| | | TTCCGAGATTGTGCTGACTCAATCACCCGCGACCCTGAGCCTGTCCCCC |
| | | GGCGAAAGGGCCACTCTGTCCTGTCGGGCCAGCCAATCAGTCTCCTCCT |
| | | CGTACCTGGCCTGGTACCAGCAGAAGCCAGGACAGGCTCCGAGACTCCT |
| | | TATCTATGGCGCATCCTCCCGCGCCACCGGAATCCCGGATAGGTTCTCG |
| | | GGAAACGGATCGGGGACCGACTTCACTCTCACCATCTCCCGGCTGGAAC |
| | | CGGAGGACTTCGCCGTGTACTACTGCCAGCAGTACGGCAGCCCGCCTAG |
| | | ATTCACTTTCGGCCCCGGCACCAAAGTGGACATCAAG |
| BCMA_EBB- | 185 | EVQLVESGGGLVQPGGSLRLSCAASGFTFSSYAMSWVRQAPGKGLEWVS |
| C1980-G4-aa | | AISGSGGSTYYADSVKGRFTISRDNSKNTLYLQMNSLRAEDTAVYYCAK |
| VH | | VVRDGMDVWGQGTTVTVSS |
| BCMA_EBB- | 206 | EIVLTQSPATLSLSPGERATLSCRASQSVSSSYLAWYQQKPGQAPRLLI |
| C1980-G4-aa | | YGASSRATGIPDRFSGNGSGTDFTLTISRLEPEDFAVYYCQQYGSPPRF |
| VL | | TFGPGTKVDIK |
| BCMA_EBB- | 227 | MALPVTALLLPLALLLHAARPEVQLVESGGGLVQPGGSLRLSCAA |
| C1980-G4-aa | | SGFTFSSYAMSWVRQAPGKGLEWVSAISGSGGSTYYADSVKGRFT |
| Full CART | | ISRDNSKNTLYLQMNSLRAEDTAVYYCAKVVRDGMDVWGQGTT |
| | | VTVSSGGGGSGGGGSGGGGSEIVLTQSPATLSLSPGERATLSCRAS |
| | | QSVSSSYLAWYQQKPGQAPRLLIYGASSRATGIPDRFSGNGSGTDF |
| | | TLTISRLEPEDFAVYYCQQYGSPPRFTFGPGTKVDIKTTTPAPRPPT |
| | | PAPTIASQPLSLRPEACRPAAGGAVHTRGLDFACDIYIWAPLAGTC |
| | | GVLLLSLVITLYCKRGRKKLLYIFKQPFMRPVQTTQEEDGCSCRFP |
| | | EEEEGGCELRVKFSRSADAPAYKQGQNQLYNELNLGRREEYDVL |
| | | DKRRGRDPEMGGKPRRKNPQEGLYNELQKDKMAEAYSEIGMKG |
| | | ERRRGKGHDGLYQGLSTATKDTYDALHMQALPPR |
| BCMA_EBB- | 248 | ATGGCCCTCCCTGTCACCGCCCTGCTGCTTCCGCTGGCTCTTCTGCTCC |
| C1980-G4-nt | | ACGCCGCTCGGCCCGAGGTGCAGTTGGTCGAAAGCGGGGGCGGGCTTGT |
| Full CART | | GCAGCCTGGCGGATCACTGCGGCTGTCCTGCGCGGCATCAGGCTTCACG |
| | | TTTTCTTCCTACGCCATGTCCTGGGTGCGCCAGGCCCCTGGAAAGGGAC |
| | | TGGAATGGGTGTCCGCGATTTCGGGGTCCGGCGGGAGCACCTACTACGC |
| | | CGATTCCGTGAAGGGCCGCTTCACTATCTCGCGGGACAACTCCAAGAAC |
| | | ACCCTCTACCTCCAAATGAATAGCCTGCGGGCCGAGGATACCGCCGTCT |
| | | ACTATTGCGCTAAGGTCGTGCGCGACGGAATGGACGTGTGGGGACAGGG |
| | | TACCACCGTGACAGTGTCCTCGGGGGGAGGCGGTAGCGGCGGAGGAGGA |
| | | AGCGGTGGTGGAGGTTCCGAGATTGTGCTGACTCAATCACCCGCGACCC |
| | | TGAGCCTGTCCCCCGGCGAAAGGGCCACTCTGTCCTGTCGGGCCAGCCA |
| | | ATCAGTCTCCTCCTCGTACCTGGCCTGGTACCAGCAGAAGCCAGGACAG |
| | | GCTCCGAGACTCCTTATCTATGGCGCATCCTCCCGCGCCACCGGAATCC |
| | | CGGATAGGTTCTCGGGAAACGGATCGGGGACCGACTTCACTCTCACCAT |
| | | CTCCCGGCTGGAACCGGAGGACTTCGCCGTGTACTACTGCCAGCAGTAC |
| | | GGCAGCCCGCCTAGATTCACTTTCGGCCCCGGCACCAAAGTGGACATCA |
| | | AGACCACTACCCCAGCACCGAGGCCACCCACCCCGGCTCCTACCATCGC |
| | | CTCCCAGCCTCTGTCCCTGCGTCCGGAGGCATGTAGACCCGCAGCTGGT |
| | | GGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTT |
| | | CATGCCGGTTCCCAGAGGAGGAGGAAGGCGGCTGCGAACTGCGCGTGAA |
| | | ATTCAGCCGCAGCGCAGATGCTCCAGCCTACAAGCAGGGGCAGAACCAG |
| | | CTCTACAACGAACTCAATCTTGGTCGGAGAGAGGAGTACGACGTGCTGG |
| | | ACAAGCGGAGAGGACGGGACCCAGAAATGGGCGGGAAGCCGCGCAGAAA |
| | | GAATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAAG |
| | | GCCACGACGGACTGTACCAGGGACTCAGCACCGCCACCAAGGACACCTA |
| | | TGACGCTCTTCACATGCAGGCCCTGCCGCCTCGG |

BCMA_EBB-C1980-D2

| | | |
|---|---|---|
| BCMA_EBB- | 144 | EVQLLESGGGLVQPGGSLRLSCAASGFTFSSYAMSWVRQAPGKGLEWVS |
| C1980-D2-aa | | AISGSGGSTYYADSVKGRFTISRDNSKNTLYLQMNSLRAEDTAVYYCAK |
| ScFv domain | | IPQTGTFDYWGQGTLVTVSSGGGGSGGGGSGGGGSEIVLTQSPGTLSLS |
| | | PGERATLSCRASQSVSSSYLAWYQQRPGQAPRLLIYGASSRATGIPDRF |
| | | SGSGSGTDFTLTISRLEPEDFAVYYCQHYGSSPSWTFGQGTRLEIK |
| BCMA_EBB- | 165 | GAAGTGCAGCTGCTGGAGTCCGGCGGTGGATTGGTGCAACCGGGGGGAT |
| C1980-D2-nt | | CGCTCAGACTGTCCTGTGCGGCGTCAGGCTTCACCTTCTCGAGCTACGC |
| ScFv domain | | CATGTCATGGGTCAGACAGGCCCCTGGAAAGGGTCTGGAATGGGTGTCC |
| | | GCCATTTCCGGGAGCGGGGGATCTACATACTACGCCGATAGCGTGAAGG |
| | | GCCGCTTCACCATTTCCCGGGACAACTCCAAGAACACTCTCTATCTGCA |
| | | AATGAACTCCCTCCGCGCTGAGGACACTGCCGTGTACTACTGCGCCAAA |
| | | ATCCCTCAGACCGGCACCTTCGACTACTGGGGACAGGGGACTCTGGTCA |
| | | CCGTCAGCAGCGGTGGCGGAGGTTCGGGGGGAGGAGGAAGCGGCGGCGG |
| | | AGGGTCCGAGATTGTGCTGACCCAGTCACCCGGCACTTTGTCCCTGTCG |
| | | CCTGGAGAAAGGGCCACCCTTTCCTGCCGGGCATCCCAATCCGTGTCCT |
| | | CCTCGTACCTGGCCTGGTACCAGCAGAGGCCCGGACAGGCCCCACGGCT |
| | | TCTGATCTACGGAGCAAGCAGCCGCGCGACCGGTATCCCGGACCGGTTT |
| | | TCGGGCTCGGGCTCAGGAACTGACTTCACCCTCACCATCTCCCGCCTGG |
| | | AACCCGAAGATTTCGCTGTGTATTACTGCCAGCACTACGGCAGCTCCCC |
| | | GTCCTGGACGTTCGGCCAGGGAACTCGGCTGGAGATCAAG |
| BCMA_EBB- | 186 | EVQLLESGGGLVQPGGSLRLSCAASGFTFSSYAMSWVRQAPGKGLEWVS |
| C1980-D2-aa | | AISGSGGSTYYADSVKGRFTISRDNSKNTLYLQMNSLRAEDTAVYYCAK |
| VH | | IPQTGTFDYWGQGTLVTVSS |
| BCMA_EBB- | 207 | EIVLTQSPGTLSLSPGERATLSCRASQSVSSSYLAWYQQRPGQAPRLLI |
| C1980-D2-aa | | YGASSRATGIPDRFSGSGSGTDFTLTISRLEPEDFAVYYCQHYGSSPSW |
| VL | | TFGQGTRLEIK |
| BCMA_EBB- | 228 | MALPVTALLLPLALLLHAARPEVQLLESGGGLVQPGGSLRLSCAASGFT |
| C1980-D2-aa | | FSSYAMSWVRQAPGKGLEWVSAISGSGGSTYYADSVKGRFTISRDNSKN |
| Full CART | | TLYLQMNSLRAEDTAVYYCAKIPQTGTFDYWGQGTLVTVSSGGGGSGGG |
| | | GSGGGGSEIVLTQSPGTLSLSPGERATLSCRASQSVSSSYLAWYQQRPG |
| | | QAPRLLIYGASSRATGIPDRFSGSGSGTDFTLTISRLEPEDFAVYYCQH |
| | | YGSSPSWTFGQGTRLEIKTTTPAPRPPTPAPTIASQPLSLRPEACRPAA |
| | | GGAVHTRGLDFACDIYIWAPLAGTCGVLLLSLVITLYCKRGRKKLLYIF |
| | | KQPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| BCMA_EBB- | 249 | ATGGCCCTCCCTGTCACCGCCCTGCTGCTTCCGCTGGCTCTTCTGCTCC |
| C1980-D2-nt | | ACGCCGCTCGGCCCGAAGTGCAGCTGCTGGAGTCCGGCGGTGGATTGGT |
| Full CART | | GCAACCGGGGGGATCGCTCAGACTGTCCTGTGCGGCGTCAGGCTTCACC |
| | | TTCTCGAGCTACGCCATGTCATGGGTCAGACAGGCCCCTGGAAAGGGTC |
| | | TGGAATGGGTGTCCGCCATTTCCGGGAGCGGGGGATCTACATACTACGC |
| | | CGATAGCGTGAAGGGCCGCTTCACCATTTCCCGGGACAACTCCAAGAAC |
| | | ACTCTCTATCTGCAAATGAACTCCCTCCGCGCTGAGGACACTGCCGTGT |
| | | ACTACTGCGCCAAAATCCCTCAGACCGGCACCTTCGACTACTGGGGACA |
| | | GGGGACTCTGGTCACCGTCAGCAGCGGTGGCGGAGGTTCGGGGGGAGGA |
| | | GGAAGCGGCGGCGGAGGGTCCGAGATTGTGCTGACCCAGTCACCCGGCA |
| | | CTTTGTCCCTGTCGCCTGGAGAAAGGGCCACCCTTTCCTGCCGGGCATC |
| | | CCAATCCGTGTCCTCCTCGTACCTGGCCTGGTACCAGCAGAGGCCCGGA |
| | | CAGGCCCCACGGCTTCTGATCTACGGAGCAAGCAGCCGCGCGACCGGTA |
| | | TCCCGGACCGGTTTTCGGGCTCGGGCTCAGGAACTGACTTCACCCTCAC |
| | | CATCTCCCGCCTGGAACCCGAAGATTTCGCTGTGTATTACTGCCAGCAC |
| | | TACGGCAGCTCCCCGTCCTGGACGTTCGGCCAGGGAACTCGGCTGGAGA |
| | | TCAAGACCACTACCCCAGCACCGAGGCCACCCACCCCGGCTCCTACCAT |
| | | CGCCTCCCAGCCTCTGTCCCTGCGTCCGGAGGCATGTAGACCCGCAGCT |
| | | GGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGT |
| | | GATCACTCTTTACTGTAAGCGCGGTCGGAAGAAGCTGCTGTACATCTTT |
| | | AAGCAACCCTTCATGAGGCCTGTGCAGACTACTCAAGAGGAGGACGGCT |
| | | GTTCATGCCGGTTCCCAGAGGAGGAGGAAGGCGGCTGCGAACTGCGCGT |
| | | GAAATTCAGCCGCAGCGCAGATGCTCCAGCCTACAAGCAGGGGCAGAAC |
| | | CAGCTCTACAACGAACTCAATCTTGGTCGGAGAGAGGAGTACGACGTGC |
| | | TGGACAAGCGGAGAGGACGGGACCCAGAAATGGGCGGGAAGCCGCGCAG |
| | | AAAGAATCCCCAAGAGGGCCTGTACAACGAGCTCCAAAAGGATAAGATG |
| | | GCAGAAGCCTATAGCGAGATTGGTATGAAAGGGGAACGCAGAAGAGGCA |
| | | AAGGCCACGACGGACTGTACCAGGGACTCAGCACCGCCACCAAGGACAC |
| | | CTATGACGCTCTTCACATGCAGGCCCTGCCGCCTCGG |

BCMA_EBB-C1978-A10

| | | |
|---|---|---|
| BCMA_EBB- | 145 | EVQLVETGGGLVQPGGSLRLSCAASGFTFSSYAMSWVRQAPGKG |
| C1978-A10- | | LEWVSAISGSGGSTYYADSVKGRFTMSRENDKNSVFLQMNSLRVE |
| aa | | DTGVYYCARANYKRELRYYYGMDVWGQGTMVTVSSGGGGSGG |
| ScFv domain | | GGSGGGGSEIVMTQSPGTLSLSPGESATLSCRASQRVASNYLAWY |
| | | QHKPGQAPSLLISGASSRATGVPDRFSGSGSGTDFTLAISRLEPEDS |
| | | AVYYCQHYDSSPSWTFGQGTKVEIK |
| BCMA_EBB- | 166 | GAAGTGCAACTGGTGGAAACCGGTGGAGGACTCGTGCAGCCTGGCGGCA |
| C1978-A10- | | GCCTCCGGCTGAGCTGCGCCGCTTCGGGATTCACCTTTTCCTCCTACGC |
| nt | | GATGTCTTGGGTCAGACAGGCCCCCGGAAAGGGGCTGGAATGGGTGTCA |
| ScFv domain | | GCCATCTCCGGCTCCGGCGGATCAACGTACTACGCCGACTCCGTGAAAG |
| | | GCCGGTTCACCATGTCGCGCGAGAATGACAAGAACTCCGTGTTCCTGCA |
| | | AATGAACTCCCTGAGGGTGGAGGACACCGGAGTGTACTATTGTGCGCGC |
| | | GCCAACTACAAGAGAGAGCTGCGGTACTACTACGGAATGGACGTCTGGG |
| | | GACAGGGAACTATGGTGACCGTGTCATCCGGTGGAGGGGGAAGCGGCGG |
| | | TGGAGGCAGCGGGGGCGGGGGTTCAGAAATTGTCATGACCCAGTCCCCG |
| | | GGAACTCTTTCCCTCTCCCCCGGGGAATCCGCGACTTTGTCCTGCCGGG |
| | | CCAGCCAGCGCGTGGCCTCGAACTACCTCGCATGGTACCAGCATAAGCC |
| | | AGGCCAAGCCCCTTCCCTGCTGATTTCCGGGGCTAGCAGCCGCGCCACT |
| | | GGCGTGCCGGATAGGTTCTCGGGAAGCGGCTCGGGTACCGATTTCACCC |
| | | TGGCAATCTCGCGGCTGGAACCGGAGGATTCGGCCGTGTACTACTGCCA |
| | | GCACTATGACTCATCCCCCTCCTGGACATTCGGACAGGGCACCAAGGTC |
| | | GAGATCAAG |
| BCMA_EBB- | 187 | EVQLVETGGGLVQPGGSLRLSCAASGFTFSSYAMSWVRQAPGKGLEWVS |
| C1978-A10- | | AISGSGGSTYYADSVKGRFTMSRENDKNSVFLQMNSLRVEDTGVYYCAR |
| aa | | ANYKRELRYYYGMDVWGQGTMVTVSS |
| VH | | |
| BCMA_EBB- | 208 | EIVMTQSPGTLSLSPGESATLSCRASQRVASNYLAWYQHKPGQAPSLLI |
| C1978-A10- | | SGASSRATGVPDRFSGSGSGTDFTLAISRLEPEDSAVYYCQHYDSSPSW |
| aa | | TFGQGTKVEIK |
| VL | | |
| BCMA_EBB- | 229 | MALPVTALLLPLALLLHAARPEVQLVETGGGLVQPGGSLRLSCAA |
| C1978-A10- | | SGFTFSSYAMSWVRQAPGKGLEWVSAISGSGGSTYYADSVKGRFT |
| aa | | MSRENDKNSVFLQMNSLRVEDTGVYYCARANYKRELRYYYGMD |
| Full CART | | VWGQGTMVTVSSGGGGSGGGGSGGGGSEIVMTQSPGTLSLSPGES |
| | | ATLSCRASQRVASNYLAWYQHKPGQAPSLLISGASSRATGVPDRF |
| | | SGSGSGTDFTLAISRLEPEDSAVYYCQHYDSSPSWTFGQGTKVEIK |
| | | TTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLG |
| | | RREEYDVLDKRRGRDPEMGGKPRRKNPQEGLYNELQKDKMAEA |
| | | YSEIGMKGERRRGKGHDGLYQGLSTATKDTYDALHMQALPPR |
| BCMA_EBB- | 250 | ATGGCCCTCCCTGTCACCGCCCTGCTGCTTCCGCTGGCTCTTCTGCTCC |
| C1978-A10- | | ACGCCGCTCGGCCCGAAGTGCAACTGGTGGAAACCGGTGGAGGACTCGT |
| nt | | GCAGCCTGGCGGCAGCCTCCGGCTGAGCTGCGCCGCTTCGGGATTCACC |
| Full CART | | TTTTCCTCCTACGCGATGTCTTGGGTCAGACAGGCCCCCGGAAAGGGGC |
| | | TGGAATGGGTGTCAGCCATCTCCGGCTCCGGCGGATCAACGTACTACGC |
| | | CGACTCCGTGAAAGGCCGGTTCACCATGTCGCGCGAGAATGACAAGAAC |
| | | TCCGTGTTCCTGCAAATGAACTCCCTGAGGGTGGAGGACACCGGAGTGT |
| | | ACTATTGTGCGCGCGCCAACTACAAGAGAGAGCTGCGGTACTACTACGG |
| | | AATGGACGTCTGGGGACAGGGAACTATGGTGACCGTGTCATCCGGTGGA |
| | | GGGGGAAGCGGCGGTGGAGGCAGCGGGGGCGGGGGTTCAGAAATTGTCA |
| | | TGACCCAGTCCCCGGGAACTCTTTCCCTCTCCCCCGGGGAATCCGCGAC |
| | | TTTGTCCTGCCGGGCCAGCCAGCGCGTGGCCTCGAACTACCTCGCATGG |
| | | TACCAGCATAAGCCAGGCCAAGCCCCTTCCCTGCTGATTTCCGGGGCTA |
| | | GCAGCCGCGCCACTGGCGTGCCGGATAGGTTCTCGGGAAGCGGCTCGGG |
| | | TACCGATTTCACCCTGGCAATCTCGCGGCTGGAACCGGAGGATTCGGCC |
| | | GTGTACTACTGCCAGCACTATGACTCATCCCCCTCCTGGACATTCGGAC |
| | | AGGGCACCAAGGTCGAGATCAAGACCACTACCCCAGCACCGAGGCCACC |
| | | CACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACT |
| | | TCGCCTGCGATATCTACATTTGGGCCCCTCTGGCTGGTACTTGCGGGGT |
| | | CCTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACTA |
| | | CTCAAGAGGAGGACGGCTGTTCATGCCGGTTCCCAGAGGAGGAGGAAGG |
| | | CGGCTGCGAACTGCGCGTGAAATTCAGCCGCAGCGCAGATGCTCCAGCC |
| | | TACAAGCAGGGGCAGAACCAGCTCTACAACGAACTCAATCTTGGTCGGA |
| | | GAGAGGAGTACGACGTGCTGGACAAGCGGAGAGGACGGGACCCAGAAAT |
| | | GGGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAAG |
| | | GGGAACGCAGAAGAGGCAAAGGCCACGACGGACTGTACCAGGGACTCAG |
| | | CACCGCCACCAAGGACACCTATGACGCTCTTCACATGCAGGCCCTGCCG |
| | | CCTCGG |

BCMA_EBB-C1978-D4

| | | |
|---|---|---|
| BCMA_EBB- | 146 | EVQLLETGGGLVQPGGSLRLSCAASGFSFSSYAMSWVRQAPGKGLEWVS |
| C1978-D4-aa | | AISGSGGSTYYADSVKGRFTISRDNSKNTLYLQMNSLRAEDTAVYYCAK |
| ScFv domain | | ALVGATGAFDIWGQGTLVTVSSGGGGSGGGGSGGGGSEIVLTQSPGTLS |
| | | LSPGERATLSCRASQSLSSNFLAWYQQKPGQAPGLLIYGASNWATGTPD |
| | | RFSGSGSGTDFTLTITRLEPEDFAVYYCQYYGTSPMYTFGQGTKVEIK |
| BCMA_EBB- | 167 | GAAGTGCAGCTGCTCGAAACCGGTGGAGGGCTGGTGCAGCCAGGGGGCT |
| C1978-D4-nt | | CCCTGAGGCTTTCATGCGCCGCTAGCGGATTCTCCTTCTCCTCTTACGC |
| ScFv domain | | CATGTCGTGGGTCCGCCAAGCCCCTGGAAAAGGCCTGGAATGGGTGTCC |
| | | GCGATTTCCGGGAGCGGAGGTTCGACCTATTACGCCGACTCCGTGAAGG |
| | | GCCGCTTTACCATCTCCCGGGATAACTCCAAGAACACTCTGTACCTCCA |
| | | AATGAACTCGCTGAGAGCCGAGGACACCGCCGTGTATTACTGCGCGAAG |
| | | GCGCTGGTCGGCGCGACTGGGGCATTCGACATCTGGGGACAGGGAACTC |
| | | TTGTGACCGTGTCGAGCGGAGGCGGCGGCTCCGGCGGAGGAGGGAGCGG |
| | | GGGCGGTGGTTCCGAAATCGTGTTGACTCAGTCCCCGGGAACCCTGAGC |
| | | TTGTCACCCGGGGAGCGGGCCACTCTCTCCTGTCGCGCCTCCCAATCGC |
| | | TCTCATCCAATTTCCTGGCCTGGTACCAGCAGAAGCCCGGACAGGCCCC |
| | | GGGCCTGCTCATCTACGGCGCTTCAAACTGGGCAACGGGAACCCCTGAT |
| | | CGGTTCAGCGGAAGCGGATCGGGTACTGACTTTACCCTGACCATCACCA |
| | | GACTGGAACCGGAGGACTTCGCCGTGTACTACTGCCAGTACTACGGCAC |
| | | CTCCCCCATGTACACATTCGGACAGGGTACCAAGGTCGAGATTAAG |
| BCMA_EBB- | 188 | EVQLLETGGGLVQPGGSLRLSCAASGFSFSSYAMSWVRQAPGKGLEWVS |
| C1978-D4-aa | | AISGSGGSTYYADSVKGRFTISRDNSKNTLYLQMNSLRAEDTAVYYCAK |
| VH | | ALVGATGAFDIWGQGTLVTVSS |
| BCMA_EBB- | 209 | EIVLTQSPGTLSLSPGERATLSCRASQSLSSNFLAWYQQKPGQAPGLLI |
| C1978-D4-aa | | YGASNWATGTPDRFSGSGSGTDFTLTITRLEPEDFAVYYCQYYGTSPMY |
| VL | | TFGQGTKVEIK |
| BCMA_EBB- | 230 | MALPVTALLLPLALLLHAARPEVQLLETGGGLVQPGGSLRLSCAASGFS |
| C1978-D4-aa | | FSSYAMSWVRQAPGKGLEWVSAISGSGGSTYYADSVKGRFTISRDNSKN |
| Full CART | | TLYLQMNSLRAEDTAVYYCAKALVGATGAFDIWGQGTLVTVSSGGGGSG |
| | | GGGSGGGGSEIVLTQSPGTLSLSPGERATLSCRASQSLSSNFLAWYQQK |
| | | PGQAPGLLIYGASNWATGTPDRFSGSGSGTDFTLTITRLEPEDFAVYYC |
| | | QYYGTSPMYTFGQGTKVEIKTTTPAPRPPTPAPTIASQPLSLRPEACRP |
| | | AAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| BCMA_EBB- | 251 | ATGGCCCTCCCTGTCACCGCCCTGCTGCTTCCGCTGGCTCTTCTGCTCC |
| C1978-D4-nt | | ACGCCGCTCGGCCCGAAGTGCAGCTGCTCGAAACCGGTGGAGGGCTGGT |
| Full CART | | GCAGCCAGGGGGCTCCCTGAGGCTTTCATGCGCCGCTAGCGGATTCTCC |
| | | TTCTCCTCTTACGCCATGTCGTGGGTCCGCCAAGCCCCTGGAAAAGGCC |
| | | TGGAATGGGTGTCCGCGATTTCCGGGAGCGGAGGTTCGACCTATTACGC |
| | | CGACTCCGTGAAGGGCCGCTTTACCATCTCCCGGGATAACTCCAAGAAC |
| | | ACTCTGTACCTCCAAATGAACTCGCTGAGAGCCGAGGACACCGCCGTGT |
| | | ATTACTGCGCGAAGGCGCTGGTCGGCGCGACTGGGGCATTCGACATCTG |
| | | GGGACAGGGAACTCTTGTGACCGTGTCGAGCGGAGGCGGCGGCTCCGGC |
| | | GGAGGAGGGAGCGGGGGCGGTGGTTCCGAAATCGTGTTGACTCAGTCCC |
| | | CGGGAACCCTGAGCTTGTCACCCGGGGAGCGGGCCACTCTCTCCTGTCG |
| | | CGCCTCCCAATCGCTCTCATCCAATTTCCTGGCCTGGTACCAGCAGAAG |
| | | CCCGGACAGGCCCCGGGCCTGCTCATCTACGGCGCTTCAAACTGGGCAA |
| | | CGGGAACCCCTGATCGGTTCAGCGGAAGCGGATCGGGTACTGACTTTAC |
| | | CCTGACCATCACCAGACTGGAACCGGAGGACTTCGCCGTGTACTACTGC |
| | | CAGTACTACGGCACCTCCCCCATGTACACATTCGGACAGGGTACCAAGG |
| | | TCGAGATTAAGACCACTACCCCAGCACCGAGGCCACCCACCCCGGCTCC |
| | | TACCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCGATA |
| | | TCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTAC |
| | | ATCTTTAAGCAACCCTTCATGAGGCCTGTGCAGACTACTCAAGAGGAGG |
| | | ACGGCTGTTCATGCCGGTTCCCAGAGGAGGAGGAAGGCGGCTGCGAACT |
| | | GCGCGTGAAATTCAGCCGCAGCGCAGATGCTCCAGCCTACAAGCAGGGG |
| | | CAGAACCAGCTCTACAACGAACTCAATCTTGGTCGGAGAGAGGAGTACG |
| | | ACGTGCTGGACAAGCGGAGAGGACGGGACCCAGAAATGGGCGGGAAGCC |
| | | GCGCAGAAAGAATCCCCAAGAGGGCCTGTACAACGAGCTCCAAAAGGAT |
| | | AAGATGGCAGAAGCCTATAGCGAGATTGGTATGAAAGGGGAACGCAGAA |
| | | GAGGCAAAGGCCACGACGGACTGTACCAGGGACTCAGCACCGCCACCAA |
| | | GGACACCTATGACGCTCTTCACATGCAGGCCCTGCCGCCTCGG |

BCMA_EBB-C1980-A2

| | | |
|---|---|---|
| BCMA_EBB- | 147 | EVQLLESGGGLVQPGGSLRLSCAASGFTFSSYAMSWVRQAPGKGLEWVS |
| C1980-A2-aa | | AISGSGGSTYYADSVKGRFTISRDNSKNTLYLQMNSLRAEDTAVYYCVL |
| ScFv domain | | WFGEGFDPWGQGTLVTVSSGGGGSGGGGSGGGGSDIVLTQSPLSLPVTP |
| | | GEPASISCRSSQSLLHSNGYNYLDWYLQKPGQSPQLLIYLGSNRASGVP |
| | | DRFSGSGSGTDFTLKISRVEAEDVGVYYCMQALQTPLTFGGGTKVDIK |
| BCMA_EBB- | 168 | GAAGTGCAGCTGCTTGAGAGCGGTGGAGGTCTGGTGCAGCCCGGGGGAT |
| C1980-A2-nt | | CACTGCGCCTGTCCTGTGCCGCGTCCGGTTTCACTTTCTCCTCGTACGC |
| ScFv domain | | CATGTCGTGGGTCAGACAGGCACCGGGAAAGGGACTGGAATGGGTGTCA |
| | | GCCATTTCGGGTTCGGGGGGCAGCACCTACTACGCTGACTCCGTGAAGG |
| | | GCCGGTTCACCATTTCCCGCGACAACTCCAAGAACACCTTGTACCTCCA |
| | | AATGAACTCCCTGCGGGCCGAAGATACCGCCGTGTATTACTGCGTGCTG |
| | | TGGTTCGGAGAGGGATTCGACCCGTGGGGACAAGGAACACTCGTGACTG |
| | | TGTCATCCGGCGGAGGCGGCAGCGGTGGCGGCGGTTCCGGCGGCGGCGG |
| | | ATCTGACATCGTGTTGACCCAGTCCCCTCTGAGCCTGCCGGTCACTCCT |
| | | GGCGAACCAGCCAGCATCTCCTGCCGGTCGAGCCAGTCCCTCCTGCACT |
| | | CCAATGGGTACAACTACCTCGATTGGTATCTGCAAAAGCCGGGCCAGAG |
| | | CCCCCAGCTGCTGATCTACCTTGGGTCAAACCGCGCTTCCGGGGTGCCT |
| | | GATAGATTCTCCGGGTCCGGGAGCGGAACCGACTTTACCCTGAAAATCT |
| | | CGAGGGTGGAGGCCGAGGACGTCGGAGTGTACTACTGCATGCAGGCGCT |
| | | CCAGACTCCCCTGACCTTCGGAGGAGGAACGAAGGTCGACATCAAGA |
| BCMA_EBB- | 189 | EVQLLESGGGLVQPGGSLRLSCAASGFTFSSYAMSWVRQAPGKGLEWVS |
| C1980-A2-aa | | AISGSGGSTYYADSVKGRFTISRDNSKNTLYLQMNSLRAEDTAVYYCVL |
| VH | | WFGEGFDPWGQGTLVTVSS |
| BCMA_EBB- | 210 | DIVLTQSPLSLPVTPGEPASISCRSSQSLLHSNGYNYLDWYLQKPGQSP |
| C1980-A2-aa | | QLLIYLGSNRASGVPDRFSGSGSGTDFTLKISRVEAEDVGVYYCMQALQ |
| VL | | TPLTFGGGTKVDIK |
| BCMA_EBB- | 231 | MALPVTALLLPLALLLHAARPEVQLLESGGGLVQPGGSLRLSCAASGFT |
| C1980-A2-aa | | FSSYAMSWVRQAPGKGLEWVSAISGSGGSTYYADSVKGRFTISRDNSKN |
| Full CART | | TLYLQMNSLRAEDTAVYYCVLWFGEGFDPWGQGTLVTVSSGGGGSGGGG |
| | | SGGGGSDIVLTQSPLSLPVTPGEPASISCRSSQSLLHSNGYNYLDWYLQ |
| | | KPGQSPQLLIYLGSNRASGVPDRFSGSGSGTDFTLKISRVEAEDVGVYY |
| | | CMQALQTPLTFGGGTKVDIKTTTPAPRPPTPAPTIASQPLSLRPEACRP |
| | | AAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| BCMA_EBB- | 252 | ATGGCCCTCCCTGTCACCGCCCTGCTGCTTCCGCTGGCTCTTCTGCTCC |
| C1980-A2-nt | | ACGCCGCTCGGCCCGAAGTGCAGCTGCTTGAGAGCGGTGGAGGTCTGGT |
| Full CART | | GCAGCCCGGGGGATCACTGCGCCTGTCCTGTGCCGCGTCCGGTTTCACT |
| | | TTCTCCTCGTACGCCATGTCGTGGGTCAGACAGGCACCGGGAAAGGGAC |
| | | TGGAATGGGTGTCAGCCATTTCGGGTTCGGGGGGCAGCACCTACTACGC |
| | | TGACTCCGTGAAGGGCCGGTTCACCATTTCCCGCGACAACTCCAAGAAC |
| | | ACCTTGTACCTCCAAATGAACTCCCTGCGGGCCGAAGATACCGCCGTGT |
| | | ATTACTGCGTGCTGTGGTTCGGAGAGGGATTCGACCCGTGGGGACAAGG |
| | | AACACTCGTGACTGTGTCATCCGGCGGAGGCGGCAGCGGTGGCGGCGGT |
| | | TCCGGCGGCGGCGGATCTGACATCGTGTTGACCCAGTCCCCTCTGAGCC |
| | | TGCCGGTCACTCCTGGCGAACCAGCCAGCATCTCCTGCCGGTCGAGCCA |
| | | GTCCCTCCTGCACTCCAATGGGTACAACTACCTCGATTGGTATCTGCAA |
| | | AAGCCGGGCCAGAGCCCCCAGCTGCTGATCTACCTTGGGTCAAACCGCG |
| | | CTTCCGGGGTGCCTGATAGATTCTCCGGGTCCGGGAGCGGAACCGACTT |
| | | TACCCTGAAAATCTCGAGGGTGGAGGCCGAGGACGTCGGAGTGTACTAC |
| | | TGCATGCAGGCGCTCCAGACTCCCCTGACCTTCGGAGGAGGAACGAAGG |
| | | TCGACATCAAGACCACTACCCCAGCACCGAGGCCACCCACCCCGGCTCC |
| | | TACCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCGATA |
| | | TCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTAC |
| | | ATCTTTAAGCAACCCTTCATGAGGCCTGTGCAGACTACTCAAGAGGAGG |
| | | ACGGCTGTTCATGCCGGTTCCCAGAGGAGGAGGAAGGCGGCTGCGAACT |
| | | GCGCGTGAAATTCAGCCGCAGCGCAGATGCTCCAGCCTACAAGCAGGGG |
| | | CAGAACCAGCTCTACAACGAACTCAATCTTGGTCGGAGAGAGGAGTACG |
| | | ACGTGCTGGACAAGCGGAGAGGACGGGACCCAGAAATGGGCGGGAAGCC |
| | | GCGCAGAAAGAATCCCCAAGAGGGCCTGTACAACGAGCTCCAAAAGGAT |
| | | AAGATGGCAGAAGCCTATAGCGAGATTGGTATGAAAGGGGAACGCAGAA |
| | | GAGGCAAAGGCCACGACGGACTGTACCAGGGACTCAGCACCGCCACCAA |
| | | GGACACCTATGACGCTCTTCACATGCAGGCCCTGCCGCCTCGG |

BCMA_EBB-C1981-C3

| | | |
|---|---|---|
| BCMA_EBB- | 148 | QVQLVESGGGLVQPGGSLRLSCAASGFTFSSYAMSWVRQAPGKGLEWVS |
| C1981-C3-aa | | AISGSGGSTYYADSVKGRFTISRDNSKNTLYLQMNSLRAEDTAVYYCAK |
| ScFv domain | | VGYDSSGYYRDYYGMDVWGQGTTVTVSSGGGGSGGGGSGGGGSEIVLTQ |
| | | SPGTLSLSPGERATLSCRASQSVSSSYLAWYQQKPGQAPRLLIYGTSSR |
| | | ATGISDRFSGSGSGTDFTLTISRLEPEDFAVYYCQHYGNSPPKFTFGPG |
| | | TKLEIK |
| BCMA_EBB- | 169 | CAAGTGCAGCTCGTGGAGTCAGGCGGAGGACTGGTGCAGCCCGGGGGCT |
| C1981-C3-nt | | CCCTGAGACTTTCCTGCGCGGCATCGGGTTTTACCTTCTCCTCCTATGC |
| ScFv domain | | TATGTCCTGGGTGCGCCAGGCCCCGGGAAAGGGACTGGAATGGGTGTCC |
| | | GCAATCAGCGGTAGCGGGGGCTCAACATACTACGCCGACTCCGTCAAGG |
| | | GTCGCTTCACTATTTCCCGGGACAACTCCAAGAATACCCTGTACCTCCA |
| | | AATGAACAGCCTCAGGGCCGAGGATACTGCCGTGTACTACTGCGCCAAA |
| | | GTCGGATACGATAGCTCCGGTTACTACCGGGACTACTACGGAATGGACG |
| | | TGTGGGGACAGGGCACCACCGTGACCGTGTCAAGCGGCGGAGGCGGTTC |
| | | AGGAGGGGGAGGCTCCGGCGGTGGAGGGTCCGAAATCGTCCTGACTCAG |
| | | TCGCCTGGCACTCTGTCGTTGTCCCCGGGGGAGCGCGCTACCCTGTCGT |
| | | GTCGGGCGTCGCAGTCCGTGTCGAGCTCCTACCTCGCGTGGTACCAGCA |
| | | GAAGCCCGGACAGGCCCCTAGACTTCTGATCTACGGCACTTCTTCACGC |
| | | GCCACCGGGATCAGCGACAGGTTCAGCGGCTCCGGCTCCGGGACCGACT |
| | | TCACCCTGACCATTAGCCGGCTGGAGCCTGAAGATTTCGCCGTGTATTA |
| | | CTGCCAACACTACGGAAACTCGCCGCCAAAGTTCACGTTCGGACCCGGA |
| | | ACCAAGCTGGAAATCAAG |
| BCMA_EBB- | 190 | QVQLVESGGGLVQPGGSLRLSCAASGFTFSSYAMSWVRQAPGKGLEWVS |
| C1981-C3-aa | | AISGSGGSTYYADSVKGRFTISRDNSKNTLYLQMNSLRAEDTAVYYCAK |
| VH | | VGYDSSGYYRDYYGMDVWGQGTTVTVSS |
| BCMA_EBB- | 211 | EIVLTQSPGTLSLSPGERATLSCRASQSVSSSYLAWYQQKPGQAPRLLI |
| C1981-C3-aa | | YGTSSRATGISDRFSGSGSGTDFTLTISRLEPEDFAVYYCQHYGNSPPK |
| VL | | FTFGPGTKLEIK |
| BCMA_EBB- | 232 | MALPVTALLLPLALLLHAARPQVQLVESGGGLVQPGGSLRLSCAASGFT |
| C1981-C3-aa | | FSSYAMSWVRQAPGKGLEWVSAISGSGGSTYYADSVKGRFTISRDNSKN |
| Full CART | | TLYLQMNSLRAEDTAVYYCAKVGYDSSGYYRDYYGMDVWGQGTTVTVSS |
| | | GGGGSGGGGSGGGGSEIVLTQSPGTLSLSPGERATLSCRASQSVSSSYL |
| | | AWYQQKPGQAPRLLIYGTSSRATGISDRFSGSGSGTDFTLTISRLEPED |
| | | FAVYYCQHYGNSPPKFTFGPGTKLEIKTTTPAPRPPTPAPTIASQPLSL |
| | | RPEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSAD |
| | | APAYKQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHMQ |
| | | ALPPR |
| BCMA_EBB- | 253 | ATGGCCCTCCCTGTCACCGCCCTGCTGCTTCCGCTGGCTCTTCTGCTCC |
| C1981-C3-nt | | ACGCCGCTCGGCCCCAAGTGCAGCTCGTGGAGTCAGGCGGAGGACTGGT |
| Full CART | | GCAGCCCGGGGGCTCCCTGAGACTTTCCTGCGCGGCATCGGGTTTTACC |
| | | TTCTCCTCCTATGCTATGTCCTGGGTGCGCCAGGCCCCGGGAAAGGGAC |
| | | TGGAATGGGTGTCCGCAATCAGCGGTAGCGGGGGCTCAACATACTACGC |
| | | CGACTCCGTCAAGGGTCGCTTCACTATTTCCCGGGACAACTCCAAGAAT |
| | | ACCCTGTACCTCCAAATGAACAGCCTCAGGGCCGAGGATACTGCCGTGT |
| | | ACTACTGCGCCAAAGTCGGATACGATAGCTCCGGTTACTACCGGGACTA |
| | | CTACGGAATGGACGTGTGGGGACAGGGCACCACCGTGACCGTGTCAAGC |
| | | GGCGGAGGCGGTTCAGGAGGGGGAGGCTCCGGCGGTGGAGGGTCCGAAA |
| | | TCGTCCTGACTCAGTCGCCTGGCACTCTGTCGTTGTCCCCGGGGGAGCG |
| | | CGCTACCCTGTCGTGTCGGGCGTCGCAGTCCGTGTCGAGCTCCTACCTC |
| | | GCGTGGTACCAGCAGAAGCCCGGACAGGCCCCTAGACTTCTGATCTACG |
| | | GCACTTCTTCACGCGCCACCGGGATCAGCGACAGGTTCAGCGGCTCCGG |
| | | CTCCGGGACCGACTTCACCCTGACCATTAGCCGGCTGGAGCCTGAAGAT |
| | | TTCGCCGTGTATTACTGCCAACACTACGGAAACTCGCCGCCAAAGTTCA |
| | | CGTTCGGACCCGGAACCAAGCTGGAAATCAAGACCACTACCCCAGCACC |
| | | GAGGCCACCCACCCCGGCTCCTACCATCGCCTCCCAGCCTCTGTCCCTG |
| | | CGTCCGGAGGCATGTAGACCCGCAGCTGGTGGGGCCGTGCATACCCGGG |
| | | GTCTTGACTTCGCCTGCGATATCTACATTTGGGCCCCTCTGGCTGGTAC |
| | | TTGCGGGGTCCTGCTGCTTTCACTCGTGATCACTCTTTACTGTAAGCGC |
| | | GGTCGGAAGAAGCTGCTGTACATCTTTAAGCAACCCTTCATGAGGCCTG |
| | | TGCAGACTACTCAAGAGGAGGACGGCTGTTCATGCCGGTTCCCAGAGGA |
| | | GGAGGAAGGCGGCTGCGAACTGCGCGTGAAATTCAGCCGCAGCGCAGAT |
| | | GCTCCAGCCTACAAGCAGGGGCAGAACCAGCTCTACAACGAACTCAATC |
| | | TTGGTCGGAGAGAGGAGTACGACGTGCTGGACAAGCGGAGAGGACGGGA |
| | | CCCAGAAATGGGCGGGAAGCCGCGCAGAAAGAATCCCCAAGAGGGCCTG |
| | | TACAACGAGCTCCAAAAGGATAAGATGGCAGAAGCCTATAGCGAGATTG |
| | | GTATGAAAGGGGAACGCAGAAGAGGCAAAGGCCACGACGGACTGTACCA |
| | | GGGACTCAGCACCGCCACCAAGGACACCTATGACGCTCTTCACATGCAG |
| | | GCCCTGCCGCCTCGG |

BCMA_EBB-C1978-G4

| | | |
|---|---|---|
| BCMA_EBB- | 149 | EVQLVESGGGLVQPGGSLRLSCAASGFTFSSYAMSWVRQAPGKGLEWVS |
| C1978-G4-aa | | AISGSGGSTYYADSVKGRFTISRDNSKNTLYLQMNSLRAEDTAVYYCAK |
| ScFv domain | | MGWSSGYLGAFDIWGQGTTVTVSSGGGGSGGGGSGGGGSEIVLTQSPGT |
| | | LSLSPGERATLSCRASQSVASSFLAWYQQKPGQAPRLLIYGASGRATGI |
| | | PDRFSGSGSGTDFTLTISRLEPEDFAVYYCQHYGGSPRLTFGGGTKVDI |
| | | K |
| BCMA_EBB- | 170 | GAAGTCCAACTGGTGGAGTCCGGGGGAGGGCTCGTGCAGCCCGGAGGCA |
| C1978-G4-nt | | GCCTTCGGCTGTCGTGCGCCGCCTCCGGGTTCACGTTCTCATCCTACGC |
| ScFv domain | | GATGTCGTGGGTCAGACAGGCACCAGGAAAGGGACTGGAATGGGTGTCC |
| | | GCCATTAGCGGCTCCGGCGGTAGCACCTACTATGCCGACTCAGTGAAGG |
| | | GAAGGTTCACTATCTCCCGCGACAACAGCAAGAACACCCTGTACCTCCA |
| | | AATGAACTCTCTGCGGGCCGAGGATACCGCGGTGTACTATTGCGCCAAG |
| | | ATGGGTTGGTCCAGCGGATACTTGGGAGCCTTCGACATTTGGGGACAGG |
| | | GCACTACTGTGACCGTGTCCTCCGGGGGTGGCGGATCGGGAGGCGGCGG |
| | | CTCGGGTGGAGGGGGTTCCGAAATCGTGTTGACCCAGTCACCGGGAACC |
| | | CTCTCGCTGTCCCCGGGAGAACGGGCTACACTGTCATGTAGAGCGTCCC |
| | | AGTCCGTGGCTTCCTCGTTCCTGGCCTGGTACCAGCAGAAGCCGGGACA |
| | | GGCACCCCGCCTGCTCATCTACGGAGCCAGCGGCCGGGCGACCGGCATC |
| | | CCTGACCGCTTCTCCGGTTCCGGCTCGGGCACCGACTTTACTCTGACCA |
| | | TTAGCAGGCTTGAGCCCGAGGATTTTGCCGTGTACTACTGCCAACACTA |
| | | CGGGGGGAGCCCTCGCCTGACCTTCGGAGGCGGAACTAAGGTCGATATC |
| | | AAAA |
| BCMA_EBB- | 191 | EVQLVESGGGLVQPGGSLRLSCAASGFTFSSYAMSWVRQAPGKGLEWVS |
| C1978-G4-aa | | AISGSGGSTYYADSVKGRFTISRDNSKNTLYLQMNSLRAEDTAVYYCAK |
| VH | | MGWSSGYLGAFDIWGQGTTVTVSS |
| BCMA_EBB- | 212 | EIVLTQSPGTLSLSPGERATLSCRASQSVASSFLAWYQQKPGQAPRLLI |
| C1978-G4-aa | | YGASGRATGIPDRFSGSGSGTDFTLTISRLEPEDFAVYYCQHYGGSPRL |
| VL | | TFGGGTKVDIK |
| BCMA_EBB- | 233 | MALPVTALLLPLALLLHAARPEVQLVESGGGLVQPGGSLRLSCAASGFT |
| C1978-G4-aa | | FSSYAMSWVRQAPGKGLEWVSAISGSGGSTYYADSVKGRFTISRDNSKN |
| Full CART | | TLYLQMNSLRAEDTAVYYCAKMGWSSGYLGAFDIWGQGTTVTVSSGGGG |
| | | SGGGGSGGGGSEIVLTQSPGTLSLSPGERATLSCRASQSVASSFLAWYQ |
| | | QKPGQAPRLLIYGASGRATGIPDRFSGSGSGTDFTLTISRLEPEDFAVY |
| | | YCQHYGGSPRLTFGGGTKVDIKTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| BCMA_EBB- | 254 | ATGGCCCTCCCTGTCACCGCCCTGCTGCTTCCGCTGGCTCTTCTGCTCC |
| C1978-G4-nt | | ACGCCGCTCGGCCCGAAGTCCAACTGGTGGAGTCCGGGGGAGGGCTCGT |
| Full CART | | GCAGCCCGGAGGCAGCCTTCGGCTGTCGTGCGCCGCCTCCGGGTTCACG |
| | | TTCTCATCCTACGCGATGTCGTGGGTCAGACAGGCACCAGGAAAGGGAC |
| | | TGGAATGGGTGTCCGCCATTAGCGGCTCCGGCGGTAGCACCTACTATGC |
| | | CGACTCAGTGAAGGGAAGGTTCACTATCTCCCGCGACAACAGCAAGAAC |
| | | ACCCTGTACCTCCAAATGAACTCTCTGCGGGCCGAGGATACCGCGGTGT |
| | | ACTATTGCGCCAAGATGGGTTGGTCCAGCGGATACTTGGGAGCCTTCGA |
| | | CATTTGGGGACAGGGCACTACTGTGACCGTGTCCTCCGGGGGTGGCGGA |
| | | TCGGGAGGCGGCGGCTCGGGTGGAGGGGGTTCCGAAATCGTGTTGACCC |
| | | AGTCACCGGGAACCCTCTCGCTGTCCCCGGGAGAACGGGCTACACTGTC |
| | | ATGTAGAGCGTCCCAGTCCGTGGCTTCCTCGTTCCTGGCCTGGTACCAG |
| | | CAGAAGCCGGGACAGGCACCCCGCCTGCTCATCTACGGAGCCAGCGGCC |
| | | GGGCGACCGGCATCCCTGACCGCTTCTCCGGTTCCGGCTCGGGCACCGA |
| | | CTTTACTCTGACCATTAGCAGGCTTGAGCCCGAGGATTTTGCCGTGTAC |
| | | TACTGCCAACACTACGGGGGGAGCCCTCGCCTGACCTTCGGAGGCGGAA |
| | | CTAAGGTCGATATCAAAACCACTACCCCAGCACCGAGGCCACCCACCCC |
| | | GGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAG |
| | | AGGAGGACGGCTGTTCATGCCGGTTCCCAGAGGAGGAGGAAGGCGGCTG |
| | | CGAACTGCGCGTGAAATTCAGCCGCAGCGCAGATGCTCCAGCCTACAAG |
| | | CAGGGGCAGAACCAGCTCTACAACGAACTCAATCTTGGTCGGAGAGAGG |
| | | AGTACGACGTGCTGGACAAGCGGAGAGGACGGGACCCAGAAATGGGCGG |
| | | GAAGCCGCGCAGAAAGAATCCCCAAGAGGGCCTGTACAACGAGCTCCAA |
| | | AAGGATAAGATGGCAGAAGCCTATAGCGAGATTGGTATGAAAGGGGAAC |
| | | GCAGAAGAGGCAAAGGCCACGACGGACTGTACCAGGGACTCAGCACCGC |
| | | CACCAAGGACACCTATGACGCTCTTCACATGCAGGCCCTGCCGCCTCGG |

TABLE 3
Heavy Chain Variable Domain CDRs according to the Kabat numbering scheme
(Kabat et al. (1991), ″Sequences of Proteins of Immunological Interest,″
5th Ed. Public Health Service, National Institutes of Health, Bethesda, MD)

| | | | | SEQ | | SEQ |
|---|---|---|---|---|---|---|
| | | SEQ | | ID | | ID |
| Candidate | HCDR1 | ID NO | HCDR2 | NO | HCDR3 | NO |
| 139109 | NHGMS | 1051 | GIVYSGSTYYAA | 1091 | HGGESDV | 1131 |
| | | | SVKG | | | |
| 139103 | NYAMS | 1052 | GISRSGENTYYADS | 1092 | SPAHYYGGMDV | 1132 |
| | | | VKG | | | |
| 139105 | DYAMH | 1053 | GISWNSGSIGYADSV | 1093 | HSFLAY | 1133 |
| | | | KG | | | |
| 139111 | NHGMS | 1054 | GIVYSGSTYYAASV | 1094 | HGGESDV | 1134 |
| | | | KG | | | |
| 139100 | NFGIN | 1055 | WINPKNNNTNYAQ | 1095 | GPYYYQSYMDV | 1135 |
| | | | KFQG | | | |
| 139101 | SDAMT | 1056 | VISGSGGTTYYADS | 1096 | LDSSGYYYARGPRY | 1136 |
| | | | VKG | | | |
| 139102 | NYGIT | 1057 | WISAYNGNTNYAQ | 1097 | GPYYYYMDV | 1137 |
| | | | KFQG | | | |
| 139104 | NHGMS | 1058 | GIVYSGSTYYAASV | 1098 | HGGESDV | 1138 |
| | | | KG | | | |
| 139106 | NHGMS | 1059 | GIVYSGSTYYAASV | 1099 | HGGESDV | 1139 |
| | | | KG | | | |
| 139107 | NHGMS | 1060 | GIVYSGSTYYAASV | 1100 | HGGESDV | 1140 |
| | | | KG | | | |
| 139108 | DYYMS | 1061 | YISSSGSTIYYADSV | 1101 | ESGDGMDV | 1141 |
| | | | KG | | | |
| 139110 | DYYMS | 1062 | YISSSGNTIYYADSV | 1102 | STMVREDY | 1142 |
| | | | KG | | | |
| 139112 | NHGMS | 1063 | GIVYSGSTYYAASV | 1103 | HGGESDV | 1143 |
| | | | KG | | | |
| 139113 | NHGMS | 1064 | GIVYSGSTYYAASV | 1104 | HGGESDV | 1144 |
| | | | KG | | | |
| 139114 | NHGMS | 1065 | GIVYSGSTYYAASV | 1105 | HGGESDV | 1145 |
| | | | KG | | | |
| 149362 | SSYYYW | 1066 | SIYYSGSAYYNPSLK | 1106 | HWQEWPDAFDI | 1146 |
| | G | | S | | | |
| 149363 | TSGMCVS | 1067 | RIDWDEDKFYSTSL | 1107 | SGAGGTSATAFDI | 1147 |
| | | | KT | | | |
| 149364 | SYSMN | 1068 | SISSSSSYIYYADSVK | 1108 | TIAAVYAFDI | 1148 |
| | | | G | | | |
| 149365 | DYYMS | 1069 | YISSSGSTIYYADSV | 1109 | DLRGAFDI | 1149 |
| | | | KG | | | |
| 149366 | SHYIH | 1070 | MINPSGGVTAYSQT | 1110 | EGSGSGWYFDF | 1150 |
| | | | LQG | | | |
| 149367 | SGGYYW | 1071 | YIYYSGSTYYNPSLK | 1111 | AGIAARLRGAFDI | 1151 |
| | S | | S | | | |
| 149368 | SYAIS | 1072 | GIIPIFGTANYAQKF | 1112 | RGGYQLLRWDVGLL | 1152 |
| | | | QG | | RSAFDI | |
| 149369 | SNSAAW | 1073 | RTYYRSKWYSFYAI | 1113 | SSPEGLFLYWFDP | 1153 |
| | N | | SLKS | | | |
| BCMA_EBB- | SYAMS | 1074 | AISGSGGSTYYADS | 1114 | VEGSGSLDY | 1154 |
| C1978-A4 | | | VKG | | | |
| BCMA_EBB- | RYPMS | 1075 | GISDSGVSTYYADS | 1115 | RAGSEASDI | 1155 |
| C1978-G1 | | | AKG | | | |
| BCMA_EBB- | SYAMS | 1076 | AISGSGGSTYYADS | 1116 | ATYKRELRYYYGM | 1156 |
| C1979-C1 | | | VKG | | DV | |
| BCMA_EBB- | SYAMS | 1077 | AISGSGGSTYYADS | 1117 | ATYKRELRYYYGM | 1157 |
| C1978-C7 | | | VKG | | DV | |
| BCMA_EBB- | DYAMH | 1078 | GISWNSGSIGYADSV | 1118 | VGKAVPDV | 1158 |
| C1978- | | | KG | | | |
| D10 | | | | | | |
| BCMA_EBB- | DYAMH | 1079 | SINWKGNSLAYGDS | 1119 | HQGVAYYNYAMDV | 1159 |
| C1979- | | | VKG | | | |
| C12 | | | | | | |
| BCMA_EBB- | SYAMS | 1080 | AISGSGGSTYYADS | 1120 | VVRDGMDV | 1160 |
| C1980-G4 | VKG | | | | | |
| BCMA_EBB- | SYAMS | 1081 | AISGSGGSTYYADS | 1121 | IPQTGTFDY | 1161 |
| C1980-D2 | | | VKG | | | |
| BCMA_EBB- | SYAMS | 1082 | AISGSGGSTYYADS | 1122 | ANYKRELRYYYGM | 1162 |
| C1978- | | | VKG | | DV | |
| A10 | | | | | | |
| BCMA_EBB- | SYAMS | 1083 | AISGSGGSTYYADS | 1123 | ALVGATGAFDI | 1163 |
| C1978-D4 | | | VKG | | | |
| BCMA_EBB- | SYAMS | 1084 | AISGSGGSTYYADS | 1124 | WFGEGFDP | 1164 |
| C1980-A2 | | | VKG | | | |
| BCMA_EBB- | SYAMS | 1085 | AISGSGGSTYYADS | 1125 | VGYDSSGYYRDYYG | 1165 |
| C1981-C3 | | | VKG | | MDV | |
| BCMA_EBB- | SYAMS | 1086 | AISGSGGSTYYADS | 1126 | MGWSSGYLGAFDI | 1166 |
| C1978-G4 | | | VKG | | | |
| A7D12.2 | NFGMN | 1087 | WINTYTGESYFADD | 1127 | GEIYYGYDGGFAY | 1167 |
| | | | FKG | | | |
| C11D5.3 | DYSIN | 1088 | WINTETREPAYAYD | 1128 | DYSYAMDY | 1168 |
| | | | FRG | | | |
| C12A3.2 | HYSMN | 1089 | RINTESGVPIYADDF | 1129 | DYLYSLDF | 1169 |
| | | | KG | | | |
| C13F12.1 | HYSMN | 1090 | RINTETGEPLYADDF | 1130 | DYLYSCDY | 1170 |
| | | | KG | | | |

TABLE 4
Light Chain Variable Domain CDRs according to the Kabat numbering scheme
(Kabat et al. (1991), ″Sequences of Proteins of Immunological Interest,″
5th Ed. Public Health Service, National Institutes of Health, Bethesda, MD)

| | | SEQ ID | | SEQ ID | | SEQ ID |
|---|---|---|---|---|---|---|
| Candidate | LCDR1 | NO | LCDR2 | NO | LCDR3 | NO |
| 139109 | RASQSISSYLN | 1171 | AASSLQS | 1211 | QQSYSTPYT | 1251 |
| 139103 | RASQSISSSFLA | 1172 | GASRRAT | 1212 | QQYHSSPSWT | 1252 |
| 139105 | RSSQSLLHSNGYNYL | 1173 | LGSNRAS | 1213 | MQALQTPYT | 1253 |
| | D | | | | | |
| 139111 | KSSQSLLRNDGKTPL | 1174 | EVSNRFS | 1214 | MQNIQFPS | 1254 |
| | Y | | | | | |
| 139100 | RSSQSLLHSNGYNYL | 1175 | LGSKRAS | 1215 | MQALQTPYT | 1255 |
| | N | | | | | |
| 139101 | RASQSISSYLN | 1176 | GASTLAS | 1216 | QQSYKRAS | 1256 |
| 139102 | RSSQSLLYSNGYNYV | 1177 | LGSNRAS | 1217 | MQGRQFPYS | 1257 |
| | D | | | | | |
| 139104 | RASQSVSSNLA | 1178 | GASTRAS | 1218 | QQYGSSLT | 1258 |
| 139106 | RASQSVSSKLA | 1179 | GASIRAT | 1219 | QQYGSSSWT | 1259 |
| 139107 | RASQSVGSTNLA | 1180 | DASNRAT | 1220 | QQYGSSPPWT | 1260 |
| 139108 | RASQSISSYLN | 1181 | AASSLQS | 1221 | QQSYTLA | 1261 |
| 139110 | KSSESLVHNSGKTYL | 1182 | EVSNRDS | 1222 | MQGTHWPGT | 1262 |
| | N | | | | | |
| 139112 | QASEDINKFLN | 1183 | DASTLQT | 1223 | QQYESLPLT | 1263 |
| 139113 | RASQSVGSNLA | 1184 | GASTRAT | 1224 | QQYNDWLPVT | 1264 |
| 139114 | RASQSIGSSSLA | 1185 | GASSRAS | 1225 | QQYAGSPPFT | 1265 |
| 149362 | KASQDIDDAMN | 1186 | SATSPVP | 1226 | LQHDNFPLT | 1266 |
| 149363 | RASQDIYNNLA | 1187 | AANKSQS | 1227 | QHYYRFPYS | 1267 |
| 149364 | RSSQSLLHSNGYNYL | 1188 | LGSNRAS | 1228 | MQALQTPYT | 1268 |
| | D | | | | | |
| 149365 | GGNNIGTKSVH | 1189 | DDSVRPS | 1229 | QVWDSDSEHV | 1269 |
| | | | | | V | |
| 149366 | SGDGLSKKYVS | 1190 | RDKERPS | 1230 | QAWDDTTVV | 1270 |
| 149367 | RASQGIRNWLA | 1191 | AASNLQS | 1231 | QKYNSAPFT | 1271 |
| 149368 | GGNNIGSKSVH | 1192 | GKNNRPS | 1232 | SSRDSSGDHLR | 1272 |
| | | | | | V | |
| 149369 | QGDSLGNYYAT | 1193 | GTNNRPS | 1233 | NSRDSSGHHLL | 1273 |
| BCMA_EBB- | RASQSVSSAYLA | 1194 | GASTRAT | 1234 | QHYGSSFNGSS | 1274 |
| C1978-A4 | | | | | LFT | |
| BCMA_EBB- | RASQSVSNSLA | 1195 | DASSRAT | 1235 | QQFGTSSGLT | 1275 |
| C1978-G1 | | | | | | |
| BCMA_EBB- | RASQSVSSSFLA | 1196 | GASSRAT | 1236 | QQYHSSPSWT | 1276 |
| C1979-C1 | | | | | | |
| BCMA_EBB- | RASQSVSTTFLA | 1197 | GSSNRAT | 1237 | QQYHSSPSWT | 1277 |
| C1978-C7 | | | | | | |
| BCMA_EBB- | RASQSISSYLN | 1198 | AASSLQS | 1238 | QQSYSTPYS | 1278 |
| C1978-D10 | | | | | | |
| BCMA_EBB- | RATQSIGSSFLA | 1199 | GASQRAT | 1239 | QHYESSPSWT | 1279 |
| C1979-C12 | | | | | | |
| BCMA_EBB- | RASQSVSSSYLA | 1200 | GASSRAT | 1240 | QQYGSPPRFT | 1280 |
| C1980-G4 | | | | | | |
| BCMA_EBB- | RASQSVSSSYLA | 1201 | GASSRAT | 1241 | QHYGSSPSWT | 1281 |
| C1980-D2 | | | | | | |
| BCMA_EBB- | RASQRVASNYLA | 1202 | GASSRAT | 1242 | QHYDSSPSWT | 1282 |
| C1978-A10 | | | | | | |
| BCMA_EBB- | RASQSLSSNFLA | 1203 | GASNWAT | 1243 | QYYGTSPMYT | 1283 |
| C1978-D4 | | | | | | |
| BCMA_EBB- | RSSQSLLHSNGYNYL | 1204 | LGSNRAS | 1244 | MQALQTPLT | 1284 |
| C1980-A2 | D | | | | | |
| BCMA_EBB- | RASQSVSSSYLA | 1205 | GTSSRAT | 1245 | QHYGNSPPKFT | 1285 |
| C1981-C3 | | | | | | |
| BCMA_EBB- | RASQSVASSFLA | 1206 | GASGRAT | 1246 | QHYGGSPRLT | 1286 |
| C1978-G4 | | | | | | |
| A7D12.2 | RASQDVNTAVS | 1207 | SASYRYT | 1247 | QQHYSTPWT | 1287 |
| C11D5.3 | RASESVSVIGAHLIH | 1208 | LASNLET | 1248 | LQSRIFPRT | 1288 |
| C12A3.2 | RASESVTILGSHLIY | 1209 | LASNVQT | 1249 | LQSRTIPRT | 1289 |
| C13F12.1 | RASESVTILGSHLIY | 1210 | LASNVQT | 1250 | LQSRTIPRT | 1290 |

TABLE 5
Additional exemplary BCMA CAR sequences

| Name | SEQ ID NO: |
|---|---|
| A7D12.2 VH | 255 |
| A7D12.2 VL | 259 |
| A7D12.2 scFv domain | 263 |
| A7D12.2 Full CART | 267 |
| C11D5.3 VH | 256 |
| C11D5.3 VL | 260 |
| C11D5.3 scFv domain | 264 |
| C11D5.3 Full CART | 268 |
| C12A3.2 VH | 257 |
| C12A3.2 VL | 261 |
| C12A3.2 scFv domain | 265 |
| C12A3.2 Full CART | 269 |
| C13F12.1 VH | 258 |
| C13F12.1 VL | 262 |
| C13F12.1 scFv domain | 266 |
| C13F12.1 Full CART | 270 |

### RNA Transfection

Disclosed herein are methods for producing an in vitro transcribed RNA CAR. The present invention also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases (SEQ ID NO: 2025) in length. RNA so produced can efficiently transfect different kinds of cells. In one aspect, the template includes sequences for the CAR.

In one aspect the anti-BCMA CAR is encoded by a messenger RNA (mRNA). In one aspect the mRNA encoding the anti-BCMA CAR is introduced into an immune effector cell, e.g., a T cell or a NK cell, for production of a CAR-expressing cell (e.g., CART cell or CAR-expressing NK cell).

In one embodiment, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR of the present invention. For example, the template for the RNA CAR comprises an extracellular region comprising a single chain variable domain of an anti-tumor antibody; a hinge region, a transmembrane domain (e.g., a transmembrane domain of CD8a); and a cytoplasmic region that includes an intracellular signaling domain, e.g., comprising the signaling domain of CD3-zeta and the signaling domain of 4-1BB.

In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 2026) (size can be 50-5000 T (SEQ ID NO: 2027)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines (SEQ ID NO: 2028).

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides (SEQ ID NO: 2024) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1(2011):R14-20; Singh et al. Cancer Res. 15(2008):2961-2971; Huang et al. Mol. Ther. 16(2008):580-589; Grabundzija et al. Mol. Ther. 18(2010):1200-1209; Kebriaei et al. Blood. 122.21(2013):166; Williams Molecular Therapy 16.9(2008):1515-16; Bell et al. Nat. Protoc. 2.12(2007):3153-65; and Ding et al. Cell. 122.3(2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. supra.

Exemplary transposons include a pT2-based transposon. See, e.g., Grabundzija et al. Nucleic Acids Res. 41.3(2013):1829-47; and Singh et al. Cancer Res. 68.8(2008): 2961-2971, all of which are incorporated herein by reference. Exemplary transposases include a Tcl/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a CAR described herein. Provided herein are methods of generating a cell, e.g., T cell or NK cell, that stably expresses a CAR described herein, e.g., using a transposon system such as SBTS.

In accordance with methods described herein, in some embodiments, one or more nucleic acids, e.g., plasmids, containing the SBTS components are delivered to a cell (e.g., T or NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR described herein. In some embodiments, the nucleic acid contains a transposon comprising a transgene (e.g., a nucleic acid encoding a CAR described herein) as well as a nucleic acid sequence encoding a transposase enzyme. In other embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Nucleic Acid Constructs Encoding a CAR

The present invention also provides nucleic acid molecules encoding one or more CAR constructs described herein. In one aspect, the nucleic acid molecule is provided as a messenger RNA transcript. In one aspect, the nucleic acid molecule is provided as a DNA construct.

Accordingly, in one aspect, the invention pertains to an isolated nucleic acid molecule encoding a chimeric antigen receptor (CAR), wherein the CAR comprises an antigen binding domain, a transmembrane domain, and an intracellular signaling domain comprising a stimulatory domain, e.g., a costimulatory signaling domain and/or a primary signaling domain, e.g., zeta chain.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, CRISPR, CAS9, and zinc finger nucleases. See below June et al. 2009 Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009).

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired. The nucleotide sequences of exemplary PGK promoters are provided below.

### WT PGK Promoter

(SEQ ID NO: 1291)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCGCCTCGTCCTTCGCAGCGGC
CCCCCGGGTGTTCCCATCGCCGCTTCTAGGCCCACTGCGACGCTTGCCT
GCACTTCTTACACGCTCTGGGTCCCAGCCGCGGCGACGCAAAGGGCCTT
GGTGCGGGTCTCGTCGGCGCAGGGACGCGTTTGGGTCCCGACGGAACCT
TTTCCGCGTTGGGGTTGGGGCACCATAAGCT

### Exemplary Truncated PGK Promoters:

PGK100:
(SEQ ID NO: 1292)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:
(SEQ ID NO: 1293)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:
(SEQ ID NO: 1294)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:
(SEQ ID NO: 1295)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one embodiment, the vector can further comprise a nucleic acid encoding a second CAR. In one embodiment, the second CAR includes an antigen binding domain to a target expressed on acute myeloid leukemia cells, such as, e.g., CD123, CD34, CLL-1, folate receptor beta, or FLT3; or a target expressed on a B cell, e.g., CD10, CD19, CD20, CD22, CD34, CD123, FLT-3, ROR1, CD79b, CD179b, or CD79a. In one embodiment, the vector comprises a nucleic acid sequence encoding a first CAR that specifically binds a first antigen and includes an intracellular signaling domain having a costimulatory signaling domain but not a primary signaling domain, and a nucleic acid encoding a second CAR that specifically binds a second, different, antigen and includes an intracellular signaling domain having a primary signaling domain but not a costimulatory signaling domain. In one embodiment, the vector comprises a nucleic acid encoding a first BCMA CAR that includes a BCMA binding domain, a transmembrane domain and a costimulatory domain and a nucleic acid encoding a second CAR that targets an antigen other than BCMA (e.g., an antigen expressed on AML cells, e.g., CD123, CD34, CLL-1, folate receptor beta, or FLT3; or an antigen expressed on a B cell, e.g., CD10, CD19, CD20, CD22, CD34, CD123, FLT-3, ROR1, CD79b, CD179b, or CD79a) and includes an antigen binding domain, a transmembrane domain and a primary signaling domain. In another embodiment, the vector comprises a nucleic acid encoding a first BCMA CAR that includes a BCMA binding domain, a transmembrane domain and a primary signaling domain and a nucleic acid encoding a second CAR that specifically binds an antigen other than BCMA (e.g., an antigen expressed on AML cells, e.g., CD123, CD34, CLL-1, folate receptor beta, or FLT3; or an antigen expressed on a B cell, e.g., CD10, CD19, CD20, CD22, CD34, CD123, FLT-3, ROR1, CD79b, CD179b, or CD79a) and includes an antigen binding domain to the antigen, a transmembrane domain and a costimulatory signaling domain.

In one embodiment, the vector comprises a nucleic acid encoding a BCMA CAR described herein and a nucleic acid encoding an inhibitory CAR. In one embodiment, the inhibitory CAR comprises an antigen binding domain that binds an antigen found on normal cells but not cancer cells, e.g., normal cells that also express BCMA. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGFR beta.

In embodiments, the vector may comprise two or more nucleic acid sequences encoding a CAR, e.g., a BCMA CAR described herein and a second CAR, e.g., an inhibitory CAR or a CAR that specifically binds to an antigen other than BCMA (e.g., an antigen expressed on AML cells, e.g., CD123, CLL-1, CD34, FLT3, or folate receptor beta; or antigen expression B cells, e.g., CD10, CD19, CD20, CD22, CD34, CD123, FLT-3, ROR1, CD79b, CD179b, or CD79a). In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include the following, wherein the GSG residues are optional:

T2A:
(SEQ ID NO: 1296)
(GSG)EGRGSLLTCGDVEENPGP
P2A:
(SEQ ID NO: 1297)
(GSG)ATNFSLLKQAGDVEENPGP
E2A:
(SEQ ID NO: 1298)
(GSG)QCTNYALLKLAGDVESNPGP
F2A:
(SEQ ID NO: 1299)
(GSG)VKQTLNFDLLKLAGDVESNPGP

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising a CAR encoding nucleic acid molecule. In one aspect, a CAR vector can be directly transduced into a cell, e.g., a T cell or NK cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian T cells or NK cells. In one aspect, the mammalian T cell is a human T cell. In one aspect, the mammalian NK cell is a human NK cell.

### Sources of Cells

Prior to expansion and genetic modification, a source of cells, e.g., immune effector cells (e.g., T cells or NK cells), is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present invention, any number of immune effector cell (e.g., T cell or NK cell) lines available in the art, may be used. In certain aspects of the present invention, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD4+, CD8+, CD45RA+, and/or CD45RO+ T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. In certain aspects, it may be desirable to enrich for cells that are CD127low. Alternatively, in certain aspects, T regulatory cells are depleted by anti-C25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is le7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing TREG cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGFR beta. In embodiments, the checkpoint inhibitor is PD1 or PD-L1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

In one embodiment, a T cell population can be selected that expresses one or more of IFN-γ, TNFα, IL-17A, IL-2, IL-3, IL-4, GM-CSF, IL-10, IL-13, granzyme B, and perform, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/ml is used. In one aspect, a concentration of 1 billion cells/ml is used. In a further aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10e6/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as immune effector cells, e.g., T cells or NK cells, isolated and frozen for later use in cell therapy, e.g., T cell therapy, for any number of diseases or conditions that would benefit from cell therapy, e.g., T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the immune effector cells (e.g., T cells or NK cells) may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Modifications of CAR Cells, Including Allogeneic CAR Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II, and/or beta-2 microglobulin (β₂m). Compositions of allogeneic CAR and methods thereof have been described in, e.g., pages 227-237 of WO 2016/014565, incorporated herein by reference in its entirety.

In some embodiments, a cell, e.g., a T cell or a NK cell, is modified to reduce the expression of a TCR, and/or HLA, and/or β₂m, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGFR beta), using, e.g., a method described herein, e.g., siRNA, shRNA, clustered regularly interspaced short palindromic repeats (CRISPR) transcription-activator like effector nuclease (TALEN), or zinc finger endonuclease (ZFN).

In some embodiments, a cell, e.g., a T cell or a NK cell is engineered to express a telomerase subunit, e.g., the catalytic subunit of telomerase, e.g., TERT, e.g., hTERT. In one embodiment, such modification improves persistence of the cell in a patient.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al, J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain aspects of the invention, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells, e.g., a BCMA CAR cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a BCMA CAR cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a BCMA CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a BCMA CAR cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

In one aspect of the present invention, the mixture may be cultured for several hours (about 3 hours) to about 14 days or any hourly integer value in between. In one aspect, the mixture may be cultured for 21 days. In one aspect of the invention the beads and the T cells are cultured together for about eight days. In one aspect, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence of IL-15 and/or IL-7 (e.g., IL-15 and IL-7).

In embodiments, methods described herein, e.g., CAR-expressing cell manufacturing methods, comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Rα) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population. Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a BCMA CAR is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a BCMA CAR are described in further detail below

Western blot analysis of CAR expression in primary T cells can be used to detect the presence of monomers and dimers. See, e.g., Milone et al., Molecular Therapy 17(8): 1453-1464 (2009). Very briefly, T cells (1:1 mixture of CD4⁺ and CD8⁺ T cells) expressing the CARs are expanded in vitro for more than 10 days followed by lysis and SDS-PAGE under reducing conditions. CARs containing the full length TCR-ζ cytoplasmic domain and the endogenous TCR-ζ chain are detected by western blotting using an antibody to the TCR-ζ chain. The same T cell subsets are used for SDS-PAGE analysis under non-reducing conditions to permit evaluation of covalent dimer formation.

In vitro expansion of CAR⁺ T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with BCMA-expressing cells, such as multiple myeloma cell lines or K562-BCMA, following washing. Exogenous IL-2 is added to the cultures every other day at 100 IU/ml. GFP⁺ T cells are enumerated by flow cytometry using bead-based counting. See, e.g., Milone et al., Molecular Therapy 17(8): 1453-1464 (2009).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CART activity. For example, xenograft model using human BCMA-specific CAR⁺ T cells to treat a primary human multiple myeloma in immunodeficient mice can be used. See, e.g., Milone et al., Molecular Therapy 17(8): 1453-1464 (2009). Very briefly, after establishment of MM, mice are randomized as to treatment groups. Different numbers of BCMA CART cells can be injected into immunodeficient mice bearing MM. Animals are assessed for disease progression and tumor burden at weekly intervals. Survival curves for the groups are compared using the log-rank test. In addition, absolute peripheral blood CD4⁺ and CD8⁺ T cell counts 4 weeks following T cell injection in the immunodeficient mice can also be analyzed. Mice are injected with multiple myeloma cells and 3 weeks later are injected with T cells engineered to express BCMA CAR, e.g., by a bicistronic lentiviral vector that encodes the CAR linked to eGFP. T cells are normalized to 45-50% input GFP⁺ T cells by mixing with mock-transduced cells prior to injection, and confirmed by flow cytometry. Animals are assessed for leukemia at 1-week intervals. Survival curves for the CAR⁺ T cell groups are compared using the log-rank test.

Assessment of cell proliferation and cytokine production has been previously described, e.g., at Milone et al., Molecular Therapy 17(8): 1453-1464 (2009). Briefly, assessment of CAR-mediated proliferation is performed in microtiter plates by mixing washed T cells with K562 cells expressing BCMA or other BCMA-expressing myeloma cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8⁺ T cell expansion ex vivo. T cells are enumerated in cultures using CountBright™ fluorescent beads (Invitrogen, Carlsbad, Calif.) and flow cytometry as described by the manufacturer. CAR⁺ T cells are identified by GFP expression using T cells that are engineered with eGFP-2A linked CAR-expressing lentiviral vectors. For CAR+ T cells not expressing GFP, the CAR+ T cells are detected with biotinylated recombinant BCMA protein and a secondary avidin-PE conjugate. CD4+ and CD8⁺ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences, San Diego, Calif.) according the manufacturer's instructions. Fluorescence is assessed using a FACScalibur flow cytometer, and data is analyzed according to the manufacturer's instructions.

Cytotoxicity can be assessed by a standard 51Cr-release assay. See, e.g., Milone et al., Molecular Therapy 17(8): 1453-1464 (2009). Briefly, target cells (e.g., K562 lines expressing BCMA and primary multiple myeloma cells) are loaded with 51Cr (as NaCrO4, New England Nuclear, Boston, Mass.) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released 51Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average 51Cr released for each experimental condition. Alternatively, cytotoxicity can also be assessed using a Bright-Glo™ Luciferase Assay.

Imaging technologies can be used to evaluate specific trafficking and proliferation of CARs in tumor-bearing animal models. Such assays have been described, for example, in Barrett et al., Human Gene Therapy 22:1575-1586 (2011). Briefly, NOD/SCID/γc^{-/-} (NSG) mice or other immunodeficient are injected IV with multiple myeloma cells followed 7 days later with BCMA CART cells 4 hour after electroporation with the CAR constructs. The T cells are stably transfected with a lentiviral construct to express firefly luciferase, and mice are imaged for bioluminescence. Alternatively, therapeutic efficacy and specificity of a single injection of CAR⁺ T cells in a multiple myeloma xenograft model can be measured as the following: NSG mice are injected with multiple myeloma cells transduced to stably express firefly luciferase, followed by a single tail-vein injection of T cells electroporated with BCMA CAR construct days later. Animals are imaged at various time points post injection. For example, photon-density heat maps of firefly luciferasepositive tumors in representative mice at day 5 (2 days before treatment) and day 8 (24 hr post CAR+PBLs) can be generated.

Alternatively, or in combination to the methods disclosed herein, methods and compositions for one or more of: detection and/or quantification of CAR-expressing cells (e.g., in vitro or in vivo (e.g., clinical monitoring)); immune cell expansion and/or activation; and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain) In other embodiments, the CAR ligand is a CAR antigen molecule (e.g., a CAR antigen molecule as described herein).

In one aspect, a method for detecting and/or quantifying CAR-expressing cells is disclosed. For example, the CAR ligand can be used to detect and/or quantify CAR-expressing cells in vitro or in vivo (e.g., clinical monitoring of CAR-expressing cells in a patient, or dosing a patient). The method includes:

providing the CAR ligand (optionally, a labelled CAR ligand, e.g., a CAR ligand that includes a tag, a bead, a radioactive or fluorescent label);

acquiring the CAR-expressing cell (e.g., acquiring a sample containing CAR-expressing cells, such as a manufacturing sample or a clinical sample);

contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In another aspect, a method of expanding and/or activating cells (e g, immune effector cells) is disclosed. The method includes:

providing a CAR-expressing cell (e.g., a first CAR-expressing cell or a transiently expressing CAR cell);

contacting said CAR-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet another aspect, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference. In one embodiment, the anti-idiotypic antibody molecule recognizes an anti-CD19 antibody molecule, e.g., an anti-CD19 scFv. For instance, the anti-idiotypic antibody molecule can compete for binding with the CD19-specific CAR mAb clone no. 136.20.1 described in Jena et al., PLOS March 2013 8:3 e57838; may have the same CDRs (e.g., one or more of, e.g., all of, VH CDR1, VH CDR2, CH CDR3, VL CDR1, VL CDR2, and VL CDR3, using the Kabat definition, the Chothia definition, or a combination of the Kabat and Chothia definitions) as the CD19-specific CAR mAb clone no. 136.20.1; may have one or more (e.g., 2) variable regions as the CD19-specific CAR mAb clone no. 136.20.1, or may comprise the CD19-specific CAR mAb clone no. 136.20.1. In some embodiments, the anti-idiotypic antibody was made according to a method described in Jena et al. In another embodiment, the anti-idiotypic antibody molecule is an anti-idiotypic antibody molecule described in WO 2014/190273. In some embodiments, the anti-idiotypic antibody molecule has the same CDRs (e.g., one or more of, e.g., all of, VH CDR1, VH CDR2, CH CDR3, VL CDR1, VL CDR2, and VL CDR3) as an antibody molecule of WO 2014/190273 such as 136.20.1; may have one or more (e.g., 2) variable regions of an antibody molecule of WO 2014/190273, or may comprise an antibody molecule of WO 2014/190273 such as 136.20.1. In other embodiments, the anti-CAR antibody binds to a constant region of the extracellular binding domain of the CAR molecule, e.g., as described in WO 2014/190273. In some embodiments, the anti-CAR antibody binds to a constant region of the extracellular binding domain of the CAR molecule, e.g., a heavy chain constant region (e.g., a CH2-CH3 hinge region) or light chain constant region. For instance, in some embodiments the anti-CAR antibody competes for binding with the 2D3 monoclonal antibody described in WO 2014/190273, has the same CDRs (e.g., one or more of, e.g., all of, VH CDR1, VH CDR2, CH CDR3, VL CDR1, VL CDR2, and VL CDR3) as 2D3, or has one or more (e.g., 2) variable regions of 2D3, or comprises 2D3 as described in WO 2014/190273.

In some aspects and embodiments, the compositions and methods herein are optimized for a specific subset of T cells, e.g., as described in U.S. Ser. No. 62/031,699 filed Jul. 31, 2014, the contents of which are incorporated herein by reference in their entirety. In some embodiments, the optimized subsets of T cells display an enhanced persistence compared to a control T cell, e.g., a T cell of a different type (e.g., CD8⁺ or CD4⁺) expressing the same construct.

In some embodiments, a CD4⁺ T cell comprises a CAR described herein, which CAR comprises an intracellular signaling domain suitable for (e.g., optimized for, e.g., leading to enhanced persistence in) a CD4⁺ T cell, e.g., an ICOS domain. In some embodiments, a CD8⁺ T cell comprises a CAR described herein, which CAR comprises an intracellular signaling domain suitable for (e.g., optimized for, e.g., leading to enhanced persistence of) a CD8⁺ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain that targets BCMA).

In an aspect, described herein is a method of treating a subject, e.g., a subject having cancer. The method includes administering to said subject, an effective amount of:

1) a CD4⁺ T cell comprising a CAR (the CAR^{CD4+})

comprising:

an antigen binding domain, e.g., an antigen binding domain described herein, e.g., an antigen binding domain that targets BCMA;

a transmembrane domain; and

an intracellular signaling domain, e.g., a first costimulatory domain, e.g., an ICOS domain; and

2) a CD8⁺ T cell comprising a CAR (the CAR^{CD8+}) comprising:

an antigen binding domain, e.g., an antigen binding domain described herein, e.g., an antigen binding domain that targets BCMA;

a transmembrane domain; and

an intracellular signaling domain, e.g., a second costimulatory domain, e.g., a 4-1BB domain, a CD28 domain, or another costimulatory domain other than an ICOS domain;

wherein the CAR^{CD4+} and the CAR^{CD8+} differ from one another.

Optionally, the method further includes administering:

3) a second CD8+ T cell comprising a CAR (the second CAR^{CD8+}) comprising:

an antigen binding domain, e.g., an antigen binding domain described herein, e.g., an antigen binding domain that specifically binds BCMA;

a transmembrane domain; and

an intracellular signaling domain, wherein the second CAR″ comprises an intracellular signaling domain, e.g., a costimulatory signaling domain, not present on the CAR″, and, optionally, does not comprise an ICOS signaling domain

Other assays, including those that are known in the art can also be used to evaluate the BCMA CAR constructs of the invention.

### Therapeutic Application

BCMA Associated Diseases and/or Disorders

In one aspect, the invention provides methods for treating a disease associated with BCMA expression. In one aspect, the invention provides methods for treating a disease wherein part of the tumor is negative for BCMA and part of the tumor is positive for BCMA For example, the CAR of the invention is useful for treating subjects that have undergone treatment for a disease associated with elevated expression of BCMA, wherein the subject that has undergone treatment for elevated levels of BCMA exhibits a disease associated with elevated levels of BCMA. In embodiments, the CAR of the invention is useful for treating subjects that have undergone treatment for a disease associated with expression of BCMA, wherein the subject that has undergone treatment related to expression of BCMA exhibits a disease associated with expression of BCMA.

In one embodiment, the invention provides methods for treating a disease wherein BCMA is expressed on both normal cells and cancers cells, but is expressed at lower levels on normal cells. In one embodiment, the method further comprises selecting a CAR that binds of the invention with an affinity that allows the BCMA CAR to bind and kill the cancer cells expressing BCMA but less than 30%, 25%, 20%, 15%, 10%, 5% or less of the normal cells expressing BCMA are killed, e.g., as determined by an assay described herein. For example, a killing assay such as flow cytometry based on Cr51 CTL can be used. In one embodiment, the BCMA CAR has an antigen binding domain that has a binding affinity KD of 10⁻⁴ M to 10⁻⁸ M, e.g., 10⁻⁵ M to 10⁻⁷ M, e.g., 10⁻⁶ M or 10⁻⁷ M, for the target antigen. In one embodiment, the BCMA antigen binding domain has a binding affinity that is at least five-fold, 10-fold, 20-fold, 30-fold, 50-fold, 100-fold or 1,000-fold less than a reference antibody, e.g., an antibody described herein.

In one aspect, the invention pertains to a vector comprising BCMA CAR operably linked to promoter for expression in mammalian immune effector cells, e.g., T cells or NK cells. In one aspect, the invention provides a recombinant immune effector cell, e.g., T cell or NK cell, expressing the BCMA CAR for use in treating BCMA-expressing tumors, wherein the recombinant immune effector cell (e.g., T cell or NK cell) expressing the BCMA CAR is termed a BCMA CAR-expressing cell (e.g., BCMA CART or BCMA CAR-expressing NK cell). In one aspect, the BCMA CAR-expressing cell (e.g., BCMA CART or BCMA CAR-expressing NK cell) of the invention is capable of contacting a tumor cell with at least one BCMA CAR of the invention expressed on its surface such that the BCMA CAR-expressing cell (e.g., BCMA CART or BCMA CAR-expressing NK cell)targets the tumor cell and growth of the tumor is inhibited.

In one aspect, the invention pertains to a method of inhibiting growth of a BCMA-expressing tumor cell, comprising contacting the tumor cell with a BCMA CAR-expressing cell (e.g., BCMA CART or BCMA CAR-expressing NK cell) of the present invention such that the BCMA CAR-expressing cell (e.g., BCMA CART or BCMA CAR-expressing NK cell) is activated in response to the antigen and targets the cancer cell, wherein the growth of the tumor is inhibited.

In one aspect, the invention pertains to a method of treating cancer in a subject. The method comprises administering to the subject a BCMA CAR-expressing cell (e.g., BCMA CART or BCMA CAR-expressing NK cell) of the present invention such that the cancer is treated in the subject. An example of a cancer that is treatable by the BCMA CAR-expressing cell (e.g., BCMA CART or BCMA CAR-expressing NK cell) of the invention is a cancer associated with expression of BCMA.

The invention includes a type of cellular therapy where immune effector cells (e.g., T cells or NK cells) are genetically modified to express a chimeric antigen receptor (CAR) and the BCMA CAR-expressing cell (e.g., BCMA CART or BCMA CAR-expressing NK cell) is infused to a recipient in need thereof. The infused cell is able to kill tumor cells in the recipient. Unlike antibody therapies, CAR-modified cells, e.g., T cells or NK cells, are able to replicate in vivo resulting in long-term persistence that can lead to sustained tumor control. In various aspects, the cells (e.g., T cells or NK cells) administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the cell (e.g., T cell or NK cell) to the patient.

The invention also includes a type of cellular therapy where immune effector cells (e.g., T cells or NK cells) are modified, e.g., by in vitro transcribed RNA, to transiently express a chimeric antigen receptor (CAR) and the immune effector cell (e.g., T cell or NK cell) is infused to a recipient in need thereof. The infused cell is able to kill tumor cells in the recipient. Thus, in various aspects, the immune effector cells (e.g., T cells or NK cells) administered to the patient, is present for less than one month, e.g., three weeks, two weeks, one week, after administration of the immune effector cell (e.g., T cell or NK cell) to the patient.

Without wishing to be bound by any particular theory, the anti-tumor immunity response elicited by the CAR-modified immune effector cells (e.g., T cells or NK cells) may be an active or a passive immune response, or alternatively may be due to a direct vs indirect immune response. In one aspect, the CAR transduced immune effector cells (e.g., T cells or NK cells) exhibit specific proinflammatory cytokine secretion and potent cytolytic activity in response to human cancer cells expressing the BCMA, resist soluble BCMA inhibition, mediate bystander killing and mediate regression of an established human tumor. For example, antigen-less tumor cells within a heterogeneous field of BCMA-expressing tumor may be susceptible to indirect destruction by BCMA-redirected immune effector cells (e.g., T cells or NK cells) that has previously reacted against adjacent antigen-positive cancer cells.

In one aspect, the fully-human CAR-modified immune effector cells (e.g., T cells or NK cells) of the invention may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a CAR to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the CAR-modified immune effector cells (e.g., T cells or NK cells) of the invention are used in the treatment of diseases, disorders and conditions associated with expression of BCMA. In certain aspects, the cells of the invention are used in the treatment of patients at risk for developing diseases, disorders and conditions associated with expression of BCMA. Thus, the present invention provides methods for the treatment or prevention of diseases, disorders and conditions associated with expression of BCMA comprising administering to a subject in need thereof, a therapeutically effective amount of the CAR-modified immune effector cells (e.g., T cells or NK cells) of the invention.

In one aspect the CAR-expressing cells (e.g., CART cells or CAR-expressing NK cells) of the inventions may be used to treat a proliferative disease such as a cancer or malignancy or is a precancerous condition such as a myelodysplasia, a myelodysplastic syndrome or a preleukemia. In one aspect, the cancer is a hematolical cancer. Hematological cancer conditions are the types of cancer such as leukemia and malignant lymphoproliferative conditions that affect blood, bone marrow and the lymphatic system. In one aspect, the hematological cancer is a leukemia or a hematological. An example of a disease or disorder associated with BCMA is multiple myeloma (also known as MM) (See Claudio et al., Blood. 2002, 100(6):2175-86; and Novak et al., Blood. 2004, 103(2):689-94). Multiple myeloma, also known as plasma cell myeloma or Kahler's disease, is a cancer characterized by an accumulation of abnormal or malignant plasma B-cells in the bone marrow. Frequently, the cancer cells invade adjacent bone, destroying skeletal structures and resulting in bone pain and fractures. Most cases of myeloma also features the production of a paraprotein (also known as M proteins or myeloma proteins), which is an abnormal immunoglobulin produced in excess by the clonal proliferation of the malignant plasma cells. Blood serum paraprotein levels of more than 30 g/L is diagnostic of multiple myeloma, according to the diagnostic criteria of the International Myeloma Working Group (IMWG) (See Kyle et al. (2009), Leukemia. 23:3-9). Other symptoms or signs of multiple myeloma include reduced kidney function or renal failure, bone lesions, anemia, hypercalcemia, and neurological symptoms.

Criteria for distinguishing multiple myeloma from other plasma cell proliferative disorders have been established by the International Myeloma Working Group (See Kyle et al. (2009), Leukemia. 23:3-9). All three of the following criteria must be met:
Clonal bone marrow plasma cells ≥10%
Present of serum and/or urinary monoclonal protein (except in patients with true non-secretory multiple myeloma)
Evidence of end-organ damage attributable to the underlying plasma cell proliferative disorder, specifically:
   Hypercalcemia: serum calcium ≥11.5 mg/100 ml
   Renal insufficienty: serum creatinine >1.73 mmol/1
   Anemia: normochromic, normocytic with a hemoglobin value of >2 g/100 ml below the lower limit of normal, or a hemoglobin value <10 g/100 ml
   Bone lesions: lytic lesions, severe osteopenia, or pathologic fractures.

Other plasma cell proliferative disorders that can be treated by the compositions and methods described herein include, but are not limited to, asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

Two staging systems are used in the staging of multiple myeloma: the International Staging System (ISS) (See Greipp et al. (2005), J. Clin. Oncol. 23 (15):3412-3420, herein incorporated by reference in its entirety) and the Durie-Salmon Staging system (DSS) (See Durie et al. (1975), Cancer 36 (3): 842-854, herein incorporated by reference in its entirety). The two staging systems are summarized in the table below:

TABLE 6
Staging systems for the staging of multiple myeloma

| | International Staging System | | Durie-Salmon Staging System | |
|---|---|---|---|---|
| | | Median | | Median |
| Stage | Criteria | survival | Criteria | survival* |
| I | β₂M <3.5 mg/l and | 62 months | All of the following: | IA: 62 months |
| | serum albumin ≥3.5 g/dL | | Hemoglobin level >10 g/dL | |
| | | | Serum calcium, normal or <12 mg/dL | IB: 22 months |
| | | | Bone x-ray, normal or 1 | |
| | | | plasmacytoma only | |
| | | | Low monoclonal protein | |
| | | | production (IgG <5 g/dL, | |
| | | | IgA <3 g/dL, Bence Jones protein <4 | |
| | | | g/dL per 24 hours | |
| II | Neither stage I or stage III | 44 months | Neither stage I or stage III | IIA: 58 months |
| | | | | IIB: 354 months |
| III | β₂M ≥5.5 mg/l | 29 months | One or more of the following: | IIIA: 45 months |
| | | | Hemogloblin level <8.5 g/dL | IIIB: 24 months |
| | | | Serum calcium, normal or >12 mg/dL | |
| | | | Advanced osteolytic lesions | |
| | | | High monoclonal protein | |
| | | | production (IgG >7 g/dL, | |
| | | | IgA >5 g/dL, Bence Jones protein >12 | |
| | | | g/dL per 24 hours | |

*The Durie-Salmon Staging system also includes a subclassification that designates the status of renal function. The designation of “A” or “B” is added after the stage number, wherein “A” indicates relatively normal renal function (serum creatinine value <2.0 mg/dL), and B indicates abnormal renal function (serum creatinine value >2.0 mg/dL).

A third staging system for multiple myeloma is referred to as Revised International Staging System (R-ISS) (see Palumbo A, Avet-Loiseau H, Oliva S, et al. Journal of clinical oncology: official journal of the American Society of Clinical Oncology 2015; 33:2863-9, herein incorporated by reference in its entirety). R-ISS stage I includes ISS stage I (serum β2-microglobulin level <3.5 mg/L and serum albumin level ≥3.5 g/dL), no high-risk CA [del(17p) and/or t(4; 14) and/or t(14; 16)], and normal LDH level (less than the upper limit of normal range). R-ISS stage III includes ISS stage III (serum J32-microglobulin level ≥5.5 mg/L) and high-risk CA or high LDH level. R-ISS stage II includes all the other possible combinations.

The response of patients can be determined based on IMWG 2016 criteria, as disclosed in Kumar S, Paiva B, Anderson K C, et al. International Myeloma Working Group consensus criteria for response and minimal residual disease assessment in multiple myeloma. The Lancet Oncology; 17(8):e328-e346 (2016), herein incorporated by reference in its entirety. Table 7 provides IMWG 2016 criteria for response assessment.

TABLE 7
IMWG criteria for response assessment including criteria for minimal residual disease (MRD)

Response criteria*

IMWG MRD criteria (requires a complete response as defined below)

| | |
|---|---|
| Sustained MRD-negative | MRD negativity in the marrow (NGF or NGS, or both) and by imaging |
| | as defined below, confirmed minimum of 1 year apart. Subsequent |
| | evaluations can be used to further specify the duration of negativity (e.g., |
| | MRD-negative at 5 years)† |
| Flow MRD-negative | Absence of phenotypically aberrant clonal plasma cells by NGF‡ on bone |
| | marrow aspirates using the EuroFlow standard operation procedure for |
| | MRD detection in multiple myeloma (or validated equivalent method) |
| | with a minimum sensitivity of 1 in 10⁵ nucleated cells or higher |
| Sequencing MRD- | Absence of clonal plasma cells by NGS on bone marrow aspirate in |
| negative | which presence of a clone is defined as less than two identical sequencing |
| | reads obtained after DNA sequencing of bone marrow aspirates using the |
| | LymphoSIGHT platform (or validated equivalent method) with a |
| | minimum sensitivity of 1 in 10⁵ nucleated cells§ or higher |
| Imaging plus MRD- | MRD negativity as defined by NGF or NGS plus disappearance of every |
| negative | area of increased tracer uptake found at baseline or a preceding PET/CT |
| | or decrease to less mediastinal blood pool SUV or decrease to less than |
| | that of surrounding normal tissue¶ |

Standard IMWG response criteria||

| | |
|---|---|
| Stringent complete | Complete response as defined below plus normal FLC ratio** and |
| response | absence of clonal cells in bone marrow biopsy by immunohistochemistry |
| | (κ/λ ratio ≤4:1 or ≥1:2 for |
| | κ and λ patients, respectively, after counting ≥100 plasma cells)†† |
| Complete response | Negative immunofixation on the serum and urine and disappearance of |
| | any soft tissue plasmacytomas and ≤5% plasma cells in bone marrow |
| | aspirates |
| Very good partial | Serum and urine M-protein detectable by immunofixation but not on |
| response | electrophoresis or ≥90% reduction in serum M-protein plus urine M- |
| | protein level ≤100 mg per 24 h |
| Partial response | ≥50% reduction of serum M-protein plus reduction in 24 h urinary M- |
| | protein by ≥90% or to ≤200 mg per 24 h; |
| | If the serum and urine M-protein are unmeasurable, a ≥50% decrease in |
| | the difference between involved and uninvolved FLC levels is required in |
| | place of the M-protein criteria; |
| | If serum and urine M-protein are unmeasurable, and serum-free light |
| | assay is also unmeasurable, ≥50% reduction in plasma cells is required in |
| | place of M-protein, provided baseline bone marrow plasma-cell |
| | percentage was ≥30%. In addition to these criteria, if present at baseline, |
| | a ≥50% reduction in the size (SPD)§§ of soft tissue plasmacytomas is |
| | also required |
| Minimal response | ≥25% but ≤49% reduction of serum M-protein and reduction in 24-h |
| | urine M-protein by 50-89%. In addition to the above listed criteria, if |
| | present at baseline, a ≥50% reduction in the size (SPD)§§ of soft tissue |
| | plasmacytomas is also required |
| Stable disease | Not recommended for use as an indicator of response; stability of disease |
| | is best described by providing the time-to-progression estimates. Not |
| | meeting criteria for complete response, very good partial response, partial |
| | response, minimal response, or progressive disease |
| Progressive disease ¶¶, \|\|\|\| | Any one or more of the following criteria: |
| | Increase of 25% from lowest confirmed response value in one or more of |
| | the following criteria: |
| | Serum M-protein (absolute increase must be ≥0.5 g/dL); |
| | Serum M-protein increase ≥1 g/dL, if the lowest M component was ≥5 |
| | g/dL; |
| | Urine M-protein (absolute increase must be ≥200 mg/24 h); |
| | In patients without measurable serum and urine M-protein levels, the |
| | difference between involved and uninvolved FLC levels (absolute |
| | increase must be ≥10 mg/dL); |
| | In patients without measurable serum and urine M-protein levels and |
| | without measurable involved FLC levels, bone marrow plasma-cell |
| | percentage irrespective of baseline status (absolute increase must |
| | be ≥10%); |
| | Appearance of a new lesion(s), ≥50% increase from nadir in SPD§§ |
| | of ≥1 lesion, or ≥50% increase in the longest diameter of a previous |
| | lesion ≥1 cm in short axis; |
| | ≥50% increase in circulating plasma cells (minimum of 200 cells per μL) |
| | if this is the only measure of disease |
| Clinical relapse | Clinical relapse requires one or more of the following criteria: |
| | Direct indicators of increasing disease and/or end organ dysfunction |
| | (CRAB features) related to the underlying clonal plasma-cell |
| | proliferative disorder. It is not used in calculation of time to progression |
| | or progression-free survival but is listed as something that can be |
| | reported optionally or for use in clinical practice; |
| | Development of new soft tissue plasmacytomas or bone lesions |
| | (osteoporotic fractures do not constitute progression); |
| | Definite increase in the size of existing plasmacytomas or bone lesions. A |
| | definite increase is defined as a 50% (and ≥1 cm) increase as measured |
| | serially by the SPD§§ of the measurable lesion; |
| | Hypercalcaemia (≥11 mg/dL); |
| | Decrease in haemoglobin of ≥2 g/dL not related to therapy or other non- |
| | myeloma-related conditions; |
| | Rise in serum creatinine by 2 mg/dL or more from the start of the therapy |
| | and attributable to myeloma; |
| | Hyperviscosity related to serum paraprotein |
| Relapse from complete | Any one or more of the following criteria: |
| response (to be used only | Reappearance of serum or urine M-protein by immunofixation or |
| if the end point is | electrophoresis; |
| disease-free survival) | Development of ≥5% plasma cells in the bone marrow; |
| | Appearance of any other sign of progression (i.e., new plasmacytoma, |
| | lytic bone lesion, or hypercalcaemia see above) |
| Relapse from MRD | Any one or more of the following criteria: |
| negative (to be used only | Loss of MRD negative state (evidence of clonal plasma cells on NGF or |
| if the end point is | NGS, or positive imaging study for recurrence of myeloma); |
| disease-free survival) | Reappearance of serum or urine M-protein by immunofixation or |
| | electrophoresis; |
| | Development of ≥5% clonal plasma cells in the bone marrow; |
| | Appearance of any other sign of progression (i.e., new plasmacytoma, |
| | lytic bone lesion, or hypercalcaemia) |

For MRD assessment, the first bone marrow aspirate should be sent to MRD (not for morphology) and this sample should be taken in one draw with a volume of minimally 2 mL (to obtain sufficient cells), but maximally 4-5 mL to avoid haemodilution. IMWG = International Myeloma Working Group. MRD = minimal residual disease. NGF = next-generation flow. NGS = next-generation sequencing. FLC = free light chain. M-protein = myeloma protein. SPD = sum of the products of the maximal perpendicular diameters of measured lesions. CRAB features = calcium elevation, renal failure, anaemia, lytic bone lesions. FCM = flow cytometry. SUVₘₐₓ = maximum standardised uptake value. MFC = multiparameter flow cytometry. ¹^{⁸}F-FDG PET = ¹^{⁸}F-fluorodeoxyglucose PET. ASCT = autologous stem cell transplantation.
*All response categories require two consecutive assessments made any time before starting any new therapy; for MRD there is no need for two consecutive assessments, but information on MRD after each treatment stage is recommended (eg, after induction, high-dose therapy/ASCT, consolidation, maintenance). MRD tests should be initiated only at the time of suspected complete response.
All categories of response and MRD require no known evidence of progressive or new bone lesions if radiographic studies were performed. However, radiographic studies are not required to satisfy these response requirements except for the requirement of FDG PET if imaging MRD- negative status is reported,†Sustained MRD negativity when reported should also annotate the method used (eg, sustained flow MRD-negative, sustained sequencing MRD-negative).† Bone marrow MFC should follow NGF guidelines (Paiva B, Gutierrez NC, Rosinol L, et al, Blood 2012; 119: 687-91). The reference NGF method is an eight-colour two-tube approach, which has been extensively validated. The two-tube approach improves reliability, consistency, and sensitivity because of the acquisition of a greater number of cells. The eight-colour technology is widely available globally and the NGF method has already been adopted in many flow laboratories worldwide. The complete eight-colour method is most efficient using a lyophilised mixture of antibodies which reduces errors, time, and costs. 5 million cells should be assessed. The FCM method employed should have a sensitivity of detection of at least 1 in 10⁵ plasma cells. §DNA sequencing assay on bone marrow aspirate should use a validated assay such as LymphoSIGHT (Sequenta).
¶Criteria used by Zamagni and colleagues (Zamagni E, Nanni C, Mancuso K, et al. Clin Cancer Res 2015; 21: 4384-90), and expert panel (IMPetUs; Italian Myeloma criteria for PET Use) (Usmani SZ, Mitchell A, Waheed S, et al. Blood 2013; 121: 1819-23; Nanni C, Zamagni E, Versari A, et al. Eur J Nucl Med Mol Imaging 2015; 43: 414-21.). Baseline positive lesions were identified by presence of focal areas of increased uptake within bones, with or without any underlying lesion identified by CT and present on at least two consecutive slices. Alternatively, an SUVₘₐₓ = 2.5 within osteolytic CT areas >1 cm in size, or SUVₘₐₓ = 1.5 within osteolytic CT areas ≤1 cm in size were considered positive. Imaging should be performed once MRD negativity is determined by MFC or NGS.
|||Derived from international uniform response criteria for multiple myeloma (Durie BG, Harousseau JL, Miguel JS, et al, Leukemia 2006; 20: 1467-73). Minor response definition and clarifications derived from Rajkumar and colleagues (Rajkumar SV, Harousseau JL, Durie B, et al, Blood 2011; 117: 4691-95). When the only method to measure disease is by serum FLC levels: complete response can be defined as a normal FLC ratio of 0.26 to 1.65 in addition to the complete response criteria listed previously. Very good partial response in such patients requires a ≥90% decrease in the difference between involved and uninvolved FLC levels. All response categories require two consecutive assessments made at any time before the institution of any new therapy; all categories also require no known evidence of progressive or new bone lesions or extramedullary plasmacytomas if radiographic studies were performed. Radiographic studies are not required to satisfy these response requirements. Bone marrow assessments do not need to be confirmed. Each category, except for stable disease, will be considered unconfirmed until the confirmatory test is performed. The date of the initial test is considered as the date of response for evaluation of time dependent outcomes such as duration of response.
**All recommendations regarding clinical uses relating to serum FLC levels or FLC ratio are based on results obtained with the validated Freelite test (Binding Site, Birmingham, UK).
††Presence/absence of clonal cells on immunohistochemistry is based upon the κ/λ/L ratio. An abnormal κ/λ ratio by immunohistochemistry requires a minimum of 100 plasma cells for analysis. An abnormal ratio reflecting presence of an abnormal clone is κ/λ of >4:1 or <1:2. ‡‡Special attention should be given to the emergence of a different monoclonal protein following treatment, especially in the setting of patients having achieved a conventional complete response, often related to oligoclonal reconstitution of the immune system. These bands typically disappear over time and in some studies have been associated with a better outcome. Also, appearance of monoclonal IgG κ in patients receiving monoclonal antibodies should be differentiated from the therapeutic antibody.
§§Plasmacytoma measurements should be taken from the CT portion of the PET/CT, or MRI scans, or dedicated CT scans where applicable. For patients with only skin involvement, skin lesions should be measured with a ruler. Measurement of tumour size will be determined by the SPD.
¶¶Positive immunofixation alone in a patient previously classified as achieving a complete response will not be considered progression. For purposes of calculating time to progression and progression-free survival, patients who have achieved a complete response and are MRD-negative should be evaluated using criteria listed for progressive disease. Criteria for relapse from a complete response or relapse from MRD should be used only when calculating disease-free survival.
||||In the case where a value is felt to be a spurious result per physician discretion (eg, a possible laboratory error), that value will not be considered when determining the lowest value.

Standard treatment for multiple myeloma and associated diseases includes chemotherapy, stem cell transplant (autologous or allogeneic), radiation therapy, and other drug therapies. Frequently used anti-myeloma drugs include alkylating agents (e.g., bendamustine, cyclophosphamide and melphalan), proteasome inhibitors (e.g., bortezomib), corticosteroids (e.g., dexamethasone and prednisone), and immunomodulators (e.g., thalidomide and lenalidomide or Revlimid®), or any combination thereof. Biphosphonate drugs are also frequently administered in combination with the standard anti-MM treamtents to prevent bone loss. Patients older than 65-70 years of age are unlikely candidates for stem cell transplant. In some cases, double-autologous stem cell transplants are options for patients less than 60 years of age with suboptimal response to the first transplant. The compositions and methods of the present invention may be administered in combination with any of the currently prescribed treatments for multiple myeloma.

The first phase of treatment for multiple myeloma is induction therapy. The goal of induction therapy is to reduce the number of plasma cells in the bone marrow and the molecules (e.g., proteins) produced by the plasma cells. Induction therapy usually comprises a combination of 2 or 3 of the following types of drugs: targeted therapy, chemotherapy, or corticosteroids.

Induction Therapy for Patients Who can have a Stem Cell Transplant

Patients for a stem cell transplant are usually 70 years of age or younger and in generally good health. Patients can have induction therapy followed by high-dose chemotherapy and a stem cell transplant. Induction therapy is usually given for several cycles and may include one or more of the following drugs: CyBorD regimen-cyclophosphamide (Cytoxan, Procytox), bortezomib (Velcade) and dexamethasone (Decadron, Dexasone); VRD regimen-bortezomib, lenalidomide (Revlimid) and dexamethasone; thalidomide (Thalomid) and dexamethasone; lenalidomide and low-dose dexamethasone; bortezomib and dexamethasone; VTD regimen-bortezomib, thalidomide and dexamethasone; bortezomib, cyclophosphamide and prednisone; bortezomib, doxorubicin (Adriamycin) and dexamethasone; dexamethasone; or liposomal doxorubicin (Caelyx, Doxil), vincristine (Oncovin) and dexamethasone

Induction Therapy for Patients Who Cannot have a Stem Cell Transplant

Patients who cannot have a stem cell transplant may have induction therapy using one or more of the following drugs: CyBorD regimen-cyclophosphamide, bortezomib and dexamethasone; lenalidomide (Revlimid) and low-dose dexamethasone; MPT regimen-melphalan, prednisone and thalidomide; VMP regimen-bortezomib, melphalan and prednisone; MPL regimen-melphalan, prednisone and lenalidomide; melphalan and prednisone; bortezomib and dexamethasone; dexamethasone; liposomal doxorubicin, vincristine and dexamethasone; thalidomide and dexamethasone; VAD regimen-vincristine, doxorubicin and dexamethasone; or VRD regimen-bortezomib, lenalidomide and dexamethasone.

Another example of a disease or disorder associated with BCMA is Hodgkin's lymphoma and non-Hodgkin's lymphoma (See Chiu et al., Blood. 2007, 109(2):729-39; He et al., J Immunol. 2004, 172(5):3268-79).

Hodgkin's lymphoma (HL), also known as Hodgkin's disease, is a cancer of the lymphatic system that originates from white blood cells, or lymphocytes. The abnormal cells that comprise the lymphoma are called Reed-Sternberg cells. In Hodgkin's lymphoma, the cancer spreads from one lymph node group to another. Hodgkin's lymphoma can be subclassified into four pathologic subtypes based upon Reed-Sternberg cell morphology and the cell composition around the Reed-Sternberg cells (as determined through lymph node biopsy): nodular sclerosing HL, mixed-cellularity subtype, lymphocyte-rich or lymphocytic predominance, lymphocyte depleted. Some Hodgkin's lymphoma can also be nodular lymphocyte predominant Hodgkin's lymphoma, or can be unspecified. Symptoms and signs of Hodgkin's lymphoma include painless swelling in the lymph nodes in the neck, armpits, or groin, fever, night sweats, weight loss, fatigue, itching, or abdominal pain.

Non-Hodgkin's lymphoma (NHL) comprises a diverse group of blood cancers that include any kind of lymphoma other than Hodgkin's lymphoma. Subtypes of non-Hodgkin's lymphoma are classified primarily by cell morphology, chromosomal aberrations, and surface markers. NHL subtypes (or NHL-associated cancers) include B cell lymphomas such as, but not limited to, Burkitt's lymphoma, B-cell chronic lymphocytic leukemia (B-CLL), B-cell prolymphocytic leukemia (B-PLL), chronic lymphocytic leukemia (CLL), diffuse large B-cell lymphoma (DLBCL) (e.g., intravascular large B-cell lymphoma and primary mediastinal B-cell lymphoma), follicular lymphoma (e.g., follicle center lymphoma, follicular small cleaved cell), hair cell leukemia, high grade B-cell lymphoma (Burkitt's like), lymphoplasmacytic lymphoma (Waldenstrom's macroglublinemia), mantle cell lymphoma, marginal zone B-cell lymphomas (e.g., extranodal marginal zone B-cell lymphoma or mucosa-associated lymphoid tissue (MALT) lymphoma, nodal marginal zone B-cell lymphoma, and splenic marginal zone B-cell lymphoma), plasmacytoma/myeloma, precursor B-lymphoblastic leukemia/lymphoma (PB-LBL/L), primary central nervous system (CNS) lymphoma, primary intraocular lymphoma, small lymphocytic lymphoma (SLL); and T cell lymphomas, such as, but not limited to, anaplastic large cell lymphoma (ALCL), adult T-cell lymphoma/leukemia (e.g., smoldering, chronic, acute and lymphomatous), angiocentric lymphoma, angioimmunoblastic T-cell lymphoma, cutaneous T-cell lymphomas (e.g., mycosis fungoides, Sezary syndrome, etc.), extranodal natural killer/T-cell lymphoma (nasal-type), enteropathy type intestinal T-cell lymphoma, large granular lymphocyte leukemia, precursor T-lymphoblastic lymphoma/leukemia (T-LBL/L), T-cell chronic lymphocytic leukemia/prolymphocytic leukemia (T-CLL/PLL), and unspecified peripheral T-cell lymphoma. Symptoms and signs of Hodgkin's lymphoma include painless swelling in the lymph nodes in the neck, armpits, or groin, fever, night sweats, weight loss, fatigue, itching, abdominal pain, coughing, or chest pain.

The staging is the same for both Hodgkin's and non-Hodgkin's lymphoma, and refers to the extent of spread of the cancer cells within the body. In stage I, the lymphoma cells are in one lymph node group. In stage II, lymphoma cells are present in at least two lymph node groups, but both groups are on the same side of the diaphragm, or in one part of a tissue or organ and the lymph nodes near that organ on the same side of the diaphragm. In stage III, lymphoma cells are in lymph nodes on both sides of the diaphragm, or in one part of a tissue or organ near these lymph node groups or in the spleen. In stage IV, lymphoma cells are found in several parts of at least one organ or tissue, or lymphoma cells are in an organ and in lymph nodes on the other side of the diaphragm. In addition to the Roman numeral staging designation, the stages of can also be described by letters A, B, E, and S, wherein A refers to patients without symptoms, B refers to patients with symptoms, E refers to patients in which lymphoma is found in tissues outside the lymph system, and S refers to patients in which lymphoma is found in the spleen.

Hodgkin's lymphoma is commonly treated with radiation therapy, chemotherapy, or hematopoietic stem cell transplantation. The most common therapy for non-Hodgkin's lymphoma is R-CHOP, which consists of four different chemotherapies (cyclophosphamide, doxorubicin, vincristine, and prenisolone) and rituximab (Rituxan®). Other therapies commonly used to treat NHL include other chemotherapeutic agents, radiation therapy, stem cell transplantation (autologous or allogeneic bone marrow transplantation), or biological therapy, such as immunotherapy. Other examples of biological therapeutic agents include, but are not limited to, rituximab (Rituxan®), tositumomab (Bexxar®), epratuzumab (LymphoCide®), and alemtuzumab (MabCampath®). The compositions and methods of the present invention may be administered in combination with any of the currently prescribed treatments for Hodgkin's lymphoma or non-Hodgkin's lymphoma.

BCMA expression has also been associated Waldenstrom's macroglobulinemia (WM), also known as lymphoplasmacytic lymphoma (LPL). (See Elsawa et al., Blood. 2006, 107(7):2882-8). Waldenstrom's macroglobulinemia was previously considered to be related to multiple myeloma, but has more recently been classified as a subtype of non-Hodgkin's lymphoma. WM is characterized by uncontrolled B-cell lymphocyte proliferation, resulting in anemia and production of excess amounts of paraprotein, or immunoglobulin M (IgM), which thickens the blood and results in hyperviscosity syndrome. Other symptoms or signs of WM include fever, night sweats, fatigue, anemia, weight loss, lymphadenopathy or splenomegaly, blurred vision, dizziness, nose bleeds, bleeding gums, unusual bruises, renal impairment or failure, amyloidosis, or peripheral neuropathy.

Standard treatment for WM consists of chemotherapy, specifically with rituximab (Rituxan®). Other chemotherapeutic drugs can be used in combination, such as chlorambucil (Leukeran®), cyclophosphamide (Neosar®), fludarabine (Fludara®), cladribine (Leustatin®), vincristine, and/or thalidomide. Corticosteriods, such as prednisone, can also be administered in combination with the chemotherapy. Plasmapheresis, or plasma exchange, is commonly used throughout treatment of the patient to alleviate some symptoms by removing the paraprotein from the blood. In some cases, stem cell transplantation is an option for some patients.

Another example of a disease or disorder associated with BCMA is brain cancer. Specifically, expression of BCMA has been associated with astrocytoma or glioblastoma (See Deshayes et al, Oncogene. 2004, 23(17):3005-12, Pelekanou et al., PLoS One. 2013, 8(12):e83250). Astrocytomas are tumors that arise from astrocytes, which are a type of glial cell in the brain. Glioblastoma (also known as glioblastoma multiforme or GBM) is the most malignant form of astrocytoma, and is considered the most advanced stage of brain cancer (stage IV). There are two variants of glioblastoma: giant cell glioblastoma and gliosarcoma. Other astrocytomas include juvenile pilocytic astrocytoma (JPA), fibrillary astrocytoma, pleomorphic xantroastrocytoma (PXA), desembryoplastic neuroepithelial tumor (DNET), and anaplastic astrocytoma (AA).

Symptoms or signs associated with glioblastoma or astrocytoma include increased pressure in the brain, headaches, seizures, memory loss, changes in behavior, loss in movement or sensation on one side of the body, language dysfunction, cognitive impairments, visual impairment, nausea, vomiting, and weakness in the arms or legs.

Surgical removal of the tumor (or resection) is the standard treatment for removal of as much of the glioma as possible without damaging or with minimal damage to the normal, surrounding brain. Radiation therapy and/or chemotherapy are often used after surgery to suppress and slow recurrent disease from any remaining cancer cells or satellite lesions. Radiation therapy includes whole brain radiotherapy (conventional external beam radiation), targeted three-dimensional conformal radiotherapy, and targeted radionuclides. Chemotherapeutic agents commonly used to treat glioblastoma include temozolomide, gefitinib or erlotinib, and cisplatin. Angiogenesis inhibitors, such as Bevacizumab (Avastin®), are also commonly used in combination with chemotherapy and/or radiotherapy.

Supportive treatment is also frequently used to relieve neurological symptoms and improve neurologic function, and is administered in combination any of the cancer therapies described herein. The primary supportive agents include anticonvulsants and corticosteroids. Thus, the compositions and methods of the present invention may be used in combination with any of the standard or supportive treatments to treat a glioblastoma or astrocytoma.

Non-cancer related diseases and disorders associated with BCMA expression can also be treated by the compositions and methods disclosed herein. Examples of non-cancer related diseases and disorders associated with BCMA expression include, but are not limited to: viral infections; e.g., HIV, fungal invections, e.g., C. neoformans; irritable bowel disease; ulcerative colitis, and disorders related to mucosal immunity.

The CAR-modified immune effector cells (e.g., T cells or NK cells) of the present invention may be administered either alone, or as a pharmaceutical composition in combination with diluents and/or with other components such as IL-2 or other cytokines or cell populations.

The present invention provides for compositions and methods for treating cancer. In one aspect, the cancer is a hematologic cancer including but is not limited to hematolical cancer is a leukemia or a lymphoma. In one aspect, the CAR-expressing cells (e.g., CART cells or CAR-expressing NK cells)of the invention may be used to treat cancers and malignancies such as, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with BCMA expression includes, but not limited to, e.g., atypical and/or non-classical cancers, malignancies, precancerous conditions or proliferative diseases expressing BCMA.

In embodiments, a composition described herein can be used to treat a disease including but not limited to a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

In embodiments, a composition described herein can be used to treat a disease including but not limited to a cancer, e.g., a cancer described herein, e.g., a prostate cancer (e.g., castrate-resistant or therapy-resistant prostate cancer, or metastatic prostate cancer), pancreatic cancer, or lung cancer.

The present invention also provides methods for inhibiting the proliferation or reducing a BCMA-expressing cell population, the methods comprising contacting a population of cells comprising a BMCA-expressing cell with an anti-BCMA CAR-expressing cell (e.g., BCMA CART cell or BCMA CAR-expressing NK cell)of the invention that binds to the BCMA-expressing cell. In a specific aspect, the present invention provides methods for inhibiting the proliferation or reducing the population of cancer cells expressing BCMA, the methods comprising contacting the BCMA-expressing cancer cell population with an anti-BCMA CAR-expressing cell (e.g., BCMA CART cell or BCMA CAR-expressing NK cell)of the invention that binds to the BCMA-expressing cell. In one aspect, the present invention provides methods for inhibiting the proliferation or reducing the population of cancer cells expressing BCMA, the methods comprising contacting the BMCA-expressing cancer cell population with an anti-BCMA CAR-expressing cell (e.g., BCMA CART cell or BCMA CAR-expressing NK cell)of the invention that binds to the BCMA-expressing cell. In certain aspects, the anti-BCMA CAR-expressing cell (e.g., BCMA CART cell or BCMA CAR-expressing NK cell)of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with BCMA-expressing cells relative to a negative control. In one aspect, the subject is a human.

The present invention also provides methods for preventing, treating and/or managing a disease associated with BCMA-expressing cells (e.g., a hematologic cancer or atypical cancer expessing BCMA), the methods comprising administering to a subject in need an anti-BCMA CAR-expressing cell (e.g., BCMA CART cell or BCMA CAR-expressing NK cell)of the invention that binds to the BCMA-expressing cell. In one aspect, the subject is a human. Non-limiting examples of disorders associated with BCMA-expressing cells include viral or fungal infections, and disorders related to mucosal immunity.

The present invention also provides methods for preventing, treating and/or managing a disease associated with BCMA-expressing cells, the methods comprising administering to a subject in need an anti-BCMA CAR-expressing cell (e.g., BCMA CART cell or BCMA CAR-expressing NK cell)of the invention that binds to the BCMA-expressing cell. In one aspect, the subject is a human.

The present invention provides methods for preventing relapse of cancer associated with BCMA-expressing cells, the methods comprising administering to a subject in need thereof an anti-BCMA CAR-expressing cell (e.g., BCMA CART cell or BCMA CAR-expressing NK cell)of the invention that binds to the BCMA-expressing cell. In one aspect, the methods comprise administering to the subject in need thereof an effective amount of an anti-BCMA CAR-expressing cell (e.g., BCMA CART cell or BCMA CAR-expressing NK cell)described herein that binds to the BCMA-expressing cell in combination with an effective amount of another therapy.

### Methods Using Biomarkers for Evaluating CAR-Effectiveness, Subject Suitability, or Sample Suitability

In another aspect, the invention features a method of evaluating or monitoring the effectiveness of a CAR-expressing cell therapy (e.g., a BCMA CAR therapy), in a subject (e.g., a subject having a cancer, e.g., a hematological cancer), or the suitability of a sample (e.g., an apheresis sample) for a CAR therapy (e.g., a BCMA CAR therapy). The method includes acquiring a value of effectiveness to the CAR therapy, subject suitability, or sample suitability, wherein said value is indicative of the effectiveness or suitability of the CAR-expressing cell therapy.

In some embodiments of any of the methods disclosed herein, the CAR-expressing cell therapy comprises a plurality (e.g., a population) of CAR-expressing immune effector cells, e.g., a plurality (e.g., a population) of T cells or NK cells, or a combination thereof. In one embodiment, the CAR-expressing cell therapy is a BCMACAR therapy.

In some embodiments of any of the methods disclosed herein, the subject is evaluated prior to receiving, during, or after receiving, the CAR-expressing cell therapy.

In some embodiments of any of the methods disclosed herein, a responder (e.g., a complete responder) has, or is identified as having, a greater level or activity of one, two, or more (all) of GZMK, PPF1BP2, or naïve T cells as compared to a non-responder.

In some embodiments of any of the methods disclosed herein, a non-responder has, or is identified as having, a greater level or activity of one, two, three, four, five, six, seven, or more (e.g., all) of IL22, IL-2RA, IL-21, IRF8, IL8, CCL17, CCL22, effector T cells, or regulatory T cells, as compared to a responder.

In an embodiment, a relapser is a patient having, or who is identified as having, an increased level of expression of one or more of (e.g., 2, 3, 4, or all of) the following genes, compared to non relapsers: MIR199A1, MIR1203, uc021ovp, ITM2C, and HLA-DQB1 and/or a decreased levels of expression of one or more of (e.g., 2, 3, 4, 5, 6, 7, 8, 9, 10, 11, or all of) the following genes, compared to non relapsers: PPIAL4D, TTTY10, TXLNG2P, MIR4650-1, KDMSD, USP9Y, PRKY, RPS4Y2, RPS4Y1, NCRNA00185, SULT1E1, and EIF1AY.

In some embodiments of any of the methods disclosed herein, a non-responder has, or is identified as having, a greater percentage of an immune cell exhaustion marker, e.g., one, two or more immune checkpoint inhibitors (e.g., PD-1, PD-L1, TIM-3 and/or LAG-3). In one embodiment, a non-responder has, or is identified as having, a greater percentage of PD-1, PD-L1, or LAG-3 expressing immune effector cells (e.g., CD4+ T cells and/or CD8+ T cells) (e.g., CAR-expressing CD4+ cells and/or CD8+ T cells) compared to the percentage of PD-1 or LAG-3 expressing immune effector cells from a responder.

In one embodiment, a non-responder has, or is identified as having, a greater percentage of immune cells having an exhausted phenotype, e.g., immune cells that co-express at least two exhaustion markers, e.g., co-expresses PD-1, PD-L1 and/or TIM-3. In other embodiments, a non-responder has, or is identified as having, a greater percentage of immune cells having an exhausted phenotype, e.g., immune cells that co-express at least two exhaustion markers, e.g., co-expresses PD-1 and LAG-3. In some embodiments of any of the methods disclosed herein, a non-responder has, or is identified as having, a greater percentage of PD-1/PD-L1+/LAG-3+ cells in the CAR-expressing cell population (e.g., a BCMACAR+ cell population) compared to a responder (e.g., a complete responder) to the CAR-expressing cell therapy.

In some embodiments of any of the methods disclosed herein, a partial responder has, or is identified as having, a higher percentages of PD-1/PD-L1+/LAG-3+ cells, than a responder, in the CAR-expressing cell population (e.g., a BCMACAR+ cell population).

In some embodiments of any of the methods disclosed herein, a non-responder has, or is identified as having, an exhausted phenotype of PD1/PD-L1+CAR+ and co-expression of LAG3 in the CAR-expressing cell population (e.g., a BCMACAR+ cell population).

In some embodiments of any of the methods disclosed herein, a non-responder has, or is identified as having, a greater percentage of PD-1/PD-L1+/TIM-3+ cells in the CAR-expressing cell population (e.g., a BCMACAR+ cell population) compared to the responder (e.g., a complete responder).

In some embodiments of any of the methods disclosed herein, a partial responders has, or is identified as having, a higher percentage of PD-1/PD-L1+/TIM-3+ cells, than responders, in the CAR-expressing cell population (e.g., a BCMACAR+ cell population).

In some embodiments of any of the methods disclosed herein, the presence of CD8+CD27+CD45RO− T cells in an apheresis sample is a positive predictor of the subject response to a CAR− expressing cell therapy (e.g., a BCMACAR therapy).

In some embodiments of any of the methods disclosed herein, a high percentage of PD1+CAR+ and LAG3+ or TIM3+ T cells in an apheresis sample is a poor prognostic predictor of the subject response to a CAR-expressing cell therapy (e.g., a BCMACAR therapy).

In some embodiments of any of the methods disclosed herein, the responder (e.g., the complete or partial responder) has one, two, three or more (or all) of the following profile:

(i) has a greater number of CD27+ immune effector cells compared to a reference value, e.g., a non-responder number of CD27+ immune effector cells;

(ii) (i) has a greater number of CD8+ T cells compared to a reference value, e.g., a non-responder number of CD8+ T cells;

(iii) has a lower number of immune cells expressing one or more checkpoint inhibitors, e.g., a checkpoint inhibitor chosen from PD-1, PD-L1, LAG-3, TIM-3, or KLRG-1, or a combination, compared to a reference value, e.g., a non-responder number of cells expressing one or more checkpoint inhibitors; or

(iv) has a greater number of one, two, three, four or more (all) of resting TEFF cells, resting TREG cells, naïve CD4 cells, unstimulated memory cells or early memory T cells, or a combination thereof, compared to a reference value, e.g., a non-responder number of resting TEFF cells, resting TREG cells, naïve CD4 cells, unstimulated memory cells or early memory T cells.

In some embodiments of any of the methods disclosed herein, the cytokine level or activity of (vi) is chosen from one, two, three, four, five, six, seven, eight, or more (or all) of cytokine CCL20/MIP3a, IL17A, IL6, GM-CSF, IFN-γ, IL10, IL13, IL2, IL21, IL4, IL5, IL9 or TNFα, or a combination thereof. The cytokine can be chosen from one, two, three, four or more (all) of IL-17a, CCL20, IL2, IL6, or TNFα. In one embodiment, an increased level or activity of a cytokine is chosen from one or both of IL-17a and CCL20, is indicative of increased responsiveness or decreased relapse.

In embodiments, the responder, a non-responder, a relapser or a non-relapser identified by the methods herein can be further evaluated according to clinical criteria. For example, a complete responder has, or is identified as, a subject having a disease, e.g., a cancer, who exhibits a complete response, e.g., a complete remission, to a treatment. A complete response may be identified, e.g., using the NCCN Guidelines®, or Cheson et al, J Clin Oncol 17:1244 (1999) and Cheson et al., “Revised Response Criteria for Malignant Lymphoma”, J Clin Oncol 25:579-586 (2007) (both of which are incorporated by reference herein in their entireties), as described herein. A partial responder has, or is identified as, a subject having a disease, e.g., a cancer, who exhibits a partial response, e.g., a partial remission, to a treatment. A partial response may be identified, e.g., using the NCCN Guidelines®, or Cheson criteria as described herein. A non-responder has, or is identified as, a subject having a disease, e.g., a cancer, who does not exhibit a response to a treatment, e.g., the patient has stable disease or progressive disease. A non-responder may be identified, e.g., using the NCCN Guidelines®, or Cheson criteria as described herein.

Alternatively, or in combination with the methods disclosed herein, responsive to said value, performing one, two, three four or more of:

administering e.g., to a responder or a non-relapser, a CAR-expressing cell therapy;

administered an altered dosing of a CAR-expressing cell therapy;

altering the schedule or time course of a CAR-expressing cell therapy;

administering, e.g., to a non-responder or a partial responder, an additional agent in combination with a CAR-expressing cell therapy, e.g., a checkpoint inhibitor, e.g., a checkpoint inhibitor described herein;

administering to a non-responder or partial responder a therapy that increases the number of younger T cells in the subject prior to treatment with a CAR-expressing cell therapy;

modifying a manufacturing process of a CAR-expressing cell therapy, e.g., enriching for younger T cells prior to introducing a nucleic acid encoding a CAR, or increasing the transduction efficiency, e.g., for a subject identified as a non-responder or a partial responder;

administering an alternative therapy, e.g., for a non-responder or partial responder or relapser; or

if the subject is, or is identified as, a non-responder or a relapser, decreasing the TREG cell population and/or T_{REG} gene signature, e.g., by one or more of CD25 depletion, administration of cyclophosphamide, anti-GITR antibody, or a combination thereof.

In certain embodiments, the subject is pre-treated with an anti-GITR antibody. In certain embodiment, the subject is treated with an anti-GITR antibody prior to infusion or re-infusion.

### Combination Therapies

A CAR-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

The CAR therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder.

When administered in combination, the CAR therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

### Thalidomide Class of Compounds

In some embodiments, the BCMA CAR-expressing cell therapy is administered in combination with a member of the thalidomide class of compounds. In some embodiments, members of the thalidomide class of compounds include, but are not limited to, lenalidomide (CC-5013), pomalidomide (CC-4047 or ACTIMID), thalidomide, or salts or derivatives thereof. In some embodiments, the compound can be a mixture of one, two, three, or more members of the thalidomide class of compounds. Thalidomide analogs and immunomodulatory properties of thalidomide analogs are described in Bodera and Stankiewicz, Recent Pat Endocr Metab Immune Drug Discov. 2011 September; 5(3):192-6, which is hereby incorporated by reference in its entirety. The structural complex of thalidomide analogs and the E3 ubiquitin is described in Gandhi et al., Br J Haematol. 2014 March; 164(6):811-21, which is hereby incorporated by reference in its entirety. The modulation of the E3 ubiquitin ligase by thalidomide analogs is described in Fischer et al., Nature. 2014 Aug. 7; 512(7512):49-53, which is hereby incorporated by reference in its entirety.

In some embodiments, the compound comprises a compound of Formula (I):

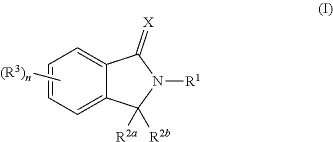

or a pharmaceutically acceptable salt, ester, hydrate, solvate, or tautomer thereof, wherein:

X is O or S;

R¹ is C₁-C₆ alkyl, C₂-C₆ alkenyl, C₂-C₆ alkynyl, C₁-C₆ heteroalkyl, carbocyclyl, heterocyclyl, aryl, or heteroaryl, each of which is optionally substituted by one or more R⁴;

each of R² and R^{2b} is independently hydrogen or C₁-C₆ alkyl; or R² and R^{2b} together with the carbon atom to which they are attached form a carbonyl group or a thiocarbonyl group;

each of R³ is independently C₁-C₆ alkyl, C₂-C₆ alkenyl, C₂-C₆ alkynyl, C₁-C₆ heteroalkyl, halo, cyano, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)ₓN(R^{C})(R^{D}), or —N(R^{C})S(O)ₓR^{E}, wherein each alkyl, alkenyl, alkynyl, and heteroalkyl is independently and optionally substituted with one or more R⁶;

each R⁴ is independently C₁-C₆ alkyl, C₂-C₆ alkenyl, C₂-C₆ alkynyl, C₁-C₆ heteroalkyl, halo, cyano, oxo, —C(O)R^{A}, —C(O)OR^{B}, —OR^{B}, —N(R^{C})(R^{D}), —C(O)N(R^{C})(R^{D}), —N(R^{C})C(O)R^{A}, —S(o)ₓR^{E}, —S(O)ₓN(R^{C})(R^{D}), —N(R^{C})S(O)ₓ(R^{E}, carbocyclyl, heterocyclyl, aryl, or heteroaryl, wherein each alkyl, alkenyl, alkynyl, heteroalkyl, carbocyclyl, heterocyclyl, aryl, and heteroaryl is independently and optionally substituted with one or more IV;

each of R^{A}, R^{B}, R^{C}, R^{D}, and R^{E} is independently hydrogen or C₁-C₆ alkyl;

each R⁶ is independently C₁-C₆ alkyl, oxo, cyano, —OR^{B}, —N(R^{C})(R^{D}), —C(O)N(R^{C})(R^{D}), —N(R^{C})C(O)R^{A}, aryl, or heteroaryl, wherein each aryl and heteroaryl is independently and optionally substituted with one or more R⁸;

each R⁷ is independently halo, oxo, cyano, —OR^{B}, —N(R^{C})(R^{D}), —C(O)N(R^{C})(R^{D}), or —N(R^{C})C(O)R^{A};

each R⁸ is independently C₁-C₆ alkyl, cyano, —OR^{B}, —N(R^{C})(R^{D}), —C(O)N(R^{C})(R^{D}), or —N(R^{C})C(O)R^{A};

n is 0, 1, 2, 3 or 4; and

x is 0, 1, or 2.

In some embodiments, X is O.

In some embodiments, R¹ is heterocyclyl. In some embodiments, R¹ is a 6-membered heterocyclyl or a 5-membered heterocyclyl. In some embodiments, R¹ is a nitrogen-containing heterocyclyl. In some embodiments, R¹ is piperidinyl (e.g., piperidine-2,6-dionyl).

In some embodiments, each of R^{2a} and R^{2b} is independently hydrogen. In some embodiments, R^{2a} and R^{2b} together with the carbon to which they are attached form a carbonyl group.

In some embodiments, R³ is C₁-C₆ heteroalkyl, —N(R^{C})(R^{D}) or —N(R^{C})C(O)R^{A}. In some embodiments, R³ is C₁-C₆ heteroalkyl (e.g., CH₂NHC(O)CH₂-phenyl-t-butyl), —N(R^{C})(R^{D}) (e.g., NH₂), or —N(R^{C})C(O)R^{A} (e.g., NHC(O)CH₃).

In an embodiment, X is O. In an embodiment, R¹ is heterocyclyl (e.g., piperidine-2,6-dionyl). In an embodiment, each of R^{2a} and R^{2b} is independently hydrogen. In an embodiment, n is 1. In an embodiment, R³ is —N(R^{C})(R^{D}) (e.g., —NH₂). In an embodiment, the compound comprises lenalidomide, e.g., 3-(4-amino-1-oxoisoindolin-2-yl)piperidine-2,6-dione, or a pharmaceutically acceptable salt thereof. In an embodiment, the compound is lenalidomide, e.g., according to the following formula:

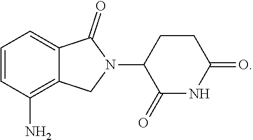 [SMILES: Nc1cccc2c1CN(C1CCC(=O)NC1=O)C2=O]

In an embodiment, X is O. In an embodiment, R¹ is heterocyclyl (e.g., piperidinyl-2,6-dionyl). In some embodiments, R^{2a} and R^{2b} together with the carbon to which they are attached form a carbonyl group. In an embodiment, n is 1. In an embodiment, R³ is —N(R^{C})(R^{D}) (e.g., —NH₂). In an embodiment, the compound comprises pomalidomide, e.g., 4-amino-2-(2,6-dioxopiperidin-3-yl)isoindoline-1,3-dione, or a pharmaceutically acceptable salt thereof. In an embodiment, the compound is pomalidomide, e.g., according to the following formula:

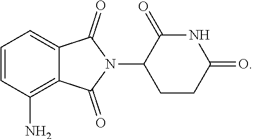 [SMILES: Nc1cccc2c1C(=O)N(C1CCC(=O)NC1=O)C2=O]

In an embodiment, X is O. In an embodiment, R¹ is heterocyclyl (e.g., piperidinyl-2,6-dionyl). In an embodiment, R^{2a} and R^{2b} together with the carbon to which they are attached form a carbonyl group. In an embodiment, n is 0. In an embodiment, the compound comprises thalidomide, e.g., 2-(2,6-dioxopiperidin-3-yl)isoindoline-1,3-dione, or a pharmaceutically acceptable salt thereof. In an embodiment, the product is thalidomide, e.g., according to the following formula:

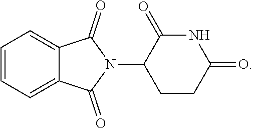 [SMILES: O=C1CCC(N2C(=O)c3ccccc3C2=O)C(=O)N1]

In an embodiment, X is O. In an embodiment, R¹ is heterocyclyl (e.g., piperidine-2,6-dionyl). In an embodiment, each of R^{2a} and R^{2b} is independently hydrogen. In an embodiment, n is 1. In an embodiment, R³ is C₁-C₆ heteroalkyl (e.g., CH₂NHC(O)CH₂-phenyl-t-butyl) In an embodiment, the compound comprises 2-(4-(tert-butyl)phenyl)-N-((2-(2,6-dioxopiperidin-3-yl)-1-oxoisoindolin-5-yl)methyl)acetamide, or a pharmaceutically acceptable salt thereof. In an embodiment, the compound has the structure as shown in the following formula:

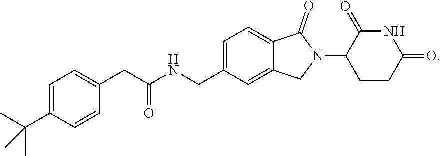 [SMILES: CC(C)(C)c1ccc(CC(=O)NCc2ccc3c(c2)CN(C2CCC(=O)NC2=O)C3=O)cc1]

In some embodiments, the compound is a compound of Formula (I-a):

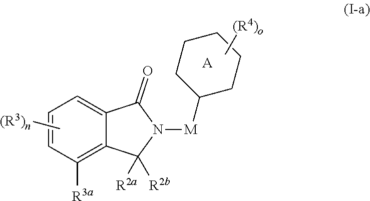

or a pharmaceutically acceptable salt, ester, hydrate, or tautomer thereof, wherein:

Ring A is carbocyclyl, heterocyclyl, aryl, or heteroaryl, each of which optionally substituted with one or more R⁴;

M is absent, C₁-C₆ alkyl, C₂-C₆ alkenyl, C₂-C₆ alkynyl, or C₁-C₆ heteroalkyl, wherein each alkyl, alkenyl, alkynyl, and heteroalkyl is optionally substituted with one or more R⁴;

each of R^{2a} and R^{2b} is independently hydrogen or C₁-C₆ alkyl; or R^{2a} and R^{2b} together with the carbon atom to which they are attached to form a carbonyl group or thiocarbonyl group;

R^{3a} is hydrogen, C₁-C₆ alkyl, C₂-C₆ alkenyl, C₂-C₆ alkynyl, C₁-C₆ heteroalkyl, halo, cyano, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)—N(R^{C})(R^{D}), or —N(R^{C})S(O)ₓR^{E}, wherein each alkyl, alkenyl, alkynyl, and heteroalkyl is optionally substituted with one or more R⁶;

each of R³ is independently C₁-C₆ alkyl, C₂-C₆ alkenyl, C₂-C₆ alkynyl, C₁-C₆ heteroalkyl, halo, cyano, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)ₓN(R^{C})(R^{D}), or —N(R^{C})S(O)ₓR^{E}, wherein each alkyl, alkenyl, alkynyl, and heteroalkyl is independently and optionally substituted with one or more R⁶;

each R⁴ is independently C₁-C₆ alkyl, C₂-C₆ alkenyl, C₂-C₆ alkynyl, C₁-C₆ heteroalkyl, halo, cyano, oxo, —C(O)R^{A}, —C(O)OR^{B}, —OR^{B}, —N(R^{C})(R^{D}), —C(O)N(R^{C})(R^{D}), —N(R^{C})C(O)R^{A}, S(O)ₓR^{E}, —S(O)ₓN(R^{C})(R^{D}), —N(R^{C})S(O)ₓR^{E}, carbocyclyl, heterocyclyl, aryl, or heteroaryl, wherein each alkyl, alkenyl, alkynyl, carbocyclyl, heterocyclyl, aryl, or heteroaryl is independently and optionally substituted with one or more IV;

each of R^{A}, R^{B}, R^{C}, R^{D}, and R^{E} is independently hydrogen or C₁-C₆ alkyl;

each R⁶ is independently C₁-C₆ alkyl, oxo, cyano, —OR^{B}, —N(R^{C})(R^{D}), —C(O)N(R^{C})(R^{D}), —N(R^{C})C(O)R^{A}, aryl, or heteroaryl, wherein each aryl or heteroaryl is independently and optionally substituted with one or more R⁸;

each R⁷ is independently halo, oxo, cyano, —OR^{B}, —N(R^{C})(R^{D}), —C(O)N(R^{C})(R^{D}), or —N(R^{C})C(O)R^{A};

each R⁸ is independently C₁-C₆ alkyl, cyano, —OR^{B}, —N(R^{C})(R^{D}), —C(O)N(R^{C})(R^{D}), or —N(R^{C})C(O)R^{A};

n is 0, 1, 2, or 3;

o is 0, 1, 2, 3, 4, or 5; and

x is 0, 1, or 2.

In some embodiments, X is O.

In some embodiments, M is absent.

In some embodiments, Ring A is heterocyclyl. In some embodiments, Ring A is heterocyclyl, e.g., a 6-membered heterocyclyl or a 5-membered heterocyclyl. In some embodiments, Ring A is a nitrogen-containing heterocyclyl. In some embodiments, Ring A is piperidinyl (e.g., piperidine-2,6-dionyl).

In some embodiments, M is absent and Ring A is heterocyclyl (e.g., piperidinyl, e.g., piperidine-2,6-dionyl).

In some embodiments, each of R^{2a} and R^{2b} is independently hydrogen. In some embodiments, R^{2a} and R^{2b} together with the carbon to which they are attached form a carbonyl group.

In some embodiments, R^{1a} is hydrogen, —N(R^{C})(R^{D}) or —N(R^{C})C(O)R^{A}. In some embodiments, R^{1a} is hydrogen. In some embodiments, R^{1a} is —N(R^{C})(R^{D}) (e.g., —NH₂). In some embodiments, R^{1a} is —N(R^{C})C(O)R^{A} (e.g, NHC(O)CH₃).

In some embodiments, R³ is C₁-C₆ heteroalkyl (e.g., CH₂NHC(O)CH₂-phenyl-t-butyl). In some embodiments, n is 0 or 1. In some embodiments, n is 0. In some embodiments, n is 1.

The compound may comprise one or more chiral centers or exist as one or more stereoisomers. In some embodiments, the compound comprises a single chiral center and is a mixture of stereoisomers, e.g., an R stereoisomer and an S stereoisomer. In some embodiments, the mixture comprises a ratio of R stereoisomers to S stereoisomers, for example, about a 1:1 ratio of R stereoisomers to S stereoisomers (i.e., a racemic mixture). In some embodiments, the mixture comprises a ratio of R stereoisomers to S stereoisomers of about 51:49, about 52: 48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of S stereoisomers to R stereoisomers of about 51:49, about 52: 48, about 53:47, about 54:46, about 55:45, about 60:40, about 65:35, about 70:30, about 75:25, about 80:20, about 85:15, about 90:10, about 95:5, or about 99:1. In some embodiments, the compound is a single stereoisomer of Formula (I) or Formula (I-a), e.g., a single R stereoisomer or a single S stereoisomer.

### Kinase Inhibitors

In some embodiments, the BCMA CAR-expressing cell therapy is administered in combination with a kinase inhibitor. In one embodiment, the kinase inhibitor is a CDK4 inhibitor, e.g., a CDK4 inhibitor described herein, e.g., a CDK4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is a BTK inhibitor, e.g., a BTK inhibitor described herein, such as, e.g., ibrutinib. In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo [3,4-d] pyrimidine. The MNK inhibitor can be, e.g., a MNK1a, MNK1b, MNK2a and/or MNK2b inhibitor. In one embodiment, the kinase inhibitor is a dual PI3K/mTOR inhibitor described herein, such as, e.g., PF-04695102. In one embodiment, the kinase inhibitor is a DGK inhibitor, e.g., a DGK inhibitor described herein, such as, e.g., DGKinh1 (D5919) or DGKinh2 (D5794).

In one embodiment, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In a preferred embodiment, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In one embodiment, the kinase inhibitor is a BTK inhibitor, e.g., ibrutinib (PCI-32765). In embodiments, a CAR-expressing cell described herein is administered to a subject in combination with a BTK inhibitor (e.g., ibrutinib). In embodiments, a CAR-expressing cell described herein is administered to a subject in combination with ibrutinib (also called PCI-32765). The structure of ibrutinib (1-[(3R)-3-[4-Amino-3-(4-phenoxyphenyl)-1H-pyrazolo[3,4-d]pyrimidin-1-yl]piperidin-1-yl]prop-2-en-1-one) is shown below.

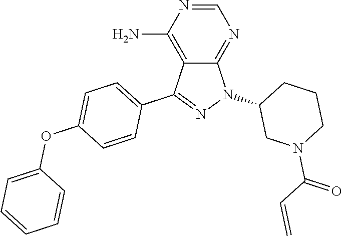 [SMILES: C=CC(=O)N1CCC[C@@H](n2nc(-c3ccc(Oc4ccccc4)cc3)c3c(N)ncnc32)C1]

In embodiments, the subject has CLL, mantle cell lymphoma (MCL), or small lymphocytic lymphoma (SLL). For example, the subject has a deletion in the short arm of chromosome 17 (del(17p), e.g., in a leukemic cell). In other examples, the subject does not have a del(17p). In embodiments, the subject has relapsed CLL or SLL, e.g., the subject has previously been administered a cancer therapy (e.g., previously been administered one, two, three, or four prior cancer therapies). In embodiments, the subject has refractory CLL or SLL. In other embodiments, the subject has follicular lymphoma, e.g., relapse or refractory follicular lymphoma. In some embodiments, ibrutinib is administered at a dosage of about 300-600 mg/day (e.g., about 300-350, 350-400, 400-450, 450-500, 500-550, or 550-600 mg/day, e.g., about 420 mg/day or about 560 mg/day), e.g., orally. In embodiments, the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of ibrutinib are administered. In some embodiments, ibrutinib is administered in combination with rituximab. See, e.g., Burger et al. (2013) Ibrutinib In Combination With Rituximab (iR) Is Well Tolerated and Induces a High Rate Of Durable Remissions In Patients With High-Risk Chronic Lymphocytic Leukemia (CLL): New, Updated Results Of a Phase II Trial In 40 Patients, Abstract 675 presented at 55^{th} ASH Annual Meeting and Exposition, New Orleans, La. 7-10 December Without being bound by theory, it is thought that the addition of ibrutinib enhances the T cell proliferative response and may shift T cells from a T-helper-2 (Th2) to T-helper-1 (Th1) phenotype. Th1 and Th2 are phenotypes of helper T cells, with Th1 versus Th2 directing different immune response pathways. A Th1 phenotype is associated with proinflammatory responses, e.g., for killing cells, such as intracellular pathogens/viruses or cancerous cells, or perpetuating autoimmune responses. A Th2 phenotype is associated with eosinophil accumulation and anti-inflammatory responses.

### EGFR Inhibitors

In some embodiments, the BCMA CAR-expressing cell therapy is administered in combination with an inhibitor of Epidermal Growth Factor Receptor (EGFR).

In some embodiments, the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide (Compound A40) or a compound disclosed in PCT Publication No. WO 2013/184757.

In some embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A40), or a compound disclosed in PCT Publication No. WO 2013/184757, is a covalent, irreversible tyrosine kinase inhibitor. In certain embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A40), or a compound disclosed in PCT Publication No. WO 2013/184757 inhibits activating EGFR mutations (L858R, ex19del). In other embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A40), or a compound disclosed in PCT Publication No. WO 2013/184757 does not inhibit, or does not substantially inhibit, wild-type (wt) EGFR. Compound A40 has shown efficacy in EGFR mutant NSCLC patients. In some embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A40), or a compound disclosed in PCT Publication No. WO 2013/184757 also inhibits one or more kinases in the TEC family of kinases. The Tec family kinases include, e.g., ITK, BMX, TEC, RLK, and BTK, and are central in the propogation of T-cell receptor and chemokine receptor signaling (Schwartzberg et al. (2005) Nat. Rev. Immunol. p. 284-95). For example, Compound A40 can inhibit ITK with a biochemical IC50 of 1.3 nM. ITK is a critical enzyme for the survival of Th2 cells and its inhibition results in a shift in the balance between Th2 and Th1 cells. In some embodiments, the EGFR inhibitor is chosen from one of more of erlotinib, gefitinib, cetuximab, panitumumab, necitumumab, PF-00299804, nimotuzumab, or R05083945.

### Adenosine A2A Receptor Inhibitors

In some embodiments, the BCMA CAR-expressing cell therapy is administered in combination with an adenosine A2a receptor (A2aR) antagonist (e.g., an inhibitor of A2aR pathway, e.g., an adenosine inhibitor, e.g., an inhibitor of A2aR or CD-73). In some embodiments, the A2aR antagonist is chosen from PBF509 (Palobiofarma/Novartis), CPI444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), or Preladenant/SCH 420814 (Merck/Schering).

In some embodiments, the A2aR antagonist comprises PBF509 or a compound disclosed in U.S. Pat. No. 8,796,284 or in International Application Publication No. WO 2017/025918, herein incorporated by reference in their entirety.

In some embodiments, the A2aR antagonist comprises a compound of formula (I):

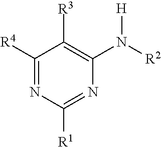

wherein

R¹represents a five-membered heteroaryl ring selected from the group consisting of a pyrazole, a thiazole, and a triazole ring optionally substituted by one or two halogen atoms or by one or two methyl groups;

R² represents a hydrogen atom;

R³ represents bromine or chlorine atom;

R⁴ represents independently:

a) a five-membered heteroaryl group optionally substituted by one or more halogen atoms or by one or more groups selected from the group consisting of alkyl, cycloalkyl, alkoxy, alkylthio, amino, mono- or dialkylamino

b) a group —N(R⁵)(R⁶) in which R⁵ and R⁶ represent independently:

a hydrogen atom;

an alkyl or cycloalkyl group of 3 to 6 carbon atoms, linear or branched, optionally substituted by one or more halogen atoms or by one or more groups selected from the group consisting of cycloalkyl (3-8 carbon atoms), hydroxy, alkoxy, amino, mono- and dialkylamino (1-8 carbon atoms);

or R⁵ and R⁶ form together with the nitrogen atom to that they are attached a saturated heterocyclic group of 4 to 6 members in which further heteroatom may be inserted, which is optionally substituted by one or more halogen atoms or by one or more alkyl groups (1-8 carbon atoms), hydroxy, lower alkoxy, amino, mono- or dialkylamino, or

c) a group —OR′ or —SR′, where R⁷ represents independently:

an alkyl (1-8 carbon atoms) or cycloalkyl (3-8 carbon atoms) group, linear or branched, optionally substituted by one or more halogen atoms or by one or more groups selected from the group consisting of alkyl (1-8 carbon atoms), alkoxy (1-8 carbon atoms), amino, mono- or dialkylamino (1-8 carbon atoms); or

a Phenyl ring optionally substituted with one or more halogen atoms.

In certain embodiments, the A2aR antagonist comprises 5-bromo-2,6-di-(1H-pyrazol-1-yl)pyrimidin-4-amine

In certain embodiments, the A2AR antagonist comprises CPI444/V81444. CPI-444 and other A2aR antagonists are disclosed in International Application Publication No. WO 2009/156737, herein incorporated by reference in its entirety. In certain embodiments, the A2aR antagonist is (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine. In certain embodiments, the A2aR antagonist is (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof. In certain embodiments, the A2aR antagonist is 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine

In certain embodiments, the A2aR antagonist is AZD4635/HTL-1071. A2aR antagonists are disclosed in International Application Publication No. WO 2011/095625, herein incorporated by reference in its entirety. In certain embodiments, the A2aR antagonist is 6-(2-chloro-6-methylpyridin-4-yl)-5-(4-fluorophenyl)-1,2,4-triazin-3-amine.

In certain embodiments, the A2aR antagonist is ST-4206 (Leadiant Biosciences). In certain embodiments, the A2aR antagonist is an A2aR antagonist described in U.S. Pat. No. 9,133,197, herein incorporated by reference in its entirety.

In certain embodiments, the A2AR antagonist is an A2aR antagonist described in U.S. Pat. Nos. 8,114,845 and 9,029,393, U.S. Application Publication Nos. 2017/0015758 and 2016/0129108, herein incorporated by reference in their entirety.

In some embodiments, the A2aR antagonist is istradefylline (CAS Registry Number: 155270-99-8). Istradefylline is also known as KW-6002 or 8-[(E)-2-(3,4-dimethoxyphenyl)vinyl]-1,3-diethyl-7-methyl-3,7-dihydro-1H-purine-2,6-dione. Istradefylline is disclosed, e.g., in LeWitt et al. (2008) Annals of Neurology 63 (3): 295-302).

In some embodiments, the A2aR antagonist is tozadenant (Biotie). Tozadenant is also known as SYN115 or 4-hydroxy-N-(4-methoxy-7-morpholin-4-yl-1,3-benzothiazol-2-yl)-4-methylpiperidine-1-carboxamide. Tozadenant blocks the effect of endogenous adenosine at the A2a receptors, resulting in the potentiation of the effect of dopamine at the D2 receptor and inhibition of the effect of glutamate at the mGluR5 receptor. In some embodiments, the A2aR antagonist is preladenant (CAS Registry Number: 377727-87-2). Preladenant is also known as SCH 420814 or 2-(2-Furanyl)-7-[2-[4-[4-(2-methoxyethoxy)phenyl]-1-piperazinyl]ethyl]7H-pyrazolo[4,3-e][1,2,4]triazolo[1,5-c]pyrimidine-5-amine Preladenant was developed as a drug that acted as a potent and selective antagonist at the adenosine A2A receptor.

In some embodiments, the A2aR antagonist is vipadenan. Vipadenan is also known as BIIB014, V2006, or 3-[(4-amino-3-methylphenyl)methyl]-7-(furan-2-yl)triazolo[4,5-d]pyrimidin-5-amine

Other exemplary A2aR antagonists include, e.g., ATL-444, MSX-3, SCH-58261, SCH-412,348, SCH-442,416, VER-6623, VER-6947, VER-7835, CGS-15943, or ZM-241,385.

### IDO/TDO Inhibitors

In some embodiments, the BCMA CAR-expressing cell therapy is administered in combination with an inhibitor of indoleamine 2,3-dioxygenase (IDO) and/or tryptophan 2,3-dioxygenase (TDO). In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod (NLG8189), (1-methyl-D-tryptophan), α-cyclohexyl-5H-Imidazo[5,1-a]isoindole-5-ethanol (also known as NLG919), indoximod, BMS-986205 (formerly F001287).

In some embodiments, the IDO/TDO inhibitor is indoximod (New Link Genetics). Indoximod, the D isomer of 1-methyl-tryptophan, is an orally administered small-molecule indoleamine 2,3-dioxygenase (IDO) pathway inhibitor that disrupts the mechanisms by which tumors evade immune-mediated destruction.

In some embodiments, the IDO/TDO inhibitor is NLG919 (New Link Genetics). NLG919 is a potent IDO (indoleamine-(2,3)-dioxygenase) pathway inhibitor with Ki/EC50 of 7 nM/75 nM in cell-free assays.

In some embodiments, the IDO/TDO inhibitor is epacadostat (CAS Registry Number: 1204669-58-8). Epacadostat is also known as INCB24360 or INCB024360 (Incyte). Epacadostat is a potent and selective indoleamine 2,3-dioxygenase (IDO1) inhibitor with IC50 of 10 nM, highly selective over other related enzymes such as IDO2 or tryptophan 2,3-dioxygenase (TDO).

In some embodiments, the IDO/TDO inhibitor is F001287 (Flexus/BMS). F001287 is a small molecule inhibitor of indoleamine 2,3-dioxygenase 1 (IDO1).

### CD19 CAR

In some embodiments, the BCMA CAR-expressing cell therapy is administered in combination with a CD19 CAR-expressing cell therapy.

In one embodiment, the antigen binding domain of the CD19 CAR has the same or a similar binding specificity as the FMC63 scFv fragment described in Nicholson et al. Mol. Immun 34 (16-17): 1157-1165 (1997). In one embodiment, the antigen binding domain of the CD19 CAR includes the scFv fragment described in Nicholson et al. Mol. Immun. 34 (16-17): 1157-1165 (1997).

In some embodiments, the CD19 CAR includes an antigen binding domain (e.g., a humanized antigen binding domain) according to Table 3 of WO2014/153270, incorporated herein by reference.

WO2014/153270 also describes methods of assaying the binding and efficacy of various CAR constructs.

In one aspect, the parental murine scFv sequence is the CAR19 construct provided in PCT publication WO2012/079000 (incorporated herein by reference). In one embodiment, the anti-CD19 binding domain is a scFv described in WO2012/079000.

In one embodiment, the CAR molecule comprises the fusion polypeptide sequence provided as SEQ ID NO: 12 in PCT publication WO2012/079000, which provides an scFv fragment of murine origin that specifically binds to human CD19.

In one embodiment, the CD19 CAR comprises an amino acid sequence provided as SEQ ID NO: 12 in PCT publication WO2012/079000. In embodiment, the amino acid sequence is

(SEQ ID NO: 2029)
(MALPVTALLLPLALLLHAARP)diqmtqttsslsaslgdrvtiscrasq
diskylnwyqqkpdgtvklliyhtsrlhsgvpsrfsgsgsgtdysitisn
leqediatyfcqqgntlpytfgggtkleitggggsggggsggggsevklq
esgpglvapsqslsvtctvsgvslpdygvswirqpprkglewlgviwgse
ttyynsalksrltiikdnsksqvflkmnslqtddtaiyycakhyyyggsy
amdywgqgtsvtvsstapaprpptpaptiasqplslrpeacrpaaggavh
trgldfacdiyiwaplagtcgvlllslvitlyckrgrkkllyifkqpfmr
pvqttqeedgcscrfpeeeeggcelrvkfsrsadapaykqgqnqlyneln
lgrreeydvldlargrdpemggkprrknpqeglynelqkdkmaeayseig
mkgeragkghdglyqglstatkdtydalhmqalppr,

or a sequence substantially homologous thereto. The optional sequence of the signal peptide is shown in capital letters and parenthesis.

In one embodiment, the amino acid sequence is:

(SEQ ID NO: 2030)
Diqmtqttsslsaslgdrvtiscrasqdiskylnwyqqkpdgtvklliyh
tsrlhsgvpsrfsgsgsgtdysltisnleqediatyfcqqgntlpytfgg
gtkleitggggsggggsggggsevklqesgpglvapsqslsvtctvsgvs
lpdygvswirqpprkglewlgviwgsettyynsalksrltiikdnsksqv
flkmnslqtddtaiyycakhyyyggsyamdywgqgtsvtvsstapaprpp
tpaptiasqplslrpeacrpaaggavhtrgldfacdiyiwaplagtcgvl
llslvitlyckrgrkkllyifkqpfmrpvqttqeedgcscrfpeeeeggc
elrvkfsrsadapaykqgqnqlynelnlgrreeydvldkagrdpemggkp
rrknpqeglynelqkdkmaeayseigmkgeragkghdglyqglstatkdt
ydalhmqalppr,

or a sequence substantially homologous thereto.

In one embodiment, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (e.g., a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, CD19 CAR constructs are described in PCT publication WO 2012/079000, incorporated herein by reference, and the amino acid sequence of the murine CD19 CAR and scFv constructs are shown in Table 8 below, or a sequence substantially identical to any of the aforesaid sequences (e.g., at least 85%, 90%, 95% or more identical to any of the sequences described herein).

TABLE 8
CD19 CAR Constructs

| SEQ ID NO | Region |
|---|---|
| CTL019 | |
| 1300 | CTL019 Full amino acid sequence |
| 1301 | CTL019 Full nucleotide sequence |
| 1302 | CTL019 scFv domain |
| mCAR1 | |
| 1303 | mCAR1 scFv |
| 1304 | mCAR1 Full amino acid sequence |
| mCAR2 | |
| 1305 | mCAR2 scFv |
| 1306 | mCAR2 amino acid sequence |
| 1307 | mCAR2 full amino acid sequence |
| mCAR3 | |
| 1308 | mCAR3 scFv |
| 1309 | mCAR3 full amino acid sequence |
| SSJ25-C1 | |
| 1310 | SSJ25-C1 VH sequence |
| 1311 | SSJ25-C1 VL |
| Humanized CAR1 | |
| 1312 | CAR1 scFv domain |
| 1313 | CAR 1 - Full - aa |
| Humanized CAR2 | |
| 1314 | CAR2 scFv domain - aa (Linker is underlined) |
| 1315 | CAR2 scFv domain - nt |
| 1316 | CAR 2 - Full - aa |
| 1317 | CAR 2 - Full - nt |
| 1318 | CAR2 - Soluble scFv - aa |
| Humanized CAR3 | |
| 1319 | CAR3 scFv domain |
| 1320 | CAR 3 - Full - aa |
| Humanized CAR4 | |
| 1321 | CAR4 scFv domain |
| 1322 | CAR 4 - Full - aa |
| Humanized CAR5 | |
| 1323 | CAR5 scFv domain |
| 1324 | CAR 5 - Full - aa |
| Humanized CAR6 | |
| 1325 | CAR6 scFv domain |
| 1326 | CAR6 -Full - aa |
| Humanized CAR7 | |
| 1327 | CAR7 scFv domain |
| 1328 | CAR 7 Full - aa |
| Humanized CAR8 | |
| 1329 | CAR8 scFv domain |
| 1330 | CAR 8 - Full - aa |
| Humanized CAR9 | |
| 1331 | CAR9 scFv domain |
| 1332 | CAR 9 - Full - aa |
| Humanized CAR10 | |
| 1333 | CAR10 scFv domain |
| 1334 | CAR 10 Full - aa |
| Humanized CAR11 | |
| 1335 | CAR11 scFv domain |
| 1336 | CAR 11 Full - aa |
| Humanized CAR12 | |
| 1337 | CAR12 scFv domain |
| 1338 | CAR 12 - Full - aa |
| Murine CART19 | |
| 1339 | HCDR1 (Kabat) |
| 1340 | HCDR2 (Kabat) |
| 1341 | HCDR3 (Kabat) |
| 1342 | LCDR1 (Kabat) |
| 1343 | LCDR2 (Kabat) |
| 1344 | LCDR3 (Kabat) |
| Humanized CART19 a | |
| 1345 | HCDR1 (Kabat) |
| 1346 | HCDR2 (Kabat) |
| 1347 | HCDR3 (Kabat) |
| 1348 | LCDR1 (Kabat) |
| 1349 | LCDR2 (Kabat) |
| 1350 | LCDR3 (Kabat) |
| Humanized CART19 b | |
| 1351 | HCDR1 (Kabat) |
| 1352 | HCDR2 (Kabat) |
| 1353 | HCDR3 (Kabat) |
| 1354 | LCDR1 (Kabat) |
| 1355 | LCDR2 (Kabat) |
| 1356 | LCDR3 (Kabat) |
| Humanized CART19 c | |
| 1357 | HCDR1 (Kabat) |
| 1358 | HCDR2 (Kabat) |
| 1359 | HCDR3 (Kabat) |
| 1360 | LCDR1 (Kabat) |
| 1361 | LCDR2 (Kabat) |
| 1362 | LCDR3 (Kabat) |

CD19 CAR constructs containing humanized anti-CD19 scFv domains are described in PCT publication WO 2014/153270, incorporated herein by reference.

The sequences of murine and humanized CDR sequences of the anti-CD19 scFv domains are shown in Table 9 for the heavy chain variable domains and in Table 10 for the light chain variable domains. The SEQ ID NOs refer to those found in Table 8.

TABLE 9
Heavy Chain Variable Domain CDR (Kabat) SEQ ID NO's of CD19 Antibodies

| Candidate | HCDR1 | HCDR2 | HCDR3 |
|---|---|---|---|
| murine_CART19 | SEQ ID NO: 1339 | SEQ ID NO: 1340 | SEQ ID NO: 1341 |
| humanized_CART19 a | SEQ ID NO: 1345 | SEQ ID NO: 1346 | SEQ ID NO: 1347 |
| humanized_CART19 b | SEQ ID NO: 1351 | SEQ ID NO: 1352 | SEQ ID NO: 1353 |
| humanized_CART19 c | SEQ ID NO: 1357 | SEQ ID NO: 1358 | SEQ ID NO: 1359 |

TABLE 10
Light Chain Variable Domain CDR (Kabat) SEQ ID NO's of CD19 Antibodies

| Candidate | LCDR1 | LCDR2 | LCDR3 |
|---|---|---|---|
| murine_CART19 | SEQ ID NO: 1342 | SEQ ID NO: 1343 | SEQ ID NO: 1344 |
| humanized_CART19 a | SEQ ID NO: 1348 | SEQ ID NO: 1349 | SEQ ID NO: 1350 |
| humanized_CART19 b | SEQ ID NO: 1354 | SEQ ID NO: 1355 | SEQ ID NO: 1356 |
| humanized_CART19 c | SEQ ID NO: 1360 | SEQ ID NO: 1361 | SEQ ID NO: 1362 |

Any known CD19 CAR, e.g., the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Exemplary CD19 CARs include CD19 CARs described herein, e.g., in one or more tables described herein, or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

### CD20 CAR

In some embodiments, the BCMA CAR-expressing cell therapy is administered in combination with a CD20 CAR-expressing cell therapy.

In one embodiment, the CD20 CAR comprises one or more of: LC CDR1, LC CDR2, LC CDR3, HC CDR1, HC CDR2, HC CDR3, VH, VL, an scFv, or full-length sequence of a construct of Tables 11, 12, 13, e.g., CAR20-1, CAR20-2, CAR20-3, CAR20-4, CAR20-5, CAR20-6, CAR20-7, CAR20-8, CAR20-9, CAR20-10, CAR20-11, CAR20-12, CAR20-13, CAR20-14, CAR20-15, or CAR20-16, or a sequence substantially identical thereto (e.g., a sequence sharing 80%, 85%, 90%, or 95% identity thereto). Each full CD20 CAR amino acid sequence in Table 11 includes an optional signal peptide sequence of 21 amino acids corresponding to the amino acid sequence: MALPVTALLLPLALLLHAARP (SEQ ID NO: 2031). Each full CAR nucleotide sequence in Table 11 includes an optional nucleotide signal peptide sequence corresponding to the first 63 nucleotides corresponding to the nucleotide sequence:

(SEQ ID NO: 2032)
ATGGCCCTCCCTGTCACCGCCCTGCTGCTTCCGCTGGCTCTTCTGCTCC
ACGCCGCTCGGCCC.

TABLE 11
CD20 CAR Constructs

| SEQ ID NO: | Ab region |
|---|---|
| CD20-C3H2 | |
| 1363 (Kabat) | HCDR1 |
| 1364 (Kabat) | HCDR2 |
| 1365 (Kabat) | HCDR3 |
| 1366 (Chothia) | HCDR1 |
| 1367 (Chothia) | HCDR2 |
| 1368 (Chothia) | HCDR3 |
| 1369 (IMGT) | HCDR1 |
| 1370 (IMGT) | HCDR2 |
| 1371 (IMGT) | HCDR3 |
| 1372 (Combined Chothia and Kabat) | HCDR1 |
| 1373 (Combined Chothia and Kabat) | HCDR2 |
| 1374 (Combined Chothia and Kabat) | HCDR3 |
| 1375 | VH |
| 1376 | DNA VH |
| 1377 (Kabat) | LCDR1 |
| 1378 (Kabat) | LCDR2 |
| 1379 (Kabat) | LCDR3 |
| 1380 (Chothia) | LCDR1 |
| 1381 (Chothia) | LCDR2 |
| 1382 (Chothia) | LCDR3 |
| 1383 (IMGT) | LCDR1 |
| 1384 (IMGT) | LCDR2 |
| 1385 (IMGT) | LCDR3 |
| 1386 (Combined Chothia and Kabat) | LCDR1 |
| 1387 (Combined Chothia and Kabat) | LCDR2 |
| 1388 (Combined Chothia and Kabat) | LCDR3 |
| 1389 | VL |
| 1390 | DNA VL |
| 1391 | Linker |
| 1392 | scFv (VH-linker-VL) |
| 1393 | DNA scFv (VH-linker-VL) |
| 1394 | Full CAR amino acid sequence |
| 1395 | Full CAR nucleic acid sequence |
| CD20-C5H1 | |
| 1396 (Kabat) | HCDR1 |
| 1397 (Kabat) | HCDR2 |
| 1398 (Kabat) | HCDR3 |
| 1399 (Chothia) | HCDR1 |
| 1400 (Chothia) | HCDR2 |
| 1401 (Chothia) | HCDR3 |
| 1402 (IMGT) | HCDR1 |
| 1403 (IMGT) | HCDR2 |
| 1404 (IMGT) | HCDR3 |
| 1405 (Combined Chothia and Kabat) | HCDR1 |
| 1406 (Combined Chothia and Kabat) | HCDR2 |
| 1407 (Combined Chothia and Kabat) | HCDR3 |
| 1408 | VH |
| 1409 | DNA VH |
| 1410 (Kabat) | LCDR1 |
| 1411 (Kabat) | LCDR2 |
| 1412 (Kabat) | LCDR3 |
| 1413 (Chothia) | LCDR1 |
| 1414 (Chothia) | LCDR2 |
| 1415 (Chothia) | LCDR3 |
| 1416 (IMGT) | LCDR1 |
| 1417 (IMGT) | LCDR2 |
| 1418 (IMGT) | LCDR3 |
| 1419 (Combined Chothia and Kabat) | LCDR1 |
| 1420 (Combined Chothia and Kabat) | LCDR2 |
| 1421 (Combined Chothia and Kabat) | LCDR3 |
| 1422 | VL |
| 1423 | DNA VL |
| 1424 | Linker |
| 1425 | scFv (VH-linker-VL) |
| 1426 | DNA scFv (VH-linker-VL) |
| 1427 | Full CAR amino acid sequence |
| 1428 | Full CAR nucleic acid sequence |
| CD20-C2H1 | |
| 1429 (Kabat) | HCDR1 |
| 1430 (Kabat) | HCDR2 |
| 1431 (Kabat) | HCDR3 |
| 1432 (Chothia) | HCDR1 |
| 1433 (Chothia) | HCDR2 |
| 1434 (Chothia) | HCDR3 |
| 1435 (IMGT) | HCDR1 |
| 1436 (IMGT) | HCDR2 |
| 1437 (IMGT) | HCDR3 |
| 1438 (Combined Chothia and Kabat) | HCDR1 |
| 1439 (Combined Chothia and Kabat) | HCDR2 |
| 1440 (Combined Chothia and Kabat) | HCDR3 |
| 1441 | VH |
| 1442 | DNA VH |
| 1443 (Kabat) | LCDR1 |
| 1444 (Kabat) | LCDR2 |
| 1445 (Kabat) | LCDR3 |
| 1446 (Chothia) | LCDR1 |
| 1447 (Chothia) | LCDR2 |
| 1448 (Chothia) | LCDR3 |
| 1449 (IMGT) | LCDR1 |
| 1450 (IMGT) | LCDR2 |
| 1451 (IMGT) | LCDR3 |
| 1452 (Combined Chothia and Kabat) | LCDR1 |
| 1453 (Combined Chothia and Kabat) | LCDR2 |
| 1454 (Combined Chothia and Kabat) | LCDR3 |
| 1455 | VL |
| 1456 | DNA VL |
| 1457 | Linker |
| 1458 | scFv (VH-linker-VL) |
| 1459 | DNA scFv (VH-linker-VL) |
| 1460 | Full CAR amino acid sequence |
| 1461 | Full CAR nucleic acid sequence |
| CD20-C2H2 | |
| 1462 (Kabat) | HCDR1 |
| 1463 (Kabat) | HCDR2 |
| 1464 (Kabat) | HCDR3 |
| 1465 (Chothia) | HCDR1 |
| 1466 (Chothia) | HCDR2 |
| 1467 (Chothia) | HCDR3 |
| 1468 (IMGT) | HCDR1 |
| 1469 (IMGT) | HCDR2 |
| 1470 (IMGT) | HCDR3 |
| 1471 (Combined Chothia and Kabat) | HCDR1 |
| 1472 (Combined Chothia and Kabat) | HCDR2 |
| 1473 (Combined Chothia and Kabat) | HCDR3 |
| 1474 | VH |
| 1475 | DNA VH |
| 1476 (Kabat) | LCDR1 |
| 1477 (Kabat) | LCDR2 |
| 1478 (Kabat) | LCDR3 |
| 1479 (Chothia) | LCDR1 |
| 1480 (Chothia) | LCDR2 |
| 1481 (Chothia) | LCDR3 |
| 1482 (IMGT) | LCDR1 |
| 1483 (IMGT) | LCDR2 |
| 1484 (IMGT) | LCDR3 |
| 1485 (Combined Chothia and Kabat) | LCDR1 |
| 1486 (Combined Chothia and Kabat) | LCDR2 |
| 1487 (Combined Chothia and Kabat) | LCDR3 |
| 1488 | VL |
| 1489 | DNA VL |
| 1490 | Linker |
| 1491 | scFv (VH-linker-VL) |
| 1492 | DNA scFv (VH-linker-VL) |
| 1493 | Full CAR amino acid sequence |
| 1494 | Full CAR nucleic acid sequence |
| CD20-C2H3 | |
| 1495 (Kabat) | HCDR1 |
| 1496 (Kabat) | HCDR2 |
| 1497 (Kabat) | HCDR3 |
| 1498 (Chothia) | HCDR1 |
| 1499 (Chothia) | HCDR2 |
| 1500 (Chothia) | HCDR3 |
| 1501 (IMGT) | HCDR1 |
| 1502 (IMGT) | HCDR2 |
| 1503 (IMGT) | HCDR3 |
| 1504 (Combined Chothia and Kabat) | HCDR1 |
| 1505 (Combined Chothia and Kabat) | HCDR2 |
| 1506 (Combined Chothia and Kabat) | HCDR3 |
| 1507 | VH |
| 1508 | DNA VH |
| 1509 (Kabat) | LCDR1 |
| 1510 (Kabat) | LCDR2 |
| 1511 (Kabat) | LCDR3 |
| 1512 (Chothia) | LCDR1 |
| 1513 (Chothia) | LCDR2 |
| 1514 (Chothia) | LCDR3 |
| 1515 (IMGT) | LCDR1 |
| 1516 (IMGT) | LCDR2 |
| 1517 (IMGT) | LCDR3 |
| 1518 (Combined Chothia and Kabat) | LCDR1 |
| 1519 (Combined Chothia and Kabat) | LCDR2 |
| 1520 (Combined Chothia and Kabat) | LCDR3 |
| 1521 | VL |
| 1522 | DNA VL |
| 1523 | Linker |
| 1524 | scFv (VH-linker-VL) |
| 1525 | DNA scFv (VH-linker-VL) |
| 1526 | Full CAR amino acid sequence |
| 1527 | Full CAR nucleic acid sequence |
| CD20-C2H4 | |
| 1528 (Kabat) | HCDR1 |
| 1529 (Kabat) | HCDR2 |
| 1530 (Kabat) | HCDR3 |
| 1531 (Chothia) | HCDR1 |
| 1532 (Chothia) | HCDR2 |
| 1533 (Chothia) | HCDR3 |
| 1534 (IMGT) | HCDR1 |
| 1535 (IMGT) | HCDR2 |
| 1536 (IMGT) | HCDR3 |
| 1537 (Combined Chothia and Kabat) | HCDR1 |
| 1538 (Combined Chothia and Kabat) | HCDR2 |
| 1539 (Combined Chothia and Kabat) | HCDR3 |
| 1540 | VH |
| 1541 | DNA VH |
| 1542 (Kabat) | LCDR1 |
| 1543 (Kabat) | LCDR2 |
| 1544 (Kabat) | LCDR3 |
| 1545 (Chothia) | LCDR1 |
| 1546 (Chothia) | LCDR2 |
| 1547 (Chothia) | LCDR3 |
| 1548 (IMGT) | LCDR1 |
| 1549 (IMGT) | LCDR2 |
| 1550 (IMGT) | LCDR3 |
| 1551 (Combined Chothia and Kabat) | LCDR1 |
| 1552 (Combined Chothia and Kabat) | LCDR2 |
| 1553 (Combined Chothia and Kabat) | LCDR3 |
| 1554 | VL |
| 1555 | DNA VL |
| 1556 | Linker |
| 1557 | scFv (VH-linker-VL) |
| 1558 | DNA scFv (VH-linker-VL) |
| 1559 | Full CAR amino acid sequence |
| 1560 | Full CAR nucleic acid sequence |
| CD20-C3H1 | |
| 1561 (Kabat) | HCDR1 |
| 1562 (Kabat) | HCDR2 |
| 1563 (Kabat) | HCDR3 |
| 1564 (Chothia) | HCDR1 |
| 1565 (Chothia) | HCDR2 |
| 1566 (Chothia) | HCDR3 |
| 1567 (IMGT) | HCDR1 |
| 1568 (IMGT) | HCDR2 |
| 1569 (IMGT) | HCDR3 |
| 1570 (Combined Chothia and Kabat) | HCDR1 |
| 1571 (Combined Chothia and Kabat) | HCDR2 |
| 1572 (Combined Chothia and Kabat) | HCDR3 |
| 1573 | VH |
| 1574 | DNA VH |
| 1575 (Kabat) | LCDR1 |
| 1576 (Kabat) | LCDR2 |
| 1577 (Kabat) | LCDR3 |
| 1578 (Chothia) | LCDR1 |
| 1579 (Chothia) | LCDR2 |
| 1580 (Chothia) | LCDR3 |
| 1581 (IMGT) | LCDR1 |
| 1582 (IMGT) | LCDR2 |
| 1583 (IMGT) | LCDR3 |
| 1584 (Combined Chothia and Kabat) | LCDR1 |
| 1585 (Combined Chothia and Kabat) | LCDR2 |
| 1586 (Combined Chothia and Kabat) | LCDR3 |
| 1587 | VL |
| 1588 | DNA VL |
| 1589 | Linker |
| 1590 | scFv (VH-linker-VL) |
| 1591 | DNA scFv (VH-linker-VL) |
| 1592 | Full CAR amino acid sequence |
| 1593 | Full CAR nucleic acid sequence |
| CD20-C3H3 | |
| 1594 (Kabat) | HCDR1 |
| 1595 (Kabat) | HCDR2 |
| 1596 (Kabat) | HCDR3 |
| 1597 (Chothia) | HCDR1 |
| 1598 (Chothia) | HCDR2 |
| 1599 (Chothia) | HCDR3 |
| 1600 (IMGT) | HCDR1 |
| 1601 (IMGT) | HCDR2 |
| 1602 (IMGT) | HCDR3 |
| 1603 | VH |
| 1604 | DNA VH |
| 1605 (Kabat) | LCDR1 |
| 1606 (Kabat) | LCDR2 |
| 1607 (Kabat) | LCDR3 |
| 1608 (Chothia) | LCDR1 |
| 1609 (Chothia) | LCDR2 |
| 1610 (Chothia) | LCDR3 |
| 1611 (IMGT) | LCDR1 |
| 1612 (IMGT) | LCDR2 |
| 1613 (IMGT) | LCDR3 |
| 1614 (Combined Chothia and Kabat) | LCDR1 |
| 1615 (Combined Chothia and Kabat) | LCDR2 |
| 1616 (Combined Chothia and Kabat) | LCDR3 |
| 1617 | VL |
| 1618 | DNA VL |
| 1619 | Linker |
| 1620 | scFv (VH-linker-VL) |
| 1621 | DNA scFv (VH-linker-VL) |
| 1622 | Full CAR amino acid sequence |
| 1623 | Full CAR nucleic acid sequence |
| CD20-C3H4 | |
| 1624 (Kabat) | HCDR1 |
| 1625 (Kabat) | HCDR2 |
| 1626 (Kabat) | HCDR3 |
| 1627 (Chothia) | HCDR1 |
| 1628 (Chothia) | HCDR2 |
| 1629 (Chothia) | HCDR3 |
| 1630 (IMGT) | HCDR1 |
| 1631 (IMGT) | HCDR2 |
| 1632 (IMGT) | HCDR3 |
| 1633 (Combined Chothia and Kabat) | HCDR1 |
| 1634 (Combined Chothia and Kabat) | HCDR2 |
| 1635 (Combined Chothia and Kabat) | HCDR3 |
| 1636 | VH |
| 1637 | DNA VH |
| 1638 (Kabat) | LCDR1 |
| 1639 (Kabat) | LCDR2 |
| 1640 (Kabat) | LCDR3 |
| 1641 (Chothia) | LCDR1 |
| 1642 (Chothia) | LCDR2 |
| 1643 (Chothia) | LCDR3 |
| 1644 (IMGT) | LCDR1 |
| 1645 (IMGT) | LCDR2 |
| 1646 (IMGT) | LCDR3 |
| 1647 (Combined Chothia and Kabat) | LCDR1 |
| 1648 (Combined Chothia and Kabat) | LCDR2 |
| 1649 (Combined Chothia and Kabat) | LCDR3 |
| 1650 | VL |
| 1651 | DNA VL |
| 1652 | Linker |
| 1653 | scFv (VH-linker-VL) |
| 1654 | DNA scFv (VH-linker-VL) |
| 1655 | Full CAR amino acid sequence |
| 1656 | Full CAR nucleic acid sequence |
| CD20-C5H2 | |
| 1657 (Kabat) | HCDR1 |
| 1658 (Kabat) | HCDR2 |
| 1659 (Kabat) | HCDR3 |
| 1660 (Chothia) | HCDR1 |
| 1661 (Chothia) | HCDR2 |
| 1662 (Chothia) | HCDR3 |
| 1663 (IMGT) | HCDR1 |
| 1664 (IMGT) | HCDR2 |
| 1665 (IMGT) | HCDR3 |
| 1666 (Combined Chothia and Kabat) | HCDR1 |
| 1667 (Combined Chothia and Kabat) | HCDR2 |
| 1668 (Combined Chothia and Kabat) | HCDR3 |
| 1669 | VH |
| 1670 | DNA VH |
| 1671 (Kabat) | LCDR1 |
| 1672 (Kabat) | LCDR2 |
| 1673 (Kabat) | LCDR3 |
| 1674 (Chothia) | LCDR1 |
| 1675 (Chothia) | LCDR2 |
| 1676 (Chothia) | LCDR3 |
| 1677 (IMGT) | LCDR1 |
| 1678 (IMGT) | LCDR2 |
| 1679 (IMGT) | LCDR3 |
| 1680 (Combined Chothia and Kabat) | LCDR1 |
| 1681 (Combined Chothia and Kabat) | LCDR2 |
| 1682 (Combined Chothia and Kabat) | LCDR3 |
| 1683 | VL |
| 1684 | DNA VL |
| 1685 | Linker |
| 1686 | scFv (VH-linker-VL) |
| 1687 | DNA scFv (VH-linker-VL) |
| 1688 | Full CAR amino acid sequence |
| 1689 | Full CAR nucleic acid sequence |
| CD20-C5H3 | |
| 1690 (Kabat) | HCDR1 |
| 1691 (Kabat) | HCDR2 |
| 1692 (Kabat) | HCDR3 |
| 1693 (Chothia) | HCDR1 |
| 1694 (Chothia) | HCDR2 |
| 1695 (Chothia) | HCDR3 |
| 1696 (IMGT) | HCDR1 |
| 1697 (IMGT) | HCDR2 |
| 1698 (IMGT) | HCDR3 |
| 1699 (Combined Chothia and Kabat) | HCDR1 |
| 1700 (Combined Chothia and Kabat) | HCDR2 |
| 1701 (Combined Chothia and Kabat) | HCDR3 |
| 1702 | VH |
| 1703 | DNA VH |
| 1704 (Kabat) | LCDR1 |
| 1705 (Kabat) | LCDR2 |
| 1706 (Kabat) | LCDR3 |
| 1707 (Chothia) | LCDR1 |
| 1708 (Chothia) | LCDR2 |
| 1709 (Chothia) | LCDR3 |
| 1710 (IMGT) | LCDR1 |
| 1711 (IMGT) | LCDR2 |
| 1712 (IMGT) | LCDR3 |
| 1713 (Combined Chothia and Kabat) | LCDR1 |
| 1714 (Combined Chothia and Kabat) | LCDR2 |
| 1715 (Combined Chothia and Kabat) | LCDR3 |
| 1716 | VL |
| 1717 | DNA VL |
| 1718 | Linker |
| 1719 | scFv (VH-linker-VL) |
| 1720 | DNA scFv (VH-linker-VL) |
| 1721 | Full CAR amino acid sequence |
| 1722 | Full CAR nucleic acid sequence |
| CD20-C5H4 | |
| 1723 (Kabat) | HCDR1 |
| 1724 (Kabat) | HCDR2 |
| 1725 (Kabat) | HCDR3 |
| 1726 (Chothia) | HCDR1 |
| 1727 (Chothia) | HCDR2 |
| 1728 (Chothia) | HCDR3 |
| 1729 (IMGT) | HCDR1 |
| 1730 (IMGT) | HCDR2 |
| 1731 (IMGT) | HCDR3 |
| 1732 (Combined Chothia and Kabat) | HCDR1 |
| 1733 (Combined Chothia and Kabat) | HCDR2 |
| 1734 (Combined Chothia and Kabat) | HCDR3 |
| 1735 | VH |
| 1736 | DNA VH |
| 1737 (Kabat) | LCDR1 |
| 1738 (Kabat) | LCDR2 |
| 1739 (Kabat) | LCDR3 |
| 1740 (Chothia) | LCDR1 |
| 1741 (Chothia) | LCDR2 |
| 1742 (Chothia) | LCDR3 |
| 1743 (IMGT) | LCDR1 |
| 1744 (IMGT) | LCDR2 |
| 1745 (IMGT) | LCDR3 |
| 1746 (Combined Chothia and Kabat) | LCDR1 |
| 1747 (Combined Chothia and Kabat) | LCDR2 |
| 1748 (Combined Chothia and Kabat) | LCDR3 |
| 1749 | VL |
| 1750 | DNA VL |
| 1751 | Linker |
| 1752 | scFv (VH-linker-VL) |
| 1753 | DNA scFv (VH-linker-VL) |
| 1754 | Full CAR amino acid sequence |
| 1755 | Full CAR nucleic acid sequence |
| CD20-C8H1 | |
| 1756 (Kabat) | HCDR1 |
| 1757 (Kabat) | HCDR2 |
| 1758 (Kabat) | HCDR3 |
| 1759 (Chothia) | HCDR1 |
| 1760 (Chothia) | HCDR2 |
| 1761 (Chothia) | HCDR3 |
| 1762 (IMGT) | HCDR1 |
| 1763 (IMGT) | HCDR2 |
| 1764 (IMGT) | HCDR3 |
| 1765 (Combined Chothia and Kabat) | HCDR1 |
| 1766 (Combined Chothia and Kabat) | HCDR2 |
| 1767 (Combined Chothia and Kabat) | HCDR3 |
| 1768 | VH |
| 1769 | DNA VH |
| 1770 (Kabat) | LCDR1 |
| 1771 (Kabat) | LCDR2 |
| 1772 (Kabat) | LCDR3 |
| 1773 (Chothia) | LCDR1 |
| 1774 (Chothia) | LCDR2 |
| 1775 (Chothia) | LCDR3 |
| 1776 (IMGT) | LCDR1 |
| 1777 (IMGT) | LCDR2 |
| 1778 (IMGT) | LCDR3 |
| 1779 (Combined Chothia and Kabat) | LCDR1 |
| 1780 (Combined Chothia and Kabat) | LCDR2 |
| 1781 (Combined Chothia and Kabat) | LCDR3 |
| 1782 | VL |
| 1783 | DNA VL |
| 1784 | Linker |
| 1785 | scFv (VH-linker-VL) |
| 1786 | DNA scFv (VH-linker-VL) |
| 1787 | Full CAR amino acid sequence |
| 1788 | Full CAR nucleic acid sequence |
| CD20-C8H2 | |
| 1789 (Kabat) | HCDR1 |
| 1790 (Kabat) | HCDR2 |
| 1791 (Kabat) | HCDR3 |
| 1792 (Chothia) | HCDR1 |
| 1793 (Chothia) | HCDR2 |
| 1794 (Chothia) | HCDR3 |
| 1795 (IMGT) | HCDR1 |
| 1796 (IMGT) | HCDR2 |
| 1797 (IMGT) | HCDR3 |
| 1798 (Combined Chothia and Kabat) | HCDR1 |
| 1799 (Combined Chothia and Kabat) | HCDR2 |
| 1800 (Combined Chothia and Kabat) | HCDR3 |
| 1801 | VH |
| 1802 | DNA VH |
| 1803 (Kabat) | LCDR1 |
| 1804 (Kabat) | LCDR2 |
| 1805 (Kabat) | LCDR3 |
| 1806 (Chothia) | LCDR1 |
| 1807 (Chothia) | LCDR2 |
| 1808 (Chothia) | LCDR3 |
| 1809 (IMGT) | LCDR1 |
| 1810 (IMGT) | LCDR2 |
| 1811 (IMGT) | LCDR3 |
| 1812 (Combined Chothia and Kabat) | LCDR1 |
| 1813 (Combined Chothia and Kabat) | LCDR2 |
| 1814 (Combined Chothia and Kabat) | LCDR3 |
| 1815 | VL |
| 1816 | DNA VL |
| 1817 | Linker |
| 1818 | scFv (VH-linker-VL) |
| 1819 | DNA scFv (VH-linker-VL) |
| 1820 | Full CAR amino acid sequence |
| 1821 | Full CAR nucleic acid sequence |
| CD20-C8H3 | |
| 1822 (Kabat) | HCDR1 |
| 1823 (Kabat) | HCDR2 |
| 1824 (Kabat) | HCDR3 |
| 1825 (Chothia) | HCDR1 |
| 1826 (Chothia) | HCDR2 |
| 1827 (Chothia) | HCDR3 |
| 1828 (IMGT) | HCDR1 |
| 1829 (IMGT) | HCDR2 |
| 1830 (IMGT) | HCDR3 |
| 1831 (Combined Chothia and Kabat) | HCDR1 |
| 1832 (Combined Chothia and Kabat) | HCDR2 |
| 1833 (Combined Chothia and Kabat) | HCDR3 |
| 1834 | VH |
| 1835 | DNA VH |
| 1836 (Kabat) | LCDR1 |
| 1837 (Kabat) | LCDR2 |
| 1838 (Kabat) | LCDR3 |
| 1839 (Chothia) | LCDR1 |
| 1840 (Chothia) | LCDR2 |
| 1841 (Chothia) | LCDR3 |
| 1842 (IMGT) | LCDR1 |
| 1843 (IMGT) | LCDR2 |
| 1844 (IMGT) | LCDR3 |
| 1845 (Combined Chothia and Kabat) | LCDR1 |
| 1846 (Combined Chothia and Kabat) | LCDR2 |
| 1847 (Combined Chothia and Kabat) | LCDR3 |
| 1848 | VL |
| 1849 | DNA VL |
| 1850 | Linker |
| 1851 | scFv (VH-linker-VL) |
| 1852 | DNA scFv (VH-linker-VL) |
| 1853 | Full CAR amino acid sequence |
| 1854 | Full CAR nucleic acid sequence |
| CD20-C8H4 | |
| 1855 (Kabat) | HCDR1 |
| 1856 (Kabat) | HCDR2 |
| 1857 (Kabat) | HCDR3 |
| 1858 (Chothia) | HCDR1 |
| 1859 (Chothia) | HCDR2 |
| 1860 (Chothia) | HCDR3 |
| 1861 (IMGT) | HCDR1 |
| 1862 (IMGT) | HCDR2 |
| 1863 (IMGT) | HCDR3 |
| 1864 (Combined Chothia and Kabat) | HCDR1 |
| 1865 (Combined Chothia and Kabat) | HCDR2 |
| 1866 (Combined Chothia and Kabat) | HCDR3 |
| 1867 | VH |
| 1868 | DNA VH |
| 1869 (Kabat) | LCDR1 |
| 1870 (Kabat) | LCDR2 |
| 1871 (Kabat) | LCDR3 |
| 1872 (Chothia) | LCDR1 |
| 1873 (Chothia) | LCDR2 |
| 1874 (Chothia) | LCDR3 |
| 1875 (IMGT) | LCDR1 |
| 1876 (IMGT) | LCDR2 |
| 1877 (IMGT) | LCDR3 |
| 1878 (Combined Chothia and Kabat) | LCDR1 |
| 1879 (Combined Chothia and Kabat) | LCDR2 |
| 1880 (Combined Chothia and Kabat) | LCDR3 |
| 1881 | VL |
| 1882 | DNA VL |
| 1883 | Linker |
| 1884 | scFv (VH-linker-VL) |
| 1885 | DNA scFv (VH-linker-VL) |
| 1886 | Full CAR amino acid sequence |
| 1887 | Full CAR nucleic acid sequence |
| CD20-C2 | |
| 1888 | VH |
| 1889 | DNA VH |
| 1890 | VL |
| 1891 | DNA VL |
| CD20-C3 | |
| 1892 | VH |
| 1893 | DNA VH |
| 1894 | VL |
| 1895 | DNA VL |
| CD20-C5 | |
| 1896 | VH |
| 1897 | DNA VH |
| 1898 | VL |
| 1899 | DNA VL |
| CD20-C6 | |
| 1900 | VH |
| 1901 | DNA VH |
| 1902 | VL |
| 1903 | DNA VL |
| CD20-C7 | |
| 1904 | VH |
| 1905 | DNA VH |
| 1906 | VL |
| 1907 | DNA VL |
| CD20-C8 | |
| 1908 | VH |
| 1909 | DNA VH |
| 1910 | VL |
| 1911 | DNA VL |
| CD20-3m | |
| 1912 | VH |
| 1913 | DNA VH |
| 1914 | VL |
| 1915 | DNA VL |
| 1916 | Linker |
| 1917 | scFv (VH-linker-VL) |
| CD20-3J | |
| 1918 | VH |
| 1919 | DNA VH |
| 1920 | VL |
| 1921 | DNA VL |
| 1922 | Linker |
| 1923 | scFv (VH-linker-VL) |
| CD20-3H5k1 | |
| 1924 | VH |
| 1925 | DNA VH |
| 1926 | VL |
| 1927 | DNA VL |
| 1928 | Linker |
| 1929 | scFv (VH-linker-VL) |
| CD20-3H5k3 | |
| 1930 | VH |
| 1931 | DNA VH |
| 1932 | VL |
| 1933 | DNA VL |
| 1934 | Linker |
| 1935 | scFv (VH-linker-VL) |
| CD20-Ofa | |
| 1936 (Kabat) | HCDR1 |
| 1937 (Kabat) | HCDR2 |
| 1938 (Kabat) | HCDR3 |
| 1939 (Chothia) | HCDR1 |
| 1940 (Chothia) | HCDR2 |
| 1941 (Chothia) | HCDR3 |
| 1942 (IMGT) | HCDR1 |
| 1943 (IMGT) | HCDR2 |
| 1944 (IMGT) | HCDR3 |
| 1945 | VH |
| 1946 | DNA VH |
| 1947 (Kabat) | LCDR1 |
| 1948 (Kabat) | LCDR2 |
| 1949 (Kabat) | LCDR3 |
| 1950 (Chothia) | LCDR1 |
| 1951 (Chothia) | LCDR2 |
| 1952 (Chothia) | LCDR3 |
| 1953 (IMGT) | LCDR1 |
| 1954 (IMGT) | LCDR2 |
| 1955 (IMGT) | LCDR3 |
| 1956 | VL |
| 1957 | DNA VL |
| 1958 | Linker |
| 1959 | scFv (VH-linker-VL) |
| 1960 | DNA scFv (VH-linker-VL) |
| CD20-3 | |
| 1961 | VH |
| 1962 | VL |
| 1963 | Linker |
| 1964 | scFv (VH-linker-VL) |
| CD20-8aBBz | |
| 1965 | VH |
| 1966 | DNA VH |
| 1967 | VL |
| 1968 | DNA VL |
| 1969 | Linker |
| 1970 | scFv (VH-linker-VL) |
| 1971 | DNA scFv (VH-linker-VL) |

An overview of the sequences identifications of CDR (Kabat) sequences of the CD20 scFv domains of Table 11 are shown in Table 12 for the heavy chain variable domains and in Table 13 for the light chain variable domains. The SEQ ID NOs refer to those found in Table 11.

TABLE 12
Heavy Chain Variable Domain CDR (Kabat)
SEQ ID NO's of CD20 CAR molecules

| Candidate | HCDR1 | HCDR2 | HCDR3 |
|---|---|---|---|
| CD20-C3H2 | 1363 | 1364 | 1365 |
| CD20-C5H1 | 1396 | 1397 | 1398 |
| CD20-C2H1 | 1429 | 1430 | 1431 |
| CD20-C2H2 | 1462 | 1463 | 1464 |
| CD20-C2H3 | 1495 | 1496 | 1497 |
| CD20-C2H4 | 1528 | 1529 | 1530 |
| CD20-C3H1 | 1561 | 1562 | 1563 |
| CD20-C3H3 | 1594 | 1595 | 1596 |
| CD20-C3H4 | 1624 | 1625 | 1626 |
| CD20-C5H2 | 1657 | 1658 | 1659 |
| CD20-C5H3 | 1690 | 1691 | 1692 |
| CD20-C5H4 | 1723 | 1724 | 1725 |
| CD20-C8H1 | 1756 | 1757 | 1758 |
| CD20-C8H2 | 1789 | 1790 | 1791 |
| CD20-C8H3 | 1822 | 1823 | 1824 |
| CD20-C8H4 | 1855 | 1856 | 1857 |

TABLE 13
Light Chain Variable Domain CDR (Kabat)
SEQ ID NO's of CD20 Antibody Molecules

| Candidate | LCDR1 | LCDR2 | LCDR3 |
|---|---|---|---|
| CD20-C3H2 | 1377 | 1378 | 1379 |
| CD20-C5H1 | 1410 | 1411 | 1412 |
| CD20-C2H1 | 1443 | 1444 | 1445 |
| CD20-C2H2 | 1476 | 1477 | 1478 |
| CD20-C2H3 | 1509 | 1510 | 1511 |
| CD20-C2H4 | 1542 | 1543 | 1544 |
| CD20-C3H1 | 1575 | 1576 | 1577 |
| CD20-C3H3 | 1605 | 1606 | 1607 |
| CD20-C3H4 | 1638 | 1639 | 1640 |
| CD20-C5H2 | 1671 | 1672 | 1673 |
| CD20-C5H3 | 1704 | 1705 | 1706 |
| CD20-C5H4 | 1737 | 1738 | 1739 |
| CD20-C8H1 | 1770 | 1771 | 1772 |
| CD20-C8H2 | 1803 | 1804 | 1805 |
| CD20-C8H3 | 1836 | 1837 | 1838 |
| CD20-C8H4 | 1869 | 1870 | 1871 |

### Additional CD20 Inhibitors

In some embodiments, the BCMA CAR-expressing cell therapy is administered in combination with a CD20 inhibitor.

In one embodiment, the CD20 inhibitor is an anti-CD20 antibody or fragment thereof. In an embodiment, the antibody is a monospecific antibody and in another embodiment the antibody is a bispecific antibody. In an embodiment, the CD20 inhibitor is a chimeric mouse/human monoclonal antibody, e.g., rituximab. In an embodiment, the CD20 inhibitor is a human monoclonal antibody such as of atumumab. In an embodiment, the CD20 inhibitor is a humanized antibody such as ocrelizumab, veltuzumab, obinutuzumab, ocaratuzumab, or PRO131921 (Genentech). In an embodiment, the CD20 inhibitor is a fusion protein comprising a portion of an anti-CD20 antibody, such as TRU-015 (Trubion Pharmaceuticals).

### CD22 CAR

In some embodiments, the BCMA CAR-expressing cell therapy is administered in combination with a CD22 CAR-expressing cell therapy (e.g., cells expressing a CAR that binds to human CD22).

In some embodiments, the CD22 CAR-expressing cell therapy includes an antigen binding domain according to WO2016/164731, incorporated herein by reference.

The sequences of CD22 CAR are provided below. In some embodiments, the CD22 CAR is CD22-65. In some embodiments, the CD22 CAR is CD22-65s. In some embodiments, the CD22 CAR is CD22-65ss.

Human CD22 CAR CD22-65 scFv sequence
(SEQ ID NO: 1972)
EVQLQQSGPGLVKPSQTLSLTCAISGDSMLSNSDTWNWIRQSPSRGLEW
LGRTYHRSTWYDDYASSVRGRVSINVDTSKNQYSLQLNAVTPEDTGVYY
CARVRLQDGNSWSDAFDVWGQGTMVTVSSGGGGSGGGGSGGGGSQSALT
QPASASGSPGQSVTISCTGTSSDVGGYNYVSWYQQHPGKAPKLMIYDVS
NRPSGVSNRFSGSKSGNTASLTISGLQAEDEADYYCSSYTSSSTLYVFG
TGTQLTVL
Human CD22 CAR CD22-65s scFc sequence (linker
shown by italics and underline)
(SEQ ID NO: 2036)
EVQLQQSGPGLVKPSQTLSLTCAISGDSMLSNSDTWNWIRQSPSRGLEW
LGRTYHRSTWYDDYASSVRGRVSINVDTSKNQYSLQLNAVTPEDTGVYY
CARVRLQDGNSWSDAFDVWGQGTMVTVSSGGGGSQSALTQPASASGSPG
QSVTISCTGTSSDVGGYNYVSWYQQHPGKAPKLMIYDVSNRPSGVSNRF
SGSKSGNTASLTISGLQAEDEADYYCSSYTSSSTLYVFGTGTQLTVL
Human CD22 CAR CD22-65ss scFc sequence
(SEQ ID NO: 1973)
EVQLQQSGPGLVKPSQTLSLTCAISGDSMLSNSDTWNWIRQSPSRGLEW
LGRTYHRSTWYDDYASSVRGRVSINVDTSKNQYSLQLNAVTPEDTGVYY
CARVRLQDGNSWSDAFDVWGQGTMVTVSSQSALTQPASASGSPGQSVTI
SCTGTSSDVGGYNYVSWYQQHPGKAPKLMIYDVSNRPSGVSNRFSGSKS
GNTASLTISGLQAEDEADYYCSSYTSSSTLYVFGTGTQLTVL
Human CD22 CAR heavy chain variable region
(SEQ ID NO: 1974)
EVQLQQSGPGLVKPSQTLSLTCAISGDSMLSNSDTWNWIRQSPSRGLEW
LGRTYHRSTWYDDYASSVRGRVSINVDTSKNQYSLQLNAVTPEDTGVYY
CARVRLQDGNSWSDAFDVWGQGTMVTVSS
Human CD22 CAR light chain variable region
(SEQ ID NO: 1975)
QSALTQPASASGSPGQSVTISCTGTSSDVGGYNYVSWYQQHPGKAPKLM
IYDVSNRPSGVSNRFSGSKSGNTASLTISGLQAEDEADYYCSSYTSSST
LYVFGTGTQLTVL

TABLE 14
Heavy Chain Variable Domain CDRs of CD22 CAR (CD22-65)

| | | SEQ ID | | SEQ ID | | SEQ ID |
|---|---|---|---|---|---|---|
| Candidate | HCDR1 | NO: | HCDR2 | NO: | HCDR3 | NO: |
| CD22-65 | GDSML | 1976 | RTYHRSTWYDDYA | 1978 | VRLQDGNSWSD | 1980 |
| Combined | SNSDT | | SSVRG | | AFDV | |
| | WN | | | | | |
| CD22-65 | SNSDT | 1977 | RTYHRSTWYDDYA | 1979 | VRLQDGNSWSD | 1981 |
| Kabat | WN | | SSVRG | | AFDV | |

TABLE 15
Light Chain Variable Domain CDRs of CD22 CAR (CD22-65). The LC CDR sequences
in this table have the same sequence under the Kabat or combined definitions.

| | | SEQ ID | | SEQ ID | | SEQ ID |
|---|---|---|---|---|---|---|
| Candidate | LCDR1 | NO: | LCDR2 | NO: | LCDR3 | NO: |
| CD22-65 | TGTSSDVGGYNYVS | 1982 | DVSNRPS | 1983 | SSYTSSSTLYV | 1984 |
| Combined | | | | | | |

In some embodiments, the antigen binding domain comprises a HC CDR1, a HC CDR2, and a HC CDR3 of any heavy chain binding domain amino acid sequences listed in Table 14. In embodiments, the antigen binding domain further comprises a LC CDR1, a LC CDR2, and a LC CDR3. In embodiments, the antigen binding domain comprises a LC CDR1, a LC CDR2, and a LC CDR3 amino acid sequences listed in Table 15.

In some embodiments, the antigen binding domain comprises one, two or all of LC CDR1, LC CDR2, and LC CDR3 of any light chain binding domain amino acid sequences listed in Table 15, and one, two or all of HC CDR1, HC CDR2, and HC CDR3 of any heavy chain binding domain amino acid sequences listed in Table 14.

In some embodiments, the CDRs are defined according to the Kabat numbering scheme, the Chothia numbering scheme, or a combination thereof.

The order in which the VL and VH domains appear in the scFv can be varied (i.e., VL-VH, or VH-VL orientation), and where any of one, two, three or four copies of the “G4S” (SEQ ID NO: 1032) subunit, in which each subunit comprises the sequence GGGGS (SEQ ID NO: 1032) (e.g., (G4S)₃ (SEQ ID NO: 1040) or (G45)₄ (SEQ ID NO: 1039)), can connect the variable domains to create the entirety of the scFv domain. Alternatively, the CAR construct can include, for example, a linker including the sequence GSTSGSGKPGSGEGSTKG (SEQ ID NO: 1985). Alternatively, the CAR construct can include, for example, a linker including the sequence LAEAAAK (SEQ ID NO: 2033). In an embodiment, the CAR construct does not include a linker between the VL and VH domains.

These clones all contained a Q/K residue change in the signal domain of the co-stimulatory domain derived from CD3zeta chain.

### Additional CD22 Inhibitors

In some embodiments, the BCMA CAR-expressing cell therapy is administered in combination with a CD20 inhibitor. In some embodiments, the CD20 inhibitor is a small molecule or an anti-CD20 antibody molecule.

In an embodiment, the antibody is a monospecific antibody, optionally conjugated to a second agent such as a chemotherapeutic agent. For instance, in an embodiment the antibody is an anti-CD22 monoclonal antibody-MMAE conjugate (e.g., DCDT2980S). In an embodiment, the antibody is an scFv of an anti-CD22 antibody, e.g., an scFv of antibody RFB4. This scFv can be fused to all of or a fragment of Pseudomonas exotoxin-A (e.g., BL22). In an embodiment, the antibody is a humanized anti-CD22 monoclonal antibody (e.g., epratuzumab). In an embodiment, the antibody or fragment thereof comprises the Fv portion of an anti-CD22 antibody, which is optionally covalently fused to all or a fragment or (e.g., a 38 KDa fragment of) Pseudomonas exotoxin-A (e.g., moxetumomab pasudotox). In an embodiment, the anti-CD22 antibody is an anti-CD19/CD22 bispecific antibody, optionally conjugated to a toxin. For instance, in one embodiment, the anti-CD22 antibody comprises an anti-CD19/CD22 bispecific portion, (e.g., two scFv ligands, recognizing human CD19 and CD22) optionally linked to all of or a portion of diphtheria toxin (DT), e.g., first 389 amino acids of diphtheria toxin (DT), DT 390, e.g., a ligand-directed toxin such as DT2219ARL). In another embodiment, the bispecific portion (e.g., anti-CD19/anti-CD22) is linked to a toxin such as deglycosylated ricin A chain (e.g., Combotox).

In some embodiments, the CD22 inhibitor is a multispecific antibody molecule, e.g., a bispecific antibody molecule, e.g., a bispecific antibody molecule that binds to CD20 and CD3. Exemplary bispecific antibody molecules that bind to CD20 and CD3 are disclosed in WO2016086189 and WO2016182751, herein incorporated by reference in their entirety. In some embodiments, the bispecific antibody molecule that binds to CD20 and CD3 is XENP13676 as disclosed in FIG. 74, SEQ ID NOs: 323, 324, and 325 of WO2016086189.

### Multispecific CAR

In some embodiments, the CAR molecule disclosed herein is a multispecific, e.g., bispecific, CAR molecule comprising one, two, or more binding specificities, e.g., a first binding specificity for a first antigen, e.g., a B-cell epitope, and a second binding specificity for the same or a different antigen, e.g., B cell epitope.

In one embodiment, the first and second binding specificity is an antibody molecule, e.g., an antigen binding domain (e.g., a scFv). Within each antibody molecule (e.g., scFv) of a bispecific CAR molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific CAR molecule has the arrangement VH₁-VL₁-VL₂-VH₂, from an N- to C-terminal orientation.

In some embodiments, the upstream antibody or antibody fragment or antigen binding domain (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific CAR molecule has the arrangement VL₁-VH₁-VH₂-VL₂, from an N- to C-terminal orientation.

In some embodiments, the upstream antibody or antibody fragment (e.g., scFv) is arranged with its VL (VL₁) upstream of its VH (VH₁) and the downstream antibody or antibody fragment or antigen binding domain (e.g., scFv) is arranged with its VL (VL₂) upstream of its VH (VH₂), such that the overall bispecific CAR molecule has the arrangement VL₁-VH₁-VL₂-VH₂, from an N- to C-terminal orientation.

In some embodiments, the upstream antibody or antibody fragment or antigen binding domain (e.g., scFv) is arranged with its VH (VH₁) upstream of its VL (VL₁) and the downstream antibody or antibody fragment (e.g., scFv) is arranged with its VH (VH₂) upstream of its VL (VL₂), such that the overall bispecific CAR molecule has the arrangement VH₁-VL₁-VH₂-VL₂, from an N- to C-terminal orientation.

In any of the aforesaid configurations, optionally, a linker is disposed between the two antibodies or antibody fragments or antigen binding domains (e.g., scFvs), e.g., between VL₁ and VL₂ if the construct is arranged as VH₁-VL₁-VL₂-VH₂; between VH₁ and VH₂ if the construct is arranged as VL₁-VH₁-VH₂-VL₂; between VH₁ and VL₂ if the construct is arranged as VL₁-VH₁-VL₂-VH₂; or between VL₁ and VH₂ if the construct is arranged as VH₁-VL₁-VH₂-VL₂. In general, the linker between the two antibody fragments or antigen binding domains, e.g., scFvs, should be long enough to avoid mispairing between the domains of the two scFvs. The linker may be a linker as described herein. In some embodiments, the linker is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In some embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the linker comprises, e.g., consists of, the amino acid sequence: LAEAAAK (SEQ ID NO: 2033).

In any of the aforesaid configurations, optionally, a linker is disposed between the VL and VH of the first antigen binding domains, e.g., scFv. Optionally, a linker is disposed between the VL and VH of the second antigen binding domains, e.g., scFv. In constructs that have multiple linkers, any two or more of the linkers can be the same or different. Accordingly, in some embodiments, a bispecific CAR comprises VLs, VHs, and optionally one or more linkers in an arrangement as described herein.

In some embodiments, each antibody molecule, e.g., each antigen binding domain (e.g., each scFv) comprises a linker between the VH and the VL regions. In some embodiments, the linker between the VH and the VL regions is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In other embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the VH and VL regions are connected without a linker.

In certain embodiments, the CAR molecule is a bispecific CAR molecule having a first binding specificity for a first B-cell epitope and a second binding specificity for the same or a different B-cell antigen. For instance, in some embodiments the bispecific CAR molecule has a first binding specificity for BCMA and a second binding specificity for one or more of BCMA, CD10, CD19, CD20, CD22, CD34, CD123, FLT-3, ROR1, CD79b, CD179b, or CD79a. In some embodiments the bispecific CAR molecule has a first binding specificity for BCMA and a second binding specificity for CD19. In some embodiments the bispecific CAR molecule has a first binding specificity for BCMA and a second binding specificity for CD20. In some embodiments the bispecific CAR molecule has a first binding specificity for BCMA and a second binding specificity for CD22.

In one embodiment, the CAR molecule is a bispecific CAR molecule having a binding specificity, e.g., a first and/or second binding specificity, to BCMA, CD19, CD20, and/or CD22. In one embodiment, the binding specificity is configured with its VL (VL₁) upstream of its VH (VH₁) and the downstream antibody or antibody fragment or antigen binding domains (e.g., scFv) is arranged with its VL (VL₂) upstream of its VH (VH₂), such that the overall bispecific CAR molecule has the arrangement VL₁-VH₁-VL₂-VH₂, from an N- to C-terminal orientation. In some embodiments, the first and/or second binding specificity, to BCMA, CD19, CD20, and/or CD22 (e.g., first and/or second scFv to BCMA, CD19, CD20, and/or CD22) comprises a linker between the VH and the VL regions. In some embodiments, the linker between the VH and the VL regions is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In some embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the VH and VL regions are connected without a linker.

In another embodiment, the binding specificity, e.g., a first and/or second binding specificity, to BCMA, CD19, CD20, and/or CD22 is configured with its VL (VL₁) upstream of its VH (VH₁) and the downstream antibody or antibody fragment or antigen binding domains (e.g., scFv) is arranged with its VH (VH₂) upstream of its VL (VL₂), such that the overall bispecific CAR molecule has the arrangement VL₁-VH₁-VH₂-VL₂, from an N- to C-terminal orientation. In some embodiments, the first and/or second binding specificity, to BCMA, CD19, CD20, and/or CD22 (e.g., first and/or second scFv to BCMA, CD19, CD20, and/or CD22) comprises a linker between the VH and the VL regions. In some embodiments, the linker between the VH and the VL regions is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In some embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the VH and VL regions are connected without a linker.

In another embodiment, the binding specificity, e.g., a first and/or second binding specificity, to BCMA, CD19, CD20, and/or CD22 is configured with its VH (VH₁) upstream of its VL (VL₁) and the downstream antibody or antibody fragment or antigen binding domain (e.g., scFv) is arranged with its VL (VL₂) upstream of its VH (VH₂), such that the overall bispecific CAR molecule has the arrangement VH₁-VL₁-VL₂-VH₂, from an N- to C-terminal orientation. In some embodiments, the first and/or second binding specificity, to BCMA, CD19, CD20, and/or CD22 (e.g., first and/or second scFv to BCMA, CD19, CD20, and/or CD22) comprises a linker between the VH and the VL regions. In some embodiments, the linker between the VH and the VL regions is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In some embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the VH and VL regions are connected without a linker.

In another embodiment, the binding specificity, e.g., a first and/or second binding specificity, to BCMA, CD19, CD20, and/or CD22 is configured with its VH (VH₁) upstream of its VL (VL₁) and the downstream antibody or antibody fragment or antigen binding domain (e.g., scFv) is arranged with its VH (VH₂) upstream of its VL (VL₂), such that the overall bispecific CAR molecule has the arrangement VH₁-VL₁-VH₂-VL₂, from an N- to C-terminal orientation. In some embodiments, the first and/or second binding specificity, to BCMA, CD19, CD20, and/or CD22 (e.g., first and/or second scFv to BCMA, CD19, CD20, and/or CD22) comprises a linker between the VH and the VL regions. In some embodiments, the linker between the VH and the VL regions is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In some embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the VH and VL regions are connected without a linker.

In some embodiments, the bispecific CAR molecule comprises a first binding specificity to BCMA, e.g., any of the binding specificities to BCMA described herein, and a second binding specificity to CD19, e.g., any of the binding specificities to CD19 as described herein. In some embodiments, the bispecific CAR molecule comprises a first binding specificity to BCMA, e.g., any of the binding specificities to BCMA described herein, and a second binding specificity to CD20, e.g., any of the binding specificities to CD20 as described herein. In some embodiments, the bispecific CAR molecule comprises a first binding specificity to BCMA, e.g., any of the binding specificities to BCMA described herein, and a second binding specificity to CD22, e.g., any of the binding specificities to CD22 as described herein. In one embodiment, the first and second binding specificity are in a contiguous polypeptide chain, e.g., a single chain. In some embodiments, the first and second binding specificities, optionally, comprise a linker as described herein. In some embodiments, the linker is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In some embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the linker comprises, e.g., consists of, the amino acid sequence: LAEAAAK (SEQ ID NO: 2033).

In some embodiments, the CAR molecule disclosed herein comprises a bispecific CAR comprising a first and second binding specificities, e.g., as described herein (e.g., two antibody molecules, e.g., two scFvs as described herein). In some embodiments, the bispecific CAR comprises two antibody molecules, wherein the first binding specificity, e.g., the first antibody molecule (e.g., the first antigen binding domain, e.g., the first scFv) is closer to the transmembrane domain, also referred to herein as the proximal antibody molecule (e.g., proximal antigen binding domain) and the second binding specificity, e.g., the second antibody molecule (e.g., second antigen binding domain, e.g., the second scFv) is further away from the membrane, also referred to herein as the distal antibody molecule (e.g., the distal antigen binding domain). Thus, from N-to-C-terminus, the CAR molecule comprises a distal binding specificity, e.g., a distal antibody molecule (e.g., a distal antigen binding domain, e.g., a distal scFV or scFv2), optionally, a linker, followed by a proximal binding specificity, e.g., a proximal antibody molecule (e.g., a proximal antigen binding domain, e.g., a proximal scFv or scFv1), optionally via a linker, to a transmembrane domain and an intracellular domain, e.g., as described herein. In some embodiments, the CAR molecule comprises a proximal or distal binding specificity for BCMA, e.g., a BCMA binding specificity as described herein. In one embodiment, the CAR molecule comprises a proximal binding specificity for BCMA, e.g., a BCMA binding specificity as described herein, and a distal binding specificity for CD19, e.g., a CD19 binding specificity as described herein. In one embodiment, the CAR molecule comprises a proximal binding specificity for BCMA, e.g., a BCMA binding specificity as described herein, and a distal binding specificity for CD20, e.g., a CD20 binding specificity as described herein. In one embodiment, the CAR molecule comprises a proximal binding specificity for BCMA, e.g., a BCMA binding specificity as described herein, and a distal binding specificity for CD22, e.g., a CD22 binding specificity as described herein. In one embodiment, the CAR molecule comprises a distal binding specificity for BCMA, e.g., a BCMA binding specificity as described herein, and a proximal binding specificity for CD19, e.g., a CD19 binding specificity as described herein. In one embodiment, the CAR molecule comprises a distal binding specificity for BCMA, e.g., a BCMA binding specificity as described herein, and a proximal binding specificity for CD20, e.g., a CD20 binding specificity as described herein. In one embodiment, the CAR molecule comprises a distal binding specificity for BCMA, e.g., a BCMA binding specificity as described herein, and a proximal binding specificity for CD22, e.g., a CD22 binding specificity as described herein.

In one embodiment, the CAR molecule comprises a distal to the membrane binding specificity to BCMA, e.g., a VL1-VH1 binding specificity to BCMA, and a proximal to the membrane binding specificity to CD19, CD20, or CD22, e.g., a VL2-VH2 or VH2-VL1 binding specificity to CD19. In one embodiment, the first and second binding specificity are in a contiguous polypeptide chain, e.g., a single chain. In some embodiments, the first and second binding specificities, optionally, comprise a linker as described herein. In some embodiments, the linker is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In some embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the linker comprises, e.g., consists of, the amino acid sequence: LAEAAAK (SEQ ID NO: 2033).

In one embodiment, the CAR molecule comprises a proximal to the membrane binding specificity to BCMA, e.g., a VL1-VH1 binding specificity to BCMA, and a distal to the membrane binding specificity to CD19, CD20, or CD22, e.g., a VL2-VH2 or VH2-VL1 binding specificity to CD19, CD20, or CD22. In one embodiment, the first and second binding specificity are in a contiguous polypeptide chain, e.g., a single chain. In some embodiments, the first and second binding specificities, optionally, comprise a linker as described herein. In some embodiments, the linker is a (Gly₄-Ser)ₙ linker (SEQ ID NO: 2034), wherein n is 1, 2, 3, 4, 5, or 6. In some embodiments, the linker is (Gly₄-Ser)ₙ, wherein n=1 (SEQ ID NO: 1032), e.g., the linker has the amino acid sequence Gly₄-Ser (SEQ ID NO: 1032). In some embodiments, the linker is (Gly₄-Ser)ₙ, wherein n=3 (SEQ ID NO: 1040). In some embodiments, the linker is (Gly₄-Ser)ₙ, wherein n=4 (SEQ ID NO: 1039). In some embodiments, the linker comprises, e.g., consists of, the amino acid sequence: LAEAAAK (SEQ ID NO: 2033).

### FCRL2 or FCRL5 Inhibitor

In some embodiments, the BCMA CAR-expressing cell therapy is administered in combination with a FCRL2 or FCRL5 inhibitor. In some embodiments, the FCRL2 or FCRL5 inhibitor is an anti-FCRL2 antibody molecule, e.g., a bispecific antibody molecule, e.g., a bispecific antibody that binds to FCRL2 and CD3. In some embodiments, the FCRL2 or FCRL5 inhibitor is an anti-FCRL5 antibody molecule, e.g., a bispecific antibody molecule, e.g., a bispecific antibody that binds to FCRL5 and CD3. In some embodiments, the FCRL2 or FCRL5 inhibitor is a FCRL2 CAR-expressing cell therapy. In some embodiments, the FCRL2 or FCRL5 inhibitor is a FCRL5 CAR-expressing cell therapy.

Exemplary anti-FCRL5 antibody molecules are disclosed in US20150098900, US20160368985, WO2017096120 (e.g., antibodies ET200-001, ET200-002, ET200-003, ET200-006, ET200-007, ET200-008, ET200-009, ET200-010, ET200-011, ET200-012, ET200-013, ET200-014, ET200-015, ET200-016, ET200-017, ET200-018, ET200-019, ET200-020, ET200-021, ET200-022, ET200-023, ET200-024, ET200-025, ET200-026, ET200-027, ET200-028, ET200-029, ET200-030, ET200-031, ET200-032, ET200-033, ET200-034, ET200-035, ET200-037, ET200-038, ET200-039, ET200-040, ET200-041, ET200-042, ET200-043, ET200-044, ET200-045, ET200-069, ET200-078, ET200-079, ET200-081, ET200-097, ET200-098, ET200-099, ET200-100, ET200-101, ET200-102, ET200-103, ET200-104, ET200-105, ET200-106, ET200-107, ET200-108, ET200-109, ET200-110, ET200-111, ET200-112, ET200-113, ET200-114, ET200-115, ET200-116, ET200-117, ET200-118, ET200-119, ET200-120, ET200-121, ET200-122, ET200-123, ET200-125, ET200-005 and ET200-124 disclosed in WO2017096120), herein incorporated by reference in their entirety.

Exemplary FCRL5 CAR molecules are disclosed in WO2016090337, herein incorporated by reference in its entirety.

IL-15 and/or IL-15Ra

In some embodiments, the BCMA CAR-expressing cell therapy is administered in combination with IL-15. In some embodiments, the BCMA CAR-expressing cell therapy is administered in combination with an IL15/IL-15Ra complex. In some embodiments, the IL-15/IL-15Ra complex is chosen from NIZ985 (Novartis), ATL-803 (Altor) or CYP0150 (Cytune).

### Exemplary IL-15/IL-15Ra Complexes

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 1001 in Table 16 and the soluble form of human IL-15Ra comprises an amino acid sequence of SEQ ID NO:1002 in Table 16, as described in WO 2014/066527, incorporated by reference in its entirety. The molecules described herein can be made by vectors, host cells, and methods described in WO 2007/084342, incorporated by reference in its entirety.

TABLE 16
Amino acid and nucleotide sequences of exemplary IL-15/IL-15Ra
complexes
NIZ985

| | | |
|---|---|---|
| SEQ ID NO: | Human IL-15 | NWVNVISDLKKIEDLIQSMHIDATLYTESDVHPSCKVTAM |
| 1001 | | KCFLLELQVISLESGDASIHDTVENLIILANNSLSSNGNVTE |
| | | SGCKECEELEEKNIKEFLQSFVHIVQMFINTS |
| SEQ ID NO: | Human | ITCPPPMSVEHADIWVKSYSLYSRERYICNSGFKRKAGTSS |
| 1002 | Soluble IL- | LTECVLNKATNVAHWTTPSLKCIRDPALVHQRPAPPSTVT |
| | 15Ra | TAGVTPQPESLSPSGKEPAASSPSSNNTAATTAAIVPGSQL |
| | | MPSKSPSTGTTEISSHESSHGTPSQTTAKNWELTASASHQP |
| | | PGVYPQG |

### Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table 17.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table 17.

TABLE 17
Amino acid sequences of other exemplary IL-15/IL-15Ra complexes
ALT-803 (Altor)

| | | |
|---|---|---|
| SEQ ID NO: | IL-15N72D | NWVNVISDLKKIEDLIQSMHIDATLYTESDVHPSCKVTAMK |
| 1003 | | CFLLELQVISLESGDASIHDTVENLIILANDSLSSNGNVTESG |
| | | CKECEELEEKNIKEFLQSFVHIVQMFINTS |
| SEQ ID NO: | IL-15RaSu/Fc | ITCPPPMSVEHADIWVKSYSLYSRERYICNSGFKRKAGTSSL |
| 1004 | | TECVLNKATNVAHWTTPSLKCIREPKSCDKTHTCPPCPAPE |
| | | LLGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | RDELTKNQVSLTCLVKGFYPSDIAVEWESNGQPENNYKTTP |
| | | PVLDSDGSFFLYSKLTVDKSRWQQGNVFSCSVMHEALHNH |
| | | YTQKSLSLSPGK |

IL-15/IL-15Ra sushi domain fusion (Cytune)

| | | |
|---|---|---|
| SEQ ID | Human IL-15 | NWVNVISDLKKIEDLIQSMHIDATLYTESDVHPSCKVTAMK |
| NO: 1005 | | CFLLELQVISLESGDASIHDTVENLIILANNSLSSNGNVTESG |
| | | CKECEELEXKNIKEFLQSFVHIVQMFINTS |
| | | Where X is E or K |
| SEQ ID | Human IL- | ITCPPPMSVEHADIWVKSYSLYSRERYICNSGFKRKAGTSSL |
| NO: 1006 | 15Ra sushi | TECVLNKATNVAHWTTPSLKCIRDPALVHQRPAPP |
| | and hinge | |
| | domains | |

### PD-1 Inhibitor

In some embodiments, the BCMA CAR-expressing cell therapy is administered in combination with a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 18 (e.g., from the heavy and light chain variable region sequences of BAP049-Clone-E or BAP049-Clone-B disclosed in Table 18), or encoded by a nucleotide sequence shown in Table 18. In some embodiments, the CDRs are according to the Kabat definition (e.g., as set out in Table 18). In some embodiments, the CDRs are according to the Chothia definition (e.g., as set out in Table 18). In some embodiments, the CDRs are according to the combined CDR definitions of both Kabat and Chothia (e.g., as set out in Table 18). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 18, or encoded by a nucleotide sequence shown in Table 18.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 18.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 18.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 18
Amino acid and nucleotide sequences of
exemplary anti-PD-1 antibody molecules

| | |
|---|---|
| BAP049-Clone-B HC | |
| SEQ ID NO: 501 (Kabat) | HCDR1 |
| SEQ ID NO: 502 (Kabat) | HCDR2 |
| SEQ ID NO: 503 (Kabat) | HCDR3 |
| SEQ ID NO: 504 (Chothia) | HCDR1 |
| SEQ ID NO: 505 (Chothia) | HCDR2 |
| SEQ ID NO: 503 (Chothia) | HCDR3 |
| SEQ ID NO: 506 | VH |
| SEQ ID NO: 507 | DNA VH |
| SEQ ID NO: 508 | Heavy chain |
| SEQ ID NO: 509 | DNA heavy chain |
| BAP049-Clone-B LC | |
| SEQ ID NO: 510 (Kabat) | LCDR1 |
| SEQ ID NO: 511 (Kabat) | LCDR2 |
| SEQ ID NO: 512 (Kabat) | LCDR3 |
| SEQ ID NO: 513 (Chothia) | LCDR1 |
| SEQ ID NO: 514 (Chothia) | LCDR2 |
| SEQ ID NO: 515 (Chothia) | LCDR3 |
| SEQ ID NO: 516 | VL |
| SEQ ID NO: 517 | DNA VL |
| SEQ ID NO: 518 | Light chain |
| SEQ ID NO: 519 | DNA light chain |
| BAP049-Clone-E HC | |
| SEQ ID NO: 501 (Kabat) | HCDR1 |
| SEQ ID NO: 502 (Kabat) | HCDR2 |
| SEQ ID NO: 503 (Kabat) | HCDR3 |
| SEQ ID NO: 504 (Chothia) | HCDR1 |
| SEQ ID NO: 505 (Chothia) | HCDR2 |
| SEQ ID NO: 503 (Chothia) | HCDR3 |
| SEQ ID NO: 506 | VH |
| SEQ ID NO: 507 | DNA VH |
| SEQ ID NO: 508 | Heavy chain |
| SEQ ID NO: 509 | DNA heavy chain |
| BAP049-Clone-E LC | |
| SEQ ID NO: 510 (Kabat) | LCDR1 |
| SEQ ID NO: 511 (Kabat) | LCDR2 |
| SEQ ID NO: 512 (Kabat) | LCDR3 |
| SEQ ID NO: 513 (Chothia) | LCDR1 |
| SEQ ID NO: 514 (Chothia) | LCDR2 |
| SEQ ID NO: 515 (Chothia) | LCDR3 |
| SEQ ID NO: 520 | VL |
| SEQ ID NO: 521 | DNA VL |
| SEQ ID NO: 522 | Light chain |
| SEQ ID NO: 523 | DNA light chain |
| BAP049-Clone-B HC | |
| SEQ ID NO: 524 (Kabat) | HCDR1 |
| SEQ ID NO: 525 (Kabat) | HCDR2 |
| SEQ ID NO: 526 (Kabat) | HCDR3 |
| SEQ ID NO: 527 (Chothia) | HCDR1 |
| SEQ ID NO: 528 (Chothia) | HCDR2 |
| SEQ ID NO: 526 (Chothia) | HCDR3 |
| BAP049-Clone-B LC | |
| SEQ ID NO: 529 (Kabat) | LCDR1 |
| SEQ ID NO: 530 (Kabat) | LCDR2 |
| SEQ ID NO: 531 (Kabat) | LCDR3 |
| SEQ ID NO: 532 (Chothia) | LCDR1 |
| SEQ ID NO: 533 (Chothia) | LCDR2 |
| SEQ ID NO: 534 (Chothia) | LCDR3 |
| BAP049-Clone-E HC | |
| SEQ ID NO: 524 (Kabat) | HCDR1 |
| SEQ ID NO: 525 (Kabat) | HCDR2 |
| SEQ ID NO: 526 (Kabat) | HCDR3 |
| SEQ ID NO: 527 (Chothia) | HCDR1 |
| SEQ ID NO: 528 (Chothia) | HCDR2 |
| SEQ ID NO: 526 (Chothia) | HCDR3 |
| BAP049-Clone-E LC | |
| SEQ ID NO: 529 (Kabat) | LCDR1 |
| SEQ ID NO: 530 (Kabat) | LCDR2 |
| SEQ ID NO: 531 (Kabat) | LCDR3 |
| SEQ ID NO: 532 (Chothia) | LCDR1 |
| SEQ ID NO: 533 (Chothia) | LCDR2 |
| SEQ ID NO: 534 (Chothia) | LCDR3 |

### Other Exemplary PD-1 Inhibitors

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 19.

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 19.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 19.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 19
Amino acid sequences of other exemplary
anti-PD-1 antibody molecules

| | |
|---|---|
| Nivolumab | |
| SEQ ID NO: 535 | Heavy chain |
| SEQ ID NO: 536 | Light chain |
| Pembrolizumab | |
| SEQ ID NO: 537 | Heavy chain |
| SEQ ID NO: 538 | Light chain |
| Pidilizumab | |
| SEQ ID NO: 539 | Heavy chain |
| SEQ ID NO: 540 | Light chain |

### PD-L1 Inhibitors

In some embodiments, the BCMA CAR-expressing cell therapy is administered in combination with a PD-L1 inhibitor. In some embodiments, the PD-L1 inhibitor is chosen from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

### Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 20 (e.g., from the heavy and light chain variable region sequences of BAP058-Clone 0 or BAP058-Clone N disclosed in Table 20), or encoded by a nucleotide sequence shown in Table 20. In some embodiments, the CDRs are according to the Kabat definition (e.g., as set out in Table 20). In some embodiments, the CDRs are according to the Chothia definition (e.g., as set out in Table 20). In some embodiments, the CDRs are according to the combined CDR definitions of both Kabat and Chothia (e.g., as set out in Table 20). In one embodiment, the combination of Kabat and Chothia CDR of VH CDR1 comprises the amino acid sequence GYTFTSYWMY (SEQ ID NO: 647). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 20, or encoded by a nucleotide sequence shown in Table 20.

In one embodiment, the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611, each disclosed in Table 20.

In one embodiment, the anti-PD-L1 antibody molecule comprises a VH comprising a VHCDR1 encoded by the nucleotide sequence of SEQ ID NO: 628, a VHCDR2 encoded by the nucleotide sequence of SEQ ID NO: 629, and a VHCDR3 encoded by the nucleotide sequence of SEQ ID NO: 630; and a VL comprising a VLCDR1 encoded by the nucleotide sequence of SEQ ID NO: 633, a VLCDR2 encoded by the nucleotide sequence of SEQ ID NO: 634, and a VLCDR3 encoded by the nucleotide sequence of SEQ ID NO: 635, each disclosed in Table 20.

In one embodiment, the anti-PD-L1 antibody molecule comprises a VH comprising the amino acid sequence of SEQ ID NO: 606, or an amino acid sequence at least 85%, 90%, 95%, or 99% identical or higher to SEQ ID NO: 606. In one embodiment, the anti-PD-L1 antibody molecule comprises a VL comprising the amino acid sequence of SEQ ID NO: 616, or an amino acid sequence at least 85%, 90%, 95%, or 99% identical or higher to SEQ ID NO: 616. In one embodiment, the anti-PD-L1 antibody molecule comprises a VH comprising the amino acid sequence of SEQ ID NO: 620, or an amino acid sequence at least 85%, 90%, 95%, or 99% identical or higher to SEQ ID NO: 620. In one embodiment, the anti-PD-L1 antibody molecule comprises a VL comprising the amino acid sequence of SEQ ID NO: 624, or an amino acid sequence at least 85%, 90%, 95%, or 99% identical or higher to SEQ ID NO: 624. In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

In one embodiment, the antibody molecule comprises a VH encoded by the nucleotide sequence of SEQ ID NO: 607, or a nucleotide sequence at least 85%, 90%, 95%, or 99% identical or higher to SEQ ID NO: 607. In one embodiment, the antibody molecule comprises a VL encoded by the nucleotide sequence of SEQ ID NO: 617, or a nucleotide sequence at least 85%, 90%, 95%, or 99% identical or higher to SEQ ID NO: 617. In one embodiment, the antibody molecule comprises a VH encoded by the nucleotide sequence of SEQ ID NO: 621, or a nucleotide sequence at least 85%, 90%, 95%, or 99% identical or higher to SEQ ID NO: 621. In one embodiment, the antibody molecule comprises a VL encoded by the nucleotide sequence of SEQ ID NO: 625, or a nucleotide sequence at least 85%, 90%, 95%, or 99% identical or higher to SEQ ID NO: 625. In one embodiment, the antibody molecule comprises a VH encoded by the nucleotide sequence of SEQ ID NO: 607 and a VL encoded by the nucleotide sequence of SEQ ID NO: 617. In one embodiment, the antibody molecule comprises a VH encoded by the nucleotide sequence of SEQ ID NO: 621 and a VL encoded by the nucleotide sequence of SEQ ID NO: 625.

In one embodiment, the anti-PD-L1 antibody molecule comprises a heavy chain comprising the amino acid sequence of SEQ ID NO: 608, or an amino acid sequence at least 85%, 90%, 95%, or 99% identical or higher to SEQ ID NO: 608. In one embodiment, the anti-PD-L1 antibody molecule comprises a light chain comprising the amino acid sequence of SEQ ID NO: 618, or an amino acid sequence at least 85%, 90%, 95%, or 99% identical or higher to SEQ ID NO: 618. In one embodiment, the anti-PD-L1 antibody molecule comprises a heavy chain comprising the amino acid sequence of SEQ ID NO: 622, or an amino acid sequence at least 85%, 90%, 95%, or 99% identical or higher to SEQ ID NO: 622. In one embodiment, the anti-PD-L1 antibody molecule comprises a light chain comprising the amino acid sequence of SEQ ID NO: 626, or an amino acid sequence at least 85%, 90%, 95%, or 99% identical or higher to SEQ ID NO: 626. In one embodiment, the anti-PD-L1 antibody molecule comprises a heavy chain comprising the amino acid sequence of SEQ ID NO: 608 and a light chain comprising the amino acid sequence of SEQ ID NO: 618. In one embodiment, the anti-PD-L1 antibody molecule comprises a heavy chain comprising the amino acid sequence of SEQ ID NO: 622 and a light chain comprising the amino acid sequence of SEQ ID NO: 626.

In one embodiment, the antibody molecule comprises a heavy chain encoded by the nucleotide sequence of SEQ ID NO: 615, or a nucleotide sequence at least 85%, 90%, 95%, or 99% identical or higher to SEQ ID NO: 615. In one embodiment, the antibody molecule comprises a light chain encoded by the nucleotide sequence of SEQ ID NO: 619, or a nucleotide sequence at least 85%, 90%, 95%, or 99% identical or higher to SEQ ID NO: 619. In one embodiment, the antibody molecule comprises a heavy chain encoded by the nucleotide sequence of SEQ ID NO: 623, or a nucleotide sequence at least 85%, 90%, 95%, or 99% identical or higher to SEQ ID NO: 623. In one embodiment, the antibody molecule comprises a light chain encoded by the nucleotide sequence of SEQ ID NO: 627, or a nucleotide sequence at least 85%, 90%, 95%, or 99% identical or higher to SEQ ID NO: 627. In one embodiment, the antibody molecule comprises a heavy chain encoded by the nucleotide sequence of SEQ ID NO: 615 and a light chain encoded by the nucleotide sequence of SEQ ID NO: 619. In one embodiment, the antibody molecule comprises a heavy chain encoded by the nucleotide sequence of SEQ ID NO: 623 and a light chain encoded by the nucleotide sequence of SEQ ID NO: 627. The antibody molecules described herein can be made by vectors, host cells, and methods described in US 2016/0108123, incorporated by reference in its entirety.

TABLE 20
Amino acid and nucleotide sequences of
exemplary anti-PD-L1 antibody molecules

| | |
|---|---|
| BAP058-Clone O HC | |
| SEQ ID NO: 601 (Kabat) | HCDR1 |
| SEQ ID NO: 602 (Kabat) | HCDR2 |
| SEQ ID NO: 603 (Kabat) | HCDR3 |
| SEQ ID NO: 604 (Chothia) | HCDR1 |
| SEQ ID NO: 605 (Chothia) | HCDR2 |
| SEQ ID NO: 603 (Chothia) | HCDR3 |
| SEQ ID NO: 606 | VH |
| SEQ ID NO: 607 | DNA VH |
| SEQ ID NO: 608 | Heavy chain |
| SEQ ID NO: 615 | DNA heavy chain |
| BAP058-Clone O LC | |
| SEQ ID NO: 609 (Kabat) | LCDR1 |
| SEQ ID NO: 610 (Kabat) | LCDR2 |
| SEQ ID NO: 611 (Kabat) | LCDR3 |
| SEQ ID NO: 612 (Chothia) | LCDR1 |
| SEQ ID NO: 613 (Chothia) | LCDR2 |
| SEQ ID NO: 614 (Chothia) | LCDR3 |
| SEQ ID NO: 616 | VL |
| SEQ ID NO: 617 | DNA VL |
| SEQ ID NO: 618 | Light chain |
| SEQ ID NO: 619 | DNA light chain |
| BAP058-Clone N HC | |
| SEQ ID NO: 601 (Kabat) | HCDR1 |
| SEQ ID NO: 602 (Kabat) | HCDR2 |
| SEQ ID NO: 603 (Kabat) | HCDR3 |
| SEQ ID NO: 604 (Chothia) | HCDR1 |
| SEQ ID NO: 605 (Chothia) | HCDR2 |
| SEQ ID NO: 603 (Chothia) | HCDR3 |
| SEQ ID NO: 620 | VH |
| SEQ ID NO: 621 | DNA VH |
| SEQ ID NO: 622 | Heavy chain |
| SEQ ID NO: 623 | DNA heavy chain |
| BAP058-Clone N LC | |
| SEQ ID NO: 609 (Kabat) | LCDR1 |
| SEQ ID NO: 610 (Kabat) | LCDR2 |
| SEQ ID NO: 611 (Kabat) | LCDR3 |
| SEQ ID NO: 612 (Chothia) | LCDR1 |
| SEQ ID NO: 613 (Chothia) | LCDR2 |
| SEQ ID NO: 614 (Chothia) | LCDR3 |
| SEQ ID NO: 624 | VL |
| SEQ ID NO: 625 | DNA VL |
| SEQ ID NO: 626 | Light chain |
| SEQ ID NO: 627 | DNA light chain |
| BAP058-Clone O HC | |
| SEQ ID NO: 628 (Kabat) | HCDR1 |
| SEQ ID NO: 629 (Kabat) | HCDR2 |
| SEQ ID NO: 630 (Kabat) | HCDR3 |
| SEQ ID NO: 631 (Chothia) | HCDR1 |
| SEQ ID NO: 632 (Chothia) | HCDR2 |
| SEQ ID NO: 630 (Chothia) | HCDR3 |
| BAP058-Clone O LC | |
| SEQ ID NO: 633 (Kabat) | LCDR1 |
| SEQ ID NO: 634 (Kabat) | LCDR2 |
| SEQ ID NO: 635 (Kabat) | LCDR3 |
| SEQ ID NO: 636 (Chothia) | LCDR1 |
| SEQ ID NO: 637 (Chothia) | LCDR2 |
| SEQ ID NO: 638 (Chothia) | LCDR3 |
| BAP058-Clone N HC | |
| SEQ ID NO: 628 (Kabat) | HCDR1 |
| SEQ ID NO: 629 (Kabat) | HCDR2 |
| SEQ ID NO: 630 (Kabat) | HCDR3 |
| SEQ ID NO: 631 (Chothia) | HCDR1 |
| SEQ ID NO: 632 (Chothia) | HCDR2 |
| SEQ ID NO: 630 (Chothia) | HCDR3 |
| BAP058-Clone N LC | |
| SEQ ID NO: 633 (Kabat) | LCDR1 |
| SEQ ID NO: 634 (Kabat) | LCDR2 |
| SEQ ID NO: 635 (Kabat) | LCDR3 |
| SEQ ID NO: 636 (Chothia) | LCDR1 |
| SEQ ID NO: 637 (Chothia) | LCDR2 |
| SEQ ID NO: 638 (Chothia) | LCDR3 |

### Other Exemplary PD-L1 Inhibitors

In one embodiment, the anti-PD-L1 antibody molecule is Atezolizumab (Genentech/Roche), also known as MPDL3280A, RG7446, R05541267, YW243.55.S70, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizuma, e.g., as disclosed in Table 21.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 21.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 21.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 21.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 21
Amino acid sequences of other exemplary
anti-PD-L1 antibody molecules

| | |
|---|---|
| Atezolizumab | |
| SEQ ID NO: 639 | Heavy chain |
| SEQ ID NO: 640 | Light chain |
| Avelumab | |
| SEQ ID NO: 641 | Heavy chain |
| SEQ ID NO: 642 | Light chain |
| Durvalumab | |
| SEQ ID NO: 643 | Heavy chain |
| SEQ ID NO: 644 | Light chain |
| BMS-936559 | |
| SEQ ID NO: 645 | VH |
| SEQ ID NO: 646 | VL |

### LAG-3 Inhibitors

In some embodiments, the BCMA CAR-expressing cell therapy is administered in combination with a LAG-3 inhibitor. In some embodiments, the LAG-3 inhibitor is chosen from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 22 (e.g., from the heavy and light chain variable region sequences of BAP050-Clone I or BAP050-Clone J disclosed in Table 22), or encoded by a nucleotide sequence shown in Table 22. In some embodiments, the CDRs are according to the Kabat definition (e.g., as set out in Table 22). In some embodiments, the CDRs are according to the Chothia definition (e.g., as set out in Table 22). In some embodiments, the CDRs are according to the combined CDR definitions of both Kabat and Chothia (e.g., as set out in Table 22). In one embodiment, the combination of Kabat and Chothia CDR of VH CDR1 comprises the amino acid sequence GFTLTNYGMN (SEQ ID NO: 766). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 22, or encoded by a nucleotide sequence shown in Table 22.

In one embodiment, the anti-LAG-3 antibody molecule comprises a heavy chain variable region (VH) comprising a VHCDR1 amino acid sequence of SEQ ID NO: 701, a VHCDR2 amino acid sequence of SEQ ID NO: 702, and a VHCDR3 amino acid sequence of SEQ ID NO: 703; and a light chain variable region (VL) comprising a VLCDR1 amino acid sequence of SEQ ID NO: 710, a VLCDR2 amino acid sequence of SEQ ID NO: 711, and a VLCDR3 amino acid sequence of SEQ ID NO: 712, each disclosed in Table 22.

In one embodiment, the anti-LAG-3 antibody molecule comprises a VH comprising a VHCDR1 encoded by the nucleotide sequence of SEQ ID NO: 736 or 737, a VHCDR2 encoded by the nucleotide sequence of SEQ ID NO: 738 or 739, and a VHCDR3 encoded by the nucleotide sequence of SEQ ID NO: 740 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 22.

In one embodiment, the anti-LAG-3 antibody molecule comprises a VH comprising a VHCDR1 encoded by the nucleotide sequence of SEQ ID NO: 758 or 737, a VHCDR2 encoded by the nucleotide sequence of SEQ ID NO: 759 or 739, and a VHCDR3 encoded by the nucleotide sequence of SEQ ID NO: 760 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 22.

In one embodiment, the anti-LAG-3 antibody molecule comprises a VH comprising the amino acid sequence of SEQ ID NO: 706, or an amino acid sequence at least 85%, 90%, 95%, or 99% identical or higher to SEQ ID NO: 706. In one embodiment, the anti-LAG-3 antibody molecule comprises a VL comprising the amino acid sequence of SEQ ID NO: 718, or an amino acid sequence at least 85%, 90%, 95%, or 99% identical or higher to SEQ ID NO: 718. In one embodiment, the anti-LAG-3 antibody molecule comprises a VH comprising the amino acid sequence of SEQ ID NO: 724, or an amino acid sequence at least 85%, 90%, 95%, or 99% identical or higher to SEQ ID NO: 724. In one embodiment, the anti-LAG-3 antibody molecule comprises a VL comprising the amino acid sequence of SEQ ID NO: 730, or an amino acid sequence at least 85%, 90%, 95%, or 99% identical or higher to SEQ ID NO: 730. In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

In one embodiment, the antibody molecule comprises a VH encoded by the nucleotide sequence of SEQ ID NO: 707 or 708, or a nucleotide sequence at least 85%, 90%, 95%, or 99% identical or higher to SEQ ID NO: 707 or 708. In one embodiment, the antibody molecule comprises a VL encoded by the nucleotide sequence of SEQ ID NO: 719 or 720, or a nucleotide sequence at least 85%, 90%, 95%, or 99% identical or higher to SEQ ID NO: 719 or 720. In one embodiment, the antibody molecule comprises a VH encoded by the nucleotide sequence of SEQ ID NO: 725 or 726, or a nucleotide sequence at least 85%, 90%, 95%, or 99% identical or higher to SEQ ID NO: 725 or 726. In one embodiment, the antibody molecule comprises a VL encoded by the nucleotide sequence of SEQ ID NO: 731 or 732, or a nucleotide sequence at least 85%, 90%, 95%, or 99% identical or higher to SEQ ID NO: 731 or 732. In one embodiment, the antibody molecule comprises a VH encoded by the nucleotide sequence of SEQ ID NO: 707 or 708 and a VL encoded by the nucleotide sequence of SEQ ID NO: 719 or 720. In one embodiment, the antibody molecule comprises a VH encoded by the nucleotide sequence of SEQ ID NO: 725 or 726 and a VL encoded by the nucleotide sequence of SEQ ID NO: 731 or 732.

In one embodiment, the anti-LAG-3 antibody molecule comprises a heavy chain comprising the amino acid sequence of SEQ ID NO: 709, or an amino acid sequence at least 85%, 90%, 95%, or 99% identical or higher to SEQ ID NO: 709. In one embodiment, the anti-LAG-3 antibody molecule comprises a light chain comprising the amino acid sequence of SEQ ID NO: 721, or an amino acid sequence at least 85%, 90%, 95%, or 99% identical or higher to SEQ ID NO: 721. In one embodiment, the anti-LAG-3 antibody molecule comprises a heavy chain comprising the amino acid sequence of SEQ ID NO: 727, or an amino acid sequence at least 85%, 90%, 95%, or 99% identical or higher to SEQ ID NO: 727. In one embodiment, the anti-LAG-3 antibody molecule comprises a light chain comprising the amino acid sequence of SEQ ID NO: 733, or an amino acid sequence at least 85%, 90%, 95%, or 99% identical or higher to SEQ ID NO: 733. In one embodiment, the anti-LAG-3 antibody molecule comprises a heavy chain comprising the amino acid sequence of SEQ ID NO: 709 and a light chain comprising the amino acid sequence of SEQ ID NO: 721. In one embodiment, the anti-LAG-3 antibody molecule comprises a heavy chain comprising the amino acid sequence of SEQ ID NO: 727 and a light chain comprising the amino acid sequence of SEQ ID NO: 733.

In one embodiment, the antibody molecule comprises a heavy chain encoded by the nucleotide sequence of SEQ ID NO: 716 or 717, or a nucleotide sequence at least 85%, 90%, 95%, or 99% identical or higher to SEQ ID NO: 716 or 717. In one embodiment, the antibody molecule comprises a light chain encoded by the nucleotide sequence of SEQ ID NO: 722 or 723, or a nucleotide sequence at least 85%, 90%, 95%, or 99% identical or higher to SEQ ID NO: 722 or 723. In one embodiment, the antibody molecule comprises a heavy chain encoded by the nucleotide sequence of SEQ ID NO: 728 or 729, or a nucleotide sequence at least 85%, 90%, 95%, or 99% identical or higher to SEQ ID NO: 728 or 729. In one embodiment, the antibody molecule comprises a light chain encoded by the nucleotide sequence of SEQ ID NO: 734 or 735, or a nucleotide sequence at least 85%, 90%, 95%, or 99% identical or higher to SEQ ID NO: 734 or 735. In one embodiment, the antibody molecule comprises a heavy chain encoded by the nucleotide sequence of SEQ ID NO: 716 or 717 and a light chain encoded by the nucleotide sequence of SEQ ID NO: 722 or 723. In one embodiment, the antibody molecule comprises a heavy chain encoded by the nucleotide sequence of SEQ ID NO: 728 or 729 and a light chain encoded by the nucleotide sequence of SEQ ID NO: 734 or 735.

The antibody molecules described herein can be made by vectors, host cells, and methods described in US 2015/0259420, incorporated by reference in its entirety.

TABLE 22
Amino acid and nucleotide sequences of
exemplary anti-LAG-3 antibody molecules

| | |
|---|---|
| BAP050-Clone I HC | |
| SEQ ID NO: 701 (Kabat) | HCDR1 |
| SEQ ID NO: 702 (Kabat) | HCDR2 |
| SEQ ID NO: 703 (Kabat) | HCDR3 |
| SEQ ID NO: 704 (Chothia) | HCDR1 |
| SEQ ID NO: 705 (Chothia) | HCDR2 |
| SEQ ID NO: 703 (Chothia) | HCDR3 |
| SEQ ID NO: 706 | VH |
| SEQ ID NO: 707 | DNA VH |
| SEQ ID NO: 708 | DNA VH |
| SEQ ID NO: 709 | Heavy chain |
| SEQ ID NO: 716 | DNA heavy chain |
| SEQ ID NO: 717 | DNA heavy chain |
| BAP050-Clone I LC | |
| SEQ ID NO: 710 (Kabat) | LCDR1 |
| SEQ ID NO: 711 (Kabat) | LCDR2 |
| SEQ ID NO: 712 (Kabat) | LCDR3 |
| SEQ ID NO: 713 (Chothia) | LCDR1 |
| SEQ ID NO: 714 (Chothia) | LCDR2 |
| SEQ ID NO: 715 (Chothia) | LCDR3 |
| SEQ ID NO: 718 | VL |
| SEQ ID NO: 719 | DNA VL |
| SEQ ID NO: 720 | DNA VL |
| SEQ ID NO: 721 | Light chain |
| SEQ ID NO: 722 | DNA light chain |
| SEQ ID NO: 723 | DNA light chain |
| BAP050-Clone J HC | |
| SEQ ID NO: 701 (Kabat) | HCDR1 |
| SEQ ID NO: 702 (Kabat) | HCDR2 |
| SEQ ID NO: 703 (Kabat) | HCDR3 |
| SEQ ID NO: 704 (Chothia) | HCDR1 |
| SEQ ID NO: 705 (Chothia) | HCDR2 |
| SEQ ID NO: 703 (Chothia) | HCDR3 |
| SEQ ID NO: 724 | VH |
| SEQ ID NO: 725 | DNA VH |
| SEQ ID NO: 726 | DNA VH |
| SEQ ID NO: 727 | Heavy chain |
| SEQ ID NO: 728 | DNA heavy chain |
| SEQ ID NO: 729 | DNA heavy chain |
| BAP050-Clone J LC | |
| SEQ ID NO: 710 (Kabat) | LCDR1 |
| SEQ ID NO: 711 (Kabat) | LCDR2 |
| SEQ ID NO: 712 (Kabat) | LCDR3 |
| SEQ ID NO: 713 (Chothia) | LCDR1 |
| SEQ ID NO: 714 (Chothia) | LCDR2 |
| SEQ ID NO: 715 (Chothia) | LCDR3 |
| SEQ ID NO: 730 | VL |
| SEQ ID NO: 731 | DNA VL |
| SEQ ID NO: 732 | DNA VL |
| SEQ ID NO: 733 | Light chain |
| SEQ ID NO: 734 | DNA light chain |
| SEQ ID NO: 735 | DNA light chain |
| BAP050-Clone I HC | |
| SEQ ID NO: 736 (Kabat) | HCDR1 |
| SEQ ID NO: 737 (Kabat) | HCDR1 |
| SEQ ID NO: 738 (Kabat) | HCDR2 |
| SEQ ID NO: 739 (Kabat) | HCDR2 |
| SEQ ID NO: 740 (Kabat) | HCDR3 |
| SEQ ID NO: 741 (Kabat) | HCDR3 |
| SEQ ID NO: 742 (Chothia) | HCDR1 |
| SEQ ID NO: 743 (Chothia) | HCDR1 |
| SEQ ID NO: 744 (Chothia) | HCDR2 |
| SEQ ID NO: 745 (Chothia) | HCDR2 |
| SEQ ID NO: 740 (Chothia) | HCDR3 |
| SEQ ID NO: 741 (Chothia) | HCDR3 |
| BAP050-Clone I LC | |
| SEQ ID NO: 746 (Kabat) | LCDR1 |
| SEQ ID NO: 747 (Kabat) | LCDR1 |
| SEQ ID NO: 748 (Kabat) | LCDR2 |
| SEQ ID NO: 749 (Kabat) | LCDR2 |
| SEQ ID NO: 750 (Kabat) | LCDR3 |
| SEQ ID NO: 751 (Kabat) | LCDR3 |
| SEQ ID NO: 752 (Chothia) | LCDR1 |
| SEQ ID NO: 753 (Chothia) | LCDR1 |
| SEQ ID NO: 754 (Chothia) | LCDR2 |
| SEQ ID NO: 755 (Chothia) | LCDR2 |
| SEQ ID NO: 756 (Chothia) | LCDR3 |
| SEQ ID NO: 757 (Chothia) | LCDR3 |
| BAP050-Clone J HC | |
| SEQ ID NO: 758 (Kahat) | HCDR1 |
| SEQ ID NO: 737 (Kahat) | HCDR1 |
| SEQ ID NO: 759 (Kahat) | HCDR2 |
| SEQ ID NO: 739 (Kabat) | HCDR2 |
| SEQ ID NO: 760 (Kabat) | HCDR3 |
| SEQ ID NO: 741 (Kabat) | HCDR3 |
| SEQ ID NO: 761 (Chothia) | HCDR1 |
| SEQ ID NO: 743 (Chothia) | HCDR1 |
| SEQ ID NO: 744 (Chothia) | HCDR2 |
| SEQ ID NO: 745 (Chothia) | HCDR2 |
| SEQ ID NO: 760 (Chothia) | HCDR3 |
| SEQ ID NO: 741 (Chothia) | HCDR3 |
| BAP050-Clone J LC | |
| SEQ ID NO: 746 (Kabat) | LCDR1 |
| SEQ ID NO: 747 (Kabat) | LCDR1 |
| SEQ ID NO: 748 (Kabat) | LCDR2 |
| SEQ ID NO: 749 (Kabat) | LCDR2 |
| SEQ ID NO: 750 (Kabat) | LCDR3 |
| SEQ ID NO: 751 (Kabat) | LCDR3 |
| SEQ ID NO: 752 (Chothia) | LCDR1 |
| SEQ ID NO: 753 (Chothia) | LCDR1 |
| SEQ ID NO: 754 (Chothia) | LCDR2 |
| SEQ ID NO: 755 (Chothia) | LCDR2 |
| SEQ ID NO: 756 (Chothia) | LCDR3 |
| SEQ ID NO: 757 (Chothia) | LCDR3 |

### Other Exemplary LAG-3 Inhibitors

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 23.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 23. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 23
Amino acid sequences of other exemplary
anti-LAG-3 antibody molecules

| | |
|---|---|
| BMS-986016 | |
| SEQ ID NO: 762 | Heavy chain |
| SEQ ID NO: 763 | Light chain |
| IMP731 | |
| SEQ ID NO: 764 | Heavy chain |
| SEQ ID NO: 765 | Light chain |

### TIM-3 Inhibitors

In some embodiments, the BCMA CAR-expressing cell therapy is administered in combination with a TIM-3 inhibitor. In some embodiments, the TIM-3 inhibitor is MGB453 (Novartis) or TSR-022 (Tesaro).

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 24 (e.g., from the heavy and light chain variable region sequences of ABTIM3-hum11 or ABTIM3-hum03 disclosed in Table 24), or encoded by a nucleotide sequence shown in Table 24. In some embodiments, the CDRs are according to the Kabat definition (e.g., as set out in Table 24). In some embodiments, the CDRs are according to the Chothia definition (e.g., as set out in Table 24). In one embodiment, one or more of the CDRs (or collectively all of the CDRs) have one, two, three, four, five, six or more changes, e g, amino acid substitutions (e.g., conservative amino acid substitutions) or deletions, relative to an amino acid sequence shown in Table 24, or encoded by a nucleotide sequence shown in Table 24.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 24. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 24.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823. In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%, or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 24
Amino acid and nucleotide sequences of
exemplary anti-TIM-3 antibody molecules

| | |
|---|---|
| ABTIM3-hum11 | |
| SEQ ID NO: 801 (Kabat) | HCDR1 |
| SEQ ID NO: 802 (Kabat) | HCDR2 |
| SEQ ID NO: 803 (Kabat) | HCDR3 |
| SEQ ID NO: 804 (Chothia) | HCDR1 |
| SEQ ID NO: 805 (Chothia) | HCDR2 |
| SEQ ID NO: 803 (Chothia) | HCDR3 |
| SEQ ID NO: 806 | VH |
| SEQ ID NO: 807 | DNA VH |
| SEQ ID NO: 808 | Heavy chain |
| SEQ ID NO: 809 | DNA heavy chain |
| SEQ ID NO: 810 (Kabat) | LCDR1 |
| SEQ ID NO: 811 (Kabat) | LCDR2 |
| SEQ ID NO: 812 (Kabat) | LCDR3 |
| SEQ ID NO: 813 (Chothia) | LCDR1 |
| SEQ ID NO: 814 (Chothia) | LCDR2 |
| SEQ ID NO: 815 (Chothia) | LCDR3 |
| SEQ ID NO: 816 | VL |
| SEQ ID NO: 817 | DNA VL |
| SEQ ID NO: 818 | Light chain |
| SEQ ID NO: 819 | DNA light chain |
| ABTIM3-hum03 | |
| SEQ ID NO: 801 (Kabat) | HCDR1 |
| SEQ ID NO: 820 (Kabat) | HCDR2 |
| SEQ ID NO: 803 (Kabat) | HCDR3 |
| SEQ ID NO: 804 (Chothia) | HCDR1 |
| SEQ ID NO: 821 (Chothia) | HCDR2 |
| SEQ ID NO: 803 (Chothia) | HCDR3 |
| SEQ ID NO: 822 | VH |
| SEQ ID NO: 823 | DNA VH |
| SEQ ID NO: 824 | Heavy chain |
| SEQ ID NO: 825 | DNA heavy chain |
| SEQ ID NO: 810 (Kabat) | LCDR1 |
| SEQ ID NO: 811 (Kabat) | LCDR2 |
| SEQ ID NO: 812 (Kabat) | LCDR3 |
| SEQ ID NO: 813 (Chothia) | LCDR1 |
| SEQ ID NO: 814 (Chothia) | LCDR2 |
| SEQ ID NO: 815 (Chothia) | LCDR3 |
| SEQ ID NO: 826 | VL |
| SEQ ID NO: 827 | DNA VL |
| SEQ ID NO: 828 | Light chain |
| SEQ ID NO: 829 | DNA light chain |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 25. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 25
Amino acid sequences of other exemplary
anti-TIM-3 antibody molecules

| | |
|---|---|
| APE5137 | |
| SEQ ID NO: 830 | VH |
| SEQ ID NO: 831 | VL |
| APE5121 | |
| SEQ ID NO: 832 | VH |
| SEQ ID NO: 833 | VL |

### TGF-β Inhibitors

In certain embodiments, the BCMA CAR-expressing cell therapy is administered in combination with a transforming growth factor beta (also known as TGF-β, TGFβ, TGFb, or TGF-beta, used interchangeably herein) inhibitor.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

In some embodiments, the TGF-β inhibitor comprises XOMA 089, or a compound disclosed in International Application Publication No. WO 2012/167143, which is incorporated by reference in its entirety.

XOMA 089 is also known as XPA.42.089. XOMA 089 is a fully human monoclonal antibody that specifically binds and neutralizes TGF-beta 1 and 2 ligands.

The heavy chain variable region of XOMA 089 has the amino acid sequence of:

(SEQ ID NO: 1986)
QVQLVQSGAEVKKPGSSVKVSCKASGGTFSSYAISWVRQAPGQGLEWMG
GIIPIFGTANYAQKFQGRVTITADESTSTAYMELSSLRSEDTAVYYCAR
GLWEVRALPSVYWGQGTLVTVSS (disclosed as SEQ ID
NO: 6 in WO 2012/167143).

The light chain variable region of XOMA 089 has the amino acid sequence of:

(SEQ ID NO: 1987)
SYELTQPPSVSVAPGQTARITCGANDIGSKSVHWYQQKAGQAPVLVVSED
IIRPSGIPERISGSNSGNTATLTISRVEAGDEADYYCQVWDRDSDQYVFG
TGTKVTVLG (disclosed as SEQ ID NO: 8 in WO 2012/
167143).

XOMA 089 binds with high affinity to the human TGF-β isoforms. Generally, XOMA 089 binds with high affinity to TGF-β1 and TGF-β2, and to a lesser extent to TGF-β3. In Biacore assays, the K_{D} of XOMA 089 on human TGF-β is 14.6 pM for TGF-β1, 67.3 pM for TGF-β2, and 948 pM for TGF-β3. Given the high affinity binding to all three TGF-β isoforms, in certain embodiments, XOMA 089 is expected to bind to TGF-β1, 2 and 3 at a dose of XOMA 089 as described herein. XOMA 089 cross-reacts with rodent and cynomolgus monkey TGF-β and shows functional activity in vitro and in vivo, making rodent and cynomolgus monkey relevant species for toxicology studies.

In some embodiments, the TGF-β inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of:

(SEQ ID NO: 1988)
QVQLVQSGAEVKKPGSSVKVSCKASGYTFSSNVISWVRQAPGQGLEWMG
GVIPIVDIANYAQRFKGRVTITADESTSTTYMELSSLRSEDTAVYYCAS
TLGLVLDAMDYWGQGTLVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPSCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGK.

The light chain of fresolimumab has the amino acid sequence of:

(SEQ ID NO: 1989)
ETVLTQSPGTLSLSPGERATLSCRASQSLGSSYLAWYQQKPGQAPRLLI
YGASSRAPGIPDRFSGSGSGTDFTLTISRLEPEDFAVYYCQQYADSPIT
FGQGTRLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

Fresolimumab is disclosed, e.g., in International Application Publication No. WO 2006/086469, and U.S. Pat. Nos. 8,383,780 and 8,591,901, which are incorporated by reference in their entirety.

### Anti-CD73 Antibody Molecules

In some embodiments, the BCMA CAR-expressing cell therapy is administered in combination with an anti-CD73 antibody molecule. In one embodiment, an anti-CD73 antibody molecule is a full antibody molecule or an antigen-binding fragment thereof. In some embodiments, the anti-CD73 antibody molecule is chosen from any of the antibody molecules listed in Table 26. In other embodiments, the anti-CD73 antibody molecule comprises a heavy chain variable domain sequence, a light chain variable domain sequence, or both, as disclosed in Table 26. In certain embodiments, the anti-CD73 antibody molecule binds to a CD73 protein and reduces, e.g., inhibits or antagonizes, an activity of CD73, e.g., human CD73.

In one embodiment, the anti-CD73 antibody molecule is an anti-CD73 antibody disclosed in WO2016/075099, herein incorporated by reference in its entirety. In one embodiment, the anti-CD73 antibody molecule is MEDI 9447, e.g., as disclosed in WO2016/075099. Alternative names for MEDI 9447 include clone 10.3 or 73combo3. MEDI 9447 is an IgG1 antibody that inhibits, e.g., antagonizes, an activity of CD73. MEDI 9447 and other anti-CD73 antibody molecules are also disclosed in WO2016/075176 and US2016/0129108, the entire contents of which are herein incorporated by reference in their entirety.

In one embodiment, the anti-CD73 antibody molecule comprises a heavy chain variable domain, a light chain variable domain, or both, of MEDI 9477. The amino acid sequence of the heavy chain variable domain of MEDI 9477 is disclosed as SEQ ID NO: 1990 (see Table 26). The amino acid sequence of the light chain variable domain of MEDI 9477 is disclosed as SEQ ID NO: 1991 (see Table 26).

In one embodiment, the anti-CD73 antibody molecule is an anti-CD73 antibody disclosed in WO2016/081748, herein incorporated by reference in its entirety. In one embodiment, the anti-CD73 antibody molecule is 11F11, e.g., as disclosed in WO2016/081748. 11F11 is an IgG2 antibody that inhibits, e.g., antagonizes, an activity of CD73. Antibodies derived from 11F11, e.g., CD73.4, and CD73.10; clones of 11F11, e.g., 11F11-1 and 11F11-2; and other anti-CD73 antibody molecules are disclosed in WO2016/081748 and U.S. Pat. No. 9,605,080, the entire contents of which are herein incorporated by reference in their entirety.

In one embodiment, the anti-CD73 antibody molecule comprises a heavy chain variable domain, a light chain variable domain, or both, of 11F11-1 or 11F11-2. The amino acid sequence of the heavy chain variable domain of 11F11-1 is disclosed as SEQ ID NO: 1998 (see Table 26). The amino acid sequence of the light chain variable domain of 11F11-1 is disclosed as SEQ ID NO: 1999 (see Table 26). The amino acid sequence of the heavy chain variable domain of 11F11-2 is disclosed as SEQ ID NO: 1994 (see Table 26). The amino acid sequence of the light chain variable domain of 11F11-2 is disclosed as SEQ ID NO: 1995 (see Table 26). In one embodiment, the anti-CD73 antibody molecule comprises a heavy chain, a light chain, or both, of 11F11-1 or 11F11-2. The heavy and light chain amino acid sequences of 11F11-1 are disclosed as SEQ ID NO: 1996 and SEQ ID NO:1997, respectively (see Table 26). The heavy and light chain amino acid sequences of 11F11-2 are disclosed as SEQ ID NO: 1992 and SEQ ID NO:1993, respectively (see Table 26).

In one embodiment, the anti-CD73 antibody molecule is an anti-CD73 antibody disclosed in e.g., U.S. Pat. No. 9,605,080, herein incorporated by reference in its entirety.

In one embodiment, the anti-CD73 antibody molecule is CD73.4, e.g., as disclosed in U.S. Pat. No. 9,605,080. In one embodiment, the anti-CD73 antibody molecule comprises a heavy chain variable domain, a light chain variable domain, or both, of CD73.4. The amino acid sequence of the heavy chain variable domain of CD73.4 is disclosed as SEQ ID NO: 2000 (see Table 26). The amino acid sequence of the light chain variable domain of 11F11-2 is disclosed as SEQ ID NO: 2001 (see Table 26).

In one embodiment, the anti-CD73 antibody molecule is CD73.10, e.g., as disclosed in U.S. Pat. No. 9,605,080. In one embodiment, the anti-CD73 antibody molecule comprises a heavy chain variable domain, a light chain variable domain, or both, of CD73.10. The amino acid sequence of the heavy chain variable domain of CD73.10 is disclosed as SEQ ID NO: 2002 (see Table 26). The amino acid sequence of the light chain variable domain of 11F11-2 is disclosed as SEQ ID NO: 2003 (see Table 26).

In one embodiment, the anti-CD73 antibody molecule is an anti-CD73 antibody disclosed in WO2009/0203538, herein incorporated by reference in its entirety. In one embodiment, the anti-CD73 antibody molecule is 067-213, e.g., as disclosed in WO2009/0203538.

In one embodiment, the anti-CD73 antibody molecule comprises a heavy chain variable domain, a light chain variable domain, or both, of 067-213. The amino acid sequence of the heavy chain variable domain of 067-213 is disclosed as SEQ ID NO: 2004 (see Table 26). The amino acid sequence of the light chain variable domain of 067-213 is disclosed as SEQ ID NO: 2005 (see Table 26).

In one embodiment, the anti-CD73 antibody molecule is an anti-CD73 antibody disclosed in U.S. Pat. No. 9,090,697, herein incorporated by reference in its entirety. In one embodiment, the anti-CD73 antibody molecule is TY/23, e.g., as disclosed in U.S. Pat. No. 9,090,697. In one embodiment, the anti-CD73 antibody molecule comprises a heavy chain variable domain, a light chain variable domain, or both, of TY/23.

In one embodiment, the anti-CD73 antibody molecule is an anti-CD73 antibody disclosed in WO2016/055609, herein incorporated by reference in its entirety. In one embodiment, the anti-CD73 antibody molecule comprises a heavy chain variable domain, a light chain variable domain, or both, of an anti-CD73 antibody disclosed in WO2016/055609.

In one embodiment, the anti-CD73 antibody molecule is an anti-CD73 antibody disclosed in WO2016/146818, herein incorporated by reference in its entirety. In one embodiment, the anti-CD73 antibody molecule comprises a heavy chain variable domain, a light chain variable domain, or both, of an anti-CD73 antibody disclosed in WO2016/146818.

In one embodiment, the anti-CD73 antibody molecule is an anti-CD73 antibody disclosed in WO2004/079013, herein incorporated by reference in its entirety. In one embodiment, the anti-CD73 antibody molecule comprises a heavy chain variable domain, a light chain variable domain, or both, of an anti-CD73 antibody disclosed in WO2004/079013.

In one embodiment, the anti-CD73 antibody molecule is an anti-CD73 antibody disclosed in WO2012/125850, herein incorporated by reference in its entirety. In one embodiment, the anti-CD73 antibody molecule comprises a heavy chain variable domain, a light chain variable domain, or both, of an anti-CD73 antibody disclosed in WO2012/125850.

In one embodiment, the anti-CD73 antibody molecule is an anti-CD73 antibody disclosed in WO2015/004400, herein incorporated by reference in its entirety. In one embodiment, the anti-CD73 antibody molecule comprises a heavy chain variable domain, a light chain variable domain, or both, of an anti-CD73 antibody disclosed in WO2015/004400.

In one embodiment, the anti-CD73 antibody molecule is an anti-CD73 antibody disclosed in WO2007/146968, herein incorporated by reference in its entirety. In one embodiment, the anti-CD73 antibody molecule comprises a heavy chain variable domain, a light chain variable domain, or both, of an anti-CD73 antibody disclosed in WO2007146968.

In one embodiment, the anti-CD73 antibody molecule is an anti-CD73 antibody disclosed in US2007/0042392, herein incorporated by reference in its entirety. In one embodiment, the anti-CD73 antibody molecule comprises a heavy chain variable domain, a light chain variable domain, or both, of an anti-CD73 antibody disclosed in US2007/0042392.

In one embodiment, the anti-CD73 antibody molecule is an anti-CD73 antibody disclosed in US2009/0138977, herein incorporated by reference in its entirety. In one embodiment, the anti-CD73 antibody molecule comprises a heavy chain variable domain, a light chain variable domain, or both, of an anti-CD73 antibody disclosed in US2009/0138977.

In one embodiment, the anti-CD73 antibody molecule is an anti-CD73 antibody disclosed in Flocke et al., Eur J Cell Biol. 1992 June; 58(1):62-70, herein incorporated by reference in its entirety. In one embodiment, the anti-CD73 antibody molecule comprises a heavy chain variable domain, a light chain variable domain, or both, of an anti-CD73 antibody disclosed in Flocke et al., Eur J Cell Biol. 1992 June; 58(1):62-70.

In one embodiment, the anti-CD73 antibody molecule is an anti-CD73 antibody disclosed in Stagg et al., PNAS. 2010 Jan. 107(4): 1547-1552, herein incorporated by reference in its entirety. In some embodiments, the anti-CD73 antibody molecule is TY/23 or TY11.8, as disclosed in Stagg et al. In one embodiment, the anti-CD73 antibody molecule comprises a heavy chain variable domain, a light chain variable domain, or both, of an anti-CD73 antibody disclosed in Stagg et al.

TABLE 26
Sequences of exemplary anti-CD73 antibody molecules

| | |
|---|---|
| W0201/6075099 | |
| MEDI 9447 | |
| SEQ ID NO: 1990 | VH |
| SEQ ID NO: 1991 | VL |
| WO2016/081748 | |
| 11F11-2 | |
| SEQ ID NO: 1992 | HC |
| SEQ ID NO: 1993 | LC |
| SEQ ID NO: 1994 | VH |
| SEQ ID NO: 1995 | VL kappa |
| WO2016/081748 | |
| 11F11-1 | |
| SEQ ID NO: 1996 | HC |
| SEQ ID NO: 1997 | LC |
| SEQ ID NO: 1998 | VH |
| SEQ ID NO: 1999 | VL kappa |
| U.S. Pat. No. 9,605,080 | |
| CD73.4 | |
| SEQ ID NO: 2000 | VH |
| SEQ ID NO: 2001 | VL kappa |
| U.S. Pat. No. 9,605,080 | |
| CD73.10 | |
| SEQ ID NO: 2002 | VH |
| SEQ ID NO: 2003 | VL kappa |
| U.S. Pat. No. 9,388,249 | |
| 067-213 | |
| SEQ ID NO: 2004 | VH |
| SEQ ID NO: 2005 | VL |

The anti-CD73 antibody molecules used in the combination therapies disclosed herein can include any of the VH/VL sequences disclosed in Table 26, or an amino acid sequence substantially identical thereto (e.g., at least 80%, 85%, 90%, 95%, 99% or more identical thereto). Exemplary sequences for CD73 antibodies include:

(i) the VH and VL amino acid sequences for MEDI 9447, SEQ ID NOs: 1990-1991, respectively, or an amino acid sequence substantially identical thereto (e.g., at least 80%, 85%, 90%, 95%, 99% or more identical to SEQ ID NOs: 1990-1991);

(ii) the HC and LC amino acid sequences for 11F11-2, SEQ ID NOs: 1992-1993, respectively, or an amino acid sequence substantially identical thereto (e.g., at least 80%, 85%, 90%, 95%, 99% or more identical to SEQ ID NOs: 1992-1993);

(iii) the VH and VL amino acid sequences for 11F11-2, SEQ ID NOs: 1994-1995, respectively, or an amino acid sequence substantially identical thereto (e.g., at least 80%, 85%, 90%, 95%, 99% or more identical to SEQ ID NOs: 1994-1995);

(iv) the HC and LC amino acid sequences for 11F11-1, SEQ ID NOs: 1996 and 1997, respectively, or an amino acid sequence substantially identical thereto (e.g., at least 80%, 85%, 90%, 95%, 99% or more identical to SEQ ID NOs: 1996 and 1997);

(v) the VH and VL amino acid sequences for 11F11-1, SEQ ID NOs: 1998-1999, respectively, or an amino acid sequence substantially identical thereto (e.g., at least 80%, 85%, 90%, 95%, 99% or more identical to SEQ ID NOs: 1998-1999);

(vi) the VH and VL amino acid sequences for CD73.4, SEQ ID NOs: 2000-2001, respectively, or an amino acid sequence substantially identical thereto (e.g., at least 80%, 85%, 90%, 95%, 99% or more identical to SEQ ID NOs: 2000-2001);

(vii) the VH and VL amino acid sequences for CD73.10, SEQ ID NOs: 2002-2003, respectively, or an amino acid sequence substantially identical thereto (e.g., at least 80%, 85%, 90%, 95%, 99% or more identical to SEQ ID NOs: 2002-2003); or

(viii) the VH and VL amino acid sequences for 067-213, SEQ ID NOs: 2004-2005, respectively, or an amino acid sequence substantially identical thereto (e.g., at least 80%, 85%, 90%, 95%, 99% or more identical to SEQ ID NOs: 2004-2005).

### IL-17 Inhibitor

In some embodiments, the BCMA CAR-expressing cell therapy is administered in combination with an interleukine-17 (IL-17) inhibitor.

In some embodiments, the IL-17 inhibitor is secukinumab (CAS Registry Numbers: 875356-43-7 (heavy chain) and 875356-44-8 (light chain)). Secukinumab is also known as AIN457 and COSENTYX®. Secukinumab is a recombinant human monoclonal IgG1/κ antibody that binds specifically to IL-17A. It is expressed in a recombinant Chinese Hamster Ovary (CHO) cell line.

Secukinumab is described, e.g., in WO 2006/013107, U.S. Pat. Nos. 7,807,155, 8,119,131, 8,617,552, and EP 1776142. The heavy chain variable region of secukinumab has the amino acid sequence of:

(SEQ ID NO: 2006)
EVQLVESGGGLVQPGGSLRLSCAASGFTFSNYWMNWVRQAPGKGLEWVA
AINQDGSEKYYVGSVKGRFTISRDNAKNSLYLQMNSLRVEDTAVYYCVR
DYYDILTDYYIHYWYFDLWGRGTLVTVSS (disclosed as SEQ
ID NO: 8 in WO 2006/013107).

The light chain variable region of secukinumab has the amino acid sequence of:

(SEQ ID NO: 2007)
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPCT
FGQGTRLEIKR (disclosed as SEQ ID NO: 10 in
WO 2006/013107).

The heavy chain CDR1 of secukinumab has the amino acid sequence of NYWMN (SEQ ID NO: 2008) (disclosed as SEQ ID NO: 1 in WO 2006/013107). The heavy chain CDR2 of secukinumab has the amino acid sequence of AINQDGSEKYYVGSVKG (SEQ ID NO: 2009) (disclosed as SEQ ID NO: 2 in WO 2006/013107). The heavy chain CDR3 of secukinumab has the amino acid sequence of DYYDILTDYYIHYWYFDL (SEQ ID NO: 2010) (disclosed as SEQ ID NO: 3 in WO 2006/013107). The light chain CDR1 of secukinumab has the amino acid sequence of RASQSVSSSYLA (SEQ ID NO: 2011) (disclosed as SEQ ID NO: 4 in WO 2006/013107). The light chain CDR2 of secukinumab has the amino acid sequence of GASSRAT (SEQ ID NO: 2012) (disclosed as SEQ ID NO: 5 in WO 2006/013107). The light chain CDR3 of secukinumab has the amino acid sequence of GASSRAT (SEQ ID NO: 2013) (disclosed as SEQ ID NO: 6 in WO 2006/013107).

In some embodiments, the IL-17 inhibitor is CJM112. CJM112 is also known as XAB4. CJM112 is a fully human monoclonal antibody (e.g., of the IgG1/κ isotype) that targets IL-17A.

CJM112 is disclosed, e.g., in WO 2014/122613. The heavy chain of CJM112 has the amino acid sequence of:

(SEQ ID NO: 2014)
EVQLVESGGDLVQPGGSLRLSCAASGFTFSSYWMSWVRQAPGKGLEWVA
NIKQDGSEKYYVDSVKGRFTISRDNAKNSLYLQMNSLRAEDTAVYYCAR
DRGSLYYWGQGTLVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLS
LSPGK (disclosed as SEQ ID NO: 14 in WO 2014/
122613).

The light chain of CJM112 has the amino acid sequence of:

(SEQ ID NO: 2015)
AIQLTQSPSSLSASVGDRVTITCRPSQGINWELAWYQQKPGKAPKLLIY
DASSLEQGVPSRFSGSGSGTDFTLTISSLQPEDFATYYCQQFNSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC (disclosed as SEQ ID NO: 44
in WO 2014/122613).

CJM112 can bind to human, cynomolgus, mouse and rat IL-17A and neutralize the bioactivity of these cytokines in vitro and in vivo. IL-17A, a member of the IL-17 family, is a major proinflammatory cytokine that has been indicated to play important roles in many immune mediated conditions, such as psoriasis and cancers (Witowski et al. (2004) Cell Mol. Life Sci. p. 567-79; Miossec and Kolls (2012) Nat. Rev. Drug Discov. p. 763-76).

In some embodiments, the IL-17 inhibitor is ixekizumab (CAS Registry Number: 1143503-69-8). Ixekizumab is also known as LY2439821. Ixekizumab is a humanized IgG4 monoclonal antibody that targets IL-17A.

Ixekizumab is described, e.g., in WO 2007/070750, U.S. Pat. Nos. 7,838,638, and 8,110,191. The heavy chain variable region of ixekizumab has the amino acid sequence of:

(SEQ ID NO: 2016)
QVQLVQSGAEVKKPGSSVKVSCKASGYSFTDYHIHWVRQAPGQGLEWMG
VINPMYGTTDYNQRFKGRVTITADESTSTAYMELSSLRSEDTAVYYCAR
YDYFTGTGVYWGQGTLVTVSS (disclosed as SEQ ID NO:
118 in WO 2007/070750).

The light chain variable region of ixekizumab has the amino acid sequence of:

(SEQ ID NO: 2017)
DIVMTQTPLSLSVTPGQPASISCRSSRSLVHSRGNTYLHWYLQKPGQSP
QLLIYKVSNRFIGVPDRFSGSGSGTDFTLKISRVEAEDVGVYYCSQSTH
LPFTFGQGTKLEIK (disclosed as SEQ ID NO: 241 in
WO 2007/070750).

In some embodiments, the IL-17 inhibitor is brodalumab (CAS Registry Number: 1174395-19-7). Brodalumab is also known as AMG 827 or AM-14. Brodalumab binds to the interleukin-17 receptor A (IL-17RA) and prevents IL-17 from activating the receptor.

Brodalumab is disclosed, e.g., in WO 2008/054603, U.S. Pat. Nos. 7,767,206, 7,786,284, 7,833,527, 7,939,070, 8,435,518, 8,545,842, 8,790,648, and 9,073,999. The heavy chain CDR1 of brodalumab has the amino acid sequence of RYGIS (SEQ ID NO: 2018) (as disclosed as SEQ ID NO: 146 in WO 2008/054603). The heavy chain CDR2 of brodalumab has the amino acid sequence of WISTYSGNTNYAQKLQG (SEQ ID NO: 2019) (as disclosed as SEQ ID NO: 147 in WO 2008/054603). The heavy chain CDR3 of brodalumab has the amino acid sequence of RQLYFDY (SEQ ID NO: 2020) (as disclosed as SEQ ID NO: 148 in WO 2008/054603). The light chain CDR1 of brodalumab has the amino acid sequence of RASQSVSSNLA (SEQ ID NO: 2021) (as disclosed as SEQ ID NO: 224 in WO 2008/054603). The heavy chain CDR2 of brodalumab has the amino acid sequence of DASTRAT (SEQ ID NO: 2022) (as disclosed as SEQ ID NO: 225 in WO 2008/054603). The heavy chain CDR3 of brodalumab has the amino acid sequence of QQYDNWPLT (SEQ ID NO: 2023) (as disclosed as SEQ ID NO: 226 in WO 2008/054603).

### IL-1β Inhibitor

In some embodiments, the BCMA CAR-expressing cell therapy is administered in combination with an interleukine-1 beta (IL-113) inhibitor.

The Interleukin-1 (IL-1) family of cytokines is a group of secreted pleotropic cytokines with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). The IL-1 family comprises, inter alia, IL-1 beta (IL-1β), and IL-1alpha (IL-1a). IL-1β is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1β, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1β results in a decrease in tumor burden and metastasis (Voronov et al. (2003) Proc. Natl. Acad. Sci. U.S.A. p. 2645-50).

In some embodiments, the IL-1β inhibitor is chosen from Anakinra or Rilonacept.

In some embodiments, the IL-1β inhibitor is Anakinra (Amgen), also known as Kineret. Anakinra is an IL-1Ra antagonist that competes with IL-1β for binding to the cell surface receptor.

In some embodiments, the IL-1β inhibitor is Rilonacept (Regeneron), also known as Arcalyst. Rilonacept is a fusion protein consisting of the ligand-binding domains of the extracellular portions of the human interleukin-1 receptor component (IL-1R1) and IL-1 receptor accessory protein (IL-1RAcP) linked to the fragment-crystallizable portion (Fc region) of human IgG1. Rilonacept is an IL-1β inhibitor which, e.g., binds and neturalizes IL-1.

### CD32B Inhibitor

In some embodiments, the BCMA CAR-expressing cell therapy is administered in combination with CD32B inhibitor.

In some embodiments, the CD32B inhibitor is an anti-CD32B antibody molecule. Exemplary anti-CD32B antibody molecules are disclosed in U.S. Pat. Nos. 8,187,593, 8,778,339, 8,802,089, US20060073142, US20170198040, US20130251706, and WO2009083009, herein incorporated by reference in their entirety. In some embodiments, the anti-CD32B antibody molecule is an antibody molecule disclosed in US20170198040.

### Chemotherapeutic Agents

In some embodiments, the BCMA CAR-expressing cell therapy is administered in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4.9}]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine- (SEQ ID NO: 2035), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

### Additional Agents for Combination

TABLE 27
Selected therapeutic agents that can be administered in combination with the BCMA CAR-
expressing cell therapy, e.g., as a single agent or in combination with other agents described herein.
Each publication listed in this Table is herein incorporated by reference in its entirety, including all
structural formulae therein.

| | Generic | | Patents/Patent |
|---|---|---|---|
| Compound | Name | | Application |
| Designation | Tradename | Compound Structure | Publications |
| A1 | Sotrastaurin | 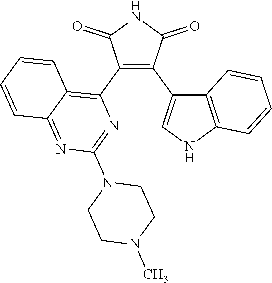 [SMILES: CN1CCN(c2nc(C3=C(c4c[nH]c5ccccc45)C(=O)NC3=O)c3ccccc3n2)CC1] | EP 1682103 US 2007/142401 WO 2005/039549 |
| A2 | Nilotinib HCl monohydrate TASIGNA ® | 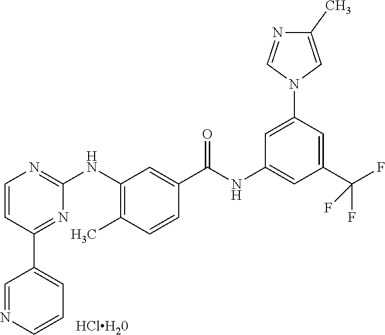 | WO 2004/005281 U.S. Pat. No. 7,169,791 |
| A7 | | 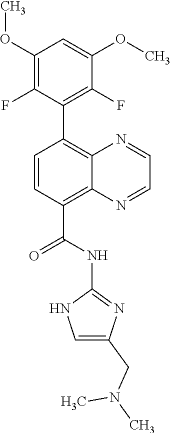 [SMILES: COc1cc(OC)c(F)c(-c2ccc(C(=O)Nc3nc(CN(C)C)c[nH]3)c3nccnc23)c1F] | WO 2009/141386 US 2010/0105667 |
| A8 | | 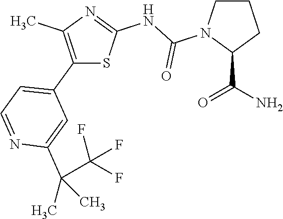 [SMILES: Cc1nc(NC(=O)N2CCC[C@H]2C(N)=O)sc1-c1ccnc(C(C)(C)C(F)(F)F)c1] | WO 2010/029082 |
| A10 | | 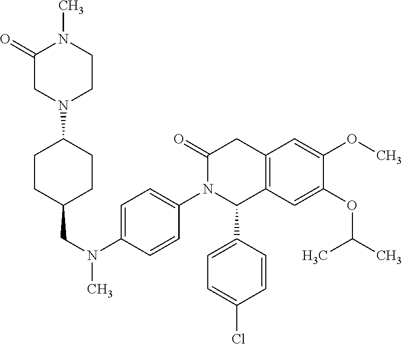 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2] | WO 2011/076786 |
| A11 | Deferasirox EXJADE ® | 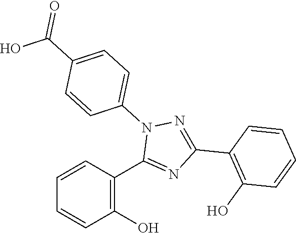 [SMILES: O=C(O)c1ccc(-n2nc(-c3ccccc3O)nc2-c2ccccc2O)cc1] | WO 1997/049395 |
| A12 | Letrozole FEMARA ® | 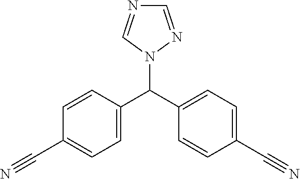 [SMILES: N#Cc1ccc(C(c2ccc(C#N)cc2)n2cncn2)cc1] | U.S. Pat. No. 4,978,672 |
| A13 | | 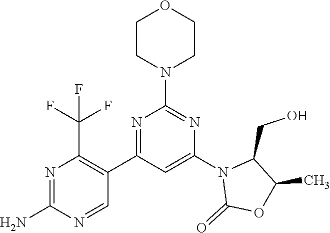 [SMILES: C[C@H]1OC(=O)N(c2cc(-c3cnc(N)nc3C(F)(F)F)nc(N3CCOCC3)n2)[C@H]1CO] | WO 2013/124826 US 2013/0225574 |
| A14 | | 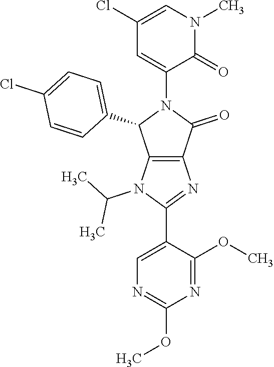 [SMILES: COc1ncc(-c2nc3c(n2C(C)C)[C@H](c2ccc(Cl)cc2)N(c2cc(Cl)cn(C)c2=O)C3=O)c(OC)n1] | WO 2013/111105 |
| A15 | | 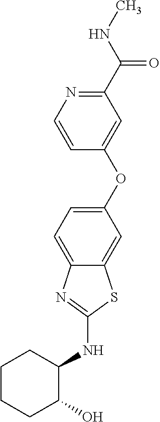 [SMILES: CNC(=O)c1cc(Oc2ccc3nc(N[C@@H]4CCCC[C@H]4O)sc3c2)ccn1] | WO 2007/121484 |
| A16 | Imatinib mesylate GLEEVEC ® | 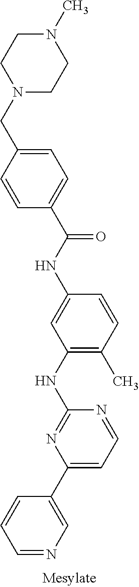 [SMILES: Cc1ccc(NC(=O)c2ccc(CN3CCN(C)CC3)cc2)cc1Nc1nccc(-c2cccnc2)n1] | WO 1999/003854 |
| A17 | | 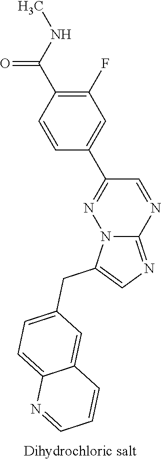 [SMILES: CNC(=O)c1ccc(-c2cnc3ncc(Cc4ccc5ncccc5c4)n3n2)cc1F] | EP 2099447 U.S. Pat. No. 7,767,675 U.S. Pat. No. 8,420,645 |
| A18 | Ruxolitinib Phosphate JAKAFI ® | 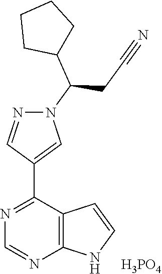 | WO 2007/070514 EP 2474545 U.S. Pat. No. 7,598,257 WO 2014/018632 |
| A19 | Panobinostat | 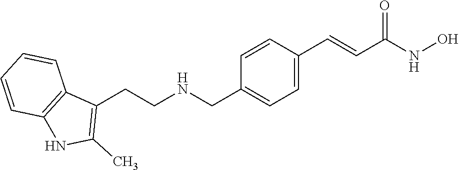 [SMILES: Cc1[nH]c2ccccc2c1CCNCc1ccc(/C=C/C(=O)NO)cc1] | WO 2014/072493 WO 2002/022577 EP 1870399 |
| A20 | Osilodrostat | 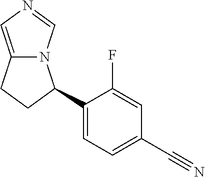 [SMILES: N#Cc1ccc([C@H]2CCc3cncn32)c(F)c1] | WO 2007/024945 |
| A21 | | 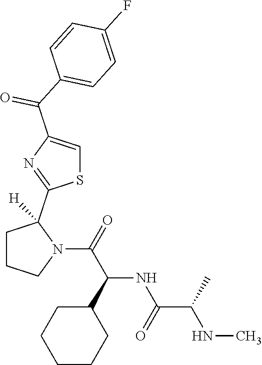 [SMILES: CN[C@@H](C)C(=O)N[C@H](C(=O)N1CCC[C@H]1c1nc(C(=O)c2ccc(F)cc2)cs1)C1CCCCC1] | WO 2008/016893 EP 2051990 U.S. Pat. No. 8,546,336 |
| A23 | ceritinib ZYKADIA ™ | 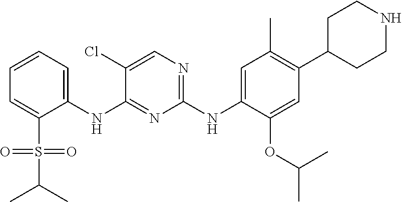 [SMILES: Cc1cc(Nc2ncc(Cl)c(Nc3ccccc3S(=O)(=O)C(C)C)n2)c(OC(C)C)cc1C1CCNCC1] | WO 2008/073687 U.S. Pat. No. 8,039,479 |
| A24 | | 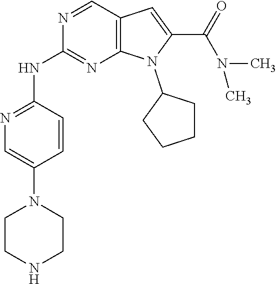 [SMILES: CN(C)C(=O)c1cc2cnc(Nc3ccc(N4CCNCC4)cn3)nc2n1C1CCCC1] | U.S. Pat. No. 8,415,355 U.S. Pat. No. 8,685,980 |
| A25 | | 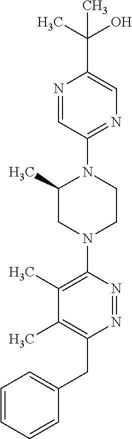 [SMILES: Cc1c(Cc2ccccc2)nnc(N2CCN(c3cnc(C(C)(C)O)cn3)[C@H](C)C2)c1C] | WO 2010/007120 |
| A26 | | Human monoclonal antibody to PRLR | U.S. Pat. No. 7,867,493 |
| A27 | | 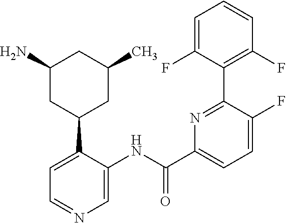 [SMILES: C[C@@H]1C[C@H](N)C[C@H](c2ccncc2NC(=O)c2ccc(F)c(-c3c(F)cccc3F)n2)C1] | WO 2010/026124 EP 2344474 US 2010/0056576 WO2008/106692 |
| A28 | | 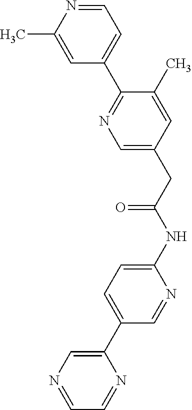 [SMILES: Cc1cc(-c2ncc(CC(=O)Nc3ccc(-c4cnccn4)cn3)cc2C)ccn1] | WO 2010/101849 |
| A30 | | 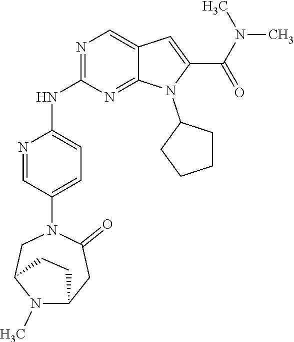 [SMILES: CN(C)C(=O)c1cc2cnc(Nc3ccc(N4C[C@H]5CC[C@@H](CC4=O)N5C)cn3)nc2n1C1CCCC1] | WO 2011/101409 |
| A31 | | Human monoclonal antibody to HER3 | WO 2012/022814 |
| | | | EP 2606070 |
| | | | U.S. Pat. No. 8,735,551 |
| A32 | | Antibody Drug Conjugate (ADC) | WO 2014/160160 |
| | | | Ab: 12425 (see |
| | | | Table 1, paragraph |
| | | | [00191]) |
| | | | Linker: SMCC (see |
| | | | paragraph [00117] |
| | | | Payload: DM1 (see |
| | | | paragraph [00111] |
| | | | See also Claim 29 |
| A33 | | Monoclonal antibody or Fab to M-CSF | WO 2004/045532 |
| A35 | Midostaurin | 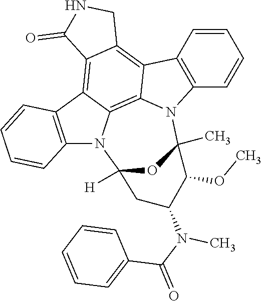 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4] | WO 2003/037347 EP 1441737 US 2012/252785 |
| A36 | Everolimus AFINITOR ® | 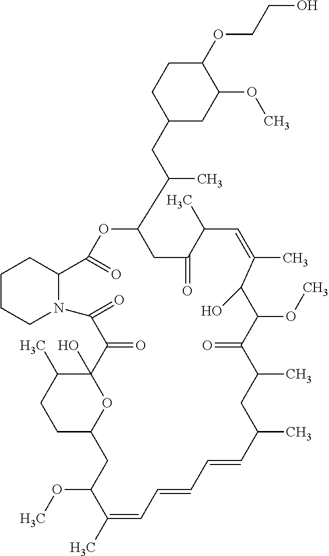 [SMILES: COC1CC2CCC(C)C(O)(O2)C(=O)C(=O)N2CCCCC2C(=O)OC(C(C)CC2CCC(OCCO)C(OC)C2)CC(=O)C(C)/C=C(/C)C(O)C(OC)C(=O)C(C)CC(C)/C=C/C=C/C=C\1C] | WO 2014/085318 |
| A37 | | 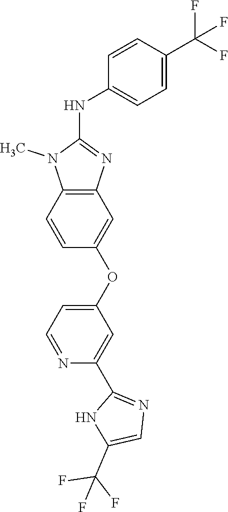 [SMILES: Cn1c(Nc2ccc(C(F)(F)F)cc2)nc2cc(Oc3ccnc(-c4ncc(C(F)(F)F)[nH]4)c3)ccc21] | WO 2007/030377 U.S. Pat. No. 7,482,367 |
| A38 | Pasireotide diaspartate SIGNIFOR ® | 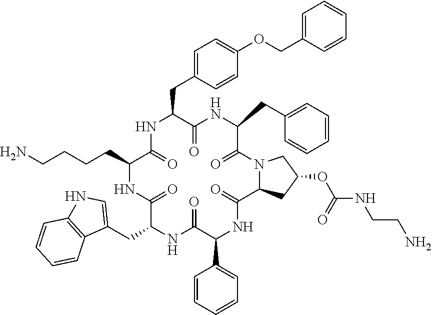 [SMILES: NCCCC[C@@H]1NC(=O)[C@@H](Cc2c[nH]c3ccccc23)NC(=O)[C@H](c2ccccc2)NC(=O)[C@@H]2C[C@@H](OC(=O)NCCN)CN2C(=O)[C@H](Cc2ccccc2)NC(=O)[C@H](Cc2ccc(OCc3ccccc3)cc2)NC1=O] | WO 2002/010192 U.S. Pat. No. 7,473,761 |
| A40 | | 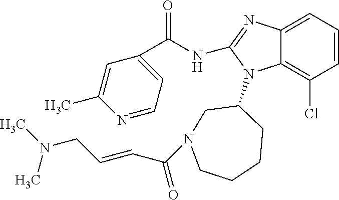 [SMILES: Cc1cc(C(=O)Nc2nc3cccc(Cl)c3n2[C@@H]2CCCCN(C(=O)/C=C/CN(C)C)C2)ccn1] | WO 2013/184757 |
| A41 | | 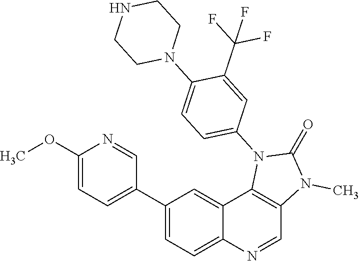 | WO 2006/122806 |
| A42 | | 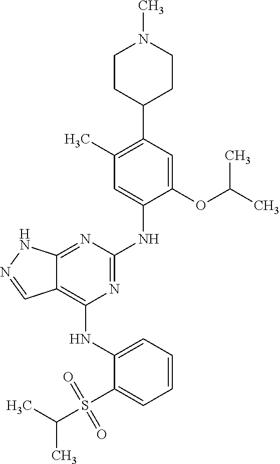 [SMILES: Cc1cc(Nc2nc(Nc3ccccc3S(=O)(=O)C(C)C)c3cn[nH]c3n2)c(OC(C)C)cc1C1CCN(C)CC1] | WO 2008/073687 U.S. Pat. No. 8,372,858 |
| A43 | | 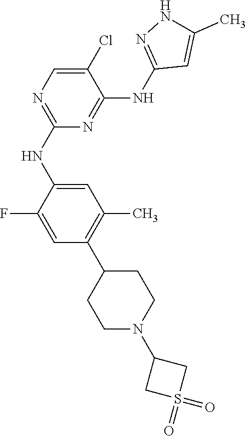 [SMILES: Cc1cc(Nc2nc(Nc3cc(C)c(C4CCN(C5CS(=O)(=O)C5)CC4)cc3F)ncc2Cl)n[nH]1] | WO 2010/002655 U.S. Pat. No. 8,519,129 |
| A44 | | 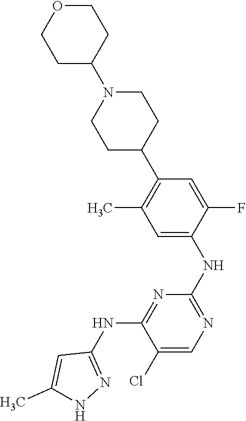 [SMILES: Cc1cc(Nc2nc(Nc3cc(C)c(C4CCN(C5CCOCC5)CC4)cc3F)ncc2Cl)n[nH]1] | WO 2010/002655 U.S. Pat. No. 8,519,129 |
| A45 | | 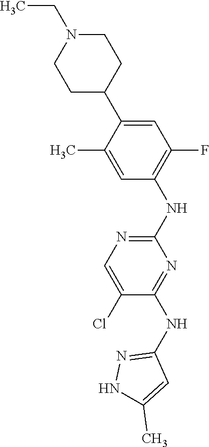 [SMILES: CCN1CCC(c2cc(F)c(Nc3ncc(Cl)c(Nc4cc(C)[nH]n4)n3)cc2C)CC1] | WO 2010/002655 |
| A46 | Valspodar AMDRAY ™ | 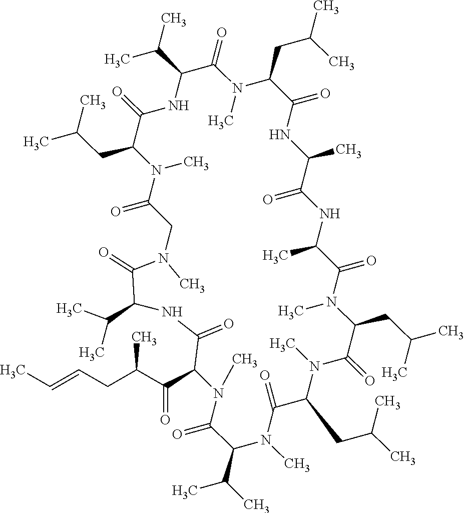 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] | EP 296122 |
| A47 | Vatalanib succinate | 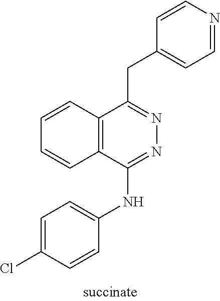 [SMILES: Clc1ccc(Nc2nnc(Cc3ccncc3)c3ccccc23)cc1] | WO 98/35958 |
| A48 | | IDH inhibitor | WO2014/141104 |
| A49 | | 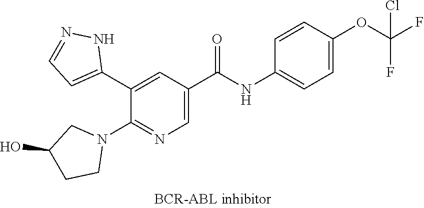 [SMILES: O=C(Nc1ccc(OC(F)(F)Cl)cc1)c1cnc(N2CC[C@@H](O)C2)c(-c2ccn[nH]2)c1] | WO2013/171639 WO2013/171640 WO2013/171641 WO2013/171642 |
| A50 | | cRAF inhibitor | WO2014/151616 |
| A51 | | ERK1/2 ATP competitive inhibitor | WO2015/066188 |

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic.

Examples of suitable biopolymers include, but are not limited to, agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, CAR-expressing cells described herein are seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of a CAR-expressing cell, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97-101; and WO2014/110591.

### Pharmaceutical Compositions and Treatments

Pharmaceutical compositions of the present invention may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988)..

In certain aspects, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present invention are administered to a patient by intradermal or subcutaneous injection. In one aspect, the CAR-expressing cell (e.g., T cell or NK cell) compositions of the present invention are administered by i.v. injection. The compositions of CAR-expressing cells (e.g., T cells or NK cells) may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., immune effector cells (e.g., T cells or NK cells). These immune effector cell (e.g., T cell or NK cell) isolates may be expanded by methods known in the art and treated such that one or more CAR constructs of the invention may be introduced, thereby creating a CAR-expressing cell (e.g., CAR T cell or CAR-expressing NK cell)of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cells (e.g., CAR T cells or NK cells) of the present invention. In an additional aspect, expanded cells are administered before or following surgery.

In embodiments, lymphodepletion is performed on a subject, e.g., prior to administering one or more cells that express a CAR described herein, e.g., a BCMA-binding CAR described herein. In embodiments, the lymphodepletion comprises administering one or more of melphalan, cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the CAR is introduced into immune effector cells (e.g., T cells or NK cells), e.g., using in vitro transcription, and the subject (e.g., human) receives an initial administration of CAR immune effector cells (e.g., T cells or NK cells)of the invention, and one or more subsequent administrations of the CAR immune effector cells (e.g., T cells or NK cells) of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR immune effector cells (e.g., T cells or NK cells) of the invention are administered to the subject (e.g., human) per week, e.g., 2, 3, or 4 administrations of the CAR immune effector cells (e.g., T cells or NK cells) of the invention are administered per week. In one embodiment, the subject (e.g., human subject) receives more than one administration of the CAR immune effector cells (e.g., T cells or NK cells) per week (e.g., 2, 3 or 4 administrations per week) (also referred to herein as a cycle), followed by a week of no CAR immune effector cells (e.g., T cells or NK cells) administrations, and then one or more additional administration of the CAR immune effector cells (e.g., T cells or NK cells) (e.g., more than one administration of the CAR immune effector cells (e.g., T cells or NK cells) per week) is administered to the subject. In another embodiment, the subject (e.g., human subject) receives more than one cycle of CAR immune effector cells (e.g., T cells or NK cells), and the time between each cycle is less than 10, 9, 8, 7, 6, 5, 4, or 3 days. In one embodiment, the CAR immune effector cells (e.g., T cells or NK cells) are administered every other day for 3 administrations per week. In one embodiment, the CAR immune effector cells (e.g., T cells or NK cells) of the invention are administered for at least two, three, four, five, six, seven, eight or more weeks.

In one aspect, BCMA CAR-expressing cells (e.g., BCMA CARTs or BCMA CAR-expressing NK cells) are generated using lentiviral viral vectors, such as lentivirus. CAR-expressing cells (e.g., CARTs or CAR-expressing NK cells) generated that way will have stable CAR expression.

In one aspect, CAR-expressing cells, e.g., CARTs, are generated using a viral vector such as a gammaretroviral vector, e.g., a gammaretroviral vector described herein. CARTs generated using these vectors can have stable CAR expression.

In one aspect, CAR-expressing cells (e.g., CARTs or CAR-expressing NK cells) transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the cell, e.g., T cell or NK cell, by electroporation.

A potential issue that can arise in patients being treated using transiently expressing CAR-expressing cells (e.g., CARTs or CAR-expressing NK cells) (particularly with murine scFv bearing CAR-expressing cells (e.g., CARTs or CAR-expressing NK cells)) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CAR-expressing cell (e.g., CART or CAR-expressing NK cell) infusion breaks should not last more than ten to fourteen days.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples specifically point out various aspects of the present invention, and are not to be construed as limiting in any way the remainder of the disclosure.

### Example 1: BCMA-CART in Multiple Myeloma

### CART-BCMA (MCM998) Demonstrates Potent Anti-Tumor Activity In Vivo

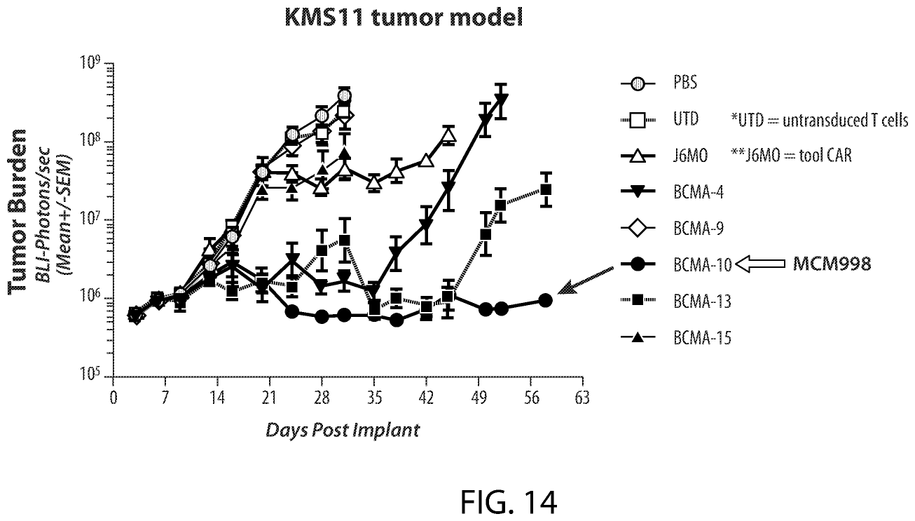

The level of tumor burden in a KMS11 tumor model following infusion of PBS, untransduced T cells (“UTD”), or T cells transduced with either a tool CAR (“J6MO”), BCMA-4, BCMA-9, BCMA-10 (“MCM998”), BCMA-13, or BCMA-15 was evaluated. BCMA-10 demonstrated the most potent anti-tumor activity (FIG. 14).

### CART-BCMA in Multiple Myeloma Clinical Trial (NCT Number: NCT02546167; UPCC 14415)

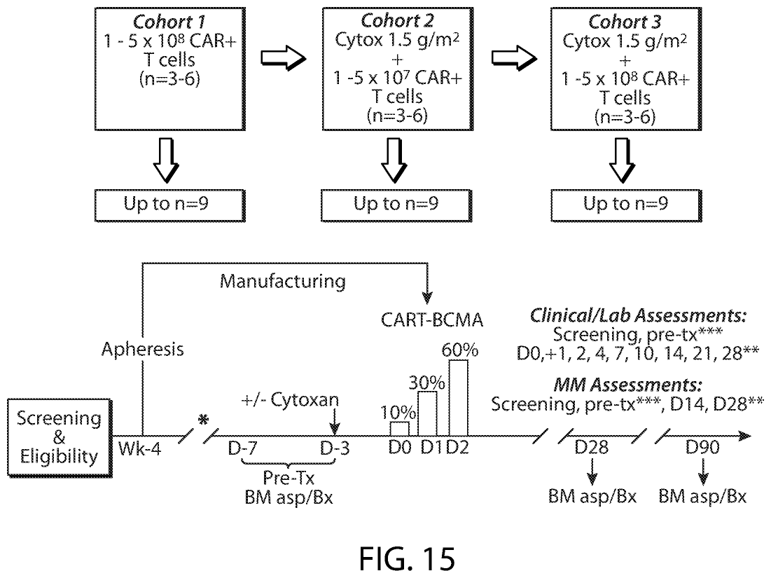

An open-label, single-center, pilot study to assess the safety and feasibility of infusion of autologous T cells expressing BCMA-specific chimeric antigen receptors with tandem 4-1BB and CD3zeta signaling domains (referred to herein as “CART-BCMA”) in adult patients with multiple myeloma (MM) was designed (FIG. 15).

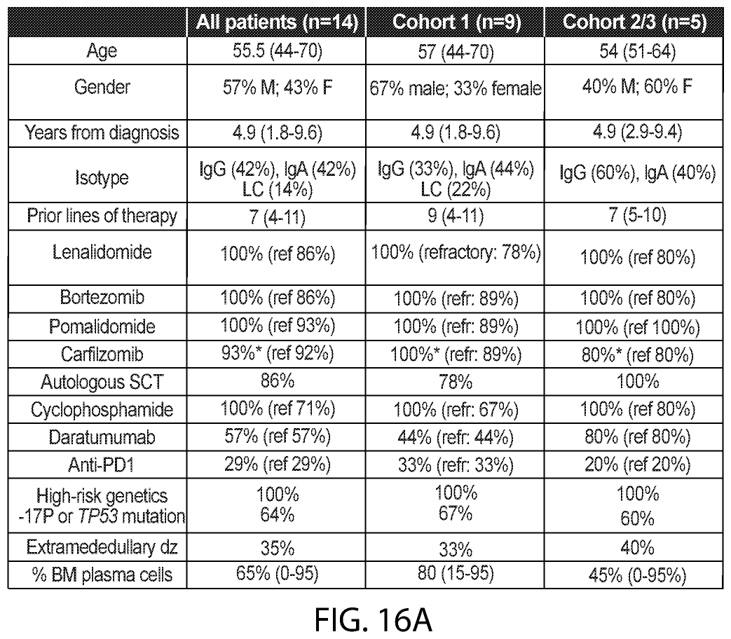

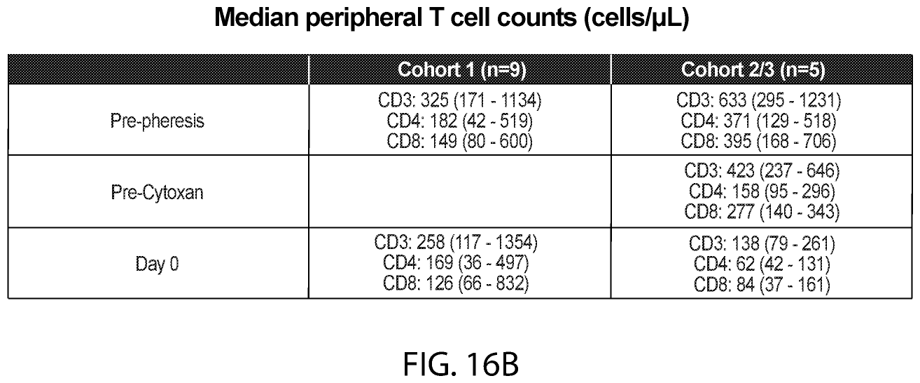

Patients were divided into three groups (FIG. 15). Cohort 1 patients received 1-5×10⁸ CART-BCMA cells given as a split dose infusion over 3 days. Cohort 2 patients received a cyclophosphamide infusion prior to administration of 1-5×10⁷CART-BCMA cells given as a split dose infusion over 3 days. Cohort 3 patients received a cyclophosphamide infusion prior to administration of 1-5×10⁸ CART-BCMA cells given as a split dose infusion over 3 days. FIG. 16A provides patient disease characteristics. FIG. 16B provides information on presence of baseline lymphopenia due to disease and prior therapies.

### CART-BCMA (MCM998) Manufacturing and Dosing

All the CART-BCMA products were successfully manufactured with minimum target threshold dose. Manufactured products had a median transduction efficiency of 22.5% (9.6-33.3%); a median fold expansion of 20.7 (7.9-60.4); median population doublings of 4.4 (2.98-5.92); median pheresis CD4/CD8 ratio of 1.03 (0.61-3.2); and median product CD4/CD8 ratio of 1.72 (0.84-3.9). Thirteen of the fourteen patients achieved max target dose of 5×10⁸ or 5×10⁷. Patient 2 of Cohort 1 received 1.9×10⁸ cells, including 69% T cells in the product. Twelve of the fourteen patients received 100% of the planned dose. Patient 1 and Patient 3 of Cohort 1 only received the first two infusions (40%) due to fevers on Day 2. These data demonstrate that CART-BCMA manufacturing is feasible in heavily pre-treated multiple myeloma patients.

### Clinical Outcomes

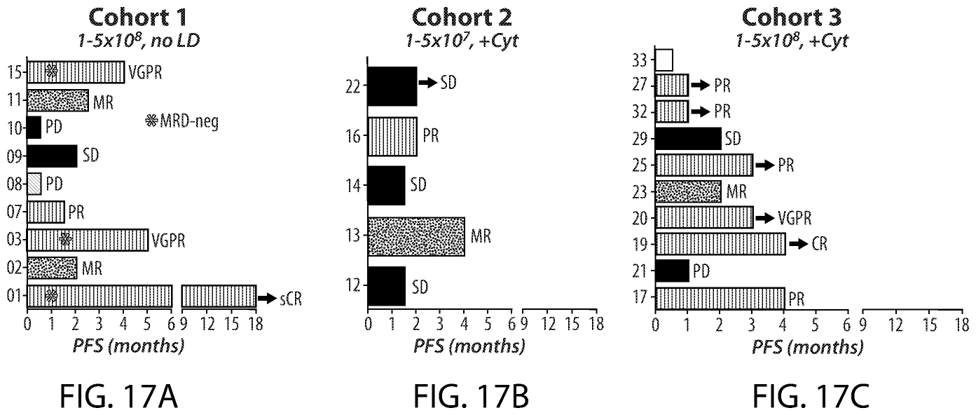

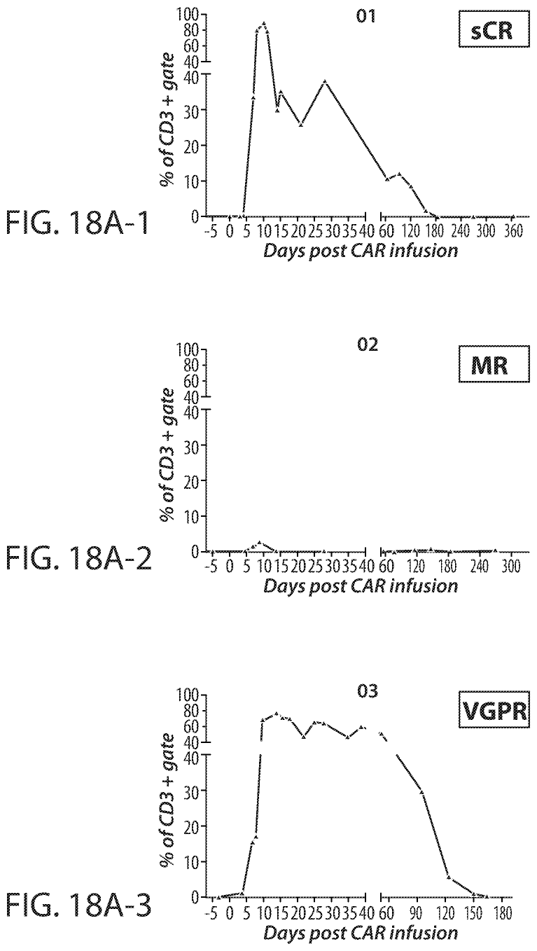

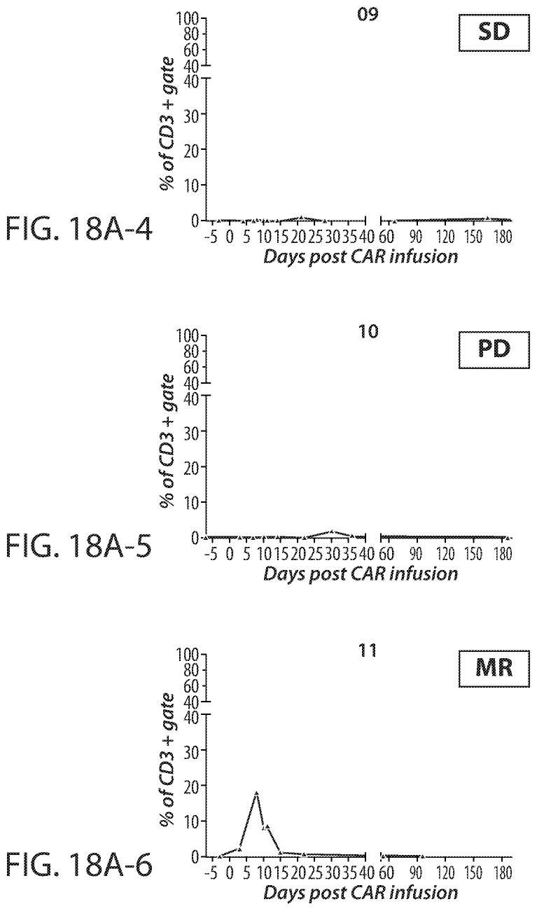

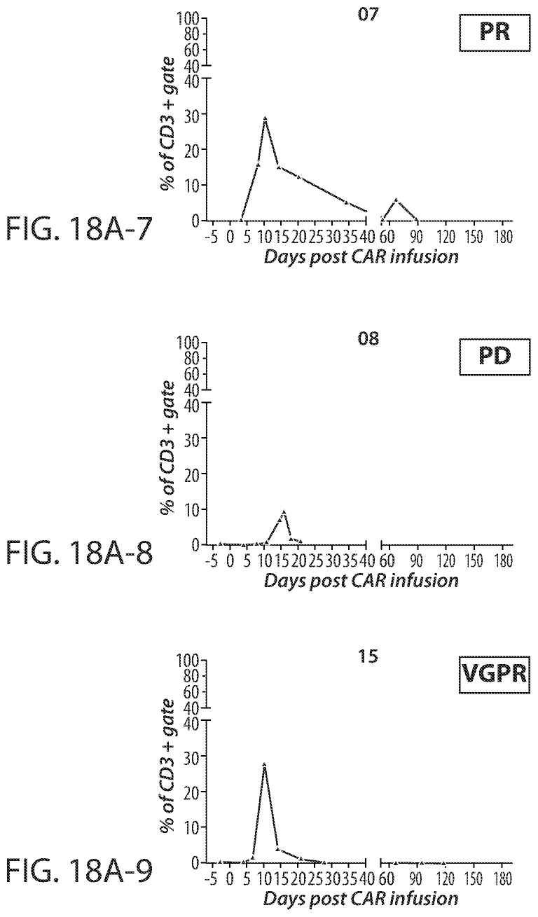

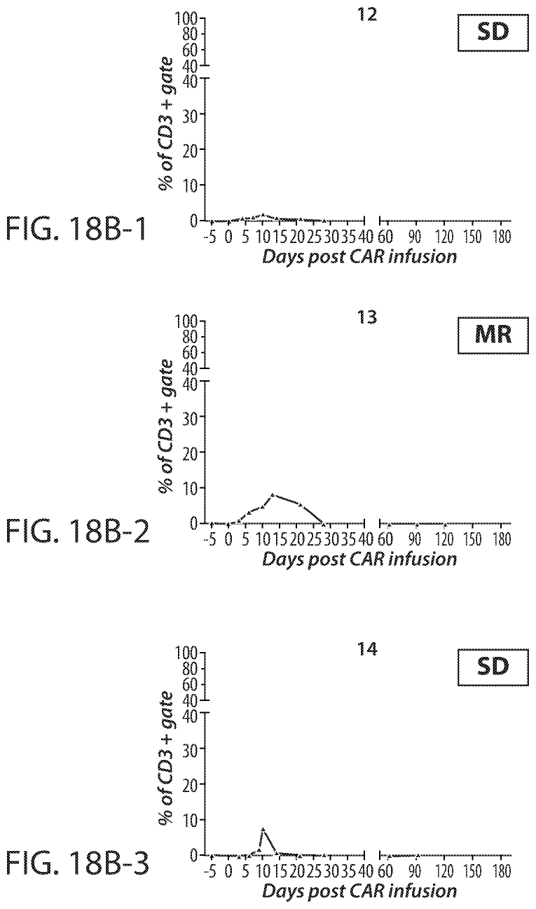

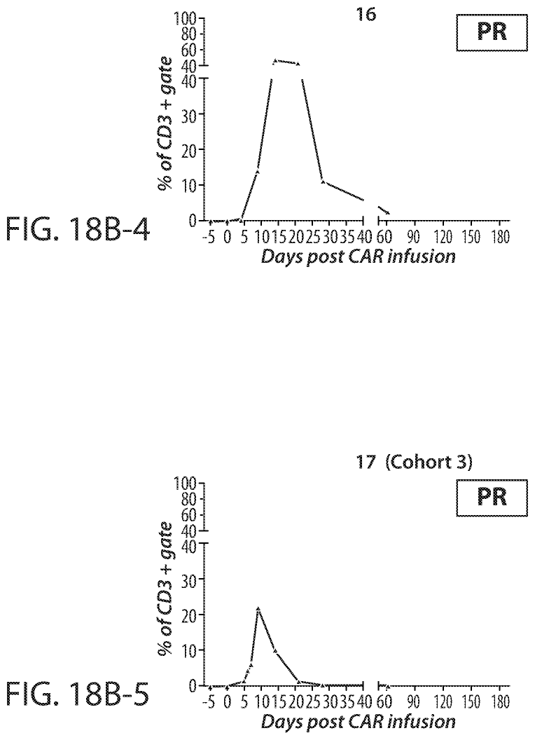

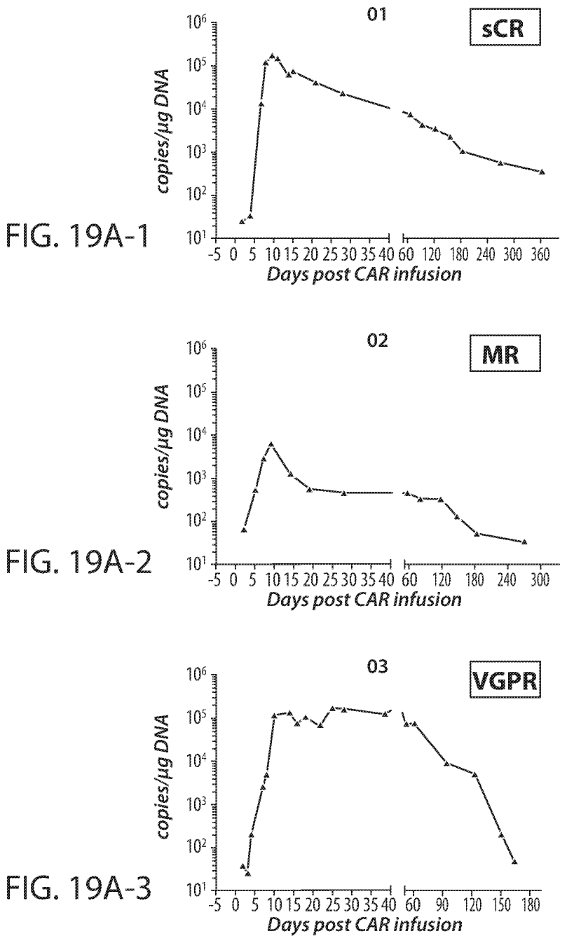

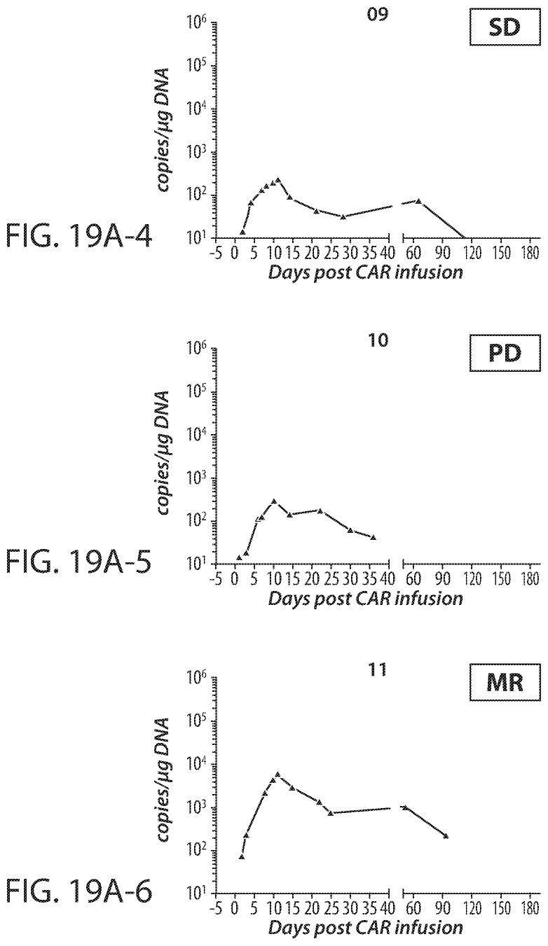

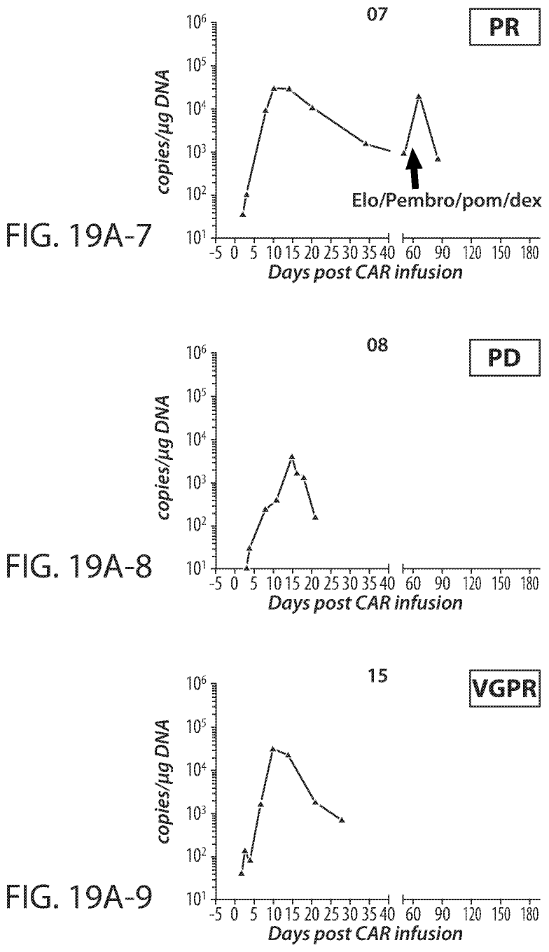

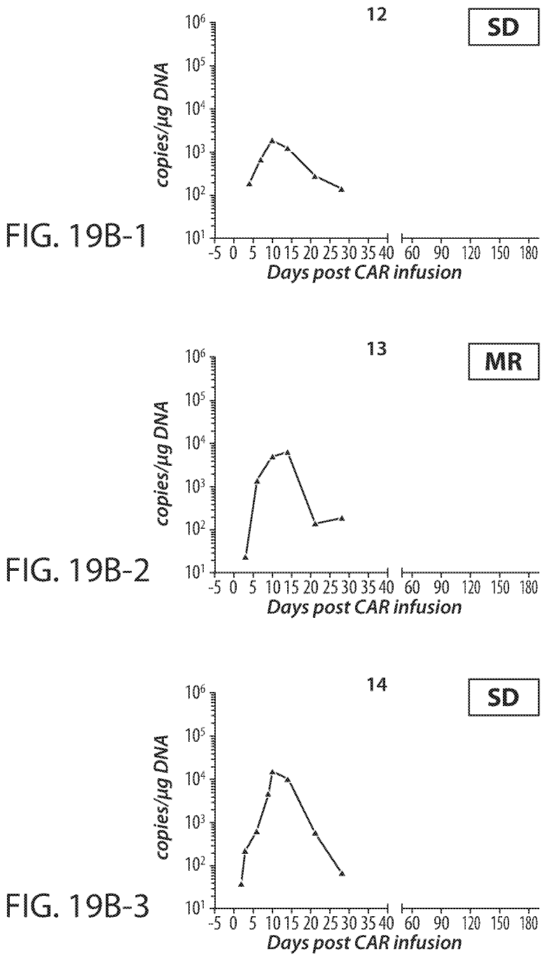

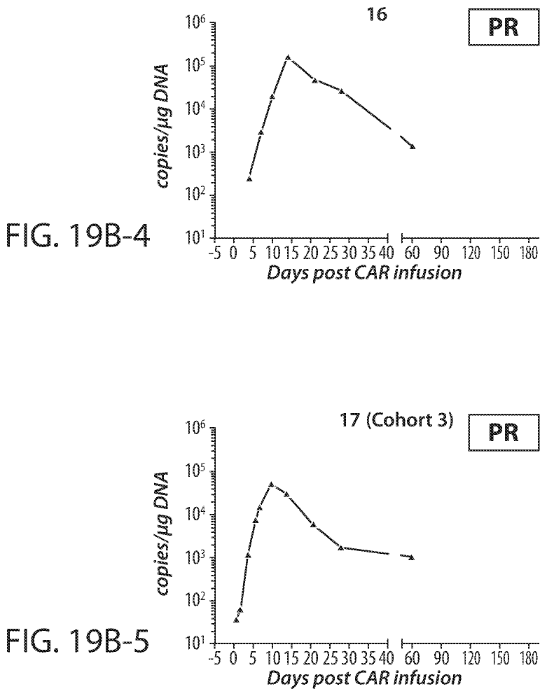

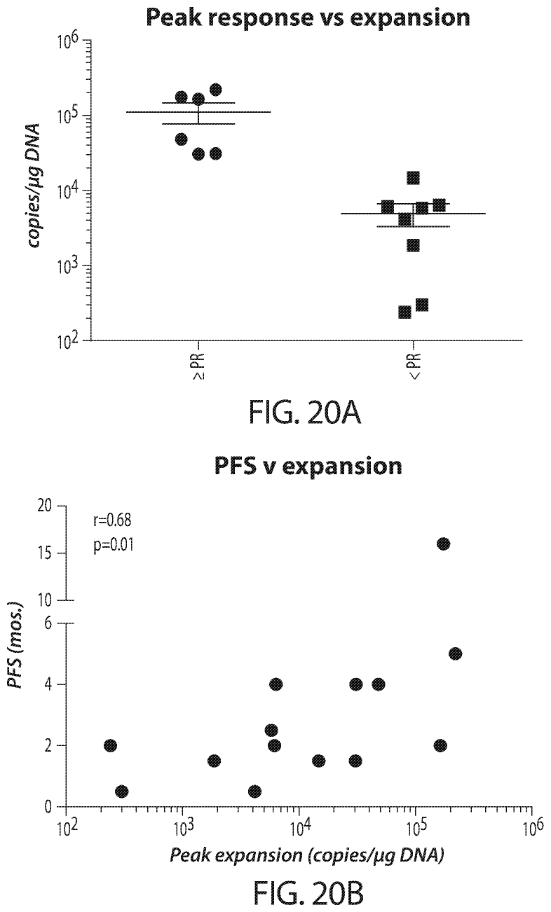

Clinical activity is shown in FIGS. 17A, 17B, and 17C for Cohorts 1, 2, and 3, respectively. The expansion of CART-BCMA was evaluated by flow cytometry (FIGS. 18A and 18B) and by PCR (FIGS. 19A and 19B). In vivo expansion of CART-BCMA may correlate with and predict response to therapy (FIGS. 20A and 20B). No correlation was observed between BCMA surface expression on multiple myeloma cells as determined by flow cytometry and clinical outcomes (data not show).

### Example 2: Correlative Analysis of Clinical Trial Data

To identify biomarkers predictive of patient response to CART-BCMA treatment, the parameters listed in Table 28 were analyzed.

TABLE 28
Biomarker parameters

| Parameter | Sample type | Technology |
|---|---|---|
| Release profile: | Product | Several: Expansion, T cell |
| QC and | | count, CD4/CD8, CAR+, IFN-γ, |
| informational | | cytotox |
| CART PK | Bone marrow | Flow, qPCR |
| | (BM), blood | |
| Serum cytokines | Blood | MSD/Luminex |
| Immunophenotype | Apheresis and | Flow |
| | Product | |
| Gene signature | Apheresis and | RNAseq |
| analysis | Product | |
| TCR diversity | Apheresis and | Deep sequencing |
| | Product | |
| Serum proteins | Serum: pre and | Somascan |
| | post infusion | |
| Target expression | Bone marrow | ELISA: BCMA levels, soluble |
| (surface and | (BM), blood | ligands |
| soluble) | | |
| (CAR) T-cell tumor | Bone marrow | ISH/IHC |
| infiltration/ | (BM) | |
| suppression | | |

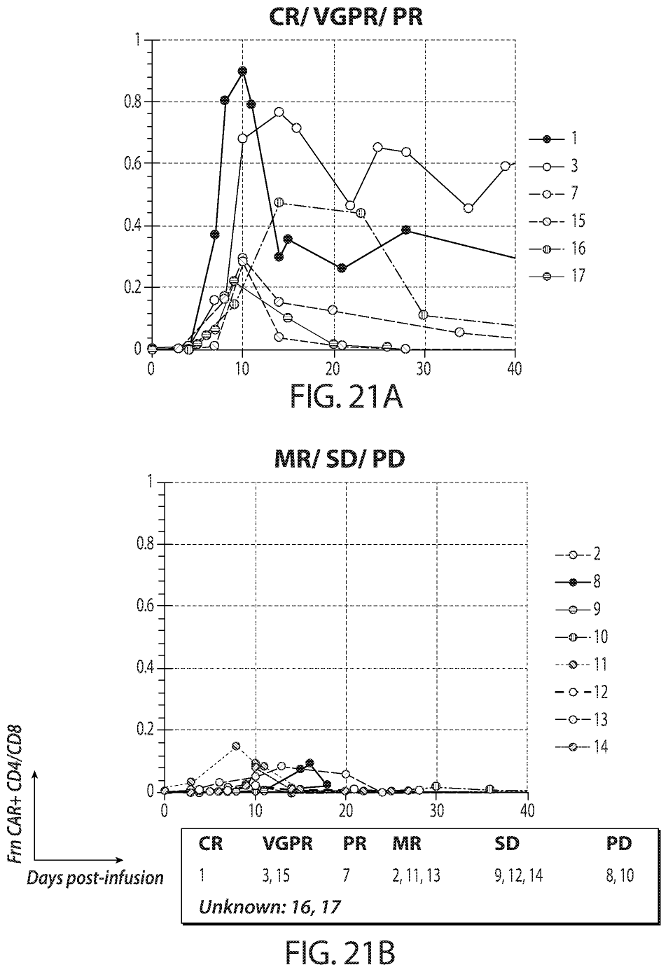

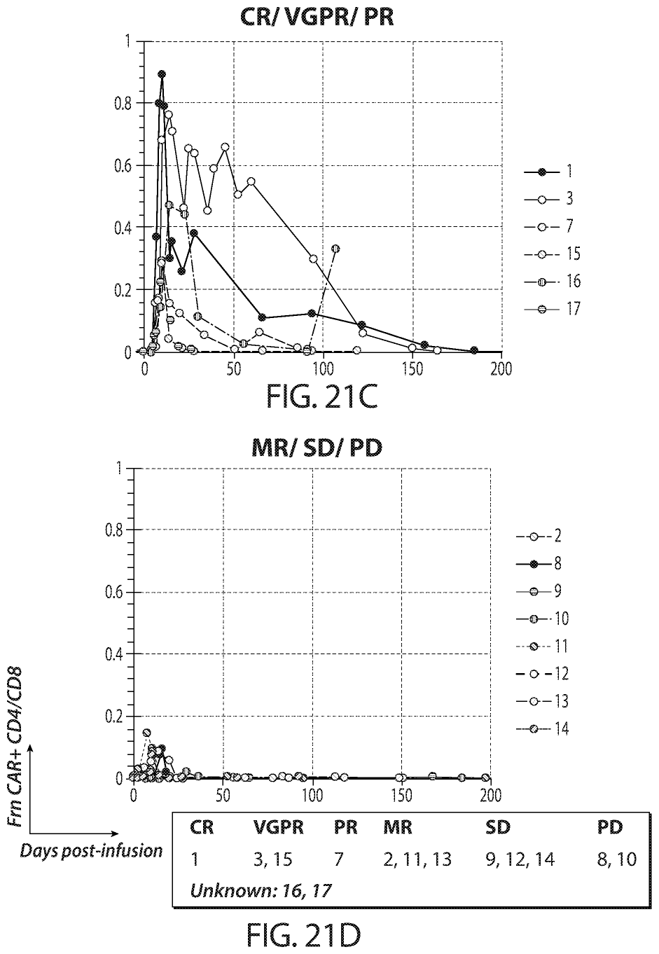

The fraction of CAR+ CD4/CD8 cells in patient samples was acquired at various time-points post-CART-BCMA infusion for Responders (patient having complete response (CR), very good partial response (VGPR), or partial response (PR)) and Non-Responders (patients having minor response (MR), stable disease (SD), or progressive disease (PD)) (FIGS. 21A, 21B, 21C, and 21D). These data demonstrate that Responders had greater persistence of CAR+ CD4/CD8 cell populations over time, as compared to Non-responders.

### Evaluation of Cytokine Levels

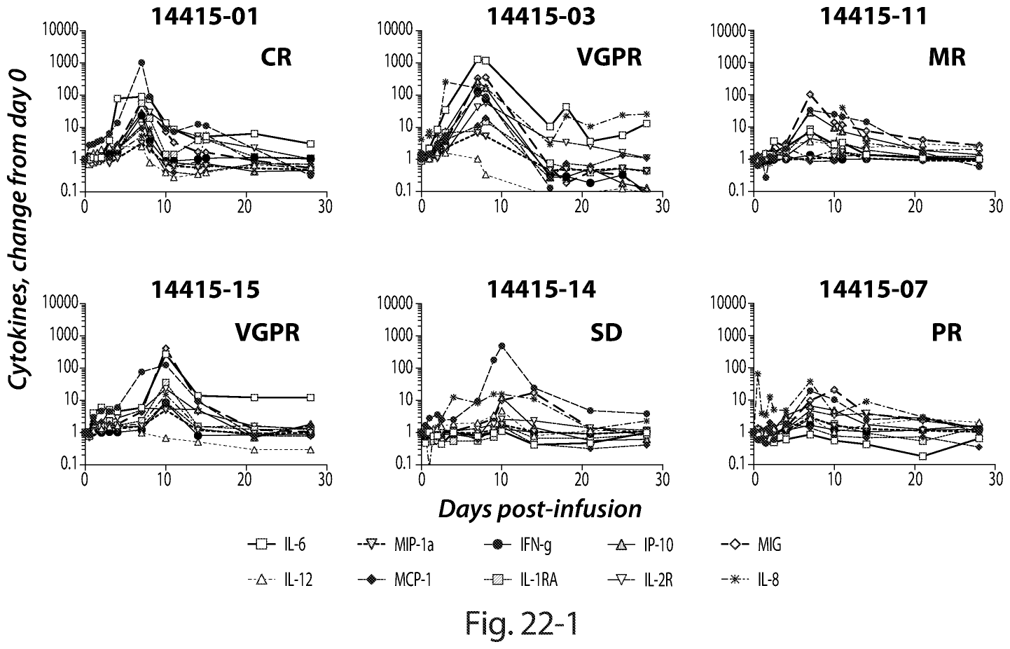

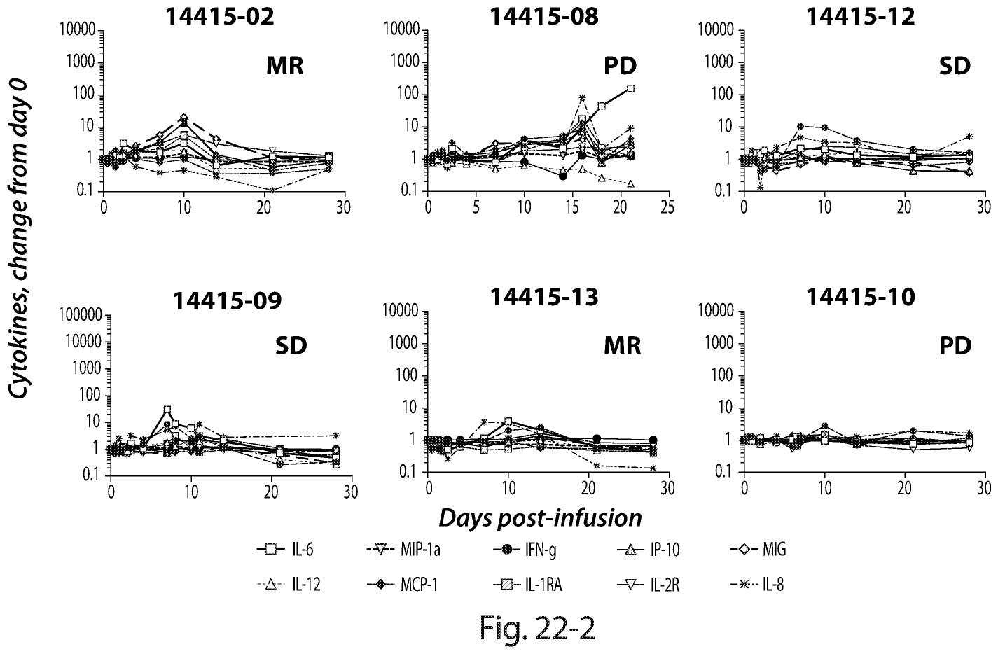

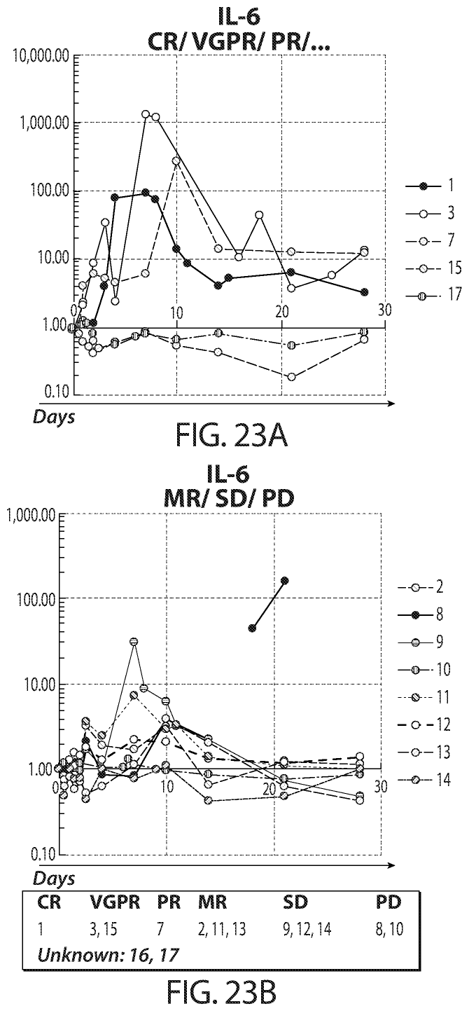

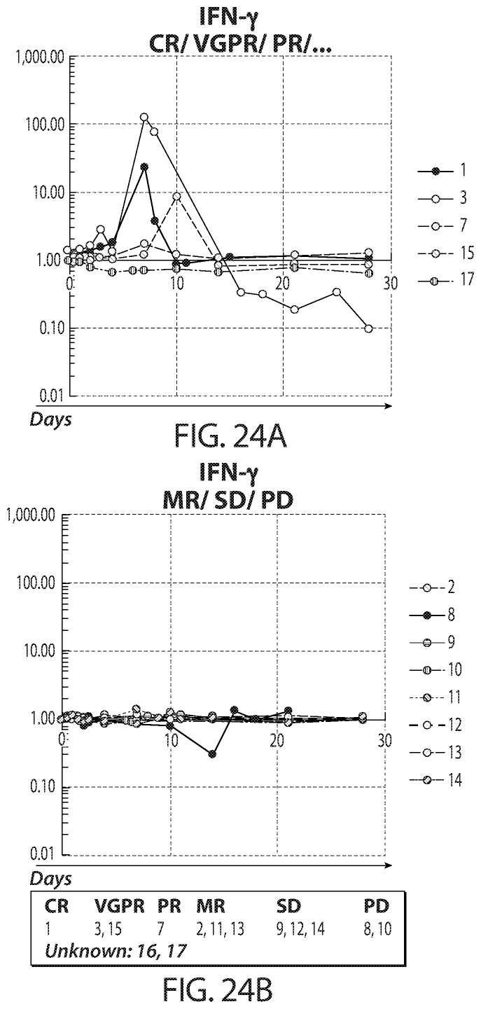

The change in level of cytokine expression at various time points post infusion of CART-BCMA was evaluated (FIG. 22). These data demonstrate that the greatest changes from baseline (Day 0) occurred in the level of IL-6 (FIGS. 23A and 23B), IL-10, monokine induced by gamma interferon (MIG), and IFN-γ (FIGS. 24A and 24B). Importantly, IFN-γ may differentiate Responders to CART-BCMA treatment from Non-Responders.

### Evaluation of BCMA Levels in Serum

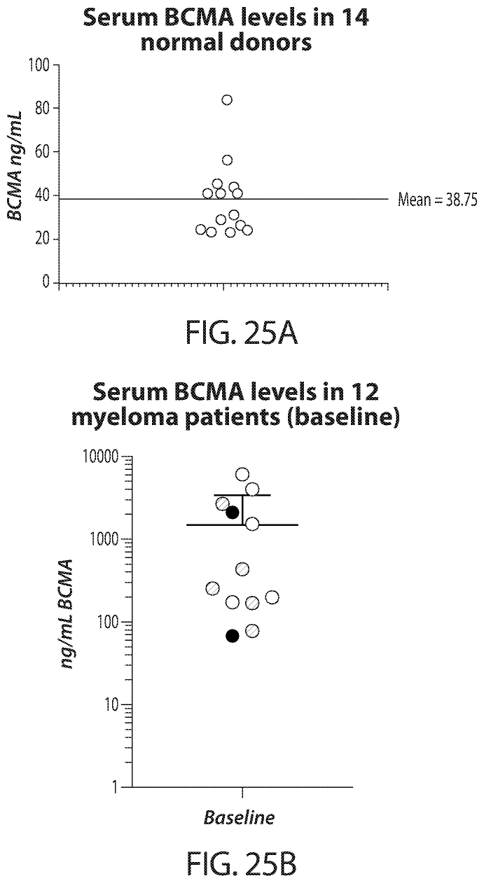

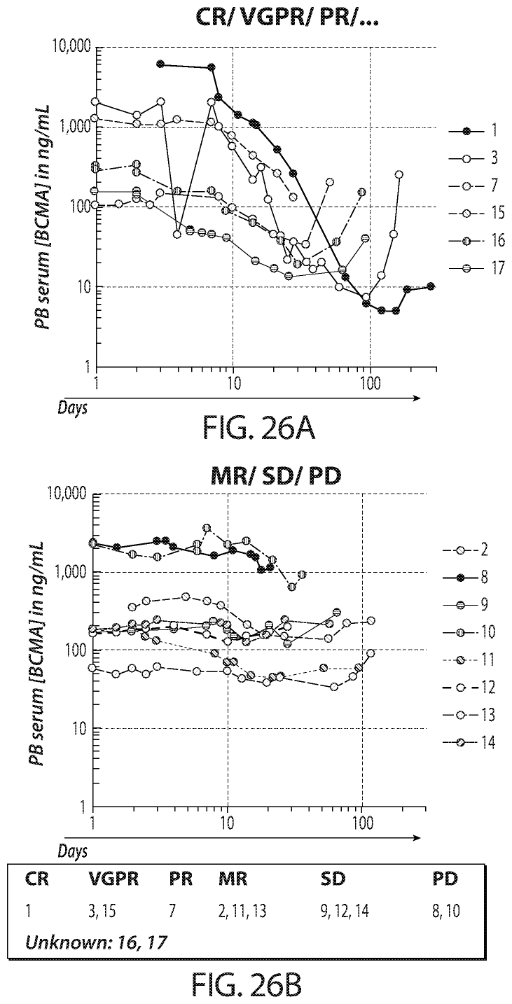

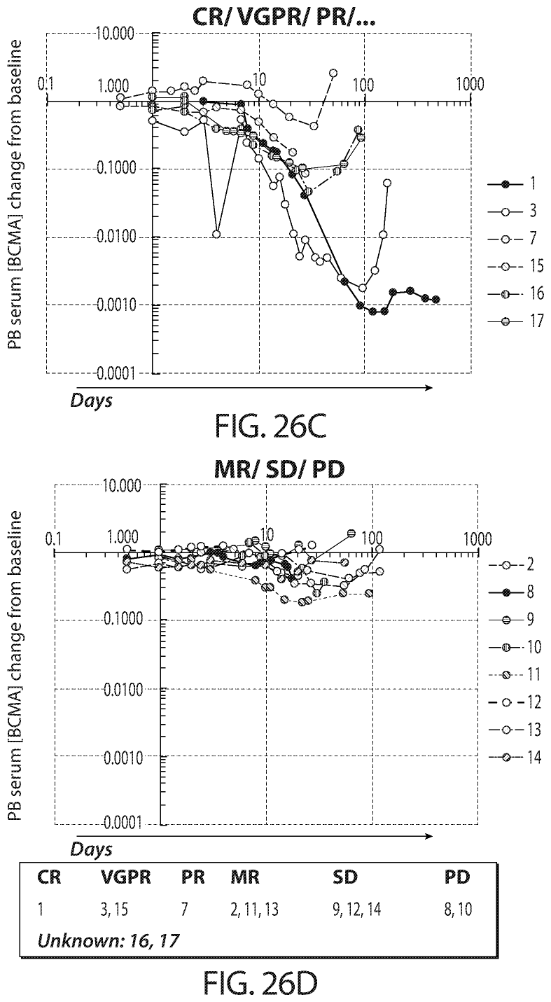

The serum level of BCMA was evaluated in 14 normal donors and 12 myeloma patients (FIGS. 25A and 25B). BCMA was present in normal donors at a serum concentration of about 40 ng/mL, and in myeloma patients at a median serum concentration of 176 ng/mL at baseline. Serum levels of BCMA were also acquired at various time points post infusion of CART-BCMA (FIGS. 26A, 26B, 26C, and 26D). These data demonstrate that some patients with high baseline serum BCMA levels responded well to CART-BCMA treatment (FIG. 26A). In addition, serum BCMA levels could serve as a marker of response to CART-BCMA treatment (FIGS. 26C and 26D).

### Evaluation of the Percentage of CD4+ and CD8+ CART Cells

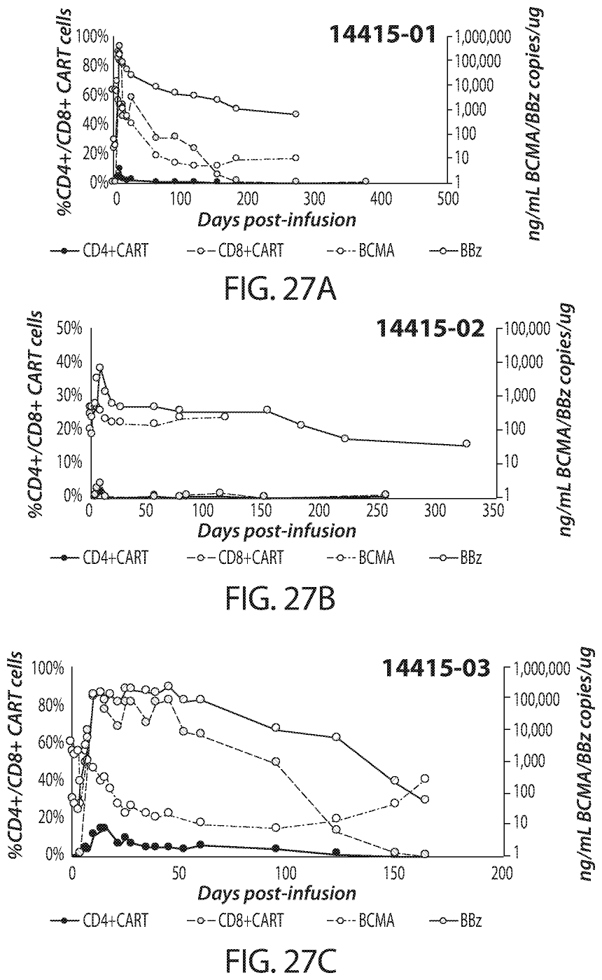

The percentage of CD4+ and CD8+ CART cells was acquired at various time points post infusion of CART-BCMA in three patients (FIGS. 27A, 27B, and 27C). Responders as shown in FIGS. 27A and 27C had a dramatic expansion of CAR T cells as seen by high BBz copy numbers. Expansion was mainly driven by CD8+ CARTs. Although non-responders also had high BCMA levels, no expansion was seen (FIG. 27B).

### Evaluation of CD4+ and CD8+ T Cell Subsets

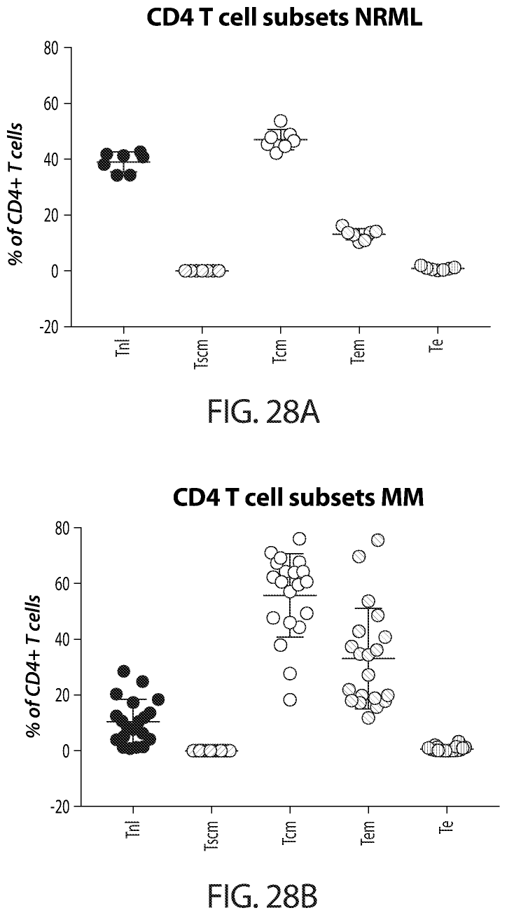

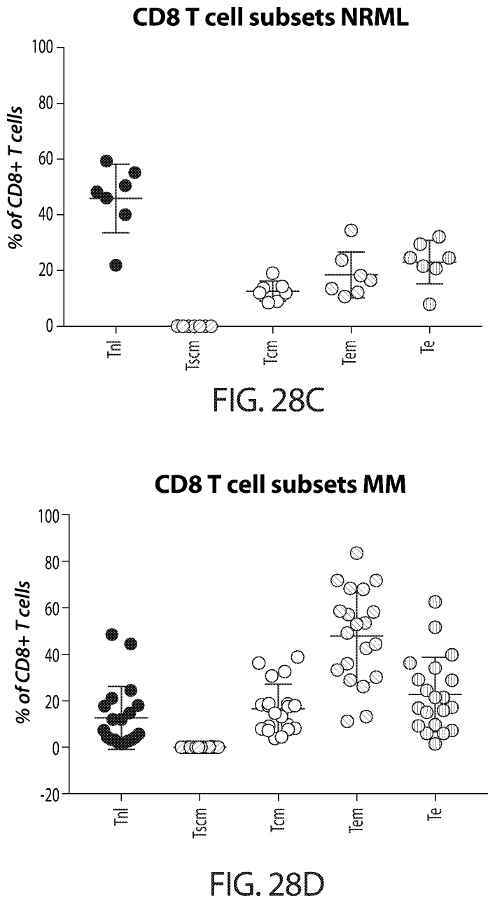

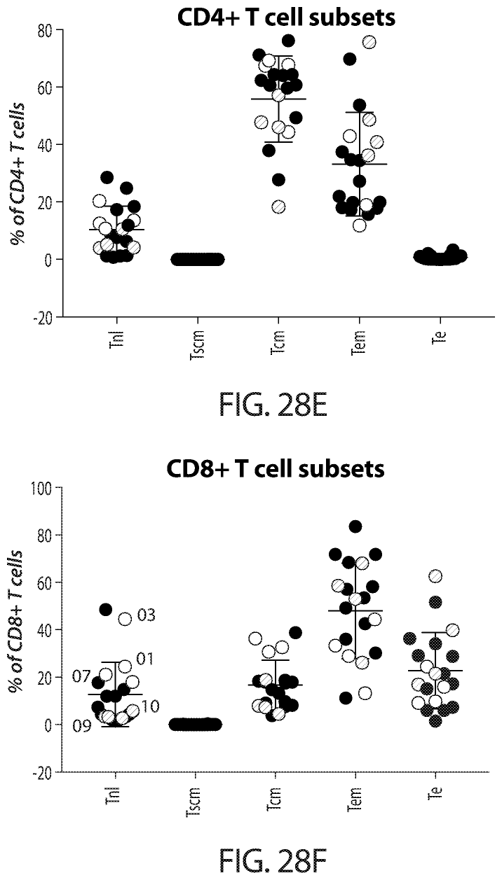

The level of CD4+ and CD8+ T cell subsets of normal donors and multiple myeloma (MM) patients were compared (FIGS. 28A, 28B, 28C, and 28D). MM patients had a lower percentage of Tn1 cells in comparison to normal donors. The level of Tscm and Te cells were similar in normal donors and MM patients. MM patients had a higher median percentage of Tem cells. The level of CD4+ and CD8+ T cell subsets in apheresis samples were also acquired from MM patients (FIGS. 28E and 28F).

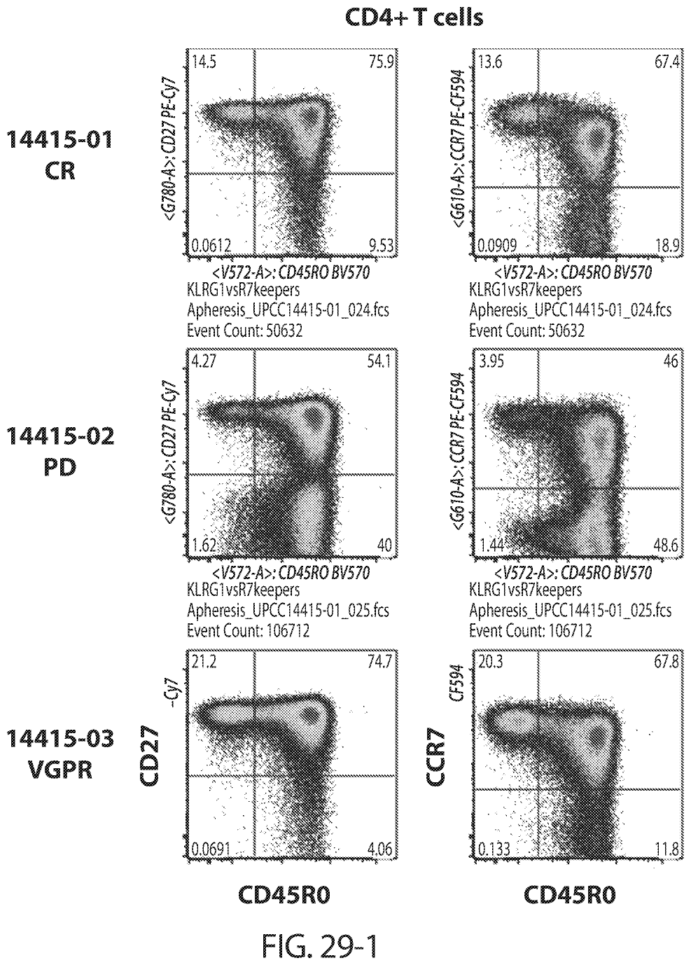

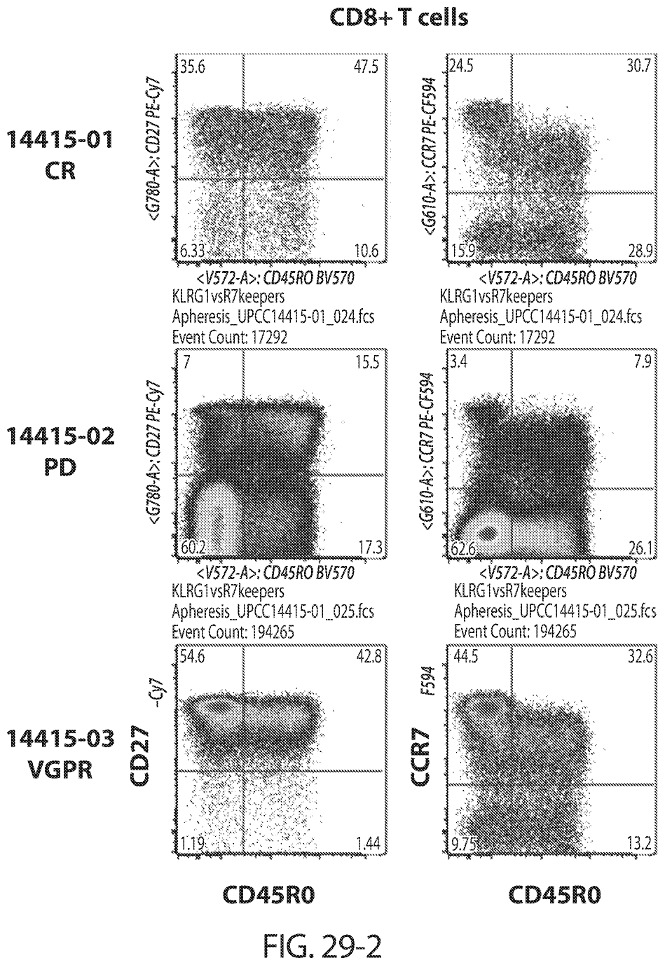

T cell differentiation in apheresis samples was acquired from MM patients (FIG. 29). The level of CD4+ and CD8+ T cell subsets (e.g., CD4+ or CD8+ cells based on the expression of PD1, CD27, and/or GzB) were also determined in MM patients (FIGS. 30A and 30B).

### Example 3: Predictive Correlates of Response to BCMA Chimeric Antigen Receptor (CAR) T Cell Therapy in Patients with Multiple Myeloma

This example describes studies that aimed to discover biomarkers that could predict the clinical response of subjects to treatment with CART-BCMA before the manufacturing of the CART-BCMA.

Peripheral blood samples were collected from a total of eight multiple myeloma (MM) human patients and were stained with a 14-parameter flow cytometry panel and analyzed on a flow cytometry instrument.

These MM patients received CART-BCMA treatment as described in Example 1. Based on clinical response at 28-days post infusion, patient samples were classified as Responders (R, defined as patients having CR, VGPR, or PR, N_{R}=3) or Non-Responders (NR, defined as patients having MR, SD, or PD, N_{NR}=5).

To identify a biomarker signature that correlated with clinical response, pre-gating was performed to limit analysis to viable, singlet, lymphocytes. Subsequently, pre-gated data was used for data mining and biomarker identification, using flowType (Aghaeepour et al. Bioinformatics. 28(7):1009-1016, 2012), a bio-statistical automated algorithm, based on R⁺ (https://www.r-project.org/about.html). FlowType uses a simple threshold or clustering algorithm to partition every channel/marker density to a positive and a negative cell population based on the assumption that the expression of a marker is either on or off (i.e., there are two distinct populations). These partitions are then combined to generate a set of multi-dimensional phenotypes. In addition, and to allow for exclusion of markers from subset identification, each marker could also be assigned a ‘neutral’ value (i.e., that marker is excluded from a phenotype). This algorithm generates a total number of possible phenotypes of 3^{N} where N is the number of markers (N=14 in the present experiment).

The length of a phenotype was limited to a maximum of four markers to allow for the identification of biologically meaningful phenotypes, as too many markers may lead to the generation of phenotypes with very small cell counts that would be difficult to interpret. This process generated a set of 1,697 possible phenotypes. Beyond these exploratory phenotypes, a subset of combinations of the above phenotypes, such as CD4:CD8 ratio, Viable T cells (%), % Monocytes, and % B-cells were also considered.

To measure the predictive power of each phenotype, a T-test was used to evaluate the difference between the measured phenotypes' cell frequencies (the number of cells in that phenotype divided by the total number of cells in the parent population) among Responders (R) and Non-Responders (NR). The most statistically significant phenotypes were selected for manual confirmation using FlowJo.

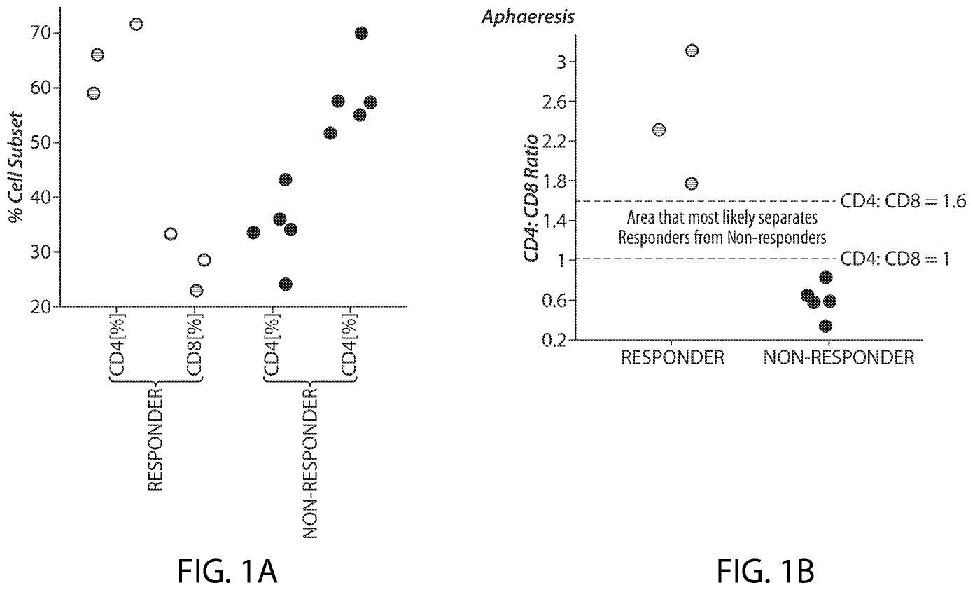

The CD4:CD8 ratio was found to be a clear differentiator in the aphaeresis samples, in terms of clinical response (FIGS. 1A and 1B). The area that most likely separates Responders from Non-Responders corresponds to a CD4:CD8 ratio range of between 1 and 1.6, suggesting that patients with higher levels of CD4 populations (e.g., a CD4:CD8 ratio of greater than or equal to 1.6) in their aphaeresis samples are likely to respond to the CART-BCMA treatment (FIG. 1B).

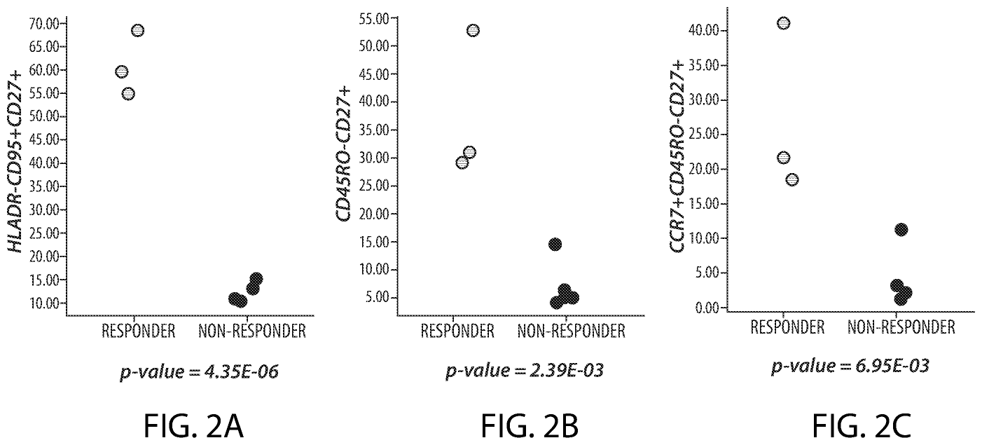

Higher levels of the CD8+ stem memory T cells (TSCM) populations HLADR-CD95+CD27+ (FIG. 2A), CD45RO−CD27+ (FIG. 2B), and CCR7+CD45RO−CD27+ (FIG. 2C) were also observed in Responders as compared to Non-Responders, suggesting that patients with higher levels of these TSCM populations in their aphaeresis samples are likely to respond to the CART-BCMA treatment.

The analysis, described above, of T cells in the pre-BCMA manufacturing apheresis samples from multiple myeloma patients demonstrated a high CD4:CD8 ratio in patients who later responded to the CART-BCMA therapy and a low CD4:CD8 ratio in Non-Responders. Thus, selecting for patients with a high CD4:CD8 ratio in their aphaeresis material may potentiate for a more efficacious treatment outcome. In addition, these data and the bioinformatics tools suggest that immunological biomarkers could be integrated as a means to identify which patients are most likely to respond to the CART-BCMA therapy, leading to an ideal personalized approach to cellular therapy.

### Example 4: Analysis of Multiple Myeloma Tumor Biopsies

Bone marrow core biopsy samples were acquired from patients enrolled in the University of Pennsylvania clinical trial entitled “Pilot Study Of Redirected Autologous T Cells Engineered To Contain an Anti-BCMA scFv Coupled To TCR And 4-1BB Signaling Domains in Patients With Relapsed and/or Refractory Multiple Myeloma” (NCT Number: NCT02546167; UPCC 14415) for analysis. Bone marrow core biopsy samples were collected prior to administration (“Pretreatment” or “Pre”) or on Day 28, Day 43, or Day 90 post-infusion. Biopsy samples were formalin fixed with Immunocal™ decalcification and embedded in paraffin; and sample processing quality was confirmed by in situ hybridization (ISH) to the house keeping gene PPIB RNA.

### Analysis of CD138+ MM Cell Localization and BCMA Expression

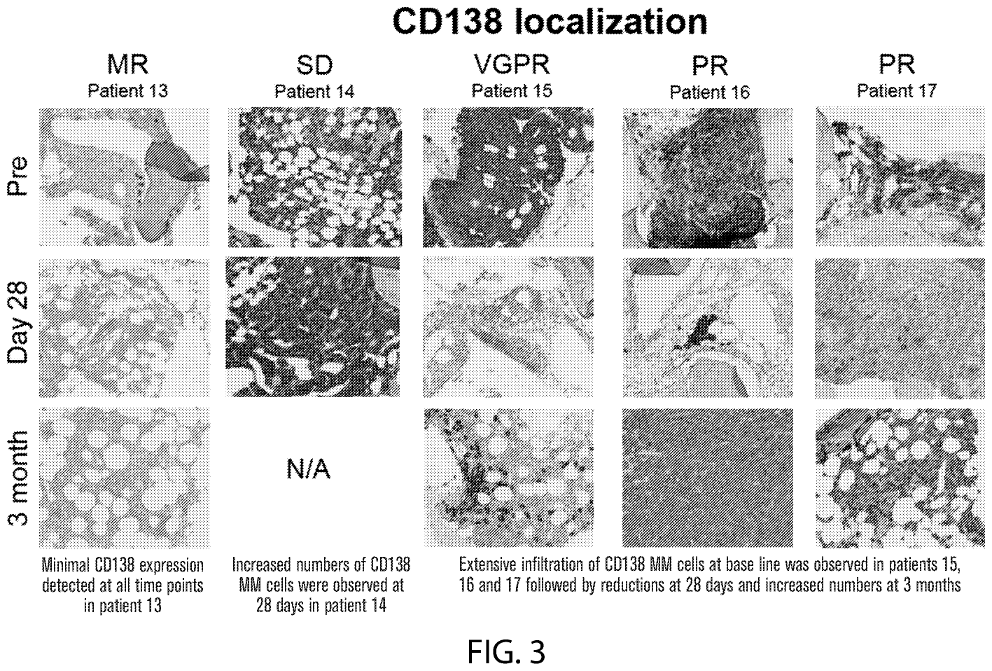

CD138+ MM cell localization data from bone marrow core biopsy samples from Patient 13, Patient 14, Patient 15, Patient 16, and patient 17 were acquired prior to administration (“Pretreatment” or “Pre”), and on Day 28 and Day 90 (“3 month”) post-infusion (FIG. 3). Patient 13 had minimal CD138 expression in their pretreatment, Day 28, and Day 90 samples (FIG. 3). Patient 14 had an increased number of CD138+ MM cells in their Day 28 sample compared to their pretreatment sample (FIG. 3). Patient 15, Patient 16, and Patient 17 each had extensive infiltration of CD138+ MM cells at baseline, followed by a reduction in the Day 28 samples and an increase in the 3-month samples (FIG. 3).

Patient outcomes to treatment with CART-BCMA and estimated % CD138 infiltration are provided in Table 29.

TABLE 29
Patient outcomes to CART-BCMA treatment and estimated % CD138 infiltration

| CD138 | | | | | | |
|---|---|---|---|---|---|---|
| staining | Estimated % CD138 infiltration | | | | | Pregression free |
| Patient | Pre | Response | Day 90 | D 43 | | survival (PFS) |
| Patient 7 | | | | 50% | Partial regression (PR) | 1.5 |
| Patient 8 | 70% | | | | Progressive disease (PD) | 0.5 |
| Patient 12 | | 40% | | | Stable disease (SD) | 1.5 |
| Patient 13 | 1% | 0% | 0% | | Minor response (MR) | 4+ |
| Patient 14 | 80% | 90% | | | Stable disease (SD) | 1.5 |
| Patient 15 | 95% | 5% | 10% | | Very good partial | |
| | | | | | regression (VGPR) | |
| Patient 16 | 50% | 5% | 95% | | Partial regression (PR) | 2+ |
| Patient 17 | 50% | 5% | 75% | | Partial regression (PR) | 2+ |

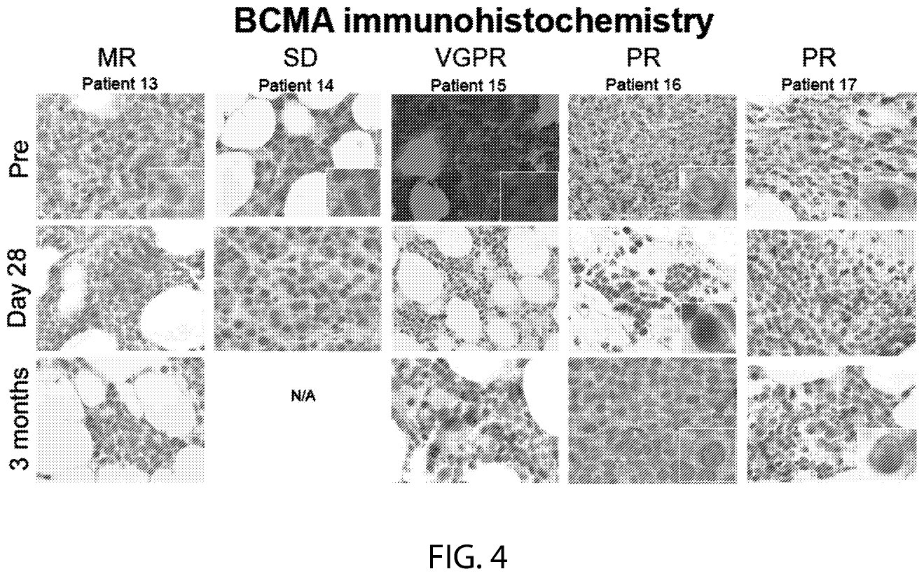

BCMA expression data from bone marrow core biopsy samples were similarly acquired and analyzed (FIG. 4).

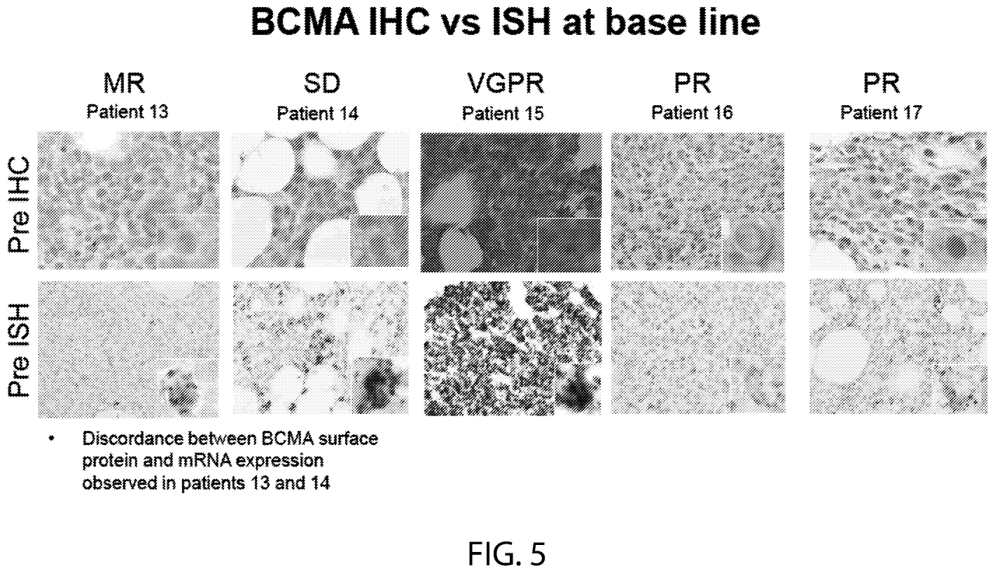

Discordance between BCMA protein expression as measured by IHC and BCMA mRNA expression as measured by ISH was observed in Patient 13 and Patient 14 (FIG. 5).

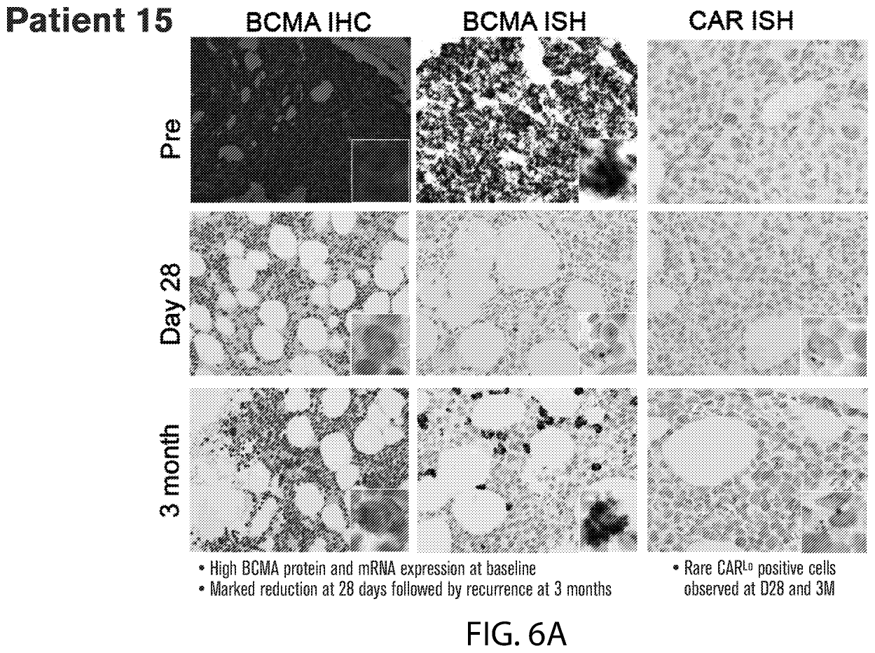

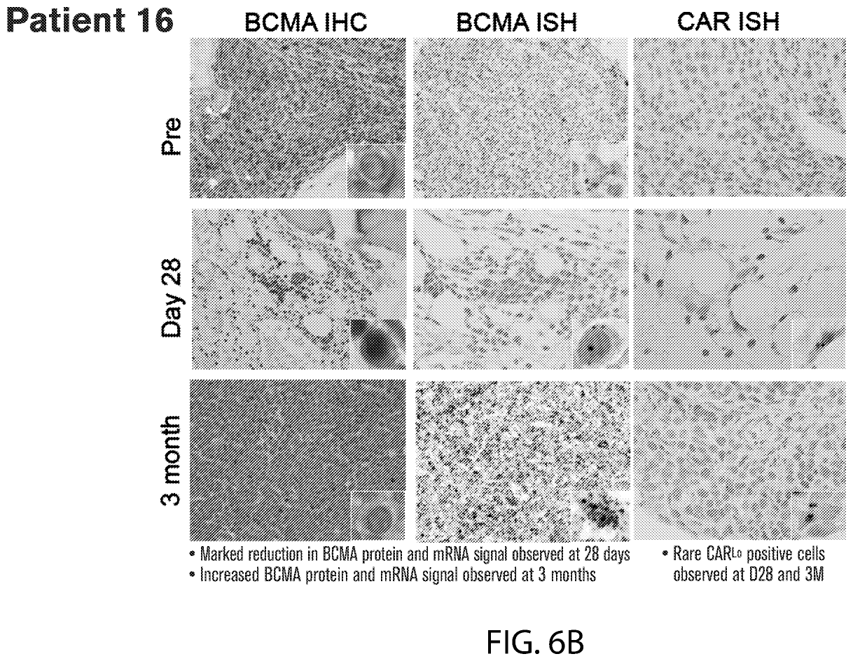

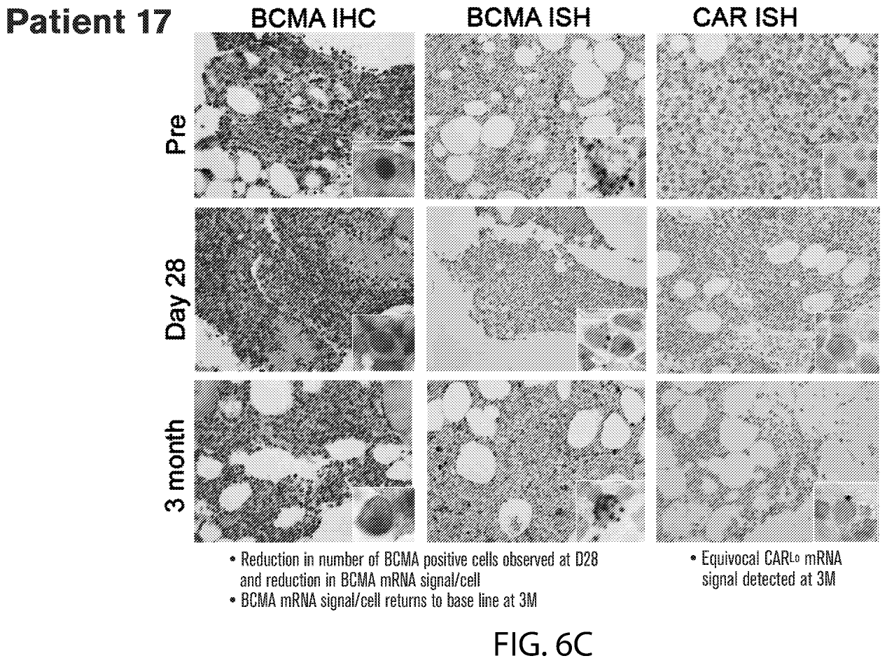

In Patient 15, high BCMA protein and mRNA levels were observed in pretreatment samples, followed by a marked reduction in Day 28 samples and a recurrence in Day 90 samples (FIG. 6A). Rare CAR^{Lo} positive cells were observed in Day 28 and Day 90 samples (FIG. 6A). In Patient 16, a marked reduction in BCMA protein and mRNA signal was observed at Day 28, followed by an increase at Day 90 (FIG. 6B). Rare CAR^{Lo} positive cells were observed at Day 28 and Day 90 (FIG. 6B). In Patient 17, a reduction in the number of BCMA positive cells and a reduction in BCMA mRNA signal/cell were observed at Day 28 (FIG. 6C). BCMA mRNA signal/cell returned to base line level at Day 90 (FIG. 6C). Equivocal CAR^{Lo} mRNA signal was detected at Day 90 (FIG. 6C).

In summary, variability in BCMA protein and mRNA expression was observed at base line and appeared to correlate with response in the sample set examined Decreased CD138 positive cell infiltration was observed in 3 of 5 patients at 28 days and was associated with decreased BCMA protein and mRNA expression. Increased CD138 positive cell infiltration was observed in these same patients at 3 months and was associated with a return of BCMA expression.

Analysis of IDO1, IFN-γ, and TGFβ mRNA Levels

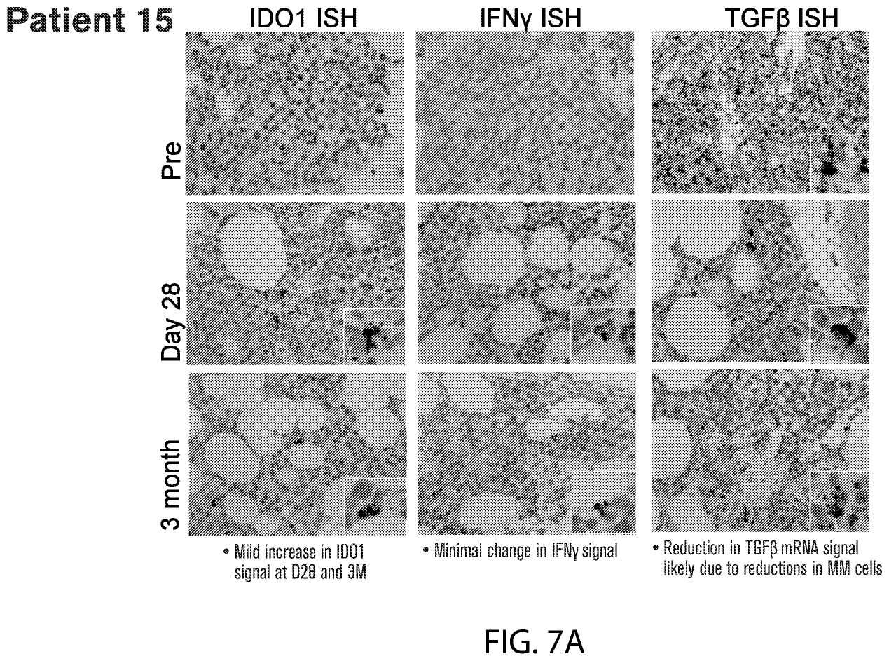

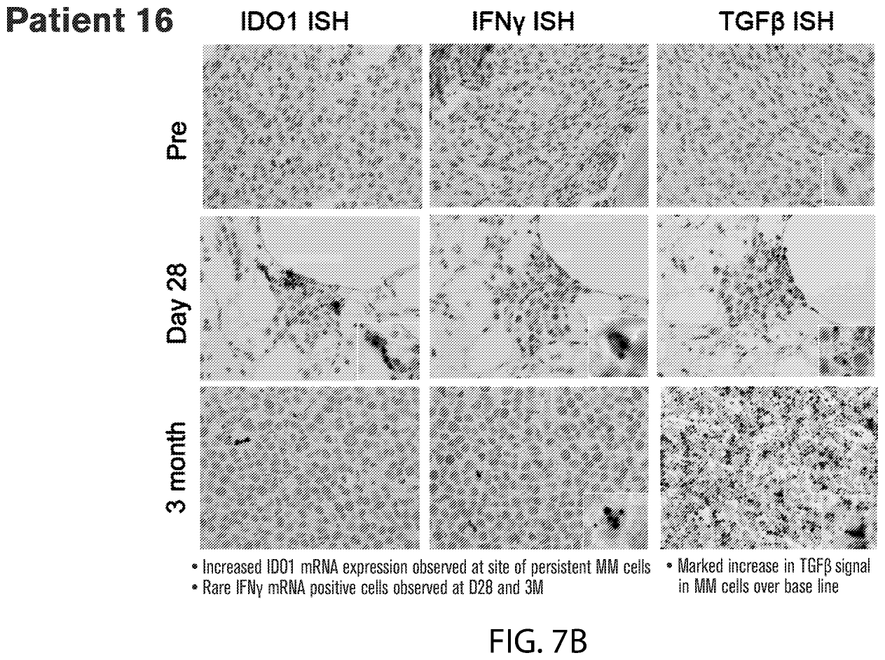

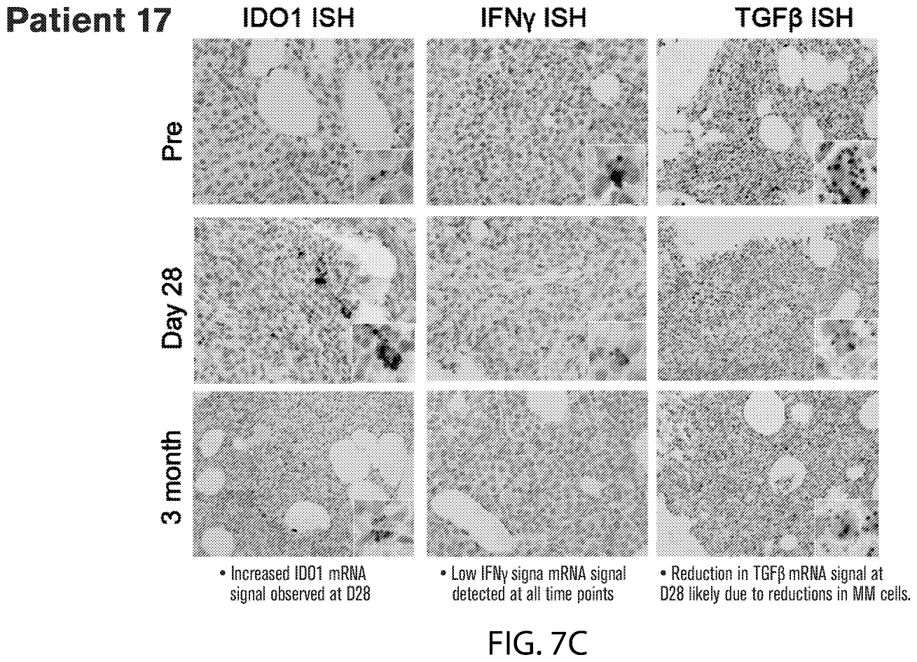

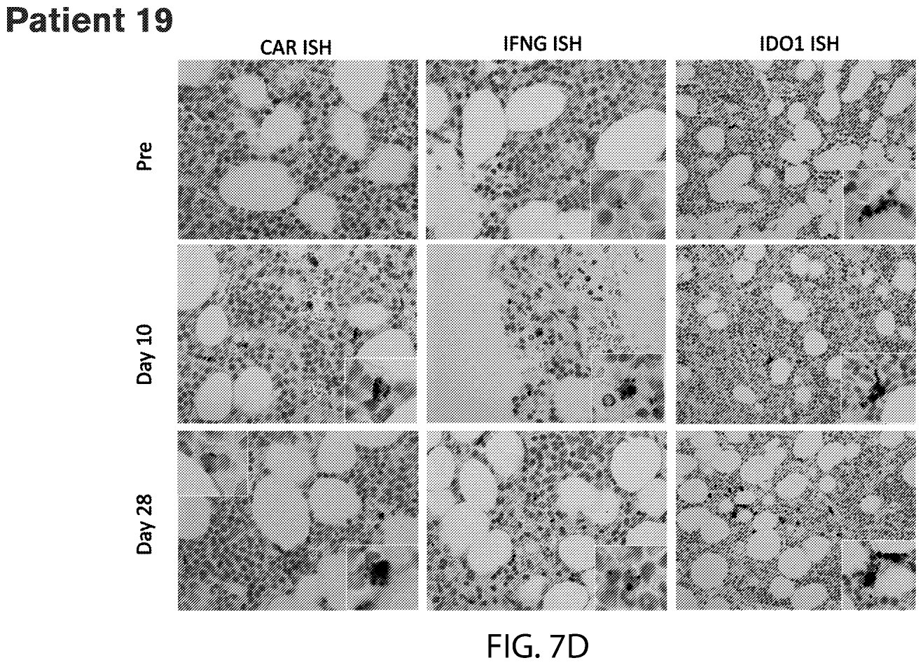

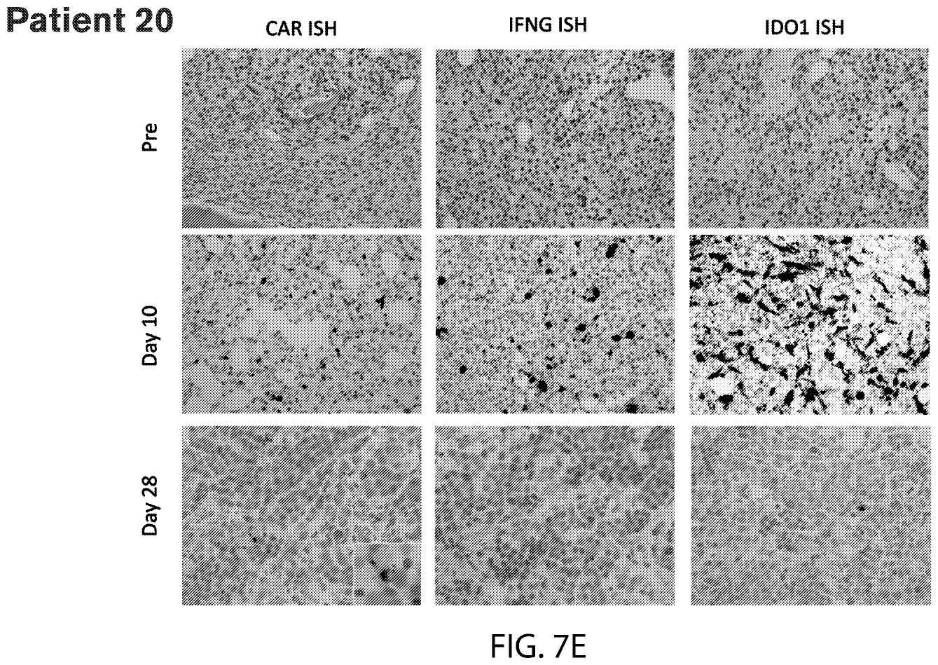

The expression of IDO1, IFN-γ, and TGFβ mRNA levels was determined by ISH in Pretreatment, Day 28, and Day 90 bone marrow core biopsy samples acquired from Patient 15 (FIG. 7A), Patient 16 (FIG. 7B), and Patient 17 (FIG. 7C).

In Patient 15, an increase in IDO1 mRNA levels was observed in the Day 28 and Day 90 samples as compared to the Pretreatment sample (FIG. 7A). Minimal change was observed in the level of IFN-γ mRNA in all tested samples (FIG. 7A). A reduction in TGFβ mRNA levels was observed in the Day 28 and Day 90 samples as compared to the Pretreatment sample, and this change may be due to a reduction in the level of MM cells (FIG. 7A).

In Patient 16, an increase in IDO1 mRNA levels was observed at the site of persistent MM cells (FIG. 7B). Rare IFN-γ mRNA positive cells were observed in the Day 28 and Day 90 samples, while a marked increase in TGFβ mRNA levels was observed in MM cells in the Day 90 sample over baseline (FIG. 7B).

In Patient 17, an increase in IDO1 mRNA levels was observed in the Day 28 sample as compared to the Pretreatment sample (FIG. 7C). A low level of IFN-γ mRNA was observed at all time points (FIG. 7C). A reduction in TGFβ mRNA levels was observed in the Day 28 sample as compared to the Pre sample, and this change may be due to a reduction in the level of MM cells (FIG. 7C).

These data show that a modest increase in IDO1 mRNA expression was observed in bone marrow at Day 28.

A similar ISH analysis was conducted in Pretreatment, Day 10, and Day 28 biopsy samples acquired from Patient 19 (FIG. 7D) and Patient 20 (FIG. 7E). An increase in IFN-γ and IDO1 mRNA expression was observed at Day 10.

### Analysis of PD-L1, PD1, CD3, and FoxP3 Protein Expression Levels

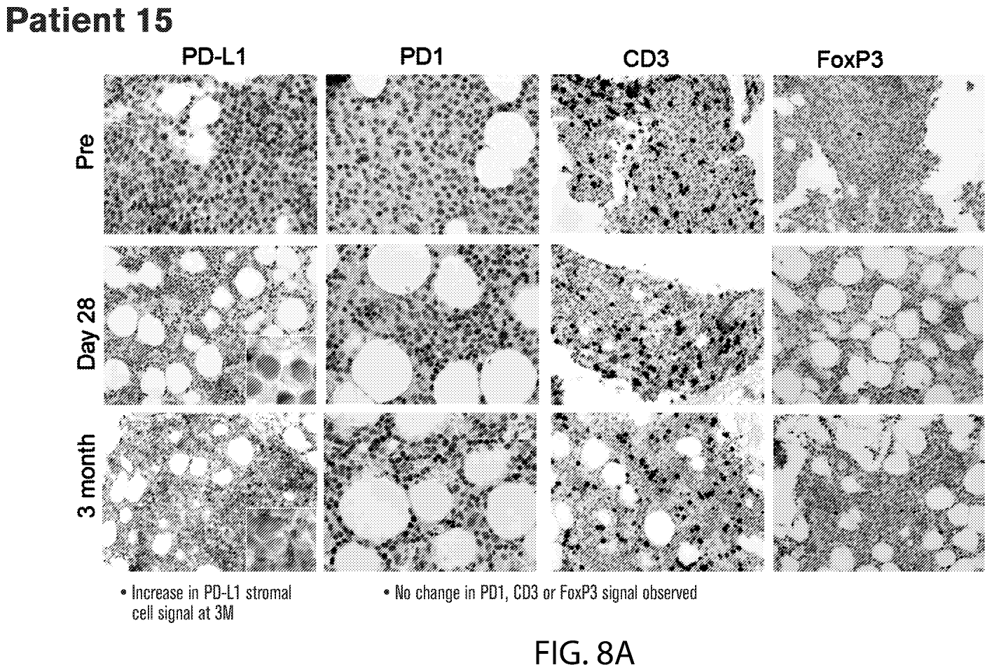

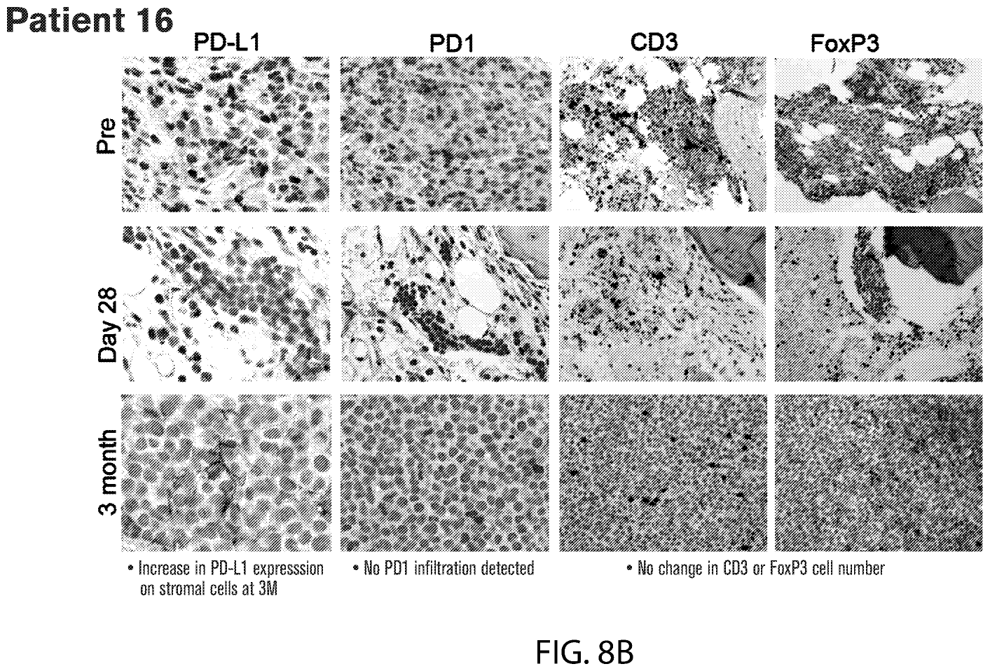

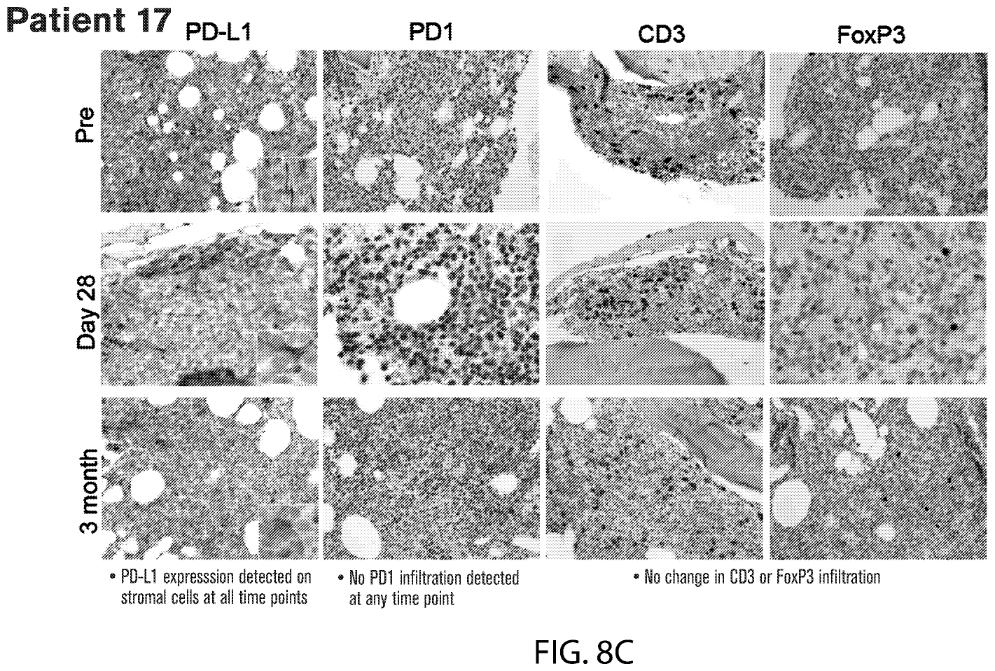

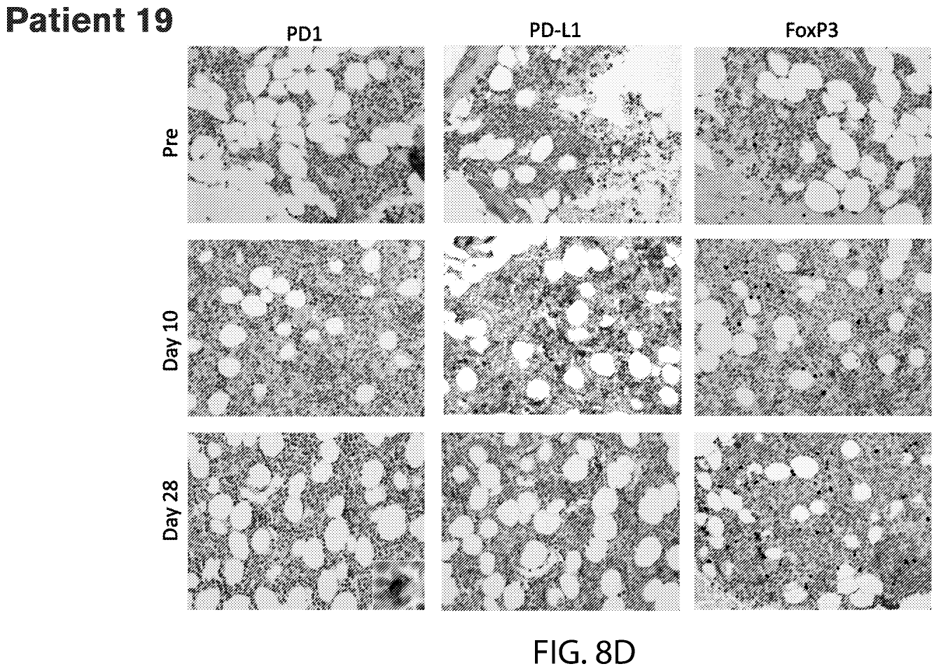

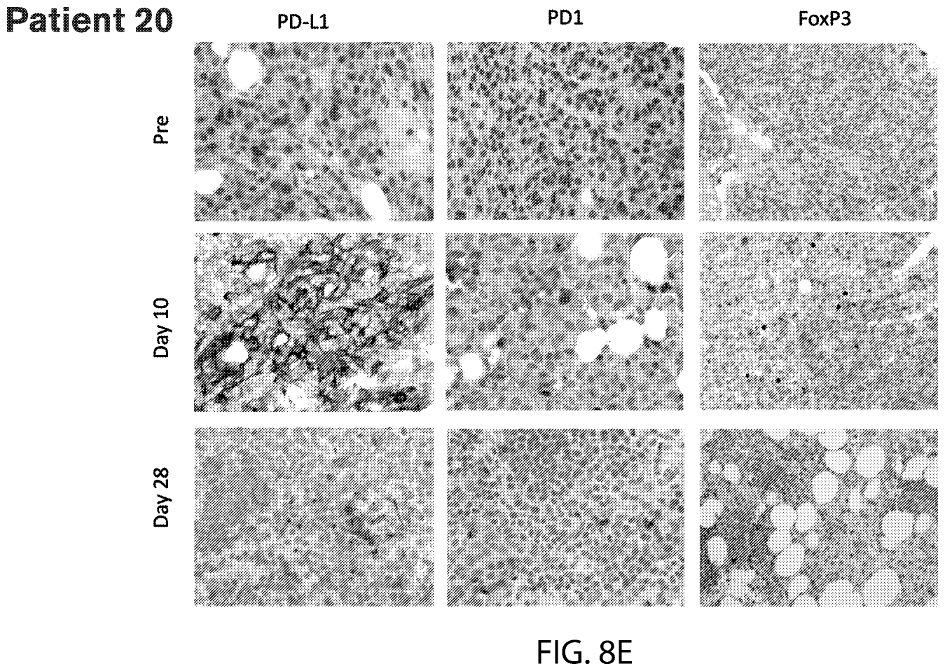

The level of PD-L1, PD1, CD3, and FoxP3 protein expression was determined by IHC in Pretreatment, Day 28, and Day 90 bone marrow core biopsies acquired from Patient 15 (FIG. 8A), Patient 16 (FIG. 8B), and Patient 17 (FIG. 8C).

In Patient 15, an increase in PD-L1 stromal cell expression was observed at Day 90 (FIG. 8A). No change in PD1, CD3, or FoxP3 expression was observed in the tested samples (FIG. 8A).

In Patient 16, an increase in PD-L1 stromal cell expression was observed at Day 90 (FIG. 8B). No PD1+ cell infiltration was detected (FIG. 8B). No change in CD3+ or FoxP3+ cell number was detected (FIG. 8B).

In Patient 17, PD-L1 stromal cell expression was observed at all time points (FIG. 8C). No PD1+ cell infiltration was detected and no change in CD3 or FoxP3 expression was observed in the tested samples (FIG. 8C).

A similar IHC analysis was conducted in Pretreatment, Day 10, and Day 28 biopsy samples acquired from Patient 19 (FIG. 8D) and Patient 20 (FIG. 8E). These two patients showed an increase in PD1 or PD-L1 in Day 10 samples.

These data show that while consistent changes in PD-L1, PD1, CD3, and FoxP3 were not observed, up-regulation of PD-L1 and/or PD1 in some patients may represent a potential escape mechanism.

### Analysis of CD19 Protein Expression Levels

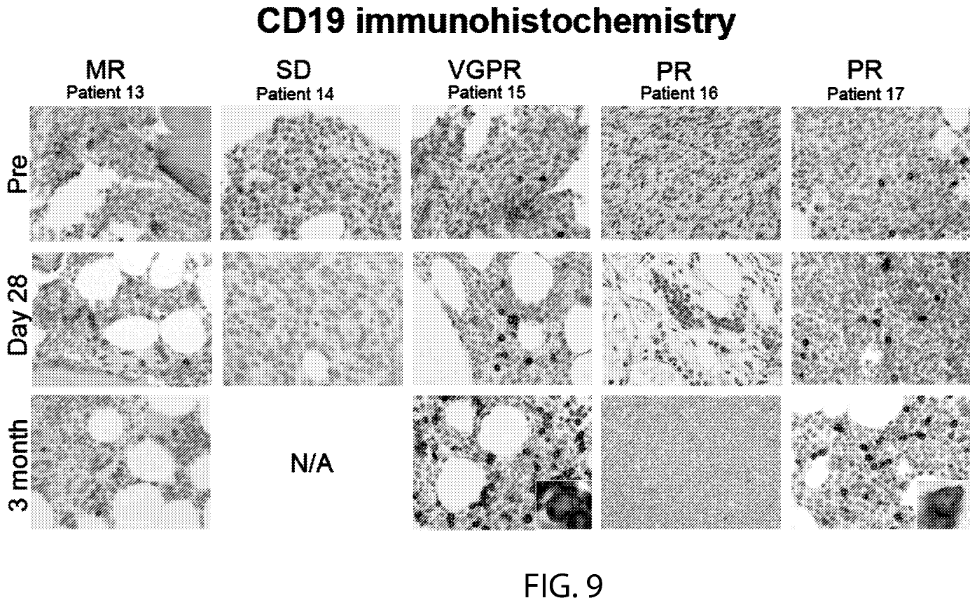

CD19 protein expression was determined by IHC in Pretreatment, Day 28, and Day 90 bone marrow core biopsies acquired from Patient 13, Patient 14, Patient 15, Patient 16, and Patient 17 (FIG. 9). An increase in the relative proportion of CD19 positive MM cells was observed in Patient 15 and Patient 17 at 28 days and 3 months (FIG. 9).

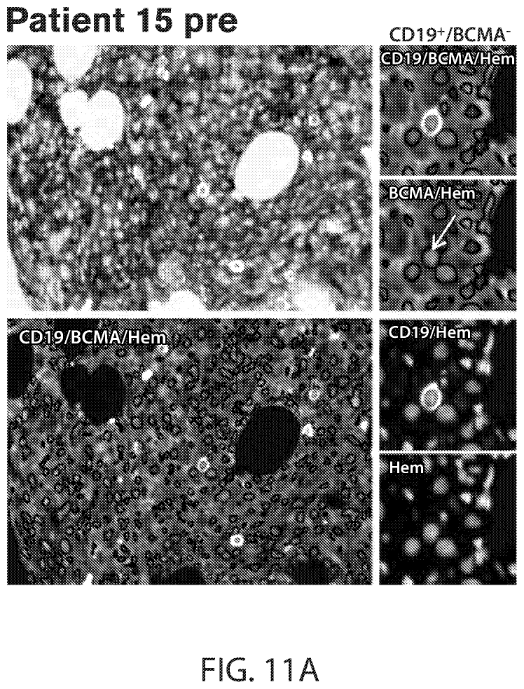

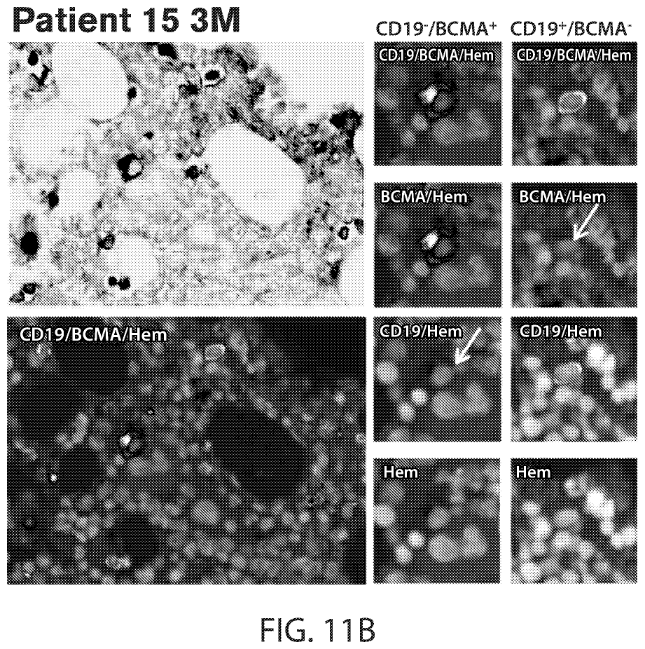

BCMA positive cells and CD19 positive cells were determined to be separate populations in the Pretreatment and Day 90 bone marrow core biopsies acquired from Patient 15 (FIGS. 11A and 11B).

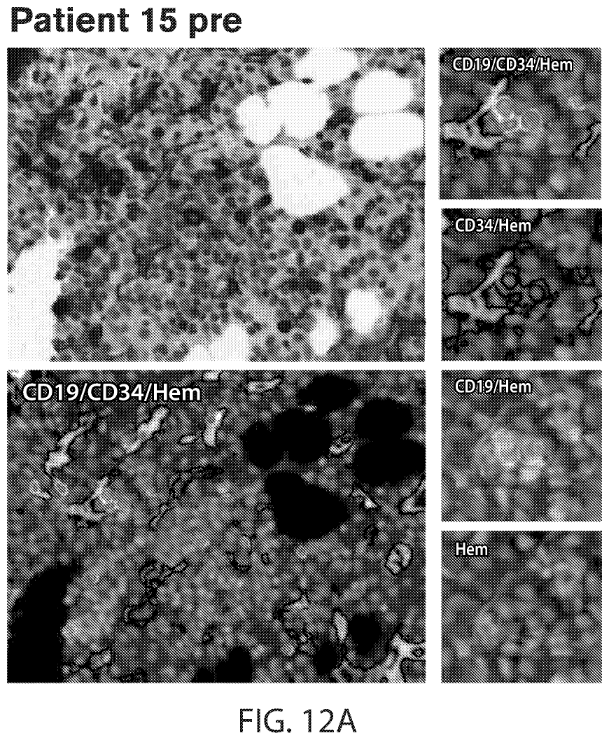

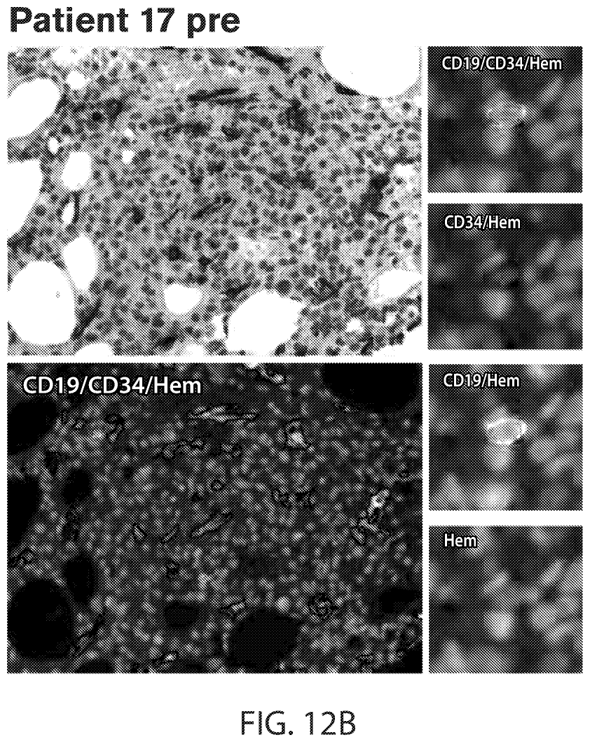

CD19+CD34^{dim} cell population was present in the Pretreatment bone marrow core biopsies acquired from Patient 15 (FIG. 12A) and Patient 17 (FIG. 12B).

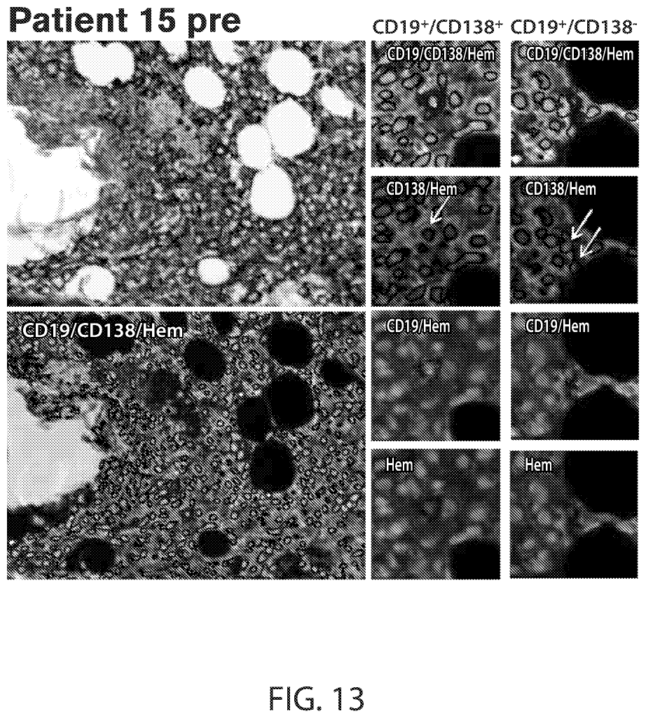

The CD19 population is variably CD138+ and CD138− in the Pretreatment samples acquired from Patient 15 (FIG. 13).

These data suggests that a combination therapy including a CART-BCMA and a CD19-targeted therapy may be beneficial in the treatment of MM patients.

### Analysis of CD20 Protein Expression Levels

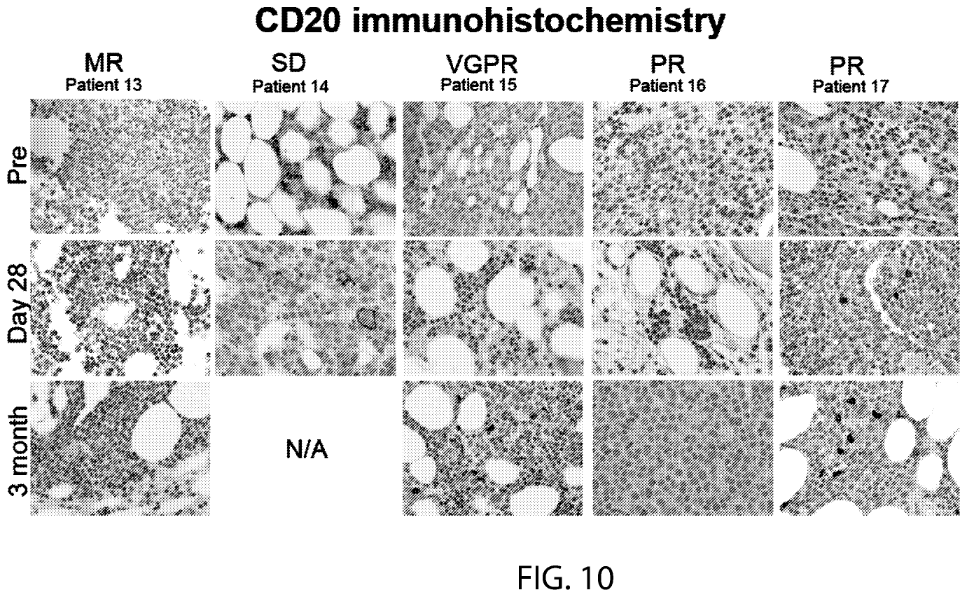

CD20 protein expression was determined by IHC in bone marrow core biopsies acquired prior to administration, and on Day 28 and Day 90 post-infusion of CART-BCMA from Patient 13, Patient 14, Patient 15, Patient 16, and Patient 17 (FIG. 10). Pre-existing CD20 positive MM cells were observed in samples acquired from patient 14 (FIG. 10). An emergence of CD20 positive MM cells was observed in patient 15 and patient 17 (FIG. 10).

These data suggest that a combination therapy including a CART-BCMA and a CD20-targeted therapy may be beneficial in the treatment of MM patients.

### Example 5: Clinical and Biologic Activity of B-Cell Maturation Antigen-Specific Chimeric Antigen Receptor T Cells (CART-BCMA) in Refractory Multiple Myeloma

### SUMMARY

Chimeric antigen receptor (CAR) T cells are emerging as a promising new therapy in hematologic malignancies. B-cell maturation antigen (BCMA) is a cell-surface receptor with expression largely restricted to plasma cells, making it a rational target for multiple myeloma (MM) therapy. A phase I study of autologous T cells transduced with a novel, fully human, BCMA-specific CAR containing CD3 and 4-1BB signaling domains (CART-BCMA) was conducted in subjects with relapsed/refractory MM after 3 or more prior lines of therapy. Reported here are mature results from Cohort 1 of this study, using a dose of 1-5×10⁸ CART-BCMA cells administered without prior chemotherapy conditioning. Nine subjects with a median of 9 prior therapy lines were treated; all had high-risk cytogenetics. CAR T cells were successfully manufactured and were detectable post-infusion in all cases. Four subjects (44%) had an objective response (1 PR, 2 VGPR, 1 sCR), with median duration of response of 4 months, including 1 subject with ongoing stringent complete response 21 months after CART-BCMA treatment. Compared with non-responders, responders had greater magnitude of in vivo CART-BCMA expansion, which was in turn associated with pre-manufacturing CD4:CD8 ratio and magnitude of expansion during manufacturing. Cytokine release syndrome was the most frequent treatment-related adverse event, occurring in 8 of 9 subjects (3 grade 3/4). Grade 4 encephalopathy was observed in 2 subjects. Median overall survival is estimated at 551 days. CART-BCMA infusions given without lymphodepleting chemotherapy are clinically active in heavily-pretreated MM patients and represent a novel approach to MM therapy.

### Results

### Protocol Design and Enrolment

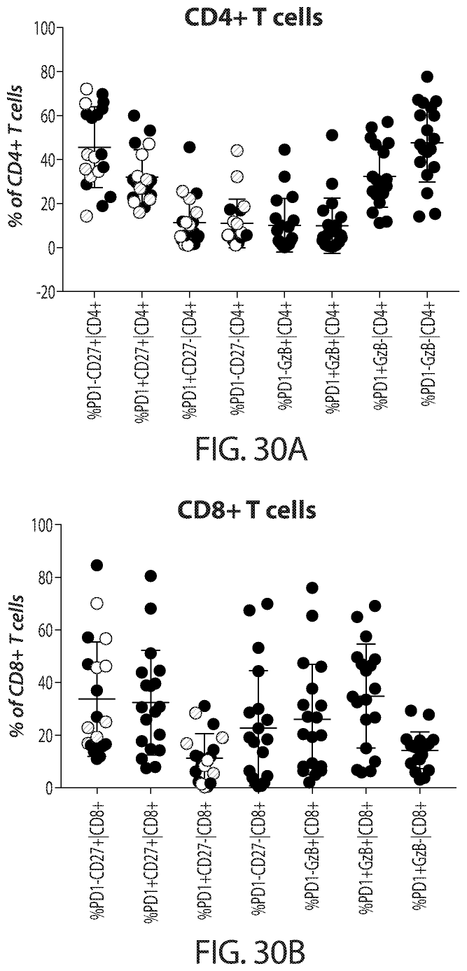

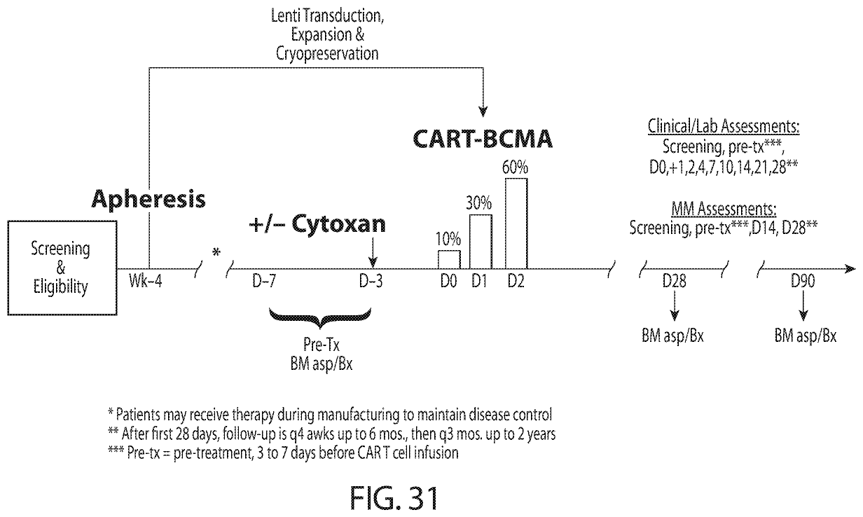

A phase I study (NCT02546167) was opened to evaluate the feasibility, safety, clinical activity, and biologic activity of manufacturing and administering CART-BCMA cells to relapsed/refractory myeloma patients. BCMA expression on myeloma cells was assessed by flow cytometry but no pre-specified level was required for enrolment. After a 2-week washout from therapy, subjects underwent steady-state leukapheresis to collect T cells for CART-BCMA manufacturing, typically a 3 to 4-week process. Anti-myeloma therapy could resume during manufacturing until 2 weeks prior to first CART-BCMA infusion. CART-BCMA cells were administered in an outpatient research unit over 3 days as split-dose intravenous infusions (10% of dose given on day 0; 30% on day 1, and 60% on day 2), as per prior adult CTL019 trials (Porter et al., Sci. Transl. Med. 7, 303ra139 (2015)). In cohorts 2 and 3 (described below), cyclophosphamide (Cy) was administered for lymphodepletion 3 days prior to first CART-BCMA infusion (FIG. 31).

Initially a standard 3+3 dose-escalation design was used, exploring 3 sequential cohorts: 1) 1-5×10⁸ CART-BCMA cells alone; 2) Cy 1.5 g/m²+1-5×10⁷ CART-BCMA cells; and 3) Cy 1.5 g/m²+1-5×10⁸ CART-BCMA cells. The protocol was later amended to allow treatment of additional subjects in each cohort, in order to gain more information about the safety and efficacy of CART-BCMA cells both with and without lymphodepleting conditioning (i.e. Cy), and at a higher (1-5×10⁸) and lower (1-5×10⁷) dose. Reported here are outcomes for the 9 subjects treated on Cohort 1 with CART-BCMA cells alone, now with mature follow-up. Enrollment and follow-up for Cohorts 2 and 3 are ongoing.

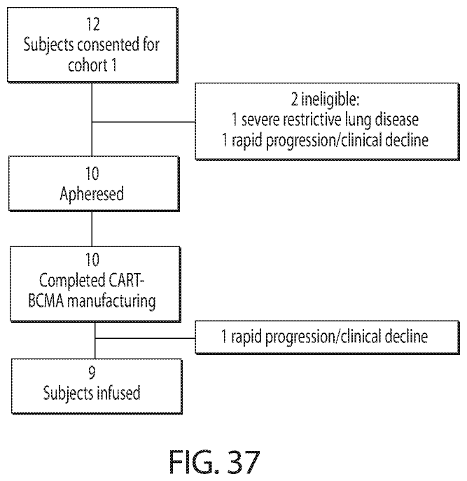

Twelve subjects consented during Cohort 1 enrollment; 2 never collected T cells (1 ineligible due to severe restrictive lung disease; 1 with rapid disease progression/clinical decline). Ten successfully manufactured CART-BCMA cells; 1 was never infused due to rapid progression/clinical decline, and 9 were infused between November 2015 and September 2016 (FIG. 37).

### Subject and CART-BCMA Product Characteristics

Subject demographics, prior lines of therapy, and disease characteristics are summarized in Table 30, with individual details shown in Table 32. Median age of treated subjects was 57, with 67% male. These subjects had a median of 9 prior lines of therapy, and 8/9 (89%) were dual-refractory to at least 1 proteasome inhibitor and immunomodulatory agent. All had at least 1 high-risk cytogenetic abnormality; 67% had either deletion 17p or a TP53 mutation. Baseline tumor burden was high (median 80% myeloma cells on pre-treatment bone marrow biopsy), and 2/9 (22%) had extramedullary disease. Median absolute lymphocyte count (ALC) and total CD3 count pre-leukapheresis were 830 and 325 cells/tit, respectively, and had declined to 500 and 258 cells/tit, respectively, by the time of CART-BCMA infusions, reflecting the advanced disease and extensive prior treatment in this cohort.

All 9 subjects successfully manufactured the minimum goal CART-BCMA cells (1×10⁸), though 1 subject required 2 leukapheresis/manufacturing attempts. Median transduction efficiency was 22.2% (range 9.6-33.3%), with median 12.7-fold expansion of seeded cells during manufacturing. Final products were comprised of a median of 96% CD3+ T cells, with median CD4/CD8 ratio of 1.6. Six subjects received all 3 planned CART-BCMA infusions, with 3 (subjects 01, 03, and 15) receiving only 40% of planned dose (3′ infusion held due to fevers and signs of CRS). Further details of manufacturing, product characteristics, and dosing for each subject are shown in Table 33.

### Clinical Outcomes

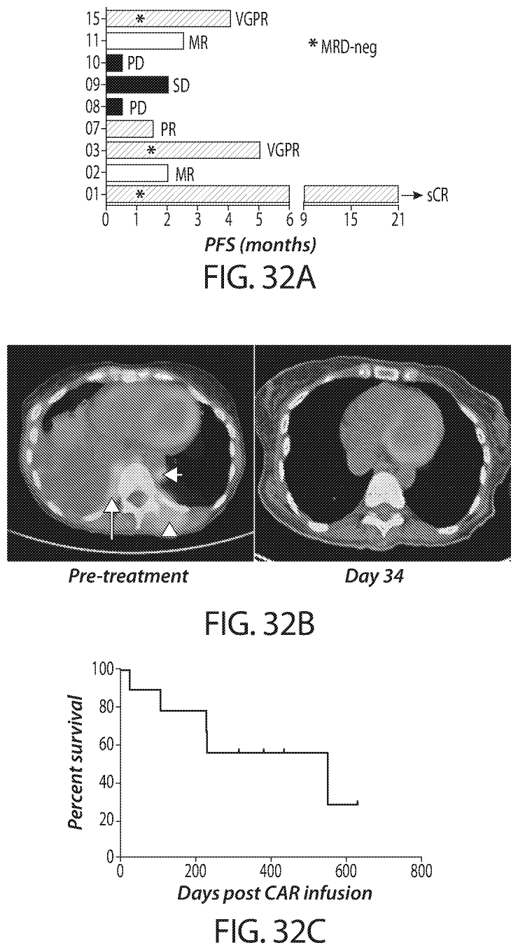

Four of 9 subjects (44%) achieved a partial response (PR) or better, including 1 PR, 2 very good partial responses (VGPR), and 1 stringent complete response (sCR). Two additional subjects had minimal response (MR), and 3 had no response (FIG. 32A). Median time to first response was 14 days. Based on Kaplan-Meier estimates, median duration of response (for those with PR or better) was 120 days (range 29-665+); and median progression-free survival (PFS) was 65 days (range 13-679+). Three subjects had no detectable myeloma by flow cytometry (estimated sensitivity 10⁻⁵) from marrow aspirates performed on day 28 (subjects 01, 15) or day 45 (subject 03) after CART-BCMA infusions. Subjects 03 and 15 achieved VGPR but progressed at 5 and 4 months, respectively. Subject 01 had 11 prior lines of therapy, was refractory to bortezomib, lenalidomide, carfilzomib and pomalidomide, and had deletion 17p along with mutations in TP53 and NRAS, reflecting very poor-risk disease. He was progressing rapidly prior to CART-BCMA therapy, with 70% marrow plasma cells, serum M-spike of 2.0 g/dL, 24-hour urine M-spike of 3900 mg, serum free kappa of 6794 mg/L, hypercalcemia and acute renal insufficiency (serum creatinine 1.82 mg/dL). He had gradual evolution of his response from PR (day 14) to VGPR (month 3), CR (month 6), and then to sCR (month 9), and remains in sCR 21 months after CART-BCMA infusion. One subject (03) with extramedullary involvement of paraspinal muscles and pleura had complete metabolic response on PET/CT 5 weeks after CART-BCMA infusion, including resolution of malignant pleural effusion (FIG. 32B), demonstrating ability of CART-BCMA cells to traffic outside blood and marrow compartments. The other subject (08) with extramedullary disease had no response. At time of data cutoff (9/11/17), 5 subjects had died, and estimated median overall survival (FIG. 32C) was 551 days (range 24-679+).

### Safety

Grade 3 or higher adverse events were seen in 8/9 (88%) of subjects and are summarized in Table 31, with all adverse events for each subject listed in Table 34. Cytokine release syndrome (CRS), a well-described complication of CAR T cell therapy, was observed in 8 subjects: 1 grade 1, 4 grade 2, 3 grade 3, and 1 grade 4. Median time of CRS onset was 3.5 days after first infusion (range 1-8), with a median duration of 8 days (range 3-12), and median duration of hospitalization of 9 days (range 3-40). CRS was associated with elevations in ferritin and C-reactive protein. Four subjects received tocilizumab, the anti-IL6 receptor antibody, with 3 requiring a second administration (subjects 01, 03, 08), and 2 requiring care in the intensive care unit.

Neurotoxicity is a common adverse event after CAR T cell therapy, ranging from mild confusion or concentration defects to focal neurologic deficits, global encephalopathy, aphasia, seizures, and/or obtundation. Subject 01 had grade 1 confusion, which resolved without intervention, in the setting of grade 3 CRS. Two subjects (03 and 08) developed severe (grade 4) neurotoxicity following CART-BCMA infusions. Both had high tumor burden with extramedullary disease and rapid progression at time of treatment, and both had received tocilizumab for severe CRS. Subject 03 developed obtundation and recurrent seizures requiring intubation, in the setting of rising peripheral lymphocyte count suggesting rapid CART-BCMA proliferation. MRI of the brain on day 15 showed diffuse white matter enhancement most pronounced in the posterior lobes, with sulcal effacement suggestive of early cerebral edema. At the time cerebral edema had not yet been described as a consequence of CAR T cell therapy (Abbasi et al., JAMA 317, 2271 (2017)), and her clinical and radiologic picture was felt to be most consistent with posterior reversible encephalopathy syndrome (PRES). She received high-dose intravenous steroids (methylprednisolone 1 g/day×3 days) without improvement, and then received cyclophosphamide 1.5 g/m2 on day 17, with rapid neurologic improvement within 48 hours, near complete resolution of abnormal enhancement on MRI day 23, and no residual neurologic deficits. Further details are described separately (Garfall et al., Blood 128, 5702-5702 (2016), incorporated by reference in its entirety).

Subject 08 had rapidly progressive myeloma at time of CART-BCMA infusions, with 80% marrow plasma cells and extramedullary involvement of skin, lymph nodes, liver, adrenals, and kidneys, along with progressive renal dysfunction (Cr 2.87 g/dL). He developed grade 4 CRS on day 15 associated with worsening kidney injury, atrial fibrillation, hypoxia, hypotension, coagulopathy, delirium, and transaminitis He had hemodynamic stabilization after tocilizumab, but had progressive grade 4 encephalopathy/obtundation requiring intubation day 17. MRI of the brain was unremarkable with no evidence of cerebral edema, but progressive myeloma in the base of skull was noted. He received steroids with improvement in mental status and was extubated on day 20. On day 22 he developed recurrent shock and hypoxia requiring intubation; blood cultures subsequently showed candidemia. Peripheral blood now showed 13% circulating plasma cells and labs confirmed progressive myeloma; in this setting his family opted for comfort care only and he expired on day 24. No other deaths occurred while on study.

### Kinetics of CART-BCMA Engraftment and Persistence

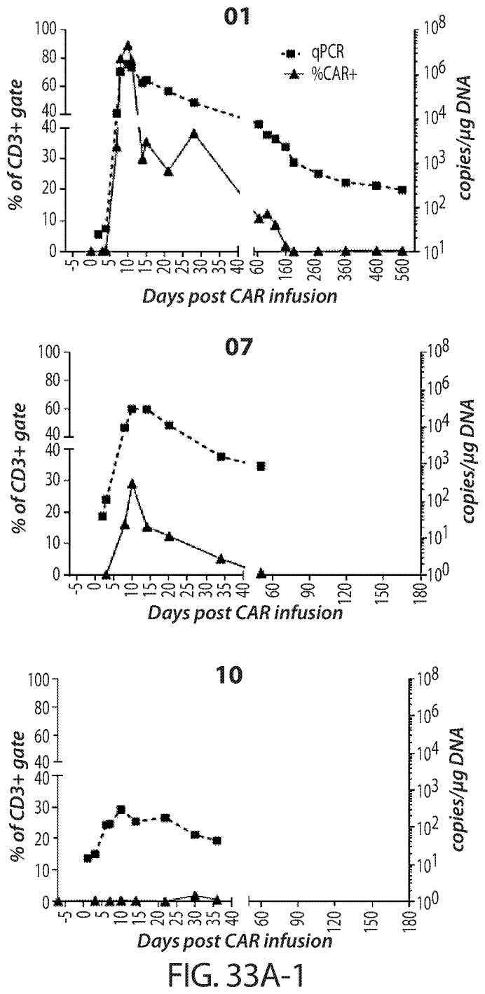

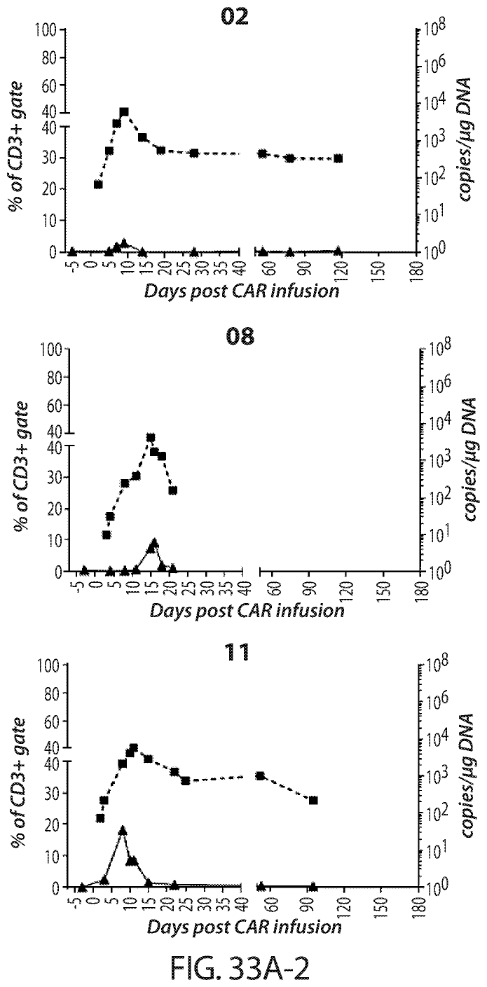

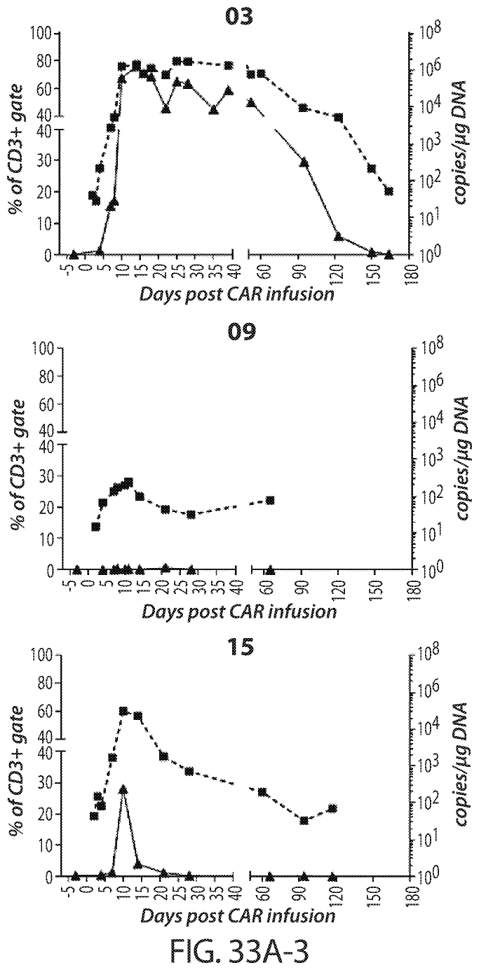

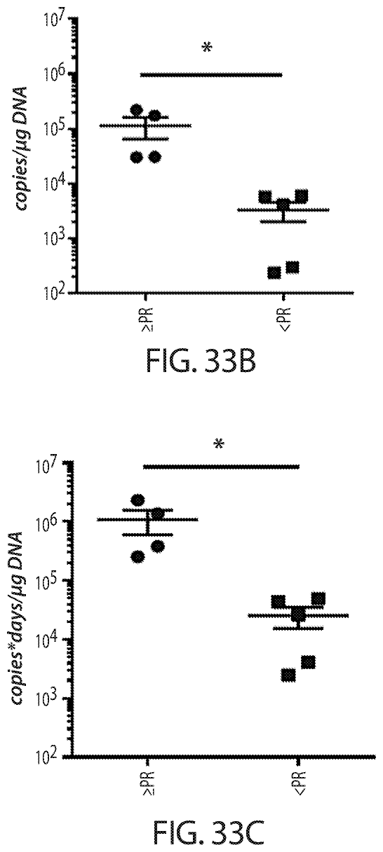

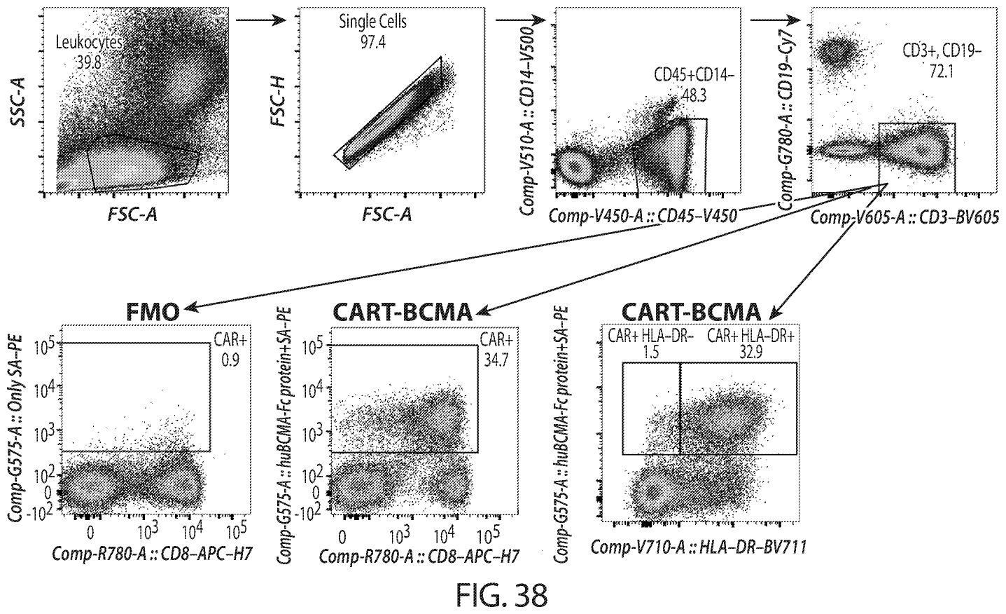

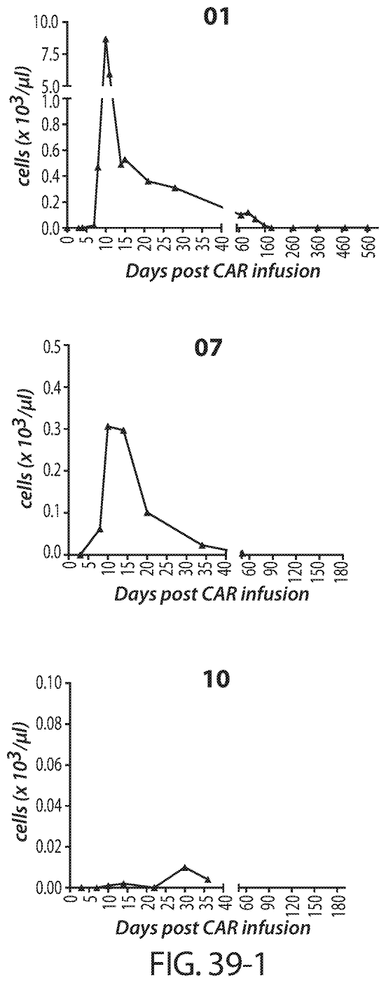

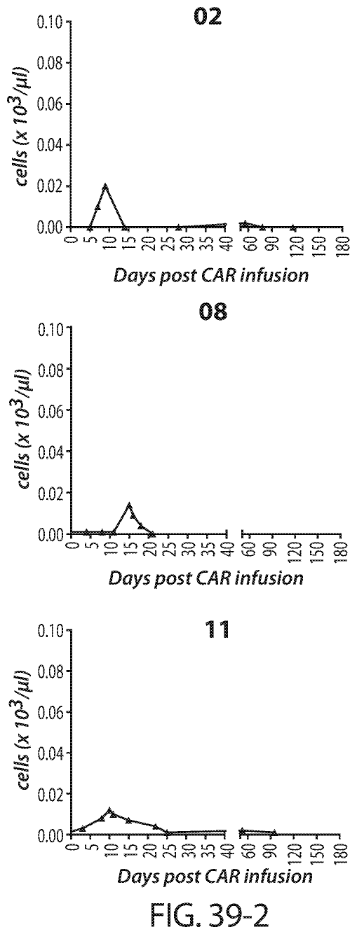

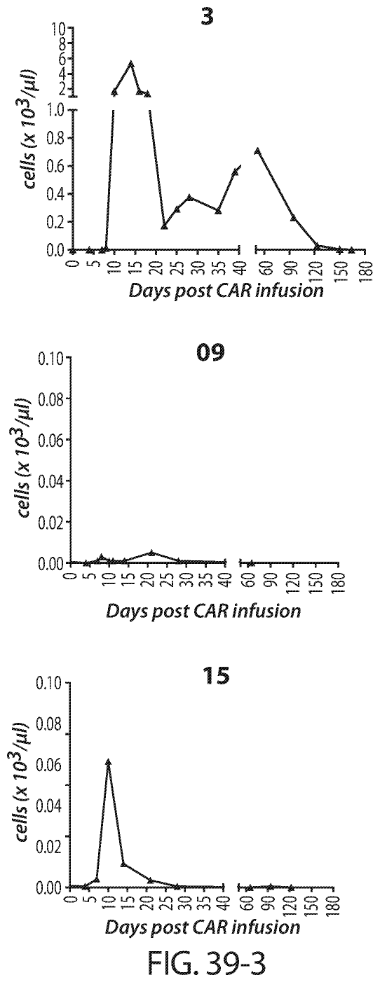

All infused subjects had detectable CART-BCMA cells in peripheral blood by qPCR analysis, and CAR⁺ T cells were detectable in 8/9 subjects by flow cytometry (FIG. 33A; FIG. 38 for representative staining). Expansion peaked at day 10 for most subjects, with CART-BCMA cells comprising over 75% of all circulating CD3+ T cells in the two subjects (01, 03) with greatest expansion, corresponding to 5300 and 8700 circulating CART-BCMA cells per μL of blood, respectively, at the peak of expansion (FIG. 39). Despite a predominance of CD4+ T cells in the CART-BCMA product pre-infusion, CART-BCMA cells circulating in blood were predominantly CD8+, and were highly activated, with a median of 94% (range 33-98%) of CAR+CD3+ cells expressing HLA-DR during peak expansion (Table 35). CART-BCMA levels in marrow aspirates generally mirrored those in peripheral blood, and also were elevated in pleural fluid and cerebrospinal fluid for subject 03 (Table 36). CART-BCMA cells showed a limited duration of detection within blood in most subjects. In all but 2 subjects (01, 03), CART-BCMA cells were no longer detectable by flow cytometry after day 28 but remained detectable by qPCR to day 60 in ⅞ tested, including in subject 01 (in stringent CR), who continues to have detectable cells at 21 months (FIG. 33A). Responses were significantly associated with peak expansion by qPCR (median 102507 copies/μg DNA for ≥PR vs. 4187 copies/μg for <PR, p=0.016), as well as with persistence over the first 28 days, as measured by the area under the curve (AUC_{0-28d}) (median 885181 copies*days/μg DNA for ≥PR vs. 26183 copies*days/μg DNA for <PR, p=0.016) (FIG. 33B).

### Changes in Soluble Factors Post-CART-BCMA

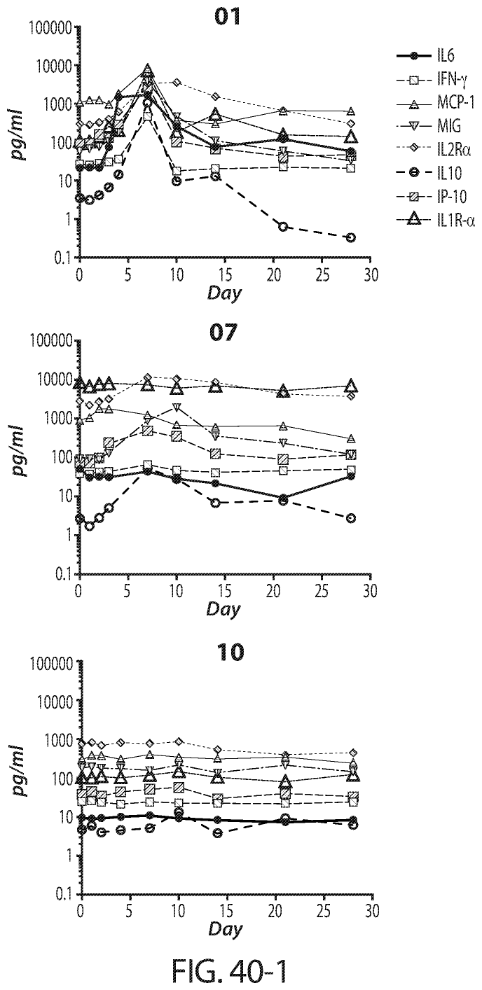

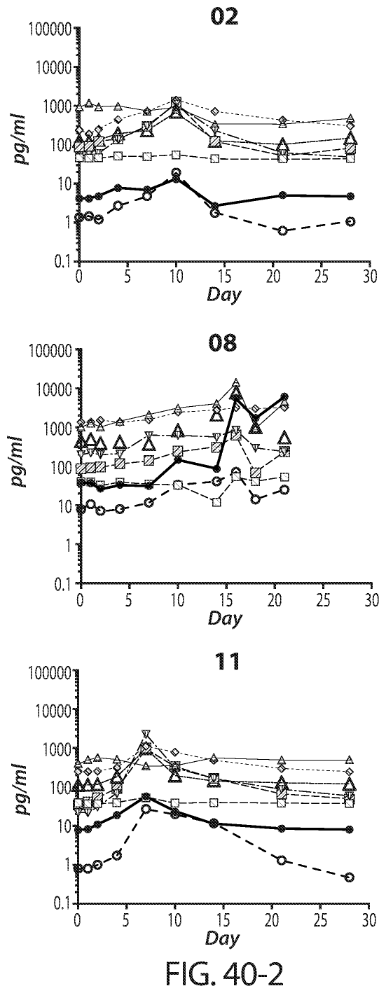

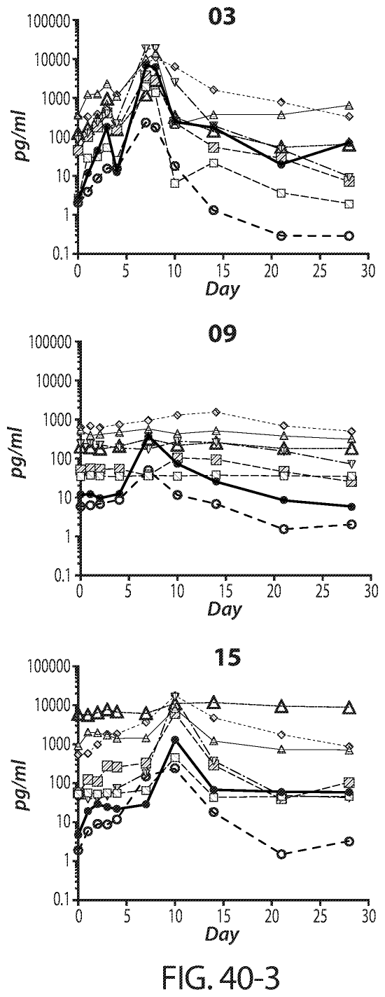

A total of 30 cytokines were quantified in peripheral blood serum before and after CART-BCMA infusion. The most consistent changes from baseline (>5-fold increase) were seen for IL-6, IL-10, monokine induced by interferon-gamma (MIG, CXCL9), IP10, and IL-1 receptor alpha (FIG. 40). Cytokine increases were most pronounced in subjects with greatest expansion and response, peaking at time of peak expansion and with clinical manifestations of cytokine release syndrome, similar to patterns previously described with CD19-directed CAR T cells (Porter et al., Sci. Transl. Med. 7, 303ra139 (2015); Teachey et al., Cancer Discov. 6, 664-679 (2016)). The subjects with the deepest responses (01, 03, 15) all had peak IL-6, IL-10, and MIG concentrations >50-fold above baseline. The timing of CRS also was associated with response, with a median onset of 2 days (range 1-3) after first infusion in subjects with PR or better, compared with 4.5 days (range 4-8) in subjects without PR (p=0.029, Mann-Whitney test).

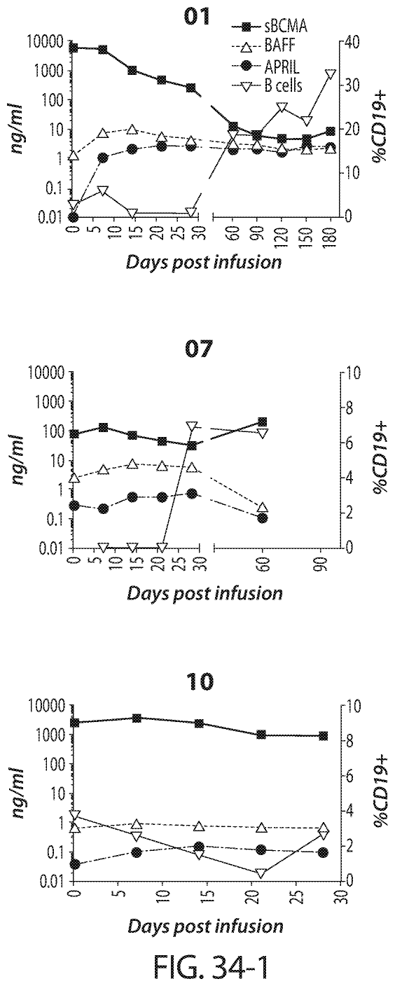

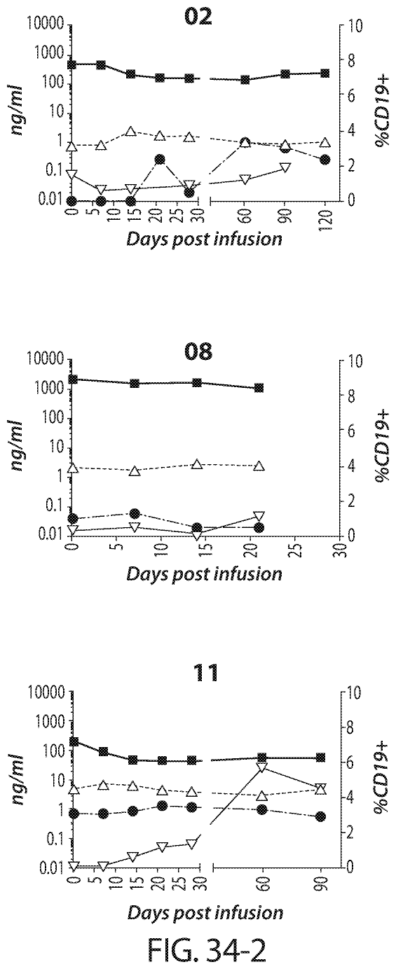

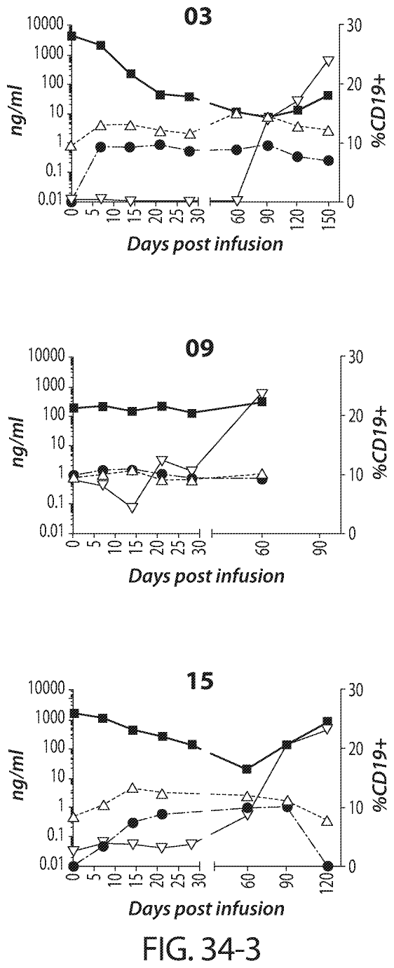

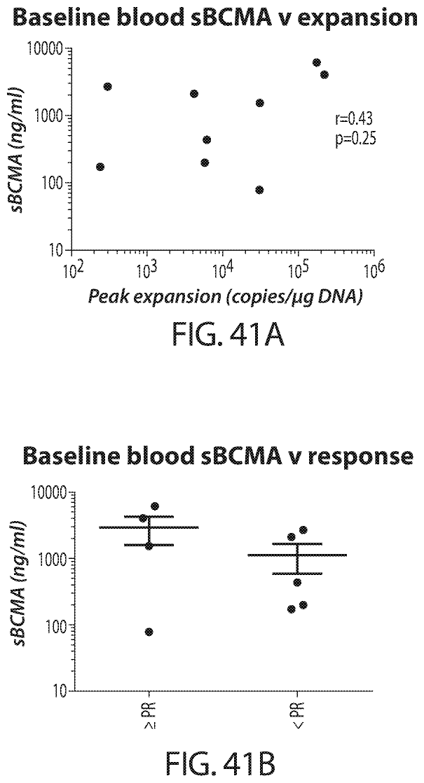

BCMA is shed from the surface of plasma cells by gamma-secretase-mediated cleavage (Laurent et al., Nat Commun 6, 7333 (2015)), leading to a soluble form (sBCMA) that is detectable in circulation. Elevated sBCMA levels are found in myeloma patients and higher concentrations of sBCMA are associated with poorer clinical outcomes (Sanchez et al., Br. J. Haematol. 158, 727-738 (2012)). The impact of CART-BCMA on serum concentration of sBCMA, as well as its ligands BAFF and APRIL, was serially assessed. Compared to a panel of healthy donors (HD, n=6, median sBCMA 41.6 ng/ml, range 25.2-84.4), most of the subjects had an elevated sBCMA concentration in blood at baseline (median 1532 ng/ml, range 78.2-6101.3), along with concomitant suppression of APRIL (median 0.04 ng/ml, range 0.01-0.96, compared to HD, median 5.69 ng/ml, range 3.07-6.24). BAFF concentrations (median 0.84 ng/ml, range 0.51-4.98) were not significantly different from HD (median 0.93 ng/ml, range 0.58-1.24). Baseline sBCMA concentration did not correlate with response (FIG. 41), but decline in sBCMA concentration after CART-BCMA was most pronounced in subjects with the deepest responses (01, 03, 15). sBCMA also started to rise again at time of progression for subjects 03, 07, 15 (FIG. 34), suggesting that sBCMA concentration in blood may be a useful biomarker for assessing myeloma disease burden.

All subjects had depressed frequencies of blood CD19+ B cells at baseline (median 1.9% of CD45+CD14− gate, range 0.1%-4.5%), likely due to immune suppression from progressive myeloma and extensive prior therapy. However, in contrast to patients treated with CD19-directed CAR T cells, which cause prolonged B cell aplasia (Maude et al., N. Engl. J. Med. 371, 1507-1517 (2014)), 6 of 9 subjects treated with CART-BCMA cells had recovery of B cells, typically 2-3 months post-infusions. B cell recovery was most pronounced in subjects with the deepest responses (01, 03, 15), and was generally, though not always, associated with an increase in the concentration of serum BAFF and/or APRIL (FIG. 34), ligands known to promote normal B cell development, proliferation, and survival (Rickert et al., Immunol. Rev. 244, 115-133 (2011)). Importantly, B cell frequency remains normal in subject 01 despite prolonged persistence of circulating CART-BCMA cells, consistent with the previously-reported absence of BCMA expression on the majority of circulating B cells (Seckinger et al., Cancer Cell 31, 396-410 (2017); O'Connor et al., J. Exp. Med. 199, 91-98 (2004)).

### BCMA Expression on Myeloma Cells

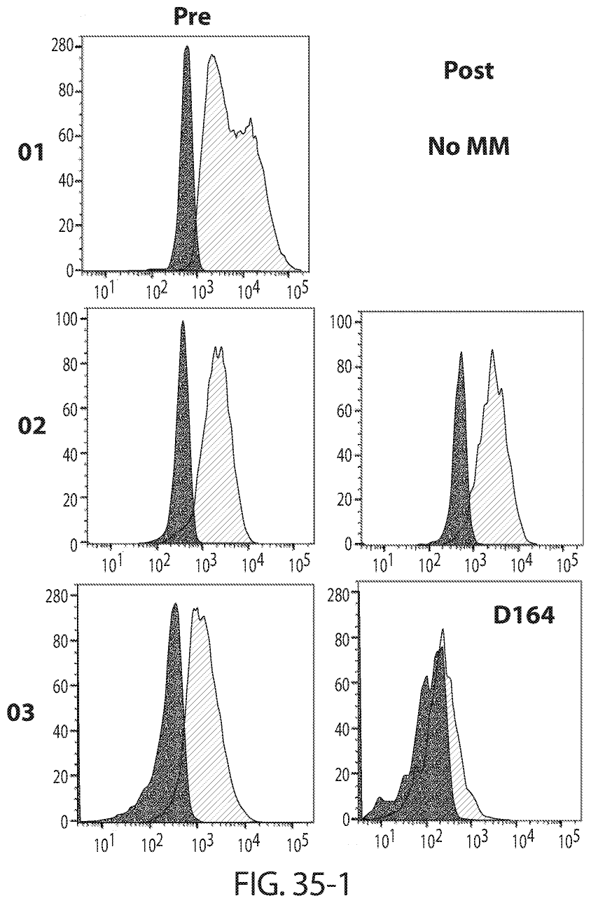

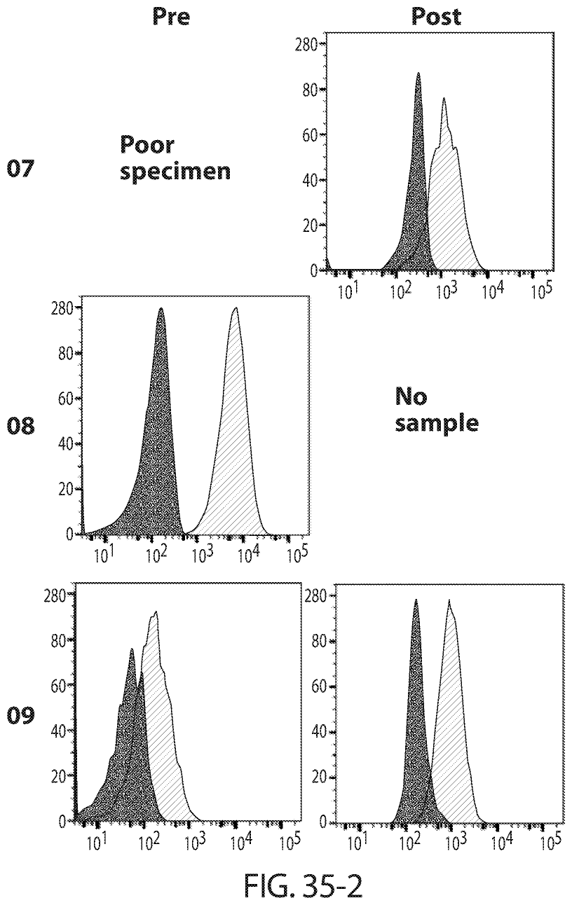

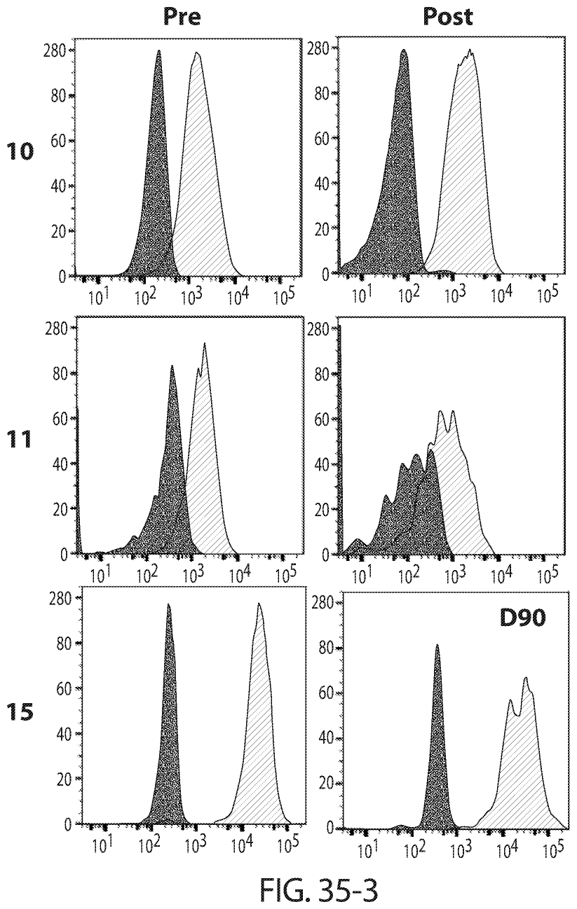

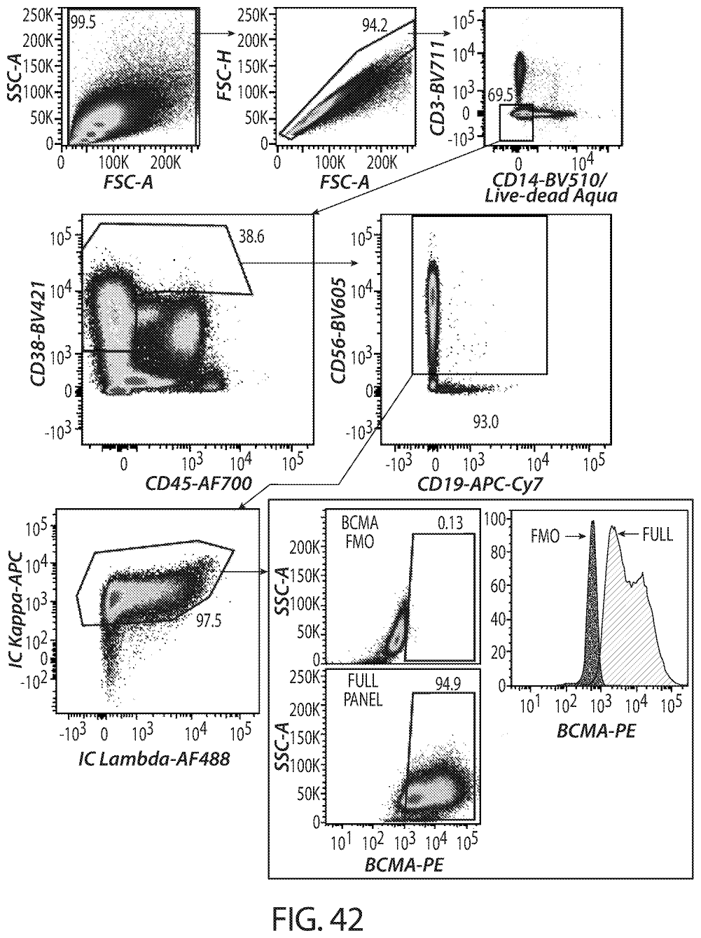

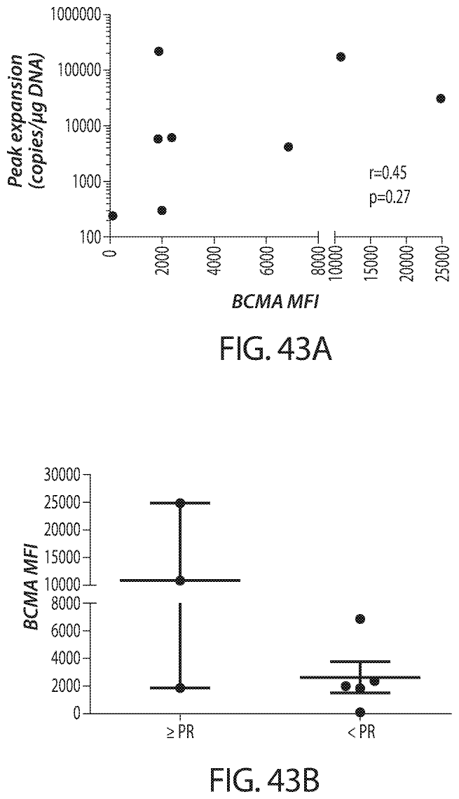

Eight subjects were evaluable for BCMA expression by flow cytometry on myeloma cells prior to treatment, and all had detectable BCMA expression (FIG. 35) (see FIG. 42 for representative gating). Baseline intensity of BCMA varied from subject to subject, and did not appear to correlate with degree of CART-BCMA expansion or response (FIG. 43) in this small cohort. Post-treatment, 7 subjects had myeloma cells evaluable for BCMA expression. One subject (03) had significantly decreased BCMA staining intensity, relative to the fluorescence minus one (FMO) control, at time of progression (day 164) compared to pre-treatment, suggesting either downregulation of surface expression, immune selection of BCMA-dim/negative variants, and/or increased shedding from the cell surface.

### Predictors of CART-BCMA Expansion

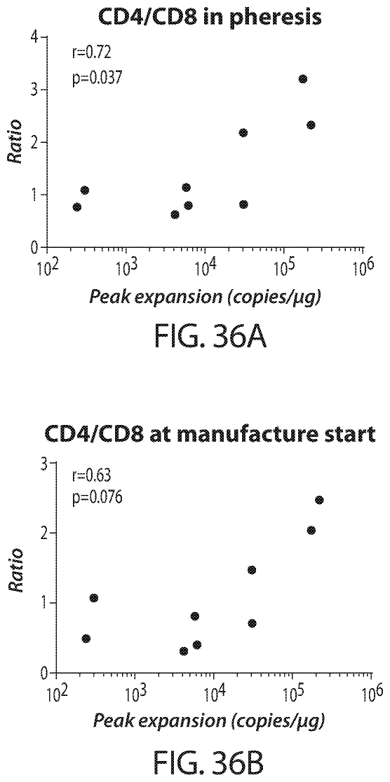

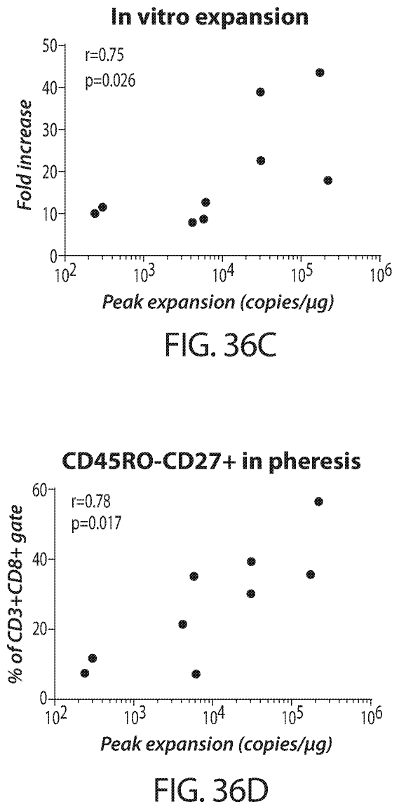

As described above, and consistent with prior CAR T cell studies (Turtle et al., Sci. Transl. Med. 8, 355ra116 (2016); Porter et al., Sci. Transl. Med. 7, 303ra139 (2015); Ali et al., Blood 128, 1688-1700 (2016)), responding subjects had greater expansion and persistence of CART-BCMA cells, and more profound cytokine release, than non-responders. In order to explore pre-treatment characteristics potentially associated with strong expansion, features of the CART-BCMA product before, during, and at end of manufacturing were analyzed. It was found that a higher CD4/CD8 ratio in the leukapheresis product, as well as in the seed culture at the start of manufacturing (i.e. following the elutriation step to remove monocytes), was associated with greater CART-BCMA expansion in subjects (FIGS. 36A and 36B), while total CD3 T cell number in the leukapheresis product or seed culture, or CD4/CD8 ratio in final product at end of manufacturing was not (data not shown). Fold expansion of seeded cells during manufacturing also correlated with in vivo CART-BCMA expansion (FIG. 36C), suggesting that in vitro proliferative capacity may predict for in vivo activity. Finally, previous analyses in CLL patients receiving CD19-directed CAR T cells had demonstrated that better CAR T cell expansion and clinical responses were associated with a higher percentage of CD8+ T cells within the leukapheresis specimen expressing a CD27+CD45RO− phenotype (24). CD8+ T cells within the leukapheresis products of 9 subjects treated with CART-BCMA cells alone were examined and a similar correlation was found between the frequency of CD27+CD45RO− cells and CART-BCMA expansion in vivo (FIG. 36D).

### DISCUSSION

CAR T cell therapy is emerging as a promising therapeutic option for B-cell malignancies, with the potential for durable disease control following a single treatment, differentiating it from other therapies that require repeated and/or continuous administration. In this report, the potential of CAR T cell therapy in advanced and refractory myeloma is demonstrated, with 4/9 subjects achieving a partial response or better, including an ongoing stringent complete remission 21 months post-infusion, as well as 2 additional subjects with minor responses. This is notable given the highly adverse biological features of the enrolled subjects' myeloma, including high tumor burden, rapidly progressing disease, and high-risk genetics. CAR T cell products were successfully manufactured from all subjects, despite baseline T cell lymphocytopenia, and engraftment was seen in all subjects as well, though peak levels and persistence of CAR T cells varied significantly amongst subjects.

Myeloma has long been associated with quantitative and functional deficits in T cells, particularly in more advanced, refractory disease, with inverted CD4/CD8 T cell ratios, impaired ex vivo anti-tumor activity, and acquisition of an exhausted or senescent phenotype (Kay, et al., Blood 98, 23-28 (2001); Dhodapkar, et al., J. Exp. Med. 198, 1753-1757 (2003); Suen, et al., Leukemia 30, 1716-1724 (2016)). In this study responses correlated with degree of in vivo expansion, which in turn was associated with higher pre-manufacturing CD4/CD8 T cell ratio, pre-manufacturing frequency of CD45RO−CD27+CD8+ T cells, and magnitude of in vitro proliferation during manufacturing. This suggests that more effective CART-BCMA products may be derived from subjects with a less differentiated, more “naïve-like” T cell compartment, as previously observed in a CLL trial using CD19-directed CAR T cells (Fraietta, et al., Blood 128, 57-57 (2016)). These findings suggest that pre-treatment phenotypic and/or functional T cell characteristics may eventually help predict subjects likely to respond or not to CART-BCMA therapy. They also suggest that treatment of patients earlier in the course of their disease, when T cells may be intrinsically “fitter,” may be more effective.

The observed CAR T cell expansion and clinical activity is also notable due to the lack of any chemotherapy given as pre-infusion conditioning in this cohort. Chemotherapeutics such as cyclophosphamide have been demonstrated to enhance T cell-mediated anti-tumor immunity via multiple potential mechanisms, including reduction of cellular “sinks” leading to increased availability of IL-7 and IL-15; depletion of suppressor cell populations such as regulatory T cells; induction of mucosal damage with release of Toll-like receptor agonists which enhance maturation of antigen-presenting cells; and alteration of gut microbiota (Gattinoni, et al., Nat. Rev. Immunol. 6, 383-393 (2006); Viaud, et al., Science 342, 971-976 (2013)). Thus, adoptive transfer of tumor-specific T cells, including CAR T cells, in humans has most commonly followed some form of lymphodepleting conditioning (Porter, et al., Sci. Transl. Med. 7, 303ra139 (2015); Rapoport, et al., Nat. Med. 21, 914-921 (2015); Noonan, et al., Sci. Transl. Med. 7, 288ra278 (2015); Morgan, et al., Science 314, 126-129 (2006)), with some studies showing that incremental increases in the intensity of the conditioning leads to even better engraftment and clinical outcomes (Turtle, et al., Sci. Transl. Med. 8, 355ra116 (2016); Dudley, et al., J. Clin. Oncol. 26, 5233-5239 (2008)). Consistent with this, early studies of CAR T cell therapy given without lymphodepleting conditioning saw only low-level expansion and limited persistence of transferred T cells, though admittedly these studies used first-generation CAR constructs that lacked co-stimulatory domains and contained immunogenic sequences that also contributed to poor engraftment (Pule, et al., Mol. Ther. 15, 825-833 (2007); Park, et al., Mol. Ther. 15, 825-833 (2007); Till, et al., Blood 112, 2261-2271 (2008)).

This study clearly demonstrates, however, that at least in some patients (e.g. subject 01), chemotherapy conditioning is not required for robust and sustained CAR T cell engraftment and clinical efficacy. The conditions that may have contributed to this success include the inclusion of the 4-1BB co-stimulatory domain in the CAR construct, high tumor burden with widespread availability of antigen, and the significant baseline lymphopenia in the patient population. Nonetheless, given the known beneficial effects of chemotherapy conditioning as described above, it remains likely that lymphodepletion will increase the proportion of subjects who have successful CART-BCMA expansion and persistence, and perhaps clinical responses. This question will be addressed in Cohorts 2 and 3 which receive cyclophosphamide prior to CART-BCMA cells.

Together with the previous BCMA-specific CAR T cell trial reported by the NCI (Ali, et al., Blood 128, 1688-1700 (2016)), this study validates BCMA as a highly attractive target in myeloma. This has been reinforced further by the promising pre-clinical and early clinical activity noted with bispecific antibodies and antibody-drug conjugates targeting BCMA (Tai, et al., Blood 123, 3128-3138 (2014); Hipp, et al., Leukemia 31, 1743-1751 (2017); Cohen, et al., Blood 128, 1148-1148 (2016)). In addition, preliminary reports of two other studies of BCMA-specific CAR T cells, given in conjunction with lymphodepleting chemotherapy and in less-heavily pretreated patient populations, have shown even higher response rates, several of which were durable for >1 year at time of reporting (Fan, et al., J. Clin. Oncol. 35, abstr LBA3001 (2017); Berdeja, et al., J. Clin. Oncol. 35, abstr 3010 (2017)). Importantly, none of these studies reported any unexpected off-target or off-tumor toxicity, confirming the limited normal tissue expression of BCMA, and differentiating it from other potential CAR T cell targets in myeloma (e.g. CD138, CD38, CS1/SLAMF7) with more widespread expression (Jiang, et al., Mol. Oncol. 8, 297-310 (2014); Drent, et al., Haematologica 101, 616-625 (2016); Chu, et al., Clin. Cancer Res. 20, 3989-4000 (2014)).

An important unanswered question for BCMA-targeted CAR T cells is whether there is a threshold of BCMA expression on MM cells required for optimal recognition and killing. This study did not require any specific level of BCMA as an eligibility requirement, in contrast to the 3 other reported BCMA-specific CAR T cell studies, which required at least 50% of myeloma cells to express BCMA by immunohistochemistry (IHC) or flow cytometry (Ali, et al., Blood 128, 1688-1700 (2016); Fan, et al., J. Clin. Oncol. 35, abstr LBA3001 (2017); Berdeja, et al., J. Clin. Oncol. 35, abstr 3010 (2017)). In the NCI trial, only 52/85 (62%) of pre-screened bone marrow biopsies stained for BCMA by IHC met this threshold, meaning more than a third of potentially eligible MM patients would have been excluded (Ali, et al., Blood 128, 1688-1700 (2016)). While it remains possible that this approach may enrich for patients more likely to respond, it may also exclude those who could benefit, and at least in this initial cohort in this study, responses did not correlate with baseline BCMA expression by flow cytometry (FIG. 43). Larger datasets are needed to more fully address this question. Another question relates to potential downregulation of BCMA or selection of BCMA-dim/negative variants post-CAR T cell therapy, as observed in 1 patient each to date in both this study and the NCI study (Ali, et al., Blood 128, 1688-1700 (2016)). Larger studies and longer follow-up will help determine the true frequency of this phenomenon, but it suggests that it may be a means of MM cell escape.

The primary toxicities of CAR T cells remain cytokine release syndrome (CRS) and neurotoxicity. The frequency and severity of CRS in this cohort was similar to that reported in CD19-targeted CAR T cell trials (Maude, et al., N. Engl. J. Med. 371, 1507-1517 (2014); Porter, et al., Sci. Transl. Med. 7, 303ra139 (2015)), and fortunately was abrogated with IL-6 receptor blockade therapy. Given the apparent lack of impact of tocilizumab on expansion and persistence of infused CAR T cells, studies are now underway (e.g. NCT02906371) exploring its use earlier post-infusion, even if CRS is only low-grade, which may limit the development of severe or life-threatening toxicity. Neurotoxicity has been reported in up to 50% of subjects in some CAR T cell trials (Turtle, et al., Sci. Transl. Med. 8, 355ra116 (2016); Turtle, et al., J. Clin. Invest. 126, 2123-2138 (2016); Kochenderfer, et al., J. Clin. Oncol. 33, 540-549 (2015)), and remains more of a challenge. It can occur concurrent with or subsequent to CRS, and often does not improve (or may worsen) after tocilizumab. The presence of CAR T cells within the central nervous system (CNS) (as assessed by analysis of cerebrospinal fluid) by itself does not necessarily predict for neurotoxicity, but neurotoxicity has been associated with early onset of CRS and rapid elevation of inflammatory cytokines (e.g. IL6, IFN-gamma) both within the serum and CNS, perhaps leading to increased CNS vascular permeability (Gust, et al., Cancer Discov., (2017)). Mild cases are treated supportively, with steroids usually administered for more severe cases. Fortunately, the majority of cases have been reversible and self-limiting, though fatal cases with cerebral edema have been reported (Abbasi, et al., JAMA 317, 2271 (2017); Gust, et al., Cancer Discov., (2017)). The experience in this study demonstrating rapid reversal of a PRES-like syndrome in subject 03 using cyclophosphamide suggests this may be a reasonable option to try in steroid-refractory cases, especially if symptoms are associated with extensive concomitant CAR T cell expansion.

In summary, autologous T cells expressing a fully human BCMA-specific CAR could expand and induce objective responses, even without lymphodepleting chemotherapy, in refractory MM patients. Subsequent cohorts exploring different dose levels in conjunction with cyclophosphamide conditioning will help further optimize the safety and efficacy of this approach.

### Materials and Methods

### Subjects

Subjects had multiple myeloma that had relapsed or was refractory (defined as progressing on or within 60 days of most recent therapy) after at least 3 prior lines of therapy, or 2 prior lines if dual-refractory to a proteasome inhibitor and IMiD. Other key eligibility criteria included measurable disease; an ECOG performance status of 0-2; serum creatinine ≤2.5 mg/dL or estimated creatinine clearance ≥30 ml/min; absolute neutrophil count ≥1000/μl and platelet count ≥50,000/μl (≥30,000/μl if bone marrow plasma cells are ≥50% of cellularity); SGOT ≤3× the upper limit of normal and total bilirubin ≤2.0 mg/dl (except for patients in whom hyperbilirubinemia is attributed to Gilbert's syndrome); left ventricular ejection fraction ≥45%; lack of active auto-immune disease; and lack of central nervous system involvement with myeloma. All subjects had a baseline MRI of the brain and serial evaluations by a designated study neurologist. Informed consent was obtained from each subject and the study was conducted with approval of University of Pennsylvania IRB in concordance with the Declaration of Helsinki.

### Study Design

This clinical trial was a phase 1 open-label study where the primary objective was safety. Toxicity grade was determined according to National Cancer Institute's Common Terminology Criteria for Adverse Events version 4.0, with the exception of cytokine release syndrome, which was graded as per the University of Pennsylvania CRS Grading System (Table 38), as described (Porter, et al., Sci. Transl. Med. 7, 303ra139 (2015)). The study was approved by the Recombinant DNA Advisory Committee, FDA, Abramson Cancer Center Clinical Trials Scientific Review Committee, and the Penn Institutional Biosafety Committee and Institutional Review Board. The trial was registered in clinicaltrials.gov under NCT02546167, and was conducted as described in “Protocol design and enrollment” in the Results section and in FIG. 31. Myeloma responses were assessed by updated International Myeloma Working Group criteria (Kumar, et al., Lancet Oncol. 17, e328-346 (2016), hereby incorporated herein by reference in its entirety). Data cutoff for this analysis was 9/11/17.

### CART-BCMA Manufacturing and Infusions

Peripheral blood T cells were stimulated and transduced with a lentiviral vector encoding the CAR: human anti-BCMA single chain variable fragment fused to the hinge and transmembrane domain of CD8 and the human 4-1BB and CD3z intracellular signaling domains. CART-BCMA cells were manufactured at the Clinical Cell and Vaccine Production Facility at the University of Pennsylvania, which is FACT accredited (http://www.factwebsite.org), as previously described (Porter, et al., Sci. Transl. Med. 7, 303ra139 (2015); Kalos, et al., Sci. Transl. Med. 3, 95ra73 (2011)). The frequency of CD3, CD4, and CD8 cells was determined by flow cytometry within the leukapheresis product, in the seed culture at start of manufacturing (following elutriation to reduce monocytes) (Powell, et al., Cytotherapy 11, 923-935 (2009)), and at end of manufacturing. Fold expansion and population doublings of seeded cells was measured by cell counting on a Coulter Multisizer™. CART-BCMA cells were formulated and cryopreserved until time of infusion, then administered following performance of quality control testing and quality assurance review by intravenous infusion over 3 days, with 10%, 30%, and 60% of the dose given on each day.

### Measurement of CART-BCMA Expansion

Research sample processing, freezing, and laboratory analyses were performed in the Translational and Correlative Studies Laboratory at the University of Pennsylvania, using established standard operating procedures (SOPs) for sample receipt, processing, freezing, and analysis. CART-BCMA cells were quantified from peripheral blood or bone marrow samples obtained at protocol-specified time points. Samples were collected in lavender top (K2EDTA) or red top (no additive) Vacutainer tubes (Becton Dickinson). Lavender top tubes were delivered to the laboratory within 2 hours of the sample draw. Samples were processed within 16 hours of drawing according to the established SOP. PBMCs were purified, processed, and stored in the vapor phase of liquid nitrogen. Red top tubes were processed within 2 hours of the draw including coagulation time, and serum was isolated by centrifugation, aliquoted, and stored at −80° C.

Cells were evaluated by flow cytometry directly after Ficoll-Paque processing. Immunophenotyping of PBMC was performed using about 2×10⁵ to 5×10⁵ total cells per condition depending on cell yield in samples. FMO (fluorescence minus one) secondary only controls were used for CART-BCMA and for BCMA evaluation. Reagents and protocols used for flow cytometry are described in the Supplementary Methods.

Genomic DNA was isolated directly from whole blood or marrow aspirate, and qPCR analysis was performed using ABI TaqMan technology and a validated assay to detect the integrated CAR transgene sequence as described (Kalos, et al., Sci. Transl. Med. 3, 95ra73 (2011)) using triplicates of 200 ng of genomic DNA per time point for peripheral blood and marrow samples. To determine copy number per unit DNA, an eight-point standard curve was generated consisting of 5 to 10⁶ copies of lentivirus plasmid spiked into 100 ng of non-transduced control genomic DNA. The number of copies of plasmid present in the standard curve was verified using digital qPCR with the same primer/probe set and performed on a QuantStudio 3D digital PCR instrument (Life Technologies). Each datapoint (sample and standard curve) was evaluated in triplicate with a positive Ct value in three of three replicates with percent coefficient of variation of less than 0.95% for all quantifiable values. To control for the quality of interrogated DNA, a parallel amplification reaction was performed using 20 ng of genomic DNA and a primer/probe combination specific for a non-transcribed genomic sequence upstream of the CDKN1A (p21) gene as described (Kalos, et al., Sci. Transl. Med. 3, 95ra73 (2011)). These amplification reactions generated a correction factor to adjust for calculated versus actual DNA input. Copies of transgene per microgram of DNA were calculated according to the formula: copies per microgram of genomic DNA=(copies calculated from CART-BCMA standard curve) x correction factor/(amount DNA evaluated in nanograms)×1000 ng.

### Measurement of Serum Cytokines

Human cytokine magnetic 30-plex panel (LHC6003M) was from Life Technologies.

Serum samples collected 1 day prior to CART-BCMA infusion or from baseline and at scheduled time-points out until 28 days post-infusion were cryopreserved at −80° C. Batched samples were thawed and analyzed according to the manufacturers' protocols. Assay plates were measured using a FlexMAP 3D instrument, and data acquisition and analysis were done using xPONENT software. Data quality was examined based on the following criteria. The standard curve for each analyte has a 5P R² value >0.95 with or without minor fitting using xPONENT software. To pass quality control, the results for an in-house control serum needed to be within the 95% of CI (confidence interval) derived from historical in-house control data for >25 of the tested analytes. No further tests were done on samples with results out of range low (<OOR). Samples with results that were out of range high (>OOR) or greater than two times the standard curve maximum value (SC max) were re-tested at higher dilutions. Results that passed the above quality controls or retests were reported.

### Measurement of Soluble BCMA, BAFF, and APRIL

Antibody sets for human BCMA (DY193), APRIL (DY884B) and BAFF (DT124-05) were from R&D Systems. ELISA bead-strip and 4-column reservoir (SOW-A16735) were from Assay Depot. ELISA substrate ADHP (Ser. No. 10/010,469) was from Cayman Chemical. Assay plates (OX1263) were from E&K Scientific. All ELISA reagents were prepared according to the protocols for DuoSet ELISA except for Color Reagent B, which was supplemented with ADHP at 100 uM. Due to limited volumes of sera and lack of availability of a Luminex assay for BCMA, APRIL and BAFF, the three analytes were measured using ELISA bead-strips. Instead of coating the capture antibody (cAB) to the wells of an ELISA plate, it was coated on the surfaces of macrospheres, which enabled the measurement of all three analytes using 100 ul of serum. Assays were set up using bead-strips in assay plates based on an assay map following the protocol for antibody sets. At the end of the assay, one substrate plate per 12 bead-strips was prepared by adding 100 ul/well of substrate solution (1:1 of color reagent A and ADHP). Each bead-strip was placed in one column of the substrate plate according to the assay map. Color development was for 10 to 30 minutes. Plates were read on a FLUO STAR OMEGA instrument. Data quality control was performed as described for Luminex data.

### Assessment of Bone Marrow Myeloma Cells, Including BCMA Expression

Flow cytometry assessment of bone marrow aspirate material was performed directly on aspirate following a brief ammonium chloride red blood cell lysis step. The procedure was adapted from the EuroFlow protocol as described in (Flores-Montero, et al., Leukemia 31, 2094-2103 (2017)). Briefly, up to 2 mls of bone marrow aspirate was diluted with 48m1s of Pharm Lyse solution (BD Biosciences Cat #555899) and incubated for 15 minutes at room temperature on a shaking device. The cells were then collected by centrifugation for 10 minutes at 800 g, washed twice with flow cytometry buffer (PBS with 1% fetal bovine serum), stained with L/D Aqua viability dye (Thermo Fisher Cat # L34957). Surface staining was done with a mixture of antibodies to CD45, CD19, CD138, CD38, CD14, CD56, CD20, CD3, CD269 (BCMA), CD274 (PD-L1). FMO (fluorescence minus one) secondary only controls were used for BCMA evaluation. Aliquots of normal donor PBMC cells were stained in parallel as controls. The cells were then washed before permeabilization/fixation using Cytofix/Cytoperm reagent (BD Biosciences) for 20 minutes at room temperature, washed, and stained with a mixture of antibodies to kappa and lambda immunoglobulin light chains. The samples were then washed before resuspension in PBS and acquisition on a 17-color LSR Fortessa Special Order Research Product flow cytometer (BD) equipped with a violet, blue, green, and red laser. List mode files were analyzed using either FlowJo (Treestar) or FCS Express.

### Statistics

Statistical analysis of the study is primarily descriptive due to the pilot nature of the trial and small sample size of patients. Kaplan-Meier method was used to estimate duration of response, progression-free and overall survival and the associated median survival times. The Mann-Whitney test was used to evaluate significance of the association between response and peak CART-BCMA expansion, area under the curve for expansion over the first 28 days (AUC-28), baseline soluble BCMA levels, and baseline BCMA expression on MM cells. Spearman correlations were used to measure the correlations between two continuous variables. Significance of the Spearman correlation against the null hypothesis of no correlation was computed based on permutation. Analysis was performed using Graphpad Prism version 6.0.

TABLE 30
Subject characteristics

| Characteristic (n = 9) | Median (range) or % | |
|---|---|---|
| Age | 57 | (44-70) |
| Gender | 67% male; 33% female | |
| Median time from diagnosis (years) | 4.9 | (1.8-9.6) |
| High-risk cytogenetics* | 100% | |
| Del 17p or TP53 mutation | 67% | |
| Prior lines of therapy | 9 | (4-11) |
| Len/Bort/Pom/Carf/Dara (% exposed) | 100%/100%/100%/100%/44% | |
| Len/Bort/Pom/Carf/Dara (% refractory) | 78%/89%/89%/89%/44% | |
| % Dual-/% Quad-/% Penta-refractory | 100%/56%/33% | |
| Autologous SCT | 78% | |
| Extramedullary disease | 22% | |
| % Bone marrow plasma cells | 80 | (15-95) |
| Day 0 absolute lymphocyte count | 500 | (200-1420) |
| (cells/μL) | | |
| Day 0 absolute CD3 count (cells/μL) | 258 | (117-1354) |
| Day 0 LDH (xx/xx) | 175 | (109-385) |
| Day 0 serum creatinine (mg/dL) | 1.58 | (0.83-2.87) |
| Day 0 hemoglobin (g/dL) | 8.7 | (6.4-11.3) |
| Day 0 platelets (×10³/ uL) | 100 | (13-221) |

*Includes complex karyotype, gain 1q, deletion 17p, and/or t(4; 14).
Bort = bortezomib; Carf = carfilzomib; Dara = daratumumab; Del = deletion; Dual-refractory = refractory to both a proteasome inhibitor (PI) and immunomodulatory agent (IMiD); LDH = lactate dehyrdrogenase; Len = lenalidomide; Penta-refractory = refractory to 2 Pls, 2 IMiDs, and dara; Pom = pomalidomide; Quad-refractory = refractory to 2 Pls and 2 IMiDs; SCT = stem cell transplant.

TABLE 31
Grade 3 or higher adverse events

| | Grade 3 | Grade 4 | Grade 5 | All |
|---|---|---|---|---|
| Event | n (%) | n (%) | n (%) | n (%) |
| Hypophosphatemia | 4 (44%) | 0 | 0 | 4 (44%) |
| Cytokine release syndrome | 2 (22%) | 1 (11%) | 0 | 3 (33%) |
| Thrombocytopenia | 2 (33%) | 1 (11%) | 0 | 3 (33%) |
| Neurotoxicity* | 0 | 2 (22%) | 0 | 2 (22%) |
| Anemia | 2 (22%) | 0 | 0 | 2 (22%) |
| Lung infection | 0 | 2 (22%) | 0 | 2 (22%) |
| Neutropenia | 0 | 2 (22%) | 0 | 2 (22%) |
| Hypocalcemia | 1 (11%) | 1 (11%) | 0 | 2 (22%) |
| Hypokalemia | 2 (22%) | 0 | 0 | 2 (22%) |
| Death NOS** | 0 | 0 | 1 (11%) | 1 (11%) |
| Fatigue | 1 (11%) | 0 | 0 | 1 (11%) |
| Infection-other (candidemia) | 0 | 1 (11%) | 0 | 1 (11%) |
| Urinary tract infection | 1 (11%) | 0 | 0 | 1 (11%) |
| Alkaline phosphatase elevated | 1 (11%) | 0 | 0 | 1 (11%) |
| AST elevated | 1 (11%) | 0 | 0 | 1 (11%) |
| Fibrinogen decreased | 1 (11%) | 0 | 0 | 1 (11%) |
| Lymphopenia | 0 | 1 (11%) | 0 | 1 (11%) |
| Leukopenia | 0 | 1 (11%) | 0 | 1 (11%) |
| Hyponatremia | 1 (11%) | 0 | 0 | 1 (11%) |
| Pleural effusion | 1 (11%) | 0 | 0 | 1 (11%) |
| Hypertension | 1 (11%) | 0 | 0 | 1 (11%) |

n = number of subjects who had the event. Highest grade toxicity experienced by subject is reported in table.
*Neurotoxicity includes 1 subject with grade 3 seizures, grade 4 delirium, and grade 4 reversible posterior leukoencephalopathy syndrome (RPLS), also known as posterior reversible encephalopathy syndrome (PRES); and 1 subject with grade 3 delirium and grade 4 encephalopathy.
**Death NOS (not otherwise specified) in subject 08 with candidemia and rapidly progressive myeloma; family chose to pursue comfort measures only.

### Supplementary Methods

### Reagents and Protocols for Flow Cytometry

Antibodies for T cell detection panels were anti-CD45 V450 (clone HI30), anti-CD14 V500 (clone M5E2), anti-CD56 Ax488 (clone B159), anti-CD4 PerCP-Cy5.5 (clone RPA-T4), anti-CD8 APC-H7 (clone SK1) (all from BD Bioscience). Also, anti-CD3 BV605 (clone OKT3), anti-HLA-DR BV711 (clone L243), anti-CD19 PE-Cy7 (clone H1B19) were used from Biolegend. CART-BCMA expression was assessed by using a bis-biotinylated BCMA-Fc recombinant protein and the secondary staining reagent Streptavidin-PE from BD Bioscience (cat #554061). Cells were resuspended in 100 μL PBS containing 1% fetal bovine serum, 0.02% sodium azide and bis-biotinylated BCMA-Fc and incubated for 30 min on ice, washed, resuspended in 100 μL PBS containing 1% fetal bovine serum, 0.02% sodium azide, surface antibodies and SA-PE, and incubated for 30 minutes on ice, washed, resuspended in 250 ul PBS containing 1% fetal bovine serum and 0.02% sodium azide and acquired using a Fortessa flow cytometer equipped with a violet (405 nm), blue (488 nm), a green (532 nm), and a red (628 nm) laser. Data were analyzed using FlowJo software (Version 10, Treestar). Compensation values were established using eBiosciene UltraComp eBeads (eBioscience cat #01-222-42) and DIVA software.

TABLE 32
Individual subject characteristics

| | | | | Yrs | Iso- | Karyo- | | | # | Last tx before |
|---|---|---|---|---|---|---|---|---|---|---|
| Sub. | Age | Sex | Race | from Dx | type | type | FISH | Mutation | lines* | pheresis, infusion** |
| 01 | 66 | M | white | 9.6 | IgGK | 46XY | +11, | NRAS, | 11 | Pom/Dex |
| | | | | | | | −16q, | TP53, | | Pom/Dex |
| | | | | | | | −17p | TP53 | | |
| 02 | 68 | M | white | 6.3 | IgAK | 46XY | +1q, | n/a | 9 | Carfilz/Len/Dex |
| | | | | | | | +4p, | | | Carfilz/Cyclo/Dex |
| | | | | | | | −17p | | | |
| 03 | 54 | F | white | 3.1 | IgAE | n/a | +1q, | n/a | 4 | D-PACE |
| | | | | | | | t(4; 14), | | | D-AC |
| | | | | | | | −16q | | | |
| 07 | 44 | M | white | 1.8 | K | n/a | +1q, −4, | NRAS, | 9 | Carfilz/Pano |
| | | | | | | | +11, | TP53 | | Pom/Dex-ACE |
| | | | | | | | −14, −16 | | | |
| 08 | 70 | M | white | 3.3 | IgAL | complex | +1q, | n/a | 6 | Carfilz/Pom/Dex |
| | | | | | | | −1p, −4, | | | Carfilz/Pom/Dex |
| | | | | | | | −17p | | | |
| 09 | 61 | M | white | 6.6 | K | 46XY | t(11; 14), | none | 9 | Daratumumab |
| | | | | | | | −16q, | | | Pulse Dex |
| | | | | | | | −17p | | | |
| 10 | 47 | M | white | 4.9 | IgGL | n/a | +1q, | KRAS, | 7 | Pom/Dex |
| | | | | | | | t(11; 14) | SF3B1 | | Pom/Cyclo/Dex + |
| | | | | | | | | | | plasma exchange |
| 11 | 57 | F | AA | 6.3 | IgGL | 46XX | −1q, | n/a | 10 | VDT-PACE |
| | | | | | | | t(4; 14), | | | VDT-PACE |
| | | | | | | | −17p | | | |
| 15 | 51 | F | Asian | 4.7 | IgAK | 46XX | +1q, | NRAS, | 7 | Pembro/Pom/Dex |
| | | | | | | | t(11; 14) | KRAS, | | Pom/Dex |
| | | | | | | | | BIRC3, | | |
| | | | | | | | | KIT | | |

*A line of therapy was defined as per IMWG criteria. Radiation was not counted as a line.
**Most recent therapy received before T cell collection (“pheresis” - top line) and CART-BCMA infusion (“infusion” - bottom line). All therapy was held for at least 2 weeks prior to pheresis and again for at least 2 weeks prior to infusion. Carfilz = carfilzomib; cyclo = cyclophosphamide; D-AC = dexamethasone + infusional doxorubicin and cyclophosphamide; D-PACE = dexamethasone + infusional cisplatinum, doxorubicin, cyclophosphamide, and etoposide; Dex = dexamethasone; Dx = diagnosis; Len = lenalidomide; Pano = panobinostat; Pembro = pembrolizumab; Pom = pomalidomide; Pom/Dex-ACE = pomalidomide, dexamethasone + infusional doxorubicin, cyclophosphamide, and etoposide; Tx = treatment; VDT-PACE = bortezomib, dexamethasone, thalidomide + infusional cisplatinum, doxorubicin, cyclophosphamide, and etoposide; Yrs = years.

TABLE 33
CART-BCMA manufacturing and product details

| | | | | | | | | | | Total | |
|---|---|---|---|---|---|---|---|---|---|---|---|
| | | | % CD3 + | CD4:CD8 | % CD3 + | CD4:CD8 | | | Trans | #CAR | |
| | % CD3 + | CD4:CD8 | in seed | in seed | at | at | Fold | Pop | Eff | given | Best |
| Sub. | in aph | in aph | culture | culture | harvest | harvest | exp | dblgs | (%) | (×10⁸) | response |
| 01 | 28.4 | 3.20 | 52.6 | 2.04 | 98.4 | 2.40 | 43.5 | 4.54 | 9.6 | 2.0* | sCR |
| 02 | 41.9 | 0.80 | 63 | 0.40 | 69.1 | 1.31 | 12.7 | 3.67 | 18.2 | 5.0 | MR |
| 03 | 25.2 | 2.33 | 58.4 | 2.47 | 97.6 | 1.72 | 17.9 | 4.16 | 25.9 | 2.0* | VGPR |
| 07 | 10.2 | 2.18 | 20.3 | 1.47 | 94.9 | 1.60 | 38.9 | 5.28 | 24.2 | 5.0 | PR |
| 08 | 9.2 | 0.62 | 20.8 | 0.31 | 92.5 | 2.99 | 7.9 | 2.98 | 22.7 | 5.0 | PD |
| 09 | 54.6 | 0.77 | 86.8 | 0.49 | 87.5 | 1.69 | 10.0 | 3.33 | 22.2 | 5.0 | SD |
| 10 | 19.2 | 1.09 | 47.3 | 1.07 | 96.7 | 1.32 | 11.5 | 3.52 | 16.5 | 1.77 | PD |
| 11 | 5.56 | 1.14 | 21.4 | 0.81 | 96.9 | 1.20 | 8.7 | 3.11 | 19.4 | 5.0 | MR |
| 15 | 39.8 | 0.82 | 90.8 | 0.71 | 96 | 1.24 | 22.6 | 4.50 | 33.3 | 2.0* | VGPR |

Frequency of total CD3+, CD3+CD4+, and CD3+CD8+ cells was assessed by flow cytometry in the apheresis product, at the start of manufacturing (“seed culture,” after elutriation), and at the end of manufacturing (“at harvest”). Aph = apheresis product; fold exp = fold expansion; pop dblgs = population doublings; trans eff = transduction efficiency. MR = minimal response; PD = progressive disease; PR = partial response; sCR = stringent complete response; SD = stable disease; VGPR = very good partial response
*Subjects 01, 03, and 15 received only 40% of planned dose due to fevers/early CRS.

TABLE 34
Individual subject adverse events

| Sub. | Grade 1 | Grade 2 | Grade 3 | Grade 4 | Grade 5 |
|---|---|---|---|---|---|
| 01 | Dry eye | DIC | Anemia | Lymphopenia | |
| | Dry mouth | Vertigo | Fatigue | | |
| | Flu-like symptoms | Ileus | CRS | | |
| | Metabolism disorder - | Nausea | Alk phos increased | | |
| | other (iron | Vomiting | AST increased | | |
| | deficiency) | Fever | Hypocalcemia | | |
| | Myalgia | Creatinine increased | Hypokalemia | | |
| | Headache | Thrombocytopenia | Hypophosphatemia | | |
| | Ischemia | Musculoskeletal | | | |
| | cerebrovascular | disorder - other | | | |
| | Nervous system | (generalized muscle | | | |
| | disorder - other | aches) | | | |
| | (confusion) | Urinary retention | | | |
| | Vascular disorder - | Respiratory disorder - | | | |
| | other (edema) | other (pulmonary | | | |
| | | edema and atelectasis) | | | |
| | | Skin/SQ disorder - | | | |
| | | other (skin tear) | | | |
| | | Hypertension | | | |
| 02 | Sinus tachycardia | | Thrombocytopenia | | |
| | Gastrointestinal | | | | |
| | disorders - other | | | | |
| | (loose stool) | | | | |
| | Nausea | | | | |
| | Fatigue | | | | |
| | CRS | | | | |
| | Dizziness | | | | |
| | Headache | | | | |
| | Respiratory disorder - | | | | |
| | other (nasal and | | | | |
| | sinus congestion, | | | | |
| | cough) | | | | |
| 03 | Sinus bradycardia | SVT | CRS | Neutropenia | |
| | Ear disorder - other | Fatigue | UTI | Leukopenia | |
| | (ears feel full, hard | Infection - other (S. | Fibrinogen | Hypocalcemia | |
| | to hear) | mitis/S. oralis | decreased | RPLS | |
| | Eye disorder - other | bacteremia) | Hypophosphatemia | Delirium | |
| | (difficulty reading) | Upper respiratory | Seizure | | |
| | Constipation | infection | Pleural effusion | | |
| | Edema | Lymphopenia | Hypertension | | |
| | Fever | Hypernatremia | | | |
| | Hypokalemia | Musculoskeletal | | | |
| | Hypomagnesemia | disorder - other | | | |
| | Bone pain | (deconditioning, pain | | | |
| | Concentration | base of skull) | | | |
| | impairment | Intracranial | | | |
| | Headache | hemorrhage | | | |
| | Hoarseness | Respiratory disorder - | | | |
| | Skin/SQ disorder - | other (post-nasal drip) | | | |
| | other (ecchymosis) | Hypotension | | | |
| 07 | Skin/SQ disorder - | CRS | Anemia | | |
| | other (rash on face) | Dyspepsia | Hypokalemia | | |
| | | Hypoalbuminemia | Hyponatremia | | |
| | | Hypocalcemia | | | |
| 08 | Sinus tachycardia | | Lung infection | CRS | Death |
| | General disorder - | | Delirium | Thrombocytopenia | NOS |
| | other (rigors) | | | Encephalopathy | |
| | | | | Infection - other | |
| | | | | (fungemia) | |
| 09 | Constipation | Anemia | | | |
| | Chills | Pericardial effusion | | | |
| | Fatigue | CRS | | | |
| | Thrombocytopenia | Neutropenia | | | |
| | Anorexia | Hypocalcemia | | | |
| | Hypokalemia | Headache | | | |
| 10 | Chills | Thromobocytopenia | | | |
| | Fever | | | | |
| 11 | Nausea | CRS | Hypophosphatemia | | |
| | Fatigue | Infection - other | | | |
| | Thrombocytopenia | (Clostridium difficile) | | | |
| | Headache | Hypocalcemia | | | |
| | Respiratory disorder - | | | | |
| | other (cough, | | | | |
| | congestion) | | | | |
| 15 | Hypocalcemia | Fatigue | Thrombocytopenia | Neutropenia | |
| | Headache | CRS | | | |
| | Cough | Infection - other | | | |
| | Dyspnea | (positive test for Shiga | | | |
| | | toxin) | | | |
| | | Hypotension | | | |

All events regardless of attribution are listed. If an event occurred more than once in same patient, highest grade is reported.
Alk phos = alkaline phosphatase; AST = aspartate aminotransferase; CRS = cytokine release syndrome; DIC = disseminated intravascular coagulation; NOS = not otherwise specified; RPLS = reversible posterior leukoencephalopathy syndrome; SQ = subcutaneous; SVT = supraventricular tachycardia; UTI = urinary tract infection

TABLE 35
Characteristics of peripheral blood CART-BCMA + cells at peak expansion

| | Day of | % CAR+ | % CAR+ | % CAR+ | % HLADR+ | |
|---|---|---|---|---|---|---|
| Sub. | peak | of CD3+ | of CD4+ | of CD8+ | of CAR+ | Best response |
| 01 | 10 | 89.5 | 8.6 | 92.3 | 97 | sCR |
| 02 | 9 | 2.8 | 1.4 | 5.0 | 82 | MR |
| 03 | 14 | 76.2 | 13.7 | 85.9 | 97 | VGPR |
| 07 | 10 | 29.2 | 7.3 | 39.3 | 89 | PR |
| 08 | 16 | 9.3 | 0.4 | 16.9 | 95 | PD |
| 09 | 11* | * | * | * | * | SD |
| 10 | 30 | 1.9 | 0.1 | 2.0 | 21 | PD |
| 11 | 8 | 18.2 | 14.6 | 25.2 | 94 | MR |
| 15 | 10 | 28.1 | 6.3 | 51.0 | 96 | VGPR |

CART-BCMA cells were assessed by flow cytometry as in FIG. 38. At day of peak expansion, the frequency of CAR+ cells within CD3+, CD4+, and CD8+ populations are listed. Activation status at peak expansion (as measured by % of CAR+ cells expressing HLA-DR) is also shown. MR = minimal response; PD = progressive disease; PR = partial response; sCR = stringent complete response; SD = stable disease; VGPR = very good partial response
*Peak determined by qPCR; CAR+ cells not detectable by flow.

TABLE 36
CART-BCMA engraftment by qPCR in blood, bone marrow, and other sites

| | Day 28 | Day 28 | 3 month | 3 month | |
|---|---|---|---|---|---|
| Sub. | blood | BM | blood | BM | Other (site, day) |
| 01 | 23447.33 | 10458.66 | 4373.95 | n/a | n/a |
| 02 | 454.57 | 383.62 | 334.90 | n/a | n/a |
| 03 | 220081.08* | 109964.97* | 9517.67 | 9934.97 | CSF, D4: 3285.88 |
| | | | | | CSF, D16: 158038.74 |
| | | | | | Pleural fluid, D8: 23941.97 |
| | | | | | Pleural fluid, D18: 144516.62 |
| | | | | | BM, D164: 105.81 |
| 07 | 1757.60 | 2140.31 | n/a | n/a | n/a |
| 08 | n/a | n/a | n/a | n/a | n/a |
| 09 | 32.15 | 16.75 | n/a | n/a | n/a |
| 10 | 62.64 | 0 | n/a | n/a | n/a |
| 11 | 731.44 | | n/a | n/a | n/a |
| 15 | 724.49 | 1084.16 | 32.58 | 99.48 | n/a |

CART-BCMA levels (copies/μg genomic DNA) were generally comparable in blood and marrow at tested timepoints. CART-BCMA was found at high levels in CSF and pleural fluid of subject 03.
BM = bone marrow; CSF = cerebrospinal fluid.
*Assays performed on day 45.

TABLE 37
Details of BCMA expression on myeloma cells

| | | | | | | Post-Tx | |
|---|---|---|---|---|---|---|---|
| | Pre-tx | Pre-tx | Pre-tx | Post-tx | Post-tx | BCMA | Best |
| Sub. | % BCMA+ | FMO MFI | BCMA MFI | % BCMA+ | FMO MFI | MFI | response |
| 01 | 95 | 568 | 10864 | n/a | n/a | n/a | sCR |
| 02 | 93 | 355 | 2375 | 97 | 483 | 3254 | MR |
| 03 | 84 | 265 | 1873 | 19 | 78 | 268 | VGPR |
| | | | | (d 164) | (d 164) | | |
| 07 | n/a | n/a | n/a | 84 | 273 | 1426 | PR |
| 08 | 99 | 124 | 6859 | n/a | n/a | n/a | PD |
| 09 | 28 | 50 | 103 | 93.7 | 204 | 1116 | SD |
| 10 | 83 | 192 | 1998 | 99 | 54 | 2140 | PD |
| 11 | 81 | 332 | 1844 | 62 | 49 | 831 | MR |
| 15 | 99 | 249 | 24842 | 100 | 362 | 30685 | VGPR |
| | | | | | (d 90) | (d 90) | |

Bone marrow myeloma cells were gated and BCMA expression analyzed. The % of myeloma cells expressing BCMA, as well as the mean fluorescence intensity (MFI) for BCMA and FMO negative control are depicted, n/a = not available. Pre-tx = pre-treatment. Post-tx = post-treatment, at day 28 timepoint unless otherwise specified.

TABLE 38
Penn grading system for Cytokine Release Syndrome (CRS)
University of Pennsylvania CRS Toxicity Grade

| 1 | 2 | 3 | 4 | 5 |
|---|---|---|---|---|
| Mild reaction: | Moderate reaction | More severe reaction: | Life-threatening | Death |
| Treated with | requiring IV fluids | Hospitalization required for | complications | |
| supportive | or parenteral | management of symptoms | such as | |
| care such as | nutrition; some | related to organ dysfunction | hypotension | |
| anti-pyretics, | signs of organ | including grade 4 LFTs or | requiring high | |
| anti-emetics | dysfunction (i.e., | grade 3 creatinine related to | dose pressors or | |
| | grade 2 creatinine | CRS and not attributable to | hypoxia | |
| | or grade 3 liver | any other conditions. This | requiring | |
| | function tests | excludes management of fever | mechanical | |
| | [LFTs] related to | or myalgias. Includes | ventilation. | |
| | CRS and not | hypotension treated with | | |
| | attributable to any | IVFs* or low-dose pressors, | | |
| | other condition). | coagulopathy requiring fresh | | |
| | Hospitalization for | frozen plasma (FFP) or | | |
| | management of | cryoprecipitate, and hypoxia | | |
| | CRS related | requiring supplemental oxygen | | |
| | symptoms including | (nasal cannula oxygen, high | | |
| | fevers with | flow oxygen, Continuous | | |
| | associated | Positive Airway Pressure | | |
| | neutropenia. | [CPAP] or Bilateral Positive | | |
| | | Airway Pressure [BiPAP]). | | |
| | | Patients admitted for | | |
| | | management of suspected | | |
| | | infection due to fevers and/or | | |
| | | neutropenia may have grade 2 | | |
| | | CRS. | | |

*Defined as hypotension requiring multiple fluid boluses for blood pressure support.

### Example 6: BCMA Surface Expression in B Cell Malignancies that are not Multiple Myeloma

### Materials and Methods

The following activated B cell subtype diffuse large B cell lymphoma (DLBCL) cell lines were tested: HBL-1, Oci-Ly3 and TMD-8. Additionally, the following germinal center B cell (GCB) subtype DLBCL lines were tested: SuDHL-4, SuDHL-6, and SuDHL-10. The multiple myeloma (MM) lines U266 and KMS11 served as positive controls and the acute lymphoblastic leukemia line Nalm6 as well as the chronic myelogenous leukemia (CML) line K562 were used as negative controls. Cells were stained with a PE-labeled anti-BCMA antibody (Biolegend, cat #357504) at 1:50 dilution. The antibody binding capacity (ABC) was determined with the Quantum Simply Cellular quantification kit (Bangs Laboratories, cat #815) according to the manufacturer's protocol. Cells were analyzed on a BD Fortessa flow cytometer and data was analyzed using FlowJo.

### Results

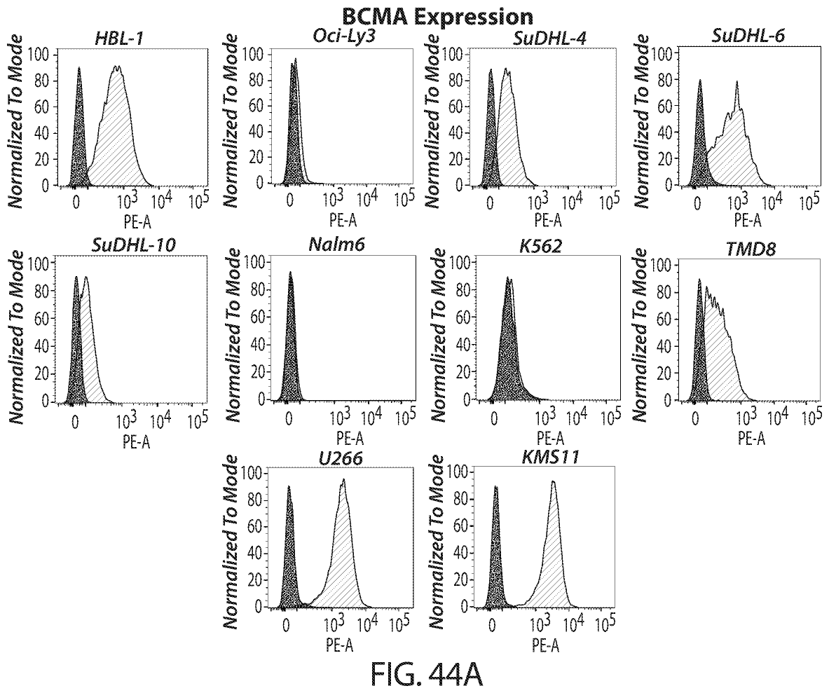

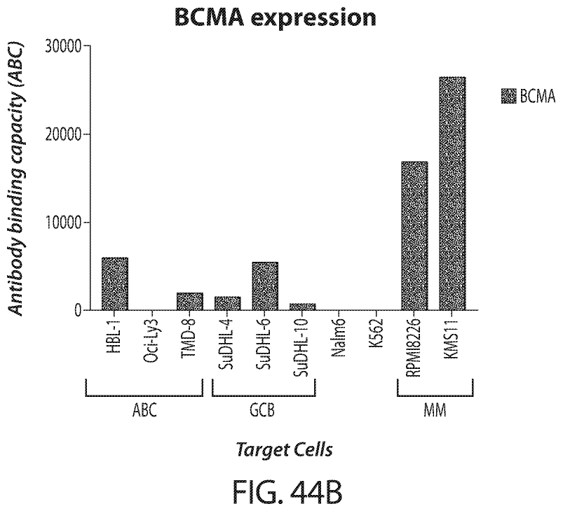

FIG. 44A shows the histograms of BCMA expression and in FIG. 44B, the antibody binding capacity for each cell line is plotted. Both positive control lines showed high BCMA expression as seen in the histograms and by quantification (FIGS. 44A and 44B). All other lines showed significantly lower BCMA expression. However, all DLBCL lines tested, with the exception of Oci-Ly3, were distinctly BCMA positive. HBL-1 and SuDHL-6 showed the highest expression with an antibody binding capacity of >5,000.

### Example 7: Predictors of T Cell Expansion and Clinical Responses Following B-Cell Maturation Antigen-Specific Chimeric Antigen Receptor T Cell Therapy (CART-BCMA) for Relapsed/Refractory Multiple Myeloma (MM)

Background: Relapsed/refractory (rel/ref) MM is associated with progressive immune dysfunction, including reversal of CD4:CD8 T cell ratio and acquisition of terminally-differentiated T cell phenotypes. BCMA-directed CAR T cells have promising activity in MM, but the factors that predict for robust in vivo expansion and responses are not known. In a phase 1 study of CART-BCMA (autologous T cells expressing a human BCMA-specific CAR with CD3ι/4-1BB signaling domains) in refractory MM patients (median 7 priors, 96% high-risk cytogenetics), partial response (PR) or better was observed in 12/25 (47%) (Cohen et al, ASH 2017, #505, herein incorporated by reference in its entirety). Recently, it was demonstrated in CLL patients receiving CD19-directed CAR T cells that certain T cell phenotypes prior to generation of the CAR T product were associated with improved in vivo expansion and clinical outcomes (Fraietta et al, Nat Med 2018, herein incorporated by reference in its entirety). This study thus sought to identify pre-treatment clinical or immunological features associated with CART-BCMA expansion and/or response.

Methods:

Three cohorts were enrolled: 1) 1-5×10⁸ CART cells alone; 2) cyclophosphamide (Cy) 1.5 g/m²+1-5×10⁷ CART cells; and 3) Cy 1.5 g/m²+1-5×10⁸ CART cells. Phenotypic analysis of peripheral blood (PB) and bone marrow (BM) mononuclear cells, frozen leukapheresis aliquots, and phenotype and in vitro kinetics of CART-BCMA growth during manufacturing were performed by flow cytometry. CART-BCMA in vivo expansion was assessed by flow cytometry and qPCR. Responses were assessed by IMWG criteria.

Results:

Responses (≥PR) were seen in 4/9 pts (44%, 1 sCR, 2 VPGR, 1 PR) in cohort 1; ⅕ (20%, 1 PR) in cohort 2; and 7/11 (64%, 1 CR, 3 VGPR, 3 PR) in cohort 3. As of 7/9/2018, 3/25 (12%) remain progression-free at 11, 14, and 32 months post-infusions. As previously described, responses were associated with both peak in vivo CART-BCMA expansion (p=0.002) as well as expansion over first month post-infusion (AUC-28, p=0.002). No baseline clinical or MM-related characteristic was significantly associated with expansion or response, including age, isotype, time from diagnosis, # prior therapies, being quad- or penta-refractory, presence of del 17p or TP53 mutation, serum hemoglobin, BM MM cell percentage, MM cell BCMA intensity, or soluble BCMA concentration. Treatment regimen given before leukapheresis or CART-BCMA infusions also had no predictive value. It was found, however, that higher CD4:CD8 T cell ratios within the leukapheresis product were associated with greater in vivo CART-BCMA expansion (Spearman's r=0.56, p=0.005) and clinical response (PR or better; p=0.014, Mann-Whitney). In addition, and similar to CLL data, it was found that a higher frequency of CD8 T cells within the leukapheresis product with an “early-memory” phenotype of CD45RO−CD27+ was also associated with improved expansion (Spearman's r=0.48, p=0.018) and response (p=0.047). Analysis of manufacturing data confirmed that higher CD4:CD8 ratio at culture start was associated with greater expansion (r=0.41, p=0.044) and, to a lesser degree, responses (p=0.074), whereas absolute T cell numbers or CD4:CD8 ratio in final CART-BCMA product was not (p=NS). In vitro expansion during manufacturing did associate with in vivo expansion (r=0.48, p=0.017), but was not directly predictive of response. At the time of CART-BCMA infusion, the frequency of total T cells, CD8+ T cells, NK cells, B cells, and CD3+CD56+ cells within the PB or BM was not associated with subsequent CART-BCMA expansion or clinical response; higher PB and BM CD4:CD8 ratio pre-infusion correlated with expansion (r=0.58, p=0.004 and r=0.64, p=0.003, respectively), but not with response.

Conclusions:

In this study, it was found that CART-BCMA expansion and responses in heavily-pretreated MM patients were not associated with tumor burden or other clinical characteristics, but did correlate with certain immunological features prior to T cell collection and manufacturing, namely preservation of normal CD4:CD8 ratio and increased frequency of CD8 T cells with a CD45RO−CD27+ phenotype. This suggests that patients with less dysregulated immune systems may generate more effective CAR T cell products in MM, and has implications for optimizing patient selection, timing of T cell collection, and manufacturing techniques to try to overcome these limitations in MM patients.

### Example 8: Clinical Predictors of T Cell Fitness for CAR T Cell Manufacturing and Efficacy in Multiple Myeloma

Introduction:

The optimal clinical setting and cell product characteristics for chimeric antigen receptor (CAR) T cell therapy in multiple myeloma (MM) are uncertain. In CLL patients treated with anti-CD19 CAR T cells (CART19), prevalence of an early memory (early-mem) T cell phenotype (CD27+CD45RO−CD8+) at time of leukapheresis was predictive of clinical response independently of other patient- or disease-specific factors and was associated with enhanced capacity for in vitro T cell expansion and CD19-responsive activation (Fraietta et al. Nat Med 2018, herein incorporated by reference in its entirety). T cell fitness is therefore a major determinant of response to CAR T cell therapy. In this study, leukapheresis samples from MM patients obtained at completion of induction therapy (post-ind) was compared with those obtained in relapsed/refractory (rel/ref) patients for frequency of early-mem T cells, CD4/CD8 ratio, and in vitro T cell expansion.

Methods:

Cryopreserved leukapheresis samples were analyzed for the percentage of early-mem T cells and CD4/CD8 ratio by flow cytometry and in vitro expansion kinetics during anti-CD3/anti-CD28 bead stimulation. Post-ind samples were obtained between 2007 and 2014 from previously reported MM trials in which ex-vivo-expanded autologous T cells were infused post-ASCT to facilitate immune reconstitution (NCT01245673, NCT01426828, NCT00046852); rel/ref samples were from MM patients treated in a phase-one study of CART-BCMA (NCT02546167).

Results:

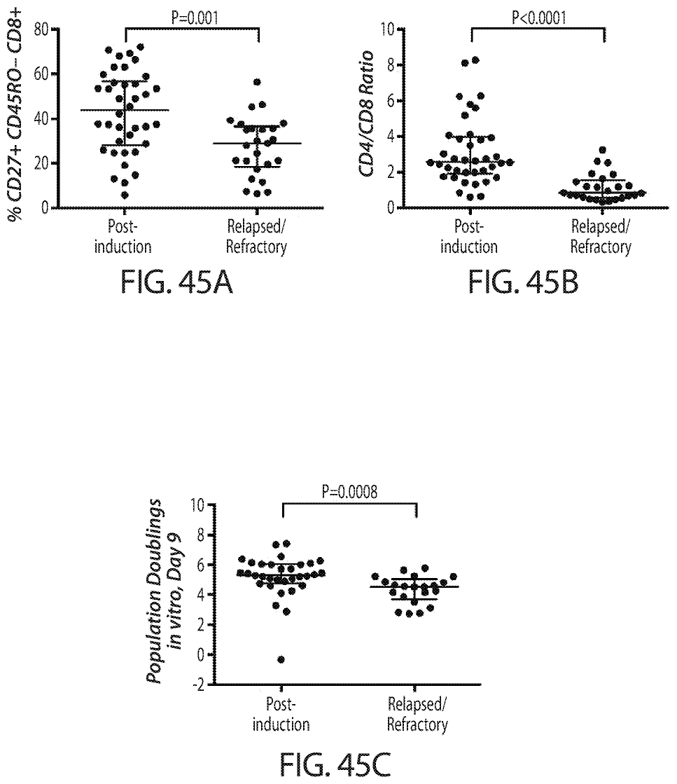

The post-ind cohort includes 38 patients with median age 55y (range 41-68) and prior exposure to lenalidomide (22), bortezomib (21), dexamethasone (38), cyclophosphamide (8), vincristine (2), thalidomide (8), and doxorubicin (4); median time from first systemic therapy to leukapheresis was 152 days (range 53-1886) with a median of 1 prior line of therapy (range 1-4). The rel/ref cohort included 25 patients with median age 58y (range 44-75), median 7 prior lines of therapy (range 3-13), and previously exposed to lenalidomide (25), bortezomib (25), pomalidomide (23), carfilzomib/oprozomib (24), daratumumab (19), cyclophosphamide (25), autologous SCT (23), allogeneic SCT (1), and anti-PD1 (7). Median marrow plasma cell content at leukapheresis was lower in the post-ind cohort (12.5%, range 0-80, n=37) compared to the rel/ref cohort (65%, range 0-95%). Percentage of early-mem T cells was higher in the post-ind vs rel/ref cohort (median 43.9% vs 29.0%, p=0.001, FIG. 45A). Likewise, CD4/CD8 ratio was higher in the post-ind vs rel/ref cohort (median 2.6 vs 0.87, p<0.0001, FIG. 45B). Magnitude of in vitro T cell expansion during manufacturing (measured as population doublings by day 9, or PDL9), which correlated with response to CART19 in CLL, was higher in post-ind vs rel/ref cohort (median PDL9 5.3 vs 4.5, p=0.0008, FIG. 45C). Pooling data from both cohorts, PDL9 correlated with both early-mem T cell percentage (Spearman's rho 0.38, multiplicity adjusted p=0.01) and CD4/CD8 ratio (Spearman's rho 0.42, multiplicity adjusted p=0.005). Within the post-ind cohort, there was no significant association between early-mem T cell percentage and time since MM diagnosis, duration of therapy, exposure to specific therapies (including cyclophosphamide, bortezomib, or lenalidomide), or bone marrow plasma cell content at time of apheresis. However, in the post-ind cohort, there was a trend of toward lower percentage early-mem phenotype (29% vs 49%, p=0.07) and lower CD4/CD8 ratio (median 1.4 vs 2.7, p=0.04) among patients who required >2 lines of therapy prior to apheresis (n=3) compared to the rest of the cohort (n=35).

Conclusion:

In MM patients, frequency of the early-mem T cell phenotype, a functionally validated biomarker of fitness for CAR T cell manufacturing, was significantly higher in leukapheresis products obtained after induction therapy compared to the relapsed/refractory setting, as was CD4/CD8 ratio and magnitude of in vitro T cell expansion. This result suggests that CAR T cells for MM would yield better clinical responses at early points in the disease course, at periods of relatively low disease burden and before exposure to multiple lines of therapy.

### Example 9: PD-1 Inhibitor Combinations as Salvage Therapy for Relapsed/Refractory Multiple Myeloma (MM) Patients Progressing after BCMA-Directed CART Cells

Background:

Autologous T cells expressing a chimeric antigen receptor (CAR) specific for B-cell maturation antigen (CART-BCMA cells) show activity in refractory MM, but relapses remain common. Anti-PD-1 antibodies (Abs) augment CART cell activity pre-clinically, and induced CART cell re-expansion and responses in DLBCL patients progressing after CD19-specific CART cells (Chong et al, Blood 2017, herein incorporated by reference in its entirety). The IMiDs lenalidomide (len) and pomalidomide (pom) may enhance efficacy, but also toxicity, of both CART cells and PD-1 inhibitors in MM. Elotuzumab (do) has clinical anti-MM activity in combination with IMiDs and dexamethasone (dex), and synergizes with anti-PD-1 Ab in pre-clinical models.

Methods:

Outcomes of 25 subjects enrolled in a phase 1 study of CART-BCMA cells in relapsed/refractory MM were previously described (Cohen et al, ASH 2017, #505, herein incorporated by reference in its entirety). Five subjects were identified and retrospectively reviewed who progressed after CART-BCMA and received a PD-1 inhibitor (pembrolizumab (pembro)) combination as their next therapy. Responses were assessed by IMWG criteria. CART-BCMA levels were assessed by flow cytometry and qPCR pre-treatment, 2-4 weeks after first pembro dose, then q4 weeks until progression. Pembro dosing was 200 mg every 3 weeks; dex dosing was 20-40 mg/week.

Results:

Characteristics of five subjects are shown in Table 39. Median prior lines were 9; all had high-risk cytogenetics. All were refractory to pom, 2 to pembro/pom/dex, and 1 to do. Best response to CART-BCMA was PR in 2, MR in 2, and PD in 1. Median time from CART-BCMA to pembro-based therapy was 117 days. All patients still had CART-BCMA cells detectable by qPCR, with 2 (pts. 07 and 21) still detectable by flow, at initiation of salvage therapy. The first pt. (02) received pembro/pom/dex and had MR but progressed at 2 months, with no detectable CART-BCMA re-expansion. The second pt. (07) had rapidly-progressing kappa light chain MM 2 months post-CART-BCMA and had previously progressed on pembro/pom/dex. He started elo/pembro/pom/dex and had MR at day 12 (free kappa 1446 to 937 mg/L), associated with robust expansion of CART-BCMA cells (875.64 to 20505.07 copies/μg DNA by qPCR; 0.7% to 6.4% of peripheral CD3+ cells by flow). Re-expanded CART-BCMA cells were predominantly CD8+ and highly activated (89% HLA-DR+, up from 18% pre-therapy). This response was short-lived, however, with progression 1 week later, and return of CART-BCMA levels to baseline at week 5. Three subsequent subjects then received elo/pembro/dex with either len or pom; with 2 MR and 1 SD, and PFS of 3 to 4 months. None had re-expansion of CART-BCMA cells. Non-specific immune modulation was observed and included altered CD4:CD8 T cell ratio (n=5), increased NK cell/decreased T cell frequency (n=4), and HLA-DR upregulation on CAR-negative T cells (n=2). More detailed phenotyping of CART and other immune cells, including PD-1 expression, is ongoing. With regard to toxicity, pt. 02 had self-limiting low-grade fevers and myalgias 4 weeks after pembro/pom/dex, associated with mild elevation in ferritin/CRP, suggestive of mild CRS. No other CRS was noted, including pt. 07 despite CART-BCMA re-expansion. One patient (17) developed recurrent expressive aphasia starting 2 months after elo/pembro/pom/dex, without signs of CRS and no observed expansion of CART-BCMA cells in blood or CSF. This resolved with stopping therapy and brief steroid taper.

Conclusions:

This study demonstrates that a PD1-inhibitor combination can induce CART cell re-expansion and anti-MM response in a MM patient progressing after CART-BCMA therapy. Since this patient previously progressed on pembro/pom/dex, the observed clinical activity was likely related to the CART cells, with elotuzumab also possibly contributing. This proof-of-principle observation suggests that a subset of patients may respond to checkpoint blockade or other immune-modulating approaches following BCMA CART cell therapy, meriting further study.

TABLE 39
Patient characteristics

| | | | Best | CART D 0 to | | Best | PFS post- | CART | Peak CART |
|---|---|---|---|---|---|---|---|---|---|
| | | | CART | Pembro D 0 | | Pembro | Pembro | qPCR | qPCR |
| Pt | Age/Sex | Cohort | Response | (Days) | Pembro Regimen | Response | (Days) | pre-Pembro\hat{ } | post-Pembro\hat{ } |
| 2 | 68/M | 1 | MR | 117 | Pembro/Pom/Dex | MR | 67 | 331.59 | 128.82 |
| 7 | 44/M | 1 | PR | 51 | Elo/Pembro/Pom/Dex | MR | 18 | 875.64 | 20505.07 |
| 13 | 59/M | 2 | MR | 149 | Elo/Pembro/Len/Dex | MR | 105 | 50.34 | 50.72 |
| 17 | 64/F | 3 | PR | 134 | Elo/Pembro/Pom/Dex | MR | 130 | 405.56 | 320.61 |
| 21 | 73/M | 3 | PD | 34 | Elo/Pembro/Len/Dex | SD | 83 | 8372.96 | 433.28 |

Cohort 1==5 × 10⁸ CART-BCMA cells alone;
Cohort 2 = 1.5 g/m² Cy and 5 × 10⁷ cells;
Cohort 3 = 1.5 g/m² Cy and 5 × 10⁸ cells Pembro = Pembrolizumab;
Elo = Elotuzumab; Pom = Pomalidomide;
Len = Lenalidomide; Dex = Dexamethasone;
Cy = cyclophosphamide SD = stable disease;
MR = minimal response;
PR = partial response;
PD = progressive disease;
PFS = progression-free survival;
units are copies/μg genomic DNA

### Example 10: Clinical and Biologic Activity of B-Cell Maturation Antigen-Specific Chimeric Antigen Receptor T Cells (CART-BCMA) in Refractory Multiple Myeloma: A Single-Centre, Open-Label, Phase 1 Trial

### Abstract

Background:

Chimeric antigen receptor (CAR) T cells are a promising new therapy for hematologic malignancies. B-cell maturation antigen (BCMA) is a cell-surface receptor with expression largely restricted to plasma cells, making it a rational target for multiple myeloma (MM) therapy. Methods: A phase I study of autologous T cells lentivirally-transduced with a novel, fully human, BCMA-specific CAR containing CD3t and 4-1BB signaling domains (CART-BCMA), were conducted in subjects with relapsed/refractory MM. Twenty-five subjects were treated in 3 dose cohorts: 1) 1-5×10⁸ CART-BCMA cells alone; 2) Cyclophosphamide (Cy) 1.5 g/m²+1-5×10⁷ CART-BCMA cells; and 3) Cy 1.5 g/m²+1-5×10⁸ CART-BCMA cells. No pre-specified BCMA expression level was required. Findings: Subjects had a median of 7 prior therapy lines; 96% were dual-refractory to a proteasome inhibitor and immunomodulatory drug; 96% had high-risk cytogenetics. CAR T cells were successfully manufactured in all cases. Toxicities included cytokine release syndrome and neurotoxicity, which were grade ¾ in 8 (32%) and 3 (12%) subjects, respectively, and reversible. CART-BCMA cells expanded in all subjects, most consistently in cohort 3. Clinical responses were seen in 4/9 (44%) in cohort 1, ⅕ (20%) in cohort 2, and 7/11 (63%) in cohort 3, including 5 partial, 5 very good partial, and 2 complete responses. Three subjects had ongoing responses at 11, 14, and 32 months. Decreased BCMA expression on residual MM cells was noted in responders; expression increased at progression in most. Responses were associated with CART-BCMA expansion, which was associated with CD4:CD8 T cell ratio and frequency of CD45RO−CD27+CD8+ T cells in the pre-manufacturing leukapheresis product. Interpretation: CART-BCMA infusions given with or without lymphodepleting chemotherapy are clinically active in heavily-pretreated MM patients and represent a novel approach to MM therapy.

This study provides further clinical validation of BCMA as a rational target for myeloma therapy, and characterises the safety and efficacy of a novel, fully human BCMA-specific CAR construct containing a 4-1BB co-stimulatory domain. This study further describes biologic and clinical activity of BCMA-specific CAR T cells both with and without lymphodepleting chemotherapy; demonstrates dynamic BCMA expression on myeloma cells post-infusion, particularly in responding patients; and identifies potential novel biomarkers of CAR T cell expansion and clinical response.

### Methods

### Study Design and Participants

Eligible subjects had relapsed and/or refractory MM after at least 3 prior regimens, or 2 prior regimens if dual-refractory to a proteasome inhibitor (PI) and immunomodulatory drug (IMiD). Other key eligibility criteria at screening included ECOG (Eastern Cooperative Oncology Group) performance status of 0-2; serum creatinine ≤2.5 mg/dL or estimated creatinine clearance ≥30 ml/min; absolute neutrophil count ≥1000/μl and platelet count ≥50,000/μl (≥30,000/μl if bone marrow plasma cells were ≥50% of cellularity); SGOT ≤3 times upper limit of normal and total bilirubin ≤2.0 mg/dl; left ventricular ejection fraction ≥45%; lack of active auto-immune disease; and lack of central nervous system involvement with myeloma. No pre-specified level of BCMA expression on MM cells was required.

This clinical trial (NCT02546167) was a phase 1, single-centre, open-label study. Initially a standard 3+3 dose-escalation design was used, exploring 3 sequential cohorts: 1) 1-5×10⁸ CART-BCMA cells alone; 2) Cyclophosphamide (Cy) 1.5 g/m²+1-5×10⁷ CART-BCMA cells; and 3) Cy 1.5 g/m²+1-5×10⁸ CART-BCMA cells. The protocol was amended to expand each cohort to 9 treated subjects, to gain more information about the safety and efficacy of CART-BCMA cells both with and without lymphodepleting conditioning and at a higher (1-5×10⁸) and lower (1-5×10⁷) dose. A subsequent amendment stopped enrollment in cohort 2 after 5 subjects due to suboptimal efficacy, and allowed up to 13 subjects in cohort 3; however, funding limitations ultimately ended enrollment after 11 treated Cohort 3 subjects (total n=25 treated).

### Procedures

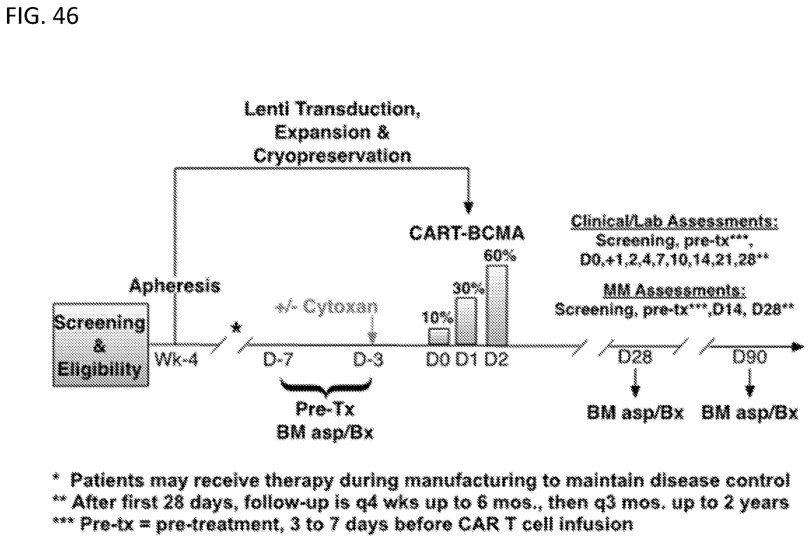

After a 2-week washout from therapy (4 weeks for monoclonal antibodies), subjects underwent steady-state leukapheresis to collect T cells for CART-BCMA manufacturing. Anti-myeloma therapy could resume during manufacturing until 2 weeks prior to first CART-BCMA infusion. CART-BCMA cells were administered intravenously over 3 days (10% of dose on day 0; 30% on day 1, and 60% on day 2), as described in Porter D L, et al., Sci Transl Med 2015; 7(303): 303ra139. The 30% or 60% dose could be held if subjects developed signs of CRS. Cy was administered 3 days prior to first CART-BCMA infusion. Clinical and laboratory assessments were performed as per FIG. 46.

CART-BCMA cells were manufactured in the FACT-accredited Clinical Cell and Vaccine Production Facility at the University of Pennsylvania, as described in Porter D L, et al., Sci Transl Med 2015; 7(303): 303ra139; Kalos M, et al., Sci Transl Med 2011; 3(95): 95ra73. The frequency of CD3, CD4, and CD8 cells was determined by flow cytometry in the seed culture at beginning and end of manufacturing. Fold expansion and population doubling of seeded cells was measured by cell counting on a Coulter Multisizer™. After manufacturing and quality control testing, CART-BCMA cells were cryopreserved until time of infusion.

Data on adverse events (AEs) were collected from time of first CART-BCMA infusion (or Cy administration for cohorts 2 and 3). Toxicity grade was determined according to National Cancer Institute's Common Terminology Criteria for Adverse Events version 4.0, with the exception of cytokine release syndrome, which was graded as per the University of Pennsylvania CRS Grading System (Table 38) (Porter D L, et al., Sci Transl Med 2015; 7(303): 303ra139). Myeloma responses were assessed by updated International Myeloma Working Group criteria (Kumar S, et al., Lancet Oncol 2016; 17(8): e328-46).

Research sample processing, freezing, and laboratory analyses were performed in the Translational and Correlative Studies Laboratory at the University of Pennsylvania, as described (Maude S L, et al., N Engl J Med 2014; 371(16): 1507-17; Porter D L, et al., Sci Transl Med 2015; 7(303): 303ra139; Kalos M, et al., Sci Transl Med 2011; 3(95): 95ra73). CART-BCMA cells were quantified from peripheral blood or bone marrow samples by flow cytometry and quantitative PCR. Reagents and protocols for flow cytometry are described in the Supplementary Methods. Genomic DNA was isolated directly from whole blood or marrow aspirate, and qPCR analysis was performed using ABI TaqMan technology and a validated assay to detect the integrated CAR transgene sequence as per Supplementary Methods and described in Kalos M, et al., Sci Transl Med 2011; 3(95): 95ra73.

Serum cytokine levels were assessed on batched cryopreserved samples using the human cytokine magnetic 30-plex panel (LHC6003M) from Life Technologies, as described in Maude S L, et al., N Engl J Med 2014; 371(16): 1507-17. Measurement of soluble BCMA, BAFF, and APRIL concentrations in serum was performed by ELISA using antibody sets for human BCMA (DY193), APRIL (DY884B) and BAFF (DT124-05) from R&D Systems. See Supplementary Methods for details.

Flow cytometric assessment of MM cells, including BCMA expression, on fresh bone marrow aspirate material was adapted from the EuroFlow protocol as described in Flores-Montero J, et al., Leukemia 2017; 31(10): 2094-103 and in Supplementary Methods. Flow cytometric assessment of T cell phenotype in cryopreserved leukapheresis specimens was performed and analyzed as described in Fraietta J A, et al., Nat Med 2018; 24(5): 563-71.

### Outcomes

The primary objective was to evaluate the safety of CART-BCMA in patients with relapsed/refractory myeloma. The primary endpoint was incidence of study-related grade 3 or higher AEs, including dose-limiting toxicities (DLTs). A DLT was defined as a serious AND unexpected AE whose relationship to study therapy could not be ruled out, occurring within 4 weeks of receiving protocol therapy. Hematologic toxicity was not considered a DLT due to the refractory nature of the underlying disease and expected myelosuppression from cyclophosphamide. In addition, any death related to protocol treatment, as well as any expected grade 4 organ toxicity or grade 4 neurologic toxicity that did not resolve or improve to grade 2 or less within 4 weeks of onset, despite medical management, was also considered a DLT. Secondary objectives were to assess feasibility of manufacturing CART-BCMA cells, and clinical activity. Secondary endpoints were frequency of successful manufacturing, and clinical outcomes, including response rates, progression-free survival, and overall survival. Exploratory endpoints included CART-BCMA expansion and persistence in vivo; changes in concentration of serum cytokines and soluble BCMA, and expression of BCMA on MM cells.

### Statistical Analysis

Statistical analysis of the study is primarily descriptive due to the pilot nature of the trial and small sample size of each cohort. Kaplan-Meier method was used to estimate duration of response, progression-free and overall survival and the associated median survival times. Association between a binary endpoint (e.g. response) and a continuous factor (e.g. CAR T cell numbers) was evaluated using the Mann-Whitney test. Association between a binary endpoint and a categorical factor (e.g. presence or absence of deletion 17p) was evaluated using Fisher's Exact test. Spearman correlations were used to measure the correlations between two continuous variables. Significance of the Spearman correlation against the null hypothesis of no correlation was computed based on permutation. Because the statistical analyses performed here are exploratory and hypothesis-generating in nature, no adjustment of the p-values was made for multiple comparisons. Exact p-values are reported when applicable. Analysis was performed using Graphpad Prism version 6.0.

### Results

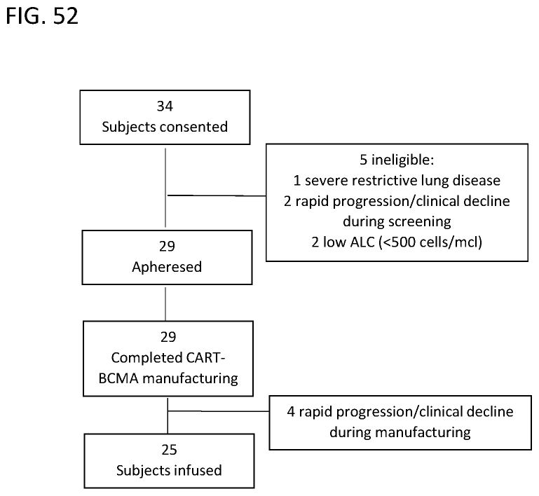

From November 2015-December 2017, 34 subjects consented and 29 were eligible and commenced manufacturing, with 25 receiving CART-BCMA infusions. Four were not treated due to rapid disease progression/clinical deterioration during manufacturing and bridging therapy (FIG. 52). Baseline characteristics and prior lines of therapy are summarized in Table 40, with individual details shown in Table 42. Subjects had a median of 7 prior lines of therapy, with 96% dual refractory to a proteasome inhibitor (PI) and immunomodulatory drug (IMID), 72% refractory to daratumumab, and 44% penta-refractory to 2 PIs, 2 IMIDs, and daratumumab. Ninety-six % had at least 1 high-risk cytogenetic abnormality; 68% had either deletion 17p or a TP53 mutation. Baseline tumor burden was high (median 65% myeloma cells on bone marrow biopsy), and 28% had extramedullary disease.

TABLE 40
Subject characteristics

| Characteristic (n = 25) | Median (range) or % | |
|---|---|---|
| Age | 58 | (44-75) |
| Gender | 68% male; 32% female | |
| Median time from diagnosis (years) | 4.6 | (1.8-14.5) |
| High-risk cytogenetics* | 96% | |
| Del 17p or TP53 mutation | 68% | |
| Prior lines of therapy | 7 | (3-13) |
| Len/Bort/Pom/Carf**/Dara (% exposed) | 100%/100%/92%/96%/76% | |
| Len/Bort/Pom/Carf/Dara (% refractory) | 76%/88%/88%/80%/72% | |
| % Dual-/% Quad-/% Penta-refractory | 96%/56%/44% | |
| Prior Autologous SCT | 92% | |
| Extramedullary disease | 28% | |
| % Bone marrow plasma cells | 65 | (0-95) |
| Absolute CD3+ T cell count (cells/μL) | 538 | (151-1529) |
| pre-leukapheresis*** | | |
| Day 0 LDH (units/L) | 175 | (75-385) |
| Day 0 serum creatinine (mg/dL) | 0.93 | (0.55-2.87) |
| Day 0 hemoglobin (g/dL) | 9.1 | (6.4-12.4) |
| Day 0 platelets (×10³/μL) | 128 | (13-316) |

*Includes complex karyotype, gain 1q, deletion 17p, t(14; 16), and/or t(4; 14).
**Includes 1 patient who received oprozomib.
***n = 23 (subjects 01 and 02 did not have pre-pheresis T cell counts done).
Normal range = 900-3245 cells/μL
Bort = bortezomib; Carf = carfilzomib; Dara = daratumumab; Del = deletion; Dual-refractory = refractory to both a proteasome inhibitor (PI) and immunomodulatory agent (IMiD); LDH = lactate dehyrdrogenase; Len = lenalidomide; Penta-refractory = refractory to 2 Pls, 2 IMiDs, and dara; Pom = pomalidomide; Quad-refractory = refractory to 2 Pls and 2 IMiDs; SCT = stem cell transplant.

All subjects successfully manufactured minimum target goal of CART-BCMA cells, though 1 subject required 2 leukaphereses and manufacturing attempts. Final products were comprised of a median of 97% CD3+ T cells, with median CD4/CD8 ratio of 1.7. Twenty-one subjects received all 3 planned CART-BCMA infusions, with 4 receiving 40% of planned dose (3^{rd} infusion held due to early CRS). Further details of manufacturing, product characteristics, and dosing for each subject are shown in Table 43.

Grade 3 or higher adverse events, regardless of attribution, were seen in 24/25 subjects (96%) and are summarized in Table 41, with individual adverse events for each subject listed in Table 44. Cytokine release syndrome (CRS) was observed in 22/25 subjects (88%), and was grade ¾ on Penn grading scale (Porter D L, et al., Sci Transl Med 2015; 7(303): 303ra139) (Table 38) in 8 (32%), all of whom were treated at the 1-5×10⁸ dose. Median time to CRS onset was 4 days (range 1-11), with a median duration of 6 days (range 1-18), and median duration of hospitalization of 7 days (range 0-40). CRS was associated with elevations in ferritin and C-reactive protein, as described previously (Maude S L, et al., N Engl J Med 2014; 371(16): 1507-17). Seven subjects (28%) received IL-6 blockade with either tocilizumab (n=6) or siltuximab (n=1).

TABLE 41
Grade 3 or higher adverse events, regardless of attribution

| | Grade 3 | Grade 4 | Grade 5 | All | |
|---|---|---|---|---|---|
| Event | n | n | n | n (%) | |
| Leukopenia | 3 | 8 | 0 | 11 | (44%) |
| Neutropenia | 2 | 9 | 0 | 11 | (44%) |
| Lymphopenia | 3 | 6 | 0 | 9 | (36%) |
| Cytokine release syndrome | 7 | 1 | 0 | 8 | (32%) |
| Thrombocytopenia | 3 | 4 | 0 | 7 | (28%) |
| Hypophosphatemia | 7 | 0 | 0 | 7 | (28%) |
| Febrile neutropenia | 6 | 0 | 0 | 6 | (24%) |
| Hyponatremia | 6 | 0 | 0 | 6 | (24%) |
| Anemia | 4 | 1 | 0 | 5 | (20%) |
| Hypocalcemia | 3 | 2 | 0 | 5 | (20%) |
| AST increased | 2 | 1 | 0 | 3 | (12%) |
| Hypokalemia | 2 | 1 | 0 | 3 | (12%) |
| Neurotoxicity* | 1 | 2 | 0 | 3 | (12%) |
| Hypertension | 3 | 0 | 0 | 3 | (12%) |
| Lung infection | 2 | 0 | 0 | 2 | (8%) |
| Hypoalbuminemia | 2 | 0 | 0 | 2 | (8%) |
| Supraventricular tachycardia | 1 | 0 | 0 | 1 | (4%) |
| Papilledema | 1 | 0 | 0 | 1 | (4%) |
| Abdominal pain | 1 | 0 | 0 | 1 | (4%) |
| Death NOS** | 0 | 0 | 1 | 1 | (4%) |
| Fatigue | 1 | 0 | 0 | 1 | (4%) |
| Infections - other (fungemia) | 0 | 1 | 0 | 1 | (4%) |
| Sepsis | 0 | 1 | 0 | 1 | (4%) |
| Urinary tract infection | 1 | 0 | 0 | 1 | (4%) |
| Alk phos increased | 1 | 0 | 0 | 1 | (4%) |
| Ejection fraction decreased | 1 | 0 | 0 | 1 | (4%) |
| Fibrinogen decreased | 1 | 0 | 0 | 1 | (4%) |
| Hyperglycemia | 0 | 1 | 0 | 1 | (4%) |
| Hypoglycemia | 1 | 0 | 0 | 1 | (4%) |
| Pleural effusion | 1 | 0 | 0 | 1 | (4%) |
| Pleural hemorrhage | 0 | 1 | 0 | 1 | (4%) |

Highest grade toxicity experienced by subject is reported in table.
n = number of subjects who had the event; Alk phos = alkaline phosphatase; AST = aspartate aminotransferase.
*Neurotoxicity includes 1 subject with grade 3 seizures, grade 4 delirium, and grade 4 reversible posterior leukoencephalopathy syndrome (RPLS) (also known as posterior reversible encephalopathy syndrome (PRES)); 1 subject with grade 3 delirium and grade 4 encephalopathy; and 1 subject with grade 3 encephalopathy.
**Death NOS (not otherwise specified) in subject 08 with candidemia and rapidly progressive myeloma; family chose to pursue comfort measures only.

Neurotoxicity was seen in 7/25 subjects (28%), and was mild (grade 1/2) in 4 (transient confusion and/or aphasia). Three (12%) had grade 3/4 encephalopathy including 1 subject (03) in cohort 1 with a DLT of PRES (posterior reversible encephalopathy syndrome) with severe obtundation, recurrent seizures and mild cerebral edema on MRI (magnetic resonance imaging) that fully resolved after treatment with high-dose methylprednisolone (1 g/day x 3) and cyclophosphamide 1.5 g/m². The others had no objective changes on MRI. All 3 subjects with severe neurotoxicity had high tumor burden (2 with extramedullary disease); had received a dose of 5×10⁸ CART-BCMA cells; and had grade 3 or 4 CRS. The only other DLT was grade 3 cardiomyopathy and grade 4 pleural hemorrhage/spontaneous hemothorax in subject 27 (cohort 3) in the setting of CRS, coagulopathy, thrombocytopenia, and extensive myelomatous rib lesions; all these toxicities fully resolved. One subject (08) who had grade 4 encephalopathy and CRS initially improved with tocilizumab and steroids, then developed candidemia and progressive myeloma/evolving plasma cell leukemia. Family opted for comfort care and he expired at day 24. No other deaths occurred on study. No unexpected off-target toxicities were observed.

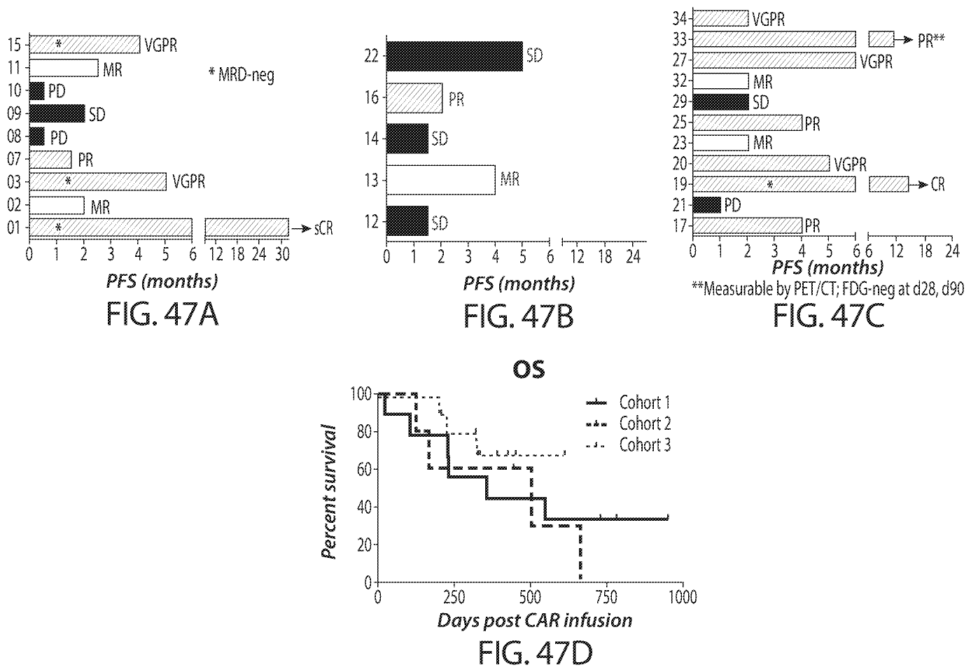

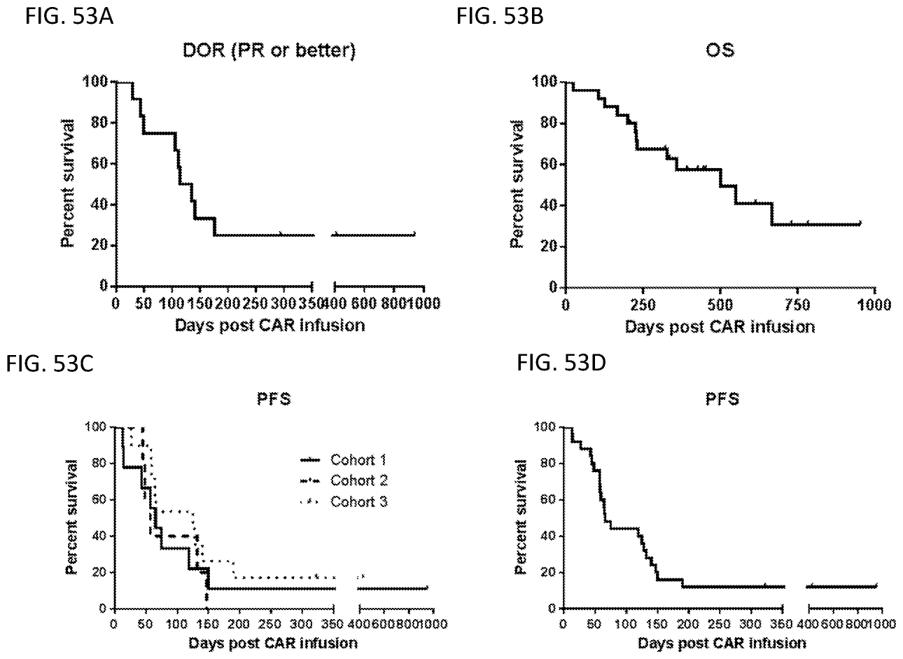

Regarding clinical outcomes, objective responses (partial response (PR) or better) were confirmed in 4/9 subjects (44%) in cohort 1, 1/5 (20%) in cohort 2, and 7/11 (63%) in cohort 3 (FIGS. 47A-47C), including 5 PR, 5 very good partial responses (VGPR), 1 complete response (CR), and 1 stringent complete response (sCR). Thus overall response rate was 12/25 (48%), and was 11/20 (55%) at the more effective dose of 1-5×10⁸ CART-BCMA cells. Five additional subjects had minimal response (MR). Four of 7 subjects with extramedullary disease responded (FIG. 32B). Four subjects (01, 03, 15, 19) had no detectable myeloma (i.e. MRD-negative) by flow cytometry (estimated sensitivity 10⁻⁵) from post-infusion marrow aspirates at months 1 and/or 3. Median time to first response was 14 days. Based on Kaplan-Meier estimates, median duration of response (for PR or better) was 124.5 days (range 29-939+) (FIG. 53A). At time of data cut-off, 3 subjects (01, 19, 33) remained progression free at 953, 427, and 322 days (roughly 32, 14, and 11 months), respectively. All other subjects have progressed, and median progression-free survival (PFS) is 65, 57, and 125 days for cohorts 1, 2, and 3, respectively (FIG. 53C). At time of data cut-off, 13 subjects had expired, with median overall survival of 502 days for all subjects (FIG. 53B), and 359 days, 502 days, and not reached for cohorts 1, 2, and 3, respectively (FIG. 47D).

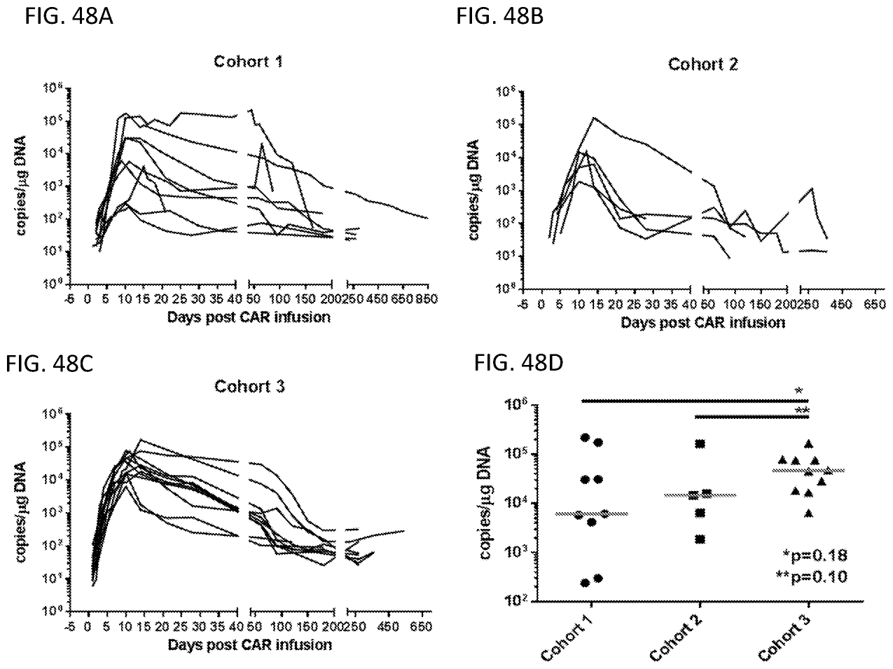

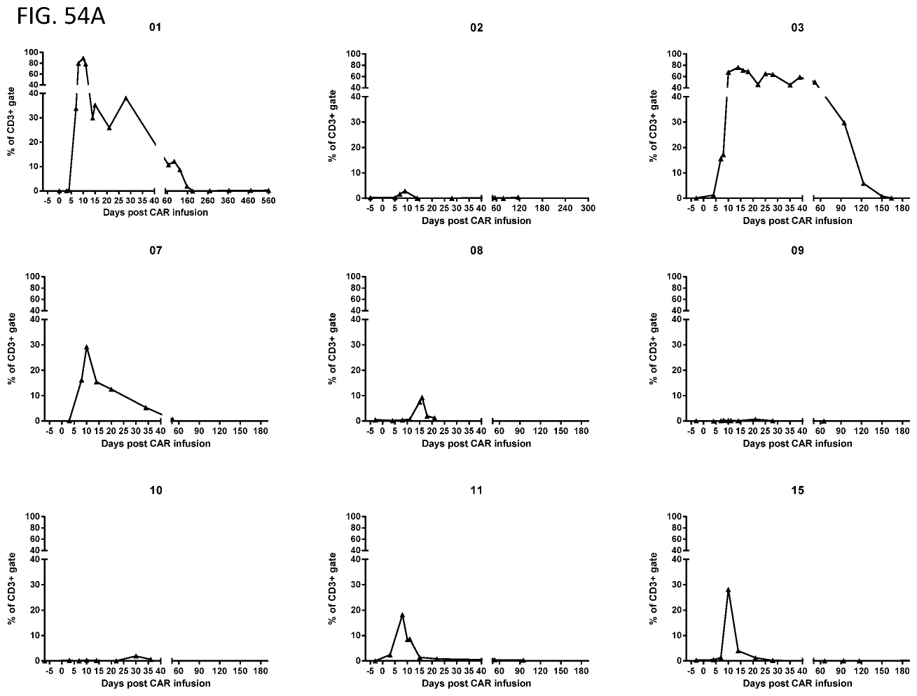

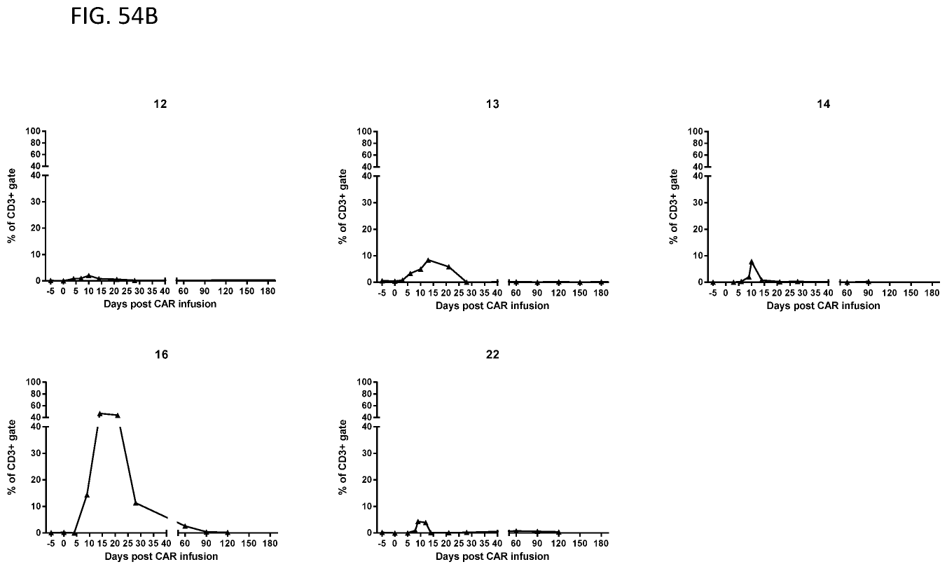

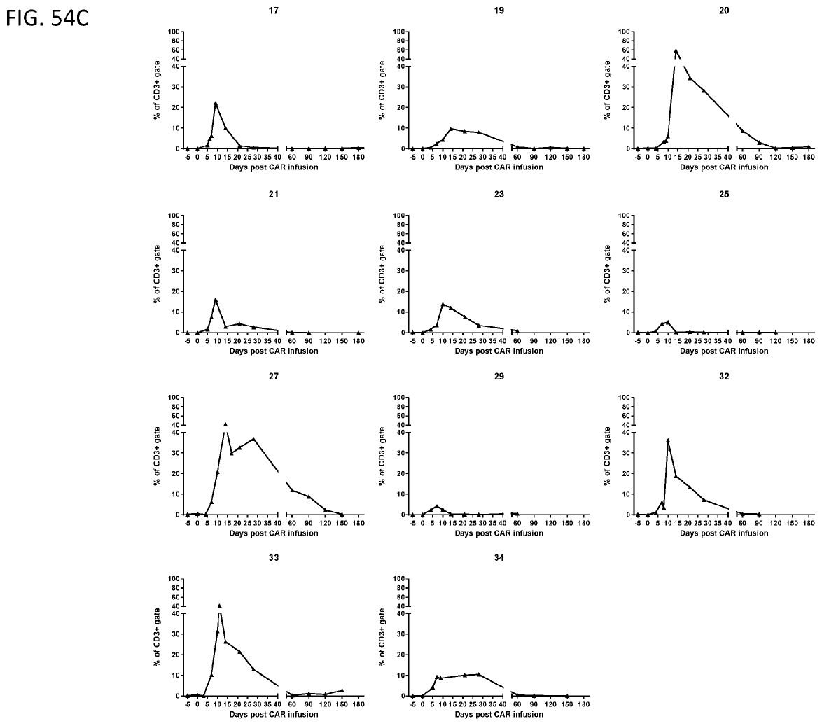

All infused subjects had detectable CART-BCMA cells in peripheral blood by qPCR (FIGS. 48A-48C), and 24/25 had detectable CAR⁺ T cells by flow cytometry (FIGS. 54A-54C; FIG. 38 for representative staining). Expansion generally peaked at day 10-14, and appeared most uniform in cohort 3 with Cy conditioning and the higher dose of CART-BCMA cells, while it was more heterogeneous in cohorts 1 and 2, though this difference did not meet statistical significance (FIG. 48D). Despite a predominance of CD4+ T cells in the infused product, CART-BCMA cells circulating in blood were predominantly CD8+, and were highly activated, with a median of 94% (range 21-94%) of CAR+CD3+ cells expressing HLA-DR during peak expansion (Table 45). CART-BCMA levels in marrow aspirates generally mirrored those in peripheral blood, and also were elevated in pleural fluid and cerebrospinal fluid for subject 03, and pleural fluid from subject 27, demonstrating widespread trafficking (Table 46). Following peak expansion, CART-BCMA cell levels by qPCR declined in a log-linear fashion in the majority of patients (FIGS. 48A-48C), and were still detectable at 3 months post-infusion in 20/20 (100%) subjects tested, and at 6 months in 14/17 (82%) tested. Subject 01 (in stringent CR) continued to have detectable cells by qPCR when last tested at 2.5 years post-infusion.

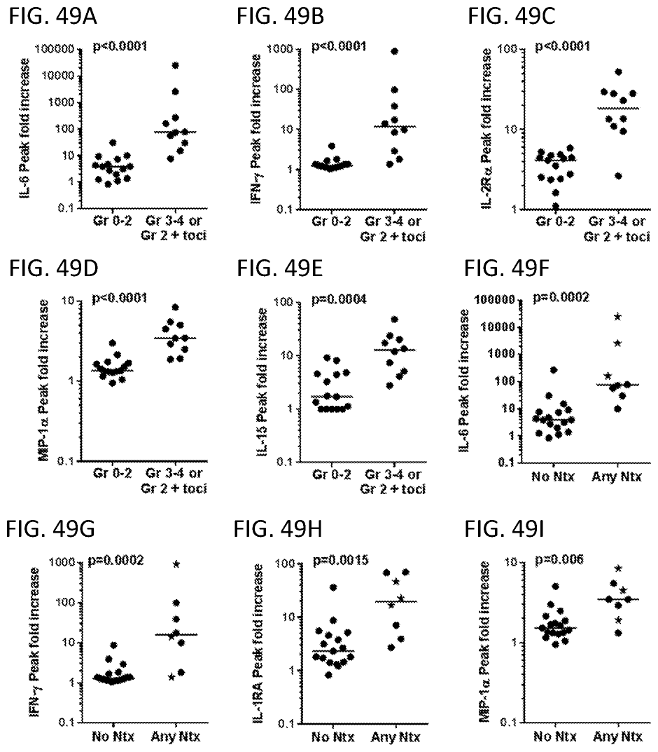

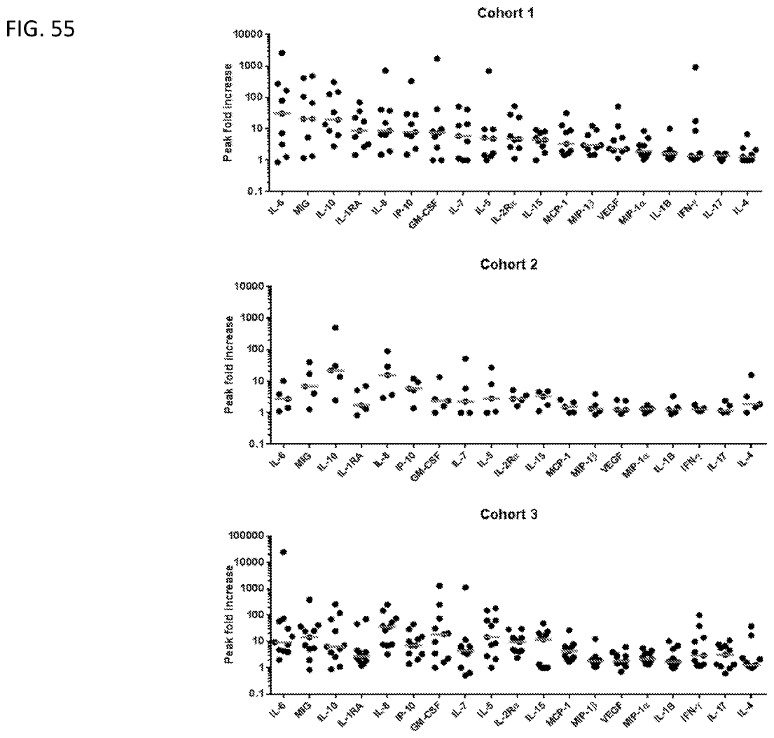

Thirty cytokines were quantified in peripheral blood serum before and after CART-BCMA infusion. Nineteen of these were increased >5-fold over baseline in more than 1 subject, with the most frequent increases observed for IL-6, IL-10, monokine induced by interferon-gamma (MIG, CXCL9), IP-10, IL-8, GM-CSF, and IL-1 receptor antagonist (IL-1RA) (FIG. 55). More severe CRS (Grade 3/4, or Grade 2 receiving tocilizumab) was associated with increases in multiple cytokines (Table 47), most significantly with IL-6, IFN-γ, IL-2 receptor alpha (IL2-Rα), macrophage inflammatory protein 1 alpha (MIP-1a), and IL-15 (FIGS. 49A-49E). Neurotoxicity was most strongly associated with increases in IL-6, IFN-γ, IL-1RA and MIP-1α, (FIGS. 49F-49I, see Table 48 for full analysis). Significant differences were not observed in peak fold cytokine increases between Cohorts 1 and 3, which received the same dose of CART-BCMA cells with or without Cy conditioning, respectively (FIG. 55).

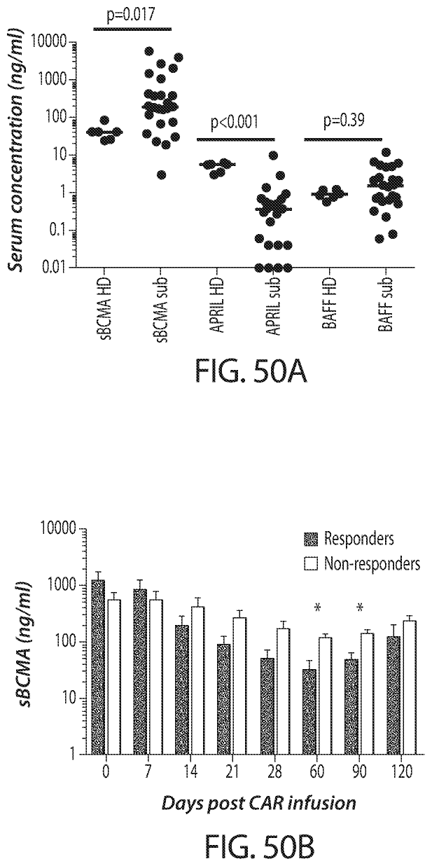

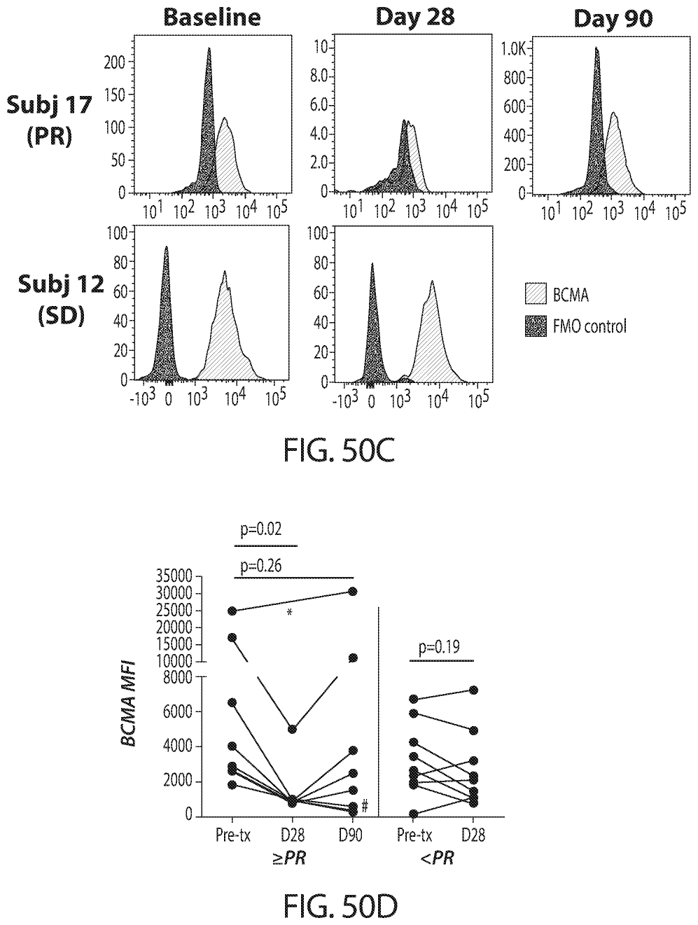

Serum concentration of sBCMA, as well as its ligands BAFF and APRIL, were also assessed. Compared to a panel of healthy donors (HD), enrolled subjects had significantly elevated sBCMA and reduced APRIL levels at baseline, with high variability amongst subjects (FIG. 50A). BAFF concentrations in subjects were not significantly different from HD. Serial assessments of serum sBCMA showed decreases following CART-BCMA infusions, which were more pronounced in responding compared to non-responding subjects (FIG. 50B), and which increased as subjects developed progressive disease, suggesting that serum sBCMA concentration may be a useful adjunctive biomarker for assessing myeloma disease burden. Twenty subjects were evaluable for BCMA surface expression on MM cells by flow cytometry performed on fresh marrow aspirates prior to treatment, and all had detectable BCMA expression, though intensity varied (median mean fluorescence intensity (MFI)=3741, range 206-24842; see FIG. 42 for representative gating). Of 18 subjects with evaluable serial BCMA expression (FIG. 50C), either at 1 month (n=16), 3 months (n=8), and/or 5.5 months (n=1), 12 (67%) had a decline in BCMA intensity at least at 1 post-infusion time-point, including 8/9 responders and 4/9 non-responders (FIG. 50D). BCMA intensity was lowest on residual MM cells 1 month post-CART-BCMA, and increased back toward baseline in most, but not all, subjects with subsequent testing. Individual subject details are provided in Table 49.

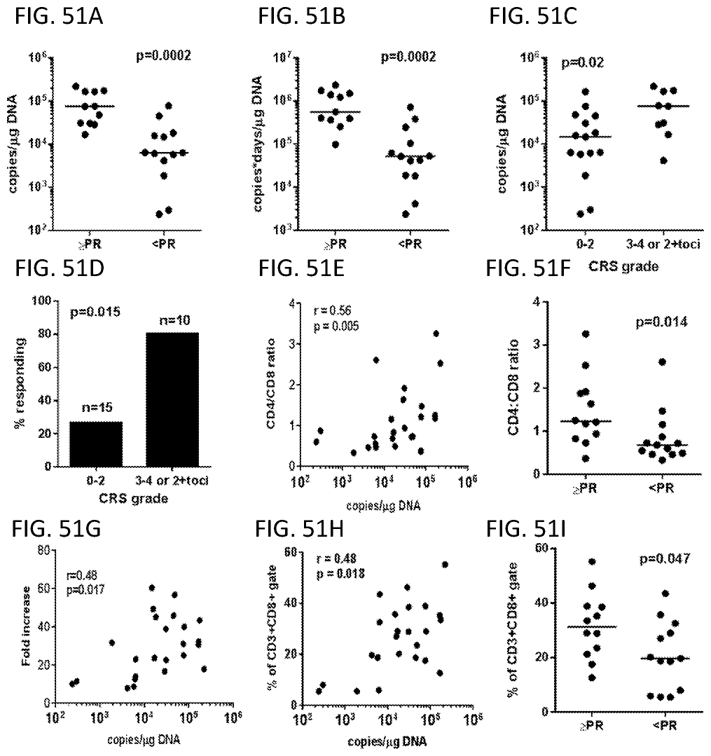

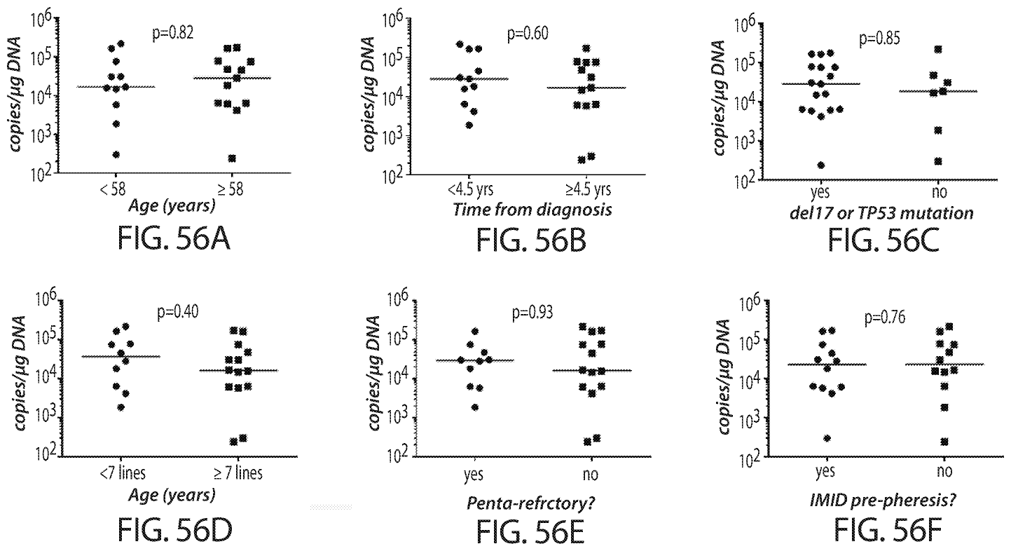

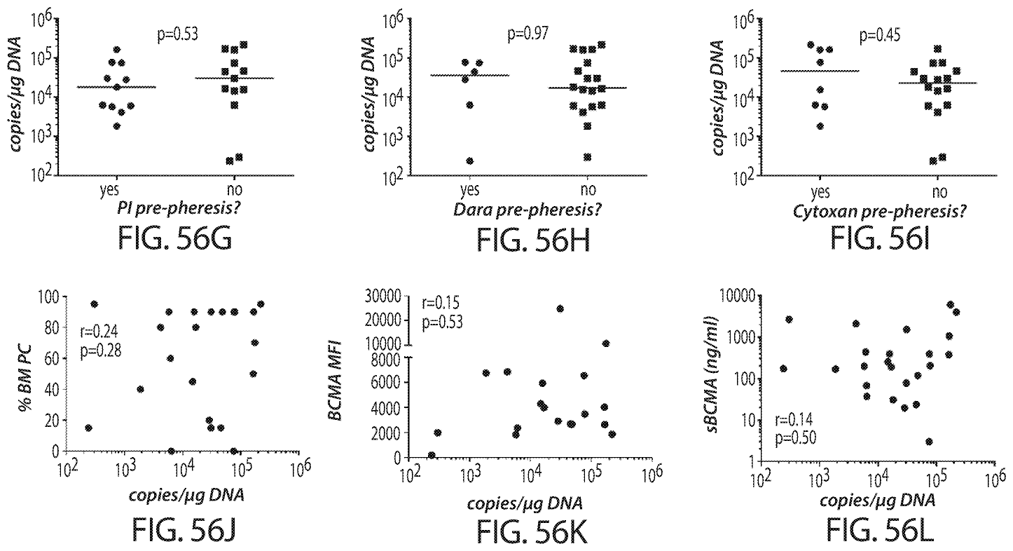

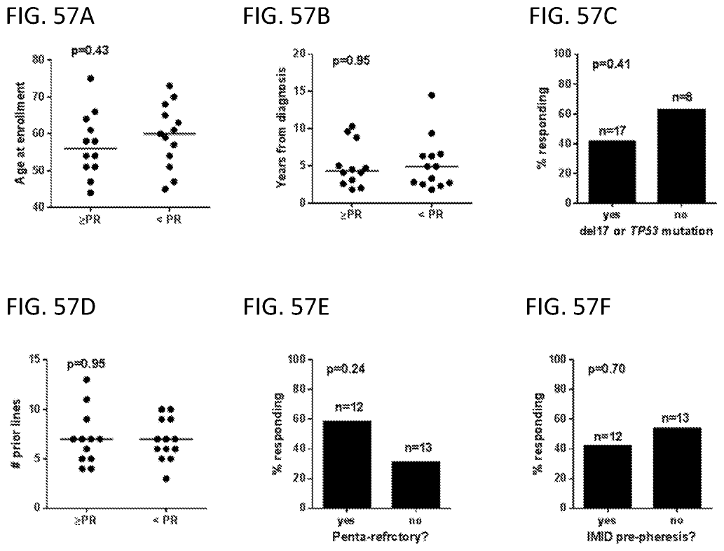

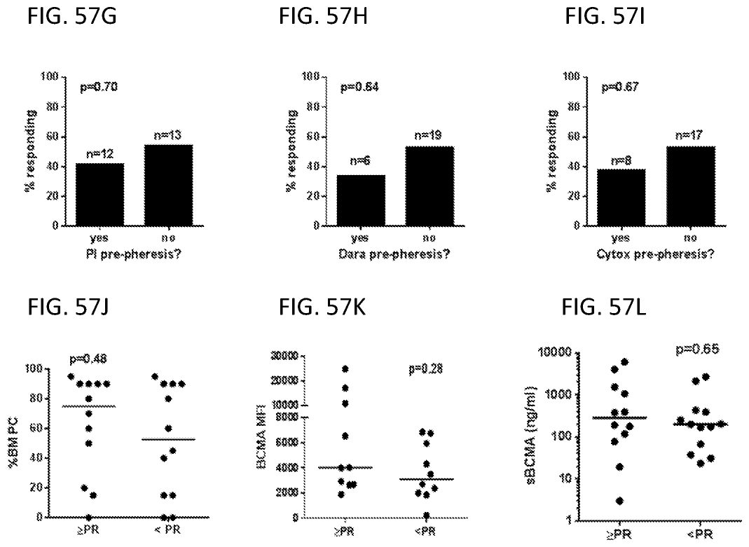

Responses were significantly associated with peak expansion by qPCR (median 75339 copies/μg DNA for ≥PR vs. 6368 copies/μg for ≤PR, p=0.0002), as well as with persistence over the first 28 days, as measured by the area under the curve (AUC₀₋₂₈d) (median 561796 copies*days/μg DNA for ≥PR vs. 52391 copies*days/μg DNA for <PR, p=0.0002) (FIGS. 51A-51B). Both expansion and response were more likely in the setting of more severe CRS (grade 3/4 or grade 2 requiring tocilizumab) (FIGS. 51C-51D). Neither expansion nor response were significantly associated with age, years from diagnosis, number of prior lines of therapy, presence of dell7p or TP53 mutation, being penta-refractory, most recent therapy pre-pheresis, bone marrow MM cell percentage, baseline serum sBCMA concentration, or MM cell BCMA intensity (FIGS. 56A-56L and 57A-57L).

In order to explore other pre-treatment characteristics potentially associated with expansion and/or response, features of the CART-BCMA product before, during, and at end of manufacturing were analyzed. It was found that a higher CD4/CD8 T cell ratio in the leukapheresis product, pre-manufacturing, was associated with greater in vivo CART-BCMA expansion (FIG. 51E), and to a lesser degree, response (FIG. 51F), while absolute CD3+, CD4+, or CD8+ T cell numbers in the leukapheresis product, or CD4/CD8 ratio in the final CART-BCMA product at end of manufacturing was not (data not shown). Fold expansion of seeded cells during manufacturing also correlated with in vivo CART-BCMA expansion (FIG. 51G), suggesting that in vitro proliferative capacity may predict for in vivo activity. Finally, CD8+ T cells within the leukapheresis products in subjects treated with CART-BCMA cells were examined and it was found that subjects with higher frequencies of CD27+CD45RO− CD8+ T cells were more likely to have robust in vivo expansion and clinical response (FIGS. 51H-51I).

### DISCUSSION

CAR T cell therapy is emerging as a promising therapeutic option for B-cell malignancies, with the potential for durable disease control following a single treatment, differentiating it from other therapies that require repeated and/or continuous administration. In this report, the potential of CAR T cell therapy was demonstrated in advanced refractory myeloma, with 12/25 subjects (48%) achieving a partial response or better, including 7/11 (63%) treated at optimal doses (>10⁸ CART-BCMA cells) following lymphodepleting chemotherapy. Three subjects had ongoing remissions >11 months after CART-BCMA therapy, including one ongoing sCR at 2.5 years. This is notable given the highly adverse biological features of the enrolled subjects' myeloma, including high tumor burden, rapidly progressing disease, and high-risk genetics. This clinical activity further validates BCMA as a highly attractive target in myeloma. Importantly, this activity was seen despite the fact that this study, unlike the previous study, did not exclude patients with low BCMA expression or high tumor burden, and used either no lymphodepletion or Cy alone, compared with Cy+fludarabine in the prior study (Ali S A, et al., Blood 2016; 128(13): 1688-700; Brudno J N, et al., J Clin Oncol 2018; 36(22): 2267-80). CAR T cell products were successfully manufactured from all subjects, despite baseline T cell lymphocytopenia, and engraftment was seen in all as well, though peak levels and persistence of CAR T cells varied significantly amongst subjects.

In this study, responses correlated with degree of in vivo expansion, which in turn was associated with higher pre-manufacturing CD4/CD8 T cell ratio, pre-manufacturing frequency of CD45RO−CD27+CD8+ T cells, and magnitude of in vitro proliferation during manufacturing. This suggests that more effective CART-BCMA products may be derived from subjects with a less differentiated, more naïve and/or stem cell memory-like T cell compartment. These data suggest that pre-treatment phenotypic and/or functional T cell characteristics may aid in the prediction of response to CART-BCMA therapy. They also suggest that treatment of patients earlier in the course of their disease, when T cells may be intrinsically “fitter,” or modifying manufacturing techniques to generate more phenotypically favorable CAR+ T cells, may be more effective.

Successful adoptive transfer of tumor-specific T cells, including CAR T cells, in humans has most commonly followed some form of lymphodepleting conditioning (Turtle C J, et al., Sci Transl Med 2016; 8(355): 355ra116; Maude S L, et al., N Engl J Med 2014; 371(16): 1507-17; Porter D L, et al., Sci Transl Med 2015; 7(303): 303ra139; Dudley M E, et al., J Clin Oncol 2008; 26(32): 5233-9), which has been demonstrated to enhance T cell-mediated anti-tumor immunity via multiple potential mechanisms, including reduction of cellular “sinks” leading to increased availability of homeostatic cytokines, and depletion of suppressor cell populations such as regulatory T cells, among others (Gattinoni L, et al., Nat Rev Immunol 2006; 6(5): 383-93). This study demonstrates that lymphodepletion is not absolutely required for robust and sustained CAR T cell expansion and clinical activity, as seen with subjects 01 and 03 in Cohort 1. However, short-term expansion was observed more consistently in Cohort 3, where subjects received Cy conditioning, compared to Cohort 1 (FIGS. 48A and 48C), demonstrating an effect of lymphodepletion on CAR-T cell kinetics following adoptive transfer. It is possible that modifying the lymphodepletion (e.g. adding fludarabine to Cy) may further augment the activity of CART-BCMA cells.

An important unanswered question for BCMA-targeted CAR T cells is whether there is a threshold of BCMA expression on MM cells required for optimal recognition and killing. In the previously-reported NCI trial, 52/85 (62%) of pre-screened bone marrow biopsies stained for BCMA by IHC met their pre-specified threshold for eligibility, meaning more than a third of potentially eligible MM patients would have been excluded (Ali S A, et al., Blood 2016; 128(13): 1688-700). This study did not require any specific level of BCMA as an eligibility requirement, and identified MM cell BCMA expression by flow cytometry in all subjects, consistent with recent data that flow cytometry is more sensitive than IHC for this purpose (Salem D A, et al., Leuk Res 2018; 71: 106-11). Baseline BCMA intensity by flow cytometry did not correlate with either expansion or response in this study (FIGS. 56A-56L and 57A-57L), suggesting that excluding patients based on baseline BCMA expression is likely not necessary.

The observed dynamics of BCMA surface expression on MM cells, with the residual MM cells from several subjects in this study having significantly diminished BCMA intensity following CAR T cell therapy (FIGS. 50A-50D), highlights an important area for future research into resistance to CART-BCMA therapy. Down-modulation of BCMA expression was also observed in at least 1 subject in the NCI study (Brudno J N, et al., J Clin Oncol 2018; 36(22): 2267-80), suggesting that it may be a common means of MM cell escape from BCMA-directed CAR-T cell therapies. Surface BCMA expression subsequently increased in most subjects upon progression, suggesting a transcriptional or post-translational mechanism, such as increased shedding from the cell surface. Alternatively, there may be immune selection for BCMA-dim/negative clonal variants, which are subsequently outcompeted by residual BCMA+clones upon loss of CART-BCMA cells. This suggests that most patients progressing after CART-BCMA will remain candidates for additional BCMA-targeted therapies.

The primary toxicities of CAR T cells remain cytokine release syndrome (CRS) and neurotoxicity. The frequency and severity of CRS in this study was similar to that reported in CD19-targeted CAR T cell trials (Maude S L, et al., N Engl J Med 2014; 371(16): 1507-17; Porter D L, et al., Sci Transl Med 2015; 7(303): 303ra139), and was abrogated with IL-6 receptor blockade therapy. Though patient numbers are small, peak serum cytokine increases did not appear to differ significantly with or without Cy lymphodepletion, when CAR T cell dose was kept the same (Cohort 1 vs. 3, FIG. 55). Interestingly, however, median peak fold-increases of IL-6 and several other cytokines (e.g. IFN-γ, IL-10, GMCSF, IL-17) in this study were 1 to 2 orders of magnitude lower than that reported in the NCI BCMA CAR T cell study (Brudno J N, et al., J Clin Oncol 2018; 36(22): 2267-80), despite a higher tumor burden in this study. One explanation for this difference may be the co-stimulatory domains used within the 2 CAR constructs, since CD28 domains, such as that used in the NCI CAR construct, have been associated with more rapid CAR T cell proliferation and cytokine release than 4-1BB domains (Milone M C, et al., Mol Ther 2009; 17(8): 1453-64), as used in this CAR construct. However, the small numbers of patients and multiple differences between the studies with regards to inclusion criteria, dose, schedule, and lymphodepletion regimen preclude definitive conclusions.

Neurotoxicity has been reported in up to 50% of subjects in some CAR T cell trials (Turtle C J, et al., Sci Transl Med 2016; 8(355): 355ra1 16; Turtle C J, et al., J Clin Invest 2016; 126(6): 2123-38; Kochenderfer J N, et al., J Clin Oncol 2015; 33(6): 540-9); can occur concurrently with or subsequent to CRS; often does not improve with tocilizumab; and is reversible in most, but not all, cases. Neurotoxicity has been associated with early onset of CRS and rapid elevation of inflammatory cytokines both within the serum and CNS, perhaps leading to increased CNS vascular permeability (Gust J, et al., Cancer Discov 2017).

Consistent with this, this study identified peak serum increases of IL-6, IFN-γ, and MIP-1α as most associated with neurotoxicity in this study (FIGS. 49A-49I). Interestingly, neurotoxicity was also associated with peak fold increase in IL-1RA, an endogenous inhibitor of the pro-inflammatory effects of IL-1α and IL-1β, which have been implicated in CART cell-associated neurotoxicity (Giavridis T, et al., Nat Med 2018; 24(6): 731-8; Norelli M, et al., Nat Med 2018; 24(6): 739-48). This perhaps reflects induction of an (ultimately ineffective) feedback mechanism in patients with neurotoxicity, and suggests that augmenting IL-1 blockade via the recombinant IL-1RA anakinra may have therapeutic benefit in this setting, as demonstrated in pre-clinical models (Giavridis T, et al., Nat Med 2018; 24(6): 731-8; Norelli M, et al., Nat Med 2018; 24(6): 739-48). This study demonstrating rapid reversal of a PRES-like syndrome in subject 03 suggests that cyclophosphamide may also be an option in steroid-refractory cases.

In summary, autologous T cells expressing a fully human BCMA-specific CAR could expand and induce objective responses, both with and without lymphodepleting chemotherapy, in subjects with advanced, refractory MM, and represent a promising new therapeutic approach. The toxicity profile appears similar to that seen with CD19-directed CAR T cells in B-cell malignancies. Challenges include disease progression during manufacturing, potential for antigen escape due to changes in BCMA expression, and durability of responses. Subsequent studies exploring less heavily-pretreated/refractory patient populations, dual-antigen-targeting CAR constructs, novel lymphodepletion regimens or manufacturing protocols, and off-the-shelf CART products may further optimize the safety and long-term efficacy of this approach.

### Supplementary Methods

Reagents and Protocols for Flow Cytometry:

Antibodies for CAR T cell detection panels were anti-CD45 V450 (clone HI30), anti-CD14 V500 (clone M5E2), anti-CD56 Ax488 (clone B159), anti-CD4 PerCP-Cy5.5 (clone RPA-T4), anti-CD8 APC-H7 (clone SK1) (all from BD Bioscience). Also, anti-CD3 BV605 (clone OKT3), anti-HLA-DR BV711 (clone L243), anti-CD19 PE-Cy7 (clone H1B19) were used from Biolegend. CART-BCMA expression was assessed by using a bis-biotinylated BCMA-Fc recombinant protein and the secondary staining reagent Streptavidin-PE from BD Bioscience (cat #554061). Cells were resuspended in 100 μL PBS containing 1% fetal bovine serum, 0.02% sodium azide and bis-biotinylated BCMA-Fc and incubated for 30 min on ice, washed, resuspended in 100 μL PBS containing 1% fetal bovine serum, 0.02% sodium azide, surface antibodies and SA-PE, and incubated for 30 minutes on ice, washed, resuspended in 250 ul PBS containing 1% fetal bovine serum and 0.02% sodium azide and acquired using a Fortessa flow cytometer equipped with a violet (405 nm), blue (488 nm), a green (532 nm), and a red (628 nm) laser. Data were analyzed using FlowJo software (Version 10, Treestar). Compensation values were established using eBioscience UltraComp eBeads (eBioscience cat #01-222-42) and DIVA software.

Quantitative PCR:

Genomic DNA was isolated directly from whole blood or marrow aspirate, and qPCR analysis was performed using ABI TaqMan technology and a validated assay to detect the integrated CAR transgene sequence as described in Kalos M, et al., Sci Transl Med 2011; 3(95): 95ra73 using triplicates of 200 ng of genomic DNA per time point for peripheral blood and marrow samples. To determine copy number per unit DNA, an eight-point standard curve was generated consisting of 5 to 106 copies of lentivirus plasmid spiked into 100 ng of non-transduced control genomic DNA. The number of copies of plasmid present in the standard curve was verified using digital qPCR with the same primer/probe set and performed on a QuantStudio 3D digital PCR instrument (Life Technologies). Each datapoint (sample and standard curve) was evaluated in triplicate with a positive Ct value in three of three replicates with percent coefficient of variation of less than 0.95% for all quantifiable values. To control for the quality of interrogated DNA, a parallel amplification reaction was performed using 20 ng of genomic DNA and a primer/probe combination specific for a non-transcribed genomic sequence upstream of the CDKN1A (p21) gene as described in Kalos M, et al., Sci Transl Med 2011; 3(95): 95ra73. These amplification reactions generated a correction factor to adjust for calculated versus actual DNA input. Copies of transgene per microgram of DNA were calculated according to the formula: copies per microgram of genomic DNA=(copies calculated from CART-BCMA standard curve)×correction factor/(amount DNA evaluated in nanograms)×1000 ng.

Measurement of Serum Cytokines:

Human cytokine magnetic 30-plex panel (LHC6003M) was from Life Technologies. Serum samples collected at baseline and at scheduled time-points out until 28 days post-infusion were cryopreserved at −80° C. Batched samples were thawed and analyzed according to the manufacturers' protocols. Assay plates were measured using a FlexMAP 3D instrument, and data acquisition and analysis were done using xPONENT software. Data quality was examined based on the following criteria. The standard curve for each analyte has a 5P R² value >0.95 with or without minor fitting using xPONENT software. To pass quality control, the results for an in-house control serum needed to be within the 95% of CI (confidence interval) derived from historical in-house control data for >25 of the tested analytes. No further tests were done on samples with results out of range low (<OOR). Samples with results that were out of range high (>OOR) or greater than two times the standard curve maximum value (SC max) were re-tested at higher dilutions. Results that passed the above quality controls or retests were reported.

Measurement of soluble BCMA, BAFF, and APRIL:

Antibody sets for human BCMA (DY193), APRIL (DY884B) and BAFF (DT124-05) were from R&D Systems. ELISA bead-strip and 4-column reservoir (SOW-A16735) were from Assay Depot. ELISA substrate ADHP (Ser. No. 10/010,469) was from Cayman Chemical. Assay plates (OX1263) were from E&K Scientific. All ELISA reagents were prepared according to the protocols for DuoSet ELISA except for Color Reagent B, which was supplemented with ADHP at 100 uM. Due to limited volumes of sera and lack of availability of a Luminex assay for BCMA, APRIL and BAFF, the three analytes were measured using ELISA bead-strips. Instead of coating the capture antibody (cAB) to the wells of an ELISA plate, it was coated on the surfaces of macrospheres, which enabled the measurement of all three analytes using 100 ul of serum. Assays were set up using bead-strips in assay plates based on an assay map following the protocol for antibody sets. At the end of the assay, one substrate plate per 12 bead-strips was prepared by adding 100 ul/well of substrate solution (1:1 of color reagent A and ADHP). Each bead-strip was placed in one column of the substrate plate according to the assay map. Color development was for 10 to 30 minutes. Plates were read on a FLUO STAR OMEGA instrument. Data quality control was performed as described for Luminex data.

Assessment of Bone Marrow Myeloma Cells, Including BCMA Expression:

Flow cytometry assessment of bone marrow aspirate material was performed directly on aspirate following a brief ammonium chloride red blood cell lysis step. The procedure was adapted from the EuroFlow protocol as described in Flores-Montero J, et al., Leukemia 2017; 31(10): 2094-103. Briefly, up to 2m1s of bone marrow aspirate was diluted with 48 mls of Pharm Lyse solution (BD Biosciences Cat #555899) and incubated for 15 minutes at room temperature on a shaking device. The cells were then collected by centrifugation for 10 minutes at 800 g, washed twice with flow cytometry buffer (PBS with 1% fetal bovine serum), stained with L/D Aqua viability dye (Thermo Fisher Cat #L34957). Surface staining was done with a mixture of antibodies to CD45, CD19, CD138, CD38, CD14, CD56, CD20, CD3, CD269 (BCMA), CD274 (PD-L1). FMO (fluorescence minus one) secondary only controls were used for BCMA evaluation. Aliquots of normal donor PBMC cells were stained in parallel as controls. The cells were then washed before permeabilization/fixation using Cytofix/Cytoperm reagent (BD Biosciences) for 20 minutes at room temperature, washed, and stained with a mixture of antibodies to kappa and lambda immunoglobulin light chains. The samples were then washed before resuspension in PBS and acquisition on a 17-color LSR Fortessa Special Order Research Product flow cytometer (BD) equipped with a violet, blue, green, and red laser. List mode files were analyzed using either FlowJo (Treestar) or FCS Express.

TABLE 42
Individual subject characteristics

| | | | | | | | | | | |
|---|---|---|---|---|---|---|---|---|---|---|
| | | | | | | | | | # | Last tx before |
| | | | | Yrs | | | | | lines | pheresis, |
| Sub. | Age | Sex | Race | from Dx | Isotype | Karyotype | FISH | Mutation | * | infusion** |

Cohort 1

| | | | | | | | | | | |
|---|---|---|---|---|---|---|---|---|---|---|
| 01 | 66 | M | white | 9.6 | IgGK | 46XY | +11, | NRAS, | 11 | Pom/Dex |
| | | | | | | | −16q, | TP53, | | Pom/Dex |
| | | | | | | | −17p | TP53 | | |
| 02 | 68 | M | white | 6.3 | IgAK | 46XY | +1q, | n/a | 9 | Carfilz/Len/Dex |
| | | | | | | | +4p, | | | Carfilz/Cyclo/Dex |
| | | | | | | | −17p | | | |
| 03 | 54 | F | white | 3.1 | IgAL | n/a | +1q, | n/a | 4 | D-PACE |
| | | | | | | | t(4; 14), | | | D-AC |
| | | | | | | | −16q | | | |
| 07 | 44 | M | white | 1.8 | K | n/a | +1q, −4, | NRAS, | 9 | Carfilz/Pano |
| | | | | | | | +11, | TP53 | | Pom/Dex-ACE |
| | | | | | | | −14, −16 | | | |
| 08 | 70 | M | white | 3.3 | IgAL | complex | +1q, | n/a | 6 | Carfilz/Pom/Dex |
| | | | | | | | −1p, −4, | | | Carfilz/Pom/Dex |
| | | | | | | | −17p | | | |
| 09 | 61 | M | white | 6.6 | K | 46XY | t(11; 14), | none | 9 | Daratumumab |
| | | | | | | | −16q, | | | Pulse Dex |
| | | | | | | | −17p | | | |
| 10 | 47 | M | white | 4.9 | IgGL | n/a | +1q, | KRAS, | 7 | Pom/Dex |
| | | | | | | | t(11; 14) | SF3B1 | | Pom/Cyclo/Dex + |
| | | | | | | | | | | plasma exchange |
| 11 | 57 | F | AA | 6.3 | IgGL | 46XX | +1q, | n/a | 10 | VDT-PACE |
| | | | | | | | t(4; 14), | | | VDT-PACE |
| | | | | | | | −17p | | | |
| 15 | 51 | F | Asian | 4.7 | IgAK | 46XX | +1q, | NRAS, | 7 | Pembro/Pom/Dex |
| | | | | | | | t(11; 14) | KRAS, | | Pom/Dex |
| | | | | | | | | BIRC3, | | |
| | | | | | | | | KIT | | |

Cohort 2

| | | | | | | | | | | |
|---|---|---|---|---|---|---|---|---|---|---|
| 12 | 51 | F | white | 2.8 | IgAL | 46XX | +1q, | TET2 | 5 | CyBorD |
| | | | | | | | −1p, | | | CyBorD |
| | | | | | | | t(4; 14), | | | |
| | | | | | | | +17p | | | |
| 13 | 59 | M | white | 9.4 | IgGL | 46XY | +11, | n/a | 10 | Salvage ASCT |
| | | | | | | | −16q, | | | VD-CE |
| | | | | | | | −17p | | | |
| 14 | 54 | F | white | 4.9 | IgGK | 46XX | t(11; 14), | n/a | 7 | Venetoclax |
| | | | | | | | −16q, | | | none |
| | | | | | | | −17p | | | |
| 16 | 54 | M | white | 4.1 | IgGK | complex | +1q, | BRAF | 7 | D-CE |
| | | | | | | | −16q, | | | D-CE + radiation |
| | | | | | | | −17p | | | |
| 22 | 45 | M | white | 2.7 | IgGL | 46XY | t(11; 14), | n/a | 7 | D-ACE |
| | | | | | | | +1q, | | | none |
| | | | | | | | −17p | | | |

Cohort 3

| | | | | | | | | | | |
|---|---|---|---|---|---|---|---|---|---|---|
| 17 | 64 | F | white | 5.0 | IgAK | 46XX | t(14; 16), | n/a | 7 | CPI-610 |
| | | | | | | | +1q, | | | D-AC |
| | | | | | | | −1p, | | | |
| | | | | | | | +11 | | | |
| 19 | 58 | M | white | 4.1 | L | 46XY | r(11; 14), | n/a | 5 | Dara/Ixa/Pom/Dex |
| | | | | | | | +1q, | | | Dara/Ixa/Pom/Dex |
| | | | | | | | +17, | | | |
| | | | | | | | −17p | | | |
| 20 | 61 | M | white | 2.6 | IgGL | n/a | t(4; 14), | n/a | 6 | Carfilz/cyclo/pom/ |
| | | | | | | | +1q, | | | dex |
| | | | | | | | −17p | | | VD-AC |
| 21 | 73 | M | white | 2.5 | IgAK | 46XY | +1q, | TET2 | 5 | Dara/Pom/Dex |
| | | | | | | | +14, | | | Bort/Pom/Dex |
| | | | | | | | +17, | | | |
| | | | | | | | −17p | | | |
| 23 | 63 | M | white | 2.3 | IgGK | 46XY | none | none | 6 | Carfilz/Pom/Dex/ |
| | | | | | | | | | | Nelfin |
| | | | | | | | | | | VD-PCE |
| 25 | 51 | M | AA | 4.5 | IgGK | 46XY | t(11; 14), | FLT3 | 7 | Selinexor/Dex |
| | | | | | | | +1q | | | Bort/Venetoclax/ |
| | | | | | | | | | | Dex + XRT |
| 27 | 58 | F | AA | 10.3 | IgGK | complex | t(11; 14), | BRAF, | 13 | Carfilz/Venetoclax/ |
| | | | | | | | +1q, | TET2 | | Dex |
| | | | | | | | −1p, −16, | | | Carfilz/Venetoclax/ |
| | | | | | | | −17p | | | Dex |
| 29 | 60 | F | white | 1.8 | IgGK | 45X,-X | none | TET2, | 3 | VDT-PACE |
| | | | | | | | | RBI, | | none |
| | | | | | | | | TP53 | | |
| 32 | 65 | F | white | 14.5 | IgGL | 46XX | t(4; 14), | TET2, | 6 | Carfilz/Cyclo/Dex |
| | | | | | | | +1q, | TET2 | | Carfilz/Cyclo/Dex |
| | | | | | | | −17 | | | |
| 33 | 75 | M | white | 8.8 | IgGK | hyperdip | +5, +9, | RAD21 | 5 | Pembro/Len/Dex |
| | | | | | | | +11, | | | none |
| | | | | | | | +15, | | | |
| | | | | | | | −17p | | | |
| 34 | 47 | M | white | 2.0 | IgGL | 46XY | +1q, | MYCN, | 4 | Carfilz/Pom/Dex |
| | | | | | | | −1p, | TPMT | | VD-PACE |
| | | | | | | | +14q | | | |

*A line of therapy was defined as per IMWG criteria. Radiation was not counted as a line.
**Most recent therapy received before T cell collection (“pheresis” - top line) and CART-BCMA infusion (“infusion” - bottom line). All therapy was held for at least 2 weeks prior to pheresis and again for at least 2 weeks prior to infusion (4 weeks for monoclonal antibodies). AA = African-American; ASCT = autologous stem cell transplant; Bort = bortezomib; Carfilz = carfilzomib; cyclo = cyclophosphamide; CPI-610 = investigational BET inhibitor; CyBorD = cyclophosphamide, bortezomib, dexamethasone; D-AC = dexamethasone + infusional doxorubicin and cyclophosphamide; D-ACE = dexamethasone + infusional doxorubicin, cyclophosphamide, and etoposide; D-CE: dexamethasone + infusional cyclophosphamide and etoposide; D-PACE = dexamethasone + infusional cisplatinum, doxorubicin, cyclophosphamide, and etoposide; Dara = daratumumab; Dex = dexamethasone; Dx = diagnosis; hyperdip = hyperdiploid; ixa = ixazomib; K = kappa light chain; L = lambda light chain; Len = lenalidomide; Nelfin = nelfinavir; Pano = panobinostat; Pembro = pembrolizumab; Pom = pomalidomide; Pom/Dex-ACE = pomalidomide, dexamethasone + infusional doxorubicin, cyclophosphamide, and etoposide; Tx = treatment; VD-AC = bortezomib, dexamethasone + infusional doxorubicin and cyclophosphamide; VD-CE = bortezomib, dexamethasone + infusional cyclophosphamide and etoposide; VD-PCE = bortezomib, dexamethasone + infusional cisplatinum, cyclophosphamide, etoposide; VDT- PACE = bortezomib, dexamethasone, thalidomide + infusional cisplatinum, doxorubicin, cyclophosphamide, and etoposide; Yrs = years.

TABLE 43
CART-BCMA manufacturing and product details

| | | | | | | | | | |
|---|---|---|---|---|---|---|---|---|---|
| | | CD4: | | | | | | | |
| | % CD3+ | CD8 in | | CD4: | | | Trans | Total | |
| | in seed | seed | % CD3+ | CD8 at | Fold | Pop | Eff | #CAR+ | Best |
| Sub. | culture | culture | at harvest | harvest | exp | dblgs | (%) | given (×10⁸) | response |

Cohort 1

| | | | | | | | | | |
|---|---|---|---|---|---|---|---|---|---|
| 01 | 52.6 | 2.04 | 98.4 | 2.40 | 43.5 | 4.54 | 9.6 | 2.0* | sCR |
| 02 | 63 | 0.40 | 69.1 | 1.31 | 12.7 | 3.67 | 18.2 | 5.0 | MR |
| 03 | 58.4 | 2.47 | 97.6 | 1.72 | 17.9 | 4.16 | 25.9 | 2.0* | VGPR |
| 07 | 20.3 | 1.47 | 94.9 | 1.60 | 38.9 | 5.28 | 24.2 | 5.0 | PR |
| 08 | 20.8 | 0.31 | 92.5 | 2.99 | 7.9 | 2.98 | 22.7 | 3.5 | PD |
| 09 | 86.8 | 0.49 | 87.5 | 1.69 | 10.0 | 3.33 | 22.2 | 5.0 | SD |
| 10 | 47.3 | 1.07 | 96.7 | 1.32 | 11.5 | 3.52 | 16.5 | 1.77 | PD |
| 11 | 21.4 | 0.81 | 96.9 | 1.20 | 8.7 | 3.11 | 19.4 | 5.0 | MR |
| 15 | 90.8 | 0.71 | 96 | 1.24 | 22.6 | 4.50 | 33.3 | 2.0* | VGPR |

Cohort 2

| | | | | | | | | | |
|---|---|---|---|---|---|---|---|---|---|
| 12 | 80 | 0.38 | 96.2 | 3.23 | 31.7 | 4.98 | 21.7 | 0.5 | SD |
| 13 | 72 | 0.50 | 86.9 | 1.72 | 14.0 | 3.84 | 24.2 | 0.5 | MR |
| 14 | 94.6 | 0.81 | 98.9 | 2.21 | 60.4 | 5.92 | 31.8 | 0.5 | SD |
| 16 | 50.9 | 1.06 | 98.1 | 0.84 | 30.6 | 4.94 | 21.9 | 0.5 | PR |
| 22 | 65.1 | 0.46 | 98.4 | 1.39 | 49.3 | 5.62 | 11 | 0.5 | SD |

Cohort 3

| | | | | | | | | | |
|---|---|---|---|---|---|---|---|---|---|
| 17 | 84.4 | 0.59 | 97.3 | 1.87 | 56.8 | 5.83 | 27.4 | 5.0 | PR |
| 19 | 88.1 | 0.91 | 94.9 | 2.09 | 16.8 | 4.07 | 13.7 | 5.0 | CR |
| 20 | 70 | 0.70 | 97.4 | 2.17 | 32.3 | 5.02 | 8.4 | 5.0 | VGPR |
| 21 | 83.5 | 0.46 | 98.9 | 3.26 | 45.9 | 5.52 | 7.5 | 5.0 | SD |
| 23 | 74.7 | 0.42 | 97.6 | 1.46 | 45.1 | 5.77 | 11 | 5.0 | MR |
| 25 | 69.2 | 0.54 | 99 | 0.93 | 23.7 | 4.57 | 10.7 | 2.0* | PR |
| 27 | 69 | 0.91 | 97.9 | 0.90 | 25.1 | 4.65 | 15 | 5.0 | VGPR |
| 29 | 29.8 | 2.49 | 91.7 | 4.55 | 23.0 | 4.52 | 8.3 | 5.0 | SD |
| 32 | 71 | 1.23 | 98.3 | 1.87 | 40.0 | 5.32 | 8.9 | 5.0 | MR |
| 33 | 83.6 | 0.33 | 96.5 | 1.78 | 31.1 | 4.96 | 8.6 | 5.0 | PR |
| 34 | 62.7 | 1.73 | 96.4 | 4.02 | 29.3 | 4.87 | 14.7 | 5.0 | VGPR |

Frequency of total CD3+, CD3+CD4+, and CD3+CD8+ cells was assessed by flow cytometry at the start of manufacturing (“seed culture”, after elutriation), and at the end of manufacturing (“at harvest”). Aph = apheresis product; fold exp = fold expansion; pop dblgs = population doublings; trans eff = transduction efficiency. MR = minimal response; PD = progressive disease; PR = partial response; sCR = stringent complete response; SD = stable disease; VGPR = very good partial response
*Subjects 01, 03, 15, and 25 received only 40% of planned dose due to fevers/early CRS.

TABLE 44
Individual subject adverse events

| | | | | | |
|---|---|---|---|---|---|
| Sub. | Grade 1 | Grade 2 | Grade 3 | Grade 4 | Grade 5 |

Cohort 1

| | | | | | |
|---|---|---|---|---|---|
| 01 | Dry eye | DIC | Anemia | Lymphopenia | |
| | Dry mouth | Vertigo | Fatigue | | |
| | Vomiting | Ileus | CRS | | |
| | Flu-like symptoms | Nausea | Alk phos increased | | |
| | Metabolism disorder - | Fever | AST increased | | |
| | other (iron | Lung infection | Hypocalcemia | | |
| | deficiency) | Creatinine increased | Hypokalemia | | |
| | Myalgia | Thrombocytopenia | Hypophosphatemia | | |
| | Headache | Musculoskeletal | | | |
| | Ischemia | disorder - other | | | |
| | cerebrovascular | (generalized muscle | | | |
| | Nervous system | aches) | | | |
| | disorder - other | Urinary retention | | | |
| | (confusion) | Respiratory disorder - | | | |
| | Vascular disorder - | other (pulmonary | | | |
| | other (edema) | edema and atelectasis) | | | |
| | | Skin/SQ disorder - | | | |
| | | other (skin tear) | | | |
| | | Hypertension | | | |
| 02 | Sinus tachycardia | | Thrombocytopenia | | |
| | Gastrointestinal | | | | |
| | disorders - other | | | | |
| | (loose stool) | | | | |
| | Nausea | | | | |
| | Fatigue | | | | |
| | CRS | | | | |
| | Dizziness | | | | |
| | Headache | | | | |
| | Respiratory disorder - | | | | |
| | other (nasal and | | | | |
| | sinus congestion, | | | | |
| | cough) | | | | |
| 03 | Sinus bradycardia | SVT | CRS | Neutropenia | |
| | Ear disorder - other | Fatigue | UTI | Leukopenia | |
| | (ears feel full, hard to | Infection - other (S. | Fibrinogen | Hypocalcemia | |
| | hear) | mitis/S. oralis | decreased | RPLS | |
| | Eye disorder - other | bacteremia) | Hypophosphatemia | Delirium | |
| | (difficulty reading) | Upper respiratory | Seizure | | |
| | Constipation | infection | Pleural effusion | | |
| | Edema | Lymphopenia | Hypertension | | |
| | Fever | Hypernatremia | | | |
| | Hypokalemia | Musculoskeletal | | | |
| | Hypomagnesemia | disorder - other | | | |
| | Bone pain | (deconditioning, pain | | | |
| | Concentration | base of skull) | | | |
| | impairment | Intracranial | | | |
| | Headache | hemorrhage | | | |
| | Hoarseness | Respiratory disorder - | | | |
| | Skin/SQ disorder - | other (post-nasal drip) | | | |
| | other (ecchymosis) | Hypotension | | | |
| 07 | Infection - other | CRS | Anemia | | |
| | (upper respiratory | Dyspepsia | Hypokalemia | | |
| | infection) | Hypoalbuminemia | Hyponatremia | | |
| | Skin/SQ disorder - | Hypocalcemia | | | |
| | other (rash on face) | Respiratory, thoracic | | | |
| | | disorders - other | | | |
| | | (hemothorax) | | | |
| 08 | Sinus tachycardia | | Lung infection | CRS | Death |
| | General disorder - | | Delirium | Thrombocytopenia | NOS |
| | other (rigors) | | | Encephalopathy | |
| | | | | Infection - other | |
| | | | | (fungemia) | |
| 09 | Constipation | Anemia | Lung infection | | |
| | Chills | Pericardial effusion | | | |
| | Fatigue | CRS | | | |
| | Thrombocytopenia | Neutropenia | | | |
| | Anorexia | Hypocalcemia | | | |
| | Hypokalemia | Headache | | | |
| 10 | Chills | Thromobocytopenia | | | |
| | Fever | | | | |
| 11 | Nausea | CRS | Hypophosphatemia | | |
| | Fatigue | Infection - other | | | |
| | Thrombocytopenia | (Clostridium difficile) | | | |
| | Headache | Hypocalcemia | | | |
| | Respiratory disorder - | | | | |
| | other (cough, | | | | |
| | congestion) | | | | |
| 15 | Hypocalcemia | Fatigue | Thrombocytopenia | Neutropenia | |
| | Hypokalemia | CRS | Hypoglycemia | | |
| | Headache | Infection - other | | | |
| | Cough | (positive test for Shiga | | | |
| | Dyspnea | toxin) | | | |
| | Respiratory disorders - | Alk phos increased | | | |
| | other (nasal | Hypotension | | | |
| | congestion) | | | | |
| | Alopecia | | | | |

Cohort 2

| | | | | |
|---|---|---|---|---|
| 12 | Anemia | Infection - other | Febrile neutropenia | Sepsis |
| | Vomiting | (adenovirus) | | Neutropenia |
| | Fatigue | | | Leukopenia |
| | Fever | | | |
| | Hypocalcemia | | | |
| 13 | Diarrhea | Upper respiratory | Neutropenia | |
| | Fatigue | infection | | |
| | ALT increased | | | |
| | AST increased | | | |
| | Anorexia | | | |
| | Dizziness | | | |
| | Pruritis | | | |
| | Skin/subcutaneous | | | |
| | tissue disorder - other | | | |
| | (lesions on bilateral | | | |
| | ears) | | | |
| 14 | Back pain | CRS | Anemia | Neutropenia |
| | Pain in extremity | | Febrile neutropenia | Leukopenia |
| 16 | Colitis | Flu-like symptoms | Febrile neutropenia | Lymphopenia |
| | Hypocalcemia | CRS | | Neutropenia |
| | Hypokalemia | Hypoalbuminemia | | Thrombocytopenia |
| | Hypomagnesemia | Epistaxis | | Leukopenia |
| | Paresthesia | | | |
| | Confusion | | | |
| | Hoarseness | | | |
| | Sore throat | | | |
| | Hypotension | | | |
| 22 | Fatigue | Nausea | Febrile neutropenia | Lymphopenia |
| | Myalgia | CRS | | Leukopenia |
| | Insomnia | | | |

Cohort 3

| | | | | |
|---|---|---|---|---|
| 17 | Diarrhea | Chills | | Neutropenia |
| | Hypocalcemia | CRS | | Leukopenia |
| | Hypokalemia | Arthralgia | | |
| | Back pain | | | |
| | Headache | | | |
| | Peripheral sensory | | | |
| | neuropathy | | | |
| | Insomnia | | | |
| 19 | Nausea | Ear and labyrinth | CRS | |
| | Chills | disorders - other | Lymphopenia | |
| | | (seasonal allergies) | Hypophosphatemia | |
| | | Gastrointestinal | Hypertension | |
| | | disorders - other | | |
| | | (food poisoning) | | |
| | | Fever | | |
| | | Sinusitis | | |
| | | Upper respiratory | | |
| | | infection | | |
| 20 | Constipation | Fatigue | CRS | AST increased |
| | Diarrhea | ALT increased | Hyponatremia | |
| | Vomiting | Hypophosphatemia | | |
| | Chills | Dysphasia | | |
| | CPK increased | Lethargy | | |
| | Hypokalemia | Confusion | | |
| | Back pain | Hypotension | | |
| | Allergic rhinitis | | | |
| 21 | Alk phos increased | CRS | Lymphopenia | |
| | Hypocalcemia | Skin disorders - other | Neutropenia | |
| | Hyponatremia | (cellulitis) | Thrombocytopenia | |
| | Epistaxis | | Leukopenia | |
| 23 | Anal hemorrhage | CRS | Febrile neutropenia | |
| | Nausea | Abdominal pain | | |
| | Edema limbs | Colitis | | |
| | Hypoalbuminemia | Diarrhea | | |
| | Hypocalcemia | Vomiting | | |
| | Tremor | Pain | | |
| | | Thrombocytopenia | | |
| | | Hypokalemia | | |
| 25 | Nausea | Hypoalbuminemia | CRS | |
| | Facial pain | Hypokalemia | Hypocalcemia | |
| | Fatigue | Hypercalcemia | Papilledema | |
| | Hypomagnesemia | | | |
| | Headache | | | |
| | Peripheral sensory | | | |
| | neuropathy | | | |
| 27 | Diarrhea | Sinus bradycardia | CRS | Anemia |
| | Nausea | Edema limbs | SVT | Thrombocytopenia |
| | Oral pain | Urinary tract infection | AST increased | Hyperglycemia |
| | Fatigue | Creatinine increased | Ejection fraction | Hypocalcemia |
| | Pain | Neutropenia | decreased | Hypokalemia |
| | Infections - other | Hypernatremia | Lymphopenia | Pleural |
| | (upper respiratory | Wheezing | Leukopenia | hemorrhage |
| | infection) | | Hypoalbuminemia | |
| | Back pain | | Hyponatremia | |
| | Myalgia | | Encephalopathy | |
| | Pain in extremity | | Hypertension | |
| | Dry skin | | | |
| | Hematoma | | | |
| 29 | Nausea | Chills | Abdominal pain | Lymphopenia |
| | Fatigue | CRS | Hyponatremia | Leukopenia |
| | Pain | Hypoalbuminemia | Hypophosphatemia | |
| | Respiratory disorders - | | | |
| | Other (scratchy | | | |
| | throat, slight cough) | | | |
| 32 | Nausea | Diarrhea | Anemia | Lymphopenia |
| | Fatigue | Encephalopathy | CRS | Thrombocytopenia |
| | Alopecia | Allergic rhinitis | Neutropenia | Leukopenia |
| | | | Hypoalbuminemia | |
| | | | Hypocalcemia | |
| | | | Hyponatremia | |
| | | | Hypophosphatemia | |
| 33 | Oral pain | Chills | Febrile neutropenia | Neutropenia |
| | Diarrhea | CRS | | |
| | Fatigue | Hypoalbuminemia | | |
| | Flu-like symptoms | | | |
| | Arthralgia | | | |
| | Flank pain | | | |
| | Allergic rhinitis | | | |
| 34 | Diarrhea | CRS | Leukopenia | Lymphopenia |
| | Nausea | Thrombocytopenia | Hypophosphatemia | Neutropenia |
| | Sinus tachycardia | Hypocalcemia | Hyopnatremia | |
| | Confusion | Flu-like symptoms | | |
| | ALT increased | AST increased | | |

All events regardless of attribution are listed. If an event occurred more than once in same patient, highest grade is reported.
Alk phos = alkaline phosphatase; ALT = alanine aminotransferase; AST = aspartate aminotransferase; CPK = creatine phosphokinase; CRS = cytokine release syndrome; DIC = disseminated intravascular coagulation; NOS = not otherwise specified; RPLS = reversible posterior leukoencephalopathy syndrome (also known as posterior reversible encephalopathy syndrome (PRES)); SQ = subcutaneous; SVT = supraventricular tachycardia; UTI = urinary tract infection

TABLE 45
Characteristics of peripheral blood CART-BCMA+ cells at peak expansion

| | | | | | | |
|---|---|---|---|---|---|---|
| | Day of | % CAR+ | % CAR+ | % CAR+ | % HLADR+ | Best |
| Sub. | peak | of CD3+ | of CD4+ | of CD8+ | of CAR+ | response |

Cohort 1

| | | | | | | |
|---|---|---|---|---|---|---|
| 01 | 10 | 89.5 | 8.6 | 92.3 | 97 | sCR |
| 02 | 9 | 2.8 | 1.4 | 5.0 | 82 | MR |
| 03 | 14 | 76.2 | 13.7 | 85.9 | 97 | VGPR |
| 07 | 10 | 29.2 | 7.3 | 39.3 | 89 | PR |
| 08 | 16 | 9.3 | 0.4 | 16.9 | 95 | PD |
| 09 | 11 * | * | * | * | * | SD |
| 10 | 30 | 1.9 | 0.1 | 2.0 | 21 | PD |
| 11 | 8 | 18.2 | 14.6 | 25.2 | 94 | MR |
| 15 | 10 | 28.1 | 6.3 | 51.0 | 96 | VGPR |

Cohort 2

| | | | | | | |
|---|---|---|---|---|---|---|
| 12 | 10 | 2.0 | 0.8 | 2.3 | 76 | SD |
| 13 | 13 | 8.4 | 2.9 | 13.2 | 92 | MR |
| 14 | 10 | 7.8 | 3.5 | 10.6 | 95 | SD |
| 16 | 14 | 47.1 | 12.1 | 50.3 | 98 | PR |
| 22 | 9 | 4.3 | 0.6 | 8.6 | 94 | SD |

Cohort 3

| | | | | | | |
|---|---|---|---|---|---|---|
| 17 | 9 | 22.1 | 10.4 | 27.4 | 96 | PR |
| 19 | 15 | 9.7 | 8.9 | 10.2 | 87 | CR |
| 20 | 15 | 59.2 | 22.4 | 65.1 | 99 | VGPR |
| 21 | 9 | 16.0 | 9.2 | 19.9 | 97 | SD |
| 23 | 10 | 13.8 | 6.6 | 17.3 | 93 | MR |
| 25 | 10 | 5.1 | 3.8 | 5.9 | 85 | PR |
| 27 | 14 | 42.9 | 4.9 | 47.3 | 88 | VGPR |
| 29 | 7 | 4.1 | 2.8 | 15.0 | 77 | SD |
| 32 | 10 | 36.2 | 6.1 | 46.4 | 97 | MR |
| 33 | 11 | 41.8 | 29.8 | 46.9 | 91 | PR |
| 34 | ** | ** | ** | ** | ** | VGPR |

CART-BCMA cells were assessed by flow cytometry as in FIG. 38. At day of peak expansion, the frequency of CAR+ cells within CD3+, CD4+, and CD8+ populations are listed. Activation status at peak expansion (as measured by % of CAR+ cells expressing HLA-DR) is also shown. MR = minimal response; PD = progressive disease; PR = partial response; sCR = stringent complete response; SD = stable disease; VGPR = very good partial response
* Peak determined by qPCR; CAR+ cells not detectable by flow.
** No sample available between days 10-21 so peak could not be determined.

TABLE 46
CART-BCMA engraftment by qPCR in blood, bone marrow, and other sites

| | | | | | |
|---|---|---|---|---|---|
| | Day 28 | Day 28 | 3 month | 3 month | |
| Sub. | blood | BM | blood | BM | Other (site, day) |

Cohort 1

| | | | | | |
|---|---|---|---|---|---|
| 01 | 23447.33 | 10458.66 | 4373.95 | n/a | n/a |
| 02 | 454.57 | 383.62 | 334.90 | n/a | n/a |
| 03 | 220081.08* | 109964.97* | 9517.67 | 9934.97 | CSF, D4: 3285.88 |
| | | | | | CSF, D16: 158038.74 |
| | | | | | Pleural fluid, D8: 23941.97 |
| | | | | | Pleural fluid, D18: 144516.62 |
| 07 | 1757.60 | 2140.31 | n/a | n/a | n/a |
| 08 | n/a | n/a | n/a | n/a | n/a |
| 09 | 32.15 | 16.75 | n/a | n/a | n/a |
| 10 | 62.64 | 0.00 | n/a | n/a | n/a |
| 11 | 731.44 | n/a | 218.26 | n/a | n/a |
| 15 | 724.49 | 1084.16 | 32.58 | 99.48 | n/a |

Cohort 2

| | | | | | |
|---|---|---|---|---|---|
| 12 | 140.60 | 25.59 | n/a | n/a | n/a |
| 13 | 193.80 | 43.64 | 94.70 | 27.04 | n/a |
| 14 | 66.69 | 255.76 | 8.80 | 0.00 | n/a |
| 16 | 25919.10 | 22077.89 | n/a | n/a | n/a |
| 22 | 34.56 | 22.09 | 77.71 | 63.76 | n/a |

Cohort 3

| | | | | | |
|---|---|---|---|---|---|
| 17 | 1639.98 | 1398.82 | 1392.25 | 649.69 | n/a |
| 19 | 12987.83 | 6578.03 | 153.88 | 117.53 | n/a |
| 20 | 43463.65 | 28086.46 | 4107.95 | 5223.76 | n/a |
| 21 | 8372.96 | 1161.92 | 96.11 | n/a | n/a |
| 23 | 5946.81 | n/a | 102.68 | n/a | n/a |
| 25 | 256.33 | 259.97 | 149.20 | 160.33 | n/a |
| 27 | 51014.95 | 41242.37 | 14271.48 | 9634.66 | Pleural fluid, D15: 58511.99 |
| 29 | 738.84 | 210.05 | n/a | n/a | n/a |
| 32 | 6983.98 | 7055.94 | 58.15 | 15.72 | n/a |
| 33 | 7539.99 | 5142.13 | 230.66 | 39.09 | n/a |
| 34 | 5199.07 | 1512.03 | n/a | n/a | n/a |

CART-BCMA levels (copies/μg genomic DNA) were generally comparable in blood and marrow at tested timepoints. CART-BCMA was found at high levels in CSF and pleural fluid of subject 03, and pleural fluid of subject 27.
BM = bone marrow; CSF = cerebrospinal fluid. n/a = not available.
*Assays performed on day 45.

TABLE 47
Peak fold increase in serum cytokines and
severity of cytokine release syndrome (CRS)

| | | Median peak | |
|---|---|---|---|
| | Median peak | fold increase: | |
| | fold increase: | CRS Gr 3-4 or | |
| Cytokine | CRS Gr 0-2 | Gr 2 + toci | p-value |
| IL-6 | 3.88 | 76.26 | <0.0001 |
| IFN-γ | 1.26 | 11.95 | <0.0001 |
| IL-2Rα | 4.13 | 18.40 | <0.0001 |
| MIP-1α | 1.35 | 3.47 | <0.0001 |
| IL-15 | 1.71 | 12.81 | 0.0004 |
| IL-1RA | 2.33 | 19.68 | 0.0036 |
| MCP-1 | 2.11 | 6.99 | 0.0044 |
| GM-CSF | 2.69 | 36.27 | 0.0052 |
| IL-1β | 1.28 | 2.23 | 0.0052 |
| MIG | 6.81 | 39.14 | 0.008 |
| MIP-1β | 1.49 | 2.39 | 0.013 |
| IL-5 | 2.84 | 35.32 | 0.019 |
| VEGF | 1.93 | 3.68 | 0.022 |
| IL-17 | 1.16 | 3.13 | 0.026 |
| IL-7 | 2.24 | 6.18 | 0.10 |
| IP-10 | 6.63 | 10.97 | 0.10 |
| IL-8 | 8.65 | 30.72 | 0.20 |
| IL-4 | 1.29 | 1.95 | 0.24 |
| IL-10 | 13.84 | 15.82 | 0.53 |

Serum cytokine concentrations in μg/ml through day 28 were measured by Luminex assay. The median peak fold increase over baseline for each cytokine listed for subjects with no CRS, grade 1 CRS, or grade 2 CRS not receiving tocilizumab (CRS gr 0-2) was compared to median peak fold increase for subjects with grade 3-4 CRS or grade 2 CRS receiving tocilizumab (CRS Gr 3-4 or Gr 2 + toci). Exact p-value by Mann-Whitney test is listed when applicable.

TABLE 48
Peak fold increase in serum cytokines and neurotoxicity

| | Median peak | Median peak | |
|---|---|---|---|
| | fold increase: | fold increase: | |
| Cytokine | No neurotox | Any neurotox | p-value |
| IL-6 | 3.92 | 76.26 | 0.0002 |
| IFN-γ | 1.28 | 15.77 | 0.0002 |
| IL-1RA | 2.33 | 19.68 | 0.0015 |
| MIP-1α | 1.53 | 3.47 | 0.006 |
| GM-CSF | 3.46 | 36.27 | 0.013 |
| IL-15 | 1.76 | 10.54 | 0.016 |
| IL-1β | 1.32 | 3.70 | 0.018 |
| IP-10 | 5.80 | 20.44 | 0.019 |
| IL-7 | 3.29 | 10.28 | 0.019 |
| IL-2Rα | 4.46 | 20.86 | 0.019 |
| VEGF | 1.93 | 3.68 | 0.050 |
| MIP-1β | 1.56 | 2.24 | 0.056 |
| MIG | 7.17 | 39.14 | 0.057 |
| MCP-1 | 2.13 | 6.41 | 0.066 |
| IL-8 | 8.65 | 36.77 | 0.12 |
| IL-17 | 1.32 | 1.57 | 0.19 |
| IL-5 | 5.15 | 8.54 | 0.26 |
| IL-4 | 1.47 | 2.20 | 0.40 |
| IL-10 | 13.70 | 27.51 | 0.41 |

Serum cytokine concentrations in μg/ml through day 28 were measured by Luminex assay.
The median peak fold increase over baseline for each cytokine listed for subjects with no neurotoxicity (neurotox) was compared to median peak fold increase for subjects with any grade neurotoxicity. Exact p-value by Mann-Whitney test is listed.

TABLE 49
Details of BCMA expression on myeloma cells

| | | | | | | | | | | |
|---|---|---|---|---|---|---|---|---|---|---|
| | | Pre | Pre | | D 28 | D 28 | | D 90 | D 90 | |
| | Pre | FMO | BCMA | D 28 | FMO | BCMA | D 90 | BCMA | BCMA | Best |
| Sub | %+ | MFI | MFI | %+ | MFI | MFI | % + | MFI | MFI | response |

Cohort 1

| | | | | | | | | | | |
|---|---|---|---|---|---|---|---|---|---|---|
| 01 | 95 | 568 | 10864 | n/a | n/a | n/a | n/a | n/a | n/a | sCR |
| 02 | 93 | 355 | 2375 | 97 | 483 | 3254 | n/a | n/a | n/a | MR |
| 03 | 84 | 265 | 1873 | n/a | n/a | n/a | 19* | 78* | 268* | VGPR |
| 07 | n/a | n/a | n/a | 84 | 273 | 1426 | n/a | n/a | n/a | PR |
| 08 | 99 | 124 | 6859 | n/a | n/a | n/a | n/a | n/a | n/a | PD |
| 09 | 28 | 53 | 206 | 94 | 204 | 1116 | n/a | n/a | n/a | SD |
| 10 | 83 | 192 | 1998 | 99 | 54 | 2140 | n/a | n/a | n/a | PD |
| 11 | 81 | 332 | 1844 | 62 | 49 | 832 | n/a | n/a | n/a | MR |
| 15 | 99 | 249 | 24842 | n/a | n/a | n/a | 100 | 362 | 30685 | VGPR |

Cohort 2

| | | | | | | | | | | |
|---|---|---|---|---|---|---|---|---|---|---|
| 12 | 100 | −142 | 6755 | 100 | 187 | 7268 | n/a | n/a | n/a | SD |
| 13 | n/a | n/a | n/a | n/a | n/a | n/a | n/a | n/a | n/a | MR |
| 14 | 99 | 192 | 4317 | 98 | 172 | 2388 | n/a | n/a | n/a | SD |
| 16 | 97 | 366 | 4039 | 48 | 394 | 850 | 93 | 488 | 3819 | PR |
| 22 | 100 | 321 | 5947 | 99 | 271 | 4947 | 100 | 126 | 5599 | SD |

Cohort 3

| | | | | | | | | | | |
|---|---|---|---|---|---|---|---|---|---|---|
| 17 | 87 | 646 | 2667 | 27 | 295 | 784 | 76 | 350 | 1543 | PR |
| 19 | 98 | 429 | 2923 | 65 | 316 | 992 | n/a | n/a | n/a | CR |
| 20 | 89 | 394 | 2643 | 29 | 369 | 944 | 36 | 185 | 377 | VGPR |
| 21 | 99 | 314 | 2704 | 86 | 252 | 1108 | n/a | n/a | n/a | SD |
| 23 | n/a | n/a | n/a | n/a | n/a | n/a | n/a | n/a | n/a | MR |
| 25 | 97 | 283 | 4000 | 54 | 259 | 833 | 92 | 223 | 2501 | PR |
| 27 | 100 | 618 | 6548 | 74 | 242 | 964 | 70 | 159 | 616 | VGPR |
| 29 | n/a | n/a | n/a | n/a | n/a | n/a | n/a | n/a | n/a | SD |
| 32 | 92 | 176 | 3482 | 85 | 169 | 1472 | n/a | n/a | n/a | MR |
| 33 | n/a | n/a | n/a | n/a | n/a | n/a | n/a | n/a | n/a | PR |
| 34 | 100 | 303 | 17109 | 92 | 1607 | 5001 | 100 | 309 | 11232 | VGPR |

Bone marrow myeloma cells were gated and BCMA expression analyzed as per FIG. 55. The percentage of myeloma cells expressing BCMA (% +), as well as the mean fluorescence intensity (MFI) for BCMA and FMO (fluorescence minus one) negative control are depicted, n/a = not available. Pre = pre-treatment. D 28 = day 28 post-treatment. D 90 = day 90 post-treatment. Sub = subject.
*actually D 164.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method of evaluating or predicting a subject's responsiveness to a BCMA CAR-expressing cell therapy, wherein the subject has a disease associated with the expression of BCMA, comprising:
acquiring a value for one, two, three, four, five, or all of:
(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,
(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(iv) the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)), or
(vi) the proliferation of seeded cells from the subject during manufacturing of the BCMA CAR-expressing cell therapy, e.g., as measured by population doublings by day 9 (PDL9), wherein:
(a) an increase in the value of one, two, three, four, five, or all of (i)-(vi), as compared to a reference value, e.g., a non-responder reference value, is indicative or predictive of increased responsiveness of the subject to the BCMA CAR-expressing cell therapy; or
(b) a decrease in the value of one, two, three, four, five, or all of (i)-(vi), as compared to a reference value, e.g., a responder reference value, is indicative or predictive of decreased responsiveness of the subject to the BCMA CAR-expressing cell therapy,
thereby evaluating or predicting the subject's responsiveness to the BCMA CAR-expressing cell therapy.

2. The method of claim 1, wherein the increase in the value of one, two, three, four, five, or all of (i)-(vi), as compared to a reference value, e.g., a non-responder reference value, is indicative or predictive of one, two, three, or all of:
(a) increased responsiveness of the subject to the BCMA CAR-expressing cell therapy;
(b) the subject as a responder of the BCMA CAR-expressing cell therapy;
(c) the subject as suitable for the BCMA CAR-expressing cell therapy; or
(d) increased expansion of the BCMA CAR-expressing cell therapy in the subject, e.g., as measured by an assay disclosed herein, e.g., as measured by the copy number of CAR transgenes per μg DNA using qPCR.

3. The method of claim 1 or 2, wherein the decrease in the value of one, two, three, four, five, or all of (i)-(vi), as compared to a reference value, e.g., a responder reference value, is indicative or predictive of one, two, or all of:
(a) decreased responsiveness of the subject to the BCMA CAR-expressing cell therapy;
(b) the subject as a non-responder of the BCMA CAR-expressing cell therapy; or
(c) decreased expansion of the BCMA CAR-expressing cell therapy in the subject, e.g., as measured by an assay disclosed herein, e.g., as measured by the copy number of CAR transgenes per μg DNA using qPCR.

4. The method of any one of claims 1-3, wherein the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry.

5. The method of claim 4, wherein the ratio being:
(1) greater than or equal to 1 (e.g., between 1 and 5, e.g., between 1 and 3.5); or
(2) greater than or equal to 1.6 (e.g., between 1.6 and 5, e.g., between 1.6 and 3.5),
is indicative or predictive of one, two, three, or all of:
(a) increased responsiveness of the subject to the BCMA CAR-expressing cell therapy;
(b) the subject as a responder of the BCMA CAR-expressing cell therapy;
(c) the subject as suitable for the BCMA CAR-expressing cell therapy; or
(d) increased expansion of the BCMA CAR-expressing cell therapy in the subject, e.g., as measured by an assay disclosed herein, e.g., as measured by the copy number of CAR transgenes per μg DNA using qPCR.

6. The method of claim 4 or 5, wherein the ratio being less than 1 (e.g., between 0.001 and 1) is indicative or predictive of one, two, or all of:
(a) decreased responsiveness of the subject to the BCMA CAR-expressing cell therapy;
(b) the subject as a non-responder of the BCMA CAR-expressing cell therapy; or
(c) decreased expansion of the BCMA CAR-expressing cell therapy in the subject, e.g., as measured by an assay disclosed herein, e.g., as measured by the copy number of CAR transgenes per μg DNA using qPCR.

7. The method of any one of claims 1-6, wherein the value for the level or activity of CD8+ Tscm (stem cell memory T cells) comprises the percentage of CD8+ Tscm (stem cell memory T cells) among CD8+ T cells, e.g., as measured by an assay disclosed herein, e.g., flow cytometry.

8. The method of any one of claims 1-7, wherein the value for the level or activity of HLADR-CD95+CD27+CD8+ cells comprises the percentage of HLADR-CD95+CD27+CD8+ cells among CD8+ T cells, e.g., as measured by an assay disclosed herein, e.g., flow cytometry.

9. The method of claim 8, wherein the percentage of HLADR-CD95+CD27+CD8+ cells among CD8+ T cells being greater than or equal to 25% (e.g., between 30% and 90%, e.g., between 35% and 85%, e.g., between 40% and 80%, e.g., between 45% and 75%, e.g., between 50% and 75%) is indicative or predictive of one, two, three, or all of:
(a) increased responsiveness of the subject to the BCMA CAR-expressing cell therapy;
(b) the subject as a responder of the BCMA CAR-expressing cell therapy;
(c) the subject as suitable for the BCMA CAR-expressing cell therapy; or
(d) increased expansion of the BCMA CAR-expressing cell therapy in the subject, e.g., as measured by an assay disclosed herein, e.g., as measured by the copy number of CAR transgenes per μg DNA using qPCR.

10. The method of claim 8 or 9, wherein the percentage of HLADR-CD95+CD27+CD8+ cells among CD8+ T cells being less than 25% (e.g., between 0.1% and 25%, e.g., between 0.1% and 22%, e.g., between 0.1% and 20%, e.g., between 0.1% and 18%, e.g., between 0.1% and 15%)) is indicative or predictive of one, two, or all of:
(a) decreased responsiveness of the subject to the BCMA CAR-expressing cell therapy;
(b) the subject as a non-responder of the BCMA CAR-expressing cell therapy; or
(c) decreased expansion of the BCMA CAR-expressing cell therapy in the subject, e.g., as measured by an assay disclosed herein, e.g., as measured by the copy number of CAR transgenes per μg DNA using qPCR.

11. The method of any one of claims 1-10, wherein the value for the level or activity of CD45RO− CD27+CD8+ cells comprises the percentage of CD45RO−CD27+CD8+ cells among CD8+ T cells, e.g., as measured by an assay disclosed herein, e.g., flow cytometry.

12. The method of claim 11, wherein the percentage of CD45RO−CD27+CD8+ cells among CD8+ T cells being greater than or equal to 20% (e.g., between 20% and 90%, e.g., between 20% and 80%, e.g., between 20% and 70%, e.g., between 20% and 60%) is indicative or predictive of one, two, three, or all of:
(a) increased responsiveness of the subject to the BCMA CAR-expressing cell therapy;
(b) the subject as a responder of the BCMA CAR-expressing cell therapy;
(c) the subject as suitable for the BCMA CAR-expressing cell therapy; or
(d) increased expansion of the BCMA CAR-expressing cell therapy in the subject, e.g., as measured by an assay disclosed herein, e.g., as measured by the copy number of CAR transgenes per μg DNA using qPCR.

13. The method of claim 11 or 12, wherein the percentage of CD45RO−CD27+CD8+ cells among CD8+ T cells being less than 20% (e.g., between 0.1% and 20%, e.g., between 0.1% and 18%, e.g., between 0.1% and 15%, e.g., between 0.1% and 12%, e.g., between 0.1% and 10%) is indicative or predictive of one, two, or all of:
(a) decreased responsiveness of the subject to the BCMA CAR-expressing cell therapy;
(b) the subject as a non-responder of the BCMA CAR-expressing cell therapy; or
(c) decreased expansion of the BCMA CAR-expressing cell therapy in the subject, e.g., as measured by an assay disclosed herein, e.g., as measured by the copy number of CAR transgenes per μg DNA using qPCR.

14. The method of any one of claims 1-13, wherein the value for the level or activity of CCR7+CD45RO−CD27+CD8+ cells comprises the percentage of CCR7+CD45RO−CD27+CD8+ cells among CD8+ T cells, e.g., as measured by an assay disclosed herein, e.g., flow cytometry.

15. The method of claim 14, wherein the percentage of CCR7+CD45RO−CD27+CD8+ cells among CD8+ T cells being greater than or equal to 15% (e.g., between 15% and 90%, e.g., between 15% and 80%, e.g., between 15% and 70%, e.g., between 15% and 60%, e.g., between 15% and 50%) is indicative or predictive of one, two, three, or all of:
(a) increased responsiveness of the subject to the BCMA CAR-expressing cell therapy;
(b) the subject as a responder of the BCMA CAR-expressing cell therapy;
(c) the subject as suitable for the BCMA CAR-expressing cell therapy; or
(d) increased expansion of the BCMA CAR-expressing cell therapy in the subject, e.g., as measured by an assay disclosed herein, e.g., as measured by the copy number of CAR transgenes per μg DNA using qPCR.

16. The method of claim 14 or 15, wherein the percentage of CCR7+CD45RO−CD27+CD8+ cells among CD8+ T cells being less than 15% (e.g., between 0.1% and 15%, e.g., between 0.1% and 12%, e.g., between 0.1% and 10%, e.g., between 0.1% and 8%) is indicative or predictive of one, two, or all of:
(a) decreased responsiveness of the subject to the BCMA CAR-expressing cell therapy;
(b) the subject as a non-responder of the BCMA CAR-expressing cell therapy; or
(c) decreased expansion of the BCMA CAR-expressing cell therapy in the subject, e.g., as measured by an assay disclosed herein, e.g., as measured by the copy number of CAR transgenes per μg DNA using qPCR.

17. The method of any one of claims 1-16, wherein the value for the proliferation of seeded cells from the subject during manufacturing of the BCMA CAR-expressing cell therapy comprises the fold expansion of seeded cells from the subject during manufacturing (e.g., total cell counts at the end of manufacturing relative to at the start of manufacturing) of the BCMA CAR-expressing cell therapy, e.g., as measured by an assay disclosed herein, e.g., as measured by cell counting.

18. The method of any one of claims 1-17, further comprising performing:
manufacturing the BCMA CAR-expressing cell therapy using cells (e.g., T cells) from the subject and administering the BCMA CAR-expressing cell therapy to the subject; or
administering, e.g., initiating administering or continuing administering, the BCMA CAR-expressing cell therapy to the subject, when:
(a) the subject was indicated or predicted to have increased responsiveness to the BCMA CAR-expressing cell therapy;
(b) the subject was indicated or predicted as a responder of the BCMA CAR-expressing cell therapy;
(c) the subject was indicated or predicted as suitable for the BCMA CAR-expressing cell therapy; or
(d) the BCMA CAR-expressing cell therapy was indicated or predicted to have increased expansion in the subject, e.g., as measured by an assay disclosed herein, e.g., as measured by the copy number of CAR transgenes per pg DNA using qPCR.

19. The method of any one of claims 1-17, further comprising performing one, two, three, four, five, six, seven, or all of:
administering an altered dosing regimen of the BCMA CAR-expressing cell therapy (e.g., a dosing regimen with a higher dose and/or more frequent administration than a reference dosing regimen) to the subject;
administering a second therapy (e.g., a second therapy that is not the BCMA CAR-expressing cell therapy) to the subject;
administering the BCMA CAR-expressing cell therapy and a second therapy to the subject;
discontinuing administration of the BCMA CAR-expressing cell therapy and optionally administering a second therapy to the subject;
modifying a manufacturing process of the BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;
modifying a manufacturing process of the BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;
modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or
administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject, when:
(a) the subject was indicated or predicted to have decreased responsiveness to the BCMA CAR-expressing cell therapy;
(b) the subject was indicated or predicted as a non-responder of the BCMA CAR-expressing cell therapy; or
(c) the BCMA CAR-expressing cell therapy was indicated or predicted to have decreased expansion in the subject, e.g., as measured by an assay disclosed herein, e.g., as measured by the copy number of CAR transgenes per pg DNA using qPCR.

20. A method of treating a subject having a disease associated with the expression of BCMA, comprising:
responsive to an increased value for one, two, three, four, five, or all of:
(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,
(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(iv) the level or activity of CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)), or
(vi) the proliferation of seeded cells from the subject during manufacturing of a BCMA CAR-expressing cell therapy,
as compared to a reference value, e.g., a non-responder reference value, performing:
manufacturing a BCMA CAR-expressing cell therapy using cells (e.g., T cells) from the subject and administering the BCMA CAR-expressing cell therapy to the subject; or
administering, e.g., initiating administering or continuing administering, a BCMA CAR-expressing cell therapy to the subject,
thereby treating the subject having the disease associated with the expression of BCMA.

21. A method of treating a subject having a disease associated with the expression of BCMA, comprising:
responsive to a decreased value for one, two, three, four, five, or all of:
(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,
(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(iv) the level or activity of CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)), or
(vi) the proliferation of seeded cells from the subject during manufacturing of a BCMA CAR-expressing cell therapy,
as compared to a reference value, e.g., a responder reference value, performing one, two, three, four, five, six, seven, or all of:
administering an altered dosing regimen of a BCMA CAR-expressing cell therapy (e.g., a dosing regimen with a higher dose and/or more frequent administration than a reference dosing regimen) to the subject;
administering a second therapy (e.g., a second therapy that is not a BCMA CAR-expressing cell therapy) to the subject;
administering a BCMA CAR-expressing cell therapy and a second therapy to the subject;
discontinuing administration of a BCMA CAR-expressing cell therapy and optionally administering a second therapy to the subject;
modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;
modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;
modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or
administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject,
thereby treating the subject having the disease associated with the expression of BCMA.

22. The method of claim 20, comprising: responsive to an increased value for one, two, three, four, five, or all of (i)-(vi), identifying or predicting one, two, three, or all of:
(a) the subject as having increased responsiveness to the BCMA CAR-expressing cell therapy;
(b) the subject as a responder of the BCMA CAR-expressing cell therapy;
(c) the subject as suitable for the BCMA CAR-expressing cell therapy; or
(d) the BCMA CAR-expressing cell therapy as having increased expansion in the subject, e.g., as measured by an assay disclosed herein, e.g., as measured by the copy number of CAR transgenes per pg DNA using qPCR.

23. The method of claim 21, comprising: response to a decreased value for one, two, three, four, five, or all of (i)-(vi), identifying or predicting one, two, or all of:
(a) the subject as having decreased responsiveness to the BCMA CAR-expressing cell therapy;
(b) the subject as a non-responder of the BCMA CAR-expressing cell therapy; or
(c) the BCMA CAR-expressing cell therapy as having decreased expansion in the subject, e.g., as measured by an assay disclosed herein, e.g., as measured by the copy number of CAR transgenes per pg DNA using qPCR.

24. The method of any one of claims 20-23, wherein the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry.

25. The method of claim 24, comprising:
responsive to the ratio being:
(1) greater than or equal to 1 (e.g., between 1 and 5, e.g., between 1 and 3.5); or
(2) greater than or equal to 1.6 (e.g., between 1.6 and 5, e.g., between 1.6 and 3.5), performing:
manufacturing a BCMA CAR-expressing cell therapy using cells (e.g., T cells) from the subject and administering the BCMA CAR-expressing cell therapy to the subject; or
administering, e.g., initiating administering or continuing administering, a BCMA CAR-expressing cell therapy to the subject.

26. The method of claim 24 or 25, comprising:
responsive to the ratio being less than 1 (e.g., between 0.001 and 1), performing one, two, three, four, five, six, seven, or all of:
administering an altered dosing regimen of a BCMA CAR-expressing cell therapy (e.g., a dosing regimen with a higher dose and/or more frequent administration than a reference dosing regimen) to the subject;
administering a second therapy (e.g., a second therapy that is not a BCMA CAR-expressing cell therapy) to the subject;
administering a BCMA CAR-expressing cell therapy and a second therapy to the subject;
discontinuing administration of a BCMA CAR-expressing cell therapy and optionally administering a second therapy to the subject;
modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;
modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;
modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or
administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject.

27. The method of any one of claims 20-26, wherein the value for the level or activity of CD8+ Tscm (stem cell memory T cells) comprises the percentage of CD8+ Tscm (stem cell memory T cells) among CD8+ T cells, e.g., as measured by an assay disclosed herein, e.g., flow cytometry.

28. The method of any one of claims 20-27, wherein the value for the level or activity of HLADR-CD95+CD27+CD8+ cells comprises the percentage of HLADR-CD95+CD27+CD8+ cells among CD8+ T cells, e.g., as measured by an assay disclosed herein, e.g., flow cytometry.

29. The method of claim 28, comprising:
responsive to the percentage of HLADR-CD95+CD27+CD8+ cells among CD8+ T cells being greater than or equal to 25% (e.g., between 30% and 90%, e.g., between 35% and 85%, e.g., between 40% and 80%, e.g., between 45% and 75%, e.g., between 50% and 75%), performing:
manufacturing a BCMA CAR-expressing cell therapy using cells (e.g., T cells) from the subject and administering the BCMA CAR-expressing cell therapy to the subject; or
administering, e.g., initiating administering or continuing administering, a BCMA CAR-expressing cell therapy to the subject.

30. The method of claim 28 or 29, comprising:
responsive to the percentage of HLADR-CD95+CD27+CD8+ cells among CD8+ T cells being less than 25% (e.g., between 0.1% and 25%, e.g., between 0.1% and 22%, e.g., between 0.1% and 20%, e.g., between 0.1% and 18%, e.g., between 0.1% and 15%), performing one, two, three, four, five, six, seven, or all of:
administering an altered dosing regimen of a BCMA CAR-expressing cell therapy (e.g., a dosing regimen with a higher dose and/or more frequent administration than a reference dosing regimen) to the subject;
administering a second therapy (e.g., a second therapy that is not a BCMA CAR-expressing cell therapy) to the subject;
administering a BCMA CAR-expressing cell therapy and a second therapy to the subject;
discontinuing administration of a BCMA CAR-expressing cell therapy and optionally administering a second therapy to the subject;
modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;
modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;
modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or
administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject.

31. The method of any one of claims 20-30, wherein the value for the level or activity of CD45RO−CD27+CD8+ cells comprises the percentage of CD45RO−CD27+CD8+ cells among CD8+ T cells, e.g., as measured by an assay disclosed herein, e.g., flow cytometry.

32. The method of claim 31, comprising:
responsive to the percentage of CD45RO−CD27+CD8+ cells among CD8+ T cells being greater than or equal to 20% (e.g., between 20% and 90%, e.g., between 20% and 80%, e.g., between 20% and 70%, e.g., between 20% and 60%), performing:
manufacturing a BCMA CAR-expressing cell therapy using cells (e.g., T cells) from the subject and administering the BCMA CAR-expressing cell therapy to the subject; or
administering, e.g., initiating administering or continuing administering, a BCMA CAR-expressing cell therapy to the subject.

33. The method of claim 31 or 32, comprising:
responsive to the percentage of CD45RO−CD27+CD8+ cells among CD8+ T cells being less than 20% (e.g., between 0.1% and 20%, e.g., between 0.1% and 18%, e.g., between 0.1% and 15%, e.g., between 0.1% and 12%, e.g., between 0.1% and 10%), performing one, two, three, four, five, six, seven, or all of:
administering an altered dosing regimen of a BCMA CAR-expressing cell therapy (e.g., a dosing regimen with a higher dose and/or more frequent administration than a reference dosing regimen) to the subject;
administering a second therapy (e.g., a second therapy that is not a BCMA CAR-expressing cell therapy) to the subject;
administering a BCMA CAR-expressing cell therapy and a second therapy to the subject;
discontinuing administration of a BCMA CAR-expressing cell therapy and optionally administering a second therapy to the subject;
modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;
modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;
modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or
administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject.

34. The method of any one of claims 20-33, wherein the value for the level or activity of CCR7+CD45RO−CD27+CD8+ cells comprises the percentage of CCR7+CD45RO−CD27+CD8+ cells among CD8+ T cells, e.g., as measured by an assay disclosed herein, e.g., flow cytometry.

35. The method of claim 34, comprising:
responsive to the percentage of CCR7+CD45RO−CD27+CD8+ cells among CD8+ T cells being greater than or equal to 15% (e.g., between 15% and 90%, e.g., between 15% and 80%, e.g., between 15% and 70%, e.g., between 15% and 60%, e.g., between 15% and 50%), performing:
manufacturing a BCMA CAR-expressing cell therapy using cells (e.g., T cells) from the subject and administering the BCMA CAR-expressing cell therapy to the subject; or
administering, e.g., initiating administering or continuing administering, a BCMA CAR-expressing cell therapy to the subject.

36. The method of claim 34 or 35, comprising:
responsive to the percentage of CCR7+CD45RO−CD27+CD8+ cells among CD8+ T cells being less than 15% (e.g., between 0.1% and 15%, e.g., between 0.1% and 12%, e.g., between 0.1% and 10%, e.g., between 0.1% and 8%), performing one, two, three, four, five, six, seven, or all of:
administering an altered dosing regimen of a BCMA CAR-expressing cell therapy (e.g., a dosing regimen with a higher dose and/or more frequent administration than a reference dosing regimen) to the subject;
administering a second therapy (e.g., a second therapy that is not a BCMA CAR-expressing cell therapy) to the subject;
administering a BCMA CAR-expressing cell therapy and a second therapy to the subject;
discontinuing administration of a BCMA CAR-expressing cell therapy and optionally administering a second therapy to the subject;
modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;
modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;
modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or
administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject.

37. The method of any one of claims 20-36, wherein the value for the proliferation of seeded cells from the subject during manufacturing of the BCMA CAR-expressing cell therapy comprises the fold expansion of seeded cells from the subject during manufacturing (e.g., total cell counts at the end of manufacturing relative to at the start of manufacturing) of the BCMA CAR-expressing cell therapy, e.g., as measured by an assay disclosed herein, e.g., as measured by cell counting.

38. A method of evaluating or predicting the potency of a BCMA CAR-expressing cell therapy in a subject, wherein the subject has a disease associated with the expression of BCMA and wherein the BCMA CAR-expressing cell therapy is manufactured using cells (e.g., T cells) from the subject, comprising:
acquiring a value for one, two, three, four, five, or all of:
(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,
(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(iv) the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)), or
(vi) the proliferation of seeded cells from the subject during manufacturing of the BCMA CAR-expressing cell therapy, e.g., as measured by population doublings by day 9 (PDL9), wherein:
(a) an increase in the value of one, two, three, four, five, or all of (i)-(vi), as compared to a reference value, e.g., a non-responder reference value, is indicative or predictive of increased potency of the BCMA CAR-expressing cell therapy in the subject; or
(b) a decrease in the value of one, two, three, four, five, or all of (i)-(vi), as compared to a reference value, e.g., a responder reference value, is indicative or predictive of decreased potency of the BCMA CAR-expressing cell therapy in the subject,
thereby evaluating or predicting the potency of the BCMA CAR-expressing cell therapy.

39. The method of claim 38, wherein the increase in the value of one, two, three, four, five, or all of (i)-(vi), as compared to a reference value, e.g., a non-responder reference value, is indicative or predictive of increased expansion of the BCMA CAR-expressing cell therapy in the subject, e.g., as measured by an assay disclosed herein, e.g., as measured by the copy number of CAR transgenes per μg DNA using qPCR.

40. The method of claim 38 or 39, wherein the decrease in the value of one, two, three, four, five, or all of (i)-(vi), as compared to a reference value, e.g., a responder reference value, is indicative or predictive of decreased expansion of the BCMA CAR-expressing cell therapy in the subject, e.g., as measured by an assay disclosed herein, e.g., as measured by the copy number of CAR transgenes per μg DNA using qPCR.

41. A method of manufacturing a BCMA CAR-expressing cell therapy, wherein the BCMA CAR-expressing cell therapy is manufactured using cells (e.g., T cells) from a subject, comprising:
acquiring a value for one, two, three, four, five, or all of:
(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,
(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(iv) the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)), or
(vi) the proliferation of seeded cells from the subject during manufacturing of the BCMA CAR-expressing cell therapy, e.g., as measured by population doublings by day 9 (PDL9), wherein:
responsive to an increase in the value of one, two, three, four, five, or all of (i)-(vi), as compared to a reference value, e.g., a non-responder reference value, manufacturing the BCMA CAR-expressing cell therapy using cells from the subject.

42. A method of manufacturing a BCMA CAR-expressing cell therapy, wherein the BCMA CAR-expressing cell therapy is manufactured using cells (e.g., T cells) from a subject, comprising:
acquiring a value for one, two, three, four, five, or all of:
(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,
(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(iv) the level or activity of CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)),
(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of the BCMA CAR-expressing cell therapy (e.g., a leukapheresis sample after monocytes are removed using elutriation)), or
(vi) the proliferation of seeded cells from the subject during manufacturing of the BCMA CAR-expressing cell therapy, e.g., as measured by population doublings by day 9 (PDL9), wherein:
responsive to a decrease in the value of one, two, three, four, five, or all of (i)-(vi), as compared to a reference value, e.g., a responder reference value, performing one, two, three, or all of:
modifying a manufacturing process of the BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR;
modifying a manufacturing process of the BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR;
modifying a manufacturing process of the BCMA CAR-expressing cell therapy, e.g., increasing the proliferation of seeded cells from the subject during the manufacturing of the BCMA CAR-expressing cell therapy; or
administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), in a seed culture at the start of the manufacturing of the BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), and manufacturing the BCMA CAR-expressing cell therapy using cells (e.g., T cells) from the subject.

43. The method of any one of claims 38-42, wherein the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry.

44. The method of any one of claims 38-43, wherein the value for the level or activity of CD8+ Tscm (stem cell memory T cells) comprises the percentage of CD8+ Tscm (stem cell memory T cells) among CD8+ T cells, e.g., as measured by an assay disclosed herein, e.g., flow cytometry.

45. The method of any one of claims 38-44, wherein the value for the level or activity of HLADR-CD95+CD27+CD8+ cells comprises the percentage of HLADR-CD95+CD27+CD8+ cells among CD8+ T cells, e.g., as measured by an assay disclosed herein, e.g., flow cytometry.

46. The method of any one of claims 38-45, the value for the level or activity of CD45RO−CD27+CD8+ cells comprises the percentage of CD45RO−CD27+CD8+ cells among CD8+ T cells, e.g., as measured by an assay disclosed herein, e.g., flow cytometry.

47. The method of any one of claims 38-46, wherein the value for the level or activity of CCR7+CD45RO−CD27+CD8+ cells comprises the percentage of CCR7+CD45RO−CD27+CD8+ cells among CD8+ T cells, e.g., as measured by an assay disclosed herein, e.g., flow cytometry.

48. The method of any one of claims 38-47, the value for the proliferation of seeded cells from the subject during manufacturing of the BCMA CAR-expressing cell therapy comprises the fold expansion of seeded cells from the subject during manufacturing (e.g., total cell counts at the end of manufacturing relative to at the start of manufacturing) of the BCMA CAR-expressing cell therapy, e.g., as measured by an assay disclosed herein, e.g., as measured by cell counting.

49. The method of any one of claims 1-48, comprising administering the BCMA CAR-expressing cell therapy to the subject in combination with one, two, or all of:
(1) an agent that increases the efficacy of the cell comprising the CAR nucleic acid or CAR polypeptide;
(2) an agent that ameliorates one or more side effects associated with administration of the cell comprising the CAR nucleic acid or CAR polypeptide;
(3) an agent that treats the disease associated with the expression of BCMA.

50. The method of any one of claims 1-49, comprising administering the BCMA CAR-expressing cell therapy to the subject in combination with a compound of Formula (I) (COF1), wherein the COF1 is: 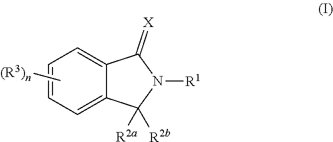
or a pharmaceutically acceptable salt, ester, hydrate, solvate, or tautomer thereof, wherein:
X is O or S;
R¹ is C₁-C₆ alkyl, C₂-C₆ alkenyl, C₂-C₆ alkynyl, C₁-C₆ heteroalkyl, carbocyclyl, heterocyclyl, aryl, or heteroaryl, each of which is optionally substituted by one or more R⁴;
each of R^{2a} and R^{2b} is independently hydrogen or C₁-C₆ alkyl; or R^{2a} and R^{2b} together with the carbon atom to which they are attached form a carbonyl group or a thiocarbonyl group;
each of R³ is independently C₁-C₆ alkyl, C₂-C₆ alkenyl, C₂-C₆ alkynyl, C₁-C₆ heteroalkyl, halo, cyano, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)ₓN(R^{C})(R^{D}), or —N(R^{C})S(O)ₓR^{E}, wherein each alkyl, alkenyl, alkynyl, and heteroalkyl is independently and optionally substituted with one or more R⁶;
each R⁴ is independently C₁-C₆ alkyl, C₂-C₆ alkenyl, C₂-C₆ alkynyl, C₁-C₆ heteroalkyl, halo, cyano, oxo, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)ₓN(R^{C})(R^{D}), —N(R^{C})S(O)ₓR^{E}, carbocyclyl, heterocyclyl, aryl, or heteroaryl, wherein each alkyl, alkenyl, alkynyl, heteroalkyl, carbocyclyl, heterocyclyl, aryl, and heteroaryl is independently and optionally substituted with one or more IV;
each of R^{A}, R^{B}, R^{C}, R^{D}, and R^{E} is independently hydrogen or C₁-C₆ alkyl;
each R⁶ is independently C₁-C₆ alkyl, oxo, cyano, —OR^{B}, —N(R^{C})(R^{D}), —C(O)N(R^{C})(R^{D}), —N(R^{C})C(O)R^{A}, aryl, or heteroaryl, wherein each aryl and heteroaryl is independently and optionally substituted with one or more R⁸;
each R⁷ is independently halo, oxo, cyano, —OR^{B}, —N(R^{C})(R^{D}), —C(O)N(R^{C})(R^{D}), or —N(R^{C})C(O)R^{A};
each R⁸ is independently C₁-C₆ alkyl, cyano, —OR^{B}, —N(R^{C})(R^{D}), —C(O)N(R^{C})(R^{D}), or —N(R^{C})C(O)R^{A};
n is 0, 1, 2, 3 or 4; and
x is 0, 1, or 2, optionally wherein:
(1) the COF1 is an immunomodulatory imide drug (IMiD), or a pharmaceutically acceptable salt thereof;
(2) the COF1 is selected from the group consisting of lenalidomide, pomalidomide, thalidomide, and 2-(4-(tert-butyl)phenyl)-N-((2-(2,6-dioxopiperidin-3-yl)-1-oxoisoindolin-5-yl)methyl)acetamide, or a pharmaceutically acceptable salt thereof;
(3) the COF1 is selected from the group consisting of: 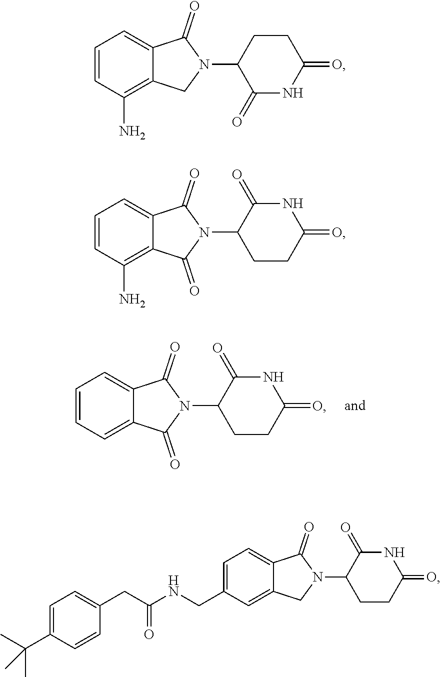 [SMILES: Nc1cccc2c1CN(C1CCC(=O)NC1=O)C2=O ; Nc1cccc2c1C(=O)N(C1CCC(=O)NC1=O)C2=O ; O=C1CCC(N2C(=O)c3ccccc3C2=O)C(=O)N1 ; CC(C)(C)c1ccc(CC(=O)NCc2ccc3c(c2)CN(C2CCC(=O)NC2=O)C3=O)cc1]
or a pharmaceutically acceptable salt thereof; or
(4) the COF1 is lenalidomide, or a pharmaceutically acceptable salt thereof.

51. The method of any one of claims 1-50, comprising administering the BCMA CAR-expressing cell therapy to the subject in combination with a kinase inhibitor, e.g., a BTK inhibitor, e.g., ibrutinib.

52. The method of any one of claims 1-51, comprising administering the BCMA CAR-expressing cell therapy to the subject in combination with a second CAR-expressing cell therapy, optionally wherein the second CAR-expressing cell therapy is:
(1) a CD19 CAR-expressing cell therapy, e.g., a CD19 CAR-expressing cell therapy disclosed herein, e.g., CTL119 or CTL019, optionally wherein the CD19 CAR-expressing cell therapy is administered after the administration of the BCMA CAR-expressing cell therapy, e.g., after CD19 expression is increased in the subject following the administration of the BCMA CAR-expressing cell therapy;
(2) a CD20 CAR-expressing cell therapy, e.g., a CD20 CAR-expressing cell therapy disclosed herein, optionally wherein the CD20 CAR-expressing cell therapy is administered after the administration of the BCMA CAR-expressing cell therapy, e.g., after CD20 expression is increased in the subject following the administration of the BCMA CAR-expressing cell therapy;
(3) a CD22 CAR-expressing cell therapy, e.g., a CD22 CAR-expressing cell therapy disclosed herein, optionally wherein the CD22 CAR-expressing cell therapy is administered after the administration of the BCMA CAR-expressing cell therapy, e.g., after CD22 expression is increased in the subject following the administration of the BCMA CAR-expressing cell therapy;
(4) a CAR-expressing cell therapy comprising a cell expressing a first CAR and a second CAR, wherein the first CAR is a BCMA CAR (e.g., a BCMA CAR disclosed herein), optionally wherein the second CAR is selected from the group consisting of a CD19 CAR (e.g., a CD19 CAR disclosed herein), a CD20 CAR (e.g., a CD20 CAR disclosed herein), and a CD22 CAR (e.g., a CD22 CAR disclosed herein); or
(5) a CAR-expressing cell therapy comprising a cell expressing a multispecific CAR, e.g., a bispecific CAR, that binds to a first antigen and a second antigen, wherein the first antigen is BCMA, optionally wherein the second antigen is selected from the group consisting of CD19, CD20, and CD22.

53. The method of any one of claims 1-52, comprising administering the BCMA CAR-expressing cell therapy to the subject in combination with a CD19 inhibitor, e.g., a CD19 inhibitor disclosed herein, optionally wherein the CD19 inhibitor is administered after the administration of the BCMA CAR-expressing cell therapy, e.g., after CD19 expression is increased in the subject following the administration of the BCMA CAR-expressing cell therapy.

54. The method of any one of claims 1-53, comprising administering the BCMA CAR-expressing cell therapy to the subject in combination with a CD20 inhibitor, e.g., a CD20 inhibitor disclosed herein, optionally wherein the CD20 inhibitor is a multispecific antibody molecule, e.g., a bispecific antibody molecule, that binds to CD20 and CD3, e.g., THG338, optionally wherein the CD20 inhibitor is administered after the administration of the BCMA CAR-expressing cell therapy, e.g., after CD20 expression is increased in the subject following the administration of the BCMA CAR-expressing cell therapy.

55. The method of any one of claims 1-54, comprising administering the BCMA CAR-expressing cell therapy to the subject in combination with a CD22 inhibitor, e.g., a CD22 inhibitor disclosed herein, optionally wherein the CD22 inhibitor is administered after the administration of the BCMA CAR-expressing cell therapy, e.g., after CD22 expression is increased in the subject following the administration of the BCMA CAR-expressing cell therapy.

56. The method of any one of claims 1-55, comprising administering the BCMA CAR-expressing cell therapy to the subject in combination with a molecule that binds to Fc receptor like 2 (FCRL2) or Fc receptor like 5 (FCRL5), optionally wherein the molecule is:
(1) a CAR-expressing cell therapy comprising a cell expressing a CAR that binds to FCRL2 or FCRL5; or
(2) a multispecific antibody molecule, e.g., a bispecific antibody molecule, that binds to a first antigen and a second antigen, wherein the first antigen is FCRL2 or FCRL5, optionally wherein the second antigen is CD3.

57. The method of any one of claims 1-56, comprising administering the BCMA CAR-expressing cell therapy to the subject in combination with an interleukin-15 (IL-15) polypeptide, an interleukin-15 receptor alpha (IL-15Ra) polypeptide, or a combination of both an IL-15 polypeptide and an IL-15Ra polypeptide, e.g., hetIL-15.

58. The method of any one of claims 1-57, comprising administering the BCMA CAR-expressing cell therapy to the subject in combination with an inhibitor of TGF beta.

59. The method of any one of claims 1-58, comprising administering the BCMA CAR-expressing cell therapy to the subject in combination with an EGFR^{mut}-tyrosine kinase inhibitor (TKI), e.g., EGF816.

60. The method of any one of claims 1-59, comprising administering the BCMA CAR-expressing cell therapy to the subject in combination with an adenosine A2AR antagonist, optionally wherein:
(1) the adenosine A2AR antagonist is selected from the group consisting of PBF509, CPI444, AZD4635, Vipadenant, GBV-2034, and AB928; or
(2) the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-c]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine.

61. The method of any one of claims 1-60, comprising administering the BCMA CAR-expressing cell therapy to the subject in combination with an anti-CD73 antibody molecule, e.g., an anti-CD73 antibody molecule disclosed herein.

62. The method of any one of claims 1-61, comprising administering the BCMA CAR-expressing cell therapy to the subject in combination with a check point inhibitor, optionally wherein the check point inhibitor is:
(1) a PD-1 inhibitor, optionally wherein the PD-1 inhibitor is selected from the group consisting of PDR001, Nivolumab, Pembrolizumab, Pidilizumab, MEDI0680, REGN2810, TSR-042, PF-06801591, and AMP-224, optionally wherein the PD-1 inhibitor increases expansion of BCMA CAR-expressing cells in the subject;
(2) a PD-L1 inhibitor, optionally wherein the PD-L1 inhibitor is selected from the group consisting of FAZ053, Atezolizumab, Avelumab, Durvalumab, and BMS-936559, optionally wherein the PD-L1 inhibitor increases expansion of BCMA CAR-expressing cells in the subject;
(3) a LAG-3 inhibitor, optionally wherein the LAG-3 inhibitor is selected from the group consisting of LAG525, BMS-986016, TSR-033, MK-4280 and REGN3767; or
(4) a TIM-3 inhibitor, optionally wherein the TIM-3 inhibitor is selected from the group consisting of MGB453, TSR-022, and LY3321367.

63. The method of any one of claims 1-62, comprising administering the BCMA CAR-expressing cell therapy to the subject in combination with an antibody molecule that binds to CD32B.

64. The method of any one of claims 1-63, comprising administering the BCMA CAR-expressing cell therapy to the subject in combination with an antibody molecule that binds to IL-17, e.g., an antagonistic antibody molecule that binds to IL-17, e.g., CJM112.

65. The method of any one of claims 1-64, comprising administering the BCMA CAR-expressing cell therapy to the subject in combination with an antibody molecule that binds to IL-1 beta.

66. The method of any one of claims 1-65, comprising administering the BCMA CAR-expressing cell therapy to the subject in combination with an inhibitor of indoleamine 2,3-dioxygenase (IDO) and/or tryptophan 2,3-dioxygenase (TDO), e.g., an IDO1 inhibitor, optionally wherein the inhibitor of IDO and/or TDO is chosen from:
(1) INCB24360, indoximod, NLG919, epacadostat, NLG919, or F001287; or
(2) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan.

67. A method of treating a subject having a disease associated with the expression of BCMA, comprising administering a BCMA CAR-expressing cell therapy and a second therapy to the subject, wherein the second therapy is:
(1) a CD19 CAR-expressing cell therapy, e.g., a CD19 CAR-expressing cell therapy disclosed herein, e.g., CTL119 or CTL019, optionally wherein the CD19 CAR-expressing cell therapy is administered after the administration of the BCMA CAR-expressing cell therapy, e.g., after CD19 expression is increased in the subject following the administration of the BCMA CAR-expressing cell therapy;
(2) a CD20 CAR-expressing cell therapy, e.g., a CD20 CAR-expressing cell therapy disclosed herein, optionally wherein the CD20 CAR-expressing cell therapy is administered after the administration of the BCMA CAR-expressing cell therapy, e.g., after CD20 expression is increased in the subject following the administration of the BCMA CAR-expressing cell therapy;
(3) a CD22 CAR-expressing cell therapy, e.g., a CD22 CAR-expressing cell therapy disclosed herein, optionally wherein the CD22 CAR-expressing cell therapy is administered after the administration of the BCMA CAR-expressing cell therapy, e.g., after CD22 expression is increased in the subject following the administration of the BCMA CAR-expressing cell therapy;
(4) a CAR-expressing cell therapy comprising a cell expressing a first CAR and a second CAR, wherein the first CAR is a BCMA CAR (e.g., a BCMA CAR disclosed herein), optionally wherein the second CAR is selected from the group consisting of a CD19 CAR (e.g., a CD19 CAR disclosed herein), a CD20 CAR (e.g., a CD20 CAR disclosed herein), and a CD22 CAR (e.g., a CD22 CAR disclosed herein); or
(5) a CAR-expressing cell therapy comprising a cell expressing a multispecific CAR, e.g., a bispecific CAR that binds to a first antigen and a second antigen, wherein the first antigen is BCMA, optionally wherein the second antigen is selected from the group consisting of CD19, CD20, and CD22.

68. A method of treating a subject having a disease associated with the expression of BCMA, comprising administering a BCMA CAR-expressing cell therapy and a second therapy to the subject, wherein the second therapy is:
(1) a CD19 inhibitor, e.g., a CD19 inhibitor disclosed herein, optionally wherein the CD19 inhibitor is administered after the administration of the BCMA CAR-expressing cell therapy, e.g., after CD19 expression is increased in the subject following the administration of the BCMA CAR-expressing cell therapy;
(2) a CD20 inhibitor, e.g., a CD20 inhibitor disclosed herein, optionally wherein the CD20 inhibitor is a multispecific antibody molecule, e.g., a bispecific antibody molecule that binds to CD20 and CD3, e.g., THG338, optionally wherein the CD20 inhibitor is administered after the administration of the BCMA CAR-expressing cell therapy, e.g., after CD20 expression is increased in the subject following the administration of the BCMA CAR-expressing cell therapy; or
(3) a CD22 inhibitor, e.g., a CD22 inhibitor disclosed herein, optionally wherein the CD22 inhibitor is administered after the administration of the BCMA CAR-expressing cell therapy, e.g., after CD22 expression is increased in the subject following the administration of the BCMA CAR-expressing cell therapy.

69. A method of treating a subject having a disease associated with the expression of BCMA, comprising administering a BCMA CAR-expressing cell therapy and a second therapy to the subject, wherein the second therapy is a molecule that binds to Fc receptor like 2 (FCRL2) or Fc receptor like 5 (FCRL5), optionally wherein the molecule is:
(1) a CAR-expressing cell therapy comprising a cell expressing a CAR that binds to FCRL2 or FCRL5; or
(2) a multispecific antibody molecule, e.g., a bispecific antibody molecule, that binds to a first antigen and a second antigen, wherein the first antigen is FCRL2 or FCRL5, optionally wherein the second antigen is CD3.

70. A method of treating a subject having a disease associated with the expression of BCMA, comprising administering a BCMA CAR-expressing cell therapy and a second therapy to the subject, wherein the second therapy is an inhibitor of TGF beta.

71. A method of treating a subject having a disease associated with the expression of BCMA, comprising administering a BCMA CAR-expressing cell therapy and a second therapy to the subject, wherein the second therapy is an EGFR′-tyrosine kinase inhibitor (TKI), e.g., EGF816.

72. A method of treating a subject having a disease associated with the expression of BCMA, comprising administering a BCMA CAR-expressing cell therapy and a second therapy to the subject, wherein the second therapy is an adenosine A2AR antagonist, optionally wherein:
(1) the adenosine A2AR antagonist is selected from the group consisting of PBF509, CPI444, AZD4635, Vipadenant, GBV-2034, and AB928; or
(2) the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-c]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine.

73. A method of treating a subject having a disease associated with the expression of BCMA, comprising administering a BCMA CAR-expressing cell therapy and a second therapy to the subject, wherein the second therapy is an anti-CD73 antibody molecule, e.g., an anti-CD73 antibody molecule disclosed herein.

74. A method of treating a subject having a disease associated with the expression of BCMA, comprising administering a BCMA CAR-expressing cell therapy and a second therapy to the subject, wherein the second therapy is a check point inhibitor, optionally wherein the check point inhibitor is:
(1) a PD-1 inhibitor, optionally wherein the PD-1 inhibitor is selected from the group consisting of PDR001, Nivolumab, Pembrolizumab, Pidilizumab, MEDI0680, REGN2810, TSR-042, PF-06801591, and AMP-224, optionally wherein the PD-1 inhibitor is administered after the administration of the BCMA CAR-expressing cell therapy, e.g., after the expression of PD-1 or PD-L1 is increased in the subject following the administration of the BCMA CAR-expressing cell therapy, optionally wherein the PD-1 inhibitor increases expansion of BCMA CAR-expressing cells in the subject;
(2) a PD-L1 inhibitor, optionally wherein the PD-L1 inhibitor is selected from the group consisting of FAZ053, Atezolizumab, Avelumab, Durvalumab, and BMS-936559, optionally wherein the PD-L1 inhibitor is administered after the administration of the BCMA CAR-expressing cell therapy, e.g., after the expression of PD-1 or PD-L1 is increased in the subject following the administration of the BCMA CAR-expressing cell therapy, optionally wherein the PD-L1 inhibitor increases expansion of BCMA CAR-expressing cells in the subject;
(3) a LAG-3 inhibitor, optionally wherein the LAG-3 inhibitor is selected from the group consisting of LAG525, BMS-986016, TSR-033, MK-4280 and REGN3767, optionally wherein the LAG-3 inhibitor is administered after the administration of the BCMA CAR-expressing cell therapy, e.g., after the expression of LAG-3 is increased in the subject following the administration of the BCMA CAR-expressing cell therapy; or
(4) a TIM-3 inhibitor, optionally wherein the TIM-3 inhibitor is selected from the group consisting of MGB453, TSR-022, and LY3321367, optionally wherein the TIM-3 inhibitor is administered after the administration of the BCMA CAR-expressing cell therapy, e.g., after the expression of TIM-3 is increased in the subject following the administration of the BCMA CAR-expressing cell therapy.

75. A method of treating a subject having a disease associated with the expression of BCMA, comprising administering a BCMA CAR-expressing cell therapy and a second therapy to the subject, wherein the second therapy is an antibody molecule that binds to CD32B.

76. A method of treating a subject having a disease associated with the expression of BCMA, comprising administering a BCMA CAR-expressing cell therapy and a second therapy to the subject, wherein the second therapy is an antibody molecule that binds to IL-17, e.g., an antagonistic antibody molecule that binds to IL-17, e.g., CJM112.

77. A method of treating a subject having a disease associated with the expression of BCMA, comprising administering a BCMA CAR-expressing cell therapy and a second therapy to the subject, wherein the second therapy is an antibody molecule that binds to IL-1 beta.

78. A method of treating a subject having a disease associated with the expression of BCMA, comprising administering a BCMA CAR-expressing cell therapy and a second therapy to the subject, wherein the second therapy is an inhibitor of indoleamine 2,3-dioxygenase (IDO) and/or tryptophan 2,3-dioxygenase (TDO), e.g., an IDO1 inhibitor, optionally wherein the inhibitor of IDO and/or TDO is chosen from:
(1) INCB24360, indoximod, NLG919, epacadostat, NLG919, or F001287; or
(2) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan, optionally wherein:
the inhibitor of IDO and/or TDO is administered after the administration of the BCMA CAR-expressing cell therapy, e.g., after IDO and/or TDO expression is increased in the subject following the administration of the BCMA CAR-expressing cell therapy.

79. The method of any one of claims 67-78, wherein the second therapy is administered prior to, concurrently with, or subsequent to the administration of the BCMA CAR-expressing cell therapy.

80. A method of treating a subject having a disease associated with the expression of BCMA, wherein the subject has received or is receiving a BCMA CAR-expressing cell therapy, comprising:
responsive to an increase in a value of the level or activity of an antigen in the subject, e.g., in a sample from the subject (e.g., a biopsy sample, e.g., a bone marrow biopsy sample), at at least one time point after the subject began receiving the BCMA CAR-expressing cell therapy, relative to a reference value, wherein the reference value is:
(i) the level or activity of the antigen in the subject, e.g., in a sample from the subject (e.g., a biopsy sample, e.g., a bone marrow biopsy sample), prior to the at least one time point (e.g., the level or activity of the antigen in the subject before the subject began receiving the BCMA CAR-expressing cell therapy, or the level or activity of the antigen in the subject after the subject began receiving the BCMA CAR-expressing cell therapy but prior to the at least one time point);
(ii) the level or activity of the antigen in a different subject having the disease associated with the expression of BCMA; or
(iii) an average level or activity of the antigen in a population of subjects having the disease associated with the expression of BCMA,
administering an inhibitor of the antigen to the subject, wherein:
(1) the antigen is CD19 and the inhibitor of the antigen is a CD19 inhibitor, optionally wherein the CD19 inhibitor is:
(a) a CD19 CAR-expressing cell therapy, e.g., a CD19 CAR-expressing cell therapy disclosed herein, e.g., CTL119 or CTL019;
(b) a CAR-expressing cell therapy comprising a cell expressing a first CAR and a second CAR, wherein the first CAR is a BCMA CAR (e.g., a BCMA CAR disclosed herein) and the second CAR is a CD19 CAR (e.g., a CD19 CAR disclosed herein); or
(c) a CAR-expressing cell therapy comprising a cell expressing a multispecific CAR, e.g., a bispecific CAR, that binds to BCMA and CD19,
(2) the antigen is CD20 and the inhibitor of the antigen is a CD20 inhibitor, optionally wherein the CD20 inhibitor is:
(d) a CD20 CAR-expressing cell therapy, e.g., a CD20 CAR-expressing cell therapy disclosed herein;
(e) a CAR-expressing cell therapy comprising a cell expressing a first CAR and a second CAR, wherein the first CAR is a BCMA CAR (e.g., a BCMA CAR disclosed herein) and the second CAR is a CD20 CAR (e.g., a CD20 CAR disclosed herein);
(f) a CAR-expressing cell therapy comprising a cell expressing a multispecific CAR, e.g., a bispecific CAR, that binds to BCMA and CD20; or
(g) a multispecific antibody molecule, e.g., a bispecific antibody molecule, that binds to CD20 and CD3, e.g., THG338,
(3) the antigen is CD22 and the inhibitor of the antigen is a CD22 inhibitor, optionally wherein the CD22 inhibitor is:
(h) a CD22 CAR-expressing cell therapy, e.g., a CD22 CAR-expressing cell therapy disclosed herein;
(i) a CAR-expressing cell therapy comprising a cell expressing a first CAR and a second CAR, wherein the first CAR is a BCMA CAR (e.g., a BCMA CAR disclosed herein) and the second CAR is a CD22 CAR (e.g., a CD22 CAR disclosed herein); or
(j) a CAR-expressing cell therapy comprising a cell expressing a multispecific CAR, e.g., a bispecific CAR, that binds to BCMA and CD22,
(4) the antigen is PD1 or PD-L1 and the inhibitor of the antigen is an anti-PD1 antibody molecule or an anti-PD-L1 antibody molecule, optionally wherein the inhibitor of the antigen is:
(k) PDR001, Nivolumab, Pembrolizumab, Pidilizumab, MEDI0680, REGN2810, TSR-042, PF-06801591, or AMP-224; or
(l) FAZ053, Atezolizumab, Avelumab, Durvalumab, or BMS-936559,
(5) the antigen is IDO or TDO and the inhibitor of the antigen is an inhibitor of IDO and/or TDO, optionally wherein the inhibitor of IDO and/or TDO is:
(m) INCB24360, indoximod, NLG919, epacadostat, NLG919, or F001287; or
(n) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan, or
(6) the antigen is TGF-beta and the inhibitor of the antigen is a TGF beta inhibitor.

81. A method of treating a subject having a disease associated with the expression of BCMA, wherein the subject has received or is receiving a BCMA CAR-expressing cell therapy, comprising:
acquiring a value of the level or activity of an antigen in the subject, e.g., in a sample from the subject (e.g., a biopsy sample, e.g., a bone marrow biopsy sample), at at least one time point after the subject began receiving the BCMA CAR-expressing cell therapy,
responsive to an increase in the value relative to a reference value, wherein the reference value is:
(i) the level or activity of the antigen in the subject, e.g., in a sample from the subject (e.g., a biopsy sample, e.g., a bone marrow biopsy sample), prior to the at least one time point (e.g., the level or activity of the antigen in the subject before the subject began receiving the BCMA CAR-expressing cell therapy, or the level or activity of the antigen in the subject after the subject began receiving the BCMA CAR-expressing cell therapy but prior to the at least one time point);
(ii) the level or activity of the antigen in a different subject having the disease associated with the expression of BCMA; or
(iii) an average level or activity of the antigen in a population of subjects having the disease associated with the expression of BCMA,
administering an inhibitor of the antigen to the subject, wherein:
(1) the antigen is CD19 and the inhibitor of the antigen is a CD19 inhibitor, optionally wherein the CD19 inhibitor is:
(a) a CD19 CAR-expressing cell therapy, e.g., a CD19 CAR-expressing cell therapy disclosed herein, e.g., CTL119 or CTL019;
(b) a CAR-expressing cell therapy comprising a cell expressing a first CAR and a second CAR, wherein the first CAR is a BCMA CAR (e.g., a BCMA CAR disclosed herein) and the second CAR is a CD19 CAR (e.g., a CD19 CAR disclosed herein); or
(c) a CAR-expressing cell therapy comprising a cell expressing a multispecific CAR, e.g., a bispecific CAR, that binds to BCMA and CD19,
(2) the antigen is CD20 and the inhibitor of the antigen is a CD20 inhibitor, optionally wherein the CD20 inhibitor is:
(d) a CD20 CAR-expressing cell therapy, e.g., a CD20 CAR-expressing cell therapy disclosed herein;
(e) a CAR-expressing cell therapy comprising a cell expressing a first CAR and a second CAR, wherein the first CAR is a BCMA CAR (e.g., a BCMA CAR disclosed herein) and the second CAR is a CD20 CAR (e.g., a CD20 CAR disclosed herein);
(f) a CAR-expressing cell therapy comprising a cell expressing a multispecific CAR, e.g., a bispecific CAR, that binds to BCMA and CD20; or
(g) a multispecific antibody molecule, e.g., a bispecific antibody molecule, that binds to CD20 and CD3, e.g., THG338,
(3) the antigen is CD22 and the inhibitor of the antigen is a CD22 inhibitor, optionally wherein the CD22 inhibitor is:
(h) a CD22 CAR-expressing cell therapy, e.g., a CD22 CAR-expressing cell therapy disclosed herein;
(i) a CAR-expressing cell therapy comprising a cell expressing a first CAR and a second CAR, wherein the first CAR is a BCMA CAR (e.g., a BCMA CAR disclosed herein) and the second CAR is a CD22 CAR (e.g., a CD22 CAR disclosed herein); or
(j) a CAR-expressing cell therapy comprising a cell expressing a multispecific CAR, e.g., a bispecific CAR, that binds to BCMA and CD22,
(4) the antigen is PD1 or PD-L1 and the inhibitor of the antigen is an anti-PD1 antibody molecule or an anti-PD-L1 antibody molecule, optionally wherein the inhibitor of the antigen is:
(k) PDR001, Nivolumab, Pembrolizumab, Pidilizumab, MEDI0680, REGN2810, TSR-042, PF-06801591, or AMP-224; or
(l) FAZ053, Atezolizumab, Avelumab, Durvalumab, or BMS-936559,
(5) the antigen is IDO or TDO and the inhibitor of the antigen is an inhibitor of IDO and/or TDO, optionally wherein the inhibitor of IDO and/or TDO is:
(m) INCB24360, indoximod, NLG919, epacadostat, NLG919, or F001287; or
(n) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan, or
(6) the antigen is TGF-beta and the inhibitor of the antigen is a TGF beta inhibitor.

82. A method of treating a subject having a disease associated with the expression of BCMA, comprising:
administering a BCMA CAR-expressing cell therapy to the subject,
responsive to an increase in a value of the level or activity of an antigen in the subject, e.g., in a sample from the subject (e.g., a biopsy sample, e.g., a bone marrow biopsy sample), at at least one time point after the subject began receiving the BCMA CAR-expressing cell therapy, relative to a reference value, wherein the reference value is:
(i) the level or activity of the antigen in the subject, e.g., in a sample from the subject (e.g., a biopsy sample, e.g., a bone marrow biopsy sample), prior to the at least one time point (e.g., the level or activity of the antigen in the subject before the subject began receiving the BCMA CAR-expressing cell therapy, or the level or activity of the antigen in the subject after the subject began receiving the BCMA CAR-expressing cell therapy but prior to the at least one time point);
(ii) the level or activity of the antigen in a different subject having the disease associated with the expression of BCMA; or
(iii) an average level or activity of the antigen in a population of subjects having the disease associated with the expression of BCMA,
administering an inhibitor of the antigen to the subject, wherein:
(1) the antigen is CD19 and the inhibitor of the antigen is a CD19 inhibitor, optionally wherein the CD19 inhibitor is:
(a) a CD19 CAR-expressing cell therapy, e.g., a CD19 CAR-expressing cell therapy disclosed herein, e.g., CTL119 or CTL019;
(b) a CAR-expressing cell therapy comprising a cell expressing a first CAR and a second CAR, wherein the first CAR is a BCMA CAR (e.g., a BCMA CAR disclosed herein) and the second CAR is a CD19 CAR (e.g., a CD19 CAR disclosed herein); or
(c) a CAR-expressing cell therapy comprising a cell expressing a multispecific CAR, e.g., a bispecific CAR, that binds to BCMA and CD19,
(2) the antigen is CD20 and the inhibitor of the antigen is a CD20 inhibitor, optionally wherein the CD20 inhibitor is:
(d) a CD20 CAR-expressing cell therapy, e.g., a CD20 CAR-expressing cell therapy disclosed herein;
(e) a CAR-expressing cell therapy comprising a cell expressing a first CAR and a second CAR, wherein the first CAR is a BCMA CAR (e.g., a BCMA CAR disclosed herein) and the second CAR is a CD20 CAR (e.g., a CD20 CAR disclosed herein);
(f) a CAR-expressing cell therapy comprising a cell expressing a multispecific CAR, e.g., a bispecific CAR, that binds to BCMA and CD20; or
(g) a multispecific antibody molecule, e.g., a bispecific antibody molecule, that binds to CD20 and CD3, e.g., THG338,
(3) the antigen is CD22 and the inhibitor of the antigen is a CD22 inhibitor, optionally wherein the CD22 inhibitor is:
(h) a CD22 CAR-expressing cell therapy, e.g., a CD22 CAR-expressing cell therapy disclosed herein;
(i) a CAR-expressing cell therapy comprising a cell expressing a first CAR and a second CAR, wherein the first CAR is a BCMA CAR (e.g., a BCMA CAR disclosed herein) and the second CAR is a CD22 CAR (e.g., a CD22 CAR disclosed herein); or
(j) a CAR-expressing cell therapy comprising a cell expressing a multispecific CAR, e.g., a bispecific CAR, that binds to BCMA and CD22,
(4) the antigen is PD1 or PD-L1 and the inhibitor of the antigen is an anti-PD1 antibody molecule or an anti-PD-L1 antibody molecule, optionally wherein the inhibitor of the antigen is:
(k) PDR001, Nivolumab, Pembrolizumab, Pidilizumab, MEDI0680, REGN2810, TSR-042, PF-06801591, or AMP-224; or
(l) FAZ053, Atezolizumab, Avelumab, Durvalumab, or BMS-936559,
(5) the antigen is IDO or TDO and the inhibitor of the antigen is an inhibitor of IDO and/or TDO, optionally wherein the inhibitor of IDO and/or TDO is:
(m) INCB24360, indoximod, NLG919, epacadostat, NLG919, or F001287; or
(n) (4E)-4-[(3-chloro-4-fluoroanilino)-nitrosomethylidene]-1,2,5-oxadiazol-3-amine, 1-methyl-D-tryptophan, α-cyclohexyl-5H-Imidazo[5,1-a]isoindole-5-ethanol, or the D isomer of 1-methyl-tryptophan, or
(6) the antigen is TGF-beta and the inhibitor of the antigen is a TGF beta inhibitor.

83. A method of treating a subject having a disease associated with the expression of BCMA, comprising:
administering a BCMA CAR-expressing cell therapy to the subject,
acquiring a value of the level or activity of an antigen in the subject, e.g., in a sample from the subject (e.g., a biopsy sample, e.g., a bone marrow biopsy sample), at at least one time point after the subject began receiving the BCMA CAR-expressing cell therapy,
responsive to an increase in the value relative to a reference value, wherein the reference value is:
(i) the level or activity of the antigen in the subject, e.g., in a sample from the subject (e.g., a biopsy sample, e.g., a bone marrow biopsy sample), prior to the at least one time point (e.g., the level or activity of the antigen in the subject before the subject began receiving the BCMA CAR-expressing cell therapy, or the level or activity of the antigen in the subject after the subject began receiving the BCMA CAR-expressing cell therapy but prior to the at least one time point);
(ii) the level or activity of the antigen in a different subject having the disease associated with the expression of BCMA; or
(iii) an average level or activity of the antigen in a population of subjects having the disease associated with the expression of BCMA,
administering an inhibitor of the antigen to the subject, wherein:
(1) the antigen is CD19 and the inhibitor of the antigen is a CD19 inhibitor, optionally wherein the CD19 inhibitor is:
(a) a CD19 CAR-expressing cell therapy, e.g., a CD19 CAR-expressing cell therapy disclosed herein, e.g., CTL119 or CTL019;
(b) a CAR-expressing cell therapy comprising a cell expressing a first CAR and a second CAR, wherein the first CAR is a BCMA CAR (e.g., a BCMA CAR disclosed herein) and the second CAR is a CD19 CAR (e.g., a CD19 CAR disclosed herein); or
(c) a CAR-expressing cell therapy comprising a cell expressing a multispecific CAR, e.g., a bispecific CAR, that binds to BCMA and CD19,
(2) the antigen is CD20 and the inhibitor of the antigen is a CD20 inhibitor, optionally wherein the CD20 inhibitor is:
(d) a CD20 CAR-expressing cell therapy, e.g., a CD20 CAR-expressing cell therapy disclosed herein;
(e) a CAR-expressing cell therapy comprising a cell expressing a first CAR and a second CAR, wherein the first CAR is a BCMA CAR (e.g., a BCMA CAR disclosed herein) and the second CAR is a CD20 CAR (e.g., a CD20 CAR disclosed herein);
(f) a CAR-expressing cell therapy comprising a cell expressing a multispecific CAR, e.g., a bispecific CAR, that binds to BCMA and CD20; or
(g) a multispecific antibody molecule, e.g., a bispecific antibody molecule, that binds to CD20 and CD3, e.g., THG338,
(3) the antigen is CD22 and the inhibitor of the antigen is a CD22 inhibitor, optionally wherein the CD22 inhibitor is:
(h) a CD22 CAR-expressing cell therapy, e.g., a CD22 CAR-expressing cell therapy disclosed herein;
(i) a CAR-expressing cell therapy comprising a cell expressing a first CAR and a second CAR, wherein the first CAR is a BCMA CAR (e.g., a BCMA CAR disclosed herein) and the second CAR is a CD22 CAR (e.g., a CD22 CAR disclosed herein); or
(j) a CAR-expressing cell therapy comprising a cell expressing a multispecific CAR, e.g., a bispecific CAR, that binds to BCMA and CD22,
(4) the antigen is PD1 or PD-L1 and the inhibitor of the antigen is an anti-PD1 antibody molecule or an anti-PD-L1 antibody molecule, optionally wherein the inhibitor of the antigen is:
(k) PDR001, Nivolumab, Pembrolizumab, Pidilizumab, MEDI0680, REGN2810, TSR-042, PF-06801591, or AMP-224; or
(l) FAZ053, Atezolizumab, Avelumab, Durvalumab, or BMS-936559,
(5) the antigen is IDO or TDO and the inhibitor of the antigen is an inhibitor of IDO and/or TDO, optionally wherein the inhibitor of IDO and/or TDO is:
(m) INCB24360, indoximod, NLG919, epacadostat, NLG919, or F001287; or
(n) (4E)-4-[(3-chloro-4-fluoroanilino)-nitrosomethylidene]-1,2,5-oxadiazol-3-amine, methyl-D-tryptophan, α-cyclohexyl-5H-Imidazo[5,1-a]isoindole-5-ethanol, or the D isomer of 1-methyl-tryptophan, or
(6) the antigen is TGF-beta and the inhibitor of the antigen is a TGF beta inhibitor.

84. The method of any one of claims 80-83, wherein the value of the level or activity of the antigen comprises the expression level of the antigen in the subject, e.g., in a sample from the subject (e.g., a biopsy sample, e.g., a bone marrow biopsy sample), as measured by an assay described herein, e.g., immunohistochemistry.

85. The method of any one of claims 80-84, wherein the at least one time point is 5, 10, 15, 20, 25, 28, 30, 35, 40, 45, 50, 55, 60, 65, 70, 75, 80, or 90 days after the subject began receiving the BCMA CAR-expressing cell therapy.

86. The method of any one of claims 80-85, wherein the subject experiences a decrease in BCMA expression after the subject began receiving the BCMA CAR-expressing cell therapy.

87. The method of any one of claims 1-86, wherein the BCMA CAR-expressing cell therapy comprises a cell expressing a BCAM CAR, wherein:
(i) the BCMA CAR comprises one or more of (e.g., all three of) heavy chain complementary determining region 1 (HCDR1), HCDR2, and HCDR3 listed in Table 3 or 5 and/or one or more of (e.g., all three of) light chain complementary determining region 1 (LCDR1), LCDR2, and LCDR3 listed in Table 4 or 5, or a sequence with 95-99% identify thereof;
(ii) the BCMA CAR comprises a heavy chain variable region (VH) listed in Table 2 or 5 and/or a light chain variable region (VL) listed in Table 2 or 5, or a sequence with 95-99% identify thereof;
(iii) the BCMA CAR comprises a BCMA scFv domain amino acid sequence listed in Table 2 or 5 (e.g., SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, and SEQ ID NO: 149), or a sequence with 95-99% identify thereof;
(iv) the BCMA CAR comprises a full-length BCMA CAR amino acid sequence listed in Table 2 or 5 (e.g., residues 22-483 of SEQ ID NO: 109, residues 22-490 of SEQ ID NO: 99, residues 22-488 of SEQ ID NO: 100, residues 22-487 of SEQ ID NO: 101, residues 22-493 of SEQ ID NO: 102, residues 22-490 of SEQ ID NO: 103, residues 22-491 of SEQ ID NO: 104, residues 22-482 of SEQ ID NO: 105, residues 22-483 of SEQ ID NO: 106, residues 22-485 of SEQ ID NO: 107, residues 22-483 of SEQ ID NO: 108, residues 22-490 of SEQ ID NO: 110, residues 22-483 of SEQ ID NO: 111, residues 22-484 of SEQ ID NO: 112, residues 22-485 of SEQ ID NO: 113, residues 22-487 of SEQ ID NO: 213, residues 23-489 of SEQ ID NO: 214, residues 22-490 of SEQ ID NO: 215, residues 22-484 of SEQ ID NO: 216, residues 22-485 of SEQ ID NO: 217, residues 22-489 of SEQ ID NO: 218, residues 22-497 of SEQ ID NO: 219, residues 22-492 of SEQ ID NO: 220, residues 22-490 of SEQ ID NO: 221, residues 22-485 of SEQ ID NO: 222, residues 22-492 of SEQ ID NO: 223, residues 22-492 of SEQ ID NO: 224, residues 22-483 of SEQ ID NO: 225, residues 22-490 of SEQ ID NO: 226, residues 22-485 of SEQ ID NO: 227, residues 22-486 of SEQ ID NO: 228, residues 22-492 of SEQ ID NO: 229, residues 22-488 of SEQ ID NO: 230, residues 22-488 of SEQ ID NO: 231, residues 22-495 of SEQ ID NO: 232, residues 22-490 of SEQ ID NO: 233), or a sequence with 95-99% identify thereof; or
(v) the BCMA CAR is encoded by a nucleic acid sequence listed in Table 2 or 5 (e.g., SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170), or a sequence with 95-99% identify thereof.

88. The method of any one of claims 1-87, wherein the disease associated with the expression of BCMA is cancer, optionally wherein the cancer is a hematological cancer.

89. The method of any one of claims 1-88, wherein the disease associated with the expression of BCMA is an acute leukemia chosen from one or more of B-cell acute lymphoid leukemia (“BALL”), T-cell acute lymphoid leukemia (“TALL”), acute lymphoid leukemia (ALL); chronic myelogenous leukemia (CML), chronic lymphocytic leukemia (CLL); B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia; a prostate cancer (e.g., castrate-resistant or therapy-resistant prostate cancer, or metastatic prostate cancer), pancreatic cancer, lung cancer; or a plasma cell proliferative disorder (e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome)), or a combination thereof.

90. The method of any one of claims 1-89, wherein the disease associated with the expression of BCMA is ALL, CLL, DLBCL, or multiple myeloma.

91. The method of any one of claims 1-90, wherein the subject is a human patient.

92. A BCMA CAR-expressing cell therapy for use in a method of treatment of a subject having a disease associated with the expression of BCMA, the method comprising:
responsive to an increased value for one, two, three, four, five, or all of:
(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,
(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(iv) the level or activity of CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)), or
(vi) the proliferation of seeded cells from the subject during manufacturing of a BCMA CAR-expressing cell therapy,
as compared to a reference value, e.g., a non-responder reference value, performing:
manufacturing a BCMA CAR-expressing cell therapy using cells (e.g., T cells) from the subject and administering the BCMA CAR-expressing cell therapy to the subject; or
administering, e.g., initiating administering or continuing administering, a BCMA CAR-expressing cell therapy to the subject,
thereby treating the subject having the disease associated with the expression of BCMA.

93. A BCMA CAR-expressing cell therapy for use in a method of treatment of a subject having a disease associated with the expression of BCMA, the method comprising:
responsive to a decreased value for one, two, three, four, five, or all of:
(i) the level or activity of CD4+ immune effector cells (e.g., CD4+ T cells) relative to the level or activity of CD8+ immune effector cells (e.g., CD8+ T cells) in the subject, e.g., in a sample from the subject (e.g., an apheresis sample (e.g., a leukapheresis sample), in a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy,
(ii) the level or activity of CD8+ Tscm (stem cell memory T cells) in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(iii) the level or activity of HLADR-CD95+CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(iv) the level or activity of CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)),
(v) the level or activity of CCR7+CD45RO−CD27+CD8+ cells in the subject, e.g., in a sample from the subject (e.g., an apheresis sample (e.g., a leukapheresis sample) or a seed culture at the start of the manufacturing of a BCMA CAR-expressing cell therapy (e.g., a leukapheresis sample after monocytes are removed using elutriation)), or
(vi) the proliferation of seeded cells from the subject during manufacturing of a BCMA CAR-expressing cell therapy,
as compared to a reference value, e.g., a responder reference value, performing one, two, three, four, five, six, seven, or all of:
administering an altered dosing regimen of a BCMA CAR-expressing cell therapy (e.g., a dosing regimen with a higher dose and/or more frequent administration than a reference dosing regimen) to the subject;
administering a second therapy (e.g., a second therapy that is not a BCMA CAR-expressing cell therapy) to the subject;
administering a BCMA CAR-expressing cell therapy and a second therapy to the subject;
discontinuing administration of a BCMA CAR-expressing cell therapy and optionally administering a second therapy to the subject;
modifying a manufacturing process of a BCMA CAR-expressing cell therapy, e.g., enriching for CD4+ immune effector cells (e.g., CD4+ T cells) relative to CD8+ immune effector cells (CD8+ T cells) prior to introducing a nucleic acid encoding a BCMA CAR, and administering the BCMA CAR-expressing cell therapy generated by the modified manufacturing process to the subject;
modifying a manufacturing process of a BCMA CAR-expressing cell therapy, e.g., enriching for CD8+ Tscm (e.g., HLADR-CD95+CD27+CD8+ cells, CD45RO−CD27+CD8+ cells, or CCR7+CD45RO−CD27+CD8+ cells) prior to introducing a nucleic acid encoding a BCMA CAR, and administering the BCMA CAR-expressing cell therapy generated by the modified manufacturing process to the subject;
modifying a manufacturing process of the BCMA CAR-expressing cell therapy, e.g., increasing the proliferation of seeded cells from the subject during the manufacturing of the BCMA CAR-expressing cell therapy, and administering the BCMA CAR-expressing cell therapy generated by the modified manufacturing process to the subject; or
administering a pretreatment to the subject, wherein the pretreatment increases the ratio of the amount of CD4+ immune effector cells (e.g., CD4+ T cells) to the amount of CD8+ immune effector cells (e.g., CD8+ T cells) in the subject, e.g., in a sample from the subject (e.g., an apheresis sample (e.g., a leukapheresis sample), a seed culture at the start of the manufacturing of a BCMA CAR-expressing cell therapy (e.g., a leukapheresis sample after monocytes are removed using elutriation)), or in the subject's peripheral blood and/or bone marrow prior to the administration of the BCMA CAR-expressing cell therapy, e.g., the pretreatment increases the ratio to greater than or equal to 1.6 (e.g., between 1.6 and 5, e.g., between 1.6 and 3.5), manufacturing the BCMA CAR-expressing cell therapy using cells (e.g., T cells) from the subject, and administering the BCMA CAR-expressing cell therapy to the subject,
thereby treating the subject having the disease associated with the expression of BCMA.
